(19) United States
(12) Patent Application Publication
(10) Pub. No.: US 2019/0300967 A1
(43) Pub. Date: Oct. 3, 2019

(54) COMPOSITIONS AND METHODS FOR PREDICTING RESPONSE AND RESISTANCE TO CTLA4 BLOCKADE IN MELANOMA USING A GENE EXPRESSION SIGNATURE

(71) Applicant: DANA-FARBER CANCER INSTITUTE, INC., Boston, MA (US)

(72) Inventors: Pavan Bachireddy, Boston, MA (US); Sachet A. Shukla, Natick, MA (US); Catherine J. Wu, Brookline, MA (US)

(73) Assignee: DANA-FARBER CANCER INSTITUTE, INC., Boston, MA (US)

(21) Appl. No.: 16/340,969

(22) PCT Filed: Oct. 13, 2017
(86) PCT No.: PCT/US2017/056599

§ 371 Date: Apr. 10, 2019

Related U.S. Application Data
(60) Provisional application No. 62/407,591, filed on Oct. 13, 2016.
(60) Provisional application No. 62/565,411, filed on Sep. 29, 2017.

(51) Int. Cl. C12Q 1/6886; C07K 16/28; A61P 35/00

(52) U.S. Cl. CPC C12Q 1/6886; C07K 16/2818; A61P 35/00; A61K 2039/505; C12Q 2600/158; C12Q 2600/154; C07K 2317/76; C12Q 2600/106

(57) ABSTRACT

The present invention relates to compositions and methods for predicting response and resistance to CTA4 blockade in melanoma.

### RELATED APPLICATIONS

This application claims the benefit of priority under 35 U.S.C. § 119(e) to U.S. Provisional Application No. 62/407,591, filed Oct. 13, 2016, and to U.S. Provisional Application No. 62/565,411, filed Sep. 29, 2017, each of which is incorporated herein by reference in its entirety.

### GOVERNMENT LICENSE RIGHTS

This invention was made with government support under grant number 1R01CA182461-01 awarded by the National Cancer Institute, under grant number 1R01CA184922-02 awarded by the National Cancer Institute, under grant number R50RCA211482A awarded by the National Cancer Institute, and under grant number 1R01CA155010-05 awarded by the National Cancer Institute. The government has certain rights in the invention.

### BACKGROUND OF THE INVENTION

Cytotoxic T-lymphocyte-associated protein 4 (CTLA4) blockade can induce durable clinical remissions in a minority of patients with metastatic melanoma. However, prior to the invention described herein, molecular signatures precisely predicting response and resistance to CTLA4 blockade were unknown. As such, there is a pressing need to identify more effective methods for predicting response or resistance to CTLA4 blockade.

### SUMMARY OF THE INVENTION

The invention is based, at least in part, upon the identification of a gene expression signature that discriminates clinical outcomes of CTLA4 blockade. Specifically, described herein is a specific cluster of cancer-testis antigens and microRNA-211 that are predictive of resistance and response, respectively, to ipilimumab in melanoma. In some aspects, the invention relates to methods, arrays, and kits for diagnosing, monitoring, and treating melanoma.

As described in detail below, in one aspect, the invention is a gene expression signature that predicts clinical response and resistance to CTLA4 blockade, e.g., ipilimumab, in patients with metastatic melanoma.

In one aspect, increased expression of at least one of the following genes significantly correlates with resistance to ipilimumab: MAGEA2, CSAG4, MAGEA2B, AC093787 (RP11-215P9), MAGEA12, CSAG1, GABRA3, CSAG3, makorin ring finger protein 9 (MKRN9P), keratin 8 pseudogene 8 (KRT8P8), MAGEA6, EYA1, CSAG2, RP11-379D21.3, MAGE family member C1 (MAGEC1), RP1-273G13.1, MAGEA3, miR-218-1, pregnancy specific beta-1-glycoprotein 11 (PSG11), X-inactive specific transcript (XIST), RP11-360D2.1, pregnancy specific beta-1-glycoprotein 10 pseudogene (PSG10P), miR-1262, tachykinin 3 (TAC3), PSG8, heat shock protein family B (small) member 3 (HSPB3), gap junction protein beta-6 (GJB6), GABRQ, MAGEA1, MAGEA11, MAGEA9B, and PSG6.

A cluster of CT antigen genes on the Xq28 cytoband (i.e., MAGEA2, CSAG4, MAGEA2B, MAGEA12, CSAG1, CSAG3, MAGEA6, CSAG2, MAGEA3) correlate with resistance to ipilimumab. Additionally, miR-211 and transient receptor potential cation channel subfamily M member 1 (TRPM1) (which subsumes miR-211) correlate with response to ipilimumab.

Also provided is a gene expression signature that predicts clinical response and resistance to a combination of an agonist of an HMGB1 pathway, HMGB1 receptor (henceforth “HMGB1 agonist”)(e.g., toll-like receptor (TLR) agonists); or agonist of autophagy (e.g., metformin, temozolomide, trifluoperazine, divalproex sodium, vorinostat, rapamycin, everolimus, MG-132, doxorubicin, ABT-737, BCL2 inhibitors/antagonists, gemcitabine, torin 1, resveratrol, etc); or an agonist of miR-211, miR-185 and/or miR-513A2; or Xq28-CGA antagonist and CTLA4 blockade, e.g., ipilimumab or tremelimumab, in patients with melanoma, e.g., metastatic melanoma. Specifically, increased expression of at least one of the following genes significantly correlates with benefit to treatment with a TLR agonist (or autophagy agonist or Xq28-CGA antagonist) and ipilimumab: MAGEA2, MAGE2AB, MAGEA3, MAGEA6, MAGEA12, CSAG1, CSAG2, and CSAG3. For example, the Xq28-CGA inhibitor comprises an antibody, an aptamer, or a small molecule. Additionally, decreased expression of at least one of these genes significantly correlates with benefit to treatment with an agonist of miR-211, miR-185 and/or miR-513A2. For example, the miR agonist comprises a miR mimetic (natural or synthetic) or an aptamer.

Accordingly, provided is a method of determining whether inhibition of cytotoxic T-lymphocyte-associated protein 4 (CTLA4) in a subject, e.g., a human subject, with melanoma will result in clinical benefit (e.g., inhibition of melanoma cancer cells) in the subject, comprising: obtaining a test sample from a subject having or at risk of developing melanoma; determining the expression level of at least one melanoma-associated gene in the test sample; comparing the expression level of the melanoma-associated gene in the test sample with the expression level of the melanoma-associated gene in a reference sample; and determining whether CTLA4 blockade will inhibit melanoma in the subject if the expression level of the melanoma-associated gene in the test sample is differentially expressed as compared to the level of the melanoma-associated gene in the reference sample.

Also provided is a method of treating cancer comprising administering an effective amount of a CTLA4 inhibitor and an effective amount of an HMGB1 agonist or autophagy agonist or Xq28-CGA antagonist. For example, the CTLA4 inhibitor comprises ipilumamab. In another example, the HMGB1 agonist comprises high mobility group box 1 (HMGB1), TLR agonists like unmethylated CpG DNA (e.g., CpG-oligodeoxynucleotides or CpG-ODN), Hiltonol (poly-ICLC), Bacillus Calmette-Guérin (BCG), monophosphoryl lipid A (MPL), imiquimod, etc. In other example, the agonist of autophagy comprises inducers of autophagy, e.g., metformin, temozolomide, trifluoperazine, divalproex sodium, vorinostat, mTOR inhibitors (e.g., rapamycin, everolimus), MG-132, doxorubicin, ABT-737, BCL2 inhibitors/antagonists, gemcitabine, torin 1, resveratrol, etc. In other example, the agonist of miR-211, miR-185 and/or miR-513A2 comprises a miR mimetic (synthetic or natural) or an aptamer.

Also provided are methods of determining whether administration of a CTLA4 inhibitor and an HMGB1 agonist to a subject with melanoma will result in clinical benefit in the subject comprising obtaining a test sample from a subject having or at risk of developing melanoma; determining the expression level of at least one melanoma-associated gene in the test sample; comparing the expression level of the melanoma-associated gene in the test sample with the expression level of the melanoma-associated gene in a reference sample; and determining whether administration of a CTLA4 inhibitor and an HMGB1 agonist will inhibit melanoma in the subject if the expression level of the melanoma-associated gene in the test sample is differentially expressed as compared to the level of the melanoma-associated gene in the reference sample.

For example, the test sample is obtained from the melanoma, wherein the melanoma-associated gene comprises a cancer germline antigen (CGA) gene; and determining that administration of the CTLA4 inhibitor and the HMGB1 agonist in a subject with melanoma will result in clinical benefit in the subject if the expression level of the CGA gene in the test sample is higher than the level of the CGA gene in the reference sample.

In one aspect, the CGA gene comprises MAGEA2, MAGEA3, MAGEA6, MAGEA12, CSAG1, CSAG2, or CSAG3.

Alternatively, the expression level of the melanoma-associated gene in the test sample is compared with a threshold expression level of the melanoma-associated gene (e.g., a “cut-off level”). The method involves determining whether CTLA4 blockade will inhibit melanoma in the subject if the expression level of the melanoma-associated gene in the test sample is differentially expressed as compared to the threshold expression level of the melanoma-associated gene.

In another case, the expression level of the melanoma-associated gene in the test sample is compared with an expression level of a housekeeping gene within the test sample. The method involves determining whether CTLA4 blockade will inhibit melanoma in the subject if the expression level of the melanoma-associated gene in the test sample is differentially expressed as compared to the expression level of the housekeeping gene. For example, clinical benefit in the subject comprises complete or partial response or stable disease with overall survival of greater than one year as defined by response evaluation criteria in solid tumors (RECIST). In some cases, clinical benefit is associated with an inhibition of melanoma cells. By contrast, the absence of clinical benefit (i.e., no clinical benefit) in the subject comprises progressive disease or stable disease with overall survival of less than one year as defined by RECIST. Alternatively or in addition to using RECIST, clinical benefit in the subject is evaluated using immune-related response criteria (irRC). For example, clinical benefit comprises long-term survival in spite of disease progression or response defined by irRC criteria

The expression level of the melanoma-associated gene in the test sample is differentially expressed as compared to the level of the melanoma-associated gene in the reference sample, the threshold expression level, or the expression level of a housekeeping gene. For example, the expression level of the melanoma-associated gene in the test sample is upregulated (i.e., increased) by at least 2 fold, at least 3 fold, at least 4 fold, at least 5 fold, at least 6 fold, at least 7 fold, at least 8 fold, at least 9 fold, at least 10 fold, at least 15 fold, at least 20 fold, at least 25 fold, at least 30 fold, at least 35 fold, at least 40 fold, at least 45 fold, at least 50 fold, at least 60 fold, at least 70 fold, at least 80 fold, at least 90 fold, at least 100 fold, at least 125 fold, at least 150 fold, at least 175 fold, at least 200 fold, at least 250 fold, at least 300 fold, at least 350 fold, at least 400 fold, at least 500 fold, at least 600 fold, at least 700 fold or at least 800 fold as compared to the level of the melanoma-associated gene in the reference sample, the threshold expression level, or the expression level of a housekeeping gene.

Alternatively, the expression level of the melanoma-associated gene in the test sample is downregulated (i.e., decreased) by at least 2 fold, at least 3 fold, at least 4 fold, at least 5 fold, at least 6 fold, at least 7 fold, at least 8 fold, at least 9 fold, at least 10 fold, at least 15 fold, at least 20 fold, at least 25 fold, at least 30 fold, at least 35 fold, at least 40 fold, at least 45 fold, at least 50 fold, at least 60 fold, at least 70 fold, at least 80 fold, at least 90 fold, at least 100 fold, at least 125 fold, at least 150 fold, at least 175 fold, at least 200 fold, at least 250 fold, at least 300 fold, at least 350 fold, at least 400 fold, at least 500 fold, at least 600 fold, at least 700 fold or at least 800 fold as compared to the level of the melanoma-associated gene in the reference sample, the threshold expression level, or the expression level of a housekeeping gene.

In one aspect, the test sample is obtained from the melanoma tissue, from the tumor microenvironment, or from tumor-infiltrating immune cells. For example, the test sample is obtained from the melanoma and the melanoma-associated gene comprises a gene on chromosome Xq28. For example, the melanoma-associated gene comprises a cancer germline antigen (CGA) gene (i.e., a cancer-testis (CT) antigen gene); and the method involves determining that inhibition of CTLA4 in a subject with melanoma will not result in clinical benefit in the subject if the expression level of the CGA gene in the test sample is higher than the level of the CGA gene in the reference sample. Exemplary CGA genes include melanoma-associated antigen 2 (MAGEA2), MAGEA3, MAGEA6, MAGEA12, chondrosarcoma associated gene 1 (CSAG1), CSAG2, CSAG3, and CSAG4.

Optionally, the melanoma-associated gene is hypomethylated, e.g., there is a decrease in the epigenetic methylation of cytosine residues in CpG dinucleotides deoxyribonucleic acid (DNA) in the promoter and/or a change in epigenetic methylation of cytosine residues in CpG dinucleotides in the gene body. For example, a CGA gene is hypomethylated in the promoter. For example, local hypomethylation of the Xq28 MAGE genes described herein, e.g., MAGEA2, MAGEA3, MAGEA6, or MAGEA12, is identified. Alternatively, or in addition, global hypomethylation of the genes in the test sample is identified. As described herein, hypomethylation of genes is an indication that inhibition of CTLA4 in a subject with melanoma will not result in clinical benefit in the subject.

Optionally, the melanoma-associated gene is hypermethylated, e.g., there is an increase in the epigenetic methylation of cytosine residues in CpG dinucleotides deoxyribonucleic acid (DNA) in the promoter and/or a change in epigenetic methylation of cytosine residues in CpG dinucleotides in the gene body. For example, a CGA gene is hypermethylated in the promoter. For example, local hypermethylation of the Xq28 MAGE genes described herein is identified. Alternatively, or in addition, global hypermethylation of the genes in the test sample is identified. As described herein, hypermethylation of genes is an indication that inhibition of CTLA4 in a subject with melanoma will result in clinical benefit in the subject.

Alternatively, the test sample is obtained from the melanoma and the melanoma-associated gene comprises a pregnancy-specific glycoprotein (PSG) gene, a γ-aminobutyric acid (GABA) A receptor gene, an epithelial-to-mesenchymal transition gene, an embryonic development/differentiation gene, an angiogenesis gene, or an extracellular matrix (ECM) gene; and the method involves determining that inhibition of CTLA4 in a subject with melanoma will not result in clinical benefit in the subject if the expression level of the PSG gene, GABA A receptor gene, epithelial-to-mesenchymal transition gene, embryonic development/differentiation gene, angiogenesis gene, or extracellular matrix gene in the test sample is higher than the level of the respective gene in the reference sample.

Exemplary PSG genes include PSG1, PSG2, PSG4, PSG5, PSG6, PSG7, PSG8, PSG9, and PSG11. In some cases, the PSG gene is hypomethylated. Suitable GABA A receptor genes include gamma-aminobutyric acid type A receptor alpha 3 subunit (GABRA3), gamma-aminobutyric acid type A receptor beta 1 subunit (GABRB1), GABRB2, gamma-aminobutyric acid type A receptor gamma 2 subunit (GABRG2), gamma-aminobutyric acid type A receptor theta subunit (GABRQ), gamma-aminobutyric acid type A receptor rho 1 subunit (GABRR1). In one aspect, the epithelial-to-mesenchymal transition gene comprises claudin 1 (CLDN1), CLDN2, eyes absent homolog 1 (EYA1), snail family zinc finger 1 (SNAI1), transforming growth factor beta 2 (TGFB2), or wingless-type MMTV integration site family member 3 (WNT3). Exemplary embryonic development/differentiation genes include homeobox D13 (HOXD13), HOXD11, HOXA2, HOXA5, and HOXD10. In some cases, the angiogenesis gene comprises angiopoietin 1 (ANGPT1), angiopoietin-2 (ANG2), or platelet derived growth factor subunit A (PDGFA). Suitable ECM genes include protocadherin beta 2 (PCDHB2), PCDHB3, PCDHB6, PCDHB10, protocadherin gamma subfamily A3, (PCDHGA3), PCDHGB1, PCDHGB2, elastin microfibril interfacer 1 (EMILIN1), and tenascin N (TNN).

In other cases, the test sample is obtained from the melanoma, and the melanoma-associated gene comprises micro ribonucleic acid-211 (miR-211), miR-513A2, or miR-185. It is determined that inhibition of CTLA4 in a subject with melanoma will result in clinical benefit in the subject if the expression level of miR-211, miR-513A2, or miR-185 in the test sample is higher than the level of miR-211, miR-513A2, or miR-185, respectively, in the reference sample.

In other cases, the test sample is obtained from the melanoma, and the melanoma-associated gene comprises melastatin-1 (TRPM1). It is determined that inhibition of CTLA4 in a subject with melanoma will result in clinical benefit in the subject if the expression level of TRPM1 in the test sample is higher than the level of TRPM1 in the reference sample.

In one aspect, the test sample is obtained from the melanoma and the melanoma-associated gene comprises miR-211, cluster of differentiation 5 molecule like (CD5L), interleukin 12 receptor subunit beta 2 (IL12RB2), fas apoptotic inhibitory molecule 3 (FAIM3), and/or pre T-Cell antigen receptor alpha (PTCRA). It is determined that inhibition of CTLA4 in a subject with melanoma will result in clinical benefit in the subject if the expression level of miR-211, CD5L, IL12RB2, FAIM3, and PTCRA in the test sample is higher than the level of the corresponding gene in the reference sample.

In another aspect, the test sample is obtained from the melanoma, and the melanoma-associated gene comprises miR-211, MAGEA2, MAGEA3, MAGEA6, MAGEA12, CSAG1, CSAG2, CSAG3, or CSAG4. It is determined that inhibition of CTLA4 in a subject with melanoma will not result in clinical benefit in the subject if the expression level of miR-211 in the test sample is lower than the level of miR-211 in the reference sample and if the expression level of MAGEA2, MAGEA3, MAGEA6, MAGEA12, CSAG1, CSAG2, CSAG3, and CSAG4 in the test sample is higher than the level of the corresponding gene in the reference sample.

Alternatively, the test sample is obtained from a melanoma or the infiltrating immune cells, wherein the melanoma-associated gene comprises a T cell infiltration-associated gene, a receptor signaling gene, an activation gene, a cytotoxicity gene, a humoral immunity gene, and/or an immune inhibitory receptor gene. It is determined whether inhibition of CTLA4 in a subject with melanoma will result in clinical benefit in the subject if the expression level of the T cell infiltration-associated gene, receptor signaling gene, activation gene, or cytotoxicity gene in the test sample is higher than the level of the corresponding gene in the reference sample.

Suitable T cell infiltration-associated genes include cluster of differentiation 2 (CD2), CD6, and C-X-C motif chemokine ligand 13 (CXCL13). Exemplary receptor signaling genes include CD3D, CD3E, CD3G, lymphocyte-specific protein tyrosine kinase (LCK), T cell receptor alpha gene, T cell receptor beta gene, and PTCRA. Suitable activation genes include CD28, inducible t-cell co-stimulator (ICOS), eomesodermin (EOMES), interleukin-2 receptor subunit beta (IL2RB), Fas ligand (FASLG), and signaling lymphocytic activation molecule family member 6 (SLAMF6). In one aspect, cytotoxicity genes include granulysin (GNLY), granzyme A (GZMA), GZMB, GZMH, GZMK, and perforin 1 (PRF1). Suitable humoral immunity genes include CD19, CD72, Fc receptor-like protein 1/3 (FCRL1/3), and membrane spanning 4-domains A1 (MS4A1).

In some cases, immune inhibitory receptors include a receptor specific to or preferentially expressed by T cells such as CTLA4 and lymphocyte-activation gene-3 (LAG3). Alternatively, the immune inhibitory receptor comprises a receptor specific to or preferentially expressed by B cells such as CTLA4, FCRL1, and FCRL3. In other cases, the immune inhibitory receptor comprises a receptor specific to or preferentially expressed by macrophages such as CD5L. In other aspects, the immune inhibitory receptor comprises a receptor specific to or preferentially expressed by eosinophils/mast cells such as sialic acid-binding Ig-like lectin 8 (SIGLEC8). Alternatively, the immune inhibitory receptor comprises fas apoptotic inhibitory molecule 3 (FAIM3/TOSO).

In one aspect, the test sample is obtained from the melanoma and the melanoma-associated gene comprises CD2, CD6, CXCL13, CD3D, CD3E, CD3G, LCK, T cell receptor alpha gene, T cell receptor beta gene, CD28, ICOS, EOMES, IL2RB, FASLG, SLAMF6, GNLY, GZMA, GZMB, GZMH, GZMK, PRF1, PTCRA, CD19, CD72, FCRL1/3, MS4A1, CTLA4, LAG3, FCRL1, FCRL3, CD5L, SIGLEC8, and/or FAIM3/TOSO (or any combination thereof). It is determined that inhibition of CTLA4 in a subject with melanoma will result in clinical benefit in the subject if the expression level of CD2, CD6, CXCL13, CD3D, CD3E, CD3G, LCK, T cell receptor alpha gene, T cell receptor beta gene, CD28, ICOS, EOMES, IL2RB, FASLG, SLAMF6, GNLY, GZMA, GZMB, GZMH, GZMK, PRF1, PTCRA, CD19, CD72, FCRL1/3, MS4A1, CTLA4, LAG3, FCRL1, FCRL3, CD5L, SIGLEC8, and/or FAIM3/TOSO (or any combination thereof) in the test sample is higher than the level of the corresponding gene in the reference sample.

Alternatively, the test sample is obtained from the melanoma and the melanoma-associated gene comprises miR-211, along with one or more of CD2, CD6, CXCL13, CD3D, CD3E, CD3G, LCK, T cell receptor alpha gene, T cell receptor beta gene, CD28, ICOS, EOMES, IL2RB, FASLG, SLAMF6, GNLY, GZMA, GZMB, GZMH, GZMK, PRF1, PTCRA, CD19, CD72, FCRL1/3, MS4A1, CTLA4, LAG3, FCRL1, FCRL3, CD5L, SIGLEC8, and/or FAIM3/TOSO (or any combination thereof). It is determined that inhibition of CTLA4 in a subject with melanoma will result in clinical benefit in the subject if the expression level of miR-211, along with one or more of CD2, CD6, CXCL13, CD3D, CD3E, CD3G, LCK, T cell receptor alpha gene, T cell receptor beta gene, CD28, ICOS, EOMES, IL2RB, FASLG, SLAMF6, GNLY, GZMA, GZMB, GZMH, GZMK, PRF1, PTCRA, CD19, CD72, FCRL1/3, MS4A1, CTLA4, LAG3, FCRL1, FCRL3, CD5L, SIGLEC8, and/or FAIM3/TOSO (or any combination thereof) in the test sample is higher than the level of the corresponding gene in the reference sample.

Suitable samples include those with deoxyribonucleic acid (DNA) or ribonucleic acid (RNA) therein. For example, the sample is a tumor sample. In anther aspect, the sample is a tumor microenvironment sample. Optionally, the sample is a plasma sample or a blood sample. In some cases, the sample comprises one or more circulating tumor cells.

In some cases, the reference sample is obtained from healthy normal tissue, melanoma that received a clinical benefit from CTLA4 inhibition, or melanoma that did not receive a clinical benefit from CTLA4 inhibition.

Optionally, the expression level of the melanoma-associated gene is detected via an Affymetrix Gene Array hybridization, next-generation sequencing, ribonucleic acid sequencing (RNA-seq), a real time reverse transcriptase polymerase chain reaction (real time RT-PCR) assay, immunohistochemistry (IHC), immunofluorescence, or methylation-specific PCR.

In one aspect, the expression level of the melanoma-associated gene is detected via RNA-seq and the reference sample is obtained from healthy normal tissue from the same individual as the test sample or one or more healthy normal tissues from different individuals.

In other cases, the expression level of the melanoma-associated gene is detected via RT-PCR and the reference sample is obtained from the same tissue as the test sample. In this case, levels of a housekeeping gene are determined in the reference sample. Suitable housekeeping genes include glyceraldehyde 3-phosphate dehydrogenase (GAPDH), hypoxanthine phosphoribosyltransferase 1 (HPRT1), and serine/threonine protein kinase (PSK1). The method involves determining whether CTLA4 blockade will inhibit melanoma in the subject if the expression level of the melanoma-associated gene in the test sample is differentially expressed as compared to the expression level of the housekeeping gene.

The methods described herein optionally further comprise treating the subject with a chemotherapeutic agent, radiation therapy, cryotherapy, or hormone therapy. Exemplary chemotherapeutic agents include dacarbazine, temozolomide, nab-paclitaxel, paclitaxel, cisplatin, or carboplatin.

In some cases, the methods described herein further comprise administering an inhibitor of the melanoma-associated gene with a higher level of expression compared to the level of the melanoma-associated gene in the reference sample, thereby treating the melanoma. Suitable inhibitors include a small molecule inhibitor, RNA interference (RNAi), an antibody, an antibody fragment, an antibody drug conjugate, an aptamer, a chimeric antigen receptor (CAR), a T cell receptor, or any combination thereof.

In some cases, the antibody or antibody fragment is partially humanized, fully humanized, or chimeric. For example, the antibody fragment is a nanobody, an Fab, an Fab′, an (Fab′)2, an Fv, a single-chain variable fragment (ScFv), a diabody, a triabody, a tetrabody, a Bis-scFv, a minibody, an Fab2, an Fab3 fragment, or any combination thereof.

Alternatively, the methods described herein further comprise administering an agonist of the melanoma-associated gene with a higher level of expression compared to the level of the melanoma-associated gene in the reference sample, thereby treating the melanoma.

Optionally, the methods include administering to the subject a CTLA4 inhibitor, thereby treating the melanoma. For example, the CTLA4 inhibitor is an anti-CTLA4 antibody, e.g., ipilimumab or tremelimumab.

Also provided are compositions for predicting no clinical benefit in response to CTLA4 therapy comprising a melanoma-associated gene. For example, the melanoma-associated gene comprises MAGEA2, MAGEA3, MAGEA6, MAGEA12, CSAG1, CSAG2, CSAG3, or CSAG4 synthesized complementary deoxyribonucleic acid (cDNA).

In some cases, the composition further comprises PSG1, PSG2, PSG4, PSG5, PSG6, GABRA3, GABRB1, GABRB2, GABRG2, GABRQ, GABRR1, CLDN1, CLDN2, EYA1, SNAI1, TGFB2, WNT3, HOXD13, HOXD11, HOXA2, HOXA5, HOXD10, ANGPT1, ANG2, PDGFA, PCDHB2, PCDHB3, PCDHB6, PCDHB10, PCDHGA3, PCDHGB1, PCDHGB2, EMILIN1, and/or TNN synthesized cDNA.

Also provided are compositions for predicting clinical benefit in response to CTLA4 therapy comprising miR-211 and a melanoma-associated gene selected from the group consisting of CD5L, IL12RB2, FAIM3, PTCRA, CD2, CD6, CXCL13, CD3D, CD3E, CD3G, LCK, T cell receptor alpha gene, T cell receptor beta gene, GNLY, GZMA, GZMB, GZMH, GZMK, PRF1, CD19, CD72, FCRL1/3, MS4A1, CTLA4, LAG3, FCRL1, FCRL3, SIGLEC8, and FAIM3/TOSO synthesized cDNA.

In one aspect, the melanoma-associated gene is immobilized on a solid support. Optionally, the melanoma-associated gene is linked to a detectable label. Exemplary detectable labels include a fluorescent label, a luminescent label, a chemiluminescent label, a radiolabel, a SYBR Green label, and a Cy3-label.

Preferably, the compositions comprising melanoma-associated genes include synthetic or non-naturally occurring melanoma-associated genes.

Provided is a method of treating cancer in a subject in need thereof, comprising: administering a therapeutically effective amount of one or more CTLA4 inhibitor agents to the subject, wherein the subject is identified as (a) not having aberrant expression of at least one resistant cancer-associated gene or miRNA, or (b) having aberrant expression of at least one beneficial cancer-associated gene or miRNA.

Also provided is a method of treating cancer in a subject in need thereof, comprising: (a) analyzing a biological sample from the subject for: (i) aberrant expression of at least one resistant cancer-associated gene or miRNA, wherein the aberrant expression of the at least one resistant cancer-associated gene or miRNA is not present in the biological sample, or (ii) aberrant expression of at least one beneficial cancer-associated gene or miRNA, wherein the aberrant expression of the at least one beneficial cancer-associated gene or miRNA is present in the biological sample; (b) identifying the subject as a candidate for receiving one or more CTLA4 inhibitor agents; and (c) administering a therapeutically effective amount of the one or more CTLA4 inhibitor agents to the subject.

The invention provides a method of identifying a subject with cancer as a candidate for receiving one or more CTLA4 inhibitor agents, comprising: (a) analyzing a biological sample from the subject for: (i) aberrant expression of at least one resistant cancer-associated gene or miRNA, wherein the aberrant expression of the at least one resistant cancer-associated gene or miRNA is not present in the biological sample, or (ii) aberrant expression of at least one beneficial cancer-associated gene or miRNA, wherein the aberrant expression of the at least one beneficial cancer-associated gene or miRNA is present in the biological sample; and (b) identifying the subject as a candidate for receiving one or more ctla4 inhibitor agents.

Also provided is a method to predict a response of a subject with cancer to a CTLA4 therapy, the method comprising: (a) assaying for (i) aberrant expression of at least one resistant cancer-associated gene or miRNA in a biological sample from the subject, wherein the aberrant expression of the at least one resistant cancer-associated gene or miRNA is not present in the biological sample, or (ii) aberrant expression of at least one beneficial cancer-associated gene or miRNA in a biological sample from the subject, wherein the aberrant expression of the at least one beneficial cancer-associated gene or miRNA is present in the biological sample; and (b) predicting a response of the subject with cancer to a CTLA4 therapy to be positive based on the assaying.

Described herein is a method of treating cancer comprising administering an effective amount of a CTLA4 inhibitor and an effective amount of an agonist (or inducer) of autophagy. For example, the CTLA4 inhibitor comprises ipilimumab or tremelimumab. In some cases, the autophagy agonist comprises metformin, temozolomide, trifluoperazine, divalproex sodium, vorinostat, rapamycin, everolimus, MG-132, doxorubicin, ABT-737, BCL2 inhibitors/antagonists, gemcitabine, torin 1, or resveratrol, etc.

Also provided herein are methods of determining whether administration of a CTLA4 inhibitor and an autophagy agonist to a subject with melanoma will result in clinical benefit in the subject comprising obtaining a test sample from a subject having or at risk of developing melanoma; determining the expression level of at least one melanoma-associated gene in the test sample; comparing the expression level of the melanoma-associated gene in the test sample with the expression level of the melanoma-associated gene in a reference sample; and determining whether administration of a CTLA4 inhibitor and an autophagy agonist will inhibit melanoma in the subject if the expression level of the melanoma-associated gene in the test sample is differentially expressed as compared to the level of the melanoma-associated gene in the reference sample. For example, the autophagy agonist comprises metformin, temozolomide, trifluoperazine, divalproex sodium, vorinostat, rapamycin, everolimus, MG-132, doxorubicin, ABT-737, BCL2 inhibitors/antagonists, gemcitabine, torin 1, or resveratrol, etc.

In some cases, the test sample is obtained from the melanoma, wherein the melanoma-associated gene comprises a cancer germline antigen (CGA) gene; and the method comprises determining that administration of the CTLA4 inhibitor and the autophagy agonist in a subject with melanoma will result in clinical benefit in the subject if the expression level of the CGA gene in the test sample is higher than the level of the CGA gene in the reference sample. For example, the CGA gene comprises MAGEA2, MAGEA3, MAGEA6, MAGEA12, CSAG1, CSAG2, or CSAG3.

Also provided are methods of treating cancer comprising administering an effective amount of a CTLA4 inhibitor and an effective amount of a agonist (or inducer) of miR-211, miR-185 and/or miR-513A2. For example, the CTLA4 inhibitor comprises ipilimumab or tremelimumab. In some cases, the agonist of miR-211, miR-185 and/or miR-513A2 comprises a miR mimetic (natural or synthetic) or aptamer.

Also provided are methods of determining whether administration of a CTLA4 inhibitor and a miR-211, miR-185, or miR-513A2) agonist to a subject with melanoma will result in clinical benefit in the subject comprising: obtaining a test sample from a subject having or at risk of developing melanoma; determining the expression level of at least one melanoma-associated gene in the test sample; comparing the expression level of the melanoma-associated gene in the test sample with the expression level of the melanoma-associated gene in a reference sample; and determining whether administration of a CTLA4 inhibitor and an miR-211, miR-185 and/or miR-513A2 agonist will inhibit melanoma in the subject if the expression level of the melanoma-associated gene in the test sample is differentially expressed as compared to the level of the melanoma-associated gene in the reference sample. For example, the miR-211, miR-185 and/or miR-513A2 agonist comprises a miR mimetic (natural or synthetic) or aptamer.

In some cases, the test sample is obtained from the melanoma, wherein the melanoma-associated gene comprises a micro RNA gene; and determining that administration of the CTLA4 inhibitor and the miR-211, miR-185, and/or miR-513A2 agonist in a subject with melanoma will result in clinical benefit in the subject if the expression level of the miR-211, miR-513A2, or miR-185 in the test sample is higher than the level of the miR-211, miR-185 and/or miR-513A2 in the reference sample.

In other cases, the test sample is obtained from the melanoma, wherein the melanoma-associated gene comprises a melastatin-1 (TRPM1) gene; and determining that administration of the CTLA4 inhibitor and the miR-211, miR-185, and/or miR-513A2 agonist in a subject with melanoma will result in clinical benefit in the subject if the expression level of the TRPM1 gene in the test sample is higher than the level of the TRPM1 gene in the reference sample.

Provided herein are kits comprising reagents for assaying a biological sample from a subject with cancer for: (a) aberrant expression of at least one resistant cancer-associated gene or miRNA, or (b) aberrant expression of at least one beneficial cancer-associated gene or miRNA.

In one aspect, the aberrant expression of the at least one resistant cancer-associated gene or miRNA comprises overexpression of the at least one resistant cancer-associated gene or miRNA.

In another aspect, the aberrant expression of the at least one resistant cancer-associated gene is characterized by expression from a hypomethylated form of the at least one resistant cancer-associated gene.

In some cases, the aberrant expression of at least one beneficial cancer-associated gene or miRNA comprises overexpression of the at least one beneficial cancer-associated gene or miRNA.

### Definitions

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term “about.”

The phrase “aberrant expression” is used to refer to an expression level that deviates from (i.e., an increased or decreased expression level) the normal reference expression level of the gene.

The term “antineoplastic agent” is used herein to refer to agents that have the functional property of inhibiting a development or progression of a neoplasm in a human, e.g., a melanoma. Inhibition of metastasis is frequently a property of antineoplastic agents.

By “agent” is meant any small compound, antibody, nucleic acid molecule, or polypeptide, or fragments thereof.

By “alteration” is meant a change (increase or decrease) in the expression levels or activity of a gene or polypeptide as detected by standard art-known methods such as those described herein. As used herein, an alteration includes at least a 1% change in expression levels, e.g., at least a 2%, 3%, 4%, 5%, 6%, 7%, 8%, 9%, 10%, 20%, 30%, 40%, 50%, 60%, 70%, 80%, 90%, or 100% change in expression levels. For example, an alteration includes at least a 5%-10% change in expression levels, preferably a 25% change, more preferably a 40% change, and most preferably a 50% or greater change in expression levels.

By “ameliorate” is meant decrease, suppress, attenuate, diminish, arrest, or stabilize the development or progression of a disease.

The term “antibody” (Ab) as used herein includes monoclonal antibodies, polyclonal antibodies, multispecific antibodies (e.g., bispecific antibodies), and antibody fragments, so long as they exhibit the desired biological activity. The term “immunoglobulin” (Ig) is used interchangeably with “antibody” herein.

An “isolated antibody” is one that has been separated and/or recovered from a component of its natural environment. Contaminant components of its natural environment are materials that would interfere with diagnostic or therapeutic uses for the antibody, and may include enzymes, hormones, and other proteinaceous or nonproteinaceous solutes. In preferred embodiments, the antibody is purified: (1) to greater than 95% by weight of antibody as determined by the Lowry method, and most preferably more than 99% by weight; (2) to a degree sufficient to obtain at least 15 residues of N-terminal or internal amino acid sequence by use of a spinning cup sequenator; or (3) to homogeneity by SDS-PAGE under reducing or non-reducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

The basic four-chain antibody unit is a heterotetrameric glycoprotein composed of two identical light (L) chains and two identical heavy (H) chains. An IgM antibody consists of 5 of the basic heterotetramer unit along with an additional polypeptide called J chain, and therefore contain 10 antigen binding sites, while secreted IgA antibodies can polymerize to form polyvalent assemblages comprising 2-5 of the basic 4-chain units along with J chain. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (V_{H}) followed by three constant domains (C_{H}) for each of the α and γ chains and four C_{H} domains for μ and ε isotypes. Each L chain has at the N-terminus, a variable domain (V_{L}) followed by a constant domain (C_{L}) at its other end. The V_{L} is aligned with the V_{H} and the C_{L} is aligned with the first constant domain of the heavy chain (C_{H}1). Particular amino acid residues are believed to form an interface between the light chain and heavy chain variable domains. The pairing of a V_{H} and V_{L} together forms a single antigen-binding site. For the structure and properties of the different classes of antibodies, see, e.g., Basic and Clinical Immunology, 8th edition, Daniel P. Stites, Abba I. Terr and Tristram G. Parslow (eds.), Appleton & Lange, Norwalk, Conn., 1994, page 71, and Chapter 6.

The L chain from any vertebrate species can be assigned to one of two clearly distinct types, called kappa (κ) and lambda (λ), based on the amino acid sequences of their constant domains (C_{L}). Depending on the amino acid sequence of the constant domain of their heavy chains (C_{H}), immunoglobulins can be assigned to different classes or isotypes. There are five classes of immunoglobulins: IgA, IgD, IgE, IgG, and IgM, having heavy chains designated alpha (α), delta (δ), epsilon (ε), gamma (γ) and mu (μ), respectively. The γ and α classes are further divided into subclasses on the basis of relatively minor differences in C_{H} sequence and function, e.g., humans express the following subclasses: IgG1, IgG2, IgG3, IgG4, IgA1, and IgA2. The term “variable” refers to the fact that certain segments of the V domains differ extensively in sequence among antibodies. The V domain mediates antigen binding and defines specificity of a particular antibody for its particular antigen. However, the variability is not evenly distributed across the 110-amino acid span of the variable domains. Instead, the V regions consist of relatively invariant stretches called framework regions (FRs) of 15-30 amino acids separated by shorter regions of extreme variability called “hypervariable regions” that are each 9-12 amino acids long. The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC).

The term “hypervariable region” when used herein refers to the amino acid residues of an antibody that are responsible for antigen binding. The hypervariable region generally comprises amino acid residues from a “complementarity determining region” or “CDR” (e.g., around about residues 24-34 (L1), 50-56 (L2) and 89-97 (L3) in the V_{L}, and around about 31-35 (H1), 50-65 (H2) and 95-102 (H3) in the V_{H} when numbered in accordance with the Kabat numbering system; Kabat et al., Sequences of Proteins of Immunological Interest, 5th Ed. Public Health Service, National Institutes of Health, Bethesda, Md. (1991)); and/or those residues from a “hypervariable loop” (e.g., residues 24-34 (L1), 50-56 (L2) and 89-97 (L3) in the V_{L}, and 26-32 (H1), 52-56 (H2) and 95-101 (H3) in the V_{H} when numbered in accordance with the Chothia numbering system; Chothia and Lesk, J. Mol. Biol. 196:901-917 (1987)); and/or those residues from a “hypervariable loop”/CDR (e.g., residues 27-38 (L1), 56-65 (L2) and 105-120 (L3) in the V_{L}, and 27-38 (H1), 56-65 (H2) and 105-120 (H3) in the V_{H} when numbered in accordance with the IMGT numbering system; Lefranc, M. P. et al. Nucl. Acids Res. 27:209-212 (1999), Ruiz, M. e al. Nucl. Acids Res. 28:219-221 (2000)). Optionally the antibody has symmetrical insertions at one or more of the following points 28, 36 (L1), 63, 74-75 (L2) and 123 (L3) in the V_{L}, and 28, 36 (H1), 63, 74-75 (H2) and 123 (H3) in the V_{H} when numbered in accordance with AHo; Honneger, A. and Plunkthun, A. J. Mol. Biol. 309:657-670 (2001)).

By “germline nucleic acid residue” is meant the nucleic acid residue that naturally occurs in a germline gene encoding a constant or variable region. “Germline gene” is the DNA found in a germ cell (i.e., a cell destined to become an egg or in the sperm). A “germline mutation” refers to a heritable change in a particular DNA that has occurred in a germ cell or the zygote at the single-cell stage, and when transmitted to offspring, such a mutation is incorporated in every cell of the body. A germline mutation is in contrast to a somatic mutation which is acquired in a single body cell. In some cases, nucleotides in a germline DNA sequence encoding for a variable region are mutated (i.e., a somatic mutation) and replaced with a different nucleotide.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations that include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” is not to be construed as requiring production of the antibody by any particular method. For example, the monoclonal antibodies useful in the present invention may be prepared by the hybridoma methodology first described by Kohler et al., Nature, 256:495 (1975), or may be made using recombinant DNA methods in bacterial, eukaryotic animal or plant cells (see, e.g., U.S. Pat. No. 4,816,567). The “monoclonal antibodies” may also be isolated from phage antibody libraries using the techniques described in Clackson et al., Nature, 352:624-628 (1991) and Marks et al., J. Mol. Biol., 222:581-597 (1991), for example.

Monoclonal antibodies include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see U.S. Pat. No. 4,816,567; and Morrison et al., Proc. Natl. Acad. Sci. USA, 81:6851-6855 (1984)). Also provided are variable domain antigen-binding sequences derived from human antibodies. Accordingly, chimeric antibodies of primary interest herein include antibodies having one or more human antigen binding sequences (e.g., CDRs) and containing one or more sequences derived from a non-human antibody, e.g., an FR or C region sequence. In addition, chimeric antibodies of primary interest herein include those comprising a human variable domain antigen binding sequence of one antibody class or subclass and another sequence, e.g., FR or C region sequence, derived from another antibody class or subclass. Chimeric antibodies of interest herein also include those containing variable domain antigen-binding sequences related to those described herein or derived from a different species, such as a non-human primate (e.g., Old World Monkey, Ape, etc). Chimeric antibodies also include primatized and humanized antibodies.

Furthermore, chimeric antibodies may comprise residues that are not found in the recipient antibody or in the donor antibody. These modifications are made to further refine antibody performance. For further details, see Jones et al., Nature 321:522-525 (1986); Riechmann et al., Nature 332:323-329 (1988); and Presta, Curr. Op. Struct. Biol. 2:593-596 (1992).

A “humanized antibody” is generally considered to be a human antibody that has one or more amino acid residues introduced into it from a source that is non-human. These non-human amino acid residues are often referred to as “import” residues, which are typically taken from an “import” variable domain. Humanization is traditionally performed following the method of Winter and co-workers (Jones et al., Nature, 321:522-525 (1986); Reichmann et al., Nature, 332:323-327 (1988); Verhoeyen et al., Science, 239:1534-1536 (1988)), by substituting import hypervariable region sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species.

A “human antibody” is an antibody containing only sequences present in an antibody naturally produced by a human. However, as used herein, human antibodies may comprise residues or modifications not found in a naturally occurring human antibody, including those modifications and variant sequences described herein. These are typically made to further refine or enhance antibody performance.

An “intact” antibody is one that comprises an antigen-binding site as well as a C_{L} and at least heavy chain constant domains, C_{H} 1, C_{H} 2 and C_{H} 3. The constant domains may be native sequence constant domains (e.g., human native sequence constant domains) or amino acid sequence variant thereof. Preferably, the intact antibody has one or more effector functions. An “antibody fragment” comprises a portion of an intact antibody, preferably the antigen binding or variable region of the intact antibody. Examples of antibody fragments include Fab, Fab′, F(ab′)₂, and Fv fragments; diabodies; linear antibodies (see U.S. Pat. No. 5,641,870; Zapata et al., Protein Eng. 8(10): 1057-1062 [1995]); single-chain antibody molecules; and multispecific antibodies formed from antibody fragments.

The phrase “functional fragment or analog” of an antibody is a compound having qualitative biological activity in common with a full-length antibody. For example, a functional fragment or analog of an anti-IgE antibody is one that can bind to an IgE immunoglobulin in such a manner so as to prevent or substantially reduce the ability of such molecule from having the ability to bind to the high affinity receptor, Fc_{ε}RI.

Papain digestion of antibodies produces two identical antigen-binding fragments, called “Fab” fragments, and a residual “Fc” fragment, a designation reflecting the ability to crystallize readily. The Fab fragment consists of an entire L chain along with the variable region domain of the H chain (V_{H}), and the first constant domain of one heavy chain (C_{H} 1). Each Fab fragment is monovalent with respect to antigen binding, i.e., it has a single antigen-binding site. Pepsin treatment of an antibody yields a single large F(ab′)₂ fragment that roughly corresponds to two disulfide linked Fab fragments having divalent antigen-binding activity and is still capable of cross-linking antigen. Fab′ fragments differ from Fab fragments by having additional few residues at the carboxy terminus of the C_{H}1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments that have hinge cysteines between them. Other chemical couplings of antibody fragments are also known.

The “Fc” fragment comprises the carboxy-terminal portions of both H chains held together by disulfides. The effector functions of antibodies are determined by sequences in the Fc region, which region is also the part recognized by Fc receptors (FcR) found on certain types of cells.

“Fv” is the minimum antibody fragment that contains a complete antigen-recognition and -binding site. This fragment consists of a dimer of one heavy- and one light-chain variable region domain in tight, non-covalent association. From the folding of these two domains emanate six hypervariable loops (three loops each from the H and L chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

“Single-chain Fv” also abbreviated as “sFv” or “scFv” are antibody fragments that comprise the V_{H} and V_{L} antibody domains connected into a single polypeptide chain. Preferably, the sFv polypeptide further comprises a polypeptide linker between the V_{H} and V_{L} domains that enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994); Borrebaeck 1995, infra.

The term “diabodies” refers to small antibody fragments prepared by constructing sFv fragments (see preceding paragraph) with short linkers (about 5-10 residues) between the V_{H} and V_{L} domains such that inter-chain but not intra-chain pairing of the V domains is achieved, resulting in a bivalent fragment, i.e., fragment having two antigen-binding sites. Bispecific diabodies are heterodimers of two “crossover” sFv fragments in which the V_{H} and V_{L} domains of the two antibodies are present on different polypeptide chains. Diabodies are described more fully in, for example, EP 404,097; WO 93/11161; and Hollinger et al., Proc. Natl. Acad. Sci. USA, 90:6444-6448 (1993).

As used herein, an antibody that “internalizes” is one that is taken up by (i.e., enters) the cell upon binding to an antigen on a mammalian cell (e.g., a cell surface polypeptide or receptor). The internalizing antibody will of course include antibody fragments, human or chimeric antibody, and antibody conjugates. For certain therapeutic applications, internalization in vivo is contemplated. The number of antibody molecules internalized will be sufficient or adequate to kill a cell or inhibit its growth, especially an infected cell. Depending on the potency of the antibody or antibody conjugate, in some instances, the uptake of a single antibody molecule into the cell is sufficient to kill the target cell to which the antibody binds. For example, certain toxins are highly potent in killing such that internalization of one molecule of the toxin conjugated to the antibody is sufficient to kill the infected cell.

As used herein, an antibody is said to be “immunospecific,” “specific for” or to “specifically bind” an antigen if it reacts at a detectable level with the antigen, preferably with an affinity constant, Kₐ, of greater than or equal to about 10⁴ M⁻¹, or greater than or equal to about 10⁵ M⁻¹, greater than or equal to about 10⁶ M⁻¹, greater than or equal to about 10⁷ M⁻¹, or greater than or equal to 10⁸ M⁻¹. Affinity of an antibody for its cognate antigen is also commonly expressed as a dissociation constant K_{D}, and in certain embodiments, HuM2e antibody specifically binds to M2e if it binds with a K_{D} of less than or equal to 10⁻⁴ M, less than or equal to about 10⁻⁵ M, less than or equal to about 10⁻⁶ M, less than or equal to 10⁻⁷ M, or less than or equal to 10⁻⁸ M. Affinities of antibodies can be readily determined using conventional techniques, for example, those described by Scatchard et al. (Ann. N.Y. Acad. Sci. USA 51:660 (1949)).

Binding properties of an antibody to antigens, cells or tissues thereof may generally be determined and assessed using immunodetection methods including, for example, immunofluorescence-based assays, such as immuno-histochemistry (IHC) and/or fluorescence-activated cell sorting (FACS).

An antibody having a “biological characteristic” of a designated antibody is one that possesses one or more of the biological characteristics of that antibody which distinguish it from other antibodies. For example, in certain embodiments, an antibody with a biological characteristic of a designated antibody will bind the same epitope as that bound by the designated antibody and/or have a common effector function as the designated antibody. The term “antagonist” antibody is used in the broadest sense, and includes an antibody that partially or fully blocks, inhibits, or neutralizes a biological activity of an epitope, polypeptide, or cell that it specifically binds. Methods for identifying antagonist antibodies may comprise contacting a polypeptide or cell specifically bound by a candidate antagonist antibody with the candidate antagonist antibody and measuring a detectable change in one or more biological activities normally associated with the polypeptide or cell.

Antibody “effector functions” refer to those biological activities attributable to the Fc region (a native sequence Fc region or amino acid sequence variant Fc region) of an antibody, and vary with the antibody isotype. Examples of antibody effector functions include: C1q binding and complement dependent cytotoxicity; Fc receptor binding; antibody-dependent cell-mediated cytotoxicity (ADCC); phagocytosis; down regulation of cell surface receptors (e.g., B cell receptor); and B cell activation.

By “binding to” a molecule is meant having a physicochemical affinity for that molecule.

By “control” or “reference” is meant a standard of comparison. In one aspect, as used herein, “changed as compared to a control” sample or subject is understood as having a level that is statistically different than a sample from a normal, untreated, or control sample. Control samples include, for example, cells in culture, one or more laboratory test animals, or one or more human subjects. Methods to select and test control samples are within the ability of those in the art. An analyte can be a naturally occurring substance that is characteristically expressed or produced by the cell or organism (e.g., an antibody, a protein) or a substance produced by a reporter construct (e.g, β-galactosidase or luciferase). Depending on the method used for detection, the amount and measurement of the change can vary. Determination of statistical significance is within the ability of those skilled in the art, e.g., the number of standard deviations from the mean that constitute a positive result.

“Detect” refers to identifying the presence, absence, or amount of the agent (e.g., a nucleic acid molecule, for example deoxyribonucleic acid (DNA) or ribonucleic acid (RNA)) to be detected.

By “detectable label” is meant a composition that when linked (e.g., joined—directly or indirectly) to a molecule of interest renders the latter detectable, via, for example, spectroscopic, photochemical, biochemical, immunochemical, or chemical means. Direct labeling can occur through bonds or interactions that link the label to the molecule, and indirect labeling can occur through the use of a linker or bridging moiety which is either directly or indirectly labeled. Bridging moieties may amplify a detectable signal. For example, useful labels may include radioactive isotopes, magnetic beads, metallic beads, colloidal particles, fluorescent labeling compounds, electron-dense reagents, enzymes (for example, as commonly used in an enzyme-linked immunosorbent assay (ELISA)), biotin, digoxigenin, or haptens. When the fluorescently labeled molecule is exposed to light of the proper wave length, its presence can then be detected due to fluorescence. Among the most commonly used fluorescent labeling compounds are fluorescein isothiocyanate, rhodamine, phycoerythrin, phycocyanin, allophycocyanin, p-phthaldehyde and fluorescamine. The molecule can also be detectably labeled using fluorescence emitting metals such as 152 Eu, or others of the lanthanide series. These metals can be attached to the molecule using such metal chelating groups as diethylenetriaminepentacetic acid (DTPA) or ethylenediaminetetraacetic acid (EDTA). The molecule also can be detectably labeled by coupling it to a chemiluminescent compound. The presence of the chemiluminescent-tagged molecule is then determined by detecting the presence of luminescence that arises during the course of chemical reaction. Examples of particularly useful chemiluminescent labeling compounds are luminol, isoluminol, theromatic acridinium ester, imidazole, acridinium salt and oxalate ester.

A “detection step” may use any of a variety of known methods to detect the presence of nucleic acid (e.g., methylated DNA) or polypeptide. The types of detection methods in which probes can be used include Western blots, Southern blots, dot or slot blots, and Northern blots.

As used herein, the term “diagnosing” refers to classifying pathology or a symptom, determining a severity of the pathology (e.g., grade or stage), monitoring pathology progression, forecasting an outcome of pathology, and/or determining prospects of recovery.

By the terms “effective amount” and “therapeutically effective amount” of a formulation or formulation component is meant a sufficient amount of the formulation or component, alone or in a combination, to provide the desired effect. For example, by “an effective amount” is meant an amount of a compound, alone or in a combination, required to ameliorate the symptoms of a disease, e.g., melanoma, relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

The term “expression profile” is used broadly to include a genomic expression profile. Profiles may be generated by any convenient means for determining a level of a nucleic acid sequence, e.g., quantitative hybridization of microRNA, labeled microRNA, amplified microRNA, complementary/synthetic DNA (cDNA), etc., quantitative polymerase chain reaction (PCR), and ELISA for quantitation, and allow the analysis of differential gene expression between two samples. A subject or patient tumor sample is assayed. Samples are collected by any convenient method, as known in the art. According to some embodiments, the term “expression profile” means measuring the relative abundance of the nucleic acid sequences in the measured samples.

By “FDR” is meant False Discovery Rate. When performing multiple statistical tests, for example, in comparing the signal of two groups in multiple data features, there is an increasingly high probability of obtaining false positive results, by random differences between the groups that can reach levels that would otherwise be considered statistically significant. In some cases, in order to limit the proportion of such false discoveries, statistical significance is defined only for data features in which the differences reached a p-value (by two-sided t-test) below a threshold, which is dependent on the number of tests performed and the distribution of p-values obtained in these tests.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. For example, a fragment may contain 10, 20, 30, 40, 50, 60, 70, 80, 90, or 100, 200, 300, 400, 500, 600, 700, 800, 900, or 1000 nucleotides or amino acids. However, the invention also comprises polypeptides and nucleic acid fragments, so long as they exhibit the desired biological activity of the full length polypeptides and nucleic acid, respectively. A nucleic acid fragment of almost any length is employed. For example, illustrative polynucleotide segments with total lengths of about 10,000, about 5000, about 3000, about 2,000, about 1,000, about 500, about 200, about 100, about 50 base pairs in length (including all intermediate lengths) are included in many implementations of this invention. Similarly, a polypeptide fragment of almost any length is employed. For example, illustrative polypeptide segments with total lengths of about 10,000, about 5,000, about 3,000, about 2,000, about 1,000, about 5,000, about 1,000, about 500, about 200, about 100, or about 50 amino acids in length (including all intermediate lengths) are included in many implementations of this invention.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation.

A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

Similarly, by “substantially pure” is meant a nucleotide or polypeptide that has been separated from the components that naturally accompany it. Typically, the nucleotides and polypeptides are substantially pure when they are at least 60%, 70%, 80%, 90%, 95%, or even 99%, by weight, free from the proteins and naturally-occurring organic molecules with they are naturally associated.

By “isolated nucleic acid” is meant a nucleic acid that is free of the genes which flank it in the naturally-occurring genome of the organism from which the nucleic acid is derived. The term covers, for example: (a) a DNA which is part of a naturally occurring genomic DNA molecule, but is not flanked by both of the nucleic acid sequences that flank that part of the molecule in the genome of the organism in which it naturally occurs; (b) a nucleic acid incorporated into a vector or into the genomic DNA of a prokaryote or eukaryote in a manner, such that the resulting molecule is not identical to any naturally occurring vector or genomic DNA; (c) a separate molecule such as a synthetic cDNA, a genomic fragment, a fragment produced by polymerase chain reaction (PCR), or a restriction fragment; and (d) a recombinant nucleotide sequence that is part of a hybrid gene, i.e., a gene encoding a fusion protein. Isolated nucleic acid molecules according to the present invention further include molecules produced synthetically, as well as any nucleic acids that have been altered chemically and/or that have modified backbones. For example, the isolated nucleic acid is a purified cDNA or RNA polynucleotide. Isolated nucleic acid molecules also include messenger ribonucleic acid (mRNA) molecules.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

The term “immobilized” or “attached” refers to a probe (e.g., nucleic acid or protein) and a solid support in which the binding between the probe and the solid support is sufficient to be stable under conditions of binding, washing, analysis, and removal. The binding may be covalent or non-covalent. Covalent bonds may be formed directly between the probe and the solid support or may be formed by a cross linker or by inclusion of a specific reactive group on either the solid support or the probe or both molecules. Non-covalent binding may be one or more of electrostatic, hydrophilic, and hydrophobic interactions. Included in non-covalent binding is the covalent attachment of a molecule to the support and the non-covalent binding of a biotinylated probe to the molecule. Immobilization may also involve a combination of covalent and non-covalent interactions.

By “marker” is meant any protein or polynucleotide having an alteration in expression level or activity that is associated with a disease or disorder, e.g., melanoma.

By “melanoma-associated gene” is meant a nucleic acid associated with the pathogenesis of melanoma.

By “modulate” is meant alter (increase or decrease). Such alterations are detected by standard art-known methods such as those described herein.

The term, “normal amount” refers to a normal amount of a complex in an individual known not to be diagnosed with melanoma. The amount of the molecule can be measured in a test sample and compared to the “normal control level,” utilizing techniques such as reference limits, discrimination limits, or risk defining thresholds to define cutoff points and abnormal values (e.g., for melanoma). The “normal control level” means the level of one or more proteins (or nucleic acids) or combined protein indices (or combined nucleic acid indices) typically found in a subject known not to be suffering from melanoma. Such normal control levels and cutoff points may vary based on whether a molecule is used alone or in a formula combining other proteins into an index. Alternatively, the normal control level can be a database of protein patterns from previously tested subjects who did not convert to melanoma over a clinically relevant time horizon. In another aspect, the normal control level can be a level relative to a housekeeping gene.

The level that is determined may be the same as a control level or a cut off level or a threshold level, or may be increased or decreased relative to a control level or a cut off level or a threshold level. In some aspects, the control subject is a matched control of the same species, gender, ethnicity, age group, smoking status, body mass index (BMI), current therapeutic regimen status, medical history, or a combination thereof, but differs from the subject being diagnosed in that the control does not suffer from the disease in question or is not at risk for the disease.

Relative to a control level, the level that is determined may be an increased level. As used herein, the term “increased” with respect to level (e.g., expression level, biological activity level, etc.) refers to any % increase above a control level. The increased level may be at least or about a 1% increase, at least or about a 5% increase, at least or about a 10% increase, at least or about a 15% increase, at least or about a 20% increase, at least or about a 25% increase, at least or about a 30% increase, at least or about a 35% increase, at least or about a 40% increase, at least or about a 45% increase, at least or about a 50% increase, at least or about a 55% increase, at least or about a 60% increase, at least or about a 65% increase, at least or about a 70% increase, at least or about a 75% increase, at least or about a 80% increase, at least or about a 85% increase, at least or about a 90% increase, or at least or about a 95% increase, relative to a control level.

Relative to a control level, the level that is determined may be a decreased level. As used herein, the term “decreased” with respect to level (e.g., expression level, biological activity level, etc.) refers to any % decrease below a control level. The decreased level may be at least or about a 1% decrease, at least or about a 5% decrease, at least or about a 10% decrease, at least or about a 15% decrease, at least or about a 20% decrease, at least or about a 25% decrease, at least or about a 30% decrease, at least or about a 35% decrease, at least or about a 40% decrease, at least or about a 45% decrease, at least or about a 50% decrease, at least or about a 55% decrease, at least or about a 60% decrease, at least or about a 65% decrease, at least or about a 70% decrease, at least or about a 75% decrease, at least or about a 80% decrease, at least or about a 85% decrease, at least or about a 90% decrease, or at least or about a 95% decrease, relative to a control level.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity, e.g., at least 80%, at least 85%, at least 90%, at least 95%, or at least 99% identity. Polynucleotides having “substantial identity” to an endogenous sequence are typically capable of hybridizing with at least one strand of a double-stranded nucleic acid molecule.

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “neoplasia” is meant a disease or disorder characterized by excess proliferation or reduced apoptosis. Illustrative neoplasms for which the invention can be used include, but are not limited to pancreatic cancer, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, nile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, glioblastoma multiforme, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma).

As used herein, in one aspect, “next-generation sequencing” (NGS), also known as high-throughput sequencing, is the catch-all term used to describe a number of different sequencing methodologies including, but not limited to, Illumina® sequencing, Roche 454 Sequencing™, Ion Torrent™: Proton/personal genome machine (PGM) sequencing, and SOLiD sequencing. These recent technologies allow for sequencing DNA and RNA much more quickly and cheaply than the previously used Sanger sequencing. See, LeBlanc et al., 2015 Cancers, 7: 1925-1958, incorporated herein by reference; and Goodwin et al., 2016 Nature Reviews Genetics, 17: 333-351, incorporated herein by reference.

As used herein, “obtaining” as in “obtaining an agent” includes synthesizing, purchasing, or otherwise acquiring the agent.

Unless specifically stated or obvious from context, as used herein, the term “or” is understood to be inclusive. Unless specifically stated or obvious from context, as used herein, the terms “a”, “an”, and “the” are understood to be singular or plural.

The phrase “pharmaceutically acceptable carrier” is art recognized and includes a pharmaceutically acceptable material, composition or vehicle, suitable for administering compounds of the present invention to mammals. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

By “protein” or “polypeptide” or “peptide” is meant any chain of more than two natural or unnatural amino acids, regardless of post-translational modification (e.g., glycosylation or phosphorylation), constituting all or part of a naturally-occurring or non-naturally occurring polypeptide or peptide, as is described herein.

“Primer set” means a set of oligonucleotides that may be used, for example, for PCR. A primer set would consist of at least 2, 4, 6, 8, 10, 12, 14, 16, 18, 20, 30, 40, 50, 60, 80, 100, 200, 250, 300, 400, 500, 600, or more primers.

The terms “preventing” and “prevention” refer to the administration of an agent or composition to a clinically asymptomatic individual who is at risk of developing, susceptible, or predisposed to a particular adverse condition, disorder, or disease, and thus relates to the prevention of the occurrence of symptoms and/or their underlying cause.

The term “prognosis,” “staging,” and “determination of aggressiveness” are defined herein as the prediction of the degree of severity of the neoplasia, e.g., melanoma, and of its evolution as well as the prospect of recovery as anticipated from usual course of the disease. Once the aggressiveness (e.g. the Gleason score) has been determined, appropriate methods of treatments are chosen.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it is understood that the particular value forms another aspect. It is further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. It is also understood that throughout the application, data are provided in a number of different formats and that this data represent endpoints and starting points and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

By “reduces” is meant a negative alteration of at least 10%, 25%, 50%, 75%, or 100%.

A “reference sequence” is a defined sequence used as a basis for sequence comparison or a gene expression comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 40 nucleotides, preferably at least about 60 nucleotides, more preferably at least about 75 nucleotides, and even more preferably about 100 nucleotides or about 300 or about 500 nucleotides or any integer thereabout or there between.

The term “sample” as used herein refers to a biological sample obtained for the purpose of evaluation in vitro. Exemplary tissue samples for the methods described herein include tissue samples from melanoma tumors or the surrounding microenvironment (i.e., the stroma). With regard to the methods disclosed herein, the sample or patient sample preferably may comprise any body fluid or tissue. In some embodiments, the bodily fluid includes, but is not limited to, blood, plasma, serum, lymph, breast milk, saliva, mucous, semen, vaginal secretions, cellular extracts, inflammatory fluids, cerebrospinal fluid, feces, vitreous humor, or urine obtained from the subject. In some aspects, the sample is a composite panel of at least two of a blood sample, a plasma sample, a serum sample, and a urine sample. In exemplary aspects, the sample comprises blood or a fraction thereof (e.g., plasma, serum, fraction obtained via leukopheresis). Preferred samples are whole blood, serum, plasma, or urine. A sample can also be a partially purified fraction of a tissue or bodily fluid.

A reference sample can be a “normal” sample, from a donor not having the disease or condition fluid, or from a normal tissue in a subject having the disease or condition. A reference sample can also be from an untreated donor or cell culture not treated with an active agent (e.g., no treatment or administration of vehicle only). A reference sample can also be taken at a “zero time point” prior to contacting the cell or subject with the agent or therapeutic intervention to be tested or at the start of a prospective study.

A “solid support” describes a strip, a polymer, a bead, or a nanoparticle. The strip may be a nucleic acid-probe (or protein) coated porous or non-porous solid support strip comprising linking a nucleic acid probe to a carrier to prepare a conjugate and immobilizing the conjugate on a porous solid support. Well-known supports or carriers include glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of the present invention. The support material may have virtually any possible structural configuration so long as the coupled molecule is capable of binding to a binding agent (e.g., an antibody or nucleic acid molecule). Thus, the support configuration may be spherical, as in a bead, or cylindrical, as in the inside surface of a test tube, or the external surface of a rod. Alternatively, the surface may be flat such as a sheet, or test strip, etc. For example, the supports include polystyrene beads. Those skilled in the art will know many other suitable carriers for binding antibody or antigen, or will be able to ascertain the same by use of routine experimentation. In other aspects, the solid support comprises a polymer, to which an agent is chemically bound, immobilized, dispersed, or associated. A polymer support may be a network of polymers, and may be prepared in bead form (e.g., by suspension polymerization). The location of active sites introduced into a polymer support depends on the type of polymer support. For example, in a swollen-gel-bead polymer support the active sites are distributed uniformly throughout the beads, whereas in a macroporous-bead polymer support they are predominantly on the internal surfaces of the macropores. The solid support, e.g., a device contains a binding agent alone or together with a binding agent for at least one, two, three or more other molecules.

By “specifically binds” is meant a compound or antibody that recognizes and binds a polypeptide of the invention, but which does not substantially recognize and bind other molecules in a sample, for example, a biological sample, which naturally includes a polypeptide of the invention.

A “specific binding agent” describes agents having greater than 10-fold, preferably greater than 100-fold, and most preferably, greater than 1000-fold affinity for the target molecule as compared to another molecule. As the skilled artisan will appreciate the term specific is used to indicate that other biomolecules present in the sample do not significantly bind to the binding agent specific for the target molecule. Preferably, the level of binding to a biomolecule other than the target molecule results in a binding affinity which is at most only 10% or less, only 5% or less only 2% or less or only 1% or less of the affinity to the target molecule, respectively. A preferred specific binding agent will fulfill both the above minimum criteria for affinity as well as for specificity. For example, an antibody has a binding affinity in the low micromolar (10⁻⁶), nanomolar (10⁻⁷-10⁻⁹), with high affinity antibodies in the low nanomolar (10⁻⁹) or pico molar (10⁻¹²) range for its specific target molecule.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, at least 70%, at least 80%, at least 85%, at least 90%, at least 95%, or at least 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

The term “subject” as used herein includes all members of the animal kingdom prone to suffering from the indicated disorder. In some aspects, the subject is a mammal, and in some aspects, the subject is a human. The methods are also applicable to companion animals such as dogs and cats as well as livestock such as cows, horses, sheep, goats, pigs, and other domesticated and wild animals.

A subject “suffering from or suspected of suffering from” a specific disease, condition, or syndrome has a sufficient number of risk factors or presents with a sufficient number or combination of signs or symptoms of the disease, condition, or syndrome such that a competent individual would diagnose or suspect that the subject was suffering from the disease, condition, or syndrome. Methods for identification of subjects suffering from or suspected of suffering from conditions associated with cancer (e.g., melanoma) is within the ability of those in the art. Subjects suffering from, and suspected of suffering from, a specific disease, condition, or syndrome are not necessarily two distinct groups.

As used herein, “susceptible to” or “prone to” or “predisposed to” or “at risk of developing” a specific disease or condition refers to an individual who based on genetic, environmental, health, and/or other risk factors is more likely to develop a disease or condition than the general population. An increase in likelihood of developing a disease may be an increase of about 10%, 20%, 50%, 100%, 150%, 200%, or more.

The terms “treating” and “treatment” as used herein refer to the administration of an agent or formulation to a clinically symptomatic individual afflicted with an adverse condition, disorder, or disease, so as to effect a reduction in severity and/or frequency of symptoms, eliminate the symptoms and/or their underlying cause, and/or facilitate improvement or remediation of damage. It will be appreciated that, although not precluded, treating a disorder or condition does not require that the disorder, condition or symptoms associated therewith be completely eliminated.

As used herein, in one aspect, the “tumor microenvironment” (TME) is the cellular environment in which a tumor exists, including surrounding blood vessels, immune cells, fibroblasts, bone marrow-derived inflammatory cells, lymphocytes, signaling molecules and the extracellular matrix (ECM). The tumor and the surrounding microenvironment are closely related and interact constantly. Tumors can influence the microenvironment by releasing extracellular signals, promoting tumor angiogenesis and inducing peripheral immune tolerance, while the immune cells in the microenvironment can affect the growth and evolution of cancerous cells, such as in immuno-editing.

In some cases, a composition of the invention is administered orally or systemically. Other modes of administration include rectal, topical, intraocular, buccal, intravaginal, intracisternal, intracerebroventricular, intratracheal, nasal, transdermal, within/on implants, or parenteral routes. The term “parenteral” includes subcutaneous, intrathecal, intravenous, intramuscular, intraperitoneal, or infusion. Intravenous or intramuscular routes are not particularly suitable for long-term therapy and prophylaxis. They could, however, be preferred in emergency situations. Compositions comprising a composition of the invention can be added to a physiological fluid, such as blood. Oral administration can be preferred for prophylactic treatment because of the convenience to the patient as well as the dosing schedule. Parenteral modalities (subcutaneous or intravenous) may be preferable for more acute illness, or for therapy in patients that are unable to tolerate enteral administration due to gastrointestinal intolerance, ileus, or other concomitants of critical illness. Inhaled therapy may be most appropriate for pulmonary vascular diseases (e.g., pulmonary hypertension).

Pharmaceutical compositions may be assembled into kits or pharmaceutical systems for use in arresting cell cycle in rapidly dividing cells, e.g., cancer cells. Kits or pharmaceutical systems according to this aspect of the invention comprise a carrier means, such as a box, carton, tube, having in close confinement therein one or more container means, such as vials, tubes, ampoules, bottles, syringes, or bags. The kits or pharmaceutical systems of the invention may also comprise associated instructions for using the kit.

Any compositions or methods provided herein can be combined with one or more of any of the other compositions and methods provided herein.

Any compositions or methods provided herein can be combined with one or more of any of the other compositions and methods provided herein.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

Other features and advantages of the invention will be apparent from the following description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All published foreign patents and patent applications cited herein are incorporated herein by reference. Genbank and NCBI submissions indicated by accession number cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

### BRIEF DESCRIPTION OF THE DRAWINGS

FIG. 1A-FIG. 1C is a series of graphs showing transcriptomic signatures of resistance to CTLA4 blockade. FIG. 1A is a volcano plot depicting 975 genes enriched in NB tumors and 428 enriched genes in CB tumors. Relative positions of Xq28-CGAs, miR-211, TRPM1, and immune-related genes are shown. FIG. 1B is a graph showing a 75 Kb region within the Xq28 locus containing the 8 CGAs. Bar plots depicting the individual fold changes for each MAGE-A and CSAG gene within this locus are shown for RNA seq in discovery cohort (FIG. 1B (top)) and independent cohort (FIG. 1B (middle)). FIG. 1B (bottom) shows qPCR validation of expression of Xq28-CGA genes in benefit (n=10) and no-benefit (n=11) samples; fold changes are shown relative to HPRT1 gene. FIG. 1C is a graph showing that genes co-enriched with Xq28-CGA expression in TCGA significantly overlap with genes associated with NB tumors.

FIG. 2A is a volcano plot depicting genes enriched in ‘no progressive disease’ (No PD) and ‘progressive disease’ (PD) groups at week 13 in the ipilimumab-nivolumab arm of the CheckMate 064 trial (Weber et al., 2016) (validation cohort). FIG. 2B is a series of box plots depicting RNA-seq expression for CRMA MAGEA genes between the No PD and PD groups in validation cohort. FIG. 2C is a bar graph showing IHC staining analysis of MAGE-A protein expression in pre-ipilimumab melanoma biopsies. FIG. 2D is a photomicrograph showing examples of MAGE-A protein expression from a patient in the CB (left) and NB (right) groups. Magnification, ×1000.

FIG. 3A-FIG. 3B is a series of graphs that show DNA methylation patterns in resistant tumors and Xq28-high TCGA samples. FIG. 3A shows that MAGEA3 and MAGEA6 promoters in ‘no benefit’ patients (n=3) are preferentially de-methylated compared to ‘clinical benefit’ patients (n=3) as validated by bisulfite PCR. The plot highlights the local regression (solid line) of the mean methylation for every CpG (dots) along the MAGEA3 and MAGEA6 promoter in CB versus NB patients. The standard deviation is indicated by the shaded area. Note that both promoter sequences are identical within the analyzed amplicon span. FIG. 3B is a volcano plot of differentially methylated probes with false discovery rate <0.05 across the genome between Xq28 low and Xq28 high expression groups in the TCGA melanoma cohort.

FIG. 4A-FIG. 4E is a series of graphs showing transcriptomic signatures of clinical benefit to CTLA4 blockade. FIG. 4A is a series of bar plots depicting the individual fold changes for miR-211, TRPM1, and other TRPM family members. FIG. 4B is a bar graph of percentage of differentially expressed genes related to immune response, as determined by self-curation in both NB and CB tumors. FIG. 4C is a graph showing that genes associated with clinical benefit significantly overlap with genes co-enriched with miR-211 expression in TCGA but not with genes inversely associated with miR-211 expression. FIG. 4D is a graph showing that miR-211, miR-185 and miR-513A2 are significantly upregulated in clinical benefit tumors. FIG. 4E is a graph that shows proliferative gene expression signatures are significantly enriched in clinical benefit tumors, while the invasive gene expression signatures are significantly enriched in no benefit tumors.

FIG. 5A-FIG. 5B are a series of graphs showing that molecular signatures of outcome to CTLA4 blockade are unique and do not predict outcome to PD1 blockade. Specifically, FIG. 5A and FIG. 5B are barplots of fold changes for individual Xq28-CGAs, miR-211 and TRPM1 in pretreatment melanoma samples from responding and nonresponding patients to PD1 blockade. FIG. 5C is a series of box plots of RNA-seq expression values for each MAGEA gene in the CRMA locus within no progressive disease (no PD; blue) (n=23) and progressive disease (PD; orange) (n=14) pre-nivolumab melanoma tumors from CheckMate 064 (Weber et al., 2016).

FIG. 6A-FIG. 6C is a series of graphs showing that Xq28-CGA antigen and miR-211 predict outcome to CTLA4 blockade. FIG. 6A is a graph showing correlation of Xq28 CT antigen and miR-211 expression for all 40 patients in discovery cohort including NB, CB and “long term survival with no clinical benefit.” FIG. 6B is a graph showing ROC analysis comparing neoantigen load, CTLA4 expression, combined neoantigen load+CTLA4 expression, combined “Xq28-CGA+miR-211” expression, and a combination of all four parameters for all patients. FIG. 6C is a graph showing overall survival Kaplan-Meier curves for combination “neoantigen load+CTLA4 (left),” “Xq28-CGA+miR-211 (middle)” and “Xq28-CGA+miR-211+neoantigen load+CTLA4 (right).” FIG. 6D is Kaplan-Meier overall survival analysis comparing patients from discovery cohort classified by expression of MAGE-A protein. FIG. 6E is a table showing the Cox proportional hazards model of risk factors for outcomes after ipilimumab therapy. FIG. 6F is a graph showing the Kaplan-Meier overall survival analysis of TCGA melanoma samples with high or low expression of Xq28-CGA genes.

FIG. 7 is a series of graphs showing qPCR validation of Xq28-CGA genes using different housekeeping genes. Barplots showing similar fold changes of Xq28 genes from qPCR using either GAPDH (top) or PGK1 (bottom) as housekeeping genes.

FIG. 8 a series of graphs showing copy number analysis of Xq28 region in Clinical Benefit/No Benefit patients. Neither the locus average of copy ratios, nor copy ratios on individual targets showed a statistically significant germline or somatic variation between the two groups at 5% level.

FIG. 9 is a bar graph showing that DTIC/temozolamide treatment history does not affect outcome after ipilimumab. Patients were grouped into “DTIC” cohort if DTIC or temozolamide were used as treatment any timepoint before ipilimumab.

FIG. 10A-FIG. 10B is a series of graphs showing that gender and purity do not affect outcome after ipilimumab. Barplots of gender and purity comparing CB and NB groups showing no effect.

FIG. 11A-FIG. 11D is a series of graphs that show that MAGE-A proteins may degrade the danger molecule HMGB1. FIG. 11A is a bar graph showing the results of an in vitro screen for MAGE-TRIM28 ubiquitination substrates identifies HMGB1 (p<0.05). FIG. 11B is a series of photomicrographs with immunofluorescence staining for MAGE-A and HMGB1 showing mutual exclusion in five patient samples from the discovery cohort in addition to a human xenograft melanoma. Magnification ×400. FIG. 11C is a bar graph showing the percentage of differentially expressed genes related to immune response, in both NB (3.5%) and CB (56%) tumors. FIG. 11D is a graph showing gene set enrichment analysis of immune gene sets showing p-value of enrichment (signed according to enrichment score). Dashed line represents p=0.05.

FIG. 12A-FIG. 12D is a series of graphs showing that Xq28-CGA genes are upregulated in ipilimumab-resistant melanoma samples. FIG. 12A is a heatmap showing relative expression of Xq28-CGA genes for CB and NB patients in the discovery cohort with annotations for gender, purity and RECIST response. FIG. 12B is a heatmap showing relative expression of Xq28-CGA genes in the validation set (CheckMate 064) with annotations for gender and RECIST response. FIG. 12C is a series of box plots depicting the individual fold changes for each MAGEA gene within the Xq28-CGA locus for patients with no progressive (“no PD”) or progressive disease (“PD”) in the discovery cohort; p-values using the Wilcoxon test.

FIG. 13A is a graph showing that bisulfite PCR of unique methylation sites within the gene bodies of MAGEA3/A6/A12 genes highlights a slight to moderate decrease in methylation in NB patients (n=4, orange) vs. CB patients (n=4, blue). The position of 3 PCR amplicons are highlighted, and the plots highlight the mean methylation for each CpG within the amplicon region. FIG. 13B is a graph showing chromosomal locations of 65,467 hypomethylated (top) and 47 hypermethylated (bottom) probes in “CRMA-high” TCGA melanoma samples.

FIG. 14 is a graph showing biological processes enriched in NB transcriptomes. Specifically, FIG. 14 is a heatmap showing relative gene expression of NB-enriched biological categories (see Table 2) along with lack of enrichment of NY-ESO-1 and melanoma differentiation antigens. Annotations of gender, purity and RECIST response included.

FIG. 15A and FIG. 15B are graphs of immunofluorescence staining on a melanoma tissue mnicroarray (TMA) comprising 100 samples (9 benign nevi tumors, 91 primary and metastatic melanomas) using antibodies against MAGE and HMG3B. The fraction of HMGB1 positive cells were comparable in MAGE negative cells from the benign nevi and malignant tumors, but was significantly reduced in cells from MAGE+ malignant samples ((26% and 31% vs 8%, Chi-square test p<2.2×10⁻¹⁶ FIG. 15A). FIG. 15B is a graph showing that in 13 out of 15 melanomas (stained on the TMA) that had any MAGE positive cells, at least 85% of MAGE+ cells lack HMGB1 (FIG. 15B).

FIG. 16 is a graph showing the significant overexpression of genes, TLR9 and IL12A, in the clinical benefit tumors. These genes are downstream of the HMGB1 pathway and show this pathway is activated.

FIG. 17A is a graph showing significantly decreased autophagy (indicated by LC3B positive staining) in MAGE-A+ melanomas on a tissue microarray by immunohistochemistry. FIG. 17B is a graph showing significantly impaired autophagy (indicated by absent LC3B staining or double-positive LC3B/p62 staining) in MAGE-A+ melanomas.

### DETAILED DESCRIPTION OF THE INVENTION

The invention is based, at least in part, upon the identification of a gene expression signature that discriminates clinical outcomes of CTLA4 blockade. CTLA4 blockade can induce durable clinical remissions in a minority of patients with metastatic melanoma. However, prior to the invention described herein, molecular signatures precisely predicting response and resistance were unknown. While increased neoantigen burden and clonality as well as increased expression of immune-related genes correlate with response, prior to the invention described herein, these molecular signatures were not clinically robust. Moreover, mechanistic insight into clinical resistance was lacking, and prior to the invention described herein, the contribution of epigenetic mechanisms was poorly understood.

Ipilimumab is an FDA-approved antibody targeting the CTLA4 pathway. Ipilimumab was the first agent to show an overall survival benefit in metastatic melanoma. However, only 15-20% of patients benefit from ipilimumab treatment. Prior to the invention described herein, there was no way to predict clinical outcome. Because ipilimumab carries significant autoimmune toxicity, predicting who will and will not benefit is of critical clinical importance. Ipilimumab is falling out of clinical use with the approval of newer, less toxic immunotherapies; however, long term survival data is only available for this agent. Thus, the results presented herein allow for precisely pairing CTLA4 blockade therapy with the appropriate patient.

Briefly, transcriptomic and clinical data from three independent melanoma cohorts were analyzed to identify correlates of outcome to CTLA4 blockade: (i) Cohort 1 comprised 40 pre-ipilimumab tumor samples (discovery set); (ii) Cohort 2 comprised 6 pre-treatment (ipilimumab or tremelimumab) samples (validation set 1); and (iii) Cohort 3 comprised 473 melanoma samples from The Cancer Genome Atlas (validation set 2). As described herein, genes that were differentially expressed between the clinical benefit (CB) and no clinical benefit (NB) groups were identified using unadjusted Wilcoxon tests (p<0.05), and a two-fold over-expression threshold in either group. The hypergeometric test was used to evaluate overlap of differentially expressed genes between cohorts, and the single-sample gene set enrichment (ssGSEA) method to identify pathway-level differences. Moreover, TCGA melanoma samples with high expression of the Xq28 CGA locus exhibited profound global hypomethylation, implicating epigenomic dysregulation and overexpression of Xq28 CGAs in primary resistance to CTLA4 blockade. Methylation-specific PCR of specific methylation sites of MAGE-A2, MAGE-A3, and MAGE-A12 revealed decreased methylation at these sites in nonresponding (vs. responding) tumors.

As described in detail below, 7 of the top 10 genes overexpressed in primary resistant tumors were cancer-germline antigens (CGA's), ranging from 60-180 fold enrichment. All 7 CGA's clustered tightly together within a narrow 75 kb region of chromosome Xq28. This pattern was clinically validated in Cohort 2 where this specific CGA cluster was similarly enriched in primary resistant tumors. Importantly, this pattern was biologically validated by finding that genes associated with clinical resistance from Cohort 1 significantly overlapped with genes associated with Xq28-CGA cluster expression in The Cancer Genome Atlas (TCGA) melanoma samples, further supporting the association of Xq28 expression with clinical resistance. As described in detail below, TCGA melanoma samples with high expression of the Xq28 CGA locus exhibited profound global hypomethylation, implicating epigenomic dysregulation and overexpression of Xq28 cancer testis antigen (CTA) in primary resistance to CTLA4 blockade.

Moreover, as described in detail below, in patients with clinical benefit, microRNA-211 was enriched over 700-fold, with statistically significant overlap observed between genes associated with clinical response and those associated with miR-211 in TCGA. The Xq28 associated CGA and miR-211 expression signatures were unique to CTLA4 blockade and did not predict outcome to anti-PD1 therapy. Expression levels of Xq28 associated CGAs and miR-211 predicted clinical outcome with 100% sensitivity and 40% specificity, outperforming previously identified correlates of benefit (ROC curve AUC=0.85). Expression of a coordinately transcribed cluster of 7 cancer germline antigens on chromosome Xq28 and miR-211 was strongly associated with resistance and response to anti-CTLA4 therapy respectively in metastatic melanoma. Thus, evaluation of transcriptional activity of these genes informs therapeutic preference in this disease.

Antibodies targeting the CTLA4 pathway in advanced melanoma have yielded durable clinical benefit in a minority of patients. Moreover, the combination of CTLA4 blockade with antagonists to another “immune checkpoint,” the programmed death (PD-1) pathway, increases response rates in metastatic melanoma compared with either agent alone, suggesting the potential for combining CTLA4 blockade with other immunotherapeutics. However, prior to the invention described herein, robust determinants of response and resistance to CTLA4 blockade were elusive, hindering efforts to rationally combine it with other therapies and precisely pair it with patients likely to respond.

Several investigators have identified genomic and transcriptional markers, such as the overall number of somatic mutations, number and clonality of tumor-specific “neoantigens,” and expression of immune genes, to correspond with response. However, the extensive overlap of these molecular signatures between responding and nonresponding tumors precludes their use in predicting clinical outcome. In preclinical studies, epigenetic programs have modulated response to anti-CTLA4 therapy, but prior to the invention described herein, they have not been investigated in large clinical cohorts. To date, discovery of robust predictive molecular signatures have been limited by sample size and lack of validation cohorts. As described in detail below, to interrogate and identify non-genomic determinants of clinical outcome to CTLA4 blockade in advanced melanoma, transcriptomic data from two previously reported clinical treatment cohorts as well as transcriptomic and DNA methylation data from The Cancer Genome Atlas (TCGA) was aggregated and analyzed.

Both as monotherapy and in combination with PD-1 blockade, anti-CTLA4 antibodies have induced substantial clinical benefit in melanoma; yet, prior to the invention described herein, robust molecular signatures of clinical outcome were elusive. Moreover, insight into mechanisms of primary clinical resistance was lacking. Knowledge of both is critical given the increasing evaluation of CTLA4 blockade in hematologic malignancies and the need to both rationally design combinatorial strategies as well as identify new immunotherapeutic targets. Here, the importance of in situ transcriptomic analyses to uncover immunotherapeutically relevant biology was revealed.

In particular, as described in detail below, a critical genomic locus on Xq28 that harbors a coordinately regulated cluster of CT antigens was identified. The striking enrichment of these genes with primary resistance affirms their status as a recent therapeutic target; the encoded proteins have been implicated in the ubiquitination of key tumor suppressors—notably TP53 and AMPK—that contributed to oncogenesis. In fact, these CT antigens specifically cluster in an inverted repeat DNA structure on Xq28, in which they are expressed coordinately and independently from CT antigens outside of this cluster. Thus, the finding that all of these genes appear as the most upregulated genes in resistant tumors reinforces their relevance as a genomic unit to clinical outcome to CTLA4 blockade.

As described in detail below, a co-enrichment of immunosuppressive pathways was identified along with these CT antigens, including the PSG genes as well as the GABA A receptor, which was recently implicated in attenuating T cell priming—a process also governed by the CTLA4 pathway. Associated immunosuppression may explain the long history of failed immunotherapeutic approaches targeting CT antigens such as MAGEA3 and MAGEA6 that lie within the Xq28 locus. The finding of multiple genes involved in epithelial-to-mesenchymal transition (EMT) is consistent with preclinical data suggesting EMT as an immunoevasive pathway employed by melanomas. Moreover, using TCGA data, global hypomethylation patterns were identified that strongly associated with high expression of CTA's from the Xq28 locus, implicating epigenomic mechanisms of resistance to CTLA4 blockade.

In responding tumors, the analysis revealed enrichment of the melanoma-suppressive miR-211 and a diversity of immune effectors, including T cells, B cells, macrophages, and eosinophils. miR-211 has been shown to inhibit TGF-beta signaling members (which were upregulated in resistant tumors), suppress the EMT phenotype, and mitigate the invasive phenotype. An increased number of genes mapping to both T cell and B cell receptors were identified, implicating an active adaptive immune response that appears to be diverse in its antigen recognition.

Although statistical stringency was relaxed because of the small cohort, the converging results from alternative cohorts bolster the results presented herein. Given that CTLA4 blockade may impact immune priming as opposed to the effector arm (influenced by the PD1 pathway), the results presented herein shed light into the mechanisms governing response/resistance to therapeutic manipulation of immune priming. The results presented herein indicate that response/resistance mechanisms to immune priming differ substantially from those relevant to clinical manipulation of effector immunity. As immunotherapeutic combinations are increasingly evaluated, understanding these mechanisms are important for precisely pairing patients with the appropriate combinations to avoid toxicity and ensure efficacy. The gene signatures described herein are potential therapeutic targets to sensitize to or combine with CTLA4 blockade.

Moreover, precisely pairing patients with cancer to the appropriate immunotherapy would reduce toxicity and costs as well as accelerate drug development. While ipilimumab as a single agent can induce durable tumor remissions in metastatic melanoma, only about 15-20% of patients with melanoma will benefit. Thus the majority of patients with melanoma are already resistant to ipilimumab. The results presented herein not only suggest a combination of immunotherapies that would raise this response rate, but they identify a signature to select those patients that would benefit from the combination (e.g. CTLA4 blockade+HMGB1 receptor agonist; or CTLA4 blockade+Xq28-CGA antagonist) over monotherapy. For example, a patient with high expression of the CGA gene would be assigned to the CTLA4 blockade+HMGB1 receptor agonist combination (or CTLA4 blockade+Xq28-CGA antagonist combination), whereas a patient with low expression of the CGA gene would be assigned to anti-CTLA4 monotherapy.

### Melanoma

Cancer starts when cells in the body begin to grow out of control. Cells in nearly any part of the body can become cancer, and can then spread to other areas of the body. Melanoma is a cancer that usually starts in a certain type of skin cell, i.e., melanocytes. Melanocytes make a brown pigment called melanin, which gives the skin its tan or brown color. Melanin protects the deeper layers of the skin from some of the harmful effects of the sun. For most people, when skin is exposed to the sun, melanocytes make more melanin, causing the skin to tan or darken.

Other names for “melanoma” include malignant melanoma and cutaneous melanoma. Most melanoma cells still make melanin, so melanoma tumors are usually brown or black. However, some melanomas do not make melanin and can appear pink, tan, or even white. Melanomas can develop anywhere on the skin, but they are more likely to start on the trunk (chest and back) in men and on the legs in women. The neck and face are other common sites. Having darkly pigmented skin lowers the risk of melanoma at these more common sites, but anyone can get melanoma on the palms of the hands, soles of the feet, and under the nails. Melanomas can also form in other parts of the body such as the eyes, mouth, genitals, and anal area, but these are much less common than melanoma of the skin. Melanoma is much less common than basal cell and squamous cell skin cancers. However, melanoma is more dangerous because it is much more likely to spread to other parts of the body if not caught early.

The primary cause of melanoma is ultraviolet light (UV) exposure in those with low levels of skin pigment. The UV light may be from either the sun or from other sources, such as tanning devices. About 25% develop from moles. Those with many moles, a history of affected family members, and who have poor immune function are at greater risk. A number of rare genetic defects such as xeroderma pigmentosum also increase risk. Avoiding UV light and the use of sunscreen may prevent melanoma.

Melanoma may spread to other sites in the body by metastais. Metastatic melanoma may cause nonspecific paraneoplastic symptoms, including loss of appetite, nausea, vomiting and fatigue. Metastasis of early melanoma is possible, but relatively rare: less than a fifth of melanomas diagnosed early become metastatic. Brain metastases are particularly common in patients with metastatic melanoma. Melanoma may also spread to the liver, bones, abdomen or distant lymph nodes.

### Melanoma Diagnosis

Visual inspection is the most common diagnostic technique. Moles that are irregular in color or shape are typically treated as candidates. To detect melanomas (and increase survival rates), it is recommended to regularly examine moles for changes (shape, size, color, itching or bleeding) and to consult a qualified physician when a candidate appears.

Early signs of melanoma are changes to the shape or color of existing moles or, in the case of nodular melanoma, the appearance of a new lump anywhere on the skin. At later stages, the mole may itch, ulcerate or bleed. Early signs of melanoma are summarized by the mnemonic “ABCDE”:
Asymmetry
Borders (irregular with edges and corners)
Color (variegated)
Diameter (greater than 6 mm (0.24 in), about the size of a pencil eraser)
Evolving over time

These classifications do not, however, apply to the most dangerous form of melanoma, nodular melanoma, which has its own classifications:
Elevated above the skin surface
Firm to the touch
Growing

Following a visual examination and a dermatoscopic exam, or in vivo diagnostic tools such as a confocal microscope, the doctor may biopsy the suspicious mole. A skin biopsy performed under local anesthesia is often required to assist in making or confirming the diagnosis and in defining severity. Elliptical excisional biopsies may remove the tumor, followed by histological analysis and Breslow scoring. Punch biopsies are contraindicated in suspected melanomas, for fear of seeding tumor cells and hastening the spread of malignant cells.

Lactate dehydrogenase (LDH) tests are often used to screen for metastases, although many patients with metastases (even end-stage) have a normal LDH; extraordinarily high LDH often indicates metastatic spread of the disease to the liver.

It is common for patients diagnosed with melanoma to have chest X-rays and an LDH test, and in some cases CT, MRI, PET and/or PET/CT scans. Although controversial, sentinel lymph node biopsies and examination of the lymph nodes are also performed in patients to assess spread to the lymph nodes.

A diagnosis of melanoma is supported by the presence of the S-100 protein marker. Additionally, HMB-45 is a monoclonal antibody that reacts against an antigen present in melanocytic tumors such as melanomas. It is used in anatomic pathology as a marker for such tumors. The antibody was generated to an extract of melanoma. It reacts positively against melanocytic tumors, but not other tumors, thus demonstrating specificity and sensitivity.

The following are melanoma stages with 5 year survival rates. Stage 0: melanoma in situ (99.9% survival); Stage I/II: invasive melanoma (89-95% survival); Stage II: high risk melanoma (45-79% survival); Stage III: regional metastasis (24-70% survival); Stage IV: distant metastasis (7-19% survival).

Recent evidence suggests that the prognosis of melanoma patients with regional metastases is influenced by tumor stroma immunobiology (Akbani et al., 2015 Cell (161), 1681-1696, incorporated herein by reference).

### Melanoma Treatment

Treatment is typically removal by surgery. In those with slightly larger cancers, nearby lymph nodes may be tested for spread. Most people are cured after tumor excision if spread has not occurred. Excisional biopsies may remove the tumor, but further surgery is often necessary to reduce the risk of recurrence. Complete surgical excision with adequate surgical margins and assessment for the presence of detectable metastatic disease along with short- and long-term followup is standard. Often this is done by a wide local excision (WLE) with 1 to 2 cm margins.

For those in whom melanoma has spread, immunotherapy, biologic therapy, radiation therapy, or chemotherapy may improve survival. With treatment, the five-year survival rates in the United States is 98% among those with localized disease and 17% among those in whom spread has occurred. The likelihood that it will come back or spread depends on the melanoma thickness, how fast the cells are dividing, and whether or not the overlying skin has broken down.

Various chemotherapy agents, including temozolomide, dacarbazine (also termed DTIC), immunotherapy (with interleukin-2 (IL-2) or interferon (IFN)), as well as local perfusion, are used for treatment of melanoma. The overall success in metastatic melanoma is quite limited. Therapies for metastatic melanoma include biologic immunotherapy agents ipilimumab, pembrolizumab, and nivolumab; BRAF inhibitors, such as vemurafenib and dabrafenib; and a MEK inhibitor, trametinib.

Radiation therapy is often used after surgical resection for patients with locally or regionally advanced melanoma or for patients with unresectable distant metastases. Kilovoltage x-ray beams are often used for these treatments and have the property of the maximum radiation dose occurring close to the skin surface.

### CTLA-4-Blockade

CTLA4 or CTLA-4 (cytotoxic T-lymphocyte-associated protein 4), also known as CD152 (cluster of differentiation 152), is a protein receptor that, functioning as an immune checkpoint, downregulates immune responses. CTLA4 is constitutively expressed in regulatory T cells (Tregs), but only upregulated in conventional T cells after activation. CTLA4 acts as an “off” switch when bound to CD80 or CD86 on the surface of antigen-presenting cells. Recent reports suggest that blocking CTLA4 (using antagonistic antibodies against CTLA such as ipilimumab (FDA approved for melanoma in 2011)) results in therapeutic benefit. CTLA4 blockade inhibits immune system tolerance to tumors and provides a useful immunotherapy strategy for patients with cancer. See, Grosso J. and Jure-Kunkel M. 2013, Cancer Immun., 13: 5, incorporated herein by reference.

Ipilimumab, a fully human monoclonal antibody specific to CTLA-4, improves overall survival in metastatic melanoma patients (Ji et al., 2012 Cancer Immunol Immunother, 61: 1019-1031, incorporated herein by reference). Indeed, monoclonal antibodies directed against CTLA4, such as ipilimumab, yield considerable clinical benefit for patients with metastatic melanoma by inhibiting checkpoint activity; however, prior to the invention described herein, clinical predictors of response to these therapies were incompletely characterized (Van Allen, et al., 2015 Science, 350(6257): 207-211, incorporated herein by reference). See also, Snyder et al., 2014 The New England Journal of Medicine, 373(20): 1984, incorporated herein by reference.

### World Health Organization Criteria

The WHO Criteria for evaluating the effectiveness of anti-cancer agents on tumor shrinkage, developed in the 1970s by the International Union Against Cancer and the World Health Organization, represented the first generally agreed specific criteria for the codification of tumor response evaluation. These criteria were first published in 1981 (Miller et al., 1981 Clin Cancer Res., 47(1): 207-14, incorporated herein by reference). WHO Criteria proposed >50% tumour shrinkage for a Partial Response and >25% tumour increase for Progressive Disease.

### Response Evaluation Criteria in Solid Tumors (RECIST)

RECIST is a set of published rules that define when tumors in cancer patients improve (“respond”), stay the same (“stabilize”), or worsen (“progress”) during treatment (Eisenhauer et al., 2009 European Journal of Cancer, 45: 228-247, incorporated herein by reference). Only patients with measurably disease at baseline should be included in protocols where objective tumor response is the primary endpoint.

The response criteria for evaluation of target lesions are as follows:
Complete Response (CR): Disappearance of all target lesions.
Partial Response (PR): At least a 30% decrease in the sum of the longest diameter (LD) of target lesions, taking as reference the baseline sum LD.
Stable Disease (SD): Neither sufficient shrinkage to qualify for PR nor sufficient increase to qualify for PD, taking as reference the smallest sum LD since the treatment started.
Progressive Disease (PD): At least a 20% increase in the sum of the LD of target lesions, taking as reference the smallest sum LD recorded since the treatment started or the appearance of one or more new lesions.
The response criteria for evaluation of non-target lesions are as follows:
Complete Response (CR): Disappearance of all non-target lesions and normalization of tumor marker level.
Incomplete Response/Stable Disease (SD): Persistence of one or more non-target lesion(s) or/and maintenance of tumor marker level above the normal limits.
Progressive Disease (PD): Appearance of one or more new lesions and/or unequivocal progression of existing non-target lesions.

The response criteria for evaluation of best overall response are as follows. The best overall response is the best response recorded from the start of the treatment until disease progression/recurrence (taking as reference for PD the smallest measurements recorded since the treatment started). In general, the patient's best response assignment will depend on the achievement of both measurement and confirmation criteria.
Patients with a global deterioration of health status requiring discontinuation of treatment without objective evidence of disease progression at that time should be classified as having “symptomatic deterioration”. Every effort should be made to document the objective progression even after discontinuation of treatment.
In some circumstances, it may be difficult to distinguish residual disease from normal tissue. When the evaluation of complete response depends on this determination, it is recommended that the residual lesion be investigated (fine needle aspirate/biopsy) to confirm the complete response status.

### Immune-Related Response Criteria

The immune-related response criteria (irRC) is a set of published rules that define when tumors in cancer patients improve (“respond”), stay the same (“stabilize”), or worsen (“progress”) during treatment, where the compound being evaluated is an immuno-oncology drug. The Immune-Related Response Criteria, first published in 2009 (Wolchok et al., 2009 Clin Cancer Res, 15(23):7412, incorporated herein by reference), arose out of observations that immuno-oncology drugs would fail in clinical trials that measured responses using the WHO or RECIST Criteria, because these criteria could not account for the time gap in many patients between initial treatment and the apparent action of the immune system to reduce the tumor burden. The key driver in the development of the irRC was the observation that, in studies of various cancer therapies derived from the immune system such as cytokines and monoclonal antibodies, the looked-for Complete and Partial Responses as well as Stable Disease only occurred after an increase in tumor burden that the conventional RECIST Criteria would have dubbed “Progressive Disease’. RECIST failed to take account of the delay between dosing and an observed anti-tumour T cell response, so that otherwise ‘successful’ drugs—that is, drugs which ultimately prolonged life—failed in clinical trials.

The irRC are based on the WHO Criteria; however, the measurement of tumor burden and the assessment of immune-related response have been modified as set forth below.

### Measurement of Tumor Burden

In the irRC, tumor burden is measured by combining ‘index’ lesions with new lesions. Ordinarily, tumor burden would be measured with a limited number of ‘index’ lesions (that is, the largest identifiable lesions) at baseline, with new lesions identified at subsequent timepoints counting as ‘Progressive Disease’. In the irRC, by contrast, new lesions are a change in tumor burden. The irRC retained the bidirectional measurement of lesions that had originally been laid down in the WHO Criteria.

### Assessment of Immune-Related Response

In the irRC, an immune-related Complete Response (irCR) is the disappearance of all lesions, measured or unmeasured, and no new lesions; an immune-related Partial Response (irPR) is a 50% drop in tumor burden from baseline as defined by the irRC; and immune-related Progressive Disease (irPD) is a 25% increase in tumor burden from the lowest level recorded. Everything else is considered immune-related Stable Disease (irSD). Even if tumor burden is rising, the immune system is likely to “kick in” some months after first dosing and lead to an eventual decline in tumor burden for many patients. The 25% threshold accounts for this apparent delay.

### The Cancer Genome Atlas (TCGA)

The Cancer Genome Atlas (TCGA) is a project to catalogue genetic mutations responsible for cancer, using genome sequencing and bioinformatics (Cancer Genome Atlas N. Genomic Classification of Cutaneous Melanoma. 2015 Cell, 161(7):1681-96, incorporated herein by reference). TCGA applies high-throughput genome analysis techniques to improve the ability to diagnose, treat, and prevent cancer through a better understanding of the genetic basis of this disease.

The project scheduled 500 patient samples, more than most genomics studies, and used different techniques to analyze the patient samples. Techniques include gene expression profiling, copy number variation profiling, SNP genotyping, genome wide DNA methylation profiling, microRNA profiling, and exon sequencing of at least 1,200 genes. TCGA is sequencing the entire genomes of some tumors, including at least 6,000 candidate genes and microRNA sequences. This targeted sequencing is being performed by all three sequencing centers using hybrid-capture technology. In phase II, TCGA is performing whole exon sequencing on 80% of the cases and whole genome sequencing on 80% of the cases used in the project.

### Gene Expression Profiling

In general, methods of gene expression profiling can be divided into two large groups: methods based on hybridization analysis of polynucleotides, and methods based on sequencing of polynucleotides. Methods known in the art for the quantification of mRNA expression in a sample include northern blotting and in situ hybridization, RNAse protection assays, RNA-seq, and reverse transcription polymerase chain reaction (RT-PCR). Alternatively, antibodies are employed that recognize specific duplexes, including DNA duplexes, RNA duplexes, and DNA-RNA hybrid duplexes or DNA-protein duplexes. Representative methods for sequencing-based gene expression analysis include Serial Analysis of Gene Expression (SAGE), and gene expression analysis by massively parallel signature sequencing (MPSS). For example, RT-PCR is used to compare mRNA levels in different sample populations, in normal and tumor tissues, with or without drug treatment, to characterize patterns of gene expression, to discriminate between closely related mRNAs, and/or to analyze RNA structure.

In some cases, a first step in gene expression profiling by RT-PCR is the reverse transcription of the RNA template into cDNA, followed by amplification in a PCR reaction. For example, extracted RNA is reverse-transcribed using a GeneAmp RNA PCR kit (Perkin Elmer, Calif., USA), following the manufacturer's instructions. The cDNA is then used as template in a subsequent PCR amplification and quantitative analysis using, for example, a TaqMan® (Life Technologies, Inc., Grand Island, N.Y.) assay.

### Microarrays

Differential gene expression can also be identified, or confirmed using a microarray technique. In these methods, polynucleotide sequences of interest (including cDNAs and oligonucleotides) are plated, or arrayed, on a microchip substrate. The arrayed sequences are then hybridized with specific DNA probes from cells or tissues of interest. Just as in the RT-PCR method, the source of mRNA typically is total RNA isolated from human tumors or tumor cell lines and corresponding normal tissues or cell lines. Thus, RNA is isolated from a variety of primary tumors or tumor cell lines. If the source of mRNA is a primary tumor, mRNA is extracted from frozen or archived tissue samples.

In the microarray technique, PCR-amplified inserts of cDNA clones are applied to a substrate in a dense array. The microarrayed genes, immobilized on the microchip, are suitable for hybridization under stringent conditions.

In some cases, fluorescently labeled cDNA probes are generated through incorporation of fluorescent nucleotides by reverse transcription of RNA extracted from tissues of interest (e.g., melanoma tissue). Labeled cDNA probes applied to the chip hybridize with specificity to loci of DNA on the array. After washing to remove non-specifically bound probes, the chip is scanned by confocal laser microscopy or by another detection method, such as a charge-coupled device (CCD) camera. Quantification of hybridization of each arrayed element allows for assessment of corresponding mRNA abundance.

In some configurations, dual color fluorescence is used. With dual color fluorescence, separately labeled cDNA probes generated from two sources of RNA are hybridized pairwise to the array. The relative abundance of the transcripts from the two sources corresponding to each specified gene is thus determined simultaneously. In various configurations, the miniaturized scale of the hybridization can afford a convenient and rapid evaluation of the expression pattern for large numbers of genes. In various configurations, such methods can have sensitivity required to detect rare transcripts, which are expressed at fewer than 1000, fewer than 100, or fewer than 10 copies per cell. In various configurations, such methods can detect at least approximately two-fold differences in expression levels (Schena et al., Proc. Natl. Acad. Sci. USA 93(2): 106-149 (1996)). In various configurations, microarray analysis is performed by commercially available equipment, following manufacturer's protocols, such as by using the Affymetrix GenChip technology, or Incyte's microarray technology.

### RNA-seq

RNA sequencing (RNA-seq), also called whole transcriptome shotgun sequencing (WTSS), uses next-generation sequencing (NGS) to reveal the presence and quantity of RNA in a biological sample at a given moment in time.

RNA-Seq is used to analyze the continually changing cellular transcriptome. See, e.g., Wang et al., 2009 Nat Rev Genet, 10(1): 57-63, incorporated herein by reference. Specifically, RNA-Seq facilitates the ability to look at alternative gene spliced transcripts, post-transcriptional modifications, gene fusion, mutations/SNPs and changes in gene expression. In addition to mRNA transcripts, RNA-Seq can look at different populations of RNA to include total RNA, small RNA, such as miRNA, tRNA, and ribosomal profiling. RNA-Seq can also be used to determine exon/intron boundaries and verify or amend previously annotated 5′ and 3′ gene boundaries.

Prior to RNA-Seq, gene expression studies were done with hybridization-based microarrays. Issues with microarrays include cross-hybridization artifacts, poor quantification of lowly and highly expressed genes, and needing to know the sequence of interest. Because of these technical issues, transcriptomics transitioned to sequencing-based methods. These progressed from Sanger sequencing of Expressed Sequence Tag libraries, to chemical tag-based methods (e.g., serial analysis of gene expression), and finally to the current technology, NGS of cDNA (notably RNA-Seq).

### Gene Signature

As described here, a gene signature was defined herein, which distinguishes CTLA-4 response in melanoma patients. Also described herein is a gene signature which distinguishes response to a combination of CTLA-4 blockade and a TLR (or autophagy) agonist. Exemplary distinguishing genes are provided below.

An exemplary human MAGEA2 amino acid sequence is set forth below (SEQ ID NO: 1; GenBank Accession No: NP_001269434, Version 1, incorporated herein by reference):

| | |
|---|---|
| 1 | mpleqrsqhc kpeeglearg ealglvgaqa pateeqqtas ssstlvevtl gevpaadsps |
| 61 | pphspqgass fsttinytlw rqsdegssnq eeegprmfpd lesefqaais rkmvelvhfl |
| 121 | llkyrarepv tkaemlesvl rncqdffpvi fskaseylql vfgievvevv pishlyilvt |
| 181 | clglsydgll gdnqvmpktg lliivlaiia iegdcapeek iweelsmlev fegredsvfa |
| 241 | hprkllmqdl vqenyleyrq vpgsdpacye flwgpralie tsyvkvlhht lkiggephis |
| 301 | ypplheralr egee |

An exemplary human MAGEA2 nucleic acid sequence is set forth below (SEQ ID NO: 2; GenBank Accession No: NM_001282505, Version 1, incorporated herein by reference):

| | |
|---|---|
| 1 | ttgcgcattg gaggtcagag gacagcgaga ttctcgccct gagcaacggc ctgacgtcgg |
| 61 | cggagggaag caggcgcagg ctccgtgagg aggcaaggcc tgtgggtctt cattgcccag |
| 121 | ctcctgcccg cactcctgcc tgctgccctg accagagtca tcatgcctct tgagcagagg |
| 181 | agtcagcact gcaagcctga agaaggcctt gaggcccgag gagaggccct gggcctggtg |
| 241 | ggtgcgcagg ctcctgctac tgaggagcag cagaccgctt cttcctcttc tactctagtg |
| 301 | gaagttaccc tgggggaggt gcctgctgcc gactcaccga gtcctcccca cagtcctcag |
| 361 | ggagcctcca gcttctcgac taccatcaac tacactcttt ggagacaatc cgatgagggc |
| 421 | tccagcaacc aagaagagga ggggccaaga atgtttcccg acctggagtc cgagttccaa |
| 481 | gcagcaatca gtaggaagat ggttgagttg gttcattttc tgctcctcaa gtatcgagcc |
| 541 | agggagccgg tcacaaaggc agaaatgctg gagagtgtcc tcagaaattg ccaggacttc |
| 601 | tttcccgtga tcttcagcaa agcctccgag tacttgcagc tggtctttgg catcgaggtg |
| 661 | gtggaagtgg tccccatcag ccacttgtac atccttgtca cctgcctggg cctctcctac |
| 721 | gatggcctgc tgggcgacaa tcaggtcatg cccaagacag gcctcctgat aatcgtcctg |
| 781 | gccataatcg caatagaggg cgactgtgcc cctgaggaga aaatctggga ggagctgagt |
| 841 | atgttggagg tgtttgaggg gagggaggac agtgtcttcg cacatcccag gaagctgctc |
| 901 | atgcaagatc tggtgcagga aaactacctg gagtaccggc aggtgcccgg cagtgatcct |
| 961 | gcatgctacg agttcctgtg gggtccaagg gccctcattg aaaccagcta tgtgaaagtc |
| 1021 | ctgcaccata cactaaagat cggtggagaa cctcacattt cctacccacc cctgcatgaa |
| 1081 | cgggctttga gagagggaga agagtgagtc tcagcacatg ttgcagccag ggccagtggg |
| 1141 | agggggtctg ggccagtgca ccttccaggg ccccatccat tagcttccac tgcctcgtgt |
| 1201 | gatatgaggc ccattcctgc ctctttgaag agagcagtca gcattcttag cagtgagttt |
| 1261 | ctgttctgtt ggatgacttt gagatttatc tttgtttcct gttggaattg ttcaaatgtt |
| 1321 | ccttttaaca aatggttgga tgaacttcag catccaagtt tatgaatgac agtagtcaca |
| 1381 | catagtgctg tttatatagt ttaggggtaa gagtcctgtt ttttattcag attgggaaat |
| 1441 | ccattccatt ttgtgagttg tcacataata acagcagtgg aatatgtatt tgcctatatt |
| 1501 | gtgaacgaat tagcagtaaa atacatgata caaggaactc aaaagatagt taattcttgc |
| 1561 | cttatacctc agtctattat gtaaaattaa aaatatgtgt atgtttttgc ttctttgaga |
| 1621 | atgcaaaaga aattaaatct gaataaataa ttcttcctgt tcaaaaaaaa aaaaaaaaaa |

An exemplary human MAGEA3 amino acid sequence is set forth below (SEQ ID NO: 3; GenBank Accession No: CAG46566.1, Version 1, incorporated herein by reference):

| | |
|---|---|
| 1 | mpleqrsqhc kpeeglearg ealglvgaqa pateeqeaas ssstivevtl gevpaaespd |
| 61 | ppqspqgass lpttmnyplw sqsyedssnq eeegpstfpd lesefqaals rkvaelvhfl |
| 121 | llkyrarepv tkaemlgsvv gnwqyffpvi fskassslql vfgielmevd pighlyifat |
| 181 | clglsydgll gdnqimpkag lliivlaiia regdcapeek iweelsvlev fegredsilg |
| 241 | dpkklltqhf vqenyleyrq vpgsdpacye flwgpralve tsyvkvlhhm vkisggphis |
| 301 | ypplhewvlr egee |

An exemplary human MAGEA3 nucleic acid sequence is set forth below (SEQ ID NO: 4; GenBank Accession No: NM_005362.3, Version 3, incorporated herein by reference):

| | |
|---|---|
| 1 | gagattctcg ccctgagcaa cgagcgacgg cctgacgtcg gcggagggaa gccggcccag |
| 61 | gctcggtgag gaggcaaggt tctgagggga caggctgacc tggaggacca gaggcccccg |
| 121 | gaggagcact gaaggagaag atctgccagt gggtctccat tgcccagctc ctgcccacac |
| 181 | tcccgcctgt tgccctgacc agagtcatca tgcctcttga gcagaggagt cagcactgca |
| 241 | agcctgaaga aggccttgag gcccgaggag aggccctggg cctggtgggt gcgcaggctc |
| 301 | ctgctactga ggagcaggag gctgcctcct cctcttctac tctagttgaa gtcaccctgg |
| 361 | gggaggtgcc tgctgccgag tcaccagatc ctccccagag tcctcaggga gcctccagcc |
| 421 | tccccactac catgaactac cctctctgga gccaatccta tgaggactcc agcaaccaag |
| 481 | aagaggaggg gccaagcacc ttccctgacc tggagtccga gttccaagca gcactcagta |
| 541 | ggaaggtggc cgagttggtt cattttctgc tcctcaagta tcgagccagg gagccggtca |
| 601 | caaaggcaga aatgctgggg agtgtcgtcg gaaattggca gtatttcttt cctgtgatct |
| 661 | tcagcaaagc ttccagttcc ttgcagctgg tctttggcat cgagctgatg gaagtggacc |
| 721 | ccatcggcca cttgtacatc tttgccacct gcctgggcct ctcctacgat ggcctgctgg |
| 781 | gtgacaatca gatcatgccc aaggcaggcc tcctgataat cgtcctggcc ataatcgcaa |
| 841 | gagagggcga ctgtgcccct gaggagaaaa tctgggagga gctgagtgtg ttagaggtgt |
| 901 | ttgaggggag ggaagacagt atcttggggg atcccaagaa gctgctcacc caacatttcg |
| 961 | tgcaggaaaa ctacctggag taccggcagg tccccggcag tgatcctgca tgttatgaat |
| 1021 | tcctgtgggg tccaagggcc ctcgttgaaa ccagctatgt gaaagtcctg caccatatgg |
| 1081 | taaagatcag tggaggacct cacatttcct acccacccct gcatgagtgg gttttgagag |
| 1141 | agggggaaga gtgagtctga gcacgagttg cagccagggc cagtgggagg gggtctgggc |
| 1201 | cagtgcacct tccggggccg catcccttag tttccactgc ctcctgtgac gtgaggccca |
| 1261 | ttcttcactc tttgaagcga gcagtcagca ttcttagtag tgggtttctg ttctgttgga |
| 1321 | tgactttgag attattcttt gtttcctgtt ggagttgttc aaatgttcct tttaacggat |
| 1381 | ggttgaatga gcgtcagcat ccaggtttat gaatgacagt agtcacacat agtgctgttt |
| 1441 | atatagttta ggagtaagag tcttgttttt tactcaaatt gggaaatcca ttccattttg |
| 1501 | tgaattgtga cataataata gcagtggtaa aagtatttgc ttaaaattgt gagcgaatta |
| 1561 | gcaataacat acatgagata actcaagaaa tcaaaagata gttgattctt gccttgtacc |
| 1621 | tcaatctatt ctgtaaaatt aaacaaatat gcaaaccagg atttccttga cttctttgag |
| 1681 | aatgcaagcg aaattaaatc tgaataaata attcttcctc ttcaaaaaaa aaaaaaaaaa |
| 1741 | aaaaaaaaaa aaa |

An exemplary human MAGEA6 amino acid sequence is set forth below (SEQ ID NO: 5; GenBank Accession No: CAG46567.1, Version 1, incorporated herein by reference):

| | |
|---|---|
| 1 | mpleqrsqhc kpeeglearg ealglvgaqa pateeqeaas ssstlvevtl gevpaaespd |
| 61 | ppgspqgass lpttmnyplw sqsyedssnq eeegpstfpd lesefqaals rkvaklvhfl |
| 121 | llkyrarepv tkaemlgsvv gnwqyffpvi fskasdslql vfgielmevd pighvyifat |
| 181 | clglsydgll gdnqimpktg fliiilaiia kegdcapeek iweelsvlev fegredsifg |
| 241 | dpkklltqyf vqenyleyrq vpgsdpacye flwgpralie tsyvkvlhhm vkisggpris |
| 301 | ypllhewafr egee |

An exemplary human MAGEA6 nucleic acid sequence is set forth below (SEQ ID NO: 6; GenBank Accession No: NM_005362.3, Version 3, incorporated herein by reference):

| | |
|---|---|
| 1 | gagattctcg ccctgagcaa cgagcgacgg cctgacgtcg gcggagggaa gccggcccag |
| 61 | gctcggtgag gaggcaaggt tctgagggga caggctgacc tggaggacca gaggcccccg |
| 121 | gaggagcact gaaggagaag atctgccagt gggtctccat tgcccagctc ctgcccacac |
| 181 | tcccgcctgt tgccctgacc agagtcatca tgcctcttga gcagaggagt cagcactgca |
| 241 | agcctgaaga aggccttgag gcccgaggag aggccctggg cctggtgggt gcgcaggctc |
| 301 | ctgctactga ggagcaggag gctgcctcct cctcttctac tctagttgaa gtcaccctgg |
| 361 | gggaggtgcc tgctgccgag tcaccagatc ctccccagag tcctcaggga gcctccagcc |
| 421 | tccccactac catgaactac cctctctgga gccaatccta tgaggactcc agcaaccaag |
| 481 | aagaggaggg gccaagcacc ttccctgacc tggagtccga gttccaagca gcactcagta |
| 541 | ggaaggtggc cgagttggtt cattttctgc tcctcaagta tcgagccagg gagccggtca |
| 601 | caaaggcaga aatgctgggg agtgtcgtcg gaaattggca gtatttcttt cctgtgatct |
| 661 | tcagcaaagc ttccagttcc ttgcagctgg tctttggcat cgagctgatg gaagtggacc |
| 721 | ccatcggcca cttgtacatc tttgccacct gcctgggcct ctcctacgat ggcctgctgg |
| 781 | gtgacaatca gatcatgccc aaggcaggcc tcctgataat cgtcctggcc ataatcgcaa |
| 841 | gagagggcga ctgtgcccct gaggagaaaa tctgggagga gctgagtgtg ttagaggtgt |
| 901 | ttgaggggag ggaagacagt atcttggggg atcccaagaa gctgctcacc caacatttcg |
| 961 | tgcaggaaaa ctacctggag taccggcagg tccccggcag tgatcctgca tgttatgaat |
| 1021 | tcctgtgggg tccaagggcc ctcgttgaaa ccagctatgt gaaagtcctg caccatatgg |
| 1081 | taaagatcag tggaggacct cacatttcct acccacccct gcatgagtgg gttttgagag |
| 1141 | agggggaaga gtgagtctga gcacgagttg cagccagggc cagtgggagg gggtctgggc |
| 1201 | cagtgcacct tccggggccg catcccttag tttccactgc ctcctgtgac gtgaggccca |
| 1261 | ttcttcactc tttgaagcga gcagtcagca ttcttagtag tgggtttctg ttctgttgga |
| 1321 | tgactttgag attattcttt gtttcctgtt ggagttgttc aaatgttcct tttaacggat |
| 1381 | ggttgaatga gcgtcagcat ccaggtttat gaatgacagt agtcacacat agtgctgttt |
| 1441 | atatagttta ggagtaagag tcttgttttt tactcaaatt gggaaatcca ttccattttg |
| 1501 | tgaattgtga cataataata gcagtggtaa aagtatttgc ttaaaattgt gagcgaatta |
| 1561 | gcaataacat acatgagata actcaagaaa tcaaaagata gttgattctt gccttgtacc |
| 1621 | tcaatctatt ctgtaaaatt aaacaaatat gcaaaccagg atttccttga cttctttgag |
| 1681 | aatgcaagcg aaattaaatc tgaataaata attcttcctc ttcaaaaaaa aaaaaaaaaa |
| 1741 | aaaaaaaaaa aaa |

An exemplary human MAGEA12 amino acid sequence is set forth below (SEQ ID NO: 7; GenBank Accession No: EAW99432.1, Version 1, incorporated herein by reference):

| | |
|---|---|
| 1 | mpleqrsqhc kpeegleaqg ealglvgaqa pateeqetas ssstlvevtl revpaaesps |
| 61 | pphspqgast lpttinytlw sqsdegssne eqegpstfpd letsfqvals rkmaelvhfl |
| 121 | llkyrarepf tkaemlgsvi rnfqdffpvi fskaseylql vfgievvevv righlyilvt |
| 181 | clglsydgll gdnqivpktg lliivlaiia kegdcapeek iweelsvlea sdgredsvfa |
| 241 | hprklltqdl vqenyleyrq vpgsdpacye flwgpralve tsyvkvlhhl lkisggphis |
| 301 | ypplhewafr egee |

An exemplary human MAGEA12 nucleic acid sequence is set forth below (SEQ ID NO: 8; GenBank Accession No: NM_001166386.3, Version 3, incorporated herein by reference):

| | |
|---|---|
| 1 | aagatgccga gggaggactg aggcgggcct caccccagac agagggcccc caataatcca |
| 61 | gcgctgcctc tgctgccggg cctggaccac cctgcagggg aagacttctc aggctgagtc |
| 121 | gccaccacct caccccgcca ccccccgccg ctttaaccgc agggaactct ggtatctcag |
| 181 | ggagttgagg accttttctt cagagggtga ctcaggtcaa cacaggggcc cccatgtagt |
| 241 | cgacagacac agtggtccta agatctacca agcatccagg ttctgaggag acaggccccg |
| 301 | gagcagcact agctcctgcc cacactccta cctgctgccc tgaccagagt catcatgcca |
| 361 | cttgagcaga ggagtcagca ctgcaagcct gaggaaggcc ttgaggccca aggagaggcc |
| 421 | ctgggcttgg tgggtgcgca ggctcctgct actgaggagc aggagactgc ctcctcctcc |
| 481 | tctactctag tggaagtcac cctgcgggag gtgcctgctg ccgagtcacc aagtcctccc |
| 541 | cacagtcctc agggagcctc caccctcccc actaccatca actatactct ctggagtcaa |
| 601 | tccgatgagg gctccagcaa cgaagaacag gaagggccaa gcacctttcc tgacctggag |
| 661 | acgagcttcc aagtagcact cagtaggaag atggctgagt tggttcattt tctgctcctc |
| 721 | aagtatcgag ccagggagcc attcacaaag gcagaaatgc tggggagtgt catcagaaat |
| 781 | ttccaggact tctttcctgt gatcttcagc aaagcctccg agtacttgca gctggtcttt |
| 841 | ggcatcgagg tggtggaagt ggtccgcatc ggccacttgt acatccttgt cacctgcctg |
| 901 | ggcctctcct acgatggcct gctgggcgac aatcagatcg tgcccaagac aggcctcctg |
| 961 | ataatcgtcc tggccataat cgcaaaagag ggcgactgtg cccctgagga gaaaatctgg |
| 1021 | gaggagctga gtgtgttgga ggcatctgat gggagggagg acagtgtctt tgcgcatccc |
| 1081 | aggaagctgc tcacccaaga tttggtgcag gaaaactacc tggagtaccg gcaggtcccc |
| 1141 | ggcagtgatc ctgcatgcta cgagttcctg tggggtccaa gggccctcgt tgaaaccagc |
| 1201 | tatgtgaaag tcctgcacca tttgctaaag atcagtggag gacctcacat ttcctaccca |
| 1261 | cccctgcatg aatgggcttt tagagagggg gaagagtgag tctgagcacg agttgcagcc |
| 1321 | agggccagtg ggagggggtc tgggccagtg caccttccaa ggccccatcc attagtttcc |
| 1381 | actgcctcgt gtgacatgag gcccattctt cactctttga agagagcagt cagtattgtt |
| 1441 | agtagtgagt ttctgttcta ttggatgact ttgagattta tctttgtttc ctgttggaat |
| 1501 | tgttcaaatg ttccttttaa cggatggttg aatgaacttc agcatccaag tttatgaatg |
| 1561 | acagtagtca cacatagtgc tgtttatata gtttaggagt aagagtgttg ttttttattc |
| 1621 | agattgggaa atccattcca ttttgtgaat tgtgacaaat aacagcagtg gaaaaagtat |
| 1681 | gtgcttagaa ttgtgaaaga attagcagta aaatacatga gataaagacc tcaagaagtt |
| 1741 | aaaagatact taattcttgc cttatacctc actctattct gtaaatttga aaaaaaagca |
| 1801 | tggatacctg gatatccttg gcttctttga gaatttaaga gaaattaaat ctgaataaat |
| 1861 | aa |

An exemplary human CSAG1 amino acid sequence is set forth below (SEQ ID NO: 9; GenBank Accession No: AAH59947.1, Version 1, incorporated herein by reference):

| | |
|---|---|
| 1 | msattacwpa ftvlgeargd qvdwsrlyrd tglvkmsrkp rasspfsnnh pstpkrfprq |
| 61 | prrekgpvke vpgtkgsp |

An exemplary human CSAG1 nucleic acid sequence is set forth below (SEQ ID NO: 10; GenBank Accession No: BC059947.1, Version 1, incorporated herein by reference):

| | |
|---|---|
| 1 | ctggattctt cctggatggg gatccagatg gaggtggagg gttgatttgg gaagcagagc |
| 61 | acagcagccc aaatttgctt gtaatgtcgg cgactacagc ctgctggcct gccttcactg |
| 121 | tcctggggga agctcgggga gaccaggtgg actggagtag actgtacaga gacactggtc |
| 181 | tggtgaagat gtccaggaaa ccacgagcct ccagcccatt ttccaacaac cacccatcaa |
| 241 | caccaaagag gttcccaaga caacccagaa gggaaaaggg acccgtcaag gaagttccag |
| 301 | gaacaaaagg ctctccctaa aagaccaccg cttcaaaaaa acctgaggaa tggagtgggc |
| 361 | caacactatc cagccactct gaccagccga acgaggaact caatcaaaat gcgccatagc |
| 421 | aggaccacaa gggcaaggag accaccgcct tctccagtgc ttccttgggc agccagtaat |
| 481 | tcccaggcaa ggccagagac ttcaagtcta tctgaaaagt ctccagaagt ctaaccccag |
| 541 | ataaatagcc aacagggtgt agagtacgtt ttacacccca aagggtatgc cccatggtga |
| 601 | tggaaataaa atgaacatgt tgtaaaaaaa aaaaaaaaaa aaa |

An exemplary human CSAG2 amino acid sequence is set forth below (SEQ ID NO: 11; GenBank Accession No: EAW99427.1, Version 1, incorporated herein by reference):

| | |
|---|---|
| 1 | mwmgliqlve gvkrkdqgfl ekefyhktni kmrceflacw paftvlgeaw rdqvdwsrll |
| 61 | rdaglvkmsr kprassplsn nhpptpkrrg sgrhpinpgp ealskfprqp grekgpikev |
| 121 | pgtkgsp |

An exemplary human CSAG2 nucleic acid sequence is set forth below (SEQ ID NO: 12; GenBank Accession No: AJ844639.1, Version 1, incorporated herein by reference):

| | |
|---|---|
| 1 | agttccagga acaaaaggct ctccctaaaa gaccgccgct tcaaaaaaac ctgaggaatg |
| 61 | gagtgggcca acactatcca gccactctga ccagccgaac gaggaactca atcaaaatga |
| 121 | gccatagcgg gaccacaagg gcaaggagac caccaccttc tccagtctct cttcggacag |
| 181 | ccagtaattc ccgggcaagg ccagagactt caa |

An exemplary human CSAG3 amino acid sequence is set forth below (SEQ ID NO: 13; GenBank Accession No: AAI19736.1, Version 1, incorporated herein by reference):

| | |
|---|---|
| 1 | mwmgliqlve gvkrkdqgfl ekefyhktni kmrceflacw paftvlgeaw rdqvdwsrll |
| 61 | rdaglvkmsr kprassplsn nhpptpkrfp rqpgrekgpi kevpgtkgsp |

An exemplary human CSAG3 nucleic acid sequence is set forth below (SEQ ID NO: 14; GenBank Accession No: NM_001129826.2, Version 2, incorporated herein by reference):

| | |
|---|---|
| 1 | gtgcaatggc tagtactatg tgtcaacttg tctaggctat actgctcagc tgtgtggtca |
| 61 | aacagtagtc tagatgttgc tgtgaaggta ttttgtagat gtgatcaaca tttacaatca |
| 121 | gttgatttta agtaaagcag tttaacttcc ataatgtgga tgggcctcat ccaattagtt |
| 181 | gaaggtgtta agagaaaaga ccaaggtttc ctggaaaagg aattctacca caagactaac |
| 241 | ataaaaatgc gctgtgagtt tctagcctgc tggcctgcct tcactgtcct gggggaggct |
| 301 | tggagagacc aggtggactg gagtagactg ttgagagacg ctggtctggt gaagatgtcc |
| 361 | aggaaaccac gagcctccag cccattgtcc aacaaccacc caccaacacc aaagaggcga |
| 421 | ggaagtggaa ggcatcctct caaccctggc ccagaagccc tatcaaagtt cccaagacaa |
| 481 | cccggaaggg aaaagggacc catcaaggaa gttccaggaa caaaaggctc tccctaaaag |
| 541 | accgccgctt caaaaaaacc tgaggaatgg agtgggccaa cactatccag ccactctgac |
| 601 | cagccgaacg aggaactcaa tcaaaatgag ccatagcggg accacaaggg caaggagacc |
| 661 | accaccttct ccagtctctc ttcggacagc cagtaattcc cgggcaaggc cagagacttc |
| 721 | aagtctatct gaaaagtctc cagaggtcta accccagata aatagccaac agggtgtaga |
| 781 | gtacatttta caccccaaag agtgtgcccc atggtgatga aaataaagtg aacatgttgc |
| 841 | aaaatga |

An exemplary human PSG1 amino acid sequence is set forth below (SEQ ID NO: 15; GenBank Accession No: AAH58285.1, Version 1, incorporated herein by reference):

| | |
|---|---|
| 1 | mgtlsappct qrikwkglll tasllnfwnl pttaqvtiea eptkvsegkd vlllvhnlpq |
| 61 | nltgyiwykg qmrdlyhyit syvvdgeiii ygpaysgret aysnaslliq nvtredagsy |
| 121 | tlhiikgddg trgvtgrftf tlhletpkps isssnlnpre tmeaysltcd petpdasylw |
| 181 | wmngqslpmt hslklsetnr tlfllgvtky tagpyeceir npvsasrsdp vtlnllpklp |
| 241 | kpyitinnln prenkdvinf tcepksenyt yiwwlngqsl pvsprvkrpi enrililpsv |
| 301 | trnetgpyqc eirdryggir sdpvtlnvly gpdlpriyps ftyyrsgevl ylscsadsnp |
| 361 | paqyswtine kfqlpgqklf irhittkhsg lyvcsvrnsa tgkessksmt vevsdwtvp |

An exemplary human PSG1 nucleic acid sequence is set forth below (SEQ ID NO: 16; GenBank Accession No: M93704.1, Version 1, incorporated herein by reference):

| | |
|---|---|
| 1 | cgaagctgcc caagccctac atcaccatca acaacttaaa ccccagggag aataaggatg |
| 61 | tcttaaactt cacctgtgaa cctaagagtg agaactacac ctacatttcg tggctaaatg |
| 121 | gtcagagcct cccggtcagt cccagggtaa agcgacccat tgaaaacagg atcctcattc |
| 181 | tacccagtgt cacgagaaat gaaacaggac cctatcaatg tgaaatacgg gaccgatatg |
| 241 | gtggcatccg cagtgaccca gtcaccctga atgtcctct |

An exemplary human PSG2 amino acid sequence is set forth below (SEQ ID NO: 17; GenBank Accession No: AAH22316.1, Version 1, incorporated herein by reference):

| | |
|---|---|
| 1 | mgplsappct ehikwkglll tasllnfwnl pttaqvtiea qppkvsegkd vlllvhnlpq |
| 61 | nltgyiwykg qirdlyhyit syvvdgqiii ygpaysgret aysnaslliq nvtredagsy |
| 121 | tlhiikrgdg trgvtgyftf tlyletpkps isssnlnpre ametviltcd petpdtsyqw |
| 181 | wmnggslpmt hrfqlsetnr tlflfgvtky tagpyeceir nsgsasrsdp vtlnllhgpd |
| 241 | lprihpsytn yrsgdnlyls cfansnppaq yswtingkfq gsgqnlfipq ittkhsglyv |
| 301 | csvrnsatge esstsltvkv sastrigllp llnpt |

An exemplary human PSG2 nucleic acid sequence is set forth below (SEQ ID NO: 18; GenBank Accession No: NM_031246.3, Version 3, incorporated herein by reference):

| | |
|---|---|
| 1 | gacagagagg tgtcctgggc ctgaccccac ccatgagcct gggaattgct gctgccccag |
| 61 | gaagaggctc agtgcagaag gaggaaggac agcacagctg acagccgtgc tcaggaagtt |
| 121 | tctggatcct aggctcatct ccacagagga gaacacacag gcagcagaga ccatggggcc |
| 181 | cctctcagcc cctccctgca cagagcacat caaatggaag gggctcctgg tcacagcatc |
| 241 | acttttaaac ttctggaacc tgcccaccac tgcccaagtc acgattgaag cccagccacc |
| 301 | aaaagtttcc gaggggaagg atgttcttct acttgtccac aatttgcccc agaatcttac |
| 361 | tggctacatc tggtacaaag ggcaaatcag ggacctctac cattacatta catcatatgt |
| 421 | agtagacggt caaataatta tatatgggcc tgcatatagt ggacgagaaa cagcatattc |
| 481 | caatgcatcc ctgctgatcc agaatgtcac ccgggaggac gcaggatcct acaccttaca |
| 541 | catcataaag cgaggtgatg ggactagagg agtaactgga tatttcacct tcaccttata |
| 601 | cctggagact cccaagccct ccatctccag cagcaactta aaccccaggg aggccatgga |
| 661 | aactgtgatc ttaacctgtg atcctgagac tccggacaca agctaccagt ggtggatgaa |
| 721 | tggtcagagc ctccctatga ctcataggtt tcagctgtcc gaaaccaaca ggaccctctt |
| 781 | tctatttggt gtcacaaagt atactgcagg accctatgaa tgtgaaatac ggaactcagg |
| 841 | gagtgccagc cgcagtgacc cagtcaccct gaatctcctc catggtccag acctccccag |
| 901 | aattcaccct tcatacacca attaccgttc aggagataac ctctacttgt cttgcttcgc |
| 961 | gaactctaac ccaccggcac agtattcttg gacaattaat gggaagtttc agcaatcagg |
| 1021 | acaaaatctg tttatccccc aaattactac aaagcatagc gggctctatg tttgctctgt |
| 1081 | tcgtaactca gccactggcg aggaaagctc cacatcgttg acagtcaaag tctctgcttc |
| 1141 | tacaagaata ggacttcttc ctctccttaa tccaacatag cagctgtgat gtcatttctg |
| 1201 | tatttcagga agactggcag gagatttatg gaaaggtctc ttacaaggac tcttgaatac |
| 1261 | aagctcctga taacttcaag atcataccac tggactaaga actttcaaaa ttttaatgaa |
| 1321 | caggctgata ccttcatgaa attcaagaca aagaagaaaa atactcaatg ttattggact |
| 1381 | aaataatcaa aaggataatg atttcataat tttctatttg aaaatgtgct gattcttgga |
| 1441 | atgtttcatt ctccagattt atgaacattt tttcttgagc aattggtaaa gtatactttt |
| 1501 | gtaaacaaaa attgaaacat ttccttttgc tctctatctg agtgccccag aattgggaat |
| 1561 | ctattcatga gtattcatat gtttatggta ataaagctat ttgcacaagt tc |

An exemplary human PSG4 amino acid sequence is set forth below (SEQ ID NO: 19; GenBank Accession No: AAH08405.1, Version 1, incorporated herein by reference):

| | |
|---|---|
| 1 | mgtlsappct qrikwkglll tasllnfwnl pttaqvtiea eptkvsegkd vlllvhnlpq |
| 61 | nltgyiwykg qmrdlyhyit syvvdgeiii ygpayggret aysnaslliq nvtredagsy |
| 121 | tlhiikgddg trgvtgrftf tlhletpkps isssnlnpre tmeaysltcd petpdasylw |
| 181 | wmngqslpmt hslklsetnr tlfllgvtky tagpyeceir npvsasrsdp vtlnllpkls |
| 241 | kpyitinnln prenkdvltf tcepksenyt yiwwlngqsl pvsprvkrpi enrililpnv |
| 301 | trnetgpyqc eirdryggir sdpvtlnvly gpdlpsiyps ftyyrsgenl ylscfaesnp |
| 361 | raqyswting kfqlsgqkls ipqittkhsg lyacsvrnsa tgkessksit vkvsdwilp |

An exemplary human PSG4 nucleic acid sequence is set forth below (SEQ ID NO: 20; GenBank Accession No: M94891.1, Version 1, incorporated herein by reference):

| | |
|---|---|
| 1 | ggacagcaca gctgacagcc gtactcagga agcttctgga tcctaggctt atctccacag |
| 61 | aggagaacac acaagcagca gagaccatgg ggcccctctc agcccctccc tgcacacacc |
| 121 | tcatcacttg gaaggggctc ctgctcacag catcactttt aaacttctgg aatccgccca |
| 181 | caactgccca agtcacgatt gaagcccagc cacccaaagt ttctgagggg aaggatgttc |
| 241 | ttctacttgt ccacaatttg ccccagaatc ttgctggcta catttggtac aaagggcaaa |
| 301 | tgacatacgt ctaccattac attacatcat atgtagtaga cggtcaaaga attatatatg |
| 361 | ggcctgcata cagtggaaga gaaagagtat attccaatgc atccctgctg atccagaatg |
| 421 | tcacgcagga ggatgcagga tcctacacct tacacatcat aaagcgacgc gatgggactg |
| 481 | gaggagtaac tggacatttc accttcacct tacacctgga gactcccaag ccctccatct |
| 541 | ccagcagcaa cttaaatccc agggaggcca tggaggctgt gatcttaacc tgtgatcctg |
| 601 | cgactccagc cgcaagctac cagtggtgga tgaatggtca gagcctccct atgactcaca |
| 661 | ggttgcagct gtccaaaacc aacaggaccc tctttatatt tggtgtcaca aagtatattg |
| 721 | caggacccta tgaatgtgaa atacggaacc cagtgagtgc cagccgcagt gacccagtca |
| 781 | ccctgaatct cctcccaaag ctgtccaagc cctacatcac aatcaacaac ttaaacccca |
| 841 | gagagaataa ggatgtctta accttcacct gtgaacctaa gagtgagaac tacacctaca |
| 901 | tttggtggct aaatggtcag agcctccctg tcagtcccag ggtaaagcga cccattgaaa |
| 961 | acaggatcct cattctaccc aatgtcacga gaaatgaaac aggaccttat caatgtgaaa |
| 1021 | tacgggaccg atatggtggc atccgcagtg acccagtcac cctgaatgtc ctctatggtc |
| 1081 | cagacctccc cagcatttac ccttcattca cctattaccg ttcaggagaa aacctctact |
| 1141 | tgtcctgctt cgccgagtct aacccacggg cacaatattc ttggacaatt aatgggaagt |
| 1201 | ttcagctatc aggacaaaag ctctctatcc cccaaataac tacaaagcat agtgggctct |
| 1261 | atgcttgctc tgttcgtaac tcagccactg gcaaggaaag ctccaaatcc atcacagtca |
| 1321 | aagtctctga ctggatatta ccctgaattc tactagttcc tccaattcca ttttctccca |
| 1381 | tggaatcacg aagagcaaga cccactctgt tccagaagcc ctataagctg gaggtggaca |
| 1441 | actcgatgta aatttcatgg gaaaaccctt gtacctgaca tgtgagccac tcagaactca |
| 1501 | ccaaaatgtt cgacaccata acaacagcta ctcaaactgt aaaccaggat aagaagttga |
| 1561 | tgacttcaca ctgtggacag tttttccaaa gatgtcagaa caagactccc catcatgata |
| 1621 | aggctcccac ccctcttaac tgtccttgct catgcctgcc tctttcactt ggcaggataa |
| 1681 | tgcagtcatt agaatttcac atgtagtagc ttctgagggt aacaacagag tgtcagatat |
| 1741 | gtcatctcaa cctcaaactt ttacgtaaca tctcagggga aatgtggctc tctccatctt |
| 1801 | gcatacaggg ctcccaatag aaatgaacac agagatattg cctgtgtgtt tgcagagaag |
| 1861 | atggtttcta taaagagtag gaaagctgaa attatagtag agtctccttt aaatgcacat |
| 1921 | tgtgtggatg gctctcacca tttcctaaga gatacagtgt aaaacgtgac agtaatactg |
| 1981 | attctagcag aataaaacat gtaccacatt tgctaaaaaa aaaaaaaaaa aaaaaaaaaa |
| 2041 | aaa |

An exemplary human PSG5 amino acid sequence is set forth below (SEQ ID NO: 21; GenBank Accession No: AAH12607.1, Version 1, incorporated herein by reference):

| | |
|---|---|
| 1 | mgplsappct qhitwkgvll tasllnfwnl pitaqvtiea lppkvsegkd vlllvhnlpq |
| 61 | nlagyiwykg qlmdlyhyit syvvdgqini ygpaytgret vysnaslliq nvtredagsy |
| 121 | tlhiikrgdr trgvtgyftf nlylklpkpy itinnskpre nkdvlaftce pksenytyiw |
| 181 | wlngqslpvs prvkqpienr ililpsvtrn etgpyeceir drdggmhsdp vtlnvlygpd |
| 241 | lpsiypsfty yrsgenlyls cfaesnppae yfwtingkfq qsgqklsipq ittkhrglyt |
| 301 | csvrnsatgk essksmtvev sapsgigrlp llnpi |

An exemplary human PSG5 nucleic acid sequence is set forth below (SEQ ID NO: 22; GenBank Accession No: BC012607.1, Version 1, incorporated herein by reference):

| | |
|---|---|
| 1 | ggggaaggag gaaggacagc acagcctaca gccgtgctca ggaagtttct ggatcctagg |
| 61 | ctcagctcca cagaggagaa cacgcaggcg cagagaccat ggggcccctc tcagcccctc |
| 121 | cctgcacaca gcacatcacc tggaaggggg tcctgctcac agcatcactt ttaaacttct |
| 181 | ggaacctgcc tatcactgct caagtcacga ttgaagccct gccacccaaa gtttccgagg |
| 241 | ggaaggatgt tcttctactt gtccacaatt tgcctcagaa tcttgctggc tacatctggt |
| 301 | acaaaggaca actgatggac ctctaccatt acattacatc atatgtagta gacggtcaaa |
| 361 | taaatatata tgggcctgca tacactggac gagaaacagt atattccaat gcatccctgc |
| 421 | tgatccagaa tgtcacccgg gaagacgcag gatcctatac cttacacatc ataaagcgag |
| 481 | gtgataggac tagaggagta actggatatt tcaccttcaa cttatacctg aagctgccca |
| 541 | agccctacat caccatcaac aactcaaaac ccagggagaa taaggatgtc ttagccttca |
| 601 | cctgtgaacc taagagtgag aactacacct acatttggtg gctaaatggt cagagcctcc |
| 661 | cggtcagtcc cagggtaaag caacccattg aaaacaggat cctcattcta cccagtgtca |
| 721 | cgagaaatga aacaggaccc tatgaatgtg aaatacggga ccgagatggt ggcatgcaca |
| 781 | gtgacccagt caccctgaat gtcctctatg gtccagacct ccccagcatt tacccttcat |
| 841 | tcacctatta ccgttcagga gaaaacctct acttgtcctg cttcgcggaa tctaacccac |
| 901 | cggcagagta tttttggaca attaatggga agtttcagca atcaggacaa aagctctcta |
| 961 | tcccccaaat tactacaaag catagagggc tctatacttg ctctgttcgt aactcagcca |
| 1021 | ctggcaagga aagctccaaa tccatgacag tcgaagtctc tgctccttca ggaataggac |
| 1081 | gtcttcctct ccttaatcca atatagcagc cgtgaagtca tttctgtatt tcaggaagac |
| 1141 | tggcagacag ttgctttgat tcttcctcaa actacttaca atcacctaca gtccaaaatt |
| 1201 | gctttttctt caaggagatt tatggaaaag actctgacaa ggactcttga atacaagttc |
| 1261 | ctgataactt caagatcata ccactggact aagaactttc aaaattttaa tgaacaggct |
| 1321 | gataccttca tgaaattcta gacaaagaag aaaaaaactc catgttattg gactaaataa |
| 1381 | tcaaaagcat aatgttttca taattttcta tttgaaaatg tgctgattct ttgaatgttt |
| 1441 | tattctccag atttatgaac tttttttctt gagcaattgg taaagtatac ttttgtaaac |
| 1501 | aaaaattgaa acatttgctt ttactctcta tctgagtgcc ccagaattgg gaaactattc |
| 1561 | atgagtattc atatgtttat ggtaataaag ttatctgcac aagttcaaaa aaaaaaaaaa |
| 1621 | aaaaaaaaaa aaaaaa |

An exemplary human PSG6 amino acid sequence is set forth below (SEQ ID NO: 23; GenBank Accession No: AAC25619.1, Version 1, incorporated herein by reference):

| | |
|---|---|
| 1 | mgplsappct qhitwkglll tasllnfwnl pttaqviiea kppkvsegkd vlllvhnlpq |
| 61 | nltgyiwykg qmtdlyhyit syvvhgqiiy gpaysgretv ysnaslliqn vtqedagsyt |
| 121 | lhiikrgdgt ggvtgyftvt lysetpkpsi sssnlnprev meavrlicdp etpdasylwl |
| 181 | lngqnlpmth rlqlsktnrt lylfgvtkyi agpyeceirn pvsasrsdpv tlnllpklpm |
| 241 | pyitinnlnp rekkdvlaft cepksrnyty iwwlngqslp vsprvkrpie nrililpsvt |
| 301 | rnetgpyqce irdryggirs npvtlnvlyg pdlpriypsf tyyrsgenld lscfadsnpp |
| 361 | aeyswtingk fqlsgqklfi pqittnhsgl yacsvrnsat gkeisksmiv kvsetaspqv |
| 421 | tyagpntwfq eilll |

An exemplary human PSG6 nucleic acid sequence is set forth below (SEQ ID NO: 24; GenBank Accession No: M33666.1, Version 1, incorporated herein by reference):

| | |
|---|---|
| 1 | gggcgggcct aggctcatct ccacagggga gaacacacag acagcagaga ccatgggacc |
| 61 | cctctcagcc cctccctgca ctcagcacat cacctggaag gggctcctgc tcacagcatc |
| 121 | acttttaaac ttctggaacc tgcccaccac tgcccaagta ataattgaag ccaagccacc |
| 181 | caaagtttcc gaggggaagg atgttcttct acttgtccac aatttgcccc agaatcttac |
| 241 | tggctacatc tggtacaaag ggcaaatgac ggacctctac cattacatta catcatatgt |
| 301 | agtacacggt caaattatat atgggcctgc ctacagtgga cgagaaacag tatattccaa |
| 361 | tgcatccctg ctgatccaga atgtcacaca ggaggatgca ggatcctaca ccttacacat |
| 421 | cataaagcga ggcgatggga ctggaggagt aactggatat ttcactgtca ccttatactc |
| 481 | ggagactccc aagccctcca tctccagcag caacttaaac cccagggagg tcatggaggc |
| 541 | tgtgcgctta atctgtgatc ctgagactcc ggatgcaagc tacctgtggt tgctgaatgg |
| 601 | tcagaacctc cctatgactc acaggttgca gctgtccaaa accaacagga ccctctatct |
| 661 | atttggtgtc acaaagtata ttgcaggacc ctatgaatgt gaaatacgga acccagtgag |
| 721 | tgccagccgc agtgacccag tcaccctgaa tctcctcccg aagctgccca tgccttacat |
| 781 | caccatcaac aacttaaacc ccagggagaa gaaggatgtg ttagccttca cctgtgaacc |
| 841 | taagagtcgg aactacacct acatttggtg gctaaatggt cagagcctcc cggtcagtcc |
| 901 | gagggtaaag cgacccattg aaaacaggat actcattcta cccagtgtca cgagaaatga |
| 961 | aacaggaccc tatcaatgtg aaatacggga ccgatatggt ggcatccgca gtaacccagt |
| 1021 | caccctgaat gtcctctatg gtccagacct ccccagaatt tacccttcat tcacctatta |
| 1081 | ccgttcagga gaaaacctcg acttgtcctg ctttgcggac tctaacccac cggcagagta |
| 1141 | ttcttggaca attaatggga agtttcagct atcaggacaa aagctcttta tcccccaaat |
| 1201 | tactacaaat catagcgggc tctatgcttg ctctgttcgt aactcagcca ctggcaagga |
| 1261 | aatctccaaa tccatgatag tcaaagtctc tgagacagca tctccccagg ttacctatgc |
| 1321 | tggtccaaac acctggtttc aagaaatcct tctgctgtga cctcccaaag tgctaggatt |
| 1381 | aaaacatgac ccaccatgaa acccgccca |

An exemplary human GABRA3 amino acid sequence is set forth below (SEQ ID NO: 25; GenBank Accession No: AAG12455.1, Version 1, incorporated herein by reference):

| | |
|---|---|
| 1 | miitqtshcy mtslgilfli nilpgttgqg esrrqepgdf vkqdigglsp khapdipdds |
| 61 | tdnitiftri ldrlldgydn rlrpglgdav tevktdiyvt sfgpvsdtdm eytidvffrq |
| 121 | twhderlkfd gpmkilplnn llaskiwtpd tffhngkksv ahnmttpnkl lrlvdngtll |
| 181 | ytmrltihae cpmhledfpm dvhacplkfg syayttaevv yswtlgknks vevaqdgsrl |
| 241 | nqydllghvv gteiirsstg eyvvmtthfh lkrkigyfvi qtylpcimtv ilsqvsfwln |
| 301 | resvpartvf gvttvltmtt lsisarnslp kvayatamdw fiavcyafvf saliefatvn |
| 361 | yftkrswawe gkkvpealem kkktpaapak ktsttfnivg ttypinlakd tefstiskga |
| 421 | apsasstpti iaspkatyvq dsptetktyn syskvdkisr iifpvlfaif nlvywatyvn |
| 481 | resaikgmir kg |

An exemplary human GABRA3 nucleic acid sequence is set forth below (SEQ ID NO: 26; GenBank Accession No: NM_000808.3, Version 3, incorporated herein by reference):

| | |
|---|---|
| 1 | gagagagaga gagagagaga gagagagaga gagcgagaga gcgtgagcgc gcgcaagcta |
| 61 | gcgagcaaac cagagagaca gaccgagaga gggaccagga gagagaccca gagagagaag |
| 121 | aagaagccag aagccgagct ctgtcagggc tcaacctcca acttgtttca gttcattcat |
| 181 | ccttctctcc tttccgctca gactgtagag ctcggtctct ccaagtttgt gcctaagaag |
| 241 | atgataatca cacaaacaag tcactgttac atgaccagcc ttgggattct tttcctgatt |
| 301 | aatattctcc ctggaaccac tggtcaaggg gaatcaagac gacaagaacc cggggacttt |
| 361 | gtgaagcagg acattggcgg gctgtctcct aagcatgccc cagatattcc tgatgacagc |
| 421 | actgacaaca tcactatctt caccagaatc ttggatcgtc ttctggacgg ctatgacaac |
| 481 | cggctgcgac ctgggcttgg agatgcagtg actgaagtga agactgacat ctacgtgacc |
| 541 | agttttggcc ctgtgtcaga cactgacatg gagtacacta ttgatgtatt ttttcggcag |
| 601 | acatggcatg atgaaagact gaaatttgat ggccccatga agatccttcc actgaacaat |
| 661 | ctcctggcta gtaagatctg gacaccggac accttcttcc acaatggcaa gaaatcagtg |
| 721 | gctcataaca tgaccacgcc caacaagctg ctcagattgg tggacaacgg aaccctcctc |
| 781 | tatacaatga ggttaacaat tcatgctgag tgtcccatgc atttggaaga ttttcccatg |
| 841 | gatgtgcatg cctgcccact gaagtttgga agctatgcct atacaacagc tgaagtggtt |
| 901 | tattcttgga ctctcggaaa gaacaaatcc gtggaagtgg cacaggatgg ttctcgcttg |
| 961 | aaccagtatg accttttggg ccatgttgtt gggacagaga taatccggtc tagtacagga |
| 1021 | gaatatgtcg tcatgacaac ccacttccat ctcaagcgaa aaattggcta ctttgtgatc |
| 1081 | cagacctact tgccatgtat catgactgtc attctgtcac aagtgtcgtt ctggctcaac |
| 1141 | agagagtctg ttcctgcccg tacagtcttt ggtgtcacca ctgtgcttac catgaccacc |
| 1201 | ttgagtatca gtgccagaaa ttccttacct aaagtggcat atgcgacggc catggactgg |
| 1261 | ttcatagccg tctgttatgc ctttgtattt tctgcactga ttgaatttgc cactgtcaac |
| 1321 | tatttcacca agcggagttg ggcttgggaa ggcaagaagg tgccagaggc cctggagatg |
| 1381 | aagaagaaaa caccagcagc cccagcaaag aaaaccagca ctaccttcaa catcgtgggg |
| 1441 | accacctatc ccatcaacct ggccaaggac actgaatttt ccaccatctc caagggcgct |
| 1501 | gctcccagtg cctcctcaac cccaacaatc attgcttcac ccaaggccac ctacgtgcag |
| 1561 | gacagcccga ctgagaccaa gacctacaac agtgtcagca aggttgacaa aatttcccgc |
| 1621 | atcatctttc ctgtgctctt tgccatattc aatctggtct attgggccac atatgtcaac |
| 1681 | cgggagtcag ctatcaaggg catgatccgc aaacagtaga tagtggtggc agtgcagcaa |
| 1741 | ccagagcact gtataccccg tgaagcatcc aggcacccaa accccggggc tccccttcgc |
| 1801 | gtatttcagg attctccttt ttacccctct accaagctgt gaccctcaat tcatatttat |
| 1861 | gaatctctac gcaaaaaata actacagaaa aattacttgt ccctccaata ttgcccagta |
| 1921 | taaccccatc aaagccaaac actgccattt gtccagttgc tcatcttagt ctgccaatct |
| 1981 | cccctagctg agggcactgc atgtatttta ttgcactctg cccgctgcaa aaagaacaag |
| 2041 | agattctact ctccatagtg gaagccttgg ctgtttgaga ggcccagaac aaggagaatt |
| 2101 | gttgactccc atctagatca gatgactcta acttactagg cagccaggtt aggctaggcc |
| 2161 | atgtgatcct gcgtgccacc tcccctgcct tcagcaaggc ctactaggca taagtactga |
| 2221 | tagcaaaggt gggagccagt tctacacccc caacccattt attggtttgg aaattagtgg |
| 2281 | ggacaattgg tactaaccac cgtctaccat gtatggccaa aataaataga actagctctg |
| 2341 | ccagcctggc accaagatgg ctggtgccct gccatgtcca gcccctcggg aaaatagtcc |
| 2401 | cctccttggt acatctctcc tccagaaaat cttcttcccc cactgccttt ggcacccttg |
| 2461 | tagccaactg agcactactt aatttggact cattaccacc tgtaaacttt tcaggaaaaa |
| 2521 | atgatcaagc attttttatt tatatcgaaa agttgcaaat agaaacaaag tgatctagat |
| 2581 | ttaaaaaaaa cattttttta aaatatggga gagatacaaa agtcacctcc ctgccaaggc |
| 2641 | aactagccta tactggattg ggtaagaggt ttggagtgga tggtagttga ggattgaagt |
| 2701 | ctggctcaaa agagaaggct actggcagat gaaagtcaaa ttcttccttc catacactcc |
| 2761 | acattccaca ccctggccca ggcac |

An exemplary human GABRB1 amino acid sequence is set forth below (SEQ ID NO: 27; GenBank Accession No: AAH22449.1, Version 1, incorporated herein by reference):

| | |
|---|---|
| 1 | mwtvqnresl gllsfpvmit mvccahstne psnmsyvket vdrllkgydi rlrpdfggpp |
| 61 | vdvgmridva sidmvsevnm dytltmyfqq swkdkrlsys giplnltldn rvadqlcvpd |
| 121 | tyflndkksf vhgvtvknrm irlhpdgtvl yglritttaa cmmdlrrypl deqnctleie |
| 181 | sygyttddie fywnggegav tgvnkielpq fsivdykmvs kkvefttgay prlslsfrlk |
| 241 | rnigyfilqt ympstlitil swvsfwinyd asaarvalgi ttvltmttis thlretlpki |
| 301 | pyvkaidiyl mgcfvfvfla lleyafvnyi ffgkgpqkkg askqdqsane knklemnkvq |
| 361 | vdahgnills tleirnetsg sevltsysdp katmysydsa siqyrkplss reaygraldr |
| 421 | hgvpskgrnr rrasqlkvki pdltdvnsid kwsrmffpit fslfnvvywl yyvh |

An exemplary human GABRB1 nucleic acid sequence is set forth below (SEQ ID NO: 28; GenBank Accession No: NM_000812.3, Version 3, incorporated herein by reference):

| | |
|---|---|
| 1 | agccttagcc agatcactga gcgcccagta aaaaaaacaa aatcaggttg agggcagaaa |
| 61 | tgaaatcaac atagcaacct ccaatgcatg aaggaaactc cgtttacaca tgctcgtagg |
| 121 | atcccctgcg tggaaacagc agcttgtctc tgactacccg gaggacatgg agcaccccaa |
| 181 | ataggaactt tagagggatt gaaatctgtt gcctgttcca ctaggaatat tgtttgcaag |
| 241 | gcacaaggtg tcttttggta gtgagcgcgc tctgcgcatg cgcaggtcca ttcgggaatt |
| 301 | actgcccagc agccgactaa gttgcattcc ttgaatcttc gcagaaaaga caattctttt |
| 361 | aatcagagtt agtaatgtgg acagtacaaa atcgagagag tctggggctt ctctctttcc |
| 421 | ctgtgatgat taccatggtc tgttgtgcac acagcaccaa tgaacccagc aacatgtcat |
| 481 | acgtgaaaga gacagtggac agattgctca aaggatatga cattcgcttg cggccggact |
| 541 | tcggagggcc ccccgtcgac gttgggatgc ggatcgatgt cgccagcata gacatggtct |
| 601 | ccgaagtgaa tatggattat acactcacca tgtatttcca gcagtcttgg aaagacaaaa |
| 661 | ggctttctta ttctggaatc ccactgaacc tcaccctaga caatagggta gctgaccaac |
| 721 | tctgggtacc agacacctac tttctgaatg acaagaaatc atttgtgcat ggggtcacag |
| 781 | tgaaaaatcg aatgattcga ctgcatcctg atggaacagt tctctatgga ctccgaatca |
| 841 | caaccacagc tgcatgtatg atggatcttc gaagatatcc actggatgag cagaactgca |
| 901 | ccctggagat cgaaagttat ggctatacca ctgatgacat tgaattttac tggaatggag |
| 961 | gagaaggggc agtcactggt gttaataaaa tcgaacttcc tcaattttca attgttgact |
| 1021 | acaagatggt gtctaagaag gtggagttca caacaggagc gtatccacga ctgtcactaa |
| 1081 | gttttcgtct aaagagaaac attggttact tcattttgca aacctacatg ccttctacac |
| 1141 | tgattacaat tctgtcctgg gtgtcttttt ggatcaacta tgatgcatct gcagccagag |
| 1201 | tcgcactagg aatcacgaca gtgcttacaa tgacaaccat cagcacccac ctcagggaga |
| 1261 | ccctgccaaa gatcccttat gtcaaagcga ttgatattta tctgatgggt tgctttgtgt |
| 1321 | ttgtgttcct ggctctgctg gagtatgcct ttgtaaatta catcttcttt gggaaaggcc |
| 1381 | ctcagaaaaa gggagctagc aaacaagacc agagtgccaa tgagaagaat aaactggaga |
| 1441 | tgaataaagt ccaggtcgac gcccacggta acattctcct cagcaccctg gaaatccgga |
| 1501 | atgagacgag tggctcggaa gtgctcacga gcgtgagcga ccccaaggcc accatgtact |
| 1561 | cctatgacag cgccagcatc cagtaccgca agcccctgag cagccgcgag gcctacgggc |
| 1621 | gcgccctgga ccggcacggg gtacccagca aggggcgcat ccgcaggcgt gcctcccagc |
| 1681 | tcaaagtcaa gatccccgac ttgactgatg tgaattccat agacaagtgg tcccgaatgt |
| 1741 | ttttccccat caccttttct ctttttaatg tcgtctattg gctttactat gtacactgag |
| 1801 | gtctgttcta atggttccat ttagactact ttcctcttct attgtttttt aaccttacag |
| 1861 | gtccccaaca gcgatactgc tgtttctcga ggtaagagat tcagccatcc aattggtttt |
| 1921 | aggtcttgca tatcagtttt attactgcac catgtttact tcaaaaagac aaaacaaaaa |
| 1981 | aaaaattatt tttccagtct accgtggtcc aggttatcag ctctttaaga gctctattaa |
| 2041 | ttgccatgtt tacaaacaaa cacaaagaga gaagttagac aggtagatct ttagcagtct |
| 2101 | tttctagttt ccctggattt cactgattta ttttttaggg aaaatgaaaa gaggaccttg |
| 2161 | ctgtccgcct gcactgcttc ctggtaaact ataacaaact tatgctgcca aaaaaaaaaa |
| 2221 | aaaaaa |

An exemplary human GABRB2 amino acid sequence is set forth below (SEQ ID NO: 29; GenBank Accession No: AAI05640.1, Version 1, incorporated herein by reference):

| | |
|---|---|
| 1 | mwrvrkrgyf giwsfpliia avcaqsvndp snmslvketv drllkgydir lrpdfggppv |
| 61 | avgmnidias idmvsevnmd ytltmyfqqa wrdkrlsynv iplnltldnr vadqlwvpdt |
| 121 | yflndkksfv hgvtvknrmi rlhpdgtvly glritttaac mmdlrrypld eqnctleies |
| 181 | ygyttddief ywrgddnavt gvtkielpqf sivdyklitk kvvfstgsyp rlslsfklkr |
| 241 | nigyfilqty mpsilitils wvsfwinyda saarvalgit tvltmttint hlretlpkip |
| 301 | yvkaidmylm gcfvfvfmal leyalvnyif fgrgpqrqkk aaekaasann ekmrldvnkm |
| 361 | dphenillst leiknemats eavmglgdpr stmlaydass iqyrkaglpr hsfgrnaler |
| 421 | hvaqkksrlr rrasqlkiti pdltdvnaid rwsriffpvv fsffnivywl yyvn |

An exemplary human GABRB2 nucleic acid sequence is set forth below (SEQ ID NO: 30; GenBank Accession No: NM_021911.2, Version 2, incorporated herein by reference):

| | |
|---|---|
| 1 | gcgcggggaa gggaagaaga ggacgaggtg gcgcggagac cgcgggagaa cacagtgcct |
| 61 | ccggaggaaa tctgctcggt ccccggcagc cgccgcttcc cctttgatgt tttggtacgc |
| 121 | cgtgcgcatg cgcctcacat tagaattact gcactgggca gactaagttg gatctcctct |
| 181 | cttcagtgaa accctcaatt ccatcaaaaa ctaaagggat gtggagagtg cggaaaaggg |
| 241 | gctactttgg gatttggtcc ttccccttaa taatcgccgc tgtctgtgcg cagagtgtca |
| 301 | atgaccctag taatatgtcg ctggttaaag agacggtgga tagactcctg aaaggctatg |
| 361 | acattcgtct gagaccagat tttggaggtc cccccgtggc tgtggggatg aacattgaca |
| 421 | ttgccagcat cgatatggtt tctgaagtca atatggatta taccttgaca atgtactttc |
| 481 | aacaagcctg gagagataag aggctgtcct ataatgtaat acctttaaac ttgactctgg |
| 541 | acaacagagt ggcagaccag ctctgggtgc ctgataccta tttcctgaac gataagaagt |
| 601 | catttgtgca cggagtgact gttaagaacc gcatgattcg cctgcatcct gatggcaccg |
| 661 | tcctttatgg actcagaatc acaaccacag ctgcctgcat gatggaccta aggaggtacc |
| 721 | cactggatga acaaaactgc accttggaaa ttgagagcta tggatacaca actgatgaca |
| 781 | ttgagtttta ctggcgtggc gatgataatg cagtaacagg agtaacgaaa attgaacttc |
| 841 | cacagttctc tattgtagat tacaaactta tcaccaagaa ggttgttttt tccacaggtt |
| 901 | cctatcccag gttatccctc agctttaagc ttaagagaaa cattggctac tttatcctgc |
| 961 | aaacatacat gccttccatc ctgattacca tcctctcctg ggtctccttc tggattaatt |
| 1021 | acgatgcttc agctgcaagg gtggcattag gaatcacaac tgtcctcaca atgaccacaa |
| 1081 | tcaacaccca cctccgggaa actctcccta aaatccccta tgtgaaggcc attgacatgt |
| 1141 | acctgatggg gtgctttgtc ttcgttttca tggcccttct ggaatatgcc ctagtcaact |
| 1201 | acatcttctt tgggaggggg ccccaacgcc aaaagaaagc agctgagaag gctgccagtg |
| 1261 | ccaacaatga gaagatgcgc ctggatgtca acaagatttt ttataaagat attaaacaaa |
| 1321 | atgggaccca atatcgatcc ttgtgggacc ctactggaaa cctctcccca actagacgga |
| 1381 | ctaccaatta cgatttctct ctgtatacga tggaccccca tgagaacatc ttactgagca |
| 1441 | ctctcgagat aaaaaatgaa atggccacat ctgaggctgt gatgggactt ggagacccca |
| 1501 | gaagcacaat gctagcctat gatgcctcca gcatccagta tcggaaagct gggttgccca |
| 1561 | ggcatagttt tggccgaaat gctctggaac gacatgtggc gcaaaagaaa agtcgcctga |
| 1621 | ggagacgcgc ctcccaactg aaaatcacca tccctgactt gactgatgtg aatgccatag |
| 1681 | atcggtggtc ccgcatattc ttcccagtgg ttttttcctt cttcaacatc gtctattggc |
| 1741 | tttattatgt gaactaaaac atggcctccc actggaagca aggactagat tcctcctcaa |
| 1801 | accagttgta cagcctgatg taggacttgg aaaacacatc aatccaggac aaaagtgacg |
| 1861 | ctaaaatacc ttagttgctg gcctatcctg tggtccattt cataccattt gggttgcttc |
| 1921 | tgctaagtaa tgaatacact aaggtccttg tggttttcca gttaaaacgc aagtgatttt |
| 1981 | tacacatggt ggctagacag cctgagtgct ccatgttgtc tgtttagtat gcagcctact |
| 2041 | cagaggggaa taatttggaa gatattctta gaaggctcaa cagcattgtc agtcatttgg |
| 2101 | tcataaccag atactcctca tcttcctttt taaggtcggc atgctgttga gatggcattg |
| 2161 | tccttatgaa acattatctg taatgccatg caaacacatt acttgaagat gatctatgat |
| 2221 | tttcttactg agatgataat ggtgtcatgg gaagattatt cccactggtt tctaaaagat |
| 2281 | tctcttgtga tttgagcagg ctaggggaat caagacagag actcaggaaa ccgatttggc |
| 2341 | cagtcccttt gcttgagcac ggagaatcaa ctatgatgtc tttcattttg gcatttagag |
| 2401 | atgaggagac ttctggacaa tatcatgcat ggtggccaag aaaatttttg ctaaacggct |
| 2461 | atttcctagg tggttaaagt aagagttaac aacacagagc agagtagaaa atcagaaacg |
| 2521 | acggttgtaa caaaaagcaa gcaagcaaga aagcatgacc cttatcagct ccacacagca |
| 2581 | tctggtctat acatactgtg tctagtggca gagggtgagt aagagagatt gtttttccaa |
| 2641 | catcactagc ttttgacagc atacctaaag aggtttttat agtttaatcg aagataacca |
| 2701 | tgaaatacaa aaattagaga aatgagaatt gtcttagata ttcaggttag attcagagtc |
| 2761 | attcaaatat acaaatacag agcacaaact agcaaacaga ttgtttttct cagacgaagt |
| 2821 | cagctgtccc tgcaattttt gtccctataa ttttatttct taatttaata gagtatttca |
| 2881 | agagaaggat ttgttcatca ctttaaagga ttcatggtcc tctccagctt tttttttgct |
| 2941 | tccaaaattc tgattatatt cttaggagac tggcaaagta aggctttgga gagattgagt |
| 3001 | gaactatatt ttcttatatg caatgaaatt caccattgta tttttcaaag caaataatac |
| 3061 | ttcaaggagt atgaacacac agagagttaa tttgtgcatt cattggtttg gggcttgatt |
| 3121 | ttcttaagag tgctttcatt gtatatgttg caatctgcct aatcaaggaa aatatgaaaa |
| 3181 | ctagtggaaa ataataacaa agccacatta acatgataaa tgttatgagc ctcagttgat |
| 3241 | attgcagaaa aatatataac tgttaatcag gcaggcatga tgtatttttt ttctattttt |
| 3301 | aagttatgcc attgtgcttc tttctacact aattggacat aagcattcat atgttggaca |
| 3361 | agaggagaat aagctaatat gtcttccttc tatcaatata aggctcatat tttgcttttc |
| 3421 | ttatagaaaa tagtgaattg ctaaattttc cttaaagttg aatttgattg aactttttct |
| 3481 | tggaggatga ctttgctatt tctattttga aaacaaagaa agctcttctt tttagagtaa |
| 3541 | tatccctaat tgtgcatagc agccagtatc cacctgtgaa taatgtatgt tttcttgatt |
| 3601 | ccttgtttcc ttcatgattt agcagcttgg aatatgatgg agtactattg cccttgcatt |
| 3661 | ccagctgtcc aagaacaaca acaaaccttt atccttcagc tttctgaatt tgccccaaaa |
| 3721 | tggaatggta ttttgttttt caccagtgta ggaaaccttg gccattatat tttgattaca |
| 3781 | taatcttgag cacctctgag tatcatcttt gggcagaatc tgaccaagat tcttctcaca |
| 3841 | ggctcagagt agagattcct catgtttctc caggacacac tttctcctag gcaatcattt |
| 3901 | tggggcatca cactaagtaa agtgcctgcc ccttgtcaac agggggtaaa gtttgactaa |
| 3961 | aaaacatcag gaaggaacat attgataact caggggcaca actccaaaat gccattctga |
| 4021 | aaaggctctt ctagagtcaa ctcagagccc aagtgtagaa gtgggctctt atgtgcaatc |
| 4081 | actgtacaag ccataaaaat ctttgatatt gccatttatg attacaacaa aattttagat |
| 4141 | gtccaagaaa gtcaacagaa caatccagat caaagtgtag agaagcatct ttgcagcttg |
| 4201 | agcctcaaaa gcataaaacc aggaattcat ttctgctctc agtgatacaa gaaacaaggc |
| 4261 | aaaggaaaaa agatgggacc ttagaagcaa tgccctagag taaaaagcat acattggcac |
| 4321 | tcagtaacca cagagataga aacaactgac tcagcctctc cagaaataac agtagatgta |
| 4381 | gaatttgttc ttaaatatat gtttcgttga aatgaaatgc agtcatcaaa tgcgaggagg |
| 4441 | ggtgctagac ctgataagtc taggaagacc agtcagcttg atagccagtt gttaaactaa |
| 4501 | aggaccacaa aggtgtgttt agtagttctg atatgggatt actatcagtt tatacatatg |
| 4561 | tatagatttt cttttaatta taaacagcca gatagccagc aattaaggtg ttttttcatc |
| 4621 | tttgtgaaat tcatggacaa attctgactc taaaatgcac agtctacatg aaatctttga |
| 4681 | tctcaatctg tatatatata tattgtacat gaccgctagt aggtatgaaa tgcatttcag |
| 4741 | agttgaaaac tcatgcaaca taatcatgtg tttgcataag gaatgttaaa attctttcta |
| 4801 | ctacaattta ttaacagggg aaaagattca agcagtgcca tctagacctt ttctcttctc |
| 4861 | atttcttcaa aagcagtgct aagtaaaata ataataataa aaatggttta tgaaccaaaa |
| 4921 | gactttacat tctagcatta ggaacacaca caaaaaaatc tgtcagctta caaaagcaca |
| 4981 | acacataaag aaagagcagt ggaatgcgta atggggaaat gaatcatgtt taaagcaaga |
| 5041 | cttgcattaa attggcaact aagataattt ccattttcta taggtaaccg tgttccagtt |
| 5101 | taatgattac cttcatactc aacaagaaaa atggcaaagc aacatcatca aaataggtct |
| 5161 | tacaacatat ctacatgtat atatgtgtat atatagattc atataaacat acatacatat |
| 5221 | atatgcattc atatgttgga aaacaaaagg aataagctaa tatgttttat ttccttcatt |
| 5281 | aaaccagtat atgagtattt ttaatttggc aaacaaaaat caacatactg aaataagtaa |
| 5341 | tacagggtac tgagcatttc tctatcagca aatcaatgcc tacagttttt atgaaccatt |
| 5401 | gcacagttca actcgacaat agtactgtac cccagaacat cagaagttaa gtagttgtca |
| 5461 | cattgcttgt ctaattgctc tctttttcaa ggaaacaaat aacattgtcc attggtgagt |
| 5521 | caaaaacaaa catgtcttaa gaaaatgaaa aatgataaaa attcctccaa agaggaagaa |
| 5581 | aaaaaaaaaa ctaaacccca ccatgttggt tagggcaaaa tactaagtat tcacaactgt |
| 5641 | agcaatcatg ccatttggct aaatgtacag agtacatgta atgtgagcaa ccataacttt |
| 5701 | cactgaacgt gtgtaaatat taaaacagat ttcttaaata tttttaactt ctaatttgta |
| 5761 | ttttatggta aggcaatgtg ctaattccgt ttatggctta agttgatgtg ttataatatg |
| 5821 | taaataatta aacatgctgt aaatgaatag tcaatagata caaataattg tttagttttt |
| 5881 | ttcttaattt ttatttattt tttactaagt actattactg cagtgccccc agcagagtaa |
| 5941 | tttaaacacc cttttgcata aatggtttgg gaatcccaga cagaagggtt tcaatttgct |
| 6001 | atcacacaca cagtcacaca cagtcacaca cacacacaca cacacactca aacacacata |
| 6061 | atcttttgct cacccactct gtctctctgc ccgtctatgg gaaacactct actatttatg |
| 6121 | tagtcaatat aacaaacaag agatagcctt tgaaatggca attgggaaaa gcaaaaatga |
| 6181 | tcattgataa cttaatagac ttttttatat gtcagagttc aagaaaccaa agtttgcaac |
| 6241 | ttaaatctca gttttcaaac agtaaaagtt tgtatagatg gcactacaga aaaataaaaa |
| 6301 | tggttattcc taccagctcc agaaatgtgt tacacaatat acactagaag actttttttt |
| 6361 | taataatagt attttgtcct taggtagtat tgttcctttc aaccaaagac tccagaatta |
| 6421 | ttgaaaaaca aaaaaggaaa aaaaaaatga gataatgggg gaaaaaaagg acgctgcttc |
| 6481 | acaccaaagg tttaaaaaaa tattttccaa atgcactagg aacattgtaa aaaccagaaa |
| 6541 | ggagtgaaca gaatgctaat gaagatactg ccctaaagaa atctgtacaa aaaaaaaaaa |
| 6601 | aaaaagcagc actcttaaaa agctactagg tttttggaaa ttgaagtaaa atatttctgt |
| 6661 | agatatattc aaaattctga catcaaattt taatttgctt aaagaagttt tatttgtgat |
| 6721 | gaagatcaat atgattcaaa tgatgaaaaa caaaactgta tgtcctttga gttcccaaac |
| 6781 | caaatgtcac tgccgtgtca catcctctaa tctgatgccc atgtgtgtgt ccttgcgcca |
| 6841 | tttgaagcaa gttgacagtt ccatcagttt gtttttgttt ttgtttttgg ttcaataatt |
| 6901 | tgagttcaac catcgtcatt ttaaacataa ttaatcccaa caaagttgcc tatagtttaa |
| 6961 | tttaaacctc acagcatttt taatttattt tgcatgggtg tttaaatagt tctgttacga |
| 7021 | atattgtccc tgcacagttc taaattgtcc ttttataaaa aaaaaaagtg atattctagt |
| 7081 | ttcttcacaa tgtagtgatt atcagcctta ctaattacct tgtagccttt cttaatatgc |
| 7141 | acataatgca cattttccga tggctagaaa aatgcaaaac acagtggata tcattgcatc |
| 7201 | tattttcatg tctttctaaa aacaggacta ttaaaatctc tgggatacat gagatagtaa |
| 7261 | aaaatgagga ttataaatga atagcacata agaattattt tcttgaattt aaacttattg |
| 7321 | cagccagttt cagcatgtaa atatataata atgttggcta gtgtgtaatt cttgaactaa |
| 7381 | gaaatataaa taaaaataaa aagattgtg |

An exemplary human GABRG2 amino acid sequence is set forth below (SEQ ID NO: 31; GenBank Accession No: AAD50273.1, Version 1, incorporated herein by reference):

| | |
|---|---|
| 1 | msspniwstg ssvystpvfs qkmtvwilll lslypgftsq ksdddyedya snktwvltpk |
| 61 | vpegdvtvil nnllegydnk lrpdigvkpt lihtdmyvns igpvnainme ytidiffaqt |
| 121 | wydrrlkfns tikvlrinsn mvgkiwipdt ffrnskkada hwittpnrml riwndgrvly |
| 181 | tlrltidaec qlqlhnfpmd ehscplefss ygypreeivy qwkrssvevg dtrswrlyqf |
| 241 | sfvglrntte vvkttsgdyv vmsvyfdlsr rmgyftiqty ipctlivvls wvsfwinkda |
| 301 | vpartslgit tvltmttlst iarkslpkvs yvtamdlfvs vcfifvfsal veygtlhyfv |
| 361 | snrkpskdkd kkkknpapti dirprsatiq mnnathlqer deeygyecld gkdcasffcc |
| 421 | fedcrtgawr hgrihiriak mdsyariffp tafclfnlvy wvsylyl |

An exemplary human GABRG2 nucleic acid sequence is set forth below (SEQ ID NO: 32; GenBank Accession No: NM_198904.2, Version 2, incorporated herein by reference):

| | |
|---|---|
| 1 | gtaagtgtga ggggcatgag tatacacgag tgtgcgtgtc tttccctctc ccttatttgt |
| 61 | ctccgtatga gtctctcctt gtaccctccc cctgcctcga tgatattact cccccagact |
| 121 | tggaagccgc tgccagagtg acgctttgat ggtatctgca agcgtttttg ctgatcttat |
| 181 | ctctgccccc tgaatattaa ttccctaatc tggtagcaat ccatctcccc agtgaaggac |
| 241 | ctactagagg caggtggggg gagccaccat cagatcataa gcataagaat aatacaaagg |
| 301 | ggagggattc ttctgcaacc aagaggcaag aggcgagaga aggaaaaaaa aaaaagcgat |
| 361 | gagttcgcca aatatatgga gcacaggaag ctcagtctac tcgactcctg tattttcaca |
| 421 | gaaaatgacg gtgtggattc tgctcctgct gtcgctctac cctggcttca ctagccagaa |
| 481 | atctgatgat gactatgaag attatgcttc taacaaaaca tgggtcttga ctccaaaagt |
| 541 | tcctgagggt gatgtcactg tcatcttaaa caacctgctg gaaggatatg acaataaact |
| 601 | tcggcctgat ataggagtga agccaacgtt aattcacaca gacatgtatg tgaatagcat |
| 661 | tggtccagtg aacgctatca atatggaata cactattgat atattttttg cgcaaacgtg |
| 721 | gtatgacaga cgtttgaaat ttaacagcac cattaaagtc ctccgattga acagcaacat |
| 781 | ggtggggaaa atctggattc cagacacttt cttcagaaat tccaaaaaag ctgatgcaca |
| 841 | ctggatcacc acccccaaca ggatgctgag aatttggaat gatggtcgag tgctctacac |
| 901 | cctaaggttg acaattgatg ctgagtgcca attacaattg cacaactttc caatggatga |
| 961 | acactcctgc cccttggagt tctccagtta tggctatcca cgtgaagaaa ttgtttatca |
| 1021 | atggaagcga agttctgttg aagtgggcga cacaagatcc tggaggcttt atcaattctc |
| 1081 | atttgttggt ctaagaaata ccaccgaagt agtgaagaca acttccggag attatgtggt |
| 1141 | catgtctgtc tactttgatc tgagcagaag aatgggatac tttaccatcc agacctatat |
| 1201 | cccctgcaca ctcattgtcg tcctatcctg ggtgtctttc tggatcaata aggatgctgt |
| 1261 | tccagccaga acatctttag gtatcaccac tgtcctgaca atgaccaccc tcagcaccat |
| 1321 | tgcccggaaa tcgctcccca aggtctccta tgtcacagcg atggatctct ttgtatctgt |
| 1381 | ttgtttcatc tttgtcttct ctgctctggt ggagtatggc accttgcatt attttgtcag |
| 1441 | caaccggaaa ccaagcaagg acaaagataa aaagaagaaa aaccctcttc ttcggatgtt |
| 1501 | ttccttcaag gcccctacca ttgatatccg cccaagatca gcaaccattc aaatgaataa |
| 1561 | tgctacacac cttcaagaga gagatgaaga gtacggctat gagtgtctgg acggcaagga |
| 1621 | ctgtgccagt tttttctgct gttttgaaga ttgtcgaaca ggagcttgga gacatgggag |
| 1681 | gatacatatc cgcattgcca aaatggactc ctatgctcgg atcttcttcc ccactgcctt |
| 1741 | ctgcctgttt aatctggtct attgggtctc ctacctctac ctgtgaggag gtatgggttt |
| 1801 | tactgatatg gttcttattc actgagtctc atggagagat gtctgttcta agtccactta |
| 1861 | aataatcctc tatgtggttg ataatgatct gaatctgttt ctatgtccaa acctggtaaa |
| 1921 | ttttataatg tcatattgtt tgtgcccagc cctcctttgg ttagtgtact ttgaacttcg |
| 1981 | atgtttgctg tgtttcaaac ctgcaaggca aagtaaaatt agagcaagaa cattcaaacc |
| 2041 | aaataagata tttttcagct acagcaaata aaacagtgaa agccctgact atttacagta |
| 2101 | gtggtatcct tactagattc ataatgcaat tagatagaaa aggtccaaaa ctgtacccta |
| 2161 | tgttcactcc gggtcaagtt gtgataaatt tgatcccaat agaatacctc cctcatttaa |
| 2221 | gaaaaatcat aactcacttt aaatatgaaa gcctagtcca gaaatctatt acacctttat |
| 2281 | ctcaagatag gaagaaaatt tcctccacat tcatgtacaa tgatgtaaat atttcaataa |
| 2341 | cttagaatgc ttcaagttta gtgcatgcat ctctttagat ccaaaataaa tggactgaag |
| 2401 | ttatcatcct attgtctttt attttgtgtc cttgggctat aaaagattcc tgaatgtaat |
| 2461 | tataaggatt tgggtttgga aatggaggga ggaattttca ttgccttctc cctcatgcat |
| 2521 | gaagattcga acagcttatt ttttccttgt atgacatatt acaacacttt aagtaaaata |
| 2581 | tagactggat aatcaacatt tgccacctct aaatatgccc aatttcataa ctagagtata |
| 2641 | aagtaattgt atgtgcttgc cgctattttt ttcttccttt taggatgata gatcataaca |
| 2701 | gaacttattc tccatctcaa gatctgcttc tagtgattgt gagtgccttg tgggcagaat |
| 2761 | ccttgtcatt tcctctttgg gtctgtagca ccttgcatag tgcctggcat atagttggtg |
| 2821 | ctcaataaat atggtttgaa gtgaattgcc ctcacatgct tctggcaaat ctctgtgctg |
| 2881 | gcctgaaacc agtgactcat cttctcacat aggtgttgtc aagtgatatt tgattttgta |
| 2941 | aaaataacca gtaggatcca aagaacttta gctatttatg ttcatcttca aaaaattatt |
| 3001 | ttaggcaaag tccatactcc ttttaaaaca atatttatgt cctatgtttg tgtatagaca |
| 3061 | tgactctact agggcataat tagagtttgt gtattatttt tccaggtttg gggatgagtc |
| 3121 | agtccttgcc catccacaat tttgtttgtg aacttataac aggaataagc aaaattcata |
| 3181 | cctgactagt gttcagaatg tagcattctg tgcgaaaaag tattgaagat tagcttttaa |
| 3241 | aaactgaaaa aaaaaaatga atgactcaca tagaggttga gccaatgact gtggcctcat |
| 3301 | tagattacat tgtagttaaa caaagcaatt tctccagact taaaacatga tgagttgagc |
| 3361 | tctatcttca tgtactcatc ctgaatcctt atttttctaa aatagcaccc tttgttaatt |
| 3421 | atttttatgg aaattattac tctgtcataa ttaaatcata gctagtataa ctttacagat |
| 3481 | aacctaaaaa gaatagaaaa gaagagagag tggctttgtc agtataaaac catgtaaagt |
| 3541 | catcatcaag tcatctggat gaatcttgaa acacatttag ctgccagttt tacaaacctt |
| 3601 | taatatatca gtgctccagt atataacctc aaacaaatgt aaatagaacg aattattttc |
| 3661 | ttgttttgaa ttgtcaatat attaaatgtt gactctttgg gagagttgtt ggcaagtttc |
| 3721 | aatggtgaga aacattattg tcaacttgaa atgtgttctg taatggggac actacaaaaa |
| 3781 | gctagctttc caatgtgtgc atagtattgg caatatgaat atatattata tataatctaa |
| 3841 | tacttattat aagctgctcc ctgtctatgt atttggaaac cttttcacaa agggaattgc |
| 3901 | ctaacatgtg gacttttaca ataaaaatgc tgcattctaa tccatggtgg catctca |

An exemplary human GABRQ amino acid sequence is set forth below (SEQ ID NO: 33; GenBank Accession No: EAW99424.1, Version 1, incorporated herein by reference):

| | |
|---|---|
| 1 | mgirgmlraa villlirtwl aegnypspip kfhfefssav pevvlnlfnc kncaneavvq |
| 61 | kildrvlsry dvrlrpnfgg apvpvrisiy vtsieqisem nmdytitmff hqtwkdsrla |
| 121 | yyettlnltl dyrmheklwv pdcyflnskd afvhdvtven rvfqlhpdgt vrygirlttt |
| 181 | aacsldlhkf pmdkqacnlv vesygytved iilfwddngn aihmteelhi pqftflgrti |
| 241 | tskevyfytg syirlilkfq vqrevnsylv qvywptvltt itswisfwmn ydssaarvti |
| 301 | gltsmliltt idshlrdklp niscikaidi yilvclffvf lslleyvyin ylfysrgprr |
| 361 | qprrhrrprr viaryryqqv vvgnvqdgli nvedgvsslp itpaqaplas peslgsltst |
| 421 | seqaqlatse slspltslsg qaplatgesl sdlpstseqa rhsygvrfng fqaddsifpt |
| 481 | eirnrveahg hgvthdheds neslssderh ghgpsgkpml hhgekgvqea gwdlddnndk |
| 541 | sdclaikeqf kcdtnstwgl nddelmahgq ekdsssesed scppspgcsf tegfsfdlfn |
| 601 | pdyvpkvdkw srflfplafg lfnivywvyh my |

An exemplary human GABRQ nucleic acid sequence is set forth below (SEQ ID NO: 34; GenBank Accession No: NM_018558.3, Version 3, incorporated herein by reference):

| | |
|---|---|
| 1 | gcgcccagaa cgccccggcc atgggcatcc gaggcatgct gcgagccgca gtgatcctgc |
| 61 | tgctcatcag gacctggctc gcggagggca actaccccag tcccatcccg aaattccact |
| 121 | tcgagttctc ctctgctgtg cccgaagtcg tcctgaacct cttcaactgc aaaaattgtg |
| 181 | caaatgaagc tgtggttcaa aagattttgg acagggtgct gtcaagatac gatgtccgcc |
| 241 | tgagaccgaa ttttggaggt gcccctgtgc ctgtgagaat atctatttat gtcacgagca |
| 301 | ttgaacagat ctcagaaatg aatatggact acacgatcac gatgtttttt catcagactt |
| 361 | ggaaagattc acgcttagca tactatgaga ccaccctgaa cttgaccctg gactatcgga |
| 421 | tgcatgagaa gttgtgggtc cctgactgct actttctgaa cagcaaggat gctttcgtgc |
| 481 | atgatgtgac tgtggagaat cgcgtgtttc agcttcaccc agatggaacg gtgcggtacg |
| 541 | gcatccgact caccactaca gcagcttgtt ccctggatct gcataaattc cctatggaca |
| 601 | agcaggcctg caacctggtg gtagagagct atggttacac ggttgaagac atcatattat |
| 661 | tctgggatga caatgggaac gccatccaca tgactgagga gctgcatatc cctcagttca |
| 721 | ctttcctggg aaggacgatt actagcaagg aggtgtattt ctacacaggt tcctacatac |
| 781 | gcctgatact gaagttccag gttcagaggg aagttaacag ctaccttgtg caagtctact |
| 841 | ggcctactgt cctcaccact attacctctt ggatatcgtt ttggatgaac tatgattcct |
| 901 | ctgcagccag ggtgacaatt ggcttaactt caatgctcat cctgaccacc atcgactcac |
| 961 | atctgcggga taagctcccc aacatttcct gtatcaaggc cattgatatc tatatcctcg |
| 1021 | tgtgcttgtt ctttgtgttc ctgtccttgc tggagtatgt ctacatcaac tatcttttct |
| 1081 | acagtcgagg acctcggcgc cagcctaggc gacacaggag accccgaaga gtcattgccc |
| 1141 | gctaccgcta ccagcaagtg gtggtaggaa acgtgcagga tggcctgatt aacgtggaag |
| 1201 | acggagtcag ctctctcccc atcaccccag cgcaggcccc cctggcaagc ccggaaagcc |
| 1261 | tcggttcttt gacgtccacc tccgagcagg cccagctggc cacctcggaa agcctcagcc |
| 1321 | cactcacttc tctctcaggc caggcccccc tggccactgg agaaagcctg agcgatctcc |
| 1381 | cctccacctc agagcaggcc cggcacagct atggtgttcg ctttaatggt ttccaggctg |
| 1441 | atgacagtat tattcctacc gaaatccgca accgtgtcga agcccatggc catggtgtta |
| 1501 | cccatgacca tgaagattcc aatgagagct tgagctcgga tgagcgccat ggccatggcc |
| 1561 | ccagtgggaa gcccatgctt caccatggcg agaagggtgt gcaagaagca ggctgggacc |
| 1621 | ttgatgacaa caatgacaag agcgactgcc ttgccattaa ggagcaattc aagtgtgata |
| 1681 | ctaacagtac ctggggcctt aatgatgatg agctcatggc ccatggccaa gagaaggaca |
| 1741 | gtagctcaga gtctgaggat agttgccccc caagccctgg gtgctccttc actgaagggt |
| 1801 | tctccttcga tctctttaat cctgactacg tcccaaaggt cgacaagtgg tcccggttcc |
| 1861 | tcttccctct ggcctttggg ttgttcaaca ttgtttactg ggtataccat atgtattagt |
| 1921 | cccccagtgc tccagaacag cgggagcact gtgctgtgct cctttcagtt tcttttgggt |
| 1981 | ttgtttttcc ctctttcctt |

An exemplary human GABRR1 amino acid sequence is set forth below (SEQ ID NO: 35; GenBank Accession No: EAW48558.1, Version 1, incorporated herein by reference):

| | |
|---|---|
| 1 | mlavpnmrfg ifllwwgwvl atesrmhwpg revhemskkg rpqrqrrevh edahkqvspi |
| 61 | lrrspditks pltkseqllr iddhdfsmrp gfggpaipvg vdvqveslds isevdmdftm |
| 121 | tlylrhywkd erlsfpstnn lsmtfdgrlv kkiwvpdmff vhskrsfihd tttdnvmlrv |
| 181 | qpdgkvlysl rvtvtamcnm dfsrfpldtq tcsleiesya yteddlmlyw kkgndslktd |
| 241 | erislsqfli qefhtttkla fysstgwynr lyinftlrrh ifffllqtyf patlmvmlsw |
| 301 | vsfwidrrav parvplgitt vltmstiitg vnasmprvsy ikavdiylwv sfvfvflsvl |
| 361 | eyaavnyltt vqerkeqklr eklpctsglp pprtamldgn ysdgevndld nympengekp |
| 421 | drmmvqltla sersspqrks qrssyvsmri dthaidkysr iifpaayilf nliywsifs |

An exemplary human GABRR1 nucleic acid sequence is set forth below (SEQ ID NO: 36; GenBank Accession No: NM_002042.4, Version 4, incorporated herein by reference):

| | |
|---|---|
| 1 | taataatggc cgtaagctta aaatagatcc agggaggagc tcattaacgt gaacatagaa |
| 61 | agcagttccg cacctctggc cttactcctc ttggaaattg ctttggtcca tttttacttc |
| 121 | cttttattcg acgcaccaga aaataagact tttaccaaca tttttactgc atttgacgat |
| 181 | gaactaattt agaccggcta aaataattgt tccactggga cacaggaatt caacctcagt |
| 241 | tcagaaaatc cctgacatct gacgtaggag gatttatagg tttagtggaa attgctttct |
| 301 | cctgctctcc agattgcatc ctgtgggttg attttttttt tgcatgagta aacatccttc |
| 361 | taataatgaa cagaccaata atgtcttaag agagaaaaag aacaatcttt tcctttttgc |
| 421 | tgtttctgga gagagctgtt tgaatttgga aacccatgtt ggctgtccca aatatgagat |
| 481 | ttggcatctt tcttttgtgg tggggatggg ttttggccac tgaaagcaga atgcactggc |
| 541 | ccggaagaga agtccacgag atgtctaaga aaggcaggcc ccaaagacaa agacgagaag |
| 601 | tacatgaaga tgcccacaag caagtcagcc caattctgag acgaagtcct gacatcacca |
| 661 | aatcgcctct gacaaagtca gaacagcttc tgaggataga tgaccatgat ttcagcatga |
| 721 | ggcctggctt tggaggccct gccattcctg ttggtgtgga tgtgcaggtg gagagtttgg |
| 781 | atagcatctc agaggttgac atggacttta cgatgaccct ctacctgagg cactactgga |
| 841 | aggacgagag gctgtctttt ccaagcacca acaacctcag catgacgttt gacggccggc |
| 901 | tggtcaagaa gatctgggtc cctgacatgt ttttcgtgca ctccaaacgc tccttcatcc |
| 961 | acgacaccac cacagacaac gtcatgttgc gggtccagcc tgatgggaaa gtgctctata |
| 1021 | gtctcagggt tacagtaact gcaatgtgca acatggactt cagccgattt cccttggaca |
| 1081 | cacaaacgtg ctctcttgaa attgaaagct atgcctatac agaagatgac ctcatgctgt |
| 1141 | actggaaaaa gggcaatgac tccttaaaga cagatgaacg gatctcactc tcccagttcc |
| 1201 | tcattcagga attccacacc accaccaaac tggctttcta cagcagcaca ggctggtaca |
| 1261 | accgtctcta cattaatttc acgttgcgtc gccacatctt cttcttcttg ctccaaactt |
| 1321 | atttccccgc taccctgatg gtcatgctgt cctgggtgtc cttctggatc gaccgcagag |
| 1381 | ccgtgcctgc cagagtcccc ttaggtatca caacggtgct gaccatgtcc accatcatca |
| 1441 | cgggcgtgaa tgcctccatg ccgcgcgtct cctacatcaa ggccgtggac atctacctct |
| 1501 | gggtcagctt tgtgttcgtg ttcctctcgg tgctggagta tgcggccgtc aactacctga |
| 1561 | ccactgtgca ggagaggaag gaacagaagc tgcgggagaa gcttccctgc accagcggat |
| 1621 | tacctccgcc ccgcactgcg atgctggacg gcaactacag tgatggggag gtgaatgacc |
| 1681 | tggacaacta catgccagag aatggagaga agcccgacag gatgatggtg cagctgaccc |
| 1741 | tggcctcaga gaggagctcc ccacagagga aaagtcagag aagcagctat gtgagcatga |
| 1801 | gaatcgacac ccacgccatt gataaatact ccaggatcat ctttccagca gcatacattt |
| 1861 | tattcaattt aatatactgg tctattttct cctagatgct tgtaattcta caaatttcac |
| 1921 | atttccatgg catgcactac agaaataact gtataatgaa aaagtattta aggatatggt |
| 1981 | taaaaaaaaa tcccaggacc cacccatgtt ttcactatcc cttctgcagc tttccaaagc |
| 2041 | tacattgacg agacacttac tggtttaatt tgcacttatt aaccatctat tgaatacaca |
| 2101 | gcattatatt aggtgctgca ggaaatacga cactgtagcg actgatgtta gttgttaccc |
| 2161 | agatcccctg gaaaagcaca ctaccagtgt tgtgggcaca tttagttcca cccgttagac |
| 2221 | ccttgatgct attcacatga ataatttatt ttcctcaagt gtcattacat tgttcaggct |
| 2281 | acgtgaactt ggaagcacct acaggccatt tgcatgaaat tcacatgcac ctaaatcctc |
| 2341 | actttgacag aaactcatgc ttcagtttat aacctattac ctattttgta tgcgactcca |
| 2401 | cctccgcatg tttattttaa taaaaggcaa tgataacatt cacattattt ttctttatat |
| 2461 | gctgtggttc acaggcttta ccccttcaca agaaaagctc tttagattgg cgcaattgct |
| 2521 | tctgattttg gtgaaatttt ccctggtagg gaaactttga agataagagt acacacatgc |
| 2581 | attttgtctg ttgtgtcata gaggtaacta ggctagaaaa tttgtgttta aatgttccct |
| 2641 | attttatata atcaccactt catgtttctt cttcttggag catgtccttg ttcaaagaga |
| 2701 | agtgctttct cagtgatgtg atatcttcac tgaggaactt gggtagagaa tgatttcttc |
| 2761 | tgcataaaca cttcaaggaa atacataatt tgggactact tgtaactcat tagaatgaga |
| 2821 | aatactcaca tggtttctta agagaaaaag aacatcggaa agcaaaataa atgggaagat |
| 2881 | atcactggac atctgcattt atactcgaaa taccagcatt ttctatggac cagaaaactg |
| 2941 | ccatcaccta gaccacacag cccagatacc aggcagacgg atggcccaat ggcaactgat |
| 3001 | gtcagggcat ggggtaaagg agagggttct aatctggtgt atcacttaaa aacagttatt |
| 3061 | tatattatat atctgctata tagatcaacc tccaccaaac ttacccaaac agcatttgtt |
| 3121 | ttatttgaaa ctcactttaa taaagtgaat tatatacaca aaaaaaaaaa aaaa |

An exemplary human CLDN1 amino acid sequence is set forth below (SEQ ID NO: 37; GenBank Accession No: CAG33419.1, Version 1, incorporated herein by reference):

| | |
|---|---|
| 1 | managlqllg filaflgwig aivstalpqw riysyagdni vtaqamyegl wmscvsqstg |
| 61 | qiqckvfdsl lnlsstlqat ralmvvgill gviaifvatv gmkcmkcled devqkmrmav |
| 121 | iggaifllag lailvatawy gnrivqefyd pmtpvnarye fgqalftgwa aaslcllgga |
| 181 | llccscprkt tsyptprpyp kpapssgkdy v |

An exemplary human CLDN1 nucleic acid sequence is set forth below (SEQ ID NO: 38; GenBank Accession No: NM_021101.4, Version 4, incorporated herein by reference):

| | |
|---|---|
| 1 | gtctcagttc ccgagcctgg gagcaaccgc agcttctagt atccagactc cagcgccgcc |
| 61 | ccgggcgcgg accccaaccc cgacccagag cttctccagc ggcggcgcag cgagcagggc |
| 121 | tccccgcctt aacttcctcc gcggggccca gccaccttcg ggagtccggg ttgcccacct |
| 181 | gcaaactctc cgccttctgc acctgccacc cctgagccag cgcgggcgcc cgagcgagtc |
| 241 | atggccaacg cggggctgca gctgttgggc ttcattctcg ccttcctggg atggatcggc |
| 301 | gccatcgtca gcactgccct gccccagtgg aggatttact cctatgccgg cgacaacatc |
| 361 | gtgaccgccc aggccatgta cgaggggctg tggatgtcct gcgtgtcgca gagcaccggg |
| 421 | cagatccagt gcaaagtctt tgactccttg ctgaatctga gcagcacatt gcaagcaacc |
| 481 | cgtgccttga tggtggttgg catcctcctg ggagtgatag caatctttgt ggccaccgtt |
| 541 | ggcatgaagt gtatgaagtg cttggaagac gatgaggtgc agaagatgag gatggctgtc |
| 601 | attgggggtg cgatatttct tcttgcaggt ctggctattt tagttgccac agcatggtat |
| 661 | ggcaatagaa tcgttcaaga attctatgac cctatgaccc cagtcaatgc caggtacgaa |
| 721 | tttggtcagg ctctcttcac tggctgggct gctgcttctc tctgccttct gggaggtgcc |
| 781 | ctactttgct gttcctgtcc ccgaaaaaca acctcttacc caacaccaag gccctatcca |
| 841 | aaacctgcac cttccagcgg gaaagactac gtgtgacaca gaggcaaaag gagaaaatca |
| 901 | tgttgaaaca aaccgaaaat ggacattgag atactatcat taacattagg accttagaat |
| 961 | tttgggtatt gtaatctgaa gtatggtatt acaaaacaaa caaacaaaca aaaaacccat |
| 1021 | gtgttaaaat actcagtgct aaacatggct taatcttatt ttatcttctt tcctcaatat |
| 1081 | aggagggaag atttttccat ttgtattact gcttcccatt gagtaatcat actcaactgg |
| 1141 | gggaaggggt gctccttaaa tatatataga tatgtatata tacatgtttt tctattaaaa |
| 1201 | atagacagta aaatactatt ctcattatgt tgatactagc atacttaaaa tatctctaaa |
| 1261 | ataggtaaat gtatttaatt ccatattgat gaagatgttt attggtatat tttctttttc |
| 1321 | gtctatatat acatatgtaa cagtcaaata tcatttactc ttcttcatta gctttgggtg |
| 1381 | cctttgccac aagacctagc ctaatttacc aaggatgaat tctttcaatt cttcatgcgt |
| 1441 | gcccttttca tatacttatt ttatttttta ccataatctt atagcacttg catcgttatt |
| 1501 | aagcccttat ttgttttgtg tttcattggt ctctatctcc tgaatctaac acatttcata |
| 1561 | gcctacattt tagtttctaa agccaagaag aatttattac aaatcagaac tttggaggca |
| 1621 | aatctttctg catgaccaaa gtgataaatt cctgttgacc ttcccacaca atccctgtac |
| 1681 | tctgacccat agcactcttg tttgctttga aaatatttgt ccaattgagt agctgcatgc |
| 1741 | tgttccccca ggtgttgtaa cacaacttta ttgattgaat ttttaagcta cttattcata |
| 1801 | gttttatatc cccctaaact acctttttgt tccccattcc ttaattgtat tgttttccca |
| 1861 | agtgtaatta tcatgcgttt tatatcttcc taataaggtg tggtctgttt gtctgaacaa |
| 1921 | agtgctagac tttctggagt gataatctgg tgacaaatat tctctctgta gctgtaagca |
| 1981 | agtcacttaa tctttctacc tcttttttct atctgccaaa ttgagataat gatacttaac |
| 2041 | cagttagaag aggtagtgtg aatattaatt agtttatatt actctcattc tttgaacatg |
| 2101 | aactatgcct atgtagtgtc tttatttgct cagctggctg agacactgaa gaagtcactg |
| 2161 | aacaaaacct acacacgtac cttcatgtga ttcactgcct tcctctctct accagtctat |
| 2221 | ttccactgaa caaaacctac acacatacct tcatgtggtt cagtgccttc ctctctctac |
| 2281 | cagtctattt ccactgaaca aaacctacgc acataccttc atgtggctca gtgccttcct |
| 2341 | ctctctacca gtctatttcc attctttcag ctgtgtctga catgtttgtg ctctgttcca |
| 2401 | ttttaacaac tgctcttact tttccagtct gtacagaatg ctatttcact tgagcaagat |
| 2461 | gatgtaatgg aaagggtgtt ggcattggtg tctggagacc tggatttgag tcttggtgct |
| 2521 | atcaatcacc gtctgtgttt gagcaaggca tttggctgct gtaagcttat tgcttcatct |
| 2581 | gtaagcggtg gtttgtaatt cctgatcttc ccacctcaca gtgatgttgt ggggatccag |
| 2641 | tgagatagaa tacatgtaag tgtggttttg taatttaaaa agtgctatac taagggaaag |
| 2701 | aattgaggaa ttaactgcat acgttttggt gttgcttttc aaatgtttga aaacaaaaaa |
| 2761 | aatgttaaga aatgggtttc ttgccttaac cagtctctca agtgatgaga cagtgaagta |
| 2821 | aaattgagtg cactaaacaa ataagattct gaggaagtct tatcttctgc agtgagtatg |
| 2881 | gcccgatgct ttctgtggct aaacagatgt aatgggaaga aataaaagcc tacgtgttgg |
| 2941 | taaatccaac agcaagggag atttttgaat cataataact cataaggtgc tatctgttca |
| 3001 | gtgatgccct cagagctctt gctgttagct ggcagctgac gctgctagga tagttagttt |
| 3061 | ggaaatggta cttcataata aactacacaa ggaaagtcag ccactgtgtc ttatgaggaa |
| 3121 | ttggacctaa taaattttag tgtgccttcc aaacctgaga atatatgctt ttggaagtta |
| 3181 | aaatttaaat ggcttttgcc acatacatag atcttcatga tgtgtgagtg taattccatg |
| 3241 | tggatatcag ttaccaaaca ttacaaaaaa attttatggc ccaaaatgac caacgaaatt |
| 3301 | gttacaatag aatttatcca attttgatct ttttatattc ttctaccaca cctggaaaca |
| 3361 | gaccaataga cattttgggg ttttataata ggaatttgta taaagcatta ctctttttca |
| 3421 | ataaattgtt ttttaattta aaaaaaggat ta |

An exemplary human CLDN2 amino acid sequence is set forth below (SEQ ID NO: 39; GenBank Accession No: AAH71747.1, Version 1, incorporated herein by reference):

| | |
|---|---|
| 1 | maslglqlvg yilgllgllg tlvamllpsw ktssyvgasi vtavgfskgl wmecathstg |
| 61 | itqcdiystl lglpadigaa qammvtssai sslaciisvv gmrctvfcge srakdrvava |
| 121 | ggvffilggl lgfipvawnl hgilrdfysp lvpdsmkfei gealylgiis slfsliagii |
| 181 | lcfscssqrn rsnyydayqa qplatrsspr pgqppkvkse fnsysltgyv |

An exemplary human CLDN2 nucleic acid sequence is set forth below (SEQ ID NO: 40; GenBank Accession No: NM_020384.3, Version 3, incorporated herein by reference):

| | |
|---|---|
| 1 | aagtctctga cccacggctc agatttgcag atggattttg caaagctgtg gttaacgatt |
| 61 | agaaatcctt tatcacctca gcccgtggcc ccttgtactt cgctcccctc cctcaggatc |
| 121 | cctttctccc tctccagggg catctccccc tccaaggctc tgcaaagaac tgccctgtct |
| 181 | tctagatgcc ttcttgaggc tgcttgtggc cacccacaga cacttgtaag gaggagagaa |
| 241 | gtcagcctgg cagagagact ctgaaatgag ggattagagg tgttcaagga gcaagagctt |
| 301 | cagcctgaag acaagggagc agtccctgaa gacgcttcta ctgagaggtc tgccatggcc |
| 361 | tctcttggcc tccaacttgt gggctacatc ctaggccttc tggggctttt gggcacactg |
| 421 | gttgccatgc tgctccccag ctggaaaaca agttcttatg tcggtgccag cattgtgaca |
| 481 | gcagttggct tctccaaggg cctctggatg gaatgtgcca cacacagcac aggcatcacc |
| 541 | cagtgtgaca tctatagcac ccttctgggc ctgcccgctg acatccaggc tgcccaggcc |
| 601 | atgatggtga catccagtgc aatctcctcc ctggcctgca ttatctctgt ggtgggcatg |
| 661 | agatgcacag tcttctgcca ggaatcccga gccaaagaca gagtggcggt agcaggtgga |
| 721 | gtctttttca tccttggagg cctcctggga ttcattcctg ttgcctggaa tcttcatggg |
| 781 | atcctacggg acttctactc accactggtg cctgacagca tgaaatttga gattggagag |
| 841 | gctctttact tgggcattat ttcttccctg ttctccctga tagctggaat catcctctgc |
| 901 | ttttcctgct catcccagag aaatcgctcc aactactacg atgcctacca agcccaacct |
| 961 | cttgccacaa ggagctctcc aaggcctggt caacctccca aagtcaagag tgagttcaat |
| 1021 | tcctacagcc tgacagggta tgtgtgaaga accaggggcc agagctgggg ggtggctggg |
| 1081 | tctgtgaaaa acagtggaca gcaccccgag ggccacaggt gagggacact accactggat |
| 1141 | cgtgtcagaa ggtgctgctg aggatagact gactttggcc attggattga gcaaaggcag |
| 1201 | aaatgggggc tagtgtaaca gcatgcaggt tgaattgcca aggatgctcg ccatgccagc |
| 1261 | ctttctgttt tcctcacctt gctgctcccc tgccctaagt ccccaaccct caacttgaaa |
| 1321 | ccccattccc ttaagccagg actcagagga tccctttgcc ctctggttta cctgggactc |
| 1381 | catccccaaa cccactaatc acatcccact gactgaccct ctgtgatcaa agaccctctc |
| 1441 | tctggctgag gttggctctt agctcattgc tggggatggg aaggagaagc agtggctttt |
| 1501 | gtgggcattg ctctaaccta cttctcaagc ttccctccaa agaaactgat tggccctgga |
| 1561 | acctccatcc cactcttgtt atgactccac agtgtccaga ctaatttgtg catgaactga |
| 1621 | aataaaacca tcctacggta tccagggaac agaaagcagg atgcaggatg ggaggacagg |
| 1681 | aaggcagcct gggacattta aaaaaataaa aatgaaaaaa aaacccagaa cccatttctc |
| 1741 | agggcacttt ccagaattct ctcatatttg tgggctggga tcaagcctgc agcttgagga |
| 1801 | aagcacaagg aaaggaaaga agatctggtg gaaagctcag gtggcagcgg actctgactc |
| 1861 | cactgaggaa ctgcctcaga agctgcgatc acaactttgg ctgaagcccc tgcctcactc |
| 1921 | tagggcacct gacctggcct cttgcctaaa ccacaaggct aagggctata gacaatggtt |
| 1981 | tccttaggaa cagtaaacca gtttttctag ggatggccct tggctggggg atgacagtgt |
| 2041 | gggagctgtg gggtactgag gaagacacca ttccttgacg gtgtctaaga agccaggtgg |
| 2101 | atgtgtgtgg tggctccagt gggtgtttct actctgccag tgagaggcag ccccctagaa |
| 2161 | actcttcagg cgtaatggaa aatcagctca aatgagatca ggccccccca gggtccaccc |
| 2221 | acagagcact acagagcctc tgaaagacca tagcaccaag cgagcccctt cagattcccc |
| 2281 | cactgtccat cggaagatgc tccagagtgg ctagagggca tctaagggct ccagcatggc |
| 2341 | atatccatgc ccacggtgct gtgtccatga tctgagtgat agctgcactg ctgcctggga |
| 2401 | ttgcagctga ggtgggagtg gagaatggtt cccaggaaga cagttccacc tctaaggtcc |
| 2461 | gaaaatgttc cctttaccct ggagtgggag tgaggggtca tacaccaaag gtattttccc |
| 2521 | tcaccagtct aggcatgact ggcttctgaa aaattccagc acacctcctc gaacctcatt |
| 2581 | gtcagcagag agggcccatc tgttgtctgt aacatgcctt tcacatgtcc accttcttgc |
| 2641 | catgttccag ctgctctccc aacctggaag gccgtctccc cttagccaag tcctcctcag |
| 2701 | gcttggagaa cttcctcagc gtcacctcct tcattgagcc ttctctgatc actccatccc |
| 2761 | tctcctaccc ctccctcccc caaccctcaa tgtataaatt gcttcttgat gcttagcatt |
| 2821 | cacaattttt gattgatcgt tatttgtgtg tgtgtgtccg atctcacaag tatattgtaa |
| 2881 | acccttcggt gggtgggggc catatcctag acctctctgt atcccccaga ctatctgtaa |
| 2941 | cagtgccagg cacacagtag gtgatcaata aacacttgtt gattgagaaa aaaaaaaa |

An exemplary human EYA1 amino acid sequence is set forth below (SEQ ID NO: 41; GenBank Accession No: AAI21799.1, Version 1, incorporated herein by reference):

| | |
|---|---|
| 1 | mfpsnawafy flsfltnsrp yphilptpss qtmaaygqtq fttgmqqata yatypqpgqp |
| 61 | ygissygalw agikteggls qsqspgqtgf lsygtsfstp qpgqapysyq mqgssfttss |
| 121 | giytgnnslt nssgfnssqq dypsypsfgq gqyaqyynss pypahymtss ntspttpstn |
| 181 | atyqlqepps gitsqavtdp taeystihsp stpikdsdsd rlrrgsdgks rgrgrrnnnp |
| 241 | spppdsdler vfiwdldeti ivfhslltgs yanrygrdpp tsyslglrme emifnladth |
| 301 | lffndleecd qvhiddvssd dngqdlstyn fgtdgfpaaa tsanlclatg vrggvdwmrk |
| 361 | lafryrrvke iyntyknnvg gllgpakrea wlqlraeiea ltdswltlal kalslihsrt |
| 421 | ncvnilvttt qlipalakvl lyglgivfpi eniysatkig kescferiiq rfgrkvvyvv |
| 481 | igdgveeeqg akkhampfwr isshsdlmal hhaleleyl |

An exemplary human EYA1 nucleic acid sequence is set forth below (SEQ ID NO: 42; GenBank Accession No: NM_001288574.1, Version 1, incorporated herein by reference):

| | |
|---|---|
| 1 | aaaccaataa ggttaggaca agagaatagc tgtggtttgc gttgcaaaaa ccaaaaaaaa |
| 61 | aaaaaaaaaa aaaaaaagaa agccccgagg ctccatgggc agacctacaa ggctgcgcaa |
| 121 | acaaatcgag ggatgagatt ctgctgtttc tttgtctagg gttctcagat gctatctgcc |
| 181 | gctgctgttt ggtggggaag gagcgctggg cgcaaagctg ttaccaaaca gaacggtggg |
| 241 | agctgatggc tccgagtttg gggcgaggta gaaactctcc agtgccactt ccgactttaa |
| 301 | gccttcctgt tgccgtccac tgtggcgggt ttcttcctgg ggaacacgtt ttcgctcagt |
| 361 | cgctcggcag cccgagcctg cggcagcggc caggcgcctg ccccctgcgc cgagctttcc |
| 421 | cctgcagagg cgctccactc ccagaagcgc cgcggctgca ccagagcgcc tgagagcccc |
| 481 | cgcgcgtacc catccaggag caaaactatg tcaggaatgg aggtttgcta acccagaaaa |
| 541 | ttcgaaggaa cacattaaac tggtggatgc agcagatgta agcgctgtgc aaacatctca |
| 601 | agccagttca gatgttgctg tttcctcaag ttgcaggtct atggaaatgc aggatctaac |
| 661 | cagcccgcat agccgtctga gtggtagtag tgaatccccc agtggcccca aactcggtaa |
| 721 | ctctcatata aatagtaatt ccatgactcc caatggcacc gaagttaaaa cagagccaat |
| 781 | gagcagcagt gaaacagctt caacgacagc cgacgggtct ttaaacaatt tctcaggttc |
| 841 | agcaattggg agcagtagtt tcagcccacg accaactcac cagttctctc caccacagat |
| 901 | ttacccttcc aaaccatacc cacatattct ccctacccct tcctcacaaa ctatggctgc |
| 961 | atatgggcaa acacagttta ccacaggaat gcaacaagct acagcctatg ccacgtaccc |
| 1021 | acagccagga cagccgtacg gcatttcctc atatggcatc aagactgaag gtggattgtc |
| 1081 | acagtctcag tcacctggac agacaggatt tctcagctat ggcacaagct tcagtacccc |
| 1141 | tcaacctgga caggcaccat acagctacca gatgcaaggt agcagtttta caacatcatc |
| 1201 | aggaatatat acaggaaata attcactcac aaattcctct ggatttaata gttcacagca |
| 1261 | ggactatccg tcttatccca gttttggcca gggtcagtac gcacagtatt ataacagctc |
| 1321 | accgtatcca gcacattata tgaccagcag caacaccagc ccaacgacac catccaccaa |
| 1381 | tgccacttac cagcttcaag aaccgccatc tggcatcacc agccaagcag ttacagatcc |
| 1441 | cacagcagag tacagcacaa tccacagccc atcaacaccc attaaagatt cagattctga |
| 1501 | tcgattgcgt cgaggttcag atgggaaatc acgtggacgg ggccgaagaa acaataatcc |
| 1561 | ttcacctccc ccagattctg atcttgagag agtgttcatc tgggacttgg atgagacaat |
| 1621 | cattgttttc cactccttgc ttactgggtc ctacgccaac agatatggga gggatccacc |
| 1681 | cacttcagtt tcccttggac tgcgaatgga agaaatgatt ttcaacttgg cagacacaca |
| 1741 | tttatttttt aatgacttag aagaatgtga ccaagtccat atagatgatg tttcttcaga |
| 1801 | tgataacgga caggacctaa gcacatataa ctttggaaca gatggctttc ctgctgcagc |
| 1861 | aaccagtgct aacttatgtt tggcaactgg tgtacggggc ggtgtggact ggatgagaaa |
| 1921 | gttggccttc cgctacagac gggtaaaaga gatctacaac acctacaaaa ataatgttgg |
| 1981 | aggtctgctt ggtccagcta agagggaagc ctggctgcag ttgagggccg aaattgaagc |
| 2041 | cctgaccgac tcctggttga cactggccct gaaagcactc tcgctcattc actcccggac |
| 2101 | aaactgtgtg aatattttag taacaactac tcagctcatc ccagcattgg cgaaagtcct |
| 2161 | gctgtatggg ttaggaattg tatttccaat agaaaatatt tacagtgcaa ctaaaatagg |
| 2221 | aaaagaaagc tgttttgaga gaataattca aaggtttgga agaaaagtgg tgtatgttgt |
| 2281 | tataggagat ggtgtagaag aagaacaagg agcaaaaaag cacgcgatgc ccttctggag |
| 2341 | gatctccagc cactcggacc tcatggccct gcaccatgcc ttggaactgg agtacctgta |
| 2401 | acagcgctcg gcactttgac agcgcacagc tgctctgtga ccagggacag atccagcagg |
| 2461 | ccccagtctc gcatcagcgc cggcctccag aacttagcaa tttccgcctg gtgatgcgca |
| 2521 | gttgctgtca gtcttgacct ctgcctttgt ggtgaatgga ggaccacgtc tatttcatca |
| 2581 | gaacagctgt tgactctagt actgtgaatc cagtgaaaat aagccatgag aatgttttag |
| 2641 | cacagcgtta tgtgtctgcc acattaacta cacggttcaa acctgtgaag aaaggacctg |
| 2701 | caaacgcttc agttgttagc attttcaatg tgatataaac agcttctcca atacagcaaa |
| 2761 | cctaattgca caacagagac tgaaatgtgt ttcctgaata ccagtggagg aattttcttg |
| 2821 | taaagaaggt ttactttttg gtgtctcata cccagggtaa tctgtacatc tctacttatt |
| 2881 | tatgaacaga ctttttttaa aaagataaaa aaacagcttt attgaggtat aattcaccca |
| 2941 | ccagactttt ttaaacatca aataattgaa gagacaatag cattagaaat aagtgattaa |
| 3001 | aggcctctgc ctcacaacat ggcaagtaca gtactttgaa ttttagcaca ttgcatagta |
| 3061 | gttttaagta tgtctaattt aaacgtataa tatgtacatc actgagacaa tcatgtacag |
| 3121 | aaagaatttt tggtgtaaat ttgtaataat ggataattct tttacatatt gtttagggaa |
| 3181 | atgatattga aaggtagcaa tgcctggata gtgaagcatg aggcagcacg tgcacaaatt |
| 3241 | catgtgccgt gccttatctg agttttcggt ataaatatgt agataatgga tttttttttt |
| 3301 | ttagataatg ttgtcaagac caaaagcatg gatgtcaagt gtcagtaagg attttgtttt |
| 3361 | ctaaaatttt ttcctgcatc agttcttctg agggccttga tgaaataaca cagcagtttc |
| 3421 | ttaaacaatt tgaaacaaaa tgagctctcc taccacctca ctttttcatt tccacactaa |
| 3481 | tgtattatat gtaactactt ggaaaaaata attattcaaa tgcttcttcc cacaaagaat |
| 3541 | atagatgata gtagatatat tttattaata aaatggttca tgaatcggag actaacaaag |
| 3601 | ttttcatgtg ctcagaatta ttaattatcg tgtctgcatt ttctttcgat aaaggaagac |
| 3661 | acacgatgct aatccggaaa tcagcaaact ttgcattact ccctatgtgc gtattttctc |
| 3721 | tttcttcctg tcaccctgag gaaggttcat tgccattgtc atcaccatgg aaacaacgtt |
| 3781 | cctctccacc tgcattatgt actacatgac aggcatcaat ctggggaaat aataaaatta |
| 3841 | tcacctttgt cagaccataa gagtttctcc aaaagtggtc agtttggctg ggcaatattt |
| 3901 | tctctcatct aacaaacaca atccattgtc atgaaattac ccttaggatg agtcttcttt |
| 3961 | aatcaatcat atattgggcg gaaaaaacac cagctttgac ccgaagtagt tgaagagcta |
| 4021 | cttcattctt ttctgaagtt gtgtgttgct gctagaaata gtcatttgtg aattatccaa |
| 4081 | attgtttaaa ttcacaattg aattagtttt ttcttccttt ttgcttgaag caaacagttg |
| 4141 | acaattttta accttttcat tttatgtttt tgtactctgc agactgaaaa gacaaagttt |
| 4201 | atcttggcct tactgtataa aggtgtgctg tgtccaccgt tgtgtacaga atttttcttc |
| 4261 | attaattttg tgtttaagtt aataaaattt atttgtgatg tactgtaaaa aaaaaaaaaa |
| 4321 | aaa |

An exemplary human SNAI1 amino acid sequence is set forth below (SEQ ID NO: 43; GenBank Accession No: CAB52414.1, Version 1, incorporated herein by reference):

| | |
|---|---|
| 1 | mprsflvrkp sdpnrkpnys elqdsnpeft fqqpydqahl laaipppeil nptaslpmli |
| 61 | wdsvlapqaq piawaslrlq esprvaelts lsdedsgkgs qppsppspap ssfsstsvss |
| 121 | leaeayaafp glgqvpkqla qlseakdlqa rkafnckycn keylslgalk mhirshtlpc |
| 181 | vcgtcgkafs rpwllqghvr thtgekpfsc phcsrafadr snlrahlqth sdvkkyqcqa |
| 241 | cartfsrmsl lhkhqesgcs gcpr |

An exemplary human SNAI1 nucleic acid sequence is set forth below (SEQ ID NO: 44; GenBank Accession No: NM_005985.3, Version 3, incorporated herein by reference):

| | |
|---|---|
| 1 | attcattgcg ccgcggcacg gcctagcgag tggttcttct gcgctactgc tgcgcgaatc |
| 61 | ggcgacccca gtgcctcgac cactatgccg cgctctttcc tcgtcaggaa gccctccgac |
| 121 | cccaatcgga agcctaacta cagcgagctg caggactcta atccagagtt taccttccag |
| 181 | cagccctacg accaggccca cctgctggca gccatcccac ctccggagat cctcaacccc |
| 241 | accgcctcgc tgccaatgct catctgggac tctgtcctgg cgccccaagc ccagccaatt |
| 301 | gcctgggcct cccttcggct ccaggagagt cccagggtgg cagagctgac ctccctgtca |
| 361 | gatgaggaca gtgggaaagg ctcccagccc cccagcccac cctcaccggc tccttcgtcc |
| 421 | ttctcctcta cttcagtctc ttccttggag gccgaggcct atgctgcctt cccaggcttg |
| 481 | ggccaagtgc ccaagcagct ggcccagctc tctgaggcca aggatctcca ggctcgaaag |
| 541 | gccttcaact gcaaatactg caacaaggaa tacctcagcc tgggtgccct caagatgcac |
| 601 | atccgaagcc acacgctgcc ctgcgtctgc ggaacctgcg ggaaggcctt ctctaggccc |
| 661 | tggctgctac aaggccatgt ccggacccac actggcgaga agcccttctc ctgtccccac |
| 721 | tgcagccgtg ccttcgctga ccgctccaac ctgcgggccc acctccagac ccactcagat |
| 781 | gtcaagaagt accagtgcca ggcgtgtgct cggaccttct cccgaatgtc cctgctccac |
| 841 | aagcaccaag agtccggctg ctcaggatgt ccccgctgac cctcgaggct ccctcttcct |
| 901 | ctccatacct gcccctgcct gacagccttc cccagctcca gcaggaagga ccccacatcc |
| 961 | ttctcactgc catggaattc cctcctgagt gccccacttc tggccacatc agccccacag |
| 1021 | gactttgatg aagaccattt tctggttctg tgtcctctgc ctgggctctg gaagaggcct |
| 1081 | tcccatggcc atttctgtgg agggagggca gctggccccc agccctgggg gattcctgag |
| 1141 | ctggcctgtc tgcgtgggtt tttgtatcca gagctgtttg gatacagctg ctttgagcta |
| 1201 | caggacaaag gctgacagac tcactgggaa gctcccaccc cactcagggg accccactcc |
| 1261 | cctcacacac acccccccac aaggaaccct caggccaccc tccacgaggt gtgactaact |
| 1321 | atgcaataat ccacccccag gtgcagcccc agggcctgcg gaggcggtgg cagactagag |
| 1381 | tctgagatgc cccgagccca ggcagctatt tcagcctcct gtttggtggg gtggcacctg |
| 1441 | tttcccgggc aatttaacaa tgtctgaaaa gggactgtga gtaatggctg tcacttgtcg |
| 1501 | ggggcccaag tggggtgctc tggtctgacc gatgtgtctc ccagaactat tctgggggcc |
| 1561 | cgacaggtgg gcctgggagg aagatgttta catttttaaa ggtacactgg tatttatatt |
| 1621 | tcaaacattt tgtatcaagg aaacgttttg tatagttata tgtacagttt attgatattc |
| 1681 | aataaagcag ttaatttata tattaaaaaa aaaaaaaaaa aa |

An exemplary human TGFB2 amino acid sequence is set forth below (SEQ ID NO: 45; GenBank Accession No: AAH99635.1, Version 1, incorporated herein by reference):

| | |
|---|---|
| 1 | mhycvlsafl ilhlvtvals lstcstldmd qfmrkrieai rgqilsklkl tsppedypep |
| 61 | eevppevisi ynstrdllqe kasrraaace rersdeeyya kevykidmpp ffpsenaipp |
| 121 | tfyrpyfriv rfdvsamekn asnlvkaefr vfrlqnpkar vpeqrielyq ilkskdltsp |
| 181 | tqryidskvv ktraegewls fdvtdavhew lhhkdrnlgf kislhcpcct fvpsnnyiip |
| 241 | nkseelearf agidgtstyt sgdqktikst rkknsgktph lllmllpsyr lesqqtnrrk |
| 301 | kraldaaycf rnvgdncclr plyidfkrdl gwkwihepkg ynanfcagac pylwssdtqh |
| 361 | srvlslynti npeasaspcc vsqdleplti lyyigktpki eqlsnmivks ckcs |

An exemplary human TGFB2 nucleic acid sequence is set forth below (SEQ ID NO: 46; GenBank Accession No: NM_001135599.3, Version 3, incorporated herein by reference):

| | |
|---|---|
| 1 | agacacgtgg ttcagagaga acttataaat ctcccctccc cggcaagatc gtgatgttat |
| 61 | ctgctggcag cagaaggttc gctccgagcg gagctccaga agctcctgac aagagaaaga |
| 121 | cagattgaga tagagataga aagagaaaga gagaaagaga cagcagagcg agagcgcaag |
| 181 | tgaaagaggc aggggagggg gatggagaat attagcctga cggtctaggg agtcatccag |
| 241 | gaacaaactg aggggctgcc cggctgcaga caggaggaga cagagaggat ctattttagg |
| 301 | gtggcaagtg cctacctacc ctaagcgagc aattccacgt tggggagaag ccagcagagg |
| 361 | ttgggaaagg gtgggagtcc aagggagccc ctgcgcaacc ccctcaggaa taaaactccc |
| 421 | cagccagggt gtcgcaaggg ctgccgttgt gatccgcagg gggtgaacgc aaccgcgacg |
| 481 | gctgatcgtc tgtggctggg ttggcgtttg gagcaagaga aggaggagca ggagaaggag |
| 541 | ggagctggag gctggaagcg tttgcaagcg gcggcggcag caacgtggag taaccaagcg |
| 601 | ggtcagcgcg cgcccgccag ggtgtaggcc acggcgcgca gctcccagag caggatccgc |
| 661 | gccgcctcag cagcctctgc ggcccctgcg gcacccgacc gagtaccgag cgccctgcga |
| 721 | agcgcaccct cctccccgcg gtgcgctggg ctcgccccca gcgcgcgcac acgcacacac |
| 781 | acacacacac acacacacgc acgcacacac gtgtgcgctt ctctgctccg gagctgctgc |
| 841 | tgctcctgct ctcagcgccg cagtggaagg caggaccgaa ccgctccttc tttaaatata |
| 901 | taaatttcag cccaggtcag cctcggcggc ccccctcacc gcgctcccgg cgcccctccc |
| 961 | gtcagttcgc cagctgccag ccccgggacc ttttcatctc ttcccttttg gccggaggag |
| 1021 | ccgagttcag atccgccact ccgcacccga gactgacaca ctgaactcca cttcctcctc |
| 1081 | ttaaatttat ttctacttaa tagccactcg tctctttttt tccccatctc attgctccaa |
| 1141 | gaattttttt cttcttactc gccaaagtca gggttccctc tgcccgtccc gtattaatat |
| 1201 | ttccactttt ggaactactg gccttttctt tttaaaggaa ttcaagcagg atacgttttt |
| 1261 | ctgttgggca ttgactagat tgtttgcaaa agtttcgcat caaaaacaac aacaacaaaa |
| 1321 | aaccaaacaa ctctccttga tctatacttt gagaattgtt gatttctttt ttttattctg |
| 1381 | acttttaaaa acaacttttt tttccacttt tttaaaaaat gcactactgt gtgctgagcg |
| 1441 | cttttctgat cctgcatctg gtcacggtcg cgctcagcct gtctacctgc agcacactcg |
| 1501 | atatggacca gttcatgcgc aagaggatcg aggcgatccg cgggcagatc ctgagcaagc |
| 1561 | tgaagctcac cagtccccca gaagactatc ctgagcccga ggaagtcccc ccggaggtga |
| 1621 | tttccatcta caacagcacc agggacttgc tccaggagaa ggcgagccgg agggcggccg |
| 1681 | cctgcgagcg cgagaggagc gacgaagagt actacgccaa ggaggtttac aaaatagaca |
| 1741 | tgccgccctt cttcccctcc gaaactgtct gcccagttgt tacaacaccc tctggctcag |
| 1801 | tgggcagctt gtgctccaga cagtcccagg tgctctgtgg gtaccttgat gccatcccgc |
| 1861 | ccactttcta cagaccctac ttcagaattg ttcgatttga cgtctcagca atggagaaga |
| 1921 | atgcttccaa tttggtgaaa gcagagttca gagtctttcg tttgcagaac ccaaaagcca |
| 1981 | gagtgcctga acaacggatt gagctatatc agattctcaa gtccaaagat ttaacatctc |
| 2041 | caacccagcg ctacatcgac agcaaagttg tgaaaacaag agcagaaggc gaatggctct |
| 2101 | ccttcgatgt aactgatgct gttcatgaat ggcttcacca taaagacagg aacctgggat |
| 2161 | ttaaaataag cttacactgt ccctgctgca cttttgtacc atctaataat tacatcatcc |
| 2221 | caaataaaag tgaagaacta gaagcaagat ttgcaggtat tgatggcacc tccacatata |
| 2281 | ccagtggtga tcagaaaact ataaagtcca ctaggaaaaa aaacagtggg aagaccccac |
| 2341 | atctcctgct aatgttattg ccctcctaca gacttgagtc acaacagacc aaccggcgga |
| 2401 | agaagcgtgc tttggatgcg gcctattgct ttagaaatgt gcaggataat tgctgcctac |
| 2461 | gtccacttta cattgatttc aagagggatc tagggtggaa atggatacac gaacccaaag |
| 2521 | ggtacaatgc caacttctgt gctggagcat gcccgtattt atggagttca gacactcagc |
| 2581 | acagcagggt cctgagctta tataatacca taaatccaga agcatctgct tctccttgct |
| 2641 | gcgtgtccca agatttagaa cctctaacca ttctctacta cattggcaaa acacccaaga |
| 2701 | ttgaacagct ttctaatatg attgtaaagt cttgcaaatg cagctaaaat tcttggaaaa |
| 2761 | gtggcaagac caaaatgaca atgatgatga taatgatgat gacgacgaca acgatgatgc |
| 2821 | ttgtaacaag aaaacataag agagccttgg ttcatcagtg ttaaaaaatt tttgaaaagg |
| 2881 | cggtactagt tcagacactt tggaagtttg tgttctgttt gttaaaactg gcatctgaca |
| 2941 | caaaaaaagt tgaaggcctt attctacatt tcacctactt tgtaagtgag agagacaaga |
| 3001 | agcaaatttt ttttaaagaa aaaaataaac actggaagaa tttattagtg ttaattatgt |
| 3061 | gaacaacgac aacaacaaca acaacaacaa acaggaaaat cccattaagt ggagttgctg |
| 3121 | tacgtaccgt tcctatcccg cgcctcactt gatttttctg tattgctatg caataggcac |
| 3181 | ccttcccatt cttactctta gagttaacag tgagttattt attgtgtgtt actatataat |
| 3241 | gaacgtttca ttgcccttgg aaaataaaac aggtgtataa agtggagacc aaatactttg |
| 3301 | ccagaaactc atggatggct taaggaactt gaactcaaac gagccagaaa aaaagaggtc |
| 3361 | atattaatgg gatgaaaacc caagtgagtt attatatgac cgagaaagtc tgcattaaga |
| 3421 | taaagaccct gaaaacacat gttatgtatc agctgcctaa ggaagcttct tgtaaggtcc |
| 3481 | aaaaactaaa aagactgtta ataaaagaaa ctttcagtca gaataagtct gtaagttttt |
| 3541 | ttttttcttt ttaattgtaa atggttcttt gtcagtttag taaaccagtg aaatgttgaa |
| 3601 | atgttttgac atgtactggt caaacttcag accttaaaat attgctgtat agctatgcta |
| 3661 | taggtttttt cctttgtttt ggtatatgta accataccta tattattaaa atagatggat |
| 3721 | atagaagcca gcataattga aaacacatct gcagatctct tttgcaaact attaaatcaa |
| 3781 | aacattaact actttatgtg taatgtgtaa atttttacca tattttttat attctgtaat |
| 3841 | aatgtcaact atgatttaga ttgacttaaa tttgggctct ttttaatgat cactcacaaa |
| 3901 | tgtatgtttc ttttagctgg ccagtacttt tgagtaaagc ccctatagtt tgacttgcac |
| 3961 | tacaaatgca tttttttttt aataacattt gccctacttg tgctttgtgt ttctttcatt |
| 4021 | attatgacat aagctacctg ggtccacttg tcttttcttt tttttgtttc acagaaaaga |
| 4081 | tgggttcgag ttcagtggtc ttcatcttcc aagcatcatt actaaccaag tcagacgtta |
| 4141 | acaaattttt atgttaggaa aaggaggaat gttatagata catagaaaat tgaagtaaaa |
| 4201 | tgttttcatt ttagcaagga tttagggttc taactaaaac tcagaatctt tattgagtta |
| 4261 | agaaaagttt ctctaccttg gtttaatcaa tatttttgta aaatcctatt gttattacaa |
| 4321 | agaggacact tcataggaaa catctttttc tttagtcagg tttttaatat tcagggggaa |
| 4381 | attgaaagat atatatttta gtcgattttt caaaagggga aaaaagtcca ggtcagcata |
| 4441 | agtcattttg tgtatttcac tgaagttata aggtttttat aaatgttctt tgaaggggaa |
| 4501 | aaggcacaag ccaatttttc ctatgatcaa aaaattcttt ctttcctctg agtgagagtt |
| 4561 | atctatatct gaggctaaag tttaccttgc tttaataaat aatttgccac atcattgcag |
| 4621 | aagaggtatc ctcatgctgg ggttaataga atatgtcagt ttatcacttg tcgcttattt |
| 4681 | agctttaaaa taaaaattaa taggcaaagc aatggaatat ttgcagtttc acctaaagag |
| 4741 | cagcataagg aggcgggaat ccaaagtgaa gttgtttgat atggtctact tcttttttgg |
| 4801 | aatttcctga ccattaatta aagaattgga tttgcaagtt tgaaaactgg aaaagcaaga |
| 4861 | gatgggatgc cataatagta aacagccctt gtgttggatg taacccaatc ccagatttga |
| 4921 | gtgtgtgttg attatttttt tgtcttccac ttttctatta tgtgtaaatc acttttattt |
| 4981 | ctgcagacat tttcctctca gataggatga cattttgttt tgtattattt tgtctttcct |
| 5041 | catgaatgca ctgataatat tttaaatgct ctattttaag atctcttgaa tctgtttttt |
| 5101 | ttttttttaa tttgggggtt ctgtaaggtc tttatttccc ataagtaaat attgccatgg |
| 5161 | gaggggggtg gaggtggcaa ggaaggggtg aagtgctagt atgcaagtgg gcagcaatta |
| 5221 | tttttgtgtt aatcagcagt acaatttgat cgttggcatg gttaaaaaat ggaatataag |
| 5281 | attagctgtt ttgtattttg atgaccaatt acgctgtatt ttaacacgat gtatgtctgt |
| 5341 | ttttgtggtg ctctagtggt aaataaatta tttcgatgat atgtggatgt ctttttccta |
| 5401 | tcagtaccat catcgagtct agaaaacacc tgtgatgcaa taagactatc tcaagctgga |
| 5461 | aaagtcatac cacctttccg attgccctct gtgctttctc ccttaaggac agtcacttca |
| 5521 | gaagtcatgc tttaaagcac aagagtcagg ccatatccat caaggataga agaaatccct |
| 5581 | gtgccgtctt tttattccct tatttattgc tatttggtaa ttgtttgaga tttagtttcc |
| 5641 | atccagcttg actgccgacc agaaaaaatg cagagagatg tttgcaccat gctttggctt |
| 5701 | tctggttcta tgttctgcca acgccagggc caaaagaact ggtctagaca gtatcccctg |
| 5761 | tagccccata acttggatag ttgctgagcc agccagatat aacaagagcc acgtgctttc |
| 5821 | tggggttggt tgtttgggat cagctacttg cctgtcagtt tcactggtac cactgcacca |
| 5881 | caaacaaaaa aacccaccct atttcctcca atttttttgg ctgctaccta caagaccaga |
| 5941 | ctcctcaaac gagttgccaa tctcttaata aataggatta ataaaaaaag taattgtgac |
| 6001 | tcaaaaaaaa aaaaaa |

An exemplary human WNT3 amino acid sequence is set forth below (SEQ ID NO: 47; GenBank Accession No: BAB70502.1, Version 1, incorporated herein by reference):

| | |
|---|---|
| 1 | mephllglll glllggtrvl agypiwwsla lgqqytslgs qpllcgsipg lvpkqlrfcr |
| 61 | nyieimpsva egvklgiqec qhqfrgrrwn cttiddslai fgpvldkatr esafvhaias |
| 121 | agvafavtrs caegtsticg cdshhkgppg egwkwggcse dadfgvlvsr efadarenrp |
| 181 | darsamnkhn neagrttild hmhlkckchg lsgscevktc wwaqpdfrai gdflkdkyds |
| 241 | asemvvekhr esrgwvetlr akyslfkppt erdlvyyens pnfcepnpet gsfgtrdrtc |
| 301 | nvtshgidgc dllccgrghn trtekrkekc hcifhwccyv scqeciriyd vhtck |

An exemplary human WNT3 nucleic acid sequence is set forth below (SEQ ID NO: 48; GenBank Accession No: NM_030753.4, Version 4, incorporated herein by reference):

| | |
|---|---|
| 1 | tgctccgcgc tgggctcggg aggggggcgg ctgcgggtgg aggtgcgctt ctgacaagcc |
| 61 | cgaaagtcat ttccaatctc aagtggactt tgttccaact attgggggcg tcgctccccc |
| 121 | tcttcatggt cgcgggcaaa cttcctcctc ggcgcctctt ctaatggagc cccacctgct |
| 181 | cgggctgctc ctcggcctcc tgctcggtgg caccagggtc ctcgctggct acccaatttg |
| 241 | gtggtccctg gccctgggcc agcagtacac atctctgggc tcacagcccc tgctctgcgg |
| 301 | ctccatccca ggcctggtcc ccaagcaact gcgcttctgc cgcaattaca tcgagatcat |
| 361 | gcccagcgtg gccgagggcg tgaagctggg catccaggag tgccagcacc agttccgggg |
| 421 | ccgccgctgg aactgcacca ccatagatga cagcctggcc atctttgggc ccgtcctcga |
| 481 | caaagccacc cgcgagtcgg ccttcgttca cgccatcgcc tcggccggcg tggccttcgc |
| 541 | cgtcacccgc tcctgcgccg agggcacctc caccatttgc ggctgtgact cgcatcataa |
| 601 | ggggccgcct ggcgaaggct ggaagtgggg cggctgcagc gaggacgctg acttcggcgt |
| 661 | gttagtgtcc agggagttcg cggatgcgcg cgagaacagg ccggacgcgc gctcggccat |
| 721 | gaacaagcac aacaacgagg cgggccgcac gactatcctg gaccacatgc acctcaaatg |
| 781 | caagtgccac gggctgtcgg gcagctgtga ggtgaagacc tgctggtggg cgcagcctga |
| 841 | cttccgtgcc atcggtgact tcctcaagga caagtatgac agcgcctcgg agatggtagt |
| 901 | agagaagcac cgtgagtccc gaggctgggt ggagaccctc cgggccaagt actcgctctt |
| 961 | caagccaccc acggagaggg acctggtcta ctacgagaac tcccccaact tttgtgagcc |
| 1021 | caacccagag acgggttcct ttggcacaag ggaccggact tgcaatgtca cctcccacgg |
| 1081 | catcgatggc tgcgatctgc tctgctgtgg ccggggccac aacacgagga cggagaagcg |
| 1141 | gaaggaaaaa tgccactgca tcttccactg gtgctgctac gtcagctgcc aggagtgtat |
| 1201 | tcgcatctac gacgtgcaca cctgcaagta gggcaccagg gcgctgggaa ggggtgaagt |
| 1261 | gtgtggctgg gcggattcag cgaagtctca tgggaagcag gacctagagc cgggcacagc |
| 1321 | cctcagcgtc agacagcaag gaactgtcac cagccgcacg cgtggtaaat gacccagacc |
| 1381 | caactcgcct gtggacgggg aggctctccc tctctctcat cttacatttc tcaccctact |
| 1441 | ctggatggtg tgtggttttt aaagaagggg gctttctttt tagttctcta gggtctgata |
| 1501 | ggaacagacc tgaggcttat ctttgcacat gttaaagaaa ataaaaatga aaaaaaattt |
| 1561 | gactccaaca gaacaggctg ggctaatgtg agctctcagc ctggcagtca agacatcagc |
| 1621 | atgggcaagg ttctgtttcc aaactgctgc ttctggtgac attccaagac gcctggaggg |
| 1681 | tgggagtcag gaagtaggac acacccctgc agtctccttt tcttggtcca ctcccattca |
| 1741 | aatttgagct aatttctcat tctgataaaa gccataggtt tagctaggat gaagtggtag |
| 1801 | gaaggtccgt ggcagttgtt agagtaggat ttggagtttg gaagaactgg cagctcaggg |
| 1861 | tggcctggtc agccgtttga agagcagcca tgtgttcttc tcagtctcat tttctctata |
| 1921 | accctgttct gcacgagggg cagtcagatc tcaaaatctt tttctaccat tctgcagttt |
| 1981 | ccaccgtcaa tgcagttttt tttttgtttt tttgtttttt tttttttttg gtggtagtgg |
| 2041 | accttgtaaa taggctatgt aagggggcaa gtcttctcta gctcaaatgg cttcctaaat |
| 2101 | aaataagcgg tatcttcaga aggggccatt cagtccttcc cagccctgct cacctgcaga |
| 2161 | ttctctgtac aaataactcc aggtagagca gttggactcc aggtcacctt agtataagtt |
| 2221 | agacaaaggg tccgtgaggg agtagccatc aattcctgaa attccaactt tgtgactagc |
| 2281 | agatggggag gatgaaaacc atccctttgc ttcctctcca atacggaccc atcttactgt |
| 2341 | gtcctttcct ctctggggcc aatgtgagta aacacagaca cagagttctt tcccccagct |
| 2401 | cttcctccct cacctgcatg ctgagatagc ttccatccat gcagttccca aggatctgga |
| 2461 | ttagaagttc aaaggggaac cagcagtcac ctactccctt aggtgaagca tctcacggct |
| 2521 | gagttctccc tgaggcatac tggtccagct gagcgtccta gagaaagcta gcaaaaggga |
| 2581 | ggcacatgga tttcacagta tgaattggtt caacaactgt cttagggaga atcagaaaga |
| 2641 | agagatgcag caggggaatg agcagaacaa agatttttct ttctccccct tctctctggg |
| 2701 | gtctacctaa ccctgaccta aaataccagg gcagcgatct cccagctggt gcaggtgggc |
| 2761 | ttgccaagat ggtcgtccag gagcccgcct tcacttctaa atctgctggc cacaagccct |
| 2821 | gctaaagata cacatctcac cccctccgcc aagtctgaaa tgcccctccc catctcacct |
| 2881 | tagactgaaa agttttaaat catgtcaact ggataatact tgctttatgt gagaatactt |
| 2941 | cagcagaatg gatacgaatt ttcaaaacaa tcttttcata tctatgtatt ctatattaaa |
| 3001 | agtgataaag tcatgtttct ggggcgtatt caagtagctg acaagtaatt atttaataat |
| 3061 | agtacatgag tgcattgtaa tgattctcgc cgtagtcagg taatagtatc caaccgaaat |
| 3121 | ttcctaccaa cctgctgtat ccaaagtttt gtaaaaagtt gtagaagttg ttgatctttt |
| 3181 | tgattttata ttcaaaaagt ctctttttat aaatattatt tattatacaa tgtatatacc |
| 3241 | tttgagttaa ctaagattat atattatata aatatatata tatttggaga aaatatattt |
| 3301 | catcatgcag tttttttctg ttaagtcatt aaagagaagg taaacaaacc taaaaaaaaa |
| 3361 | aaaaaaaaaa aaaaaaaaaa aaaaa |

An exemplary human HOXD13 amino acid sequence is set forth below (SEQ ID NO: 49; GenBank Accession No: AAC51635.1, Version 1, incorporated herein by reference):

| | |
|---|---|
| 1 | mdglradggg aggapassss ssvaaaaasg qcrgflsapv fagthsgraa aaaaaaaaaa |
| 61 | aaasgfaypg tsertgssss ssssavvaar peappakecp aptpaaaaaa ppsapalgyg |
| 121 | yhfgngyysc rmshgvglqq nalkssphas lggfpvekym dvsglasssv panevparak |
| 181 | evsfyqgyts pyqhvpgyid mvstfgsgep rheayismeg yqswtlangw nsqvyctkdq |
| 241 | pqgshfwkss fpgdvalnqp dmcvyrrgrk krvpytklql keleneyain kfinkdkrrr |
| 301 | isaatnlser qvtiwfqnrr vkdkkivskl kdtvs |

An exemplary human HOXD13 nucleic acid sequence is set forth below (SEQ ID NO: 50; GenBank Accession No: NM_000523.3, Version 3, incorporated herein by reference):

| | |
|---|---|
| 1 | gagaaaggag aggagggagg aggcgcgccg cgccatggtg tcctgcgcgg ggccagggcc |
| 61 | agggccgggg ccgggccagg ccgggccatg agccgcgccg ggagctggga catggacggg |
| 121 | ctgcgggcag acggcggggg cgccggtggc gccccggcct cttcctcctc ctcatcggtg |
| 181 | gcggcggcgg cggcgtcagg ccagtgccgc ggctttctct ccgcgcctgt gttcgccggg |
| 241 | acgcattcgg ggcgggcggc ggcggcggca gcggcggctg cggcggcggc ggcggcagcc |
| 301 | tccggctttg cgtaccccgg gacctctgag cgcacgggct cttcctcgtc gtcgtcctct |
| 361 | tctgccgttg tagcggcgcg cccggaggct cccccagcca aagagtgccc agcacccacg |
| 421 | cctgcagcgg ccgctgcagc gcccccgagc gctccagcgc tgggctacgg ctaccacttc |
| 481 | ggcaacggct actacagctg ccgtatgtcg cacggcgtgg gcttacagca gaatgcgctc |
| 541 | aagtcatcgc cgcacgcctc gctgggaggc tttcccgtgg agaagtacat ggacgtgtca |
| 601 | ggcctggcga gcagcagcgt accggccaac gaggtgccag cgcgagccaa ggaggtatcc |
| 661 | ttctaccagg gctatacgag cccttaccag cacgtgcccg gctatatcga catggtgtcc |
| 721 | actttcggct ccggggagcc tcggcacgag gcctacatct ccatggaggg gtaccagtcc |
| 781 | tggacgctgg ctaacgggtg gaacagccag gtgtactgca ccaaggacca gccacagggg |
| 841 | tcccactttt ggaaatcttc ctttccaggg gatgtggctc taaatcagcc ggacatgtgc |
| 901 | gtctaccgaa gagggaggaa gaagagagtg ccttacacca aactgcagct taaagaactg |
| 961 | gagaacgagt atgccattaa caaattcatt aacaaggaca agcggcggcg tatctcggct |
| 1021 | gctacgaacc tatctgagag acaagtgacc atttggtttc agaaccgaag agtgaaggac |
| 1081 | aagaaaattg tctccaagct caaagatact gtctcctgat gtggtccagg ttggccacag |
| 1141 | acagcttaga agccattcgg ttgtctccaa aaggcctttg gaaagacttg aatatgtatt |
| 1201 | taattccccc caccccctgc caatggtggc aaattttgtg aattgttttt ctctcttccc |
| 1261 | cttatctggc tctaaaacct tctgctgccc aacctgactt tgtagttctg atttttactt |
| 1321 | gtttattatt ggttttgttc ttgcctaggg tttttaaaat atctgttttt aatgttttgt |
| 1381 | ttctccctcc aggccagtat aaagggactt gaagtatttt ttaataatcc gccccccaat |
| 1441 | gaacttcaga agtgccattc tgatttaagg gtttttttaa aaaattactt tatttgttca |
| 1501 | ttcccagcac tgattatctt cataatccat taggacagaa tggttttcag tcgttcatat |
| 1561 | cctgtaatta ggtaattgaa tcattagctc tcagcagttg ccctgaggca agtggaaagg |
| 1621 | caggcagtgc tctggggtca ccgagaaagt ctaaaaacag gaggctgaag gtactgtgat |
| 1681 | ggctttaaaa atggccacct tattaaatag ggattgtatc aatattgaaa tgaagacaat |
| 1741 | ctttccaact ttgggtgttt cacttgctgt tttaattgtt tgtttttaac actttgtagg |
| 1801 | tttgtgtttt cataatcttt aatttgaaac tcatgtgtcc tcatggatcg tggatgcctt |
| 1861 | catttcttga gctctcaatg cagacattta aatggctgca atcagtagag tgacccgcgg |
| 1921 | atggcataaa tgcacctcct tttcttggcc ttggatctat gggtctggga ttgtggtcat |
| 1981 | ctcctcaatc ctcaaaaaga ggctgaatca atgtggccgt gggtgggaac ttacatacag |
| 2041 | aacccaatga agaacttgac tgtctaaaca agggggcctc gcatggagct gtaaagcatc |
| 2101 | taacaaatat gaaaaatgtg aagttccaag gtccaagaag aaaaataatg atgtttctga |
| 2161 | aagtgatgat aaataattac ttttaaagtg ctgcatattt atacaattga gagattattt |
| 2221 | ttgtaaatgc aatgtctgtg agctgggata catgggcagt gcttcagaca tttaaaaatc |
| 2281 | actttttact cctagggaga tgccaataaa cagaactctt ttgtttcaaa aaaaaaaaaa |
| 2341 | a |

An exemplary human HOXD11 amino acid sequence is set forth below (SEQ ID NO: 51; GenBank Accession No: AAI09395.1, Version 1, incorporated herein by reference):

| | |
|---|---|
| 1 | mylpgcayyv apsdfaskps flsqpsscqm tfpyssnler gggspctkat pgsepkgaae |
| 61 | gsggdgegpp geagaeksss avapqrsrkk rcpytkyqir elerefffnv yinkekrlql |
| 121 | srmlnltdrq vkiwfqnrrm kekklnrdrl qyftgnplf |

An exemplary human HOXD11 nucleic acid sequence is set forth below (SEQ ID NO: 52; GenBank Accession No: NM_021192.2, Version 2, incorporated herein by reference):

| | |
|---|---|
| 1 | atgaacgact ttgacgagtg cggccagagc gcagccagca tgtacctgcc gggctgcgcc |
| 61 | tactatgtgg ccccgtctga cttcgctagc aagccttcgt tcctttccca accgtcgtcc |
| 121 | tgccagatga ctttccccta ctcttccaac ctggctccgc acgtccagcc cgtgcgcgaa |
| 181 | gtggccttcc gcgactacgg cctggagcgc gccaagtggc cgtaccgcgg cggcggcggc |
| 241 | ggcggcagcg cggggggcgg cagcagcggg ggcggccccg gcgggggcgg cggcggcgcg |
| 301 | gggggctacg ctccctacta cgcggcggcg gcggcggcgg ctgcggcggc cgcggcggcc |
| 361 | gaggaggcgg ccatgcaacg cgagcttctc ccgcccgcgg gccgccggcc ggacgtgctc |
| 421 | ttcaaggcgc ctgagccggt gtgcgctgcg ccggggccgc cgcacggccc cgcgggcgcc |
| 481 | gcctccaact tctacagcgc ggtgggccgc aatggcatct tgccacaggg cttcgaccag |
| 541 | ttctacgagg cagcgcccgg gcccccgttc gccgggccgc agcccccgcc gccacccgcg |
| 601 | ccgccacagc ccgagggcgc agccgacaag ggcgacccca ggaccggggc tggtggcggc |
| 661 | gggggcagtc cctgcaccaa ggcgacccct ggctcggagc ccaagggggc agcagaaggc |
| 721 | agcggtggcg acggcgaggg ccccccggga gaggcggggg ccgagaagag cagcagcgca |
| 781 | gttgcccccc agcggtcccg gaaaaagcgc tgtccctata ccaagtacca gatccgcgaa |
| 841 | ctggaacgcg agtttttctt taacgtgtac ataaacaaag agaaaagact tcaactctct |
| 901 | cggatgctca acctcactga ccggcaagtc aaaatctggt tccagaatcg caggatgaaa |
| 961 | gaaaagaaac tgaacagaga ccgtctgcag tatttcactg gaaacccctt attttgagag |
| 1021 | ctccaggaag cgccctcacc ccagccccac tcacccaccc tccttcccac cagcctgctc |
| 1081 | tccgcaggcc cactgtcctt gggtttaatg acgtctcttc tctgtggaac ttcacgattc |
| 1141 | cttcccacgg tcaactcggg acctcccagc gaccactgca gcctgcggac gaggccggga |
| 1201 | cttggccgag cggatcctaa taaggggaaa atggtaaatg caaacgtccc gttacaattt |
| 1261 | taccgccagt gtgctgtcgt tccccctccc cctctccgag tcctcgtggg gacacggcgg |
| 1321 | ggtctgtagg aagttgggcc gggttggggg ttgctagaag gcgctggtgt tttgctctga |
| 1381 | gttttaagag atcccttcct tcctcttcgg tgaatgcagg ttatttaaac tttgggaaat |
| 1441 | gtacttttag tctgtcatat caa |

An exemplary human HOXA2 nucleic acid sequence is set forth below (SEQ ID NO: 53; GenBank Accession No: NM_006726.1, Version 1, incorporated herein by reference):

| | |
|---|---|
| 1 | mnyefereig finsqpslae cltsfppvad tfqsssikts tlshstlipp pfeqtipsln |
| 61 | pgshprhgag grpkpspags rgspvpagal qppeypwmke kkaakktall paaaaaataa |
| 121 | atgpaclshk esleiadgsg ggsrrlrtay tntqllelek efhfnkylcr prrveiaall |
| 181 | dlterqvkvw fqnrrmkhkr qtqckenqns egkcksleds ekveedeeek tlfeqalsvs |
| 241 | galleregyt fqgnalsqqq apnghngdsq sfpvspltsn eknlkhfqhq sptvpnclst |
| 301 | mgqncgagln ndspealevp slqdfsvfst dsclqlsdav spslpgslds pvdisadsld |
| 361 | fftdtlttid lqhlny |

An exemplary human HOXA2 nucleic acid sequence is set forth below (SEQ ID NO: 54; GenBank Accession No: NM_006735.3, Version 3, incorporated herein by reference):

| | |
|---|---|
| 1 | tcttttgatt aaagcccaaa ttgtcattgg gcagaagcaa tcatgtgaca gccaattcgg |
| 61 | tccaatttca accttgtctc catgaattca atagtttaat agtagcgcgg tccccatacg |
| 121 | gctgtaatca gtgaattaga aaaaaaacac cctagcagcg atattctatg atagattttt |
| 181 | tttcctctgc gctcgccttt ttcctaggcc ttgccccccc aaagcccctc caaaagaggg |
| 241 | aactttttct ctgagggggc tccaaggaga aggccatgaa ttacgaattt gagcgagaga |
| 301 | ttggttttat caatagccag ccgtcgctcg ctgagtgcct gacatctttt ccccctgtcg |
| 361 | ctgatacatt tcaaagttca tcaatcaaga cctcgacgct ttcacactcg acactgattc |
| 421 | ctcctccttt tgagcagacc attcccagcc tgaaccccgg cagtcaccct cgccacggcg |
| 481 | ctggcggccg ccccaagccg agccccgcgg gcagccgcgg cagcccggtg cccgccggcg |
| 541 | ccctgcagcc gcccgagtac ccctggatga aggagaagaa ggcggccaag aaaaccgcac |
| 601 | ttctgccggc cgccgccgcc gccgccaccg ccgcagccac cggccctgct tgcctcagcc |
| 661 | acaaagaatc cctggaaatc gccgatggca gcggcggggg atcgcggcgc ctgagaactg |
| 721 | cttacaccaa cacacagctt ctagagctgg aaaaagaatt tcatttcaac aagtaccttt |
| 781 | gcagaccccg aagggtggag attgcagcgc tgctggattt gactgagaga caagtgaaag |
| 841 | tgtggtttca gaaccggagg atgaagcaca agaggcagac ccagtgcaag gaaaaccaaa |
| 901 | acagcgaagg gaaatgtaaa agccttgagg actccgagaa agtagaggag gacgaggaag |
| 961 | agaagacgct ctttgagcaa gcccttagcg tctctggggc ccttctggag agggaaggct |
| 1021 | acacttttca gcaaaatgcc ctctctcagc agcaggctcc caatggacac aatggcgact |
| 1081 | cccaaagttt cccagtctcg cctttaacca gcaatgagaa aaatctgaaa cattttcagc |
| 1141 | accagtcacc cactgttccc aactgcttgt caacaatggg ccagaactgt ggagctggcc |
| 1201 | taaacaatga cagtcctgag gcccttgagg tcccctcttt gcaggacttt agcgttttct |
| 1261 | ccacagattc ctgcctgcag ctttcagatg cagtttcacc cagtttgcca ggttccctcg |
| 1321 | acagtcccgt agatatttca gctgacagct tagacttttt tacagacaca ctcaccacaa |
| 1381 | tcgacttgca gcatctgaat tactaaaaac attaaagcaa aacaaagcat caccaaacaa |
| 1441 | aaactccttt gaccaggtgg ttttgccttc ttttatttgg gagtttattt tttattttct |
| 1501 | tcttgaccta ccccttccct cctttaagtg ttgaggattt tctgtttagt gattccctga |
| 1561 | cccagtttca aacagagcca tcttttacag attattttgg agttttagtt gttttaaacc |
| 1621 | taactcaaca accctttatg tgattcctga gagcagtatg aggcctgcaa gaaagtgatc |
| 1681 | atataattgt atcttcactt tctttttatt tttgtattac attgggatgc attgtcatgc |
| 1741 | atattttttg tagaataaat tctcctttgc tataagtaaa aaaaaaaaaa a |

An exemplary human HOXA5 amino acid sequence is set forth below (SEQ ID NO: 55; GenBank Accession No: P20719.2, Version 2, incorporated herein by reference):

| | |
|---|---|
| 1 | mssyfvnsfc grypngpdyq lhnygdhssv seqfrdsasm hsgrygygyn gmdlsvgrsg |
| 61 | sghfgsgera rsyaasasaa paeprysqpa tsthspqpdp lpcsavapsp gsdshhggkn |
| 121 | slsnssgasa dagsthissr egvgtasgae edapasseqa saqsepspap paqpqiypwm |
| 181 | rklhishdni ggpegkrart aytryqtlel ekefhfnryl trrrrieiah alclserqik |
| 241 | iwfqnrrmkw kkdnklksms maaaggafrp |

An exemplary human HOXA5 nucleic acid sequence is set forth below (SEQ ID NO: 56; GenBank Accession No: NM_019102.3, Version 3, incorporated herein by reference):

| | |
|---|---|
| 1 | gggtgctata gacgcacaaa cgaccgcgag ccacaaatca agcacacata tcaaaaaaca |
| 61 | aatgagctct tattttgtaa actcattttg cggtcgctat ccaaatggcc cggactacca |
| 121 | gttgcataat tatggagatc atagttccgt gagcgagcaa ttcagggact cggcgagcat |
| 181 | gcactccggc aggtacggct acggctacaa tggcatggat ctcagcgtcg gccgctcggg |
| 241 | ctccggccac tttggctccg gagagcgcgc ccgcagctac gctgccagcg ccagcgcggc |
| 301 | gcccgccgag cccaggtaca gccagccggc cacgtccacg cactctcctc agcccgatcc |
| 361 | gctgccctgc tccgccgtgg ccccctcgcc cggcagcgac agccaccacg gcgggaaaaa |
| 421 | ctccctaagc aactccagcg gcgcctcggc cgacgccggc agcacccaca tcagcagcag |
| 481 | agagggggtt ggcacggcgt ccggagccga ggaggacgcc cctgccagca gcgagcaggc |
| 541 | gagtgcgcag agcgagccga gcccggcgcc gcccgcccaa ccccagatct acccctggat |
| 601 | gcgcaagctg cacataagtc atgacaacat aggcggcccg gaaggcaaaa gggcccggac |
| 661 | ggcctacacg cgctaccaga ccctggagct ggagaaggag ttccacttca accgttacct |
| 721 | gacccgcaga aggaggattg aaatagcaca tgctctttgc ctctccgaga gacaaattaa |
| 781 | aatctggttc caaaaccgga gaatgaagtg gaaaaaagat aataagctga aaagcatgag |
| 841 | catggccgcg gcaggagggg ccttccgtcc ctgagtatct gagcgtttaa agtactgagc |
| 901 | agtattagcg gatcccgcgt agtgtcagta ctaaggtgac tttctgaaac tcccttgtgt |
| 961 | tccttctgtg aagaagccct gttctcgttg ccctaattca tcttttaatc atgagcctgt |
| 1021 | ttattgccat tatagcgcct gtataagtag atctgctttc tgttcatctc tttgtcctga |
| 1081 | atggctttgt cttgaaaaaa aatagatgtt ttaacttatt tatatgaagc aagctgtgtt |
| 1141 | acttgaagta actataacaa aaaaagaaaa gagaaaaaaa aacacacaaa aagtccccct |
| 1201 | tcaatctcgt ttagtgccaa tgttgtgtgt tgcactcaag ttgtttaact gtgcatgtgc |
| 1261 | gtggaagtgt tcctgtctca atagctccaa gctgttaaag atatttttat tcaaactacc |
| 1321 | tatattcctt gtgtaattaa tgctgttgta gaggtgactt gatgagacac aacttgttcg |
| 1381 | acgtgtagtg actagtgact ctgtgatgaa aactgtgact ccaagcggtg tgtccctgcg |
| 1441 | tgcctttata ggaccctttg cacgaactct ggaagtggct cttataagcg cagcttcagt |
| 1501 | gatgtatgtt tttgtgaaca aagttacaaa tattgtccaa gtctggctgt tttaagcaaa |
| 1561 | ctgtgatcag cttttttttt tttttttttt tttttgtatt tgtttttaag gaaaaaatac |
| 1621 | tgactggaac aaaaaataaa ctttctattg taagttc |

An exemplary human HOXD10 amino acid sequence is set forth below (SEQ ID NO: 57; GenBank Accession No: P28358.2, Version 2, incorporated herein by reference):

| | |
|---|---|
| 1 | msfpnsspaa ntflvdslis acrsdsfyss sasmymppps admgtygmqt cgllpslakr |
| 61 | evnhqnmgmn vhpyipqvds wtdpnrscri eqpvtqqvpt csfttnikee snccmysdkr |
| 121 | nklisaevps yqrlvpescp venpevpvpg yfrlsqtyat gktqeynnsp egsstvmlql |
| 181 | nprgaakpql saaqlqmekk mnepvsgqep tkvsqvespe akgglpeers claevsvssp |
| 241 | evqekeskee iksdtptsnw ltaksgrkkr cpytkhqtle lekeflfnmy ltrerrleis |
| 301 | ksvnltdrqv kiwfqnrrmk lkkmsrenri reltanltfs |

An exemplary human HOXD10 nucleic acid sequence is set forth below (SEQ ID NO: 58; GenBank Accession No: NM_002148.3, Version 3, incorporated herein by reference):

| | |
|---|---|
| 1 | cggggaatgt tttcctagag atgtcagcct acaaaggaca caatctctct tcttcaaatt |
| 61 | cttccccaaa atgtcctttc ccaacagctc tcctgctgct aatacttttt tagtagattc |
| 121 | cttgatcagt gcctgcagga gtgacagttt ttattccagc agcgccagca tgtacatgcc |
| 181 | accacctagc gcagacatgg ggacctatgg aatgcaaacc tgtggactgc tcccgtctct |
| 241 | ggccaaaaga gaagtgaacc accaaaatat gggtatgaat gtgcatcctt atatacctca |
| 301 | agtagacagt tggacagatc cgaacagatc ttgtcgaata gagcaacctg ttacacagca |
| 361 | agtccccact tgctccttca ccaccaacat taaggaagaa tccaattgct gcatgtattc |
| 421 | tgataagcgc aacaaactca tttcggccga ggtcccttcg taccagaggc tggtccctga |
| 481 | gtcttgtccc gttgagaacc ctgaggttcc cgtccctgga tattttagac tgagtcagac |
| 541 | ctacgccacc gggaaaaccc aagagtacaa taatagcccc gaaggcagct ccactgtcat |
| 601 | gctccagctc aaccctcgtg gcgcggccaa gccgcagctc tccgctgccc agctgcagat |
| 661 | ggaaaagaag atgaacgagc ccgtgagcgg ccaggagccc accaaagtct cccaggtgga |
| 721 | gagccccgag gccaaaggcg gccttcccga agagaggagc tgcctggctg aggtctccgt |
| 781 | gtccagtccc gaagtgcagg agaaggaaag caaagaggaa atcaagtctg atacaccaac |
| 841 | cagcaattgg ctcactgcaa agagtggcag aaagaagagg tgcccttaca ctaagcacca |
| 901 | aacgctggaa ttagaaaaag agttcttgtt caatatgtac ctcacccgcg agcgccgcct |
| 961 | agagatcagt aagagcgtta acctcaccga caggcaggtc aagatttggt ttcaaaaccg |
| 1021 | ccgaatgaaa ctcaagaaga tgagccgaga gaaccggatc cgagaactga ccgccaacct |
| 1081 | cacgttttct taggtctgag gccggtctga ggccggtcag aggccaggat tggagagggg |
| 1141 | gcaccgcgtt ccagggccca gtgctggagg actgggaaag cggaaacaaa accttcaccg |
| 1201 | ctctttgttt gttgttttgt tgtattttgt tttcctgcta gaatgtgact ttggggtcat |
| 1261 | tatgttcgtg ctgcaagtga tctgtaatcc ctatgagtat atatatatat atatatatat |
| 1321 | atatataaaa acttagcacg tgtaatttat tattttttca tcgtaatgca gggtaactat |
| 1381 | tattgcgcat tttcatttgg gtcttaactt attggaactg tagagcatcc atccatccat |
| 1441 | ccatccagca atgtgacttt ttcatgtctt tcctaacaca aaaggtctat gtgtgtggtt |
| 1501 | agtccatgaa ctcatggcat tttgaataca tccagtactt taaaaatgac atatatattt |
| 1561 | aaaaaaaaaa gattaagaaa acccacaagt tggagggagg gggacttaaa aagcacatta |
| 1621 | caatgtatct tttcacaaat gaatttagca gttgtccttg gtgagatggg atattggcga |
| 1681 | tttatgcctt gtagcctttc ccttgtggtg catctgtggt ttggtagaag tacaacagca |
| 1741 | acctgtcctt tctgtgcatg ttctggtcgc atgtataatg caataaactc tggaaatgag |
| 1801 | ttcaaaaaaa aaaa |

An exemplary human ANGPT1 amino acid sequence is set forth below (SEQ ID NO: 59; GenBank Accession No: AAI52420.1, Version 1, incorporated herein by reference):

| | |
|---|---|
| 1 | mtvflsfafl aailthigcs nqrrspensg rrynriqhgq caytfilpeh dgncresttd |
| 61 | qyntnalqrd aphvepdfss qklqhlehvm enytqwlqkl enyivenmks emaqiqqnav |
| 121 | qnhtatmlei gtsllsqtae qtrkltdvet qvlnqtsrle iqllenslst yklekqllqq |
| 181 | tneilkihek nsllehkile megkhkeeld tlkeekenlq glvtrqtyii qelekqlnra |
| 241 | ttnnsvlqkq qlelmdtvhn lvnlctkegv llkggkreee kpfrdcadvy qagfnksgiy |
| 301 | tiyinnmpep kkvfcnmdvn gggwtviqhr edgsldfqrg wkeykmgfgn psgeywlgne |
| 361 | fifaitsqrq ymlrielmdw egnraysqyd rfhignekqn yrlylkghtg tagkqsslil |
| 421 | hgadfstkda dndncmckca lmltggwwfd acgpsnlngm fytagqnhgk lngikwhyfk |
| 481 | gpsyslrstt mmirpldf |

An exemplary human ANGPT1 nucleic acid sequence is set forth below (SEQ ID NO: 60; GenBank Accession No: NM_001146.4, Version 4, incorporated herein by reference):

| | |
|---|---|
| 1 | gccctaagcc atcagcaatc cttagtatag gggcacactc atgcattcct gtcaagtcat |
| 61 | cttgtgaaag gctgcctgct tccagcttgg cttggatgtg caaccttaat aaaactcact |
| 121 | gaggtctggg agaaaatagc agatctgcag cagatagggt agaggaaagg gtctagaata |
| 181 | tgtacacgca gctgactcag gcaggctcca tgctgaacgg tcacacagag aggaaacaat |
| 241 | aaatctcagc tactatgcaa taaatatctc aagttttaac gaagaaaaac atcattgcag |
| 301 | tgaaataaaa aattttaaaa ttttagaaca aagctaacaa atggctagtt ttctatgatt |
| 361 | cttcttcaaa cgctttcttt gagggggaaa gagtcaaaca aacaagcagt tttacctgaa |
| 421 | ataaagaact agttttagag gtcagaagaa aggagcaagt tttgcgagag gcacggaagg |
| 481 | agtgtgctgg cagtacaatg acagttttcc tttcctttgc tttcctcgct gccattctga |
| 541 | ctcacatagg gtgcagcaat cagcgccgaa gtccagaaaa cagtgggaga agatataacc |
| 601 | ggattcaaca tgggcaatgt gcctacactt tcattcttcc agaacacgat ggcaactgtc |
| 661 | gtgagagtac gacagaccag tacaacacaa acgctctgca gagagatgct ccacacgtgg |
| 721 | aaccggattt ctcttcccag aaacttcaac atctggaaca tgtgatggaa aattatactc |
| 781 | agtggctgca aaaacttgag aattacattg tggaaaacat gaagtcggag atggcccaga |
| 841 | tacagcagaa tgcagttcag aaccacacgg ctaccatgct ggagatagga accagcctcc |
| 901 | tctctcagac tgcagagcag accagaaagc tgacagatgt tgagacccag gtactaaatc |
| 961 | aaacttctcg acttgagata cagctgctgg agaattcatt atccacctac aagctagaga |
| 1021 | agcaacttct tcaacagaca aatgaaatct tgaagatcca tgaaaaaaac agtttattag |
| 1081 | aacataaaat cttagaaatg gaaggaaaac acaaggaaga gttggacacc ttaaaggaag |
| 1141 | agaaagagaa ccttcaaggc ttggttactc gtcaaacata tataatccag gagctggaaa |
| 1201 | agcaattaaa cagagctacc accaacaaca gtgtccttca gaagcagcaa ctggagctga |
| 1261 | tggacacagt ccacaacctt gtcaatcttt gcactaaaga aggtgtttta ctaaagggag |
| 1321 | gaaaaagaga ggaagagaaa ccatttagag actgtgcaga tgtatatcaa gctggtttta |
| 1381 | ataaaagtgg aatctacact atttatatta ataatatgcc agaacccaaa aaggtgtttt |
| 1441 | gcaatatgga tgtcaatggg ggaggttgga ctgtaataca acatcgtgaa gatggaagtc |
| 1501 | tagatttcca aagaggctgg aaggaatata aaatgggttt tggaaatccc tccggtgaat |
| 1561 | attggctggg gaatgagttt atttttgcca ttaccagtca gaggcagtac atgctaagaa |
| 1621 | ttgagttaat ggactgggaa gggaaccgag cctattcaca gtatgacaga ttccacatag |
| 1681 | gaaatgaaaa gcaaaactat aggttgtatt taaaaggtca cactgggaca gcaggaaaac |
| 1741 | agagcagcct gatcttacac ggtgctgatt tcagcactaa agatgctgat aatgacaact |
| 1801 | gtatgtgcaa atgtgccctc atgttaacag gaggatggtg gtttgatgct tgtggcccct |
| 1861 | ccaatctaaa tggaatgttc tatactgcgg gacaaaacca tggaaaactg aatgggataa |
| 1921 | agtggcacta cttcaaaggg cccagttact ccttacgttc cacaactatg atgattcgac |
| 1981 | ctttagattt ttgaaagcgc aatgtcagaa gcgattatga aagcaacaaa gaaatccgga |
| 2041 | gaagctgcca ggtgagaaac tgtttgaaaa cttcagaagc aaacaatatt gtctcccttc |
| 2101 | cagcaataag tggtagttat gtgaagtcac caaggttctt gaccgtgaat ctggagccgt |
| 2161 | ttgagttcac aagagtctct acttggggtg acagtgctca cgtggctcga ctatagaaaa |
| 2221 | ctccactgac tgtcgggctt taaaaaggga agaaactgct gagcttgctg tgcttcaaac |
| 2281 | tactactgga ccttattttg gaactatggt agccagatga taaatatggt taatttcatg |
| 2341 | taaaacagaa aaaaagagtg aaaaagagaa tatacatgaa gaatagaaac aagcctgcca |
| 2401 | taatcctttg gaaaagatgt attataccag tgaaaaggtg ttatatctat gcaaacctac |
| 2461 | taacaaatta tactgttgca caattttgat aaaaatttag aacagcattg tcctctgagt |
| 2521 | tggttaaatg ttaatggatt tcagaagcct aattccagta tcatacttac tagttgattt |
| 2581 | ctgcttaccc atcttcaaat gaaaattcca tttttgtaag ccataatgaa ctgtagtaca |
| 2641 | tggacaataa gtgtgtggta gaaacaaact ccattactct gatttttgat acagttttca |
| 2701 | gaaaaagaaa tgaacataat caagtaagga tgtatgtggt gaaaacttac cacccccata |
| 2761 | ctatggtttt catttactct aaaaactgat tgaatgatat ataaatatat ttatagcctg |
| 2821 | agtaaagtta aaagaatgta aaatatatca tcaagttctt aaaataatat acatgcattt |
| 2881 | aatatttcct ttgatattat acaggaaagc aatattttgg agtatgttaa gttgaagtaa |
| 2941 | aagcaagtac tctggagcag ttcattttac agtatctact tgcatgtgta tacatacatg |
| 3001 | taacttcatt attttaaaaa tatttttaga actccaatac tcaccctgtt atgtcttgct |
| 3061 | aatttaaatt ttgctaatta actgaaacat gcttaccaga ttcacactgt tccagtgtct |
| 3121 | ataaaagaaa cactttgaag tctataaaaa ataaaataat tataaatatc attgtacata |
| 3181 | gcatgtttat atctgcaaaa aacctaatag ctaattaatc tggaatatgc aacattgtcc |
| 3241 | ttaattgatg caaataacac aaatgctcaa agaaatctac tatatccctt aatgaaatac |
| 3301 | atcattcttc atatatttct ccttcagtcc attcccttag gcaattttta atttttaaaa |
| 3361 | attattatca ggggagaaaa attggcaaaa ctattatatg taagggaaat atatacaaaa |
| 3421 | agaaaattaa tcatagtcac ctgactaaga aattctgact gctagttgcc ataaataact |
| 3481 | caatggaaat attcctatgg gataatgtat tttaagtgaa tttttggggt gcttgaagtt |
| 3541 | actgcattat tttatcaaga agtcttctct gcctgtaagt gtccaaggtt atgacagtaa |
| 3601 | acagttttta ttaaaacatg agtcactatg ggatgagaaa attgaaataa agctactggg |
| 3661 | cctcctctca taaaagagac agttgttggc aaggtagcaa taccagtttc aaacttggtg |
| 3721 | acttgatcca ctatgcctta atggtttcct ccatttgaga aaataaagct attcacattg |
| 3781 | ttaagaaaaa tactttttaa agtttaccat caagtctttt ttatatttat gtgtctgtat |
| 3841 | tctacccctt tttgccttac aagtgatatt tgcaggtatt ataccatttt tctattcttg |
| 3901 | gtggcttctt catagcaggt aagcctctcc ttctaaaaac ttctcaactg ttttcattta |
| 3961 | agggaaagaa aatgagtatt ttgtcctttt gtgttcctac agacactttc ttaaaccagt |
| 4021 | ttttggataa agaatactat ttccaaactc atattacaaa aacaaaataa aataataaaa |
| 4081 | aaagaaagca tgatatttac tgttttgttg tctgggtttg agaaatgaaa tattgtttcc |
| 4141 | aattatttat aataaatcag tataaaatgt tttatgattg ttatgtgtat tatgtaatac |
| 4201 | gtacatgttt atggcaattt aacatgtgta ttcttttaat tgtttcagaa taggataatt |
| 4261 | aggtattcga attttgtctt taaaattcat gtggtttcta tgcaaagttc ttcatatcat |
| 4321 | cacaacatta tttgatttaa ataaaattga aagtaatatt tgtgcaa |

An exemplary human ANG2 amino acid sequence is set forth below (SEQ ID NO: 61; GenBank Accession No: AAF21627.2, Version 2, incorporated herein by reference):

| | |
|---|---|
| 1 | maaaaaagps pgsgpgdspe gpegeaperr rkahgmlkly yglsegeaag rpagpdpldp |
| 61 | tdlngahfdp evyldklrre cplaqlmdse tdmvrqiral dsdmqtivye nynkfisatd |
| 121 | tirkmkndfr kmedemdrla tnmavitdfs arisatlqdr heritklagv hallrklqfl |
| 181 | felpsrltkc velgaygqav ryggragavl qqyqhlpsfr aiqddcqvit arlaqqlrqr |
| 241 | freggsgape qaecvellla lgepaeelce eflahargrl ekelrnleae lgpsppapdv |
| 301 | leftdhggsg fvgglcqvaa ayqelfaaqg pagaeklaaf arqlgsryfa lverrlageg |
| 361 | gggdnsllvr aldrfhrrlr apgallaaag ladaateive rvarerlghh lqglraaflg |
| 421 | cltdvrqala aprvagkegp glaellanva ssilshikas laavhlftak evsfsnkpyf |
| 481 | rgefcsqgvr eglivgfvhs mcqtaqsfcd spgekggatp palllllsrl cldyetatis |
| 541 | yiltltdeqf lvqdqfpvtp vstlcaeare tarrllthyv kvqglvisqm lrksvetrdw |
| 601 | lstleprnvr avmkrvvedt taidvqvgll yeegvrkaqs sdsskrtfsv ysssrqqgry |
| 661 | apsytpsapm dtnllsniqk lfseridvfs pvefnkvsvl tgiikislkt llecvrlrtf |
| 721 | grfglqqvqv dchflqlylw rfvadeelvh llldevvasa alrcpdpvpm epsvvevice |
| 781 | rg |

An exemplary human ANG2 nucleic acid sequence is set forth below (SEQ ID NO: 62; GenBank Accession No: AF024631.2, Version 2, incorporated herein by reference):

| | |
|---|---|
| 1 | ttcctttcca gcctcacgcc cgtgggctgc agttggaacg atggcggcgg cagctgccgc |
| 61 | cgggcctagc ccggggtctg gacctgggga ctccccagaa gggcccgagg gggaggctcc |
| 121 | ggagcgtcgg cggaaggcgc acgggatgct gaagctttac tacggcctct cggaagggga |
| 181 | ggcggcggga cgccccgcgg ggcccgaccc cctggacccg actgatctga acggggcgca |
| 241 | cttcgacccg gaagtttacc tagacaagct gcgtagagag tgccctctgg cccagttgat |
| 301 | ggacagtgag acggacatgg tgcggcagat ccgggctcta gacagcgaca tgcagaccct |
| 361 | ggtctatgag aactacaaca agttcatctc agccacagac accatccgga agatgaagaa |
| 421 | cgatttccgg aagatggagg atgagatgga ccggctggcc accaacatgg cagtgatcac |
| 481 | cgacttcagc gctcgcatca gcgccacgct gcaggaccgc cacgagcgca tcaccaagct |
| 541 | ggcaggggtc cacgcgctgc tgcggaagct gcagttcctc tttgagctgc cctcgcgcct |
| 601 | caccaagtgc gtggaactgg gcgcctatgg gcaggcggtg cgctaccagg gccgcgcgca |
| 661 | ggccgtgctg cagcagtacc aacacctgcc ctcgttccgc gccatccagg acgactgcca |
| 721 | ggtcatcacg gcccgcctgg cccagcagct gcggcagcgc tttagggagg gcggctcagg |
| 781 | cgccccggag caggcagagt gcgtggagct gctgctggcc ctgggcgagc ctgcggagga |
| 841 | gctgtgcgag gagttcctgg cgcacgcccg cggccggctg gagaaggagc tgagaaacct |
| 901 | ggaggccgag ctggggccct cacctccggc tcccgacgtg ttagagttca ccgaccatgg |
| 961 | aggcagtggc ttcgtgggcg gcctctgcca ggtggcggcg gcctaccagg agctgtttgc |
| 1021 | ggcccagggc ccagcaggtg ccgagaagct ggcggccttc gcccggcagc tgggcagccg |
| 1081 | ctattttgcg ctggtggagc ggcggctggc gcaggagcag ggtggtggtg acaactcact |
| 1141 | gctggtgcgg gcgctggacc gcttccaccg gcgcttgcgg gctcccgggg ccctgctggc |
| 1201 | cgctgccggg ctcgcagacg ctgccacgga gatcgtggaa cgagtggccc gcgagcgcct |
| 1261 | gggccaccac ctgcagggtc tccgggcggc cttcctgggc tgcctgacag acgtccgcca |
| 1321 | ggcgctggca gcacctcgcg tggctgggaa ggagggccct ggcctggccg agttgctggc |
| 1381 | caatgtggcc agctccatcc tgagccacat taaggcctct ctggcagcag tgcacctttt |
| 1441 | caccgccaaa gaggtgtcct tctccaacaa gccctacttc cggggtgagt tctgcagtca |
| 1501 | gggtgtccgt gagggcctca tcgtgggctt cgtccactct atgtgccaga cggctcagag |
| 1561 | cttctgcgac agccctgggg agaagggggg tgccacacca cctgccctgc tcctgctgct |
| 1621 | ctcccgcctc tgcctggact acgagacggc caccatctcc tacatcctca ctctcactga |
| 1681 | tgaacagttt ctggtgcagg atcagttccc agtgacgccc gtgagcacgc tgtgtgcaga |
| 1741 | ggccagggaa acggcgcggc ggctgctgac ccactacgtg aaggtgcagg gcctggtcat |
| 1801 | atcacagatg ctgcgcaaga gcgtggagac tcgcgactgg ctcagcactc tggagccccg |
| 1861 | gaatgtgcgg gccgtcatga agcgggtggt ggaggatacc accgccatcg acgtgcaggt |
| 1921 | ggggctcctg tacgaagagg gtgttcgcaa ggcccagagc agcgactcca gcaagaggac |
| 1981 | tttctccgtg tacagcagct ctcggcagca gggccgctac gcccccagct atacccccag |
| 2041 | tgccccgatg gacaccaacc tcttgagcaa tatccagaag ctattctctg aacgtattga |
| 2101 | tgtgttcagc cctgtggagt tcaacaaggt gtcggtgctg accggcatca tcaagatcag |
| 2161 | cctgaagacg ctgctggagt gtgtgcggct gcgcaccttt gggcgcttcg ggctgcagca |
| 2221 | ggtgcaagtg gactgccact ttctgcagct ctacctgtgg cgttttgtgg ccgacgaaga |
| 2281 | actcgtgcac ttgctgctgg acgaagtggt ggcctctgct gccctgcgct gcccagaccc |
| 2341 | tgtgcccatg gagcccagtg tggttgaggt catctgcgag cgcggctagg cgcagccgct |
| 2401 | gccatgcacc ggtctgtccc tgcaccccat ggcacccagg atctggtctc ggtggtcctt |
| 2461 | ccccgcaggc aggtgtcagg accggcctaa taaacatgtg tggcctcctc aaaaaaaaaa |
| 2521 | aaaaaaaaaa aaaaaaaaaa aaaaaa |

An exemplary human PDGFA amino acid sequence is set forth below (SEQ ID NO: 63; GenBank Accession No: P04085.1, Version 1, incorporated herein by reference):

| | |
|---|---|
| 1 | mrtlacllll gcgylahvla eeaeiprevi erlarsqihs irdlqrllei dsvgsedsld |
| 61 | tslrahgvha tkhvpekrpl pirrkrsiee avpavcktrt viyeiprsqv dptsanfliw |
| 121 | ppcvevkrct gccntssvkc qpsrvhhrsv kvakveyvrk kpklkevqvr leehlecaca |
| 181 | ttslnpdyre edtgrpresg kkrkrkrlkp t |

An exemplary human PDGFA nucleic acid sequence is set forth below (SEQ ID NO: 64; GenBank Accession No: AH002927.2, Version 2, incorporated herein by reference):

| | |
|---|---|
| 1 | ttcgctccca cccggtgccg cagattgcag ctggcactgg agggtgggca agctcgaggg |
| 61 | aggggcgcgg agcgcgcgga cgcgcgcggg gctttgatgg atttagctgc ttgcgcgagc |
| 121 | gcgtgtgtgc tccctgccgc ggcggcgccc gggccctgcc gggtccgcac gaaccccgag |
| 181 | cgcttccgag gtgcgggtcc caggcccgga atccggggag gcgggggggg gggcgggggc |
| 241 | gggggcgggg gaggggcgcg gcggcggcgg ctataaccct ctccccgccg ccggccggct |
| 301 | ccacacgcgc gccctgcgga gcccgcccaa ctccggcgag ccggcctgcg cctactcctc |
| 361 | ctcctcctct cccggcggcg gctgcggcgg aggcgcgact cgccttcgcc cgccctcagg |
| 421 | cccgcgcggg cggcgcagcg aggccccggg cggcgggtgg tggctgccag cggcgtcggc |
| 481 | cggccgctgc ccggccccgg cgagcggagg gcggagcgcg gcgccggagc cgagggccgc |
| 541 | cgcggagggg gtgctgggcc gcgctgtgcc cggccgggcg gcggctgcaa gaggaggccg |
| 601 | gaggcgagcg cggggccggc ggtgggcgcg cagggcggct cgcagctcgc agccggggcc |
| 661 | gggccaggcg ttcaggcagg tgatcggtgt ggcggcggcg gcggcggcgg ccccagactc |
| 721 | cctccggagt tcttcttcgg ggctcgatgt ccgcaaatat gcagaattac cggccgggtc |
| 781 | gctcctgaag ccagcgcggg gagcgagcgc ggcggcggcc agcaccggga acgcaccgag |
| 841 | gaagaagccc agcccccgcc ctccgcccct tccgtcccca ccccctaccc ggcggcccag |
| 901 | gaggctcccc gcgctcgcgg cgcgcactcc ctgtttctcc tcctcctggc tggcgctgcc |
| 961 | tgcctctccg cactcactgc tcgccgggcg ccgtccgcca gctccgtgct ccccgcgcca |
| 1021 | ccctcctccg ggccgcgctc cctaagggat ggtactgaat ttcgccgcca caggagaccg |
| 1081 | gctggagcgc cgccccgcgg cctcgcctct cctccgagca gccagcgcct cgggacgcga |
| 1141 | tgaggacctt ggcttgcctg ctgctcctcg gctgcggata cctcgcccat gttctggccg |
| 1201 | aggttggtgc cgcccccgcg ccccgtctca cgctcggctc ctccggcgca caccccccgc |
| 1261 | cggctggggc ccacgggctc tgcagnnnnn nnnnnnnnnn nnnnnnnnnn nnnnnnnnnn |
| 1321 | nnnnnnnnnn nnnnnnnnnn nnnnnnnnnn nnnnnnnnnn nnnnnnnnnn nnnnnnnnnn |
| 1381 | nnnnntgacc gtgtggcctc tgcttgcagg aagccgagat cccccgcgag gtgatcgaga |
| 1441 | ggctggcccg cagtcagatc cacagcatcc gggacctcca gcgactcctg gagatagact |
| 1501 | ccgtaggtaa atcgcgcccc ttccctcgcg cgcgggnnnn nnnnnnnnnn nnnnnnnnnn |
| 1561 | nnnnnnnnnn nnnnnnnnnn nnnnnnnnnn nnnnnnnnnn nnnnnnnnnn nnnnnnnnnn |
| 1621 | nnnnnnnnnn nnnnnnaggg cccctaatgg cgggagctgt gaggggctgt gccgccaggt |
| 1681 | gcctgttccc cagtggctcc caaagctggt ctgtgggaag tgcggctgga caggcccagg |
| 1741 | gcacagcgca cggggcattc acggtgttct ccttccgcct gcagggagtg aggattcttt |
| 1801 | ggacaccagc ctgacacgtg acggggtcca tgccactaag catgtgcccg agaagaggcc |
| 1861 | cctgcccatt cggaggaaga gaagcatcgg tgagtccagg aggccgcgat gggcagggca |
| 1921 | gggccgggtc ggggtgagtc caggaggccg cgatgggcag ggcagggccg ggtggggagg |
| 1981 | aggagctgcc cgctctccca gcgcagtggc ctcatggcaa gccacccgtt ccctcctccc |
| 2041 | taaaataggc ctggccctgg tgcctctggc tctggcctct ccgagggtgt ctcctgcccn |
| 2101 | nnnnnnnnnn nnnnnnnnnn nnnnnnnnnn nnnnnnnnnn nnnnnnnnnn nnnnnnnnnn |
| 2161 | nnnnnnnnnn nnnnnnnnnn nnnnnnnnnn nnnnnnnnnt gctcctgccc acccagcccc |
| 2221 | tgagcctctg ctcccagctc agcctctgct gcctgggagg aatcctggcc tgtgggttac |
| 2281 | cctggttgcc ccccaggccc agctggagcc gcctcagccc tggggtgggg ccgtggtcgc |
| 2341 | agaggccggt ccccgctcac tgtgcccccg ccgttgcaga ggaagctgtc cccgctgtct |
| 2401 | gcaagaccag gacggtcatt tacgagattc ctcggagtca ggtcgacccc acgtccgcca |
| 2461 | acttcctgat ctggcccccg tgcgtggagg tgaaacgctg caccggctgc tgcaacacga |
| 2521 | gcagtgtcaa gtgccagccc tcccgcgtcc accaccgcag cgtcaaggtg agcnnnnnnn |
| 2581 | nnnnnnnnnn nnnnnnnnnn nnnnnnnnnn nnnnnnnnnn nnnnnnnnnn nnnnnnnnnn |
| 2641 | nnnnnnnnnn nnnnnnnnnn nnnnnnnnnn nnnctgcagc ttgtaggttt tcacctggta |
| 2701 | ctgctacact ccccaccaca aggtagtgtt tctgggagga ggtcaggggt caggctgctc |
| 2761 | tctcctccct ctcagcctgt ccccggctcc cagcacgtcg tgatgtccga agctccatgc |
| 2821 | aggcattcat ggccgggctc tgttctctct ggcaggtggc caaggtggaa tacgtcagga |
| 2881 | agaagccaaa attaaaagaa gtccaggtga ggttagagga gcatttggag tgcgcctgcg |
| 2941 | cgaccacaag cctgaatccg gattatcggg aagaggacac gggtgagtgg ctgccttcgt |
| 3001 | cggcatcgtg ttggagaaca ggtcttcaga gccttgcttt tggggtgtta ggtggccccc |
| 3061 | ttgagcgcaa cgcttactgc tgtgagcatc tgggctgctg ttgaaggatt cgttgccctg |
| 3121 | ctcccgggcc agatgcctgc gggggagacg gatccnnnnn nnnnnnnnnn nnnnnnnnnn |
| 3181 | nnnnnnnnnn nnnnnnnnnn nnnnnnnnnn nnnnnnnnnn nnnnnnnnnn nnnnnnnnnn |
| 3241 | nnnnnnnnnn nnnnntctcg gtgccagggt gctggccttc tcttctggaa agataaagaa |
| 3301 | atgccgtagg tatttgttgc ttcagttctt caccccgacg gccgtccctc ggcccactca |
| 3361 | ccgccctgcc cttttgttaa caggaaggcc tagggagtca ggtaaaaaac ggaaaagaaa |
| 3421 | aaggttaaaa cccacctaaa gcagccaacc aggtaggact gtctgccgga cactgagtcc |
| 3481 | tgctaggcat gcaagnnnnn nnnnnnnnnn nnnnnnnnnn nnnnnnnnnn nnnnnnnnnn |
| 3541 | nnnnnnnnnn nnnnnnnnnn nnnnnnnnnn nnnnnnnnnn nnnnnnnnnn nnnnnctgca |
| 3601 | gatgtgaggt gaggatgagc cgcagccctt tcctgggaca tggatgtaca tggcgtgtta |
| 3661 | cattcctgaa cctactatgt acggtgcttt attgccagtg gcggtctttg ttctcctccg |
| 3721 | tgaaaaactg tgtccgagaa cactcgggag aacaaagaga cagtgcacat ttgtttaatg |
| 3781 | tgacatcaag caagtattgt agcactcggt gaagcagtaa gaagcttcct tgtcaaaaag |
| 3841 | agagagagag agagagagag agaaaacaaa accacaaatg acaaaaacaa aacggactca |
| 3901 | caaaaatatc taaactcgat gagatggagg gtcgccccgt gggatggaag tgcagaggtc |
| 3961 | tcagcagact ggatttctgt ccgggtggtc acagctgctt ttttgccgag gatgcagagc |
| 4021 | ctgctttggg aacgactcca gaggggtgcg tggtgggctc tgcaggggcc cgcaggaagc |
| 4081 | aggaatgtct tggaaaccgc cacggaaact ttagaaacca cacctcctcg ctgtagtatt |
| 4141 | taagcccata cagaaacctt cctgagagcc ttaagtggtt tttttttttg tttttgtttt |
| 4201 | gttttttttt tttttgtttt tttttttttt tttttacacc ataaagtgat tattaagctt |
| 4261 | tttactcttt tgctagcttt tttttttttt ttttttaatt atctctcgga tgacattcac |
| 4321 | acccacaaca cacaggctgc tgtaactgtc aggacagtgc gacggtattt ttcctagcaa |
| 4381 | gatgcaaact aatgagatgt attaaaataa acatggtata cctcctatgc atcatttcct |
| 4441 | aaatctttct ggctttgtgt ttctccctta ccctgcttta tttcttaatt taagccattt |
| 4501 | tgaagaacta tgcgtcaacc aatcgtacgc ctcctgcggc actgcccaga gccc |

An exemplary human PCDHB2 amino acid sequence is set forth below (SEQ ID NO: 65; GenBank Accession No: EAW61983.1, Version 1, incorporated herein by reference):

| | |
|---|---|
| 1 | meagegkerv pkqrqvliff vllgiaqasc qprhysvaee tesgsfvanl lkdlgleige |
| 61 | lavrgarvvs kgkkmhlqfd rqtgdlllne kldreelcgp tepcvlpfqv llenplqffq |
| 121 | aelrirdvnd hspvfldkei llkipesitp gttflieraq dldvgtnslq nytispnfhf |
| 181 | hlnlqdsldg iilpqlvinr aldreeqpei rltltaldgg spprsgtalv rievvdindn |
| 241 | vpefakllye vqipedspvg sqvaivsard ldigtngeis yafsqasedi rktfrlsaks |
| 301 | gelllrqkld fesiqtytvn iqatdgggls gtcvvfvqvm dlndnppelt mstlinqipe |
| 361 | nlqdtliavf svsdpdsgdn grmvcsiqdd lpfflkpsve nfytlvista ldretrseyn |
| 421 | ititvtdfgt prlktehnit vlvsdvndna paftqtsytl fvrennspal higsvsatdr |
| 481 | dsgtnaqvty sllppqdphl plaslvsina dnghlfalqs ldyealqafe frvgaadrgs |
| 541 | palssealvr vlvldandns pfvlyplqng sapctelvpr aaepgylvtk vvavdgdsgq |
| 601 | nawlsyqllk atepglfgvw ahngevrtar llrerdaakq rlvvlvkdng epprsatatl |
| 661 | hvllvdgfsq pylllpeaap aqaqadlltv ylvvalasvs slflfsvllf vavrlcrrsr |
| 721 | aasvgrcsvp egpfpgqmvd vsgtgtlsqs yqyevcltgg sgtnefkflk piipnfvaqg |
| 781 | aervseanps frksfeft |

An exemplary human PCDHB2 nucleic acid sequence is set forth below (SEQ ID NO: 66; GenBank Accession No: NM_018936.3, Version 3, incorporated herein by reference):

| | |
|---|---|
| 1 | ccttacccag atactcagct aaagaagcag caagcaggaa gaggaggctt tctaaggcgg |
| 61 | tcgctccggg aaatccgggc cctaggattg tccactcatc ccagtatcag cgagatacgg |
| 121 | ggagatagag ttagcgacaa cgtgagccag agctggagca cgtttggtga gagaccagaa |
| 181 | agcaatggag gccggagagg ggaaggagcg cgttccgaaa caaaggcaag tcctgatatt |
| 241 | ctttgttttg ctgggcatag ctcaggctag ttgccagcct aggcactatt cagtggccga |
| 301 | ggaaacggag agtggctcct ttgtggccaa tttgttaaaa gacctggggc tggagatagg |
| 361 | agaacttgct gtgagggggg ccagggtcgt ttccaaagga aaaaaaatgc atttgcagtt |
| 421 | cgataggcag accggggatt tgttgttaaa tgagaaattg gaccgggagg agctgtgcgg |
| 481 | ccccacagag ccctgtgtcc tacctttcca ggtgttacta gaaaatccct tgcagttttt |
| 541 | tcaggcggag ctacggatta gggacgtaaa tgatcattcc ccagttttcc tagacaaaga |
| 601 | aatacttttg aaaattccag aaagtatcac tcctggaact actttcttaa tagaacgtgc |
| 661 | ccaggacttg gatgtaggaa ccaacagtct ccaaaattac acaatcagtc ccaatttcca |
| 721 | ctttcatctt aatttacaag acagtctcga tggcataata ttaccacagc tggtgctgaa |
| 781 | cagagccctg gatcgcgagg agcagcctga gatcaggtta accctcacag cgctagatgg |
| 841 | cgggagtcca cccaggtccg gcacggccct ggtacggatt gaagttgtgg acatcaatga |
| 901 | caacgtccca gagtttgcaa agctgctcta tgaggtgcag atcccggagg acagccccgt |
| 961 | tggatcccag gttgccatcg tctctgccag ggatttagac attggaacta atggagaaat |
| 1021 | atcttatgca ttttcccaag catctgaaga cattcgcaaa acgtttcgat taagtgcaaa |
| 1081 | atcgggagaa ctgcttttaa gacagaaact ggatttcgaa tccatccaga catacacagt |
| 1141 | aaatattcag gcgacagatg gtgggggcct atctggaact tgtgtggtat ttgtccaagt |
| 1201 | gatggatttg aatgacaatc ctccggaact aactatgtcg acacttatca atcagatccc |
| 1261 | agaaaacttg caggacaccc tcattgctgt attcagcgtt tcagatcctg actccggaga |
| 1321 | caacggaagg atggtgtgct ccatccaaga tgatcttcct tttttcttga aaccttctgt |
| 1381 | tgagaacttt tacactctgg tgataagcac ggccctggac cgggagacca gatccgaata |
| 1441 | caacatcacc atcaccgtca ccgacttcgg gacacccagg ctgaaaaccg agcacaacat |
| 1501 | aaccgtgctg gtctccgacg tcaatgacaa cgcccccgcc ttcacccaaa cctcctacac |
| 1561 | cctgttcgtc cgcgagaaca acagccccgc cctgcacatc ggcagcgtca gcgccacaga |
| 1621 | cagagactcg ggcaccaacg cccaggtcac ctactcgctg ctgccgcccc aggacccgca |
| 1681 | cctgcccctc gcctccctgg tctccatcaa cgcggacaac ggccacctgt tcgctctcca |
| 1741 | gtcgctggac tacgaggccc tgcaggcgtt cgagttccgc gtgggcgccg cagaccgcgg |
| 1801 | ctccccggcg ttgagcagcg aggcgctggt gcgcgtgctg gtgctggacg ccaacgacaa |
| 1861 | ctcgcccttc gtgctgtacc cgctgcagaa cggctccgcg ccctgcaccg agctggtgcc |
| 1921 | ccgggcggcc gagccgggct acctggtgac caaggtggtg gcggtggacg gcgactcggg |
| 1981 | ccagaacgcc tggctgtcgt accagctgct caaggccacg gagcccgggc tgttcggcgt |
| 2041 | gtgggcgcac aatggcgagg tgcgcaccgc caggctgctg agggagcgcg acgctgccaa |
| 2101 | gcagaggctg gtggtgctgg tcaaggacaa tggcgagcct ccgcgctcgg ccaccgccac |
| 2161 | gctgcacgtg ctcctggtgg acggcttctc ccagccctac ctgctgctcc cggaggcggc |
| 2221 | accggcccag gcccaggccg acttgctcac cgtctacctg gtggtggcgt tggcctcggt |
| 2281 | gtcttcgctc ttcctcttct cggtgctcct gttcgtggcg gtgcggctgt gcaggaggag |
| 2341 | cagggcggcc tcggtgggtc gctgctcggt gcccgagggc ccctttccag ggcagatggt |
| 2401 | ggacgtgagc ggcaccggga ccctgtccca gagctaccag tacgaggtgt gtctgactgg |
| 2461 | aggctccggg acaaatgagt tcaagttcct gaagccaatt atccccaact tcgttgctca |
| 2521 | gggtgcagag agggttagcg aggcaaatcc cagtttcagg aagagctttg aattcactta |
| 2581 | agtgttaata aggatctact gaggctagtc tcgtttaatt tgtggaaagt ccttttttac |
| 2641 | tgctttgccc attggaggtg tctcctttta ttagaaagta accatcttat tccaattcta |
| 2701 | tgcatgttac tggtatttat aaatgtatga gtttttttgc ggtataataa atgtaaattt |
| 2761 | tctttgtatt ctaaaaaaa |

An exemplary human PCDHB3 amino acid sequence is set forth below (SEQ ID NO: 67; GenBank Accession No: EAW61981.1, Version 1, incorporated herein by reference):

| | |
|---|---|
| 1 | meaggerflr qrqvlllfvf lggslagses rrysvaeeke kgflianlak dlglrveela |
| 61 | argaqvvskg nkqhfqlshq tgdlllnekl dreelcgpte pcilhfqill qnplqfvtne |
| 121 | lriidvndhs pvffenemhl kilestlpgt viplgnaedl dvgrnslqny titpnshfhv |
| 181 | ltrsrrdgrk ypelvldkal dreeqpelsl tltaldggsp prsgtaqini qvldindnap |
| 241 | efaqplyeva vlentpvnsv ivtvsasdld tgsfgtisya ffhaseeirk tfqlnpitgd |
| 301 | mqlvkylnfe ainsyevdie akdggglsgk stvivqvvdv ndnppeltls svnspipens |
| 361 | getvlavfsv sdldsgdngr vmcsiennlp fflkpsvenf ytlvsegald retrseynit |
| 421 | ititdlgtpr lktkynitvl vsdvndnapa ftqisytlfv rennspalhi gsvsatdrds |
| 481 | gtnaqvtysl lppqdphlpl sslvsinadn ghlfalrsld yealqafefr vgatdrgspa |
| 541 | lssealvrvl vldandnspf vlyplqngsa pctelvpraa epgylvtkvv avdgdsgqna |
| 601 | wlsyqllkat epglfgvwah ngevrtarll serdaakhrl vvlvkdngep prsatatlhv |
| 661 | llvdgfsqpy lplpeaapaq aqadlltvyl vvalasvssl flfsvllfva vrlcrrsraa |
| 721 | svgrcsvpeg pfpgqmvdvs gtgtlsqsyq yevcltggsg tnefkflkpi ipnfvaqgae |
| 781 | rvseanpsfr ksfefs |

An exemplary human PCDHB3 nucleic acid sequence is set forth below (SEQ ID NO: 68; GenBank Accession No: NM_018937.4, Version 4, incorporated herein by reference):

| | |
|---|---|
| 1 | tgctcgtaga taaaagtgca ttttatttcc ctagattgca tttatttaat tcatataaca |
| 61 | tgagaaactc ctccagtagc gtcaactagg gttgataaga ataatcgata aagcaaaata |
| 121 | aaaacacctt ctccaagatt ttgtaactgc aagcgaacgc atggtggcgc tgttgactaa |
| 181 | gaaggcgaat taaaccacag gcattgtgca tgctcggtga cgcacggatc cagtgtggta |
| 241 | aaccagcggt tgagagccca ggcagatttt tgagccagca agtctgagcc tctggaaagg |
| 301 | cttattcact aggccgtcta caaaggttgt ggggcaaaag actgtttccc agctctgtct |
| 361 | gaggttcagc ttggcgacat tccctggaag agcgtgacgg aaagtgcaat ggaggcggga |
| 421 | ggagagcgat ttcttagaca aaggcaagtc ttgcttctct ttgtttttct gggagggtct |
| 481 | ctggctgggt ccgagtcaag acgctattct gtggctgagg aaaaagagaa gggcttttta |
| 541 | atagccaacc tagcaaagga tctgggacta agggtagagg aactggccgc gaggggggcc |
| 601 | caagttgtgt ccaaagggaa caaacagcat tttcagctca gtcatcagac aggtgatttg |
| 661 | ctcctgaatg agaaattgga ccgggaggag ctatgcggcc ccacagaacc atgcatacta |
| 721 | cattttcaga tattactgca aaaccctttg caattcgtta caaacgagct ccgtatcata |
| 781 | gatgtaaatg accattctcc ggtattcttt gaaaatgaaa tgcatctgaa aatcctagaa |
| 841 | agcactctgc caggaacagt aattcctttg ggaaatgctg aggacttgga tgtgggaaga |
| 901 | aacagcctcc aaaactacac tatcactccg aattcccact tccacgtact cactcgcagt |
| 961 | cgtagggacg gaaggaagta cccggaacta gtactggata aagcgctcga tcgggaggag |
| 1021 | cagccggaac tcagcttaac gctcaccgcg ctggacggcg gctctccccc tcggtctggg |
| 1081 | acagcccaga taaacatcca ggtcttagat ataaacgaca atgcaccaga atttgcacag |
| 1141 | ccgctctatg aggttgcagt tctagagaat acccccgtta actctgtcat tgtcactgtc |
| 1201 | tcggcttctg acttagatac aggaagtttt gggacaatat catatgcatt ttttcatgct |
| 1261 | tctgaagaaa ttcgcaaaac ttttcagcta aatccaatta ctggtgatat gcaactggtc |
| 1321 | aaatatttga attttgaagc gattaatagt tatgaagtcg acatcgaggc caaggatggc |
| 1381 | ggaggcctat ccggaaagtc tacagtcata gtccaggtgg ttgatgtcaa cgacaaccca |
| 1441 | ccggaactga ccttgtcttc agtaaacagc cctattcctg agaactcggg agagactgta |
| 1501 | ctggctgttt tcagtgtttc tgatctagac tctggagaca acggaagagt gatgtgttcc |
| 1561 | attgagaaca atctcccctt cttcctgaaa ccatctgtag agaattttta caccctagtg |
| 1621 | tcagaaggcg cgctggacag agagaccaga tccgagtaca acattaccat cactatcact |
| 1681 | gacctgggga cacccaggct gaaaaccaag tacaacataa ccgtgctggt ctccgacgtc |
| 1741 | aatgacaacg cccccgcctt cacccaaatc tcctacaccc tgttcgtccg cgagaacaac |
| 1801 | agccccgccc tgcacatcgg cagtgtcagc gccacagaca gagactcagg caccaacgcc |
| 1861 | caggtaacct actcgctgct gccgccccag gacccgcacc tgcccctctc ttccctggtc |
| 1921 | tccatcaacg cggacaacgg ccacctgttt gccctcaggt cgctggacta cgaggccctg |
| 1981 | caggcgttcg agttccgcgt gggcgccaca gaccgtggct ccccggcttt gagcagcgag |
| 2041 | gcgctggtgc gcgtgctggt gctggacgcc aacgacaact cgcccttcgt gctgtacccg |
| 2101 | ctgcagaacg gctccgcgcc ctgcaccgag ctggtgcccc gggcggctga gccgggctac |
| 2161 | ctggtgacca aggtggtggc ggtggacggc gactcgggcc agaacgcctg gctgtcgtac |
| 2221 | cagctgctca aggccacgga gcccgggctg ttcggcgtgt gggcgcacaa tggcgaagtg |
| 2281 | cgcaccgcca ggctgctgag cgagcgcgac gcggccaagc acaggctggt ggtgctggtc |
| 2341 | aaggacaatg gcgagcctcc gcgctcggcc accgccacgc tgcatgtgct cctggtggac |
| 2401 | ggcttctccc agccctacct gcctctcccg gaggcggcac cggcccaggc ccaggccgac |
| 2461 | ttgctcaccg tctacctggt ggtggcattg gcctcggtgt cttcgctctt cctcttttcg |
| 2521 | gtgctcctgt tcgtggcggt gcggctgtgc aggaggagca gggcggcctc ggtgggtcgc |
| 2581 | tgctcggtgc ccgagggccc ctttccaggg cagatggtgg acgtgagcgg caccgggacc |
| 2641 | ctgtcccaga gctaccagta cgaggtgtgt ctgactggag gctccgggac aaatgagttc |
| 2701 | aagttcctga agccaattat ccccaacttc gttgctcagg gtgcagagag ggttagcgag |
| 2761 | gcaaatccca gtttcaggaa gagctttgaa ttcagttaag tgttaataag gatctactga |
| 2821 | gcctcgtctt agttaatctg tggaaagtcc ttttttactg ctttgtccat tggagaggtc |
| 2881 | ttttttggtc tggttcaagg caagtagcaa gaatagagca aaatatcaaa tccagggatg |
| 2941 | gcttaggttt cattaacagt actggaaagt agttgtgtgg ctctgaatgt tttgtatttc |
| 3001 | aatcgagaat ccttagtcga tagaacattt tgtttatata ttgattctac tttttctgta |
| 3061 | gttaatcctt gcatattctc ctttcatcct ggcttgccaa cgcagtctta attccgcctt |
| 3121 | ttttttttct aatggggagc aaaaagaaat tcactgtctt ttaatagtga tttcaaatag |
| 3181 | cttattaaaa taactccatt caaattttac attataaagc aatgtagaga gagttccaaa |
| 3241 | ccaccaattt tataatttcc cttgttgaat atattcatat aatgtgttct ataatatgcc |
| 3301 | caaagcagct ttgtctatag ttaacaaagt tttaaggata gacaagaatg tgttttcttt |
| 3361 | aataaatagt aatatatcat ctttttaggg atatagtact caaatgaaag taatttagtt |
| 3421 | cattttctgt gttgacattt gcaattaata tttcaatatt ttatgtgctt atattggcca |
| 3481 | aaatatggac acaaatatag actaatatgg gtaattaccc tttggtttat ctaaagtgtg |
| 3541 | ttcatgatga ctgaggaaaa aaattaaacc tatgccattt aaaaaaaaaa aaa |

An exemplary human PCDHB6 amino acid sequence is set forth below (SEQ ID NO: 69; GenBank Accession No: EAW61978.1, Version 1, incorporated herein by reference):

| | |
|---|---|
| 1 | mmqtkvqnkk rqvaffillm lwgevgsesi qysvleetes gtfvanltkd lglrvgelas |
| 61 | rgarvvfkgn rqhlqfdpqt hdlllnekld reelcgstep cvlpfqvlle nplqffqasl |
| 121 | rvrdindhap efparemllk iseitmpgki fplkmandld tgsnglqryt issnphfhvl |
| 181 | trnrsegrkf pelvldkpld reeqpqlrlt lialdggspp rsgtseiqiq vldindnvpe |
| 241 | faqelyeaqv pennplgslv itvsardlda gsfgkvsyal fqvddvnqpf einaitgeir |
| 301 | lrkaldfeei qsydvdveat dggglsgkcs lvvrvldvnd napeltmsff islipenlpe |
| 361 | itvavfsvsd adsghnqqvi csiennlpfl lrpsvenfyt lvtegaldre sraeynitit |
| 421 | vtdlgtprlk tqqsitvqvs dvndnapaft qtsytlfvre nnspalhigs vsatdrdsgi |
| 481 | naqvtysllp pqdphlplss lvsinadngh lfalrsldye alqsfefrvg atdrgspals |
| 541 | sealvrllvl dandnspfvl yplqngsapc telvpraaep gylvtkvvav dgdsgqnawl |
| 601 | syqllkatep glfgvwahng evrtarllse rdaakqrlvv lvkdngeppr satatlhvll |
| 661 | vdgfsqpylp lpeaapaqaq adsltvylvv alasvsslfl fsvllfvavr lcrrsraasv |
| 721 | grysvpegpf pghlvdvsgt gtlsqsyqyk vcltggsetn efkflkpimp nfppqgtere |
| 781 | meetptsrns fpfs |

An exemplary human PCDHB6 nucleic acid sequence is set forth below (SEQ ID NO: 70; GenBank Accession No: AF217752.1, Version 1, incorporated herein by reference):

| | |
|---|---|
| 1 | atgatgcaaa ctaaagtaca gaacaagaaa aggcaagtgg ctttcttcat tttattgatg |
| 61 | ctttggggag aggtgggttc tgaatcgatt cagtattccg tattggagga gacagaaagt |
| 121 | ggcacgtttg tggccaactt gacaaaggac ctgggactga gggtggggga gctggcttcg |
| 181 | cggggcgctc gggttgtttt caaagggaac agacaacatt tgcagtttga tccacagacc |
| 241 | catgatttac tgctaaatga aaaactggac cgggaggagc tgtgtggctc cactgagccg |
| 301 | tgtgtgctac ctttccaagt gttactggaa aaccccttgc agttttttca ggcttccttg |
| 361 | cgagtcagag atataaatga ccacgccccg gaattccctg ccagagaaat gctcctgaaa |
| 421 | atatcagaaa ttactatgcc aggaaagata tttcctttga aaatggcaca cgatttagac |
| 481 | accggcagca acggccttca gaggtacaca atcagctcca accctcactt ccacgttctc |
| 541 | acccgcaatc gcagcgaagg caggaagttc ccggagctgg tgctagacaa accgttggac |
| 601 | cgcgaggagc agccccaact caggctaacg ctgatcgcgc tggatggcgg gtctccgccc |
| 661 | cggtcaggga cctccgagat tcagatccag gttttggaca tcaatgacaa cgtccccgag |
| 721 | tttgctcagg agctctatga agcacaagtc cctgagaaca accccctcgg ctctctggtt |
| 781 | attaccgtct cagccagaga tttagatgca ggatcgtttg ggaaggtatc ttacgccctg |
| 841 | tttcaagtcg atgacgtcaa ccaacccttc gaaataaacg caatcacagg agaaattcgg |
| 901 | ctgagaaagg ctttggattt tgaggaaatt cagtcttatg acgtggatgt tgaggctaca |
| 961 | gatggtggag gcctatcagg aaaatgctct ttagtcgtca gggtcctgga cgtgaatgac |
| 1021 | aatgcccctg aactcaccat gtcgttcttc atcagcctca tcccagaaaa cttaccagag |
| 1081 | atcacagtgg cagttttcag tgtttcagat gcagactctg gacataacca acaggttatt |
| 1141 | tgttcaatag agaacaatct cccctttcta ctaagacctt ccgtggagaa tttctacacc |
| 1201 | ctggtaacag aaggcgcgct ggacagagag agcagagccg agtacaacat cactatcacg |
| 1261 | gtcactgatt tggggacacc aaggctgaaa acccagcaga gcataactgt gcaggtctcc |
| 1321 | gacgtcaatg acaacgtccc cgccttcacc caaacctcct acaccctgtt cgtccgcgag |
| 1381 | aacaacagcc ccgccctgca catcggcagc gtcagcgcca cagacagaga ctcaggcatc |
| 1441 | aacgcccagg tcacctactc gctgctgccg ccccaggacc cgcacctgcc cctctcttcc |
| 1501 | ctggtctcca tcaacgcgga caacggccac ctgtttgccc tcaggtcgct ggactacgag |
| 1561 | gccctgcagt ctttcgagtt ccgcgtgggc gccacagacc gcggctcccc ggcgttgagc |
| 1621 | agcgaggcgc tggtgcgctt gctggtgctg gacgccaacg acaactcgcc cttcgtgttg |
| 1681 | tacccgctgc agaacggctc cgcgccctgc accgagctgg tgccccgggc ggccgagccg |
| 1741 | ggctacctgg tgaccaaggt ggtggcggtg gacggcgact cgggccagaa cgcctggctg |
| 1801 | tcgtaccagc tgctcaaggc cacggagctc ggtctgttcg gcgtgtgggc gcacaatggc |
| 1861 | gaggtgcgca ccgccaggct gctgagcgag cgagacgcag ccaagcacag gctggtggtg |
| 1921 | cttgtcaagg acaatggcga gcctccgcgc tcggccaccg ccacgctgca cgtgctcctg |
| 1981 | gtggacggct tctcccagcc ctacctgcct ctccctgagg cggccccggc ccaagcccag |
| 2041 | gccgactctc tcaccgtcta cctggtggtg gcgttggcct cggtgtcgtc gctcttcctc |
| 2101 | ttttcggtgc tcctgttcgt ggcggtgcgg ctgtgcagga ggagcagggc ggcctcggtg |
| 2161 | ggtcgctact cggtgcccga gggtcccttt ccagggcatc tggtggatgt gagcggcacc |
| 2221 | gggaccctat cccagagcta ccagtacaag gtgtgtctga cgggaggctc agaaacaaat |
| 2281 | gagttcaagt tcctgaagcc gattatgccc aacttccctc ctcagggcac tgagagagaa |
| 2341 | atggaagaaa cccccacctc tcggaatagc ttcccgttca gttaagtgtg ggattatttt |
| 2401 | actaaatctt acttatgttt ggagatctct tttaacttaa agttacatgg tctgtttctt |
| 2461 | gtttatttta cctctattct ttaggttgaa attttatata aagtaagata ctggtatctt |
| 2521 | agtatttcct gttcatgctt agtagtttat tacttcactt gagggtactt gacaatatga |
| 2581 | acaaaaagta aatttttatt tgcataattt taagcttttg aaattaaatt atctattctt |
| 2641 | ccccccccca aaaaaaagta ttgtaaatcc ttaagtaaaa ttgtatttct agctattggt |
| 2701 | aagagttgtt tcactattgc tatgtaggac tgtttaaaat gtgagtatct gatattattt |
| 2761 | aatcctccaa tgtctcattt tgcagtaact cctacagtgt gtaacactaa aaataagaac |
| 2821 | taatgatggc taaacactaa agtagccatt catacttatg catattttag tatcccataa |
| 2881 | tagtcaatcc aaaatttttg tgactataga ctttactgaa gtgtcaacac attagtttgt |
| 2941 | gagcctcatg taagaacatg atggtctttt tttaaaaaaa aagtcgtgcc aattataagt |
| 3001 | gcttaataaa tatttgctga atgttactaa |

An exemplary human PCDHB10 amino acid sequence is set forth below (SEQ ID NO: 71; GenBank Accession No: AAQ89082.1, Version 1, incorporated herein by reference):

| | |
|---|---|
| 1 | mavrelcfpr qrqvlflflf wgvslagsgf grysvteete kgsfvvnlak dlglaegela |
| 61 | argtrvvsdd nkqyllldsh tgnlltnekl dreklcgpke pcmlyfqilm ddpfqiyrae |
| 121 | lrvrdindha pvfqdketvl kisentaegt afrleraqdp dgglngiqny tispnsffhi |
| 181 | nisggdegmi ypelvldkal dreeqgelsl tltaldggsp srsgtstvri vvldvndnap |
| 241 | qfaqalyetq apenspigfl ivkvwaedvd sgvnaevsys ffdasenirt tfqinpfsge |
| 301 | iflrelldye lvnsykiniq amdggglsar crvlvevldt ndnppelivs sfsnsvaens |
| 361 | petplavfki ndrdsgengk mvcyiqenlp fllkpsvenf yilitegald reiraeynit |
| 421 | itvtdlgtpr lktehnitvl vsdvndnapa ftqtsytlfv rennspalhi gsvsatdrds |
| 481 | gtnaqvtysl lppqdphlpl aslvsinadn ghlfalrsld yealqafefr vgatdrgspa |
| 541 | lsrealvrvl vldandnspf vlyplqngsa pctelvpraa epgylvtkvv avdgdsgqna |
| 601 | wlsyqllkat epglfgvwah ngevrtarll serdaakhrl vvlvkdngep prsatatlhl |
| 661 | llvdgfsqpy lplpeaapaq aqaeadlltv ylvvalasvs slfllsvllf vavrlcrrsr |
| 721 | aasvgrcsvp egpfpghlvd vrgaetlsqs yqyevcltgg pgtsefkflk pvisdiqaqg |
| 781 | pgrkgeenst frnsfgfniq |

An exemplary human PCDHB10 nucleic acid sequence is set forth below (SEQ ID NO: 72; GenBank Accession No: NM_018930.3, Version 3, incorporated herein by reference):

| | |
|---|---|
| 1 | gaagacacgg acagatgaac ttaaaagaga agctttagct gccaaagatt gggaaaggga |
| 61 | aaggacaaaa aagacccctg ggctacacgg cgtaggtgca gggtttccta ctgctgttct |
| 121 | tttatgctgg gagctgtggc tgtaaccaac taggaaataa cgtatgcagc agctatggct |
| 181 | gtcagagagt tgtgcttccc aagacaaagg caagtcctgt ttctttttct tttttgggga |
| 241 | gtgtccttgg caggttctgg gtttggacgt tattcggtga ctgaggaaac agagaaagga |
| 301 | tcctttgtgg tcaatctggc aaaggatctg ggactagcag agggggagct ggctgcaagg |
| 361 | ggaaccaggg tggtttccga tgataacaaa caatacctgc tcctggattc acataccggg |
| 421 | aatttgctca caaatgagaa actggaccga gagaagctgt gtggccctaa agagccctgt |
| 481 | atgctgtatt tccaaatttt aatggatgat ccctttcaga tttaccgggc tgagctgaga |
| 541 | gtcagggata taaatgatca cgcgccagta tttcaggaca aagaaacagt cttaaaaata |
| 601 | tcagaaaata cagctgaagg gacagcattt agactagaaa gagcacagga tccagatgga |
| 661 | ggacttaacg gtatccaaaa ctacacgatc agccccaact cttttttcca tattaacatt |
| 721 | agtggcggtg atgaaggcat gatatatcca gagctagtgt tggacaaagc actggatcgg |
| 781 | gaggagcagg gagagctcag cttaaccctc acagcgctgg atggtgggtc tccatccagg |
| 841 | tctgggacct ctactgtacg catcgttgtc ttggacgtca atgacaatgc cccacagttt |
| 901 | gcccaggctc tgtatgagac ccaggctcca gaaaacagcc ccattgggtt ccttattgtt |
| 961 | aaggtatggg cagaagatgt agactctgga gtcaacgcgg aagtatccta ttcatttttt |
| 1021 | gatgcctcag aaaatattcg aacaaccttt caaatcaatc ctttttctgg ggaaatcttt |
| 1081 | ctcagagaat tgcttgatta tgagttagta aattcttaca aaataaatat acaggcaatg |
| 1141 | gacggtggag gcctttctgc aagatgtagg gttttagtgg aagtattgga caccaatgac |
| 1201 | aatccccctg aactgatcgt atcatcattt tccaactctg ttgctgagaa ttctcctgag |
| 1261 | acgccgctgg ctgtttttaa gattaatgac agagactctg gagaaaatgg aaagatggtt |
| 1321 | tgctacattc aagagaatct gccattccta ctaaaacctt ctgtggagaa tttttacatc |
| 1381 | ctaattacag aaggcgcgct ggacagagag atcagagccg agtacaacat cactatcacc |
| 1441 | gtcactgact tggggacacc caggctgaaa accgagcaca acataacggt cctggtctcc |
| 1501 | gacgtcaatg acaacgcccc cgccttcacc caaacctcct acaccctgtt cgtccgcgag |
| 1561 | aacaacagcc ccgccctgca catcggcagc gtcagcgcca cagacagaga ctcgggcacc |
| 1621 | aacgcccagg tcacctactc gctgctgccg ccccaagacc cgcacctgcc cctcgcctcc |
| 1681 | ctggtctcca tcaacgcgga caacggccac ctgttcgccc tcaggtcgct ggactacgag |
| 1741 | gccctgcagg ctttcgagtt ccgcgtgggc gccacagacc gcggctcccc cgcgctgagc |
| 1801 | agagaggcgc tggtgcgcgt gctggtgctg gacgccaacg acaactcgcc cttcgtgctg |
| 1861 | tacccgctgc agaacggctc cgcgccctgc accgagctgg tgccccgggc ggccgagccg |
| 1921 | ggctacctgg tgaccaaggt ggtggcggtg gacggcgact cgggccagaa cgcctggctg |
| 1981 | tcgtaccagc tgctcaaggc cacggagccc gggctgttcg gtgtgtgggc gcacaatggg |
| 2041 | gaggtgcgca ccgccaggct gctgagcgag cgcgacgcag ccaagcacag gctcgtggtg |
| 2101 | cttgtcaagg acaatggcga gcctcctcgc tcggccaccg ccacgctgca cttgctcctg |
| 2161 | gtggacggct tctcccagcc ctacctgcct ctcccggagg cggccccggc ccaggcccag |
| 2221 | gccgaggccg acttgctcac cgtctacctg gtggtggcgt tggcctcggt gtcttcgctc |
| 2281 | ttcctcctct cggtgctcct gttcgtggcg gtgcggctgt gcaggaggag cagggcggcc |
| 2341 | tcggtgggtc gctgctcggt gcccgagggt ccttttccag ggcatctggt ggacgtgagg |
| 2401 | ggcgctgaga ccctgtccca gagctaccag tatgaggtgt gtctgacggg aggccccggg |
| 2461 | accagtgagt tcaagttctt gaaaccagtt atttcggata ttcaggcaca gggccctggg |
| 2521 | aggaagggtg aagaaaattc caccttccga aatagctttg gatttaatat tcagtaaagt |
| 2581 | ctgtttttag tttcatatac ttttggtgtg ttacatagcc atgtttctat tagtttactt |
| 2641 | ttaaatctca aatttaagtt attatgcaac ttcaagcatt attttcaagt agtatacccc |
| 2701 | tgtggtttta caatgtttca tcattttttt gcattaataa caactgggtt taatttaatg |
| 2761 | agtatttttt tctaaatgat agtgttaagg ttttaattct ttccaactgc ccaaggaatt |
| 2821 | aattactatt atatctcatt acagaaatct gaggttttga ttcatttcag agcttgcatc |
| 2881 | tcatgattct aatcacttct gtctatagtg tacttgctct atttaagaag gcatatctac |
| 2941 | atttccaaac tcattctaac attctatata ttcgtgtttg aaaaccatgt catttatttc |
| 3001 | tacatcatgt atttaaaaag aaatatttct ctactactat gctcatgaca aaatgaaaca |
| 3061 | aagcatattg tgagcaatac tgaacatcaa taataccctt agtttatata cttattattt |
| 3121 | tatctttaag catgctactt ttacttggcc aatattttct tatgttaact tttgctgatg |
| 3181 | tataaaacag actatgcctt ataattgaaa taaaattata atctgcctga aaatgaataa |
| 3241 | aaataaaaca ttttgaaatg tgaaaaaaaa aaaaaaaaaa aaaa |

An exemplary human PCDHGA3 amino acid sequence is set forth below (SEQ ID NO: 73; GenBank Accession No: Q9Y5H0.2, Version 2, incorporated herein by reference):

| | |
|---|---|
| 1 | mtnclsfrng rglallcall gtlcetgsgq irysvseeld kgsfvgnian dlgleprela |
| 61 | ergvrivsrg rtqlfslnpq sgslvtaeri dreelcaqip lclvkinilv edklkifeve |
| 121 | ieikdindna pnfpteelei kigeltvpgt rfpiktafdp dvginslqny klspndyfsl |
| 181 | avnsysegak ypelvleral drekkeihql vlvasdggdp vhsgnlhiqv ivldandnpp |
| 241 | mftqpeyrvs vwenvpvgtr lltvnatdpd egfnaqvsyi ldkmpgkiae ifhlnsvsge |
| 301 | vsilksldye damfyeikie aqdgpgllsr akilvtvldv ndnapeitit sltssvpeeg |
| 361 | tvgreialid vhdrdsgqng gvevfvlgnl pfkleksidq yyrlvtatsl dreqiseyni |
| 421 | slrasdggsp plstethitl hvidindnpp tfphlsysay ipennprgas ifsvtaqdpd |
| 481 | snnnaritya ltedtlqgap lssfvsinsn tgvlyalrsf dyeqfrdlkl lvtasdsgnp |
| 541 | plssnvslnl fvldqndnap eilypalptd gstgvelapr saepgylvtk vvavdrdsgq |
| 601 | nawlsyrllk asepglfsvg lhtgevrtar alldrdalkq slvvavqdhg qpplsatvtl |
| 661 | tvavadripd iladlgslep sakpndsdlt lylvvavaav scvflafviv llalrlrrwh |
| 721 | ksrllqasgg glastpgshf vgadgvrafl qtyshevslt adsrkshlif pqpnyadtli |
| 781 | sqesceksep llitqdllem kgdsnllqqa ppntdwrfsq aqrpgtsgsq ngddtgtwpn |
| 841 | nqfdtemlqa milasaseaa dgsstlggga gtmglsaryg pqftlqhvpd yrqnvyipgs |
| 901 | natltnaagk rdgkapaggn gnkkksgkke kk |

An exemplary human PCDHGA3 nucleic acid sequence is set forth below (SEQ ID NO: 74; GenBank Accession No: NM_018916.3, Version 3, incorporated herein by reference):

| | |
|---|---|
| 1 | atgaccaatt gcctgagttt ccgaaatggc agaggactgg ccctgctgtg cgcgctcctg |
| 61 | gggacgctgt gcgaaacagg atccggtcag atccgctact cggtgtctga ggagctagat |
| 121 | aaaggttcct tcgtgggcaa catcgctaac gacctggggc tagagccccg ggagctggcg |
| 181 | gagcgcggag tccgcatcgt ctccagaggt aggacgcagc ttttctctct gaatccgcaa |
| 241 | agcggcagct tggtcaccgc ggagaggata gaccgggagg agctctgcgc tcagatcccg |
| 301 | ctgtgtctgg taaaaattaa cattctggtt gaggataaat tgaaaatttt tgaagtagaa |
| 361 | atagaaatta aagatattaa tgataatgct cctaatttcc caacagagga attggaaata |
| 421 | aaaattggtg aactaacggt tcctggaacc cgatttccaa ttaaaactgc ttttgaccca |
| 481 | gatgtaggca ttaactccct gcagaactac aagcttagcc ccaatgacta cttctctctg |
| 541 | gctgtgaata gcgtctctga gggggccaag tatccagagc tggtgctgga gcgggccctg |
| 601 | gaccgtgaga aaaaagaaat tcaccagctt gtcctggttg cctctgatgg tggcgaccct |
| 661 | gtccactctg gcaacttgca catccaagtg atagtcctgg atgcaaatga caacccacca |
| 721 | atgtttactc agcctgagta ccgtgtgagt gtttgggaga acgtgcctgt gggtacccgg |
| 781 | ctgctcacgg tgaatgccac tgaccctgac gagggattca atgctcaagt gtcttatatt |
| 841 | ctagataaaa tgcctgggaa aatcgctgag attttccatc ttaactcagt gagtggagaa |
| 901 | gtatcaatat taaaaagtct agattatgag gatgccatgt tctatgaaat taaaattgaa |
| 961 | gcacaggatg gaccaggtct tctttcaaga gccaagattc tagtcacggt tctggatgtg |
| 1021 | aatgacaatg ctccagaaat tacaatcacg tctctcacaa gctcagtccc agaagagggc |
| 1081 | accgttggaa gagaaattgc tcttatcgac gtgcatgacc gagattctgg gcagaatggg |
| 1141 | caggttgaag tttttgtcct gggaaatctg ccatttaagt tagaaaaatc aatagatcaa |
| 1201 | tattaccgct tagtgacggc cacatccctg gaccgcgaac aaatatcaga atataacatt |
| 1261 | agtctgagag cctcagatgg gggaagcccg ccactgtcca cagaaactca catcaccctg |
| 1321 | catgtgattg acatcaatga caacccaccc accttccctc atttatccta ctccgcctac |
| 1381 | attccagaaa acaaccccag aggagcctcc atcttctcag tgacagccca ggacccagat |
| 1441 | agcaacaaca acgcccgcat cacttatgca ttgaccgagg acactctcca gggggcgccc |
| 1501 | ctgtcctcct tcgtctctat caactccaac actggcgtcc tatacgcgct gagatccttc |
| 1561 | gactacgagc aatttagaga cttaaagcta ctggtgacag ccagcgacag cgggaaccct |
| 1621 | ccactcagca gcaacgtgtc gctgaacctg ttcgtgctgg accagaacga caacgcgccc |
| 1681 | gagatcctgt accccgccct ccccacagac ggttccactg gcgtggagct ggcgcctcgc |
| 1741 | tccgcagagc ccggctacct ggtgaccaag gtggtggcgg tggacagaga ctcgggccag |
| 1801 | aacgcctggc tgtcctaccg cctgctcaag gccagcgagc cgggactctt ctcggtgggt |
| 1861 | ctgcacacgg gcgaggtgcg cacggcgcga gccctgctgg acagagacgc gctcaagcag |
| 1921 | agcctcgtgg tggccgtcca ggaccacggc cagccccctc tctccgccac tgtcacgctc |
| 1981 | accgtggccg tggccgacag gatccccgac atcctggccg acctgggcag cctcgagccc |
| 2041 | tccgccaaac ccaacgattc ggacctcact ctgtacctgg tggtggcggt ggccgcggtc |
| 2101 | tcctgcgtct tcctggcctt cgtcatcgtg ctgctggcgc tcaggctgcg gcgctggcac |
| 2161 | aagtcacgcc tgctgcaggc ttcgggaggc ggcttggcga gtacgcccgg ctcgcacttt |
| 2221 | gtgggcgcgg acggggttcg ggctttcctg cagacctatt cccacgaggt ctccctcact |
| 2281 | gcggactcgc ggaagagcca cctgattttc ccccagccca actatgcgga cacgctcatc |
| 2341 | agccaggaga gctgtgagaa aagcgagcct cttctgataa ctcaggattt acttgaaatg |
| 2401 | aaaggagatt ccaacctact tcagcaagcc ccgcccaaca cggactggcg tttctctcag |
| 2461 | gcccagagac ccggcaccag cggctcccaa aatggcgatg acaccggcac ctggcccaac |
| 2521 | aaccagtttg acacagagat gctgcaagcc atgatcttgg cgtccgccag tgaagctgct |
| 2581 | gatgggagct ccaccctggg agggggtgcc ggcaccatgg gattgagcgc ccgctacgga |
| 2641 | ccccagttca ccctgcagca cgtgcccgac taccgccaga atgtctacat cccaggcagc |
| 2701 | aatgccacac tgaccaacgc agctggcaag cgggatggca aggccccagc aggtggcaat |
| 2761 | ggcaacaaga agaagtcggg caagaaggag aagaagtaac atggaggcca ggccaagagc |
| 2821 | cacagggcgg cctctcccca accagcccag cttctcctta cctgcaccca ggcctcagag |
| 2881 | tttcagggct aacccccaga atactggtag gggccaaggc catgctcccc ttgggaaaca |
| 2941 | gaaacaagtg cccagtcagc acctacccct tcccccccag ggggttgaat atgcaaaagc |
| 3001 | agttccgctg ggaaccccca tccaatcaac tgctgtaccc atgggggtag tggggttact |
| 3061 | gtagacacca agaaccattt gccacacccc gtttagttac agctgaactc ctccatcttc |
| 3121 | caaatcaatc aggcccatcc atcccatgcc tccctcctcc ccaccccact ccaacagttc |
| 3181 | ctctttcccg agtaaggtgg ttggggtgtt gaagtaccaa gtaacctaca agcctcctag |
| 3241 | ttctgaaaag ttggaagggc atcatgacct cttggcctct cctttgattc tcaatcttcc |
| 3301 | cccaaagcat ggtttggtgc cagccccttc acctccttcc agagcccaag atcaatgctc |
| 3361 | aagttttgga ggacatgatc accatcccca tggtactgat gcttgctgga tttagggagg |
| 3421 | gcattttgct accaagcctc ttcccaacgc cctggggacc agtcttctgt tttgtttttc |
| 3481 | attgtttgac gtttccactg catgccttga cttcccccac ctcctcctca aacaagagac |
| 3541 | tccactgcat gttccaagac agtatggggt ggtaagataa ggaagggaag tgtgtggatg |
| 3601 | tggatggtgg gggcatggac aaagcttgac acatcaagtt atcaaggcct tggaggaggc |
| 3661 | tctgtatgtc ctcaggggac tgacaacatc ctccagattc cagccataaa ccaataacta |
| 3721 | ggctggaccc ttcccactac ataatagggc tcagcccagg cagccagctt tgggctgagc |
| 3781 | taacaggacc aatggattaa actggcattt cagtccaagg aagctcgaag caggtttagg |
| 3841 | accaggtccc cttgagaggt cagaggggcc tctgtgggtg ctgggtactc cagaggtgcc |
| 3901 | actggtggaa gggtcagcgg agccccagca ggaagggtgg gccagccagg ccattcttag |
| 3961 | tccctgggtt ggggaggcag ggagctaggg cagggaccaa atgaacagaa agtctcagcc |
| 4021 | caggatgggg cttcttcaac agggcccctg ccctcctgaa gcctcagtcc ttcaccttgc |
| 4081 | caggtgccgt ttctcttccg tgaaggccac tgcccaggtc cccagtgcgc cccctagtgg |
| 4141 | ccatagcctg gttaaagttc cccagtgcct ccttgtgcat agaccttctt ctcccacccc |
| 4201 | cttctgcccc tgggtccccg gccatccagc ggggctgcca gagaacccca gacctgccct |
| 4261 | tacagtagtg tagcgccccc tccctctttc ggctggtgta gaatagccag tagtgtagtg |
| 4321 | cggtgtgctt ttacgtgatg gcgggtgggc agcgggcggc gggctccgcg cagccgtctg |
| 4381 | tccttgatct gcccgcggcg gcccgtgttg tgttttgtgc tgtgtccacg cgctaaggcg |
| 4441 | accccctccc ccgtactgac ttctcctata agcgcttctc ttcgcatagt cacgtagctc |
| 4501 | ccaccccacc ctcttcctgt gtctcacgca agttttatac tctaatattt atatggcttt |
| 4561 | ttttcttcga caaaaaaata ataaaacgtt tcttctgaaa agctg |

An exemplary human PCDHGB1 amino acid sequence is set forth below (SEQ ID NO: 75; GenBank Accession No: AAI03929.1, Version 1, incorporated herein by reference):

| | |
|---|---|
| 1 | mqrareaemm ksqvlfpfll slfcgaisqq irytipeela ngsrvgklak dlglsvrelp |
| 61 | trklrvsaed yfnvslesgd llvngridre kicgrkleca lefetvaenp mnvfhvvvvi |
| 121 | qdindnaprf vakgidleic esalpgvkfs ldsaqdadve gnslklytin pnqyfslstk |
| 181 | espdgskypv lllekpldre hqsshrlilt amdggdppls gtthiwirvt dandnapvfs |
| 241 | qevyrvslqe nvpwgtsvlr vmatdqdegi naeityafln spistslfnl npntgdittn |
| 301 | gtldfeetsr yvlsveakdg gvhtahcnvq ieivdendna pevtfmsfsn qipedsdlgt |
| 361 | vialikvrdk dsgqngmvtc ytqeevpfkl estsknyykl viagalnreq tadynvtiia |
| 421 | tdkgkpalss rtsitlhisd indnapvfhq asyvvhvsen nppgasiaqv sasdpdlgpn |
| 481 | drvsysilas dleprellsy vsvspqsgvv faqrafdheq lrafeltlqa rdqgspalsa |
| 541 | nvslrvlvgd lndnaprvly palgpdgsal fdmvpraaep gylvtkvvav dadsghnawl |
| 601 | syhvlqasep glfslglrtg evrtaralgd rdaarqrllv avrdggqppl satatlhlif |
| 661 | adslqevlpd lsdrpepsdp qtelqfylvv alalisvlfl lavilaialr lrrsssldte |
| 721 | gcfqtglcsk sgpgvppnhs egtlpysynl ciashsakte fnslnltpem appqdllcdd |
| 781 | psmvvcasne dhkiaydpsl pshvsfckss |

An exemplary human PCDHGB1 nucleic acid sequence is set forth below (SEQ ID NO: 76; GenBank Accession No: NM_018922.2, Version 2, incorporated herein by reference):

| | |
|---|---|
| 1 | atgcagagag ccagagaagc cgaaatgatg aaaagtcagg tactgtttcc cttcctgctg |
| 61 | tctttgttct gcggggccat ctcccagcag atccgataca cgattccaga ggagctagcc |
| 121 | aacggctcac gggtggggaa acttgccaag gatctggggc tcagtgtccg ggagttgcca |
| 181 | actcgaaaac tgcgggttag tgcagaggat tatttcaacg ttagtttgga gagcggggat |
| 241 | ttgttagtga acggtaggat agatcgagag aagatttgcg gaaggaaact tgagtgtgca |
| 301 | ctagaattcg aaacggtcgc tgaaaaccca atgaatgttt tccacgtggt tgttgtaatc |
| 361 | caagatatta atgacaatgc accacgtttc gttgcaaaag gcattgactt agaaatttgt |
| 421 | gagtcagcct tacccggggt aaaattctct ctggattctg ctcaagatgc agatgtggaa |
| 481 | ggcaattcac tgaagttata caccatcaac cccaatcaat acttctctct gtcaacgaag |
| 541 | gaaagtcctg atggaagtaa atatccggta ttactgctgg aaaaacctct agacagggaa |
| 601 | catcagagct ctcatcgctt aatcctgact gccatggatg gcggggaccc gcctctaagc |
| 661 | ggcaccaccc atatctggat ccgagttacg gatgccaatg ataatgctcc cgtgtttagc |
| 721 | caggaggtat acagggttag cctccaagaa aacgtaccgt ggggaacctc cgtgctgcgg |
| 781 | gtgatggcca cagaccagga tgagggcatt aatgcagaga tcacctatgc cttcctcaat |
| 841 | tccccaataa gtaccagcct cttcaatctc aatccaaata ctggcgacat cacaaccaat |
| 901 | ggcacattgg attttgaaga gacaagtaga tatgtgttga gtgtggaagc taaggatgga |
| 961 | ggagtacaca cagctcactg taatgttcaa atagaaattg ttgacgagaa tgacaatgcc |
| 1021 | ccagaggtga cattcatgtc cttctctaac cagattccag aggattcaga ccttggaact |
| 1081 | gtaatagccc tcataaaagt gcgagacaag gattctgggc aaaatggcat ggtgacatgc |
| 1141 | tatactcagg aagaagttcc tttcaaatta gaatccacct cgaagaatta ttacaagctg |
| 1201 | gtgattgctg gagccctaaa ccgggagcag acagcagact acaacgtcac aatcatagcc |
| 1261 | accgacaagg gcaaaccagc cctttcctcc aggacaagca tcaccctgca catctccgac |
| 1321 | atcaacgaca atgcacctgt tttccatcag gcctcctatg tggtccacgt gtctgagaac |
| 1381 | aacccacctg gcgcctccat tgcacaagta agcgcctccg acccggattt gggacccaac |
| 1441 | ggcagagtct cctactctat tctggccagt gacctggagc cgcgggagct gttgtcctac |
| 1501 | gtgtccgtga gcccgcagag cggggtggtg ttcgcgcagc gcgccttcga ccacgagcag |
| 1561 | ctgcgcgcct tcgagctcac actgcaggcc agggaccagg gctcccccgc gctcagcgcc |
| 1621 | aacgtgagcc tgcgcgtgtt ggtgggcgac ctcaatgaca atgcgccacg ggtgctgtac |
| 1681 | cccgcgctgg ggcctgatgg ctccgccctc ttcgatatgg tgccacgcgc cgcagagccc |
| 1741 | ggctacctgg tgaccaaggt ggtggcggtg gacgcagact caggacacaa cgcttggctg |
| 1801 | tcctaccacg tgctgcaggc cagcgagccc gggctcttca gcctggggtt gcgcacgggt |
| 1861 | gaggtgcgca cagcgcgtgc cttgggcgac agggacgcgg cccgccagcg cctgctggtc |
| 1921 | gctgtgcgtg atggaggaca gccgccactc tccgccaccg ccacgctgca cctaatcttc |
| 1981 | gcggatagcc tgcaagaggt attgccagac ctcagcgacc gccctgagcc ctctgacccc |
| 2041 | cagacggaac tgcagtttta cctggttgtg gccttggcct tgatctcagt gctctttctc |
| 2101 | ctcgcggtga ttctagcgat cgccctgcgc ctgcgacgtt cctccagcct cgacactgag |
| 2161 | ggctgctttc aaaccggtct ctgctccaag tctgggcccg gggttcctcc caaccacagc |
| 2221 | gaggggactt tgccctattc ctacaatcta tgtattgcct ctcattctgc aaagacagag |
| 2281 | tttaattctc tcaacctgac accggaaatg gctccccctc aggatctgct gtgtgatgat |
| 2341 | ccttctatgg ttgtatgtgc cagtaatgaa gatcacaaaa tcgcttatga cccttctttg |
| 2401 | tcttcgcacc aagccccgcc caacacggac tggcgtttct ctcaggccca gagacccggc |
| 2461 | accagcggct cccaaaatgg cgatgacacc ggcacctggc ccaacaacca gtttgacaca |
| 2521 | gagatgctgc aagccatgat cttggcgtcc gccagtgaag ctgctgatgg gagctccacc |
| 2581 | ctgggagggg gtgccggcac catgggattg agcgcccgct acggacccca gttcaccctg |
| 2641 | cagcacgtgc ccgactaccg ccagaatgtc tacatcccag gcagcaatgc cacactgacc |
| 2701 | aacgcagctg gcaagcggga tggcaaggcc ccagcaggtg gcaatggcaa caagaagaag |
| 2761 | tcgggcaaga aggagaagaa gtaacatgga ggccaggcca agagccacag ggcggcctct |
| 2821 | ccccaaccag cccagcttct ccttacctgc acccaggcct cagagtttca gggctaaccc |
| 2881 | ccagaatact ggtaggggcc aaggccatgc tccccttggg aaacagaaac aagtgcccag |
| 2941 | tcagcaccta ccccttcccc cccagggggt tgaatatgca aaagcagttc cgctgggaac |
| 3001 | ccccatccaa tcaactgctg tacccatggg ggtagtgggg ttactgtaga caccaagaac |
| 3061 | catttgccac accccgttta gttacagctg aactcctcca tcttccaaat caatcaggcc |
| 3121 | catccatccc atgcctccct cctccccacc ccactccaac agttcctctt tcccgagtaa |
| 3181 | ggtggttggg gtgttgaagt accaagtaac ctacaagcct cctagttctg aaaagttgga |
| 3241 | agggcatcat gacctcttgg cctctccttt gattctcaat cttcccccaa agcatggttt |
| 3301 | ggtgccagcc ccttcacctc cttccagagc ccaagatcaa tgctcaagtt ttggaggaca |
| 3361 | tgatcaccat ccccatggta ctgatgcttg ctggatttag ggagggcatt ttgctaccaa |
| 3421 | gcctcttccc aacgccctgg ggaccagtct tctgttttgt ttttcattgt ttgacgtttc |
| 3481 | cactgcatgc cttgacttcc cccacctcct cctcaaacaa gagactccac tgcatgttcc |
| 3541 | aagacagtat ggggtggtaa gataaggaag ggaagtgtgt ggatgtggat ggtgggggca |
| 3601 | tggacaaagc ttgacacatc aagttatcaa ggccttggag gaggctctgt atgtcctcag |
| 3661 | gggactgaca acatcctcca gattccagcc ataaaccaat aactaggctg gacccttccc |
| 3721 | actacataat agggctcagc ccaggcagcc agctttgggc tgagctaaca ggaccaatgg |
| 3781 | attaaactgg catttcagtc caaggaagct cgaagcaggt ttaggaccag gtccccttga |
| 3841 | gaggtcagag gggcctctgt gggtgctggg tactccagag gtgccactgg tggaagggtc |
| 3901 | agcggagccc cagcaggaag ggtgggccag ccaggccatt cttagtccct gggttgggga |
| 3961 | ggcagggagc tagggcaggg accaaatgaa cagaaagtct cagcccagga tggggcttct |
| 4021 | tcaacagggc ccctgccctc ctgaagcctc agtccttcac cttgccaggt gccgtttctc |
| 4081 | ttccgtgaag gccactgccc aggtccccag tgcgccccct agtggccata gcctggttaa |
| 4141 | agttccccag tgcctccttg tgcatagacc ttcttctccc acccccttct gcccctgggt |
| 4201 | ccccggccat ccagcggggc tgccagagaa ccccagacct gcccttacag tagtgtagcg |
| 4261 | ccccctccct ctttcggctg gtgtagaata gccagtagtg tagtgcggtg tgcttttacg |
| 4321 | tgatggcggg tgggcagcgg gcggcgggct ccgcgcagcc gtctgtcctt gatctgcccg |
| 4381 | cggcggcccg tgttgtgttt tgtgctgtgt ccacgcgcta aggcgacccc ctcccccgta |
| 4441 | ctgacttctc ctataagcgc ttctcttcgc atagtcacgt agctcccacc ccaccctctt |
| 4501 | cctgtgtctc acgcaagttt tatactctaa tatttatatg gctttttttc ttcgacaaaa |
| 4561 | aaataataaa acgtttcttc tgaaaagctg |

An exemplary human PCDHGB2 amino acid sequence is set forth below (SEQ ID NO: 77; GenBank Accession No: AAI01806.1, Version 1, incorporated herein by reference):

| | |
|---|---|
| 1 | mkassgrcgl vrwlqvllpf llslfpgalp vqirysipee laknsvvgnl akdlglsvrd |
| 61 | lparklrvsa ekeyftvnpe sgdllvsdri dreqicgkqp lcvldfdtva enplnifyia |
| 121 | vivqdindnt plfkqtkinl kigestkpgt tfpldpalds dvgpnslqry hlndneyfdl |
| 181 | aekqtpdgrk ypelilkhsl dreehslhql vltavdggdp pgsgttqiri kvtdandnpp |
| 241 | vfsqdvyrvt lredvppgff vlqvtatdrd eginaeitys fhnvdeqvkh ffnlnektge |
| 301 | ittkddldfe iassytlsie akdpgdlaah csiqveildd ndcapevivt svstplpeds |
| 361 | ppgtvialik trdrdsgeng evycqvlgna kfilkssskn yyklvtdgal dreeipeynl |
| 421 | titatdggkp plsssiivtl hisdvndnap vfqqtsymvh vaennppgas iaqisasdpd |
| 481 | lgpsgqvsys ivasdlkpre ilsyvsvsaq sgvvfaqraf dheqlrafel tlqardqgsp |
| 541 | alsanvslrv lvgdlndnap rvlypalgpd gsalfdmvpr aaepgylvtk vvavdadsgh |
| 601 | nawlsyhvlq asepglfslg lrtgevrtar algdrdaarq rllvavrdgg qpplsatatl |
| 661 | hlifadslqe vlpdlsdrre psdpqaklqf ylvvalalis vlfflavila islrlrlssr |
| 721 | sdawdcfqpg lsskpgpgvl pnysegtlpy synlcvasqs aktefnflni tpelvpaqdl |
| 781 | vcdnasweqn tnhgaagvpf asdtilkvsf n |

An exemplary human PCDHGB2 nucleic acid sequence is set forth below (SEQ ID NO: 78; GenBank Accession No: NM_018923.2, Version 2, incorporated herein by reference):

| | |
|---|---|
| 1 | atgaaagcga gctcagggag gtgcgggctg gtgcggtggc tgcaggtact gttgcccttc |
| 61 | ctgttgtctt tgttccccgg ggctctccca gtccagatcc gctattcaat tccagaggag |
| 121 | ctggccaaaa actcggtcgt aggaaacctc gccaaggatc tggggctcag cgtccgggac |
| 181 | ttgccagccc ggaagctgcg ggttagcgcg gagaaggaat atttcacagt aaacccagaa |
| 241 | agcggagact tacttgtgag tgacagaata gaccgagaac agatatgcgg gaagcagcct |
| 301 | ctgtgtgttc tggatttcga tactgtcgct gaaaatccac taaatatttt ctacatagca |
| 361 | gtaattgtgc aggatataaa tgataatacc ccgctattca aacagactaa gattaattta |
| 421 | aaaattggcg aatccactaa gccaggtaca acatttccac ttgacccagc cctggattca |
| 481 | gatgttggtc ctaactcact acaaagatac caccttaatg acaacgagta ctttgatctc |
| 541 | gctgagaaac agactccaga tggtcgtaaa tatcctgagt tgattctaaa acactctctg |
| 601 | gacagagaag agcacagttt acatcaattg gtcctcacag ctgtggatgg cggagaccca |
| 661 | cctcaaagtg gcacgaccca aatccgaatc aaagtcacgg atgccaacga taaccctcca |
| 721 | gtgttcagcc aggacgtgta cagggtcacc ctgagggagg acgtgccgcc gggcttcttt |
| 781 | gtgcttcaag tgacagccac cgaccgggat gaaggcataa acgcagagat cacctactcc |
| 841 | tttcataatg tggacgaaca agtgaaacac tttttcaact taaatgaaaa aacaggagaa |
| 901 | atcacgacaa aggatgattt ggattttgag attgcaagta gttacactct gagtatcgaa |
| 961 | gcaaaagatc ctggagatct agcagcccac tgcagtatcc aagttgaaat tcttgatgac |
| 1021 | aacgattgtg cacctgaagt tattgtgact tcagtatcta ctcccctacc ggaggattcg |
| 1081 | ccaccaggaa cagtgatcgc cttgataaaa acgagagaca gagactctgg agaaaatgga |
| 1141 | gaagtttact gccaagtgtt gggaaatgcc aagtttattt tgaaatcttc ctcaaagaac |
| 1201 | tattacaaac tagtgacaga cggcgctctg gaccgggagg agatcccaga atacaatctc |
| 1261 | accatcacag ccaccgacgg gggcaagccg cccctctcct ccagcataat tgtcaccctg |
| 1321 | cacatctccg acgtcaacga taatgcccca gttttccaac agacttccta catggttcac |
| 1381 | gtggcagaga acaatcctcc tggcgcctct atcgctcaaa tcagtgcctc tgaccctgac |
| 1441 | ttgggcccca gtggccaagt ttcctactcc atcgtagcga gcgacctgaa gccgcgggag |
| 1501 | attttatcct acgtgtccgt gagcgcgcag agcggggtgg tgttcgcgca gcgcgccttc |
| 1561 | gatcatgagc agctgcgcgc cttcgagctc acactgcagg cccgcgacca gggctcgccc |
| 1621 | gcgctcagcg ccaacgtgag cctgcgcgtg ttagtgggcg acctcaatga caatgcgcca |
| 1681 | cgggtgctgt accccgcgct ggggcctgat ggctccgccc tcttcgatat ggtgccacgc |
| 1741 | gccgcagagc ccggctacct ggtgaccaag gtggtggcgg tggacgcaga ctcaggacac |
| 1801 | aacgcttggc tgtcctacca cgtgctgcag gccagcgagc ccgggctctt cagcctgggg |
| 1861 | ttgcgcacgg gtgaggtgcg cacagcgcgt gccttgggcg acagggacgc ggcccgccag |
| 1921 | cgcctgctgg tcgctgtgcg tgatggagga cagccgccac tctccgctac ggccacgctg |
| 1981 | cacctaatct tcgcggatag cctgcaagag gtattgccag acctcagcga ccgccgggag |
| 2041 | ccctctgacc cccaggcaaa actgcagttt tacctggttg tggccttggc cttgatctca |
| 2101 | gtgctcttct tcctcgcggt gattctggca atctccctgc gcctgcgact ctcttccagg |
| 2161 | tcagatgctt gggactgttt tcagcctggt ctcagctcca agcctggacc tggggttctc |
| 2221 | cccaattaca gtgagggtac attgccctat tcctacaacc tgtgtgttgc ctcacaatca |
| 2281 | gccaagacag agttcaattt tctgaacata accccggaat tggttcccgc gcaagatctc |
| 2341 | gtctgtgaca atgcctcttg ggaacaaaat acaaatcatg gagccgctgg ggtccctttt |
| 2401 | gcctcagata ctattttgaa gcaagccccg cccaacacgg actggcgttt ctctcaggcc |
| 2461 | cagagacccg gcaccagcgg ctcccaaaat ggcgatgaca ccggcacctg gcccaacaac |
| 2521 | cagtttgaca cagagatgct gcaagccatg atcttggcgt ccgccagtga agctgctgat |
| 2581 | gggagctcca ccctgggagg gggtgccggc accatgggat tgagcgcccg ctacggaccc |
| 2641 | cagttcaccc tgcagcacgt gcccgactac cgccagaatg tctacatccc aggcagcaat |
| 2701 | gccacactga ccaacgcagc tggcaagcgg gatggcaagg ccccagcagg tggcaatggc |
| 2761 | aacaagaaga agtcgggcaa gaaggagaag aagtaacatg gaggccaggc caagagccac |
| 2821 | agggcggcct ctccccaacc agcccagctt ctccttacct gcacccaggc ctcagagttt |
| 2881 | cagggctaac ccccagaata ctggtagggg ccaaggccat gctccccttg ggaaacagaa |
| 2941 | acaagtgccc agtcagcacc taccccttcc cccccagggg gttgaatatg caaaagcagt |
| 3001 | tccgctggga acccccatcc aatcaactgc tgtacccatg ggggtagtgg ggttactgta |
| 3061 | gacaccaaga accatttgcc acaccccgtt tagttacagc tgaactcctc catcttccaa |
| 3121 | atcaatcagg cccatccatc ccatgcctcc ctcctcccca ccccactcca acagttcctc |
| 3181 | tttcccgagt aaggtggttg gggtgttgaa gtaccaagta acctacaagc ctcctagttc |
| 3241 | tgaaaagttg gaagggcatc atgacctctt ggcctctcct ttgattctca atcttccccc |
| 3301 | aaagcatggt ttggtgccag ccccttcacc tccttccaga gcccaagatc aatgctcaag |
| 3361 | ttttggagga catgatcacc atccccatgg tactgatgct tgctggattt agggagggca |
| 3421 | ttttgctacc aagcctcttc ccaacgccct ggggaccagt cttctgtttt gtttttcatt |
| 3481 | gtttgacgtt tccactgcat gccttgactt cccccacctc ctcctcaaac aagagactcc |
| 3541 | actgcatgtt ccaagacagt atggggtggt aagataagga agggaagtgt gtggatgtgg |
| 3601 | atggtggggg catggacaaa gcttgacaca tcaagttatc aaggccttgg aggaggctct |
| 3661 | gtatgtcctc aggggactga caacatcctc cagattccag ccataaacca ataactaggc |
| 3721 | tggacccttc ccactacata atagggctca gcccaggcag ccagctttgg gctgagctaa |
| 3781 | caggaccaat ggattaaact ggcatttcag tccaaggaag ctcgaagcag gtttaggacc |
| 3841 | aggtcccctt gagaggtcag aggggcctct gtgggtgctg ggtactccag aggtgccact |
| 3901 | ggtggaaggg tcagcggagc cccagcagga agggtgggcc agccaggcca ttcttagtcc |
| 3961 | ctgggttggg gaggcaggga gctagggcag ggaccaaatg aacagaaagt ctcagcccag |
| 4021 | gatggggctt cttcaacagg gcccctgccc tcctgaagcc tcagtccttc accttgccag |
| 4081 | gtgccgtttc tcttccgtga aggccactgc ccaggtcccc agtgcgcccc ctagtggcca |
| 4141 | tagcctggtt aaagttcccc agtgcctcct tgtgcataga ccttcttctc ccaccccctt |
| 4201 | ctgcccctgg gtccccggcc atccagcggg gctgccagag aaccccagac ctgcccttac |
| 4261 | agtagtgtag cgccccctcc ctctttcggc tggtgtagaa tagccagtag tgtagtgcgg |
| 4321 | tgtgctttta cgtgatggcg ggtgggcagc gggcggcggg ctccgcgcag ccgtctgtcc |
| 4381 | ttgatctgcc cgcggcggcc cgtgttgtgt tttgtgctgt gtccacgcgc taaggcgacc |
| 4441 | ccctcccccg tactgacttc tcctataagc gcttctcttc gcatagtcac gtagctccca |
| 4501 | ccccaccctc ttcctgtgtc tcacgcaagt tttatactct aatatttata tggctttttt |
| 4561 | tcttcgacaa aaaaataata aaacgtttct tctgaaaagc tg |

An exemplary human EMILIN1 amino acid sequence is set forth below (SEQ ID NO: 79; GenBank Accession No: AAH07530.1, Version 1, incorporated herein by reference):

| | |
|---|---|
| 1 | madlgatkdr iiseinrlqq eatehatese erfrgleegq aqagqcpsle grlgrlegvc |
| 61 | erldtvaggl qglreglsrh vaglwaglre tnttsqmqaa lleklvggqa glgrrlgaln |
| 121 | sslqlledrl hqlslkdltg egtkgpagea gppgppglqg ppgpagppgs pgkdgqegpi |
| 181 | gppgpqgeqg vegapaapvp qvafsaalsl prsepgtvpf drvllndggy ydpetgvfta |
| 241 | plagryllsa vltghrhekv eavlsrsnqg varvdsggye peglenkpva esqpspgtlg |
| 301 | vfslilplqa gdtvcvdlvm gqlahseepl tifsgallyg dpeleha |

An exemplary human EMILIN1 nucleic acid sequence is set forth below (SEQ ID NO: 80; GenBank Accession No: NM_007046.3, Version 3, incorporated herein by reference):

| | |
|---|---|
| 1 | gggaggggag ccagcaggga ggaggaggcc agggcccgcc ccacagccac tctcgcgcct |
| 61 | ccgaacagcc acaggggcaa agccctgtca cccccaggat ccggtcatca gggaaagagg |
| 121 | acagggagac cagaagaggg ccagctggga cgagggggcg gacgcccagg aggcaacttc |
| 181 | tgagacgcag ctcctgagag gggcagggac caggcgcggg aggccagagg gggcacagag |
| 241 | aacaaacccc ctcagaagtg aagaggagag cggaaggaac cgagagggga cggacaggag |
| 301 | ctgaggagga aagaggaggg gagaggggtc aggccaggca gccaaggaga agacgtgtgg |
| 361 | ccgggggcta tcagaaggaa actgggacgg acgggccggg ctcgggctgt cctgtggagc |
| 421 | agcagcatcc ccggggccgg cagaggcgcc agtggctggg cgggatgagt ctctgagggc |
| 481 | cactgtggag cgccccgcca tggccccccg caccctctgg agctgctacc tctgctgcct |
| 541 | gctgacggca gctgcagggg ccgccagcta ccctcctcga ggtttcagcc tctacacagg |
| 601 | ttccagtggg gccctcagcc ccggggggcc ccaggcccag attgcccccc ggccagccag |
| 661 | ccgccacagg aactggtgtg cctacgtggt gacccggaca gtgagctgtg tccttgagga |
| 721 | tggagtggag acatatgtca agtaccagcc ttgtgcctgg ggccagcccc agtgtcccca |
| 781 | aagcatcatg taccgccgct tcctccgccc tcgctaccgt gtggcctaca agacagtgac |
| 841 | cgacatggag tggaggtgct gtcagggtta tgggggcgat gactgtgctg agagtcccgc |
| 901 | tccagcgctg gggcctgcgt cttccacacc acggcccctg gcccggcctg cccgccccaa |
| 961 | cctctctggc tccagtgcag gcagccccct cagtggactg gggggagaag gtcctgggga |
| 1021 | gtcagagaag gtgcagcagc tggaggaaca ggtgcagagc ctgaccaagg agctgcaagg |
| 1081 | cctgcggggc gtcctgcaag gactgagcgg gcgcctggca gaggatgtgc agagggctgt |
| 1141 | ggagacggcc ttcaacggga ggcagcagcc agctgacgcg gctgcccgcc ctggggtgca |
| 1201 | tgaaaccctc aatgagatcc agcaccagct gcagctcctg gacacccgcg tctccaccca |
| 1261 | cgaccaggag ctgggtcacc tcaacaacca tcatggcggc agcagcagca gtgggggcag |
| 1321 | cagggcccca gccccagcct cagcccctcc gggccccagt gaggagctgc tgcggcagct |
| 1381 | ggagcagcgg ttgcaggagt cctgctccgt gtgcctggcc gggctagatg gcttccgccg |
| 1441 | gcagcagcag gaggacaggg agcggctgcg agcgatggag aagctgctgg cctcggtgga |
| 1501 | ggagcggcaa cggcacctcg cagggctggc ggtgggccgc aggccccctc aggaatgctg |
| 1561 | ctctccagag ctgggccggc gactggcaga gctggagcgc aggctggatg tcgtggccgg |
| 1621 | ctcagtgaca gtgctgagtg ggcggcgagg cacagagctg ggaggagccg cggggcaggg |
| 1681 | aggccacccc ccaggctaca ccagcttggc ctcccgcctg tctcgcctgg aggaccgctt |
| 1741 | caactccacc ctgggccctt cggaggagca ggaggagagc tggcctgggg ctcctggggg |
| 1801 | gctgagccac tggctgcctg ctgcccgggg ccgactagag cagttggggg ggctgctggc |
| 1861 | caatgtgagc ggggagctgg gggggcggtt ggatctgttg gaggagcagg tggcaggggc |
| 1921 | catgcaggca tgcgggcagc tctgctctgg ggcccctggg gagcaggact ctcaagtcag |
| 1981 | cgagatcctc agtgccttgg agcgcagggt gctggacagt gaggggcagc tgcggctggt |
| 2041 | gggctccggc ctgcacacgg tggaagcagc gggggaggcc cggcaggcca cgctggaggg |
| 2101 | attacaagag gttgtgggcc ggctccagga tcgtgtggat gcccaggatg agacagctgc |
| 2161 | agagttcaca ctacggctga atctcactgc ggcccggcta ggccaactgg aggggctgct |
| 2221 | gcaggcccat ggggatgagg gctgtggggc ctgtggcgga gtccaagagg aactaggccg |
| 2281 | ccttcgggat ggtgtggagc gctgctcctg ccccctgttg cctcctcggg gtcctggggc |
| 2341 | tggtccaggt gttgggggcc caagccgtgg gcccctggac ggcttcagcg tgtttggggg |
| 2401 | cagctcaggc tcagccctgc aggccctgca aggagagctc tctgaggtta ttctcagctt |
| 2461 | cagctccctc aatgactcac tgaatgagct ccagaccact gtggagggcc agggcgctga |
| 2521 | tctggctgac ctgggggcaa ccaaggaccg tatcatttct gagattaaca ggctgcagca |
| 2581 | ggaggccaca gagcatgcta cagagagtga agagcgcttc cgaggcctag aggagggaca |
| 2641 | agcacaggcc ggccagtgcc ccagcttaga ggggcgattg ggccgtcttg agggtgtctg |
| 2701 | tgaacggttg gacactgtgg ctgggggact gcagggcctg cgcgagggcc tttccagaca |
| 2761 | cgtggctggg ctctgggctg ggctccggga aaccaacacc accagccaga tgcaggcagc |
| 2821 | cctgctggag aagctggtcg ggggacaggc gggcctgggc aggcggctgg gtgcccttaa |
| 2881 | cagctccctg cagctcctgg aggaccgtct gcaccagctc agcctgaagg acctcactgg |
| 2941 | gcctgcagga gaggctgggc ccccagggcc tcctgggctg cagggacccc caggccctgc |
| 3001 | tggacctcca ggatcaccag gcaaggacgg gcaagagggc cccatcgggc caccaggtcc |
| 3061 | tcaaggtgaa cagggagtgg agggggcacc agcagcccct gtgccccaag tggcattttc |
| 3121 | agctgctctg agtttgcccc ggtctgaacc aggcacggtc cccttcgaca gagtcctgct |
| 3181 | caatgatgga ggctattatg atccagagac aggcgtgttc acagcgccac tggctggacg |
| 3241 | ctacttgctg agcgcggtgc tgactgggca ccggcacgag aaagtggagg ccgtgctgtc |
| 3301 | ccgctccaac cagggcgtgg cccgcgtaga ctccggtggc tacgagcctg agggcctgga |
| 3361 | gaataagccg gtggccgaga gccagcccag cccgggcacc ctgggcgtct tcagcctcat |
| 3421 | cctgccgctg caggccgggg acacggtctg cgtcgacctg gtcatggggc agctggcgca |
| 3481 | ctcggaggag ccgctcacca tcttcagcgg ggccctgctc tatggggacc cagagcttga |
| 3541 | acacgcgtag actggggtcc cgcccgacgt gtctacgtcg gctgaagaga cagcgggggc |
| 3601 | ggcgggctcc tggggtctcg cctgagacgg ggcacctagc cctgggcgag cgccgcaccc |
| 3661 | gggcccgcag cggcaccgcg cccagagcgg cctctcccca cgcccggggc gcgccggctc |
| 3721 | agggaggctc ggggccgccc atgcagactt ttggcctggc gcgatccccc aagaacccct |
| 3781 | ccagggccgg cctgcggagg agccgatcct cgcaccctcc gctccctcca ctggccctcc |
| 3841 | aggtcgattc cctgggctcc aggctccccc gcgcgggcgc cgcccaccgc catactaaac |
| 3901 | gatcgaggaa taaagacact tggtttttct aaaaaaaaaa aaaaaaaaaa aaaaaaaaa |

An exemplary human TNN amino acid sequence is set forth below (SEQ ID NO: 81; GenBank Accession No: AAI36620.1, Version 1, incorporated herein by reference):

| | |
|---|---|
| 1 | mslqemfrfp mglllgsvll vasapatlep pgcsnkeqqv tvshtykidv pksalvqvda |
| 61 | dpqplsddga sllalgeare eqniifrhni rlqtpqkdce lagsvqdlla rvkkleeemv |
| 121 | emkeqcsaqr ccqgvtdlsr hcsghgtfsl etcschceeg regpacerla cpgacsghgr |
| 181 | cvdgrclche pyvgadcgyp acpencsghg ecvrgvcqch edfmsedcse krcpgdcsgh |
| 241 | gfcdtgecyc eegftgldca qvvtpqglql lkntedsllv swepssqvdh yllsyyplgk |
| 301 | elsgkqiqvp keqhsyeilg llpgtkyivt lrnvknevss spqhllattd lavlgtawvt |
| 361 | detensldve wenpstevdy yklrygpmtg qevaevtvpk ssdpksrydi tglhpgteyk |
| 421 | itvvpmrgel egkpillngr teidsptnvv tdrvtedtat vswdpvqavi dkyvvrytsa |
| 481 | dgdtkemavh kdesstvltg lkpgeaykvy vwaergnqgs kkadtnalte idspanlvtd |
| 541 | rvtentatis wdpvqatidk yvvrytsadd qetrevlvgk eqsstvltgl rpgveytvhv |
| 601 | waqkgdresk kadtnaptdi dspknlvtdr vtenmatvsw dpvqaaidky vvrytsagge |
| 661 | trevpvgkeq sstvltglrp gmeymvhvwa qkgdqeskka dtkaqtdids pqnlvtdrvt |
| 721 | enmatvswdp vratidryvv rytsakdget revpvgkeqs stvltglrpg veytvhvwaq |
| 781 | kgaqeskkad tkaqtdidsp qnlvtdwvte ntatvswdpv qatidryvvh ytsangetre |
| 841 | vpvgkeqsst vltglrpgme ytvhvwaqkg nqeskkadtk aqteidgpkn lvtdwvtenm |
| 901 | atvswdpvqa tidkymvryt sadgetrevp vgkehsstvl tglrpgmeym vhvwaqkgaq |
| 961 | eskkadtkaq teldpprnlr psavtqsggi ltwtppsaqi hgyiltyqfp dgtvkemqlg |
| 1021 | redqrfalqg leqgatypvs lvafkggrrs rnvsttlstv garfphpsdc sqvqqnsnaa |
| 1081 | sglytiylhg dasrplqvyc dmetdgggwi vfqrrntgql dffkrwrsyv egfgdpmkef |
| 1141 | wlgldklhnl ttgtparyev rvdlqtanes ayaiydffqv asskeryklt vgkyrgtagd |
| 1201 | altyhngwkf ttfdrdndia lsncalthhg gwwyknchla npngrygetk hsegvnwepw |
| 1261 | kghefsipyv elkirphgys repvlgrkkr tlrgrlrtf |

An exemplary human TNN nucleic acid sequence is set forth below (SEQ ID NO: 82; GenBank Accession No: NM_022093.1, Version 1, incorporated herein by reference):

| | |
|---|---|
| 1 | aagtaccaag gtctgcggca ggaggagacc ggctcacagg agcagcagca ttggaagagg |
| 61 | cacccagcag cctcccaggc atcctggagg gtctgctccc tgtctttcca aggatgagtc |
| 121 | tccaggagat gttccgcttc cctatggggc tcctgcttgg ctctgtgctc ctggtggctt |
| 181 | cggccccagc cactctggag cctcccggct gcagcaacaa ggagcaacag gtcactgtca |
| 241 | gccacaccta caagatcgat gtgcccaagt ctgccttggt tcaggttgac gctgaccctc |
| 301 | agcccctcag tgacgatggg gcttcgctct tggccctggg ggaggccagg gaggaacaga |
| 361 | acatcatctt caggcacaac atccgccttc agacgccaca gaaggactgc gagttggcag |
| 421 | gcagtgtcca ggacctcctg gcccgggtga agaagctgga ggaagagatg gtggagatga |
| 481 | aggaacagtg tagtgcccag cgctgctgcc agggagtcac tgatctaagc cgccactgca |
| 541 | gcggccacgg gaccttctcc ctggagacct gcagctgcca ctgcgaagag ggcagggagg |
| 601 | gccccgcctg cgagcggctg gcctgccccg gggcgtgcag cggccacggg cgttgcgtgg |
| 661 | acgggcgctg cctgtgccat gagccctacg tgggtgccga ctgcggctac ccggcctgcc |
| 721 | ctgagaactg cagcggacac ggcgagtgcg tgcgcggcgt gtgccagtgc cacgaagact |
| 781 | tcatgtcgga ggactgcagc gagaagcgct gtcccggcga ctgcagcggc cacggcttct |
| 841 | gtgacacggg cgagtgctac tgcgaggagg gcttcacagg cctggactgt gcccaggtgg |
| 901 | tcaccccaca gggcctgcag ctgctcaaga acacggagga ttctctgctg gtgagctggg |
| 961 | agccctccag ccaggtggat cactacctcc tcagctacta ccccctgggg aaggagctct |
| 1021 | ctgggaagca gatccaagtg cccaaggagc agcacagcta tgagattctt ggtttgctgc |
| 1081 | ctggaaccaa gtacatagtc accctgcgta acgtcaagaa tgaagtttct agcagcccac |
| 1141 | agcatctact tgccaccaca gaccttgctg tgcttggcac tgcctgggtg acagatgaga |
| 1201 | ctgagaactc ccttgacgtg gagtgggaaa acccctcaac tgaggtggac tactacaagc |
| 1261 | tgcgatatgg ccccatgaca ggacaggagg tagctgaggt cactgtgccc aagagcagtg |
| 1321 | accccaagag ccgatatgac atcactggtc tgcacccggg gactgagtat aagatcacgg |
| 1381 | tggtgcccat gagaggagag ctggagggca agccgatcct cctgaatggc aggacagaaa |
| 1441 | ttgacagtcc aaccaatgtt gtcactgatc gagtgactga agacacagca actgtctcct |
| 1501 | gggacccagt gcaggctgtc atagacaagt atgtagtgcg ctacacttct gctgatgggg |
| 1561 | acaccaagga aatggcagtg cacaaggatg agagcagcac tgtcctgacg ggcctgaagc |
| 1621 | caggagaggc atacaaggtc tacgtgtggg ctgaaagggg caaccagggg agcaagaaag |
| 1681 | ctgacaccaa tgccctcaca gaaattgaca gcccagcaaa cctggtgact gaccgggtga |
| 1741 | ctgagaatac cgccaccatc tcctgggacc cggtacaggc caccattgac aagtacgtgg |
| 1801 | tgcgctacac ctctgctgac gaccaagaga ccagagaggt tctggtgggg aaggagcaga |
| 1861 | gcagcactgt cctgacaggc ctgaggccag gtgtggagta cacagtgcat gtctgggccc |
| 1921 | agaaggggga ccgagagagc aagaaggctg acaccaacgc cccgacagat attgacagcc |
| 1981 | ccaaaaacct ggtgactgac cgggtgacag agaatatggc cacggtctcc tgggacccgg |
| 2041 | tgcaggccgc cattgacaag tacgtggtgc gctacacctc tgctggtgga gagaccaggg |
| 2101 | aggttccggt ggggaaggag cagagcagca cagtcctgac aggcctgaga ccgggtatgg |
| 2161 | agtacatggt gcacgtgtgg gcccagaagg gggaccagga gagcaagaag gccgacacca |
| 2221 | aggcccagac agacattgac agcccccaaa acctggtgac cgaccgggtg acagagaata |
| 2281 | tggccactgt ctcctgggac ccggtgcggg ccaccattga caggtatgtg gtgcgctaca |
| 2341 | cctctgccaa ggacggagag accagggagg ttccggtggg gaaggagcag agtagcactg |
| 2401 | tcctgacggg cctgaggccg ggtgtggagt acacggtgca cgtgtgggcc cagaaggggg |
| 2461 | cccaggagag caagaaggct gacaccaagg cccagacaga cattgacagc ccccaaaacc |
| 2521 | tggtcactga ctgggtgaca gagaatacag ccactgtctc ctgggacccg gtgcaggcca |
| 2581 | ccattgacag gtatgtggtg cactacacgt ctgccaacgg agagaccagg gaggttccag |
| 2641 | tggggaagga gcagagcagc actgtcctga cgggcctgag gccgggcatg gagtacacgg |
| 2701 | tgcacgtgtg ggcccagaag gggaaccagg agagcaagaa ggctgacacc aaggcccaga |
| 2761 | cagaaattga cggccccaaa aacctagtga ctgactgggt gacggagaat atggccactg |
| 2821 | tctcctggga cccggttcag gccaccattg acaagtacat ggtgcgctac acctctgctg |
| 2881 | acggagagac cagggaggtt ccggtgggga aggagcacag cagcactgtc ctgacgggcc |
| 2941 | tgagaccagg catggagtac atggtgcacg tgtgggccca gaagggggcc caggagagca |
| 3001 | agaaggctga caccaaggcc cagacagaac tcgaccctcc cagaaacctt cgtccatctg |
| 3061 | ctgtaacgca gtctggtggc atattgacct ggacgccccc ctctgctcag atccacggct |
| 3121 | acattctgac ttaccagttc ccagatggca cagttaagga gatgcagctg ggacgggaag |
| 3181 | accagaggtt tgcgttgcaa ggccttgagc aaggcgccac ctaccctgtc tcccttgttg |
| 3241 | cctttaaggg tggtcgccgg agcagaaatg tatccaccac cctctccaca gttggtgccc |
| 3301 | gtttcccaca cccttcggac tgcagtcagg ttcagcagaa cagcaatgcc gccagtggtc |
| 3361 | tgtacaccat ctacctgcat ggcgatgcca gccggcccct gcaggtgtac tgtgacatgg |
| 3421 | aaacggacgg aggtggctgg attgtcttcc agaggcggaa cactgggcag ctggatttct |
| 3481 | tcaagcgatg gaggagctat gtggaaggct ttggggaccc catgaaggag ttctggcttg |
| 3541 | gacttgacaa gctacacaac ctcaccaccg gcactccagc gcggtatgag gtgagagtgg |
| 3601 | atttacagac tgccaatgaa tctgcctatg ctatatatga tttcttccaa gtggcctcca |
| 3661 | gcaaggagcg gtataagctg acagttggga aatacagagg cacggcaggg gatgctctta |
| 3721 | cttaccacaa tggatggaag tttacaactt ttgacagaga caatgatatc gcactcagca |
| 3781 | actgtgccct gacacatcat ggtggctggt ggtataagaa ctgccacttg gccaacccta |
| 3841 | atggcagata tggggagacc aagcacagtg agggggtgaa ctgggagcct tggaaaggac |
| 3901 | atgaattctc cattccttac gtggagttga aaatccgccc tcatggctac agcagggagc |
| 3961 | ctgtcctggg cagaaagaag cggacgctga gaggaaggct gcgaacgttc tgatggcccg |
| 4021 | tgtgagcagt cctcgcagga gacaccacca gctgtggcag cttggggcgg ggtgggtagt |
| 4081 | ggtcactgcg gtctgggagt gctcagatag cccgcagaac aaatcatgtc accaagcttc |
| 4141 | aagccatgga ggttccttcc ctctcacctg catttttgcc cgtctttatg agggtcttga |
| 4201 | aaatcaaaat agtagttgca cagtatgtgt aggaaagaca gtactggaac ggcaaggttt |
| 4261 | ctcagcttat cttcagcaac atatatactg gattagggca agagaaggaa tcacccagca |
| 4321 | cttcaccagt tggaaatctc tggaaattta catctatgta tttaaagttc tgctaatgca |
| 4381 | aatcttttct ctggaaagaa gcacagagga ggagttctga tgacccaggg gttagggctg |
| 4441 | agacaaccgg acgtttgtca cctcctttcc cattgggttt ttaggaaaac agtgtgaacc |
| 4501 | tccccctttt aatttctggt gttatgagga agaataaagg ggataaaagg ggctaagatg |
| 4561 | gactcatgtt tagctaagtt ctgacttgta tccagcatgc tggagaccaa agctgccgcc |
| 4621 | ttactgctat ttttaagtgc cctcttttca gtcatttgca taattgcgtc catagagctg |
| 4681 | catatgttgt gaataaattc tcactcattt caactttgaa taatttgact gtcttgataa |
| 4741 | ttggttcctc ccaaagactc ttctgcaact cccattcatg cccaccaggc ctcagactcc |
| 4801 | ctcttttccc cgccctgcac tattggagcc ctgggttttg tgggagtgct cagcaccgtg |
| 4861 | agtcttactg tttgatcgga cagttagcaa gatcagatcc tttttgctta ttttctatca |
| 4921 | ctttggaggg ttttctgtag caaaatcagt gaccaatgaa gtaacttaaa ttcctattga |
| 4981 | agaaaaaaaa taataaacca cttgattt |

An exemplary human miR-211 nucleic acid sequence is set forth below (SEQ ID NO: 83; GenBank Accession No: NR_029624.1, Version 1, incorporated herein by reference):

| | |
|---|---|
| 1 | tcacctggcc atgtgacttg tgggcttccc tttgtcatcc ttcgcctagg gctctgagca |
| 61 | gggcagggac agcaaagggg tgctcagttg tcacttccca cagcacggag |

An exemplary human CD5L amino acid sequence is set forth below (SEQ ID NO: 84; GenBank Accession No: AAQ88858.1, Version 1, incorporated herein by reference):

| | |
|---|---|
| 1 | mallfslila ictrpgflas psgvrlvggl hrcegrveve qkgqwgtvcd dgwdikdvav |
| 61 | lcrelgcgaa sgtpsgilye ppaekeqkvl iqsvsctgte dtlaqceqee vydcshdeda |
| 121 | gascenpess fspvpegvrl adgpghckgr vevkhqnqwy tvcqtgwslr aakvvcrqlg |
| 181 | cgravltqkr cnkhaygrkp iwlsqmscsg reatlqdcps gpwgkntcnh dedtwveced |
| 241 | pfdlrlvggd nlcsgrlevl hkgvwgsvcd dnwgekedqv vckqlgcgks lspsfrdrkc |
| 301 | ygpgvgriwl dnvrcsgeeq sleqcqhrfw gfhdcthqed vavicsv |

An exemplary human CD5L nucleic acid sequence is set forth below (SEQ ID NO: 85; GenBank Accession No: NM_005894.2, Version 2, incorporated herein by reference):

| | |
|---|---|
| 1 | gatcatctga taatgctttg cctgcactca ggacctgtct ttgtccctcc tcttaacata |
| 61 | cttgcagcta aaactaaata ttgctgcttg gggacctcct tctagcctta aatttcagct |
| 121 | catcaccttc acctgccttg gtcatggctc tgctattctc cttgatcctt gccatttgca |
| 181 | ccagacctgg attcctagcg tctccatctg gagtgcggct ggtggggggc ctccaccgct |
| 241 | gtgaagggcg ggtggaggtg gaacagaaag gccagtgggg caccgtgtgt gatgacggct |
| 301 | gggacattaa ggacgtggct gtgttgtgcc gggagctggg ctgtggagct gccagcggaa |
| 361 | cccctagtgg tattttgtat gagccaccag cagaaaaaga gcaaaaggtc ctcatccaat |
| 421 | cagtcagttg cacaggaaca gaagatacat tggctcagtg tgagcaagaa gaagtttatg |
| 481 | attgttcaca tgatgaagat gctggggcat cgtgtgagaa cccagagagc tctttctccc |
| 541 | cagtcccaga gggtgtcagg ctggctgacg gccctgggca ttgcaaggga cgcgtggaag |
| 601 | tgaagcacca gaaccagtgg tataccgtgt gccagacagg ctggagcctc cgggccgcaa |
| 661 | aggtggtgtg ccggcagctg ggatgtggga gggctgtact gactcaaaaa cgctgcaaca |
| 721 | agcatgccta tggccgaaaa cccatctggc tgagccagat gtcatgctca ggacgagaag |
| 781 | caacccttca ggattgccct tctgggcctt gggggaagaa cacctgcaac catgatgaag |
| 841 | acacgtgggt cgaatgtgaa gatccctttg acttgagact agtaggagga gacaacctct |
| 901 | gctctgggcg actggaggtg ctgcacaagg gcgtatgggg ctctgtctgt gatgacaact |
| 961 | ggggagaaaa ggaggaccag gtggtatgca agcaactggg ctgtgggaag tccctctctc |
| 1021 | cctccttcag agaccggaaa tgctatggcc ctggggttgg ccgcatctgg ctggataatg |
| 1081 | ttcgttgctc aggggaggag cagtccctgg agcagtgcca gcacagattt tgggggtttc |
| 1141 | acgactgcac ccaccaggaa gatgtggctg tcatctgctc aggatagtat cctggtgttg |
| 1201 | cttgacctgg cccccctggc cccgcctgcc ctctgcttgt tctcctgagc cctgattatc |
| 1261 | ctcatactca ttctggggct caggcttgag ccactactcc ctcatcccct caggagtctg |
| 1321 | aacactgggc ttatgcctta ctctcaggga caagcagccc ccattgctgc ctgtagatgt |
| 1381 | gagctgttga gttccctctt gctggggaag atgagcttcc atgtatcctg tgctcaaccc |
| 1441 | tgaccctttg acactggttc tggcctttcc tgccttttct caagctgcct ggaatcctca |
| 1501 | aacctgtcac tttggtcaga tgtgcagacc attactaagg tctatgtctg caaacattac |
| 1561 | taatctaggt cctattacta atctatgtct gcaaacatta aaggaatgaa acaatgaaag |
| 1621 | gaacatttga aagaaaatgt gggtagacaa tttcttgcaa cttgggggaa agtttagaat |
| 1681 | tcttttgatt ggactacttt tttttttttt cctcaagctt caggtgacca caatagcaac |
| 1741 | acctccctat tctgttattt cttagtgtag gtagacaatt ctttcaggag cagagcagcg |
| 1801 | tcctataatc ctagaccttt tcatgacgtg taaaaaatga tgtttcatcc tctgattgcc |
| 1861 | ccaataaaaa tctttgttgt ccatccctat acaacctgcc aacatggttg acatttaatg |
| 1921 | agaggaatgt caaaaataca ttttacttta ttcaaagaaa aatatattgg ttactgggaa |
| 1981 | aaggtcaaga aagaggcaga aagagatcag ggagggctaa agttgtgtct tatgccaagc |
| 2041 | gaaagtgaaa aatatcattt tcactttatc aactgagact ttggggcctg taagcttgag |
| 2101 | gcaagacaga aataagagaa tcaagacttg attgtaaaaa ttgacaactt tagattctga |
| 2161 | ggctaggctg agtacttatt atacggctac atttacacat ttacacttat ctaataaatc |
| 2221 | agatttcaca gtctcaaaaa aaaaaaaa |

An exemplary human IL12RB2 amino acid sequence is set forth below (SEQ ID NO: 86; GenBank Accession No: AAI43250.1, Version 1, incorporated herein by reference):

| | |
|---|---|
| 1 | mahtfrgcsl afmfiitwll ikakidackr gdvtvkpshv illgstvnit cslkprqgcf |
| 61 | hysrrnklil ykfdrrinfh hghslnsqvt glplgttlfv cklacinsde iqicgaeifv |
| 121 | gvapeqpqnl sciqkgeqgt vactwergrd thlyteytlq lsgpknltwq kqckdiycdy |
| 181 | ldfginltpe spesnftakv tavnslgsss slpstftfld ivrplppwdi rikfqkasvs |
| 241 | rctlywrdeg lvllnrlryr psnsrlwnmv nvtkakgrhd lldlkpftey efqissklhl |
| 301 | ykgswsdwse slraqtpeee ptgmldvwym krhidysrqq islfwknlsv seargkilhy |
| 361 | qvtlqeltgg kamtqnitgh tswttviprt gnwavavsaa nskgsslptr inimnlceag |
| 421 | llaprhvsan segmdnilvt wqpprkdpsa vqeyvvewre lhpggdtqvp lnwlrsrpyn |
| 481 | vsaliseipy rvsqnshpin slqprvtyvl wmtaltaage sshgnerefc lqgkanwmaf |
| 541 | vapsiciaii mvgifsthyf qqkvfvllaa lrpqwcsrei pdpanstcak kypiaeektq |
| 601 | lpldrllidw ptpedpeplv isevlhqvtp vfrhppcsnw pgrekgiggh qasekdmmhs |
| 661 | assppppral qaesrqlvdl ykvlesrgsd pkpenpacpw tvlpagdlpt hdgylpsnid |
| 721 | dlpsheapla dsleelepqh islsvfpsss lhpltfscgd kltldqlkmr cdslml |

An exemplary human IL12RB2 nucleic acid sequence is set forth below (SEQ ID NO: 87; GenBank Accession No: NM_001559.2, Version 2, incorporated herein by reference):

| | |
|---|---|
| 1 | tgcagagcac agagaaagga catctgcgag gaaagttccc tgatggctgt caacaaagtg |
| 61 | ccacgtctct atggctgtga acgctgagca cacgatttta tcgcgcctat catatcttgg |
| 121 | tgcataaacg cacctcacct cggtcaaccc ttgctccgtc ttatgagaca ggctttatta |
| 181 | tccgcatttt atatgagggg aaactgacgg tggagagaga attatcttgc tcaaggcgac |
| 241 | acagcagagc ccacaggtgg cagaatccca cccgagcccg cttcgacccg cggggtggaa |
| 301 | accacgggcg cccgcccggc tgcgcttcca gagctgaact gagaagcgag tcctctccgc |
| 361 | cctgcggcca ccgcccagcc ccgacccccg ccccggcccg atcctcactc gccgccagct |
| 421 | ccccgcgccc accccggagt tggtggcgca gaggcgggag gcggaggcgg gagggcgggc |
| 481 | gctggcaccg ggaacgcccg agcgccggca gagagcgcgg agagcgcgac acgtgcggcc |
| 541 | cagagcaccg gggccacccg gtccccgcag gcccgggacc gcgcccgctg gcaggcgaca |
| 601 | cgtggaagaa tacggagttc tataccagag ttgattgttg atggcacata cttttagagg |
| 661 | atgctcattg gcatttatgt ttataatcac gtggctgttg attaaagcaa aaatagatgc |
| 721 | gtgcaagaga ggcgatgtga ctgtgaagcc ttcccatgta attttacttg gatccactgt |
| 781 | caatattaca tgctctttga agcccagaca aggctgcttt cactattcca gacgtaacaa |
| 841 | gttaatcctg tacaagtttg acagaagaat caattttcac catggccact ccctcaattc |
| 901 | tcaagtcaca ggtcttcccc ttggtacaac cttgtttgtc tgcaaactgg cctgtatcaa |
| 961 | tagtgatgaa attcaaatat gtggagcaga gatcttcgtt ggtgttgctc cagaacagcc |
| 1021 | tcaaaattta tcctgcatac agaagggaga acaggggact gtggcctgca cctgggaaag |
| 1081 | aggacgagac acccacttat acactgagta tactctacag ctaagtggac caaaaaattt |
| 1141 | aacctggcag aagcaatgta aagacattta ttgtgactat ttggactttg gaatcaacct |
| 1201 | cacccctgaa tcacctgaat ccaatttcac agccaaggtt actgctgtca atagtcttgg |
| 1261 | aagctcctct tcacttccat ccacattcac attcttggac atagtgaggc ctcttcctcc |
| 1321 | gtgggacatt agaatcaaat ttcaaaaggc ttctgtgagc agatgtaccc tttattggag |
| 1381 | agatgaggga ctggtactgc ttaatcgact cagatatcgg cccagtaaca gcaggctctg |
| 1441 | gaatatggtt aatgttacaa aggccaaagg aagacatgat ttgctggatc tgaaaccatt |
| 1501 | tacagaatat gaatttcaga tttcctctaa gctacatctt tataagggaa gttggagtga |
| 1561 | ttggagtgaa tcattgagag cacaaacacc agaagaagag cctactggga tgttagatgt |
| 1621 | ctggtacatg aaacggcaca ttgactacag tagacaacag atttctcttt tctggaagaa |
| 1681 | tctgagtgtc tcagaggcaa gaggaaaaat tctccactat caggtgacct tgcaggagct |
| 1741 | gacaggaggg aaagccatga cacagaacat cacaggacac acctcctgga ccacagtcat |
| 1801 | tcctagaacc ggaaattggg ctgtggctgt gtctgcagca aattcaaaag gcagttctct |
| 1861 | gcccactcgt attaacataa tgaacctgtg tgaggcaggg ttgctggctc ctcgccaggt |
| 1921 | ctctgcaaac tcagagggca tggacaacat tctggtgact tggcagcctc ccaggaaaga |
| 1981 | tccctctgct gttcaggagt acgtggtgga atggagagag ctccatccag ggggtgacac |
| 2041 | acaggtccct ctaaactggc tacggagtcg accctacaat gtgtctgctc tgatttcaga |
| 2101 | gaacataaaa tcctacatct gttatgaaat ccgtgtgtat gcactctcag gggatcaagg |
| 2161 | aggatgcagc tccatcctgg gtaactctaa gcacaaagca ccactgagtg gcccccacat |
| 2221 | taatgccatc acagaggaaa aggggagcat tttaatttca tggaacagca ttccagtcca |
| 2281 | ggagcaaatg ggctgcctcc tccattatag gatatactgg aaggaacggg actccaactc |
| 2341 | ccagcctcag ctctgtgaaa ttccctacag agtctcccaa aattcacatc caataaacag |
| 2401 | cctgcagccc cgagtgacat atgtcctgtg gatgacagct ctgacagctg ctggtgaaag |
| 2461 | ttcccacgga aatgagaggg aattttgtct gcaaggtaaa gccaattgga tggcgtttgt |
| 2521 | ggcaccaagc atttgcattg ctatcatcat ggtgggcatt ttctcaacgc attacttcca |
| 2581 | gcaaaaggtg tttgttctcc tagcagccct cagacctcag tggtgtagca gagaaattcc |
| 2641 | agatccagca aatagcactt gcgctaagaa atatcccatt gcagaggaga agacacagct |
| 2701 | gcccttggac aggctcctga tagactggcc cacgcctgaa gatcctgaac cgctggtcat |
| 2761 | cagtgaagtc cttcatcaag tgaccccagt tttcagacat cccccctgct ccaactggcc |
| 2821 | acaaagggaa aaaggaatcc aaggtcatca ggcctctgag aaagacatga tgcacagtgc |
| 2881 | ctcaagccca ccacctccaa gagctctcca agctgagagc agacaactgg tggatctgta |
| 2941 | caaggtgctg gagagcaggg gctccgaccc aaagcccgaa aacccagcct gtccctggac |
| 3001 | ggtgctccca gcaggtgacc ttcccaccca tgatggctac ttaccctcca acatagatga |
| 3061 | cctcccctca catgaggcac ctctcgctga ctctctggaa gaactggagc ctcagcacat |
| 3121 | ctccctttct gttttcccct caagttctct tcacccactc accttctcct gtggtgataa |
| 3181 | gctgactctg gatcagttaa agatgaggtg tgactccctc atgctctgag tggtgaggct |
| 3241 | tcaagcctta aagtcagtgt gccctcaacc agcacagcct gccccaattc ccccagcccc |
| 3301 | tgctccagca gctgtcatct ctgggtgcca ccatcggtct ggctgcagct agaggacagg |
| 3361 | caagccagct ctgggggagt cttaggaact gggagttggt cttcactcag atgcctcatc |
| 3421 | ttgcctttcc cagggcctta aaattacatc cttcactgtg tggacctaga gactccaact |
| 3481 | tgaattccta gtaactttct tggtatgctg gccagaaagg gaaatgagga ggagagtaga |
| 3541 | aaccacagct cttagtagta atggcataca gtctagagga ccattcatgc aatgactatt |
| 3601 | tctaaagcac ctgctacaca gcaggctgta cacagcagat cagtactgtt caacagaact |
| 3661 | tcctgagatg atggaaatgt tctacctctg cactcactgt ccagtacatt agacactagg |
| 3721 | cacattggct gttaatcact tggaatgtgt ttagcttgac tgaggaatta aattttgatt |
| 3781 | gtaaatttaa atcgccacac atggctagtg gctactgtat tggagtgcac agctctagat |
| 3841 | ggctcctaga ttattgagag ccttcaaaac aaatcaacct agttctatag atgaagacat |
| 3901 | aaaagacact ggtaaacacc aaggtaaaag ggcccccaag gtggtcatga ctggtctcat |
| 3961 | ttgcagaagt ctaagaatgt acctttttct ggccgggcgt ggtagctcat gcctgtaatc |
| 4021 | ccagcacttt gggaggctga |

An exemplary human FAIM3 amino acid sequence is set forth below (SEQ ID NO: 88; GenBank Accession No: EAW93517.1, Version 1, incorporated herein by reference):

| | |
|---|---|
| 1 | mdfwlwplyf lpvsgalril pevkvegelg gsvtikcplp emhvriylcr emagsgtcgt |
| 61 | vvsttnfika eykgrvtlkq yprknlflve vtqltesdsg vyacgagmnt drgktqkvtl |
| 121 | nvhseyepsw eeqpmpetpk wfhlpylfqm payassskfv trvttpaqrg kvppvhhssp |
| 181 | ttqithrprv srassvagdk prtflpstta skisalegll kpqtpsynhh trlhrqrald |
| 241 | ygsqsgregq gfhiliptil glfllallgl vvkraverrk alsrrarrla vrmralessq |
| 301 | rprgsprprs qnniysacpr rargadaagt geapvpgpga plppaplqvs espwlhapsl |
| 361 | ktsceyvsly hqpaammeds dsddyinvpa |

An exemplary human FAIM3 nucleic acid sequence is set forth below (SEQ ID NO: 89; GenBank Accession No: NM_005449.4, Version 4, incorporated herein by reference):

| | |
|---|---|
| 1 | agcctgagaa tagttagcaa acaagggagg ttgtcatttc ctcatcgtca agctttgttc |
| 61 | ctcgtggggg ctagaaatct ctttccagtt ccagattgtg aagggttcct gagtaagcag |
| 121 | cgtgtctcca tccccctctc taggggctct tggatggacc ttgcactcta gaagggacaa |
| 181 | tggacttctg gctttggcca ctttacttcc tgccagtatc gggggccctg aggatcctcc |
| 241 | cagaagtaaa ggtagagggg gagctgggcg gatcagttac catcaagtgc ccacttcctg |
| 301 | aaatgcatgt gaggatatat ctgtgccggg agatggctgg atctggaaca tgtggtaccg |
| 361 | tggtatccac caccaacttc atcaaggcag aatacaaggg ccgagttact ctgaagcaat |
| 421 | acccacgcaa gaatctgttc ctagtggagg taacacagct gacagaaagt gacagcggag |
| 481 | tctatgcctg cggagcgggc atgaacacag accggggaaa gacccagaaa gtcaccctga |
| 541 | atgtccacag tgaatacgag ccatcatggg aagagcagcc aatgcctgag actccaaaat |
| 601 | ggtttcatct gccctatttg ttccagatgc ctgcatatgc cagttcttcc aaattcgtaa |
| 661 | ccagagttac cacaccagct caaaggggca aggtccctcc agttcaccac tcctccccca |
| 721 | ccacccaaat cacccaccgc cctcgagtgt ccagagcatc ttcagtagca ggtgacaagc |
| 781 | cccgaacctt cctgccatcc actacagcct caaaaatctc agctctggag gggctgctca |
| 841 | agccccagac gcccagctac aaccaccaca ccaggctgca caggcagaga gcactggact |
| 901 | atggctcaca gtctgggagg gaaggccaag gatttcacat cctgatcccg accatcctgg |
| 961 | gccttttcct gctggcactt ctggggctgg tggtgaaaag ggccgttgaa aggaggaaag |
| 1021 | ccctctccag gcgggcccgc cgactggccg tgaggatgcg cgccctggag agctcccaga |
| 1081 | ggccccgcgg gtcgccgcga ccgcgctccc aaaacaacat ctacagcgcc tgcccgcggc |
| 1141 | gcgctcgtgg agcggacgct gcaggcacag gggaggcccc cgttcccggc cccggagcgc |
| 1201 | cgttgccccc cgccccgctg caggtgtctg aatctccctg gctccatgcc ccatctctga |
| 1261 | agaccagctg tgaatacgtg agcctctacc accagcctgc cgccatgatg gaggacagtg |
| 1321 | attcagatga ctacatcaat gttcctgcct gacaactccc cagctatccc ccaaccccag |
| 1381 | gctcggactg tggtgccaag gagtctcatc tatctgctga tgtccaatac ctgcttcatg |
| 1441 | tgttctcaga gccctcatca cttcccatgc cccatctcga ctcccatccc catctatctg |
| 1501 | tgccctgagc atggctctgc ccccaggtcg tcttgcacac cttggcagcc ccctgtagtt |
| 1561 | gacaggtaag ctgtaggcat gtagagcaat tgtcccaatg ccacttgctt cctttccaag |
| 1621 | ccgtcgaaca gactgtggga tttgcagagt gtttcttcca tgtctttgac cacagggttg |
| 1681 | ttgctgccca ggctctagat cacatggcat caggctgggg cagaggcata gctattgtct |
| 1741 | cgggcatcct tcccagggtt gggtcttaca caaatagaag gctcttgctc tgagttatgt |
| 1801 | gacatgcctc agccccatgg actaagcagg ggtctggtat aaaaacactc ctggaaacgc |
| 1861 | ctttgccctg atccaaatgt tagcacttgc tagtgaacgt ctacttatct caagttctat |
| 1921 | gctaaaggca atttatcttg atgtgatgat aaaccaaact tattagcaag atatgcatat |
| 1981 | atatccataa attctcttta ctctgtctcc atcacttgat gcacataagt gccctgacct |
| 2041 | cagcatctcc cctctaaaaa aaaaaaaaaa aaagtatctc tttatctttc ttccatagcc |
| 2101 | tgacactgat atttgtgcac ttaccttaac tttggtctat tttattcatc caaaaccatt |
| 2161 | acatttcttg gttttcacaa atgttcccca tttcttagcc agttccagac aatgtatagc |
| 2221 | aagcagggga aggaaagcag tcaggagttc ctgggtggcc acggctctgc aatagcactt |
| 2281 | atgtcatgga agtgatatcc cacctcctac atatactctt tgcctaggtt tttggaacaa |
| 2341 | ggttatagtc agacactgta tctttagatt gatgtcgacc acaaagttca gccagagctt |
| 2401 | gaggctagat gcacagcctt gctattggga agaaggcctt ttctagctgt acaacacagt |
| 2461 | ctcactgggc attcatccag aaatagagaa gaaagtctgc cagacttgag ttatgttgtc |
| 2521 | ttttattagc agggaatgtc atcacagatt ggatagtaca tccaggtgca atgtcaccat |
| 2581 | cagcaaggtc agcttgacac tcaagtggaa gattagggaa gaatgactag gataaaaaaa |
| 2641 | aaaggagggc accaagggaa agggatgatg gggtgagctg gcgagtgtgg gtgggaaatg |
| 2701 | aaatgtttat tgaggatctg ctttgtgctg ggcactttaa tccacatttt atcgtttact |
| 2761 | tttcaaacag atgcacctta cccccacccc aatgctctgt ccctgcagat atcagaagac |
| 2821 | agtgtgattt tcatgctctg aagttcagtt ttacatccaa gcatccctct ctgtttttta |
| 2881 | acaatccaaa gacaggccaa aaaaagcacc acagtttatt aagtacttac taagcaccca |
| 2941 | tccactgccc cacactgtgg caaggattgt gaggggtaaa gaagcatggg gcacaatatt |
| 3001 | ctgctgcctt catgtaactt acagtctcac aaataaatag aacttcagtt gaaatactga |
| 3061 | cattaattaa atagagttgt aataaaaaaa aaaaaaaaaa aaaaaaaaa |

An exemplary human PTCRA amino acid sequence is set forth below (SEQ ID NO: 90; GenBank Accession No: AAI53830.1, Version 1, incorporated herein by reference):

| | |
|---|---|
| 1 | magtwlllll algcpalptg vggtpfpsla ppimllvdgk qqmvvvclvl dvappgldsp |
| 61 | iwfsagngsa ldaftygpsp atdgtwtnla hlslpseela sweplichtg pgaeghsrst |
| 121 | qpmhlsgeas tartcpqepl rgtpggalwl gvlrlllfkl llfdllltcs clcdpagplp |
| 181 | spatttrlra lgshrlhpat etggreatss prpqprdrrw gdtppgrkpg spvwgegsyl |
| 241 | ssyptcpaqa wcsrsalrap ssslgaffag dlppplqaga a |

An exemplary human PTCRA nucleic acid sequence is set forth below (SEQ ID NO: 91; GenBank Accession No: NM_001243168.1, Version 1, incorporated herein by reference):

| | |
|---|---|
| 1 | tagaaggcag tcttgtgggt gcctcctccc ccagccgcaa ctcaggtctg cagctgggtc |
| 61 | ctgcctcctt ccgagtgggc catggccggt acatggctgc tacttctcct ggcccttggg |
| 121 | tgtccagccc tacccacagg tgtgggcggc acaccctttc cttctctggc cccaccaatc |
| 181 | atgctgctgg tggatggaaa gcagcagatg gtggtggtct gcctggtcct tgatgttgca |
| 241 | ccccctggcc ttgacagccc catctggttc tcagccggca atggcagtgc actggatgcc |
| 301 | ttcacctatg gcccttcccc agcaacggat ggcacctgga ccaacttggc ccatctctcc |
| 361 | ctgccttctg aggagctggc atcctgggag cctttggtct gccacactgg gcctggggct |
| 421 | gagggtcaca gcaggagtac acagcccatg catctgtcag gagaggcttc tacagccagg |
| 481 | acctgccccc aggagcctct cagggggggc tgcgggctcc tgcgggctcc tgagcggttc |
| 541 | ctcctcgcag ggacaccggg tggggcgctg tggctggggg tcctgcggct gctgctcttc |
| 601 | aagctgctgc tgtttgacct gctcctgacc tgcagctgcc tgtgcgaccc cgcgggcccg |
| 661 | ctgccttccc ccgcaaccac cacccgcctg cgagccctcg gctcccatcg actgcacccg |
| 721 | gccacggaga ctgggggacg agaggccacc agctcaccca gaccccagcc tcgggaccgc |
| 781 | cgctggggtg acacccctcc gggtcggaag cccgggagcc cagtatgggg ggaagggtct |
| 841 | tacctcagca gttaccccac ttgcccagca caggcctggt gctcaagatc tgccctcagg |
| 901 | gctccttcct ccagtcttgg agcatttttt gcaggtgacc tgcctcctcc tctgcaggct |
| 961 | ggagctgcct gagggcaggg ctctacctcc cctgcgtcac actgtgtgag gctgtgtctc |
| 1021 | tgccatccaa aagggggccc cttgagaatg gtgatccacc cagttacagg ggcatttagg |
| 1081 | gagcagatga ctgagaacat taaaaaagaa cttaaatgac acagcaaaaa aaaaaaaaaa |
| 1141 | aa |

An exemplary human CD2 amino acid sequence is set forth below (SEQ ID NO: 92; GenBank Accession No: AAA51946.1, Version 1, incorporated herein by reference):

| | |
|---|---|
| 1 | msfpckfvas fllifnvssk gavskeitna letwgalgqd inldipsfqm sddiddikwe |
| 61 | ktsdkkkiaq frkeketfke kdtyklfkng tlkikhlktd dqdiykvsiy dtkgknvlek |
| 121 | ifdlkiqery skpkiswtci nttltcevmn gtdpelnlyq dgkhlklsqr vithkwttsl |
| 181 | sakfkctagn kvskessvep vscpekgldi yliigicggg sllmvfvall vfyitkrkkq |
| 241 | rsrrndeele trahrvatee rgrkphqipa stpqnpatsq hpppppghrs qapshrpppp |
| 301 | ghrvqhqpqk rppapsgtqv hqqkgpplpr prvqpkppmg qqkthcplpl ikkdrnclfq |

An exemplary human CD2 nucleic acid sequence is set forth below (SEQ ID NO: 93; GenBank Accession No: NM_001328609.1, Version 1, incorporated herein by reference):

| | |
|---|---|
| 1 | agtctcactt cagttccttt tgcatgaaga gctcagaatc aaaagaggaa accaacccct |
| 61 | aagatgagct ttccatgtaa atttgtagcc agcttccttc tgattttcaa tgtttcttcc |
| 121 | aaaggtgcag tctccaaaga gattacgaat gccttggaaa cctggggtgc cttgggtcag |
| 181 | gacatcaact tggacattcc tagttttcaa atgagtgatg atattgacga tataaaatgg |
| 241 | gaaaaaactt cagacaagaa aaagattgca caattcagaa aagagaaaga gactttcaag |
| 301 | gaaaaagata catataagct atttaaaaat ggaactctga aaattaagca tctgaagacc |
| 361 | gatgatcagg atatctacaa ggtatcaata tatgatacaa aaggaaaaaa tgtgttggaa |
| 421 | aaaatatttg atttgaagat tcaagagagg gtctcaaaac caaagatctc ctggacttgt |
| 481 | atcaacacaa ccctgacctg tgaggtaatg aatggaactg accccgaatt aaacctgtat |
| 541 | caagatggga aacatctaaa actttctcag agggtcatca cacacaagtg gaccaccagc |
| 601 | ctgagtgcaa aattcaagtg cacagcaggg aacaaagtca gcaaggaatc cagtgtcgag |
| 661 | cctgtcagct gtccaggagg cagcatcctt ggccagagta atgggctctc tgcctggacc |
| 721 | cctcccagcc atcccacttc tcttcctttt gcagagaaag gtctggacat ctatctcatc |
| 781 | attggcatat gtggaggagg cagcctcttg atggtctttg tggcactgct cgttttctat |
| 841 | atcaccaaaa ggaaaaaaca gaggagtcgg agaaatgatg aggagctgga gacaagagcc |
| 901 | cacagagtag ctactgaaga aaggggccgg aagccccacc aaattccagc ttcaacccct |
| 961 | cagaatccag caacttccca acatcctcct ccaccacctg gtcatcgttc ccaggcacct |
| 1021 | agtcatcgtc ccccgcctcc tggacaccgt gttcagcacc agcctcagaa gaggcctcct |
| 1081 | gctccgtcgg gcacacaagt tcaccagcag aaaggcccgc ccctccccag acctcgagtt |
| 1141 | cagccaaaac ctccccatgg ggcagcagaa aactcattgt ccccttcctc taattaaaaa |
| 1201 | agatagaaac tgtctttttc aataaaaagc actgtggatt tctgccctcc tgatgtgcat |
| 1261 | atccgtactt ccatgaggtg ttttctgtgt gcagaacatt gtcacctcct gaggctgtgg |
| 1321 | gccacagcca cctctgcatc ttcgaactca gccatgtggt caacatctgg agtttttggt |
| 1381 | ctcctcagag agctccatca caccagtaag gagaagcaat ataagtgtga ttgcaagaat |
| 1441 | ggtagaggac cgagcacaga aatcttagag atttcttgtc ccctctcagg tcatgtgtag |
| 1501 | atgcgataaa tcaagtgatt ggtgtgcctg ggtctcacta caagcagcct atctgcttaa |
| 1561 | gagactctgg agtttcttat gtgccctggt ggacacttgc ccaccatcct gtgagtaaaa |
| 1621 | gtgaaataaa agctttgact agaaaaaaaa aaaaaaaaaa |

An exemplary human CD6 amino acid sequence is set forth below (SEQ ID NO: 94; GenBank Accession No: AAH33755.1, Version 1, incorporated herein by reference):

| | |
|---|---|
| 1 | mwlffgitgl ltaalsghps pappdqlnts saeselwepg erlpvrltng ssscsgtvev |
| 61 | rleaswepac galwdsraae avcralgcgg aeaasqlapp tpelppppaa gntsvaanat |
| 121 | lagapallcs gaewrlcevv ehacrsdgrr arvtcaenra lrlvdgggac agrvemlehg |
| 181 | ewgsvcddtw dledahvvcr qlgcgwavqa lpglhftpgr gpihrdqvnc sgaeaylwdc |
| 241 | pglpgqhycg hkedagavcs ehqswrltgg adrcegqvev hfrgvwntvc dsewypseak |
| 301 | vlcqslgcgt averpkglph slsgrmyysc ngeeltlsnc swrfnnsnlc sqslaarvlc |
| 361 | sasrslhnls tpevpasvqt vtiessvtvk ienkesrelm llipsivlgi lllgslifia |
| 421 | fillrikgky alpvmvnhqh lpttipagsn syqpvpitip kevfmlpiqv qapppedsds |
| 481 | gsdsdyehyd fsaqppvalt tfynsqrhrv tdeevqqsrf qmppleegle elhashipta |
| 541 | npghcitdpp slgpqyhprs nsesstssge dycnspkskl ppwnpqvfss erssfleqpp |
| 601 | nlelagtqpa fsgppaddss stssgewyqn fqprllgwl |

An exemplary human CD6 nucleic acid sequence is set forth below (SEQ ID NO: 95; GenBank Accession No: NM_006725.4, Version 4, incorporated herein by reference):

| | |
|---|---|
| 1 | gcagaccaaa accacaagca gaacaagcag gcgtgagaca ctcacaggtt gggtttgatc |
| 61 | gcatgcgtgt cggagaggag agagcagaga gagacacagg aacaagaaca gcaaagggta |
| 121 | gagcagacct gcgccagggg cgcacaacgg ccgtgtccac ctcccggccc caagatggtg |
| 181 | cttcccacag gcagccacgc gtagcagcca gagacagctc cagacatgtg gctcttcttc |
| 241 | gggatcactg gattgctgac ggcagccctc tcaggtcatc catctccagc cccacctgac |
| 301 | cagctcaaca ccagcagtgc agagagtgag ctctgggagc caggggagcg gcttccggtc |
| 361 | cgtctgacaa acgggagcag cagctgcagc gggacggtgg aggtgcggct cgaggcgtcc |
| 421 | tgggagcccg cgtgcggggc gctctgggac agccgcgccg ccgaggccgt gtgccgagca |
| 481 | ctgggctgcg gcggggcgga ggccgcctct cagctcgccc cgccgacccc tgagctgccg |
| 541 | cccccgcctg cagccgggaa caccagcgta gcagctaatg ccactctggc cggggcgccc |
| 601 | gccctcctgt gcagcggcgc cgagtggcgg ctctgcgagg tggtggagca cgcgtgccgc |
| 661 | agcgacggga ggcgggcccg tgtcacctgt gcagagaacc gcgcgctgcg cctggtggac |
| 721 | ggtggcggcg cctgcgccgg ccgcgtggag atgctggagc atggcgagtg gggatcagtg |
| 781 | tgcgatgaca cttgggacct ggaggacgcc cacgtggtgt gcaggcaact gggctgcggc |
| 841 | tgggcagtcc aggccctgcc cggcttgcac ttcacgcccg gccgcgggcc tatccaccgg |
| 901 | gaccaggtga actgctcggg ggccgaagct tacctgtggg actgcccggg gctgccagga |
| 961 | cagcactact gcggccacaa agaggacgcg ggcgcggtgt gctcagagca ccagtcctgg |
| 1021 | cgcctgacag ggggcgctga ccgctgcgag gggcaggtgg aggtacactt ccgaggggtc |
| 1081 | tggaacacag tgtgtgacag tgagtggtac ccatcggagg ccaaggtgct ctgccagtcc |
| 1141 | ttgggctgtg gaactgcggt tgagaggccc aaggggctgc cccactcctt gtccggcagg |
| 1201 | atgtactact catgcaatgg ggaggagctc accctctcca actgctcctg gcggttcaac |
| 1261 | aactccaacc tctgcagcca gtcgctggca gccagggtcc tctgctcagc ttcccggagt |
| 1321 | ttgcacaatc tgtccactcc cgaagtccct gcaagtgttc agacagtcac tatagaatct |
| 1381 | tctgtgacag tgaaaataga gaacaaggaa tctcgggagc taatgctcct catcccctcc |
| 1441 | atcgttctgg gaattctcct ccttggctcc ctcatcttca tagccttcat cctcttgaga |
| 1501 | attaaaggaa aatatgccct ccccgtaatg gtgaaccacc agcacctacc caccaccatc |
| 1561 | ccggcaggga gcaatagcta tcaaccggtc cccatcacca tccccaaaga agttttcatg |
| 1621 | ctgcccatcc aggtccaggc cccgccccct gaggactcag actctggctc ggactcagac |
| 1681 | tatgagcact atgacttcag cgcccagcct cctgtggccc tgaccacctt ctacaattcc |
| 1741 | cagcggcatc gggtcacaga tgaggaggtc cagcaaagca ggttccagat gccacccttg |
| 1801 | gaggaaggac ttgaagagtt gcatgcctcc cacatcccaa ctgccaaccc tggacactgc |
| 1861 | attacagacc cgccatccct gggccctcag tatcacccga ggagcaacag tgagtcgagc |
| 1921 | acctcttcag gggaggatta ctgcaatagt cccaaaagca agctgcctcc atggaacccc |
| 1981 | caggtgtttt cttcagagag gagttccttc ctggagcagc ccccaaactt ggagctggcc |
| 2041 | ggcacccagc cagccttttc agcagggccc ccggctgatg acagctccag cacctcatcc |
| 2101 | ggggagtggt accagaactt ccagccacca ccccagcccc cttcggagga gcagtttggc |
| 2161 | tgtccagggt cccccagccc tcagcctgac tccaccgaca acgatgacta cgatgacatc |
| 2221 | agcgcagcct aggccggggc cagccgaggc tcctggggtg gctctgaccc tctggcctcc |
| 2281 | tgctctacct actccctttc ccctttccca ccctcccagc tcacctcccc atggagctga |
| 2341 | gaggcctccc ttggagagat ggaaggaaac gttatacctt gtacccctcg gtctccatcc |
| 2401 | atcaagccaa acctgctgcc acagccctcc cccggcccca gatagcagcc ccagggagga |
| 2461 | tgctgcctcc aagaggtgtg agccctctgt ctcggggatg aacaagcaga gtctgggcta |
| 2521 | cctcttgaca gctggtggag gggagttggg gagctggact ggatgactct ggaggcccct |
| 2581 | tccaaacctc aagtgtccgg cgctttgatt gcctgagttt ctgacacttc agggcccaga |
| 2641 | ggtcctgcga ggggcagaac tggaccccca tgccagtgct gctgcaggag ggcccatata |
| 2701 | ctagggtctg ctgagctgtt gtcactgatc ggtgggcgct gggggggtag ggtagcacac |
| 2761 | cagctgtccc aggctttgct ccgggcggta actgcacttg ggcagggaat atagccttcc |
| 2821 | tgggcacaac tagctgacaa tgacaggttg actgtgtacc cccaaccaag gagctggggc |
| 2881 | ccaaggccag tcctgcccca gagacactcc aagtccgcca ggggcacaga ccagttctgc |
| 2941 | agtgactgtc cctggacaat gggtctttat tctgagtttc ctatggttta caaagagggc |
| 3001 | cccagcccag ccccaccaca gatcccagag ataggggccc agtctccatg ggggcaagga |
| 3061 | gcatagagat gttttccagg aaggggctca gaagctgcac taggccccga gtccccatgt |
| 3121 | gtctccttga attgatgagg atgctcctgg gagggatgcg tgactatgtg gtgttgcacc |
| 3181 | cggggctgca aacgtctccg tgcagccccc agagagaggc ccatgggctc agaccaggct |
| 3241 | ttgttgtcct gctctgagta tcctgagatt aaactgaatt gctgaatgaa aaaaaaaaaa |
| 3301 | aaaaaaaaa |

An exemplary human CXCL13 amino acid sequence is set forth below (SEQ ID NO: 96; GenBank Accession No: AAH12589.1, Version 1, incorporated herein by reference):

| | |
|---|---|
| 1 | mkfistslll mllvsslspv qgvlevyyts lrcrcvqess vfiprrfidr iqilprgngc |
| 61 | prkeiivwkk nksivcvdpq aewiqrmmev lrkrssstlp vpvfkrkip |

An exemplary human CXCL13 nucleic acid sequence is set forth below (SEQ ID NO: 97; GenBank Accession No: NM_006419.2, Version 2, incorporated herein by reference):

| | |
|---|---|
| 1 | gagaagatgt ttgaaaaaac tgactctgct aatgagcctg gactcagagc tcaagtctga |
| 61 | actctacctc cagacagaat gaagttcatc tcgacatctc tgcttctcat gctgctggtc |
| 121 | agcagcctct ctccagtcca aggtgttctg gaggtctatt acacaagctt gaggtgtaga |
| 181 | tgtgtccaag agagctcagt ctttatccct agacgcttca ttgatcgaat tcaaatcttg |
| 241 | ccccgtggga atggttgtcc aagaaaagaa atcatagtct ggaagaagaa caagtcaatt |
| 301 | gtgtgtgtgg accctcaagc tgaatggata caaagaatga tggaagtatt gagaaaaaga |
| 361 | agttcttcaa ctctaccagt tccagtgttt aagagaaaga ttccctgatg ctgatatttc |
| 421 | cactaagaac acctgcattc ttcccttatc cctgctctgg attttagttt tgtgcttagt |
| 481 | taaatctttt ccaggaaaaa gaacttcccc atacaaataa gcatgagact atgtaaaaat |
| 541 | aaccttgcag aagctgatgg ggcaaactca agcttcttca ctcacagcac cctatataca |
| 601 | cttggagttt gcattcttat tcatcaggga ggaaagtttc tttgaaaata gttattcagt |
| 661 | tataagtaat acaggattat tttgattata tacttgttgt ttaatgttta aaatttctta |
| 721 | gaaaacaatg gaatgagaat ttaagcctca aatttgaaca tgtggcttga attaagaaga |
| 781 | aaattatggc atatattaaa agcaggcttc tatgaaagac tcaaaaagct gcctgggagg |
| 841 | cagatggaac ttgagcctgt caagaggcaa aggaatccat gtagtagata tcctctgctt |
| 901 | aaaaactcac tacggaggag aattaagtcc tacttttaaa gaatttcttt ataaaattta |
| 961 | ctgtctaaga ttaatagcat tcgaagatcc ccagacttca tagaatactc agggaaagca |
| 1021 | tttaaagggt gatgtacaca tgtatccttt cacacatttg ccttgacaaa cttctttcac |
| 1081 | tcacatcttt ttcactgact ttttttgtgg ggggcggggc cggggggact ctggtatcta |
| 1141 | attctttaat gattcctata aatctaatga cattcaataa agttgagcaa acattttact |
| 1201 | taaaaaaaaa aaaaaaaaa |

An exemplary human CD3D amino acid sequence is set forth below (SEQ ID NO: 98; GenBank Accession No: AEQ93556.1, Version 1, incorporated herein by reference):

| | |
|---|---|
| 1 | mehstflsgl vlatllsqvc qscveldpat vagiivtdvi atlllalgvf cfaghetgrl |
| 61 | sgaadtqall rndqvygplr drddagyshl ggnwarnk |

An exemplary human CD3D nucleic acid sequence is set forth below (SEQ ID NO: 99; GenBank Accession No: BAJ000732.4, Version 4, incorporated herein by reference):

| | |
|---|---|
| 1 | agagaagcag acatcttcta gttcctcccc cactctcctc tttccggtac ctgtgagtca |
| 61 | gctaggggag ggcagctctc acccaggctg atagttcggt gacctggctt tatctactgg |
| 121 | atgagttccg ctgggagatg gaacatagca cgtttctctc tggcctggta ctggctaccc |
| 181 | ttctctcgca agtgagcccc ttcaagatac ctatagagga acttgaggac agagtgtttg |
| 241 | tgaattgcaa taccagcatc acatgggtag agggaacggt gggaacactg ctctcagaca |
| 301 | ttacaagact ggacctggga aaacgcatcc tggacccacg aggaatatat aggtgtaatg |
| 361 | ggacagatat atacaaggac aaagaatcta ccgtgcaagt tcattatcga atgtgccaga |
| 421 | gctgtgtgga gctggatcca gccaccgtgg ctggcatcat tgtcactgat gtcattgcca |
| 481 | ctctgctcct tgctttggga gtcttctgct ttgctggaca tgagactgga aggctgtctg |
| 541 | gggctgccga cacacaagct ctgttgagga atgaccaggt ctatcagccc ctccgagatc |
| 601 | gagatgatgc tcagtacagc caccttggag gaaactgggc tcggaacaag tgaacctgag |
| 661 | actggtggct tctagaagca gccattacca actgtacctt cccttcttgc tcagccaata |
| 721 | aatatatcct ctttcactca gaaaaaaaaa aaaaaaaaaa aaaaaaaaaa a |

An exemplary human CD3E nucleic acid sequence is set forth below (SEQ ID NO: 101; GenBank Accession No: NM_16130.1, Version 1, incorporated herein by reference):

| | |
|---|---|
| 1 | mqsgthwrvl glcllsvgvw gqdewvvlrg itqtpykvsi sgttviltcp qypgseilwq |
| 61 | hndkniggde ddknigsded hlslkefsel eqsgyyvcyp rgskpedanf ylylrarvce |
| 121 | ncmemdvmsv ativivdici tggllllvyy wsknrkakak pvtrgagagg rqrgqnkerp |
| 181 | ppvpnpdyep irkgqrdlys glnqrri |

An exemplary human CD3G amino acid sequence is set forth below (SEQ ID NO: 101; GenBank Accession No: NM_000733.3,Version 3, incorporated herein by reference):

| | |
|---|---|
| 1 | tattgtcaga gtcctcttgt ttggccttct aggaaggctg tgggacccag ctttcttcaa |
| 61 | ccagtccagg tggaggcctc tgccttgaac gtttccaagt gaggtaaaac ccgcaggccc |
| 121 | agaggcctct ctacttcctg tgtggggttc agaaaccctc ctcccctccc agcctcaggt |
| 181 | gcctgcttca gaaaatgaag tagtaagtct gctggcctcc gccatcttag taaagtaaca |
| 241 | gtcccatgaa acaaagatgc agtcgggcac tcactggaga gttctgggcc tctgcctctt |
| 301 | atcagttggc gtttgggggc aagatggtaa tgaagaaatg ggtggtatta cacagacacc |
| 361 | atataaagtc tccatctctg gaaccacagt aatattgaca tgccctcagt atcctggatc |
| 421 | tgaaatacta tggcaacaca atgataaaaa cataggcggt gatgaggatg ataaaaacat |
| 481 | aggcagtgat gaggatcacc tgtcactgaa ggaattttca gaattggagc aaagtggtta |
| 541 | ttatgtctgc taccccagag gaagcaaacc agaagatgcg aacttttatc tctacctgag |
| 601 | ggcaagagtg tgtgagaact gcatggagat ggatgtgatg tcggtggcca caattgtcat |
| 661 | agtggacatc tgcatcactg ggggcttgct gctgctggtt tactactgga gcaagaatag |
| 721 | aaaggccaag gccaagcctg tgacacgagg agcgggtgct ggcggcaggc aaaggggaca |
| 781 | aaacaaggag aggccaccac ctgttcccaa cccagactat gagcccatcc ggaaaggcca |
| 841 | gcgggacctg tattctggcc tgaatcagag acgcatctga ccctctggag aacactgcct |
| 901 | cccgctggcc caggtctcct ctccagtccc cctgcgactc cctgtttcct gggctagtct |
| 961 | tggaccccac gagagagaat cgttcctcag cctcatggtg aactcgcgcc ctccagcctg |
| 1021 | atcccccgct ccctcctccc tgccttctct gctggtaccc agtcctaaaa tattgctgct |
| 1081 | tcctcttcct ttgaagcatc atcagtagtc acaccctcac agctggcctg ccctcttgcc |
| 1141 | aggatattta tttgtgctat tcactccctt ccctttggat gtaacttctc cgttcagttc |
| 1201 | cctccttttc ttgcatgtaa gttgtccccc atcccaaagt attccatcta cttttctatc |
| 1261 | gccgtcccct tttgcagccc tctctgggga tggactgggt aaatgttgac agaggccctg |
| 1321 | ccccgttcac agatcctggc cctgagccag ccctgtgctc ctccctcccc caacactccc |
| 1381 | taccaacccc ctaatcccct actccctcca ccccccctcc actgtaggcc actggatggt |
| 1441 | catttgcatc tccgtaaatg tgctctgctc ctcagctgag agagaaaaaa ataaactgta |
| 1501 | tttggctgca agaaaaaaaa aaaaaaaaaa aaaa |

An exemplary human CD3G amino acid sequence is set forth below (SEQ ID NO: 102; GenBank Accession No: P09693.1, Version 1, incorporated herein by reference):

| | |
|---|---|
| 1 | meqgkglavl ilaiillqgt laqsikgnhl vkvydyqedg svlltcdaea knitwfkdgk |
| 61 | migfltedkk kwnlgsnakd prgmyqckgs qnkskplqvy yrmcqnciel naatisgflf |
| 121 | aeivsifvla vgvyfiagqd gvrqsrasdk qtllpndqly qplkdreddq yshlqgnqlr |
| 181 | rn |

An exemplary human CD3G nucleic acid sequence is set forth below (SEQ ID NO: 103; GenBank Accession No: NM_000073.2, Version 2, incorporated herein by reference):

| | |
|---|---|
| 1 | agtctagctg ctgcacaggc tggctggctg gctggctgct aagggctgct ccacgctttt |
| 61 | gccggaggac agagactgac atggaacagg ggaagggcct ggctgtcctc atcctggcta |
| 121 | tcattcttct tcaaggtact ttggcccagt caatcaaagg aaaccacttg gttaaggtgt |
| 181 | atgactatca agaagatggt tcggtacttc tgacttgtga tgcagaagcc aaaaatatca |
| 241 | catggtttaa agatgggaag atgatcggct tcctaactga agataaaaaa aaatggaatc |
| 301 | tgggaagtaa tgccaaggac cctcgaggga tgtatcagtg taaaggatca cagaacaagt |
| 361 | caaaaccact ccaagtgtat tacagaatgt gtcagaactg cattgaacta aatgcagcca |
| 421 | ccatatctgg ctttctcttt gctgaaatcg tcagcatttt cgtccttgct gttggggtct |
| 481 | acttcattgc tggacaggat ggagttcgcc agtcgagagc ttcagacaag cagactctgt |
| 541 | tgcccaatga ccagctctac cagcccctca aggatcgaga agatgaccag tacagccacc |
| 601 | ttcaaggaaa ccagttgagg aggaattgaa ctcaggactc agagtagtcc aggtgttctc |
| 661 | ctcctattca gttcccagaa tcaaagcaat gcattttgga aagctcctag cagagagact |
| 721 | ttcagcccta aatctagact caaggttccc agagatgaca aatggagaag aaaggccatc |
| 781 | agagcaaatt tgggggtttc tcaaataaaa taaaaataaa aacaaatact gtgtttcaga |
| 841 | agcgccacct attggggaaa attgtaaaag aaaaatgaaa agatcaaata accccctgga |
| 901 | tttgaatata attttttgtg ttgtaatttt tatttcgttt ttgtataggt tataattcac |
| 961 | atggctcaaa tattcagtga aagctctccc tccaccgcca tcccctgcta cccagtgacc |
| 1021 | ctgttgccct cttcagagac aaattagttt ctcttttttt tttttttttt tttttttttg |
| 1081 | agacagtctg gctctgtcac ccaggctgaa atgcagtggc accatctcgg ctcactgcaa |
| 1141 | cctctgcctc ctgggttcaa gcgattctcc tgcctcagcc tcccgggcag ctgggattac |
| 1201 | aggcacacac taccacacct ggctaatttt tgtattttta gtagagacag ggttttgctc |
| 1261 | tgttggccaa gctggtctcg aactcctgac ctcaagtgat ccgcccgcct c |

An exemplary human LCK amino acid sequence is set forth below (SEQ ID NO: 104; GenBank Accession No: P06239.6, Version 6, incorporated herein by reference):

| | |
|---|---|
| 1 | mgcgcsshpe ddwmenidvc enchypivpl dgkgtllirn gsevrdplvt yegsnppasp |
| 61 | lqdnlvialh syepshdgdl gfekgeqlri leqsgewwka gslttggegf ipfnfvakan |
| 121 | slepepwffk nlsrkdaerq llapgnthgs fliresesta gsfslsvrdf dqnqgevvkh |
| 181 | ykirnldngg fyispritfp glhelvrhyt nasdglctrl srpcqtqkpq kpwwedewev |
| 241 | pretlklver lgagqfgevw mgyynghtkv avkslkqgsm spdaflaean lmkqlqhqrl |
| 301 | vrlyavvtqe piyiiteyme ngslvdflkt psgikltink lldmaaqiae gmafieerny |
| 361 | ihrdlraani lvsdtlscki adfglarlie dneytarega kfpikwtape ainygtftik |
| 421 | sdvwsfgill teivthgrip ypgmtnpevi qnlergyrmv rpdncpeely qlmrlcwker |
| 481 | pedrptfdyl rsvledffta teggyqpqp |

An exemplary human LCK nucleic acid sequence is set forth below (SEQ ID NO: 105; GenBank Accession No: AH002862.2, Version 2, incorporated herein by reference):

| | |
|---|---|
| 1 | gaattcgaac tgttgcccta ctctccaacc atgattaatg ggtgttgtcc tggcctctga |
| 61 | ctacagcagg ggccgttact atgccctctt gaagacatga ggttgtcctg tctgcctcct |
| 121 | gaaacaggct gttttccagc attctgtctg taagagggat ggtagcctgc cattcaccta |
| 181 | cccttgacta taataaagct actgttccat gccctgagat gacatgggaa ttgttctctc |
| 241 | ggcctgacct gactgtaaca tgcatggtct gctcaccagc tatttaacag ggatattgtc |
| 301 | ctctcctctg actctgatac gatgctacct ttgctgccag acagaaacca aaagggtctc |
| 361 | tcagctgcaa ctggtggtgc tgaggtgctg tttgcctctc accataagct gagtgtgtgt |
| 421 | ccgcttgccc cctgctcact gggcccaaag gctgcccttg aatctcttgc ccagatgcac |
| 481 | cctggagggc agaagggagg gtctatcaga catcctcccc tcaactttaa acctcccagt |
| 541 | gtcaccctgg gacagtaggg gaagatggac ctggtctgga gatgtagggg acccccaggg |
| 601 | gctgagaggc aggggtctat ggtggcagga agcttggcgt gctagagggt tgtggttggg |
| 661 | ctgctggggc ccggttggct gcggagcctc cggaggaggc aggaagtcag ggtgggacgt |
| 721 | gggcgcgggg agacaggtgg tggctacgac ggcgaaggga gctgagactg tccaggcagc |
| 781 | caggttaggc caggaggacc atgtgaatgg ggccagaggg ctcccgggct gggcaggtaa |
| 841 | ggagcgctgg tattgggcgc aggcgccggg gtgagaggcc tgatagcaga cgctgcagnn |
| 901 | nnnnnnnnnn nnnnnnnnnn nnnnnnnnnn nnnnnnnnnn nnnnnnnnnn nnnnnnnnnn |
| 961 | nnnnnnnnnn nnnnnnnnnn nnnnnnnnnn nnnnnnnngg gactccgggg gcttcaaagt |
| 1021 | tgagggcccc acctctgctt cagcgcaaaa caggcacaca tttatcactt tactcctatg |
| 1081 | gagttctgct tgatttcatc agacaaaaaa tttccactcc taaaacacgg caaataaaca |
| 1141 | aaaaaaaagt tatggccaac cagagtcact ggagggtttt ctcctgggga gaagcaagcc |
| 1201 | cgtgtttgaa ggaaccctgt gagatgactg tgggctgtgt gaggggaaca gcgggggctt |
| 1261 | gatggtggac ttcgggagca gaagcctctt tctcagcctc ctcagctaga caggggaatt |
| 1321 | ataataggag gtgtggcgtg cacacctctc cagtagggga gggtctgata agtcaggtct |
| 1381 | ctcccaggct tgggaaagtg tgtgtcatgc tctaggaggt ggtcctccca acacagggta |
| 1441 | ctggcagagg gagagggagg gggcagaggc aggaagtggg taactagact aacaaaggtg |
| 1501 | cctgtggcgg tttgcccatc ccaggtgggg agggtggggc tagggctcag gggccgtgtg |
| 1561 | tgaatttact tgtagcctga gggctcagag ggagcaccgg tttggagctg ggacccccta |
| 1621 | ttttagcttt tctgtggctg gtgaatgggg atcccaggat ctcacaatct caggtacttt |
| 1681 | tggaactttc cagggcaagg ccccattata tctgatgttg ggggagcaga tcttggggga |
| 1741 | gccccttcag ccccctcttc cattccctca gggaccatgg gctgtggctg cagctcacac |
| 1801 | ccggaagatg actggatgga aaacatcgat gtgtgtgaga actgccatta tcccatagtc |
| 1861 | ccactggatg gcaagggcac ggtaagaggc gagacagggg ccttggtgag ggagttgggt |
| 1921 | agagaatgca acccaggaga aagaaatgac cagcacttac aggcccttga aag |

An exemplary human T cell receptor alpha amino acid sequence is set forth below (SEQ ID NO: 106; GenBank Accession No: ALC78508.1, Version 1, incorporated herein by reference):

| | |
|---|---|
| 1 | mkslrvllvi lwlqlswvws qgkeveqnsg plsvpegaia slnctysdrg sgsffwyrqy |
| 61 | sgkspelims iysngdkedg rftaqlnkas qyvsllirds qpsdsatylc avetsgtyky |
| 121 | ifgtgtrlkv laniqnpdpa vyqlrdskss dksvclftdf dsqtnvsgsk dsdvyitdkt |
| 181 | vldmrsmdfk snsavawsnk sdfacanafn nsiipedtff pspesscdvk lveksfetdt |
| 241 | nlnfqnlsvi gfrilllkva gfnllmtlrl wss |

An exemplary human T cell receptor alpha nucleic acid sequence is set forth below (SEQ ID NO: 107; GenBank Accession No: M27377.1, Version 1, incorporated herein by reference):

| | |
|---|---|
| 1 | atggcctctg cacccatctc gatgcttgcg atgctcttca cattgagtgg gctgagagct |
| 61 | cagtcagtgg ctcagcggaa gatcaggtca acgttgctga agggaatcct ctgactgtga |
| 121 | aatgcaccta ttcagtctct ggaaaccctt atcttttttg gtatgttcaa taccccaacc |
| 181 | gaggcctcca gttccttctg aaatacatca caggggataa cctggttaaa ggcagctatg |
| 241 | gctttgaagc tgaatttaac aagagccaaa cctccttcca cctgaagaaa ccatctgccc |
| 301 | ttgtgagcga ctccgctttg tacttctgtg ctgtgagacc cgacagaggc tcaaccctgg |
| 361 | ggaggctata ctttggaaga ggaactcagt tgactgtctg gcctgatatc cagaaccct |

An exemplary human T cell receptor beta amino acid sequence is set forth below (SEQ ID NO: 108; GenBank Accession No: CAA39990.1, Version 1, incorporated herein by reference):

| | |
|---|---|
| 1 | vafcllveel ieagvvqspr ykiiekkqpv afwcnpisgh ntlywylqnl gqgpelliry |
| 61 | eneeavddsq Lpkdrfsaer lkgvdstlki qpaelgdsav ylcassstgf ntgelffgeg |
| 121 | srltvledlk nvfppevavf |

An exemplary human T cell receptor beta nucleic acid sequence is set forth below (SEQ ID NO: 109; GenBank Accession No: L06888.1, Version 1, incorporated herein by reference):

| | |
|---|---|
| 1 | atgggctgca ggctgctctg ctgtgcggtt ctctgtctcc tgggagcggt ccccatggaa |
| 61 | acgggagtta cgcagacacc aagacacctg gtcatgggaa tgacaaataa gaagtctttg |
| 121 | aaatgtgaac aacatctggg tcataacgct atgtattggt acaagcaaag tgctaagaag |
| 181 | ccactggagc tcatgtttgt ctacagtctt gaagaacgtg ttgaaaacaa cagtgtgcca |
| 241 | agtcgcttct cacctgaatg ccccaacagc tctcacttat tccttcacct acacaccctg |
| 301 | cagccagaag actcggccct gtatctctgc gccagcagc |

An exemplary human GNLY amino acid sequence is set forth below (SEQ ID NO: 110; GenBank Accession No: CAG46657.1, Version 1, incorporated herein by reference):

| | |
|---|---|
| 1 | matwalllla amllgnpglv fsrlspeyyd larahlrdee kscpclaqeg pqgdlltktq |
| 61 | elgrdyrtcl tivqklkkmv dkptqrsysn aatrvcrtgr srwrdvcrnf mrryqsrvtq |
| 121 | glvagetaqq icedlrlcip stgpl |

An exemplary human GNLY nucleic acid sequence is set forth below (SEQ ID NO: 111; GenBank Accession No: NM_001302758.1, Version 1, incorporated herein by reference):

| | |
|---|---|
| 1 | gtatctgtgg taaacccagt gacacggggg agatgacata caaaaagggc aggacctgag |
| 61 | aaagattaag ctgcaggctc cctgcccata aaacagggtg tgaaaggcat ctcagcggct |
| 121 | gccccaccat ggctacctgg gccctcctgc tccttgcagc catgctcctg ggcaacccag |
| 181 | gccttgaggt cagtgtgagc cccaagggca agaacacttc tggaagggag agtggatttg |
| 241 | gctgggccat ctggatggaa ggtctggtct tctctcgtct gagccctgag tactacgacc |
| 301 | tggcaagagc ccacctgcgt gatgaggaga aatcctgccc gtgcctggcc caggagggcc |
| 361 | cccagggtga cctgttgacc aaaacacagg agctgggccg tgactacagg acctgtctga |
| 421 | cgatagtcca aaaactgaag aagatggtgg ataagcccac ccagagaagt gtttccaatg |
| 481 | ctgcgacccg ggtgtgtagg acggggaggt cacgatggcg cgacgtctgc agaaatttca |
| 541 | tgaggaggta tcagtctaga gttacccagg gcctcgtggc cggagaaact gcccagcaga |
| 601 | tctgtgagga cctcaggttg tgtatacctt ctacaggtcc cctctgagcc ctctcacctt |
| 661 | gtcctgtgga agaagcacag gctcctgtcc tcagatcccg ggaacctcag caacctctgc |
| 721 | cggctcctcg cttcctcgat ccagaatcca ctctccagtc tccctcccct gactccctct |
| 781 | gctgtcctcc cctctcacga gaataaagtg tcaagcaaga ttttagccgc agctgcttct |
| 841 | tctttggtgg atttgagggg tgggtgtcag tggcatgctg gggtgagctg tgtagtcctt |
| 901 | caataaatgt ctgtcgtgtg tccc |

An exemplary human GZMA amino acid sequence is set forth below (SEQ ID NO: 112; GenBank Accession No: CAG33249.1, Version 1, incorporated herein by reference):

| | |
|---|---|
| 1 | mrnsyrflas slsvvvslll ipedvcekii ggnevtphsr pymvllsldr kticagalia |
| 61 | kdwvltaahc nlnkrsqvil gahsitreep tkqimlvkke fpypcydpat regdlkllql |
| 121 | tekakinkyv tilhlpkkgd dvkpgtmcqv agwgrthnsa swsdtlrevn itiidrkvcn |
| 181 | drnhynfnpv igmnmvcags lrggrdscng dsgspllceg vfrgvtsfgl enkcgdprgp |
| 241 | gvyillskkh lnwiimtikg av |

An exemplary human GZMA nucleic acid sequence is set forth below (SEQ ID NO: 113; GenBank Accession No: NM_006144.3, Version 3, incorporated herein by reference):

| | |
|---|---|
| 1 | agattttcag gttgattgat gtgggacagc agccacaatg aggaactcct atagatttct |
| 61 | ggcatcctct ctctcagttg tcgtttctct cctgctaatt cctgaagatg tctgtgaaaa |
| 121 | aattattgga ggaaatgaag taactcctca ttcaagaccc tacatggtcc tacttagtct |
| 181 | tgacagaaaa accatctgtg ctggggcttt gattgcaaaa gactgggtgt tgactgcagc |
| 241 | tcactgtaac ttgaacaaaa ggtcccaggt cattcttggg gctcactcaa taaccaggga |
| 301 | agagccaaca aaacagataa tgcttgttaa gaaagagttt ccctatccat gctatgaccc |
| 361 | agccacacgc gaaggtgacc ttaaactttt acagctgacg gaaaaagcaa aaattaacaa |
| 421 | atatgtgact atccttcatc tacctaaaaa gggggatgat gtgaaaccag gaaccatgtg |
| 481 | ccaagttgca gggtggggca ggactcacaa tagtgcatct tggtccgata ctctgagaga |
| 541 | agtcaatatc accatcatag acagaaaagt ctgcaatgat cgaaatcact ataattttaa |
| 601 | ccctgtgatt ggaatgaata tggtttgtgc tggaagcctc cgaggtggaa gagactcgtg |
| 661 | caatggagat tctggaagcc ctttgttgtg cgagggtgtt ttccgagggg tcacttcctt |
| 721 | tggccttgaa aataaatgcg gagaccctcg tgggcctggt gtctatattc ttctctcaaa |
| 781 | gaaacacctc aactggataa ttatgactat caagggagca gtttaaataa ccgtttcctt |
| 841 | tcatttactg tggcttctta atcttttcac aaataaaatc aatttgcatg actgtaaaaa |
| 901 | aaaaaaaaaa aaa |

An exemplary human GZMB amino acid sequence is set forth below (SEQ ID NO: 114; GenBank Accession No: P10144.2, Version 2, incorporated herein by reference):

| | |
|---|---|
| 1 | mqpillllaf lllpradage iiggheakph srpymaylmi wdqkslkrcg gflirddfvl |
| 61 | taahcwgssi nvtlgahnik eqeptqqfip vkrpiphpay npknfsndim llqlerkakr |
| 121 | travqplrlp snkaqvkpgq tcsvagwgqt aplgkhshtl qevkmtvqed rkcesdlrhy |
| 181 | ydstielcvg dpeikktsfk gdsggplvcn kvaqgivsyg rnngmpprac tkvssfvhwi |
| 241 | kktmkry |

An exemplary human GZMB nucleic acid sequence is set forth below (SEQ ID NO: 115; GenBank Accession No: NM_004131.4, Version 4, incorporated herein by reference):

| | |
|---|---|
| 1 | ccaagagcta aaagagagca aggaggaaac aacagcagct ccaaccaggg cagccttcct |
| 61 | gagaagatgc aaccaatcct gcttctgctg gccttcctcc tgctgcccag ggcagatgca |
| 121 | ggggagatca tcgggggaca tgaggccaag ccccactccc gcccctacat ggcttatctt |
| 181 | atgatctggg atcagaagtc tctgaagagg tgcggtggct tcctgatacg agacgacttc |
| 241 | gtgctgacag ctgctcactg ttggggaagc tccataaatg tcaccttggg ggcccacaat |
| 301 | atcaaagaac aggagccgac ccagcagttt atccctgtga aaagacccat cccccatcca |
| 361 | gcctataatc ctaagaactt ctccaacgac atcatgctac tgcagctgga gagaaaggcc |
| 421 | aagcggacca gagctgtgca gcccctcagg ctacctagca acaaggccca ggtgaagcca |
| 481 | gggcagacat gcagtgtggc cggctggggg cagacggccc ccctgggaaa acactcacac |
| 541 | acactacaag aggtgaagat gacagtgcag gaagatcgaa agtgcgaatc tgacttacgc |
| 601 | cattattacg acagtaccat tgagttgtgc gtgggggacc cagagattaa aaagacttcc |
| 661 | tttaaggggg actctggagg ccctcttgtg tgtaacaagg tggcccaggg cattgtctcc |
| 721 | tatggacgaa acaatggcat gcctccacga gcctgcacca aagtctcaag ctttgtacac |
| 781 | tggataaaga aaaccatgaa acgctactaa ctacaggaag caaactaagc ccccgctgta |
| 841 | atgaaacacc ttctctggag ccaagtccag atttacactg ggagaggtgc cagcaactga |
| 901 | ataaatacct cttagctgag tggaaaaaaa aaaaaaaaaa a |

An exemplary human GZMH amino acid sequence is set forth below (SEQ ID NO: 116; GenBank Accession No: P20718.1, Version 1, incorporated herein by reference):

| | |
|---|---|
| 1 | mqpfllllaf lltpgagtee iiggheakph srpymafvqf lqeksrkrcg gilvrkdfvl |
| 61 | taahcqgssi nvtlgahnik eqertqqfip vkrpiphpay npknfsndim llqlerkakw |
| 121 | ttavrplrlp sskaqvkpgq lcsvagwgyv smstlattlq evlltvqkdc qcerlfhgny |
| 181 | srateicvgd pkktqtgfkg dsggplvckd vaqgilsygn kkgtppgvyi kvshflpwik |
| 241 | rtmkrl |

An exemplary human GZMH nucleic acid sequence is set forth below (SEQ ID NO: 117; GenBank Accession No: NM_033423.4, Version 4, incorporated herein by reference):

| | |
|---|---|
| 1 | gaggtctctg agtttactgt acccatccct ccttcatctc cctccagcat ttgtttctgg |
| 61 | aaggagtcaa caccaacagc tctgacctgg gcagccttcc tgagaaaatg cagccattcc |
| 121 | tcctcctgtt ggcctttctt ctgacccctg gggctgggac agaggagatc atcgggggcc |
| 181 | atgaggccaa gccccactcc cgcccctaca tggcctttgt tcagtttctg caagagaaga |
| 241 | gtcggaagag gtgtggcggc atcctagtga gaaaggactt tgtgctgaca gctgctcact |
| 301 | gccagggaag ctccataaat gtcaccttgg gggcccacaa tatcaaggaa caggagcgga |
| 361 | cccagcagtt tatccctgtg aaaagaccca tcccccatcc agcctataat cctaagaact |
| 421 | tctccaacga catcatgcta ctgcagctgg agagaaaggc caagtggacc acagctgtgc |
| 481 | ggcctctcag gctacctagc agcaaggccc aggtgaagcc agggcagctg tgcagtgtgg |
| 541 | ctggctgggg ttatgtctca atgagcactt tagcaaccac actgcaggaa gtgttgctga |
| 601 | cagtgcagaa ggactgccag tgtgaacgtc tcttccatgg caattacagc agagccactg |
| 661 | agatttgtgt gggggatcca aagaagacac agaccggttt caagggggac tccggggggc |
| 721 | ccctcgtgtg taaggacgta gcccaaggta ttctctccta tggaaacaaa aaagggacac |
| 781 | ctccaggagt ctacatcaag gtctcacact tcctgccctg gataaagaga acaatgaagc |
| 841 | gcctctaaca gcaggcatga gactaacctt cctctgggcc tgaccatctc tgggacagag |
| 901 | gcaagaatcc ccaaggggtg ggcagtcggg gttgcaggac tgtaataaat ggatctctgg |
| 961 | tgtaaatatg aaaaaaaaaa aaaaaaa |

An exemplary human GZMK amino acid sequence is set forth below (SEQ ID NO: 118; GenBank Accession No: P49863.1, Version 1, incorporated herein by reference):

| | |
|---|---|
| 1 | mtkfssfslf flivgaymth vcfnmeiigg kevsphsrpf masiqygghh vcggvlidpq |
| 61 | wvltaahcqy rftkgqsptv vlgahslskn easkqtleik kfipfsrvts dpqsndimlv |
| 121 | klqtaaklnk hvkmlhirsk tslrsgtkck vtgwgatdpd slrpsdtlre vtvtvlsrkl |
| 181 | cnsqsyyngd pfitkdmvca gdakgqkdsc kgdsggplic kgvfhaivsg ghecgvatkp |
| 241 | giytlltkky qtwiksnlvp phtn |

An exemplary human GZMK nucleic acid sequence is set forth below (SEQ ID NO: 119; GenBank Accession No: NM_002104.2, Version 2, incorporated herein by reference):

| | |
|---|---|
| 1 | gatcaacaca tttcatctgg gcttcttaaa tctaaatctt taaaatgact aagttttctt |
| 61 | ccttttctct gtttttccta atagttgggg cttatatgac tcatgtgtgt ttcaatatgg |
| 121 | aaattattgg agggaaagaa gtgtcacctc attccaggcc atttatggcc tccatccagt |
| 181 | atggcggaca tcacgtttgt ggaggtgttc tgattgatcc acagtgggtg ctgacagcag |
| 241 | cccactgcca atatcggttt accaaaggcc agtctcccac tgtggtttta ggcgcacact |
| 301 | ctctctcaaa gaatgaggcc tccaaacaaa cactggagat caaaaaattt ataccattct |
| 361 | caagagttac atcagatcct caatcaaatg atatcatgct ggttaagctt caaacagccg |
| 421 | caaaactcaa taaacatgtc aagatgctcc acataagatc caaaacctct cttagatctg |
| 481 | gaaccaaatg caaggttact ggctggggag ccaccgatcc agattcatta agaccttctg |
| 541 | acaccctgcg agaagtcact gttactgtcc taagtcgaaa actttgcaac agccaaagtt |
| 601 | actacaacgg cgaccctttt atcaccaaag acatggtctg tgcaggagat gccaaaggcc |
| 661 | agaaggattc ctgtaagggt gactcagggg gccccttgat ctgtaaaggt gtcttccacg |
| 721 | ctatagtctc tggaggtcat gaatgtggtg ttgccacaaa gcctggaatc tacaccctgt |
| 781 | taaccaagaa ataccagact tggatcaaaa gcaaccttgt cccgcctcat acaaattaag |
| 841 | ttacaaataa ttttattgga tgcacttgct tcttttttcc taatatgctc gcaggttaga |
| 901 | gttgggtgta agtaaagcag agcacatatg gggtccattt ttgcacttgt aagtcatttt |
| 961 | attaaggaat caagttcttt ttcacttgta tcactgatgt atttctacca tgctggtttt |
| 1021 | attctaaata aaatttagaa gactcaaaaa aaaaaaaaaa aaaaaaaaaa aaaa |

An exemplary human PRF1 amino acid sequence is set forth below (SEQ ID NO: 120; GenBank Accession No: P14222.1, Version 1, incorporated herein by reference):

| | |
|---|---|
| 1 | maarllllgi lllllplpvp apchtaarse ckrshkfvpg awlagegvdv tslrrsgsfp |
| 61 | vdtqrflrpd gtctlcenal qegtlqrlpl altnwraqgs gcqrhvtrak vssteavard |
| 121 | aarsirndwk vgldvtpkpt snvhvsvags hsqaanfaaq kthqdqysfs tdtvecrfys |
| 181 | fhvvhtpplh pdfkralgdl phhfnastqp aylrlisnyg thfiravelg grisaltalr |
| 241 | tcelaleglt dnevedcltv eaqvnigihg sisaeakace ekkkkhkmta sfhqtyrerh |
| 301 | sevvgghhts indllfgiqa gpeqysawvn slpgspglvd ytleplhvll dsqdprreal |
| 361 | rralsqyltd rarwrdcsrp cppgrqkspr dpcqcvchgs avttqdccpr qrglaqlevt |
| 421 | fiqawglwgd wftatdayvk lffggqelrt stvwdnnnpi wsvrldfgdv llatggplrl |
| 481 | qvwdqdsgrd ddllgtcdqa pksgshevrc nlnhghlkfr yharclphlg ggtcldyvpq |
| 541 | mllgeppgnr sgavw |

An exemplary human PRF1 nucleic acid sequence is set forth below (SEQ ID NO: 121; GenBank Accession No: M31951.1, Version 1, incorporated herein by reference):

| | |
|---|---|
| 1 | gaattccaaa gtcctctctt tgattttata ggtgaggaaa ctaacgctca gaaagggggt |
| 61 | tgatatctat cgccgtgagg catacggtaa gtttctggtg aagctgggat cagaacctgt |
| 121 | ttagactttg cctctctttt cccgcagata ctttgcagga cttctatgtc cctcaaaccg |
| 181 | gccttcctgt catggtcagg aaagaaactc ctcacagcct cagcatccaa gtcaggccat |
| 241 | gggtgacagc tggaaagtga tcaggaggct gcagtttcta gaagagggtg gggacactgc |
| 301 | ggagagaaga tggggccaga ttccgagaag acagcataag cccctgttcc tgtaagagca |
| 361 | gggacggaag cagggacata aacgcaaggg atgagcccca aagtgtgacc catgagacat |
| 421 | gatgtcacat gtggtctgga gcctgcccca cttcttccca tcatatacac agttatgaga |
| 481 | acaagttgtg agaaccacct cctcccttac ccagctgccc ccaccccaga agccgtgtga |
| 541 | ttttgccccc cagtgccctg tgagtcactc cacccatgga aacctcaccc caccctgacc |
| 601 | tcaagcaagg cagagtgcag aagacatgtc ctccggtgct accagaccac tctcaccagc |
| 661 | acccacgacc tcagcagggc tggagccagc gtggaggcca ctggctgtcc tcacaaagcg |
| 721 | aggagcagga gcccctgttc gaggaacatg cttggagttc ggagcctggg ctagggtggg |
| 781 | atgtaggttg agcaggaagt ggatggcaag attagagcaa catctctctt ctcccactca |
| 841 | gggaggaggg aatggccaca ggctctgaca ctcaagaagg gccaggcaca gttccaagca |
| 901 | cttcacaaca acccctaggg tctacatgac ctacaatccc aattgttcag tgaagaaact |
| 961 | gaggcacagt gaggctgaag aaccctacca gtccacactg ctggtgcata accgagctgc |
| 1021 | ccaagccccg gcggtctggc gtgtaggccc atgctctgag ccgccgcctc tgcttgcctc |
| 1081 | ttacatccca cacatgcgat gctgtgcatc agaagcaagg agatggccct gctggcctgt |
| 1141 | tcatcaacac cagggccgag tctcaaagtc ctcagcgccc cgccctcctc cgcctgtgtg |
| 1201 | ccctgagtcc ccgagcccca gcagctctac tcggcagatg agcctctggc cctgctgctc |
| 1261 | gcttcctgag ggctgtcagt ggggagccgg atgagggctg aggacagggt gggtgctcgt |
| 1321 | gggaggggag agcacaaagg acctgtgacc acagctgggg gcggggcagg aagtagaagt |
| 1381 | gatgtgagtg gtggctggtg caaggagcca cagtgggctg cctggggggc tgatgccacc |
| 1441 | attccaggag cctcggtgaa gagaggatat ccatctgtgt agccgcttct ctatacggga |
| 1501 | ttccaggtaa ggagagagca gggattgggg gcctggggcc ctgggtggag gggaagaggc |
| 1561 | tgatggagca ggaagtgctg tgacctataa gacaagacac ctgggtcaca gacggtgccc |
| 1621 | atcactaact cgctgggcag ccctgtgtcc accctgggcc tcagtttcct catgtatgaa |
| 1681 | atgaagaggt agcgtgcagt ttctaaggcc ctgcaagtgc tgacattaaa gcttctaaga |
| 1741 | aagctggaaa gaggctccct gggacagaat accatgagag tcaggatgag ggctgagttc |
| 1801 | actggattag ggttatgact gtgcccgcct cctagctggg atgagcccag ggcctttgaa |
| 1861 | gattcccctg gcctctttgc ctccctgggg cgagccacgc tgcctgaaat tccacccttc |
| 1921 | aagtcacacc tttgggcagg gcaggaggct ccagctataa tgggggctct ccatagccct |
| 1981 | cttgtctcag aacctgtggt acccaagggc aaaggccttt gaagagctca gcttggataa |
| 2041 | ggttaggctt gggtaaggtt agagaaagga ggttccagat cataaacagg cccaggcaag |
| 2101 | gccccagaaa caccttaaaa ttaggaagaa ccttccagac aatcattgtc catagtaggc |
| 2161 | aatagggggg cccctgtcac tgccgaaggc cacagagctt ggcagggagt ggcaaagagg |
| 2221 | ggaggggcag caagaaagga tctgaagagc aaccatcgag gcctctcccc acacaaaatc |
| 2281 | caagggtgtg gtcaggtctg cccctttttc ttttttttct ttctcttttt tttttttttt |
| 2341 | tttttttgag gcagagtctt actctatcac ccaggcggga gtgcagtggt acgatcttgg |
| 2401 | ctcactgcaa cctccgcctc ctgggttcaa gcgattctcc tgtctcagcc tcccaagtag |
| 2461 | ctgggattac aggcacccgc caccatgccc agctaatttt tgtattttta gtaaagaggg |
| 2521 | ggtttcacca cgttggccag gctggtctcg aacttctgac ctccagtgat ccacctgcct |
| 2581 | ctgcctccca cagtgttcgg tttacaggtg tgagccacca cacctggcca ggtcggcaga |
| 2641 | ttttattcta gaattcgggc tgatgttatt ccataaagct cagagggaga gcgccaaagg |
| 2701 | gaaaatgaga agaataggtg caatatagga cctcatttct cttccaattc tgttaagggc |
| 2761 | tcagtgggag ggagagaggt tagagagagt gacagagaca aagaaacaga gtgagagtca |
| 2821 | gggtgggcaa agggtggctt gttctggggc caaaaatgac acttcttcag aatgaaggct |
| 2881 | tcctcaggtc cccagacccc tccctaaacc tgctacagct actctgtccc ctcttctgtg |
| 2941 | gccacagccc cctccccacc acccacagtt gtgtcctggg gacagagcca tccacctaga |
| 3001 | tccccattag gccttaggga aattctaaaa aggggctccc cttggctggg cttttcccct |
| 3061 | ctctgggccc atctgtacaa tgtgggggct gaaccaggcc tataagggac ataccagctc |
| 3121 | tgacattcat tgaataatga cttaagagat atctcagccc ctccccttcc atgtgccctg |
| 3181 | ataatctgtg gctgtggggg aagggagcag tcatcctcca tccctccacc catggcttcc |
| 3241 | cagagcccaa gtgccccctg tctctgcagc tccatggcag cccgtctgct cctcctgggc |
| 3301 | atccttctcc tgctgctgcc cctgcccgtc cctgccccgt gccacacagc cgcacgctca |
| 3361 | gagtgcaagc gcagccacaa gttcgtgcct ggtgcatggc tggccgggga gggtgtggac |
| 3421 | gtgaccagcc tccgccgctc gggctccttc ccagtggaca cacaaaggtt cctgcggccc |
| 3481 | gacggcacct gcaccctctg tgaaaatgcc ctacaggagg gcaccctcca gcgcctgcct |
| 3541 | ctggcgctca ccaactggcg ggcccagggc tctggctgcc agcgccatgt aaccagggcc |
| 3601 | aaagtcagct ccactgaagc tgtggcccgg gatgcggctc gtagcatccg caacgactgg |
| 3661 | aaggtcgggc tggacgtgac tcctaagccc accagcaatg tgcatgtgtc tgtggccggc |
| 3721 | tcacactcac aggcagccaa ctttgcagcc cagaagaccc accaggacca gtacagcttc |
| 3781 | agcactgaca cggtggagtg ccgcttctac aggtgagagc tggggctagg ggtggggggc |
| 3841 | tggaaaaggc gcgggaaact ctgggtggtc taagagccct ggaaaggcag agttctccaa |
| 3901 | tcaaacttgg aggctgcttc caccaggaag aaactgcaat cctggctctc agacttcagg |
| 3961 | tgattcagcc ctgtggcctc ctctcatcca ggaggtccca aatctgggag tgcccagtga |
| 4021 | gacatcaagt agagagaaag tgagtgagac catgcctgag agtcccagca gggtgccatt |
| 4081 | taggtctagt gagtactgaa ccctgccttt cctgggcaca gccagccttc cacagagtcc |
| 4141 | ttcctggata gggacagagg caggagacct ggccgggtca tccttcctca tcgctgtgtg |
| 4201 | accttgagca tctcacttct ctctgagctt caatcatcca ctcaaccaat actcacacct |
| 4261 | gcctgtggca gggccacact gctgagcgac agcagtcact accctagccc ctgggagtag |
| 4321 | gaagtatggg acccatattc cacgtctgag aaaggcacag tgtttgtagg ccagacccaa |
| 4381 | gtcacctggt ctgattcatc agacctgggg gccacgtttc ctccacagag aggccgagca |
| 4441 | tggcccaagc tgtcaggatc ccgggcatgt gggacccatc cagggggacg gatggatgaa |
| 4501 | ggccacatga ctagttccaa agttcgacag ataccatcag tgcaggatca ttgcttttat |
| 4561 | gttctttttt actttttctt aaaaaaaaaa tagagatggg gtctcactat gttgcccagg |
| 4621 | ctggtctcaa actcctgggc tcaagtgatc ctcccgcctc ggcctcccaa agtgctgggg |
| 4681 | ttacaggcat gaaccactgc tcccggccag gatcattgct tttataataa gaaaattaaa |
| 4741 | aggaaagaaa aaaatgttat tttgaaaagg aaaagagaaa atacctatac agagcactga |
| 4801 | ggtccctgag gggtgagagc ggaggcattc ctgccagccc cgtgccactg tgcttgtgct |
| 4861 | ctggagccgg gcccctgggt tccagtccta gttctgccca cttacatgtg accttgagca |
| 4921 | gtcctgaagg agttatttga ttgaatgggg gaaatactcc cctgggccca gctgaggtct |
| 4981 | ctctcttctc gcagtttcca tgtggtacac actcccccgc tgcaccctga cttcaagagg |
| 5041 | gccctcgggg acctgcccca ccacttcaac gcctccaccc agcccgccta cctcaggctt |
| 5101 | atctccaact acggcaccca cttcatccgg gctgtggagc tgggtggccg catatcggcc |
| 5161 | ctcactgccc tgcgcacctg cgagctggcc ctggaagggc tcacggacaa cgaggtggag |
| 5221 | gactgcctga ctgtcgaggc ccaggtcaac ataggcatcc acggcagcat ctctgccgaa |
| 5281 | gccaaggcct gtgaggagaa gaagaagaag cacaagatga cggcctcctt ccaccaaacc |
| 5341 | taccgggagc gccactcgga agtggttggc ggccatcaca cctccattaa cgacctgctg |
| 5401 | ttcgggatcc aggccgggcc cgagcagtac tcagcctggg taaactcgct gcccggcagc |
| 5461 | cctggcctgg tggactacac cctggaaccc ctgcacgtgc tgctggacag ccaggacccg |
| 5521 | cggcgggagg cactgaggag ggccctgagt cagtacctga cggacagggc tcgctggagg |
| 5581 | gactgcagcc ggccgtgccc accagggcgg cagaagagcc cccgagaccc atgccagtgt |
| 5641 | gtgtgccatg gctcagcggt caccacccag gactgctgcc ctcggcagag gggcctggcc |
| 5701 | cagctggagg tgaccttcat ccaagcatgg ggcctgtggg gggactggtt cactgccacg |
| 5761 | gatgcctatg tgaagctctt ctttggtggc caggagctga ggacgagcac cgtgtgggac |
| 5821 | aataacaacc ccatctggtc agtgcggctg gattttgggg atgtgctcct ggccacaggg |
| 5881 | gggcccctga ggttgcaggt ctgggatcag gactctggca gggacgatga cctccttggc |
| 5941 | acctgtgatc aggctcccaa gtctggttcc catgaggtga gatgcaacct gaatcatggc |
| 6001 | cacctaaaat tccgctatca tgccaggtgc ttgccccacc tgggaggagg cacctgcctg |
| 6061 | gactatgtcc cccaaatgct tctgggggag cctccaggaa accggagtgg ggccgtgtgg |
| 6121 | tgagaacagt gagcttggaa aggaccagta tgcttggact gaaggggttc tcacagtggg |
| 6181 | agccagggct gtcttcgtat tcccattaga ccaagctt |

An exemplary human CD19 amino acid sequence is set forth below (SEQ ID NO: 122; GenBank Accession No: AAB60697.1, Version 1, incorporated herein by reference):

| | |
|---|---|
| 1 | mppprllffl lfltpmevrp eeplvvkveg egdnavlqcl kgtsdgptqq ltwsresplk |
| 61 | pflklslglp glgihmrpla swlfifnvsq qmggfylcqp gppsekawqp gwtvnvegsg |
| 121 | elfrwnvsdl gglgcglknr ssegpsspsg klmspklyvw akdrpeiweg eppcvpprds |
| 181 | lnqslsqdlt mapgstlwls cgvppdsvsr gplswthvhp kgpksllsle lkddrpardm |
| 241 | wvmetglllp rataqdagky ychrgnltms fhleitarpv lwhwllrtgg wkvsavtlay |
| 301 | lifclcslvg ilhlgralvl rrkrkrmtdp trrffkvtpp pgsgpqnqyg nvlslptpts |
| 361 | glgraqrwaa glggtapsyg npssdvqadg algsrsppgv gpeeeegegy eepdseedse |
| 421 | fyendsnlgq dqlsqdgsgy enpedeplgp ededsfsnae syenedeelt qpvartmdfl |
| 481 | sphgsawdps reatslgsqs yedmrgilya apqlrsirgq pgpnheedad syenmdnpdg |
| 541 | pdpawggggr mgtwstr |

An exemplary human CD19 nucleic acid sequence is set forth below (SEQ ID NO: 123; GenBank Accession No: M84371.1, Version 1, incorporated herein by reference):

| | |
|---|---|
| 1 | ggatcctctc gcctcggcct cctaaagtat tgggattaca |
| | ggcatgagcc tctgtgcctg |
| 61 | gctgtaactg acatgtttta agcaggggaa tgacatgctc |
| | tagtgaaagc cagtctgggc |
| 121 | agctgggtag ctaatgaggg gattagagag attttgttga |
| | atgaaaggca gattgagtcc |
| 181 | tgctactcgc ccccttcatt ccccttcatt catgcctcat |
| | tcttccgcct cccagccgcc |
| 241 | tcaactggcc aaagggaagt ggaggccctg ccacctgtag |
| | ggagggtccc ctggggcttg |
| 301 | cccacagcaa acaggaagtc acagcctggt gagatgggcc |
| | tgggaatcag ccactgagaa |
| 361 | agtgggtctc ttgggtccct gaattctttt tctgagtccc |
| | tgcagcagtg aaaaagacac |
| 421 | agaggcacat agagagtgac agagaaagag agagacagag |
| | aggagaggca tggggcagaa |
| 481 | taagaacaga tttaggagtt agaactcctg ggttctttta |
| | aaacaatttt tcttttagag |
| 541 | acagggtctt gttgtgttgc ccggactgga gcacagtggc |
| | tattcccagg cataatcatg |
| 601 | gtgcactgca gccttgaact cctgggctca agcgatcctt |
| | ctacctcagc ctcccaagga |
| 661 | cctgggacca taggcgtgta ccactgtgcc tggcttttgc |
| | ctggttttaa actgaggcag |
| 721 | tatgacttga gctcttaggc attaattgaa gctgtatctc |
| | attaactgag ggcttatgat |
| 781 | gtgctggaca ctgggctaat agtgctgaac atattgtcat |
| | ttttaatctt cacaaacaat |
| 841 | atttgtatag gactgttttc ttttcttttt tttttttgaa |
| | acagagtctc actctggtgc |
| 901 | ccaggctgga gtgcagtggt gtgatctcgg ctcactgcaa |
| | cctccgcctc ctggtttcca |
| 961 | gtgattctcc tgcctcagcc tcctaagtag ctgggattac |
| | aggtgtgcgc caccatgccc |
| 1021 | ggctaatttt tttttttttt tttgagaagg agtctatgtg |
| | cccagcattg ttctagagca |
| 1081 | cttgcaatta gtggtgaaca acacggtctc tactccaagg |
| | ggctcacatt cttgtgcaga |
| 1141 | aaacagaaat gaacaaataa acacacaaga tcatttcccg |
| | tggtagtgag agctgggatg |
| 1201 | aaaataaaac agcgtggcag ggaggaggca agtgttgtga |
| | gtctggaggg ttcctggaga |
| 1261 | atggggcctg aggcgtgacc accgccttcc tctctggggg |
| | gactgcctgc cgcccccgca |
| 1321 | gacacccatg gttgagtgcc ctccaggccc ctgcctgccc |
| | cagcatcccc tgcgcgaagc |
| 1381 | tgggtgcccc ggagagtctg accaccatgc cacctcctcg |
| | cctcctcttc ttcctcctct |
| 1441 | tcctcacccc catggaagtc aggcccgagg aacctctagt |
| | ggtgaaggtg gaaggtatgt |
| 1501 | ccaaagggca gaaagggaag ggattgaggc tggaaacttg |
| | agttgtggct gggtgtcctt |
| 1561 | ggctgagtaa cttaccctct ctgagcctcc attttcttat |
| | ttgtaaaatt caggaaaggg |
| 1621 | ttggaaggac tctgccggct cctccactcc cagcttttgg |
| | agtcctctgc tctataacct |
| 1681 | ggtgtgagga gtcggggggc ttggaggtcc cccccaccca |
| | tgcccacacc tctctccctc |
| 1741 | tctctccaca gagggagata acgctgtgct gcagtgcctc |
| | aaggggacct cagatggccc |
| 1801 | cactcagcag ctgacctggt ctcgggagtc cccgcttaaa |
| | cccttcttaa aactcagcct |
| 1861 | ggggctgcca ggcctgggaa tccacatgag gcccctggca |
| | tcctggcttt tcatcttcaa |
| 1921 | cgtctctcaa cagatggggg gcttctacct gtgccagccg |
| | gggcccccct ctgagaaggc |
| 1981 | ctggcagcct ggctggacag tcaatgtgga gggcagcggt |
| | gagggccggg ctggggcagg |
| 2041 | ggcaggagga gagaagggag gccaccatgg acagaagagg |
| | tccgcggcca caatggagct |
| 2101 | ggagagaggg gctggaggga ttgagggcga aactcggagc |
| | taggtgggca gactcctggg |
| 2161 | gcttcgtggc ttcagtatga gctgcttcct gtccctctac |
| | ctctcactgt cttctctctc |
| 2221 | tctgcgggtc tttgtctcta tttatctctg tctttgagtc |
| | tctatctctc tccctctcct |
| 2281 | gggtgtctct gcatttggtt ctgggtctct tcccagggga |
| | gctgttccgg tggaatgttt |
| 2341 | cggacctagg tggcctgggc tgtggcctga agaacaggtc |
| | ctcagagggc cccagctccc |
| 2401 | cttccgggaa gctcatgagc cccaagctgt atgtgtgggc |
| | caaagaccgc cctgagatct |
| 2461 | gggagggaga gcctccgtgt gtcccaccga gggacagcct |
| | gaaccagagc ctcagccagg |
| 2521 | gtatggtgat gactggggag atgccgggaa gcgggggtcc |
| | agagacagag gggaggggaa |
| 2581 | actgaagagg tgaaaccctg aggatcaggc tttccttgtc |
| | ttatctctcc ctgtcccaga |
| 2641 | cctcaccatg gcccctggct ccacactctg gctgtcctgt |
| | ggggtacccc ctgactctgt |
| 2701 | gtccaggggc cccctctcct ggacccatgt gcaccccaag |
| | gggcctaagt cattgctgag |
| 2761 | cctagagctg aaggacgatc gcccggccag agatatgtgg |
| | gtaatggaga cgggtctgtt |
| 2821 | gttgccccgg gccacagctc aagacgctgg aaagtattat |
| | tgtcaccgtg gcaacctgac |
| 2881 | catgtcattc cacctggaga tcactgctcg gccaggtaga |
| | gtttctctca actgggaggc |
| 2941 | atctgtgtgg gggtactggg aagaagtgga agccagtcaa |
| | tcttagattc ccccaacccg |
| 3001 | agggctactc ccagcctcac cccaaacccc aacttccaca |
| | cagaacactg actccaagtc |
| 3061 | tttctttttt ttgacagagt ctcgctctgt tgcctaggct |
| | ggagtgcagt ggtgccatct |
| 3121 | tgtcttggct cactgcaacc tccgcctccc aggttcaagt |
| | gattcccctg cctcagcctc |
| 3181 | ctgagtagct gggattacag gtgcccacca ccacgcctgg |
| | ctaatttttt tttttttttt |
| 3241 | gagacggagt cttgcactgt cacccaggct ggagtgcagt |
| | ggcacgatct cagctcactg |
| 3301 | caacctccac cttccaggtt caagtgattc tcctgcctca |
| | gcctcccgag tagctgggat |
| 3361 | taaagcctgg ctaatttttt ttgtattttt agtagagatg |
| | gggtttcatt atgttggcca |
| 3421 | ggctggtctc aaactcctga cctcgtgatc cacccgcctc |
| | ggcctcccaa agtgctggga |
| 3481 | ttacagacat gagccacagg gccgggccaa gcctaatttt |
| | gtatttttag tagagatggg |
| 3541 | gtttctccct gttggaccag gctggtcttg aactcctgac |
| | ttcaggtgat ctgcctgcct |
| 3601 | tggcctccca aagtactggg attacaggca taagccaccg |
| | cacctggcct agacttcaag |
| 3661 | tctttcttcc ctcgcttcca agacactact tttctgggtc |
| | ttcacctacc attgcttgcg |
| 3721 | cctgcccacc agcttgggtg gagtcttcct tcctccccaa |
| | ctcctcactc ttggagccct |
| 3781 | gggccctctt cttatccctg tctgcacact ttcctatttg |
| | aacttgactc tcaatggctt |
| 3841 | cttgggtcac catgccttgg tgactctatt ccaggctcca |
| | tactcagcca tctcctgtgc |
| 3901 | catttgatat cccatggaca cctcaggctc aacagataca |
| | aaatcaaact caatgtcttc |
| 3961 | cccaagtata gtcttcttgg tggcccagtg taagcagagg |
| | gcaccaccac ctgctccctc |
| 4021 | gcccaggcta agaacctggg catccttctt tttcctcacc |
| | ccgtccaaca aactggtcac |
| 4081 | agtgttctgc caattctctc tccatgcaat cctatcatgc |
| | tatcctaact gcaattcaca |
| 4141 | aacccaaccc caactttcac tccaaacttg atccaagcaa |
| | tgtgctggat cccaactgta |
| 4201 | accttgcaaa ctcaactctg cccttcactt tgaccgtgac |
| | tatccttaat tgcagcagga |
| 4261 | aactgatcat tatgctcccc tcaatccaca cattgcctct |
| | gagtacagcc atggtttgtc |
| 4321 | cacgatttgc tcaaagacac tgcccatgtc ctgtgccagg |
| | gtctgtgaca atccctgacc |
| 4381 | tcctgggaca tggctcctta gagagaggag agcctttctc |
| | acagcttggg actttgagtc |
| 4441 | tgtgtctttt tttttttctt gagacggagt tttgctgtgg |
| | ttgcccaggc tggagtgcag |
| 4501 | tgatctcggc tcactgaaac ctccgcctcc cgggttcaaa |
| | cgattctcct gcctcagcct |
| 4561 | cccaagtagc tgggattaca ggcacccacc accatgccca |
| | gctaattttt ttgtattttt |
| 4621 | agtagagatg gggtttcacc atgttggcca ggctggtctc |
| | gaactcctga cctcaggtga |
| 4681 | tccacccgcc tttgcctccc aaagtgctgg gattacaggc |
| | gtcaaccacc gcgcccggcc |
| 4741 | gagtctgtgt cttgcctctg tgcctcagac ttgcggttcc |
| | ttgagatctc aggattggga |
| 4801 | cgtaagatgc cagcctgggg tcctcgtctc atagcccctt |
| | ccccctagta ctatggcact |
| 4861 | ggctgctgag gactggtggc tggaaggtct cagctgtgac |
| | tttggcttat ctgatcttct |
| 4921 | gcctgtgttc ccttgtgggc attcttcatc ttcaaagagg |
| | tgagtcatgt ccccagtggg |
| 4981 | tctgtccaaa ccctactcca tcttccccag gataagccgg |
| | ctctggccag tctgacaacc |
| 5041 | atctttcttt cctcccatcc ctcccttcaa gaccccagaa |
| | tcctgttctc cccagtcttc |
| 5101 | ctctagcctc cctcaaactt cccaagcctc ttgcaatttt |
| | tttttttttt ttgagacagg |
| 5161 | gtctcattct gtcaccccag ctggagtgca gtggcacaat |
| | ctgagctcac tgtaacctct |
| 5221 | gcctcccagg cttaagtgat tcttgtgctt cagcctcccg |
| | agtacctggg actacaagtg |
| 5281 | tatgccacca cacccggcca attttttata tttttagtag |
| | agacgaggtt tcaccatgtt |
| 5341 | ggccagactg gtctcgaact cttgacctca aatgatccgc |
| | ccacctcggc ctcccaaagt |
| 5401 | gctgggatta caggcacgag ccaccgcgcc cgtccgcctc |
| | gcaatttgaa ctcctgtctc |
| 5461 | ctttgttgaa ccaagtgacc tccccagcac ctggccccac |
| | aaatcctcac cctgccaagc |
| 5521 | agcccctcct ctgatcacgc cctttaactc ccaccagccc |
| | tggtcctgag gaggaaaaga |
| 5581 | aagcgaatga ctgaccccac caggaggtaa tgcaaccagt |
| | gcaccccgcg gtaacaccct |
| 5641 | ccaccttcac tttatgcctt gcacttactg tttcctctgc |
| | ccaggggttc tttgctccgt |
| 5701 | ctctactgtt tcaaatactg cccaacctca aagcccagct |
| | ccaaagctac ctcctctgtg |
| 5761 | aagaactcct tggaaatgat catctcagac tcctctattg |
| | gctgtcccag cacaagtgat |
| 5821 | cacgtttaac ttctgaaggc ctggacagaa tcttgagtgg |
| | gtccgccatt ccattccaag |
| 5881 | tcggccctca ccgtgcactt cctcttctcc cgccagattc |
| | ttcaaagtga cgcctccccc |
| 5941 | aggaagcggg ccccagaacc agtacgggaa cgtgctgtct |
| | ctccccacac ccacctcagg |
| 6001 | cctcggtaag aggcaccgcc cctccagcct atagctccgc |
| | cccagatccg gggctccacc |
| 6061 | cccactctcc tcatccctcc aatccgctgt gcgccaagcc |
| | ttctggagct cggaactccg |
| 6121 | cccccggggc ggggagtccc gcccagctat gagccccgcc |
| | tctagaacca gaccccgcct |
| 6181 | ccagggctca gagccacgcc cccaggaccc agagcctgaa |
| | gtcgtaatca agagcagaac |
| 6241 | ttcgccccag aactgaaggc ctcggcccta gatttagatt |
| | ccgccccagg gttcaaggcc |
| 6301 | gggttcctag acccagagtc cattcgcaga gcccaaaaca |
| | tcctcttccc gtgccccgcc |
| 6361 | gcgcggaccc ttagccttga ccgcccccat ctcttctgac |
| | cccgtcttac aatgcccctc |
| 6421 | tcaccaggac gcgcccagcg ttgggccgca ggcctggggg |
| | gcactgcccc gtcttatgga |
| 6481 | aacccgagca gcgacgtcca ggcggatgga gccttggggt |
| | cccggagccc gccgggagtg |
| 6541 | ggtgaatgac tgggagaggg aagggtcgtt ccccacatgg |
| | agggggttgg agcggtctgt |
| 6601 | ggcccgaata gtggactggg ccctggagga gagggggcat |
| | gactcggttc cccatcccca |
| 6661 | tccccaaacc cccaggccca gaagaagagg aaggggaggg |
| | ctatgaggaa cctgacagtg |
| 6721 | aggaggactc cgagttctat gagaacgact ccaaccttgg |
| | gcaggaccag ctctcccagg |
| 6781 | gtaaggctgc cctcccccgt ggccccccac ctctgcggtg |
| | gcctgtggac tcccatggac |
| 6841 | acccctcctt ctacaccaga tggcagcggc tacgagaacc |
| | ctgaggatga gcccctgggt |
| 6901 | cctgaggatg aagactcctt ctccaacggt aacttggggc |
| | ctttgtggga cctcagagac |
| 6961 | ttaggtgtaa ttgcagcgct gtgacactcc tagaagggga |
| | tccctggagt tctctctctt |
| 7021 | ctgccacagc tgagtcttat gagaacgagg atgaagagct |
| | gacccagccg gtcgccagga |
| 7081 | caatgggtgt gtgtgaggat ggcaacagtc caggggggag |
| | gcggaggaca cctggaggcc |
| 7141 | aggaggaata gtaacctccc tcttcccttt ccagacttcc |
| | tgagccctca tgggtcagcc |
| 7201 | tgggacccca gccgggaagc aacctccctg ggtgagagat |
| | gctttcaatc agactgcctt |
| 7261 | gcccagcttg ggtgacctgg cctcagctct gacaccagat |
| | ccaactttga cctgaccctg |
| 7321 | accccaaacc cgaacccaat cctgtgactc ctctcacctc |
| | aacactgagc cccatccccc |
| 7381 | atcctgagcc ccatccccca tcctgacccc caatatttac |
| | cccctcccta actgtgaata |
| 7441 | tcaacaccga tcccaatgca gtatcagcct ggacttgatc |
| | tccacctcac ctcagcccca |
| 7501 | gtgcagacct caacttggac cccagcttac tctgcagctt |
| | cttcatgact ctgactccga |
| 7561 | ctccctccag tttcttcttt ttctttttct tttttttgag |
| | acggagtctc cctctgttgc |
| 7621 | ccaggctgga gtgcagttgc cacctctgcc tcctaggttc |
| | aagcgattct catgcctcag |
| 7681 | cctcctgagt agctgggatt atagacgttt gccaccacac |
| | ctggctaatt tttgtatttt |
| 7741 | cagtagagac agggtttcgc catgttggcc agactggtct |
| | ccaactcctg gcctctagtg |
| 7801 | atctgcccgc ctttggcttc ccaaagtgct gggattacag |
| | gcatgagcca ccacgcccag |
| 7861 | cccagttctg ttcttgaccc cttccttagc cataatctaa |
| | cccatatcta accctgaccc |
| 7921 | tacagctaac tggggcccca aactcaatgc taaccaaatc |
| | accccttccc agcacagcat |
| 7981 | gggtaatgct cctcaccttc ctctgcccct cagtcttcct |
| | ccttaccgta ggctgtactt |
| 8041 | cccatgccct agcctccaat tctccatccc ccgcccaagc |
| | agggtcccag tcctatgagg |
| 8101 | atatgagagg aatcctgtat gcagcccccc agctccgctc |
| | cattcggggc cagcctggac |
| 8161 | ccaatcatga ggaaggtggg tgcttctgcc gctgtcccct |
| | gctgtcccct gggctgactt |
| 8221 | tgccttccag cctacttcca gtgccaccca tgttctcctc |
| | ctccctggtc ctatccagat |
| 8281 | gcagactctt atgagaacat ggataatccc gatgggccag |
| | acccagcctg gggaggaggg |
| 8341 | ggccgcatgg gcacctggag caccaggtga tcctcaggtg |
| | gccaggtgag ctgggactgc |
| 8401 | ccctagggaa agcggggagg gagggagata ggcacggatg |
| | gcagtggctg ctggctttca |
| 8461 | gggagggaga gggaacaggg ttcctagggc ctggtgggca |
| | gggggaggac tgctggaccc |
| 8521 | ctccccatca ccgtttcttc tgcatagcct ggatctcctc |
| | aagtccccaa gattcacacc |
| 8581 | tgactctgaa atctgaagac ctcgagcaga tgatgccaac |
| | ctctggagca atgttgctta |
| 8641 | ggatgtgtgc atgtgtgtaa gtgtgtgtgt gtgtgtgtgt |
| | gtgtatacat gccagtgaca |
| 8701 | cttccagtcc cctttgtatt ccttaaataa actcaatgag |
| | ctc |

An exemplary human CD72 amino acid sequence is set forth below (SEQ ID NO: 124; GenBank Accession No: NP_001773.1, Version 1, incorporated herein by reference):

| | |
|---|---|
| 1 | maeaityadl rfvkaplkks issrlgqdpg adddgeitye |
| | nvqvpavlgv psslassvlg |
| 61 | dkaavkseqp taswravtsp avgrilpcrt tclrylllgl |
| | lltclllgvt aiclgvrylq |
| 121 | vsqqlqqtnr vlevtnsslr qqlrlkitql gqsaedlqgs |
| | rrelaqsqea lqveqrahqa |
| 181 | aegqlqacqa drqktketlq seeqqrrale qklsnmenrl |
| | kpfftcgsad tccpsgwimh |
| 241 | qkscfyislt sknwqesqkq cetlssklat fseiypqshs |
| | yyflnsllpn ggsgnsywtg |
| 301 | lssnkdwklt ddtqrtrtya gsskcnkvhk twswwtlese |
| | scrsslpyic emtafrfpd |

An exemplary human CD72 nucleic acid sequence is set forth below (SEQ ID NO: 125; GenBank Accession No: NM_001782.2, Version 2, incorporated herein by reference):

| | |
|---|---|
| 1 | aattgctaag ccgtgcagtc acagagggaa cacagagcct |
| | agttgtaaac ggacagagac |
| 61 | gagaggggca agggaggaca gtggatgaca gggaagacga |
| | gtgggggcag agctgctcag |
| 121 | gaccatggct gaggccatca cctatgcaga tctgaggttt |
| | gtgaaggctc ccctgaagaa |
| 181 | gagcatctcc agccggttag gacaggaccc aggggctgat |
| | gatgatgggg aaatcaccta |
| 241 | cgagaatgtt caagtgcccg cagtcctagg ggtgccctca |
| | agcttggctt cttctgtact |
| 301 | aggggacaaa gcagcggtca agtcggagca gccaactgcg |
| | tcctggagag ccgtgacgtc |
| 361 | accagctgtc gggcggattc tcccctgccg cacaacctgc |
| | ctgcgatacc tcctgctcgg |
| 421 | cctgctcctc acctgcctgc tgttaggagt gaccgccatc |
| | tgcctgggag tgcgctatct |
| 481 | gcaggtgtct cagcagctcc agcagacgaa cagggttctg |
| | gaagtcacta acagcagcct |
| 541 | gaggcagcag ctccgcctca agataacgca gctgggacag |
| | agtgcagagg atctgcaggg |
| 601 | gtccaggaga gagctggcgc agagtcagga agcactacag |
| | gtggaacaga gggctcatca |
| 661 | ggcggccgaa gggcagctac aggcctgcca ggcagacaga |
| | cagaagacga aggagacctt |
| 721 | gcaaagtgag gagcaacaga ggagggcctt ggagcagaag |
| | ctgagcaaca tggagaacag |
| 781 | actgaagccc ttcttcacat gcggctcagc agacacctgc |
| | tgtccgtcgg gatggataat |
| 841 | gcatcagaaa agctgctttt acatctcact tacttcaaaa |
| | aattggcagg agagccaaaa |
| 901 | acaatgtgaa actctgtctt ccaagctggc cacattcagt |
| | gaaatttatc cacaatcaca |
| 961 | ctcttactac ttcttaaatt cactgttgcc aaatggtggt |
| | tcagggaatt catattggac |
| 1021 | tggcctcagc tctaacaagg attggaagtt gactgatgat |
| | acacaacgca ctaggactta |
| 1081 | tgctcaaagc tcaaaatgta acaaggtaca taaaacttgg |
| | tcatggtgga cactggagtc |
| 1141 | agagtcatgt agaagttctc ttccctacat ctgtgagatg |
| | acagctttca ggtttccaga |
| 1201 | ttaggacagt cctttgcact gagttgacac tcatgccaac |
| | aagaacctgt gcccctcctt |
| 1261 | cctaacctga ggcctggggt tcctcagacc atctccttca |
| | ttctgggcag tgcccagcca |
| 1321 | ccggctgacc cacacctgac acttccagcc agtctgctgc |
| | ctgctccctc ttcctgaaac |
| 1381 | tggactgttc ctgggaaaag ggtgaagcca cctctagaag |
| | ggactttggc ctccccccaa |
| 1441 | gaacttccca tggtagaatg gggtggggga ggagggcgca |
| | cgggctgagc ggataggggc |
| 1501 | ggcccggagc cagccaggca gttttattga aatcttttta |
| | aataattg |

An exemplary human FCRL1/3 amino acid sequence is set forth below (SEQ ID NO: 126; GenBank Accession No: Q96LA6.1, Version 1, incorporated herein by reference):

| | |
|---|---|
| 1 | mlprllllic aplcepaelf liaspshpte gspvtltckm |
| | pflqssdaqf qfcffrdtra |
| 61 | lgpgwssspk lqiaamwked tgsywceaqt maskvlrsrr |
| | sqinvhrvpv advsletqpp |
| 121 | ggqvmegdrl vlicsvamgt gditflwykg avglnlqskt |
| | qrsltaeyei psvresdaeq |
| 181 | yycvaengyg pspsglvsit vripvsrpil mlrapraqaa |
| | vedvlelhce alrgsppily |
| 241 | wfyheditlg srsapsggga sfnlslteeh sgnysceann |
| | glgaqrseav tlnftvptga |
| 301 | rsnhltsgvi egllstlgpa tvallfcygl krkigrrsar |
| | dplrslpspl pqeftylnsp |
| 361 | tpgqlqpiye nvnvvsgdev yslayynqpe qesvaaetlg |
| | thmedkvsld iysrlrkani |
| 421 | tdvdyedam |

An exemplary human FCRL1/3 nucleic acid sequence is set forth below (SEQ ID NO: 127; GenBank Accession No: NM_052938.4, Version 4, incorporated herein by reference):

| | |
|---|---|
| 1 | aacttccgat atcaacttcc tcaaacctct gatgagctgc |
| | tgctgctcga ctctgaggtg |
| 61 | cattcttttt ttgatgagag gcatctctag gtaccatccc |
| | tgacctggtc ctcatgctgc |
| 121 | cgaggctgtt gctgttgatc tgtgctccac tctgtgaacc |
| | tgccgagctg tttttgatag |
| 181 | ccagcccctc ccatcccaca gaggggagcc cagtgaccct |
| | gacgtgtaag atgccctttc |
| 241 | tacagagttc agatgcccag ttccagttct gctttttcag |
| | agacacccgg gccttgggcc |
| 301 | caggctggag cagctccccc aagctccaga tcgctgccat |
| | gtggaaagaa gacacagggt |
| 361 | catactggtg cgaggcacag acaatggcgt ccaaagtctt |
| | gaggagcagg agatcccaga |
| 421 | taaatgtgca cagggtccct gtcgctgatg tgagcttgga |
| | gactcagccc ccaggaggac |
| 481 | aggtgatgga gggagacagg ctggtcctca tctgctcagt |
| | tgctatgggc acaggagaca |
| 541 | tcaccttcct ttggtacaaa ggggctgtag gtttaaacct |
| | tcagtcaaag acccagcgtt |
| 601 | cactgacagc agagtatgag attccttcag tgagggagag |
| | tgatgctgag caatattact |
| 661 | gtgtagctga aaatggctat ggtcccagcc ccagtgggct |
| | ggtgagcatc actgtcagaa |
| 721 | tcccggtgtc tcgcccaatc ctcatgctca gggctcccag |
| | ggcccaggct gcagtggagg |
| 781 | atgtgctgga gcttcactgt gaggccctga gaggctctcc |
| | tccgatcctg tactggtttt |
| 841 | atcacgagga tatcaccctg gggagcaggt cggccccctc |
| | tggaggagga gcctccttca |
| 901 | acctttccct gactgaagaa cattctggaa actactcctg |
| | tgaggccaac aatggcctgg |
| 961 | gggcccagcg cagtgaggcg gtgacactca acttcacagt |
| | gcctactggg gccagaagca |
| 1021 | atcatcttac ctcaggagtc attgaggggc tgctcagcac |
| | ccttggtcca gccaccgtgg |
| 1081 | ccttattatt ttgctacggc ctcaaaagaa aaataggaag |
| | acgttcagcc agggatccac |
| 1141 | tcaggagcct tcccagccct ctaccccaag agttcaccta |
| | cctcaactca cctaccccag |
| 1201 | ggcagctaca gcctatatat gaaaatgtga atgttgtaag |
| | tggggatgag gtttattcac |
| 1261 | tggcgtacta taaccagccg gagcaggaat cagtagcagc |
| | agaaaccctg gggacacata |
| 1321 | tggaggacaa ggtttcctta gacatctatt ccaggctgag |
| | gaaagcaaac attacagatg |
| 1381 | tggactatga agatgctatg taaggttatg gaagattctg |
| | ctctttgaaa accatccatg |
| 1441 | accccaagcc tcaggcctga tatgttcttc agagatcctg |
| | gggcattagc tttccagtat |
| 1501 | acctcttctg gatgccattc tccatggcac tattccttca |
| | tctactgtga agtgaagttg |
| 1561 | gcgcagccct gaagaaacta cctaggagaa ctaatagaca |
| | caggagtgac agggactttg |
| 1621 | ttatcagaac cagattcctg ccggctcctt tgaaaacagg |
| | tcatattgtg ctcttctgtt |
| 1681 | tacaagagga aacaagatgg aataaaagaa attgggatct |
| | tgggttggag ggacagtgaa |
| 1741 | gcttagagca catgaactca aggttagtga ctctgcagga |
| | cttcacagag agagctgtgc |
| 1801 | ccatcattca gtccaagtgc tttctctgcc cagacagcac |
| | agaactccag ccccgctact |
| 1861 | tacatggatc atcgagtttc cacctaaaat atgattctat |
| | ttattttgag tcactgttac |
| 1921 | caaattagaa ctaaaacaaa gttacataaa aagttattgt |
| | gactccactt aattttagtg |
| 1981 | acgtattttt gtatatatag gccaacctat accacatcca |
| | aaattatgta tctattacag |
| 2041 | cccctagaag ctttataaat acagtgtgtc ttcttttatt |
| | cacaaaattt ttgaaatcgt |
| 2101 | ggtaatatgg tttgaaacct gtatcttaat tatttttttt |
| | ttaaattgag acagggtctc |
| 2161 | actctgtcac tcaatctgga atgcagtggc acaatcttgc |
| | ctcactgcaa cgcctgcctc |
| 2221 | tcaggctcaa gcaaacctct cacctcagcc tgctgagtag |
| | ctgggactac aggcacatgc |
| 2281 | caccaaactt ggccattttt tgtcttacgt agagacaaga |
| | tttcaccgtt ttgcccaggc |
| 2341 | tggtctcaaa ctcctgggct caagcaatgt attgaatttt |
| | aaaataacca ggcactcact |
| 2401 | cttatgaatt aataaacatt tggaggtata taaagtaaaa |
| | agttaaagtc tttcctgtaa |
| 2461 | gttaacacaa atgttaacta ttgttaaaaa ctttacaggt |
| | agctctctag atatttttct |
| 2521 | atttttgtat gtatacttat gcatacatgt aagtatataa |
| | acatttagaa gtgtacctat |
| 2581 | ctaacaaact attatgaaat actttcaaat ctgtaaatag |
| | atctattata ctattttaaa |
| 2641 | agtctctata gtagtgtgtt atatagataa atcataactt |
| | ttttcttttt ttattgtagt |
| 2701 | aaatatgcac aacataaaat tgatcatttt aaccattttt |
| | aagtgtacaa ttcagtggca |
| 2761 | ttaagtacta tcataatata ttttaatcct tctcatcact |
| | ggtggacatt aaggagactc |
| 2821 | tcaaaaaatt catattataa aaacaaagtt caaacaaatg |
| | tctttgtact agcatattat |
| 2881 | ggcactcctg ctggattatc tgaaggataa atttgtaaat |
| | ctagtattgc tagattatgc |
| 2941 | atattaaata ttcttgttaa atagtcttca atgtctctca |
| | ggtaaggctg tatcaattta |
| 3001 | tatcttcacc aacaacgtct gggaaatcag tttgtggggt |
| | gtattactta gttttcacat |
| 3061 | tgctaataaa gacatatcca agactgggta atttataaaa |
| | aaaaaaaaaa aaaaaa |

An exemplary human MS4A1 amino acid sequence is set forth below (SEQ ID NO: 128; GenBank Accession No: P11836.1, Version 1, incorporated herein by reference):

| | |
|---|---|
| 1 | mttprnsvng tfpaepmkgp iamqsgpkpl frrmsslvgp |
| | tqsffmresk tlgavqimng |
| 61 | lfhialggll mipagiyapi cvtvwyplwg gimyiisgsl |
| | laateknsrk clvkgkmimn |
| 121 | slslfaaisg milsimdiln ikishflkme slnfirahtp |
| | yiniyncepa npseknspst |
| 181 | qycysiqslf lgilsvmlif affqelviag ivenewkrtc |
| | srpksnivll saeekkeqti |
| 241 | eikeevvglt etssqpknee dieiipiqee eeeetetnfp |
| | eppqdqessp iendssp |

An exemplary human MS4A1 nucleic acid sequence is set forth below (SEQ ID NO: 129; GenBank Accession No: NM_152866.2, Version 2, incorporated herein by reference):

| | |
|---|---|
| 1 | gtctatcagc gatttcatct tcaggcctgg actacaccac |
| | tcaccctccc agtgtgcttg |
| 61 | agaaacaaac tgcacccact gaactccgca gctagcatcc |
| | aaatcagccc ttgagatttg |
| 121 | aggccttgga gactcagatc ctgaacaaga gagaacaaaa |
| | tctctacttt gatggaactt |
| 181 | ccattctgtg gggaagagac tgacaataag caattaaata |
| | aataagaact cagcagtagg |
| 241 | ccttgcctca gatccaaggt cactcggaag aggccatgtc |
| | taccctcaat gacactcatg |
| 301 | gaggaaatgc tgagagaagc attcagatgc atgacacaag |
| | gtaagactgc caaaaatctt |
| 361 | gttcttgctc tcctcatttt gttatttgtt ttatttttag |
| | gagttttgag agcaaaatga |
| 421 | caacacccag aaattcagta aatgggactt tcccggcaga |
| | gccaatgaaa ggccctattg |
| 481 | ctatgcaatc tggtccaaaa ccactcttca ggaggatgtc |
| | ttcactggtg ggccccacgc |
| 541 | aaagcttctt catgagggaa tctaagactt tgggggctgt |
| | ccagattatg aatgggctct |
| 601 | tccacattgc cctggggggt cttctgatga tcccagcagg |
| | gatctatgca cccatctgtg |
| 661 | tgactgtgtg gtaccctctc tggggaggca ttatgtatat |
| | tatttccgga tcactcctgg |
| 721 | cagcaacgga gaaaaactcc aggaagtgtt tggtcaaagg |
| | aaaaatgata atgaattcat |
| 781 | tgagcctctt tgctgccatt tctggaatga ttctttcaat |
| | catggacata cttaatatta |
| 841 | aaatttccca ttttttaaaa atggagagtc tgaattttat |
| | tagagctcac acaccatata |
| 901 | ttaacatata caactgtgaa ccagctaatc cctctgagaa |
| | aaactcccca tctacccaat |
| 961 | actgttacag catacaatct ctgttcttgg gcattttgtc |
| | agtgatgctg atctttgcct |
| 1021 | tcttccagga acttgtaata gctggcatcg ttgagaatga |
| | atggaaaaga acgtgctcca |
| 1081 | gacccaaatc taacatagtt ctcctgtcag cagaagaaaa |
| | aaaagaacag actattgaaa |
| 1141 | taaaagaaga agtggttggg ctaactgaaa catcttccca |
| | accaaagaat gaagaagaca |
| 1201 | ttgaaattat tccaatccaa gaagaggaag aagaagaaac |
| | agagacgaac tttccagaac |
| 1261 | ctccccaaga tcaggaatcc tcaccaatag aaaatgacag |
| | ctctccttaa gtgatttctt |
| 1321 | ctgttttctg tttccttttt taaacattag tgttcatagc |
| | ttccaagaga catgctgact |
| 1381 | ttcatttctt gaggtactct gcacatacgc accacatctc |
| | tatctggcct ttgcatggag |
| 1441 | tgaccatagc tccttctctc ttacattgaa tgtagagaat |
| | gtagccattg tagcagcttg |
| 1501 | tgttgtcacg cttcttcttt tgagcaactt tcttacactg |
| | aagaaaggca gaatgagtgc |
| 1561 | ttcagaatgt gatttcctac taacctgttc cttggatagg |
| | ctttttagta tagtattttt |
| 1621 | ttttgtcatt ttctccatca acaaccaggg agactgcacc |
| | tgatggaaaa gatatatgac |
| 1681 | tgcttcatga cattcctaaa ctatcttttt tttattccac |
| | atctacgttt ttggtggagt |
| 1741 | cccttttgca tcattgtttt aaggatgata aaaaaaaata |
| | acaactaggg acaatacaga |
| 1801 | acccattcca tttatctttc tacagggctg acattgtggc |
| | acattcttag agttaccaca |
| 1861 | ccccatgagg gaagctctaa atagccaaca cccatctgtt |
| | ttttgtaaaa acagcatagc |
| 1921 | ttatacatgg acatgtctct gccttaactt ttcctaactc |
| | ccactctagg ctattgtttg |
| 1981 | catgtctacc tacttttagc cattatgcga gaaaagaaaa |
| | aaatgaccat agaaaatgcc |
| 2041 | accatgaggt gcccaaattt caaataataa ttaacattta |
| | gttatattta taatttccag |
| 2101 | atgacaaagt atttcatcaa ataacttcat ttgatgttcc |
| | atgatcaaga aagaatccct |
| 2161 | atctctattt tacaagtaat tcaaagaggc caaataactt |
| | gtaaacaaga aaaggtaact |
| 2221 | tgtcaacagt cataactagt aattatgaga gccttgtttc |
| | ataaccaggt cttcttactc |
| 2281 | aaatcctgtg atgtttgaaa taaccaaatt gtctctccaa |
| | tgtctgcata aactgtgaga |
| 2341 | gccaagtcaa cagcttttat caagaattta ctctctgacc |
| | agcaataaac aagcactgag |
| 2401 | agacacagag agccagattc agattttacc catggggata |
| | aaaagactca gactttcacc |
| 2461 | acatttggaa aactacttgc atcataaata tataataact |
| | ggtagtttat atgaagcaga |
| 2521 | cactaagtgc tatagacact ctcagaatat catacttgga |
| | aacaatgtaa ttaaaatgcc |
| 2581 | gaatctgagt caacagctgc cctacttttc aattcagata |
| | tactagtacc ttacctagaa |
| 2641 | ataatgttaa cctagggtga agtcactata atctgtagtc |
| | tattatttgg gcatttgcta |
| 2701 | catgatgagt gctgccagat tgtggcaggt aaagagacaa |
| | tgtaatttgc actccctatg |
| 2761 | atatttctac atttttagcg accactagtg gaagacattc |
| | cccaaaatta gaaaaaaagg |
| 2821 | agatagaaga tttctgtcta tgtaaagttc tcaaaatttg |
| | ttctaaatta ataaaactat |
| 2881 | ctttgtgttc ttttctgcaa cagatgattc caacatgggt |
| | gtttgtctat tcttctttac |
| 2941 | tcttgaaaca ttagaccatg ggaggctctt acagccttga |
| | gttgatattt atacaaccca |
| 3001 | aatctaggtt tgaacggtga ggtgtcaggt catcaaatat |
| | tcatgtctat atagtcttac |
| 3061 | acaggttctc aaaaaaaatg ttcatgggat aggtcattga |
| | taatggattc cttattctga |
| 3121 | gaactccaga cgactgaaat atatgagaga aggaaaagga |
| | catagtagga gcaggcctga |
| 3181 | gaaaaaaatg aaagtcagaa atctttaaaa aaatacaaga |
| | tcttatttct atcttatttt |
| 3241 | ttctcctctt ctgaaatata tatgaggatt cctctccaaa |
| | cccatggttt ctctaagaat |
| 3301 | tttgagtcat ttgtatgacc tcaaataatt agttttagct |
| | gacctcacat aactccttat |
| 3361 | aataggagac atctttaatg tctgctatta aagaaggatg |
| | aaaattccta tgaccttctc |
| 3421 | cccgattatc cctttggcaa tatagagtca aataataaca |
| | ttgaccaata gtaaacatgc |
| 3481 | tttgccaaga agtagaagat atattctcta gccttagttt |
| | ttcctcccaa tttgcatttt |
| 3541 | tgtaaaaata atgttgtatc cacaaaggaa ataaacttta |
| | aaaacccaag tgca |

An exemplary human CTLA4 amino acid sequence is set forth below (SEQ ID NO: 130; GenBank Accession No: AAL07473.1, Version 1, incorporated herein by reference):

| | |
|---|---|
| 1 | maclgfqrhk aqlnlatrtw pctllffllf ipvfckamhv |
| | aqpavvlass rgiasfvcey |
| 61 | aspgkatevr vtvlrqadsq vtevcaatym mgneltfldd |
| | sictgtssgn qvnltiqglr |
| 121 | amdtglyick velmypppyy lgigngtqiy vidpepcpds |
| | dfllwilaav ssglffysfl |
| 181 | ltavslskml kkrsplttgv yvkmpptepe cekqfqpyfi |
| | pin |

An exemplary human CTLA4 nucleic acid sequence is set forth below (SEQ ID NO: 131; GenBank Accession No: AF414120.1, Version 1, incorporated herein by reference):

| | |
|---|---|
| 1 | cttctgtgtg tgcacatgtg taatacatat ctgggatcaa |
| | agctatctat ataaagtcct |
| 61 | tgattctgtg tgggttcaaa cacatttcaa agcttcagga |
| | tcctgaaagg ttttgctcta |
| 121 | cttcctgaag acctgaacac cgctcccata aagccatggc |
| | ttgccttgga tttcagcggc |
| 181 | acaaggctca gctgaacctg gctaccagga cctggccctg |
| | cactctcctg ttttttcttc |
| 241 | tcttcatccc tgtcttctgc aaagcaatgc acgtggccca |
| | gcctgctgtg gtactggcca |
| 301 | gcagccgagg catcgccagc tttgtgtgtg agtatgcatc |
| | tccaggcaaa gccactgagg |
| 361 | tccgggtgac agtgcttcgg caggctgaca gccaggtgac |
| | tgaagtctgt gcggcaacct |
| 421 | acatgatggg gaatgagttg accttcctag atgattccat |
| | ctgcacgggc acctccagtg |
| 481 | gaaatcaagt gaacctcact atccaaggac tgagggccat |
| | ggacacggga ctctacatct |
| 541 | gcaaggtgga gctcatgtac ccaccgccat actacctggg |
| | cataggcaac ggaacccaga |
| 601 | tttatgtaat tgatccagaa ccgtgcccag attctgactt |
| | cctcctctgg atccttgcag |
| 661 | cagttagttc ggggttgttt ttttatagct ttctcctcac |
| | agctgtttct ttgagcaaaa |
| 721 | tgctaaagaa aagaagccct cttacaacag gggtctatgt |
| | gaaaatgccc ccaacagagc |
| 781 | cagaatgtga aaagcaattt cagccttatt ttattcccat |
| | caattgagaa accattatga |
| 841 | agaagagagt ccatatttca atttccaaga gctgaggcaa |
| | ttctaacttt tttgctatcc |
| 901 | agctattttt atttgtttgt gcatttgggg ggaattcatc |
| | tctctttaat ataaagttgg |
| 961 | atgcggaacc caaattacgt gtactacaat ttaaagcaaa |
| | ggagtagaaa gacagagctg |
| 1021 | ggatgtttct gtcacatcag ctccactttc agtgaaagca |
| | tcacttggga ttaatatggg |
| 1081 | gatgcagcat tatgatgtgg gtcaaggaat taagttaggg |
| | aatggcacag cccaaagaag |
| 1141 | gaaaaggcag ggagcgaggg agaagactat attgtacaca |
| | ccttatattt acgtatgaga |
| 1201 | cgtttatagc cgaaatgatc ttttcaagtt aaattttatg |
| | ccttttattt cttaaacaaa |
| 1261 | tgtatgatta catcaaggct tcaaaaatac tcacatggct |
| | atgttttagc cagtgatgct |
| 1321 | aaaggttgta ttgcatatat acatatatat atatatatat |
| | atatatatat atatatatat |
| 1381 | atatatatat tttaatttga tagtattgtg catagagcca |
| | cgtatgtttt tgtgtatttg |
| 1441 | ttaatggttt gaatataaac actatatggc agtgtctttc |
| | caccttgggt cccagggaag |
| 1501 | ttttgtggag gagctcagga cactaataca ccaggtagaa |
| | cacaaggtca tttgctaact |
| 1561 | agcttggaaa ctggatgagg tcatagcagt gcttgattgc |
| | gtggaattgt gctgagttgg |
| 1621 | tgttgacatg tgctttgggg cttttacacc agttcctttc |
| | aatggtttgc aaggaagcca |
| 1681 | cagctggtgg tatctgagtt gacttgacag aacactgtct |
| | tgaagacaat ggcttactcc |
| 1741 | aggagaccca caggtatgac cttctaggaa gctccagttc |
| | gatgggccca attcttacaa |
| 1801 | acatgtggtt aatgccatgg acagaagaag gcagcaggtg |
| | gcagaatggg gtgcatgaag |
| 1861 | gtttctgaaa attaacactg cttgtgtttt taactcaata |
| | ttttccatga aaatgcaaca |
| 1921 | acatgtataa tatttttaat taaataaaaa tctgtggtgg |
| | tcgttttaaa aaaaaaaaaa |
| 1981 | aaaaaaaaaa aaaaaaaaaa aaaaaaaaaa aaaaaaaaaa |
| | aaaaa |

An exemplary human LAG3 amino acid sequence is set forth below (SEQ ID NO: 132; GenBank Accession No: AAH52589.1, Version 1, incorporated herein by reference):

| | |
|---|---|
| 1 | mweaqflgll flqplwvapv kplqpgaevp vvwaqegapa |
| | qlpcsptipl qdlsllrrag |
| 61 | vtwqhqpdsg ppaaapghpl apgphpaaps swgprprryt |
| | vlsvgpgglr sgrlplqprv |
| 121 | qldergrqrg dfslwlrpar radageyraa vhlrdralsc |
| | rlrlrlgqas mtasppgslr |
| 181 | asdwvilncs fsrpdrpasv hwfrnrgqgr vpvresphhh |
| | laesflflpq vspmdsgpwg |
| 241 | ciltyrdgfn vsimynltvl glepptpltv yagagsrvgl |
| | pcrlpagvgt rsfltakwtp |
| 301 | pgggpdllvt gdngdftlrl edvsqaqagt ytchihlqeq |
| | qlnatvtlai itgqpqvgke |

An exemplary human LAG3 nucleic acid sequence is set forth below (SEQ ID NO: 133; GenBank Accession No: NM_002286.5, Version 5, incorporated herein by reference):

| | |
|---|---|
| 1 | acaggggtga aggcccagag accagcagaa cggcatccca |
| | gccacgacgg ccactttgct |
| 61 | ctgtctgctc tccgccacgg ccctgctctg ttccctggga |
| | cacccccgcc cccacctcct |
| 121 | caggctgcct gatctgccca gctttccagc tttcctctgg |
| | attccggcct ctggtcatcc |
| 181 | ctccccaccc tctctccaag gccctctcct ggtctccctt |
| | cttctagaac cccttcctcc |
| 241 | acctccctct ctgcagaact tctcctttac cccccacccc |
| | ccaccactgc cccctttcct |
| 301 | tttctgacct ccttttggag ggctcagcgc tgcccagacc |
| | ataggagaga tgtgggaggc |
| 361 | tcagttcctg ggcttgctgt ttctgcagcc gctttgggtg |
| | gctccagtga agcctctcca |
| 421 | gccaggggct gaggtcccgg tggtgtgggc ccaggagggg |
| | gctcctgccc agctcccctg |
| 481 | cagccccaca atccccctcc aggatctcag ccttctgcga |
| | agagcagggg tcacttggca |
| 541 | gcatcagcca gacagtggcc cgcccgctgc cgcccccggc |
| | catcccctgg cccccggccc |
| 601 | tcacccggcg gcgccctcct cctgggggcc caggccccgc |
| | cgctacacgg tgctgagcgt |
| 661 | gggtcccgga ggcctgcgca gcgggaggct gcccctgcag |
| | ccccgcgtcc agctggatga |
| 721 | gcgcggccgg cagcgcgggg acttctcgct atggctgcgc |
| | ccagcccggc gcgcggacgc |
| 781 | cggcgagtac cgcgccgcgg tgcacctcag ggaccgcgcc |
| | ctctcctgcc gcctccgtct |
| 841 | gcgcctgggc caggcctcga tgactgccag ccccccagga |
| | tctctcagag cctccgactg |
| 901 | ggtcattttg aactgctcct tcagccgccc tgaccgccca |
| | gcctctgtgc attggttccg |
| 961 | gaaccggggc cagggccgag tccctgtccg ggagtccccc |
| | catcaccact tagcggaaag |
| 1021 | cttcctcttc ctgccccaag tcagccccat ggactctggg |
| | ccctggggct gcatcctcac |
| 1081 | ctacagagat ggcttcaacg tctccatcat gtataacctc |
| | actgttctgg gtctggagcc |
| 1141 | cccaactccc ttgacagtgt acgctggagc aggttccagg |
| | gtggggctgc cctgccgcct |
| 1201 | gcctgctggt gtggggaccc ggtctttcct cactgccaag |
| | tggactcctc ctgggggagg |
| 1261 | ccctgacctc ctggtgactg gagacaatgg cgactttacc |
| | cttcgactag aggatgtgag |
| 1321 | ccaggcccag gctgggacct acacctgcca tatccatctg |
| | caggaacagc agctcaatgc |
| 1381 | cactgtcaca ttggcaatca tcacagtgac tcccaaatcc |
| | tttgggtcac ctggatccct |
| 1441 | ggggaagctg ctttgtgagg tgactccagt atctggacaa |
| | gaacgctttg tgtggagctc |
| 1501 | tctggacacc ccatcccaga ggagtttctc aggaccttgg |
| | ctggaggcac aggaggccca |
| 1561 | gctcctttcc cagccttggc aatgccagct gtaccagggg |
| | gagaggcttc ttggagcagc |
| 1621 | agtgtacttc acagagctgt ctagcccagg tgcccaacgc |
| | tctgggagag ccccaggtgc |
| 1681 | cctcccagca ggccacctcc tgctgtttct catccttggt |
| | gtcctttctc tgctcctttt |
| 1741 | ggtgactgga gcctttggct ttcacctttg gagaagacag |
| | tggcgaccaa gacgattttc |
| 1801 | tgccttagag caagggattc accctccgca ggctcagagc |
| | aagatagagg agctggagca |
| 1861 | agaaccggag ccggagccgg agccggaacc ggagcccgag |
| | cccgagcccg agccggagca |
| 1921 | gctctgacct ggagctgagg cagccagcag atctcagcag |
| | cccagtccaa ataaactccc |
| 1981 | tgtcagcagc aaaaa |

An exemplary human FCRL1 amino acid sequence is set forth below (SEQ ID NO: 134; GenBank Accession No: Q96LA6.1, Version 1, incorporated herein by reference):

| | |
|---|---|
| 1 | mlprllllic aplcepaelf liaspshpte gspvtltckm |
| | pflqssdaqf qfcffrdtra |
| 61 | lgpgwssspk lqiaamwked tgsywceaqt maskvlrsrr |
| | sqinvhrvpv advsletqpp |
| 121 | ggqvmegdrl vlicsvamgt gditflwykg avglnlqskt |
| | qrsltaeyei psvresdaeq |
| 181 | yycvaengyg pspsglvsit vripvsrpil mlrapraqaa |
| | vedvlelhce alrgsppily |
| 241 | wfyheditlg srsapsggga sfnlslteeh sgnysceann |
| | glgaqrseav tlnftvptga |
| 301 | rsnhltsgvi egllstlgpa tvallfcygl krkigrrsar |
| | dplrslpspl pqeftylnsp |
| 361 | tpgqlqpiye nvnvvsgdev yslayynqpe qesvaaetlg |
| | thmedkvsld iysrlrkani |
| 421 | tdvdyedam |

An exemplary human FCRL1 nucleic acid sequence is set forth below (SEQ ID NO: 135; GenBank Accession No: NM_052938.4, Version 4, incorporated herein by reference):

| | |
|---|---|
| 1 | aacttccgat atcaacttcc tcaaacctct gatgagctgc |
| | tgctgctcga ctctgaggtg |
| 61 | cattcttttt ttgatgagag gcatctctag gtaccatccc |
| | tgacctggtc ctcatgctgc |
| 121 | cgaggctgtt gctgttgatc tgtgctccac tctgtgaacc |
| | tgccgagctg tttttgatag |
| 181 | ccagcccctc ccatcccaca gaggggagcc cagtgaccct |
| | gacgtgtaag atgccctttc |
| 241 | tacagagttc agatgcccag ttccagttct gctttttcag |
| | agacacccgg gccttgggcc |
| 301 | caggctggag cagctccccc aagctccaga tcgctgccat |
| | gtggaaagaa gacacagggt |
| 361 | catactggtg cgaggcacag acaatggcgt ccaaagtctt |
| | gaggagcagg agatcccaga |
| 421 | taaatgtgca cagggtccct gtcgctgatg tgagcttgga |
| | gactcagccc ccaggaggac |
| 481 | aggtgatgga gggagacagg ctggtcctca tctgctcagt |
| | tgctatgggc acaggagaca |
| 541 | tcaccttcct ttggtacaaa ggggctgtag gtttaaacct |
| | tcagtcaaag acccagcgtt |
| 601 | cactgacagc agagtatgag attccttcag tgagggagag |
| | tgatgctgag caatattact |
| 661 | gtgtagctga aaatggctat ggtcccagcc ccagtgggct |
| | ggtgagcatc actgtcagaa |
| 721 | tcccggtgtc tcgcccaatc ctcatgctca gggctcccag |
| | ggcccaggct gcagtggagg |
| 781 | atgtgctgga gcttcactgt gaggccctga gaggctctcc |
| | tccgatcctg tactggtttt |
| 841 | atcacgagga tatcaccctg gggagcaggt cggccccctc |
| | tggaggagga gcctccttca |
| 901 | acctttccct gactgaagaa cattctggaa actactcctg |
| | tgaggccaac aatggcctgg |
| 961 | gggcccagcg cagtgaggcg gtgacactca acttcacagt |
| | gcctactggg gccagaagca |
| 1021 | atcatcttac ctcaggagtc attgaggggc tgctcagcac |
| | ccttggtcca gccaccgtgg |
| 1081 | ccttattatt ttgctacggc ctcaaaagaa aaataggaag |
| | acgttcagcc agggatccac |
| 1141 | tcaggagcct tcccagccct ctaccccaag agttcaccta |
| | cctcaactca cctaccccag |
| 1201 | ggcagctaca gcctatatat gaaaatgtga atgttgtaag |
| | tggggatgag gtttattcac |
| 1261 | tggcgtacta taaccagccg gagcaggaat cagtagcagc |
| | agaaaccctg gggacacata |
| 1321 | tggaggacaa ggtttcctta gacatctatt ccaggctgag |
| | gaaagcaaac attacagatg |
| 1381 | tggactatga agatgctatg taaggttatg gaagattctg |
| | ctctttgaaa accatccatg |
| 1441 | accccaagcc tcaggcctga tatgttcttc agagatcctg |
| | gggcattagc tttccagtat |
| 1501 | acctcttctg gatgccattc tccatggcac tattccttca |
| | tctactgtga agtgaagttg |
| 1561 | gcgcagccct gaagaaacta cctaggagaa ctaatagaca |
| | caggagtgac agggactttg |
| 1621 | ttatcagaac cagattcctg ccggctcctt tgaaaacagg |
| | tcatattgtg ctcttctgtt |
| 1681 | tacaagagga aacaagatgg aataaaagaa attgggatct |
| | tgggttggag ggacagtgaa |
| 1741 | gcttagagca catgaactca aggttagtga ctctgcagga |
| | cttcacagag agagctgtgc |
| 1801 | ccatcattca gtccaagtgc tttctctgcc cagacagcac |
| | agaactccag ccccgctact |
| 1861 | tacatggatc atcgagtttc cacctaaaat atgattctat |
| | ttattttgag tcactgttac |
| 1921 | caaattagaa ctaaaacaaa gttacataaa aagttattgt |
| | gactccactt aattttagtg |
| 1981 | acgtattttt gtatatatag gccaacctat accacatcca |
| | aaattatgta tctattacag |
| 2041 | cccctagaag ctttataaat acagtgtgtc ttcttttatt |
| | cacaaaattt ttgaaatcgt |
| 2101 | ggtaatatgg tttgaaacct gtatcttaat tatttttttt |
| | ttaaattgag acagggtctc |
| 2161 | actctgtcac tcaatctgga atgcagtggc acaatcttgc |
| | ctcactgcaa cgcctgcctc |
| 2221 | tcaggctcaa gcaaacctct cacctcagcc tgctgagtag |
| | ctgggactac aggcacatgc |
| 2281 | caccaaactt ggccattttt tgtcttacgt agagacaaga |
| | tttcaccgtt ttgcccaggc |
| 2341 | tggtctcaaa ctcctgggct caagcaatgt attgaatttt |
| | aaaataacca ggcactcact |
| 2401 | cttatgaatt aataaacatt tggaggtata taaagtaaaa |
| | agttaaagtc tttcctgtaa |
| 2461 | gttaacacaa atgttaacta ttgttaaaaa ctttacaggt |
| | agctctctag atatttttct |
| 2521 | atttttgtat gtatacttat gcatacatgt aagtatataa |
| | acatttagaa gtgtacctat |
| 2581 | ctaacaaact attatgaaat actttcaaat ctgtaaatag |
| | atctattata ctattttaaa |
| 2641 | agtctctata gtagtgtgtt atatagataa atcataactt |
| | ttttcttttt ttattgtagt |
| 2701 | aaatatgcac aacataaaat tgatcatttt aaccattttt |
| | aagtgtacaa ttcagtggca |
| 2761 | ttaagtacta tcataatata ttttaatcct tctcatcact |
| | ggtggacatt aaggagactc |
| 2821 | tcaaaaaatt catattataa aaacaaagtt caaacaaatg |
| | tctttgtact agcatattat |
| 2881 | ggcactcctg ctggattatc tgaaggataa atttgtaaat |
| | ctagtattgc tagattatgc |
| 2941 | atattaaata ttcttgttaa atagtcttca atgtctctca |
| | ggtaaggctg tatcaattta |
| 3001 | tatcttcacc aacaacgtct gggaaatcag tttgtggggt |
| | gtattactta gttttcacat |
| 3061 | tgctaataaa gacatatcca agactgggta atttataaaa |
| | aaaaaaaaaa aaaaaa |

An exemplary human FCRL3 amino acid sequence is set forth below (SEQ ID NO: 136; GenBank Accession No: AAH28933.1, Version 1, incorporated herein by reference):

| | |
|---|---|
| 1 | mllwllllil tpgreqsgva pkavlllnpp wstafkgekv |
| | alicssishs laqgdtywyh |
| 61 | dekllkikhd kiqitepgny qcktrgssls davhvefspd |
| | wlilqalhpv fegdnvilrc |
| 121 | qgkdnknthq kvyykdgkql pnsynlekit vnsysrdnsk |
| | yhctayrkfy ildievtskp |
| 181 | lniqvqelfl hpvlrassst piegspmtlt cetqlspqrp |
| | dvqlqfslfr dsqtlglgws |
| 241 | rsprlqipam wtedsgsywc evetvthsik krslrsqirv |
| | qrvpvsnvnl eirptggqli |
| 301 | egenmvlics vaqgsgtvtf swhkegrvrs lgrktqrsll |
| | aelhvltvke sdagryycaa |
| 361 | dnvhspilst wirvtvripv shpvltfrap rahtvvgdll |
| | elhceslrgs ppilyrfyhe |
| 421 | dvtlgnssap sgggasfnls ltaehsgnys cdadnglgaq |
| | hshgvslrvt vpvsrpvltl |
| 481 | rapgaqavvg dllelhcesl rgsfpilywf yheddtlgni |
| | sahsgggasf nlslttehsg |
| 541 | nysceadngl gaqhskvvtl nvtgtsrnrt gltaagitgl |
| | vlsilvlaaa aallhyarar |
| 601 | rkpgglsatg tsshspsecq epsssrpsri dpqepthskp |
| | lapmelepmy snvnpgdsnp |
| 661 | iysqiwsiqh tkensancpm mhqeheeltv lyselkkthp |
| | ddsageassr graheeddee |
| 721 | nyenilnprk nkvqdfpcic nt |

An exemplary human FCRL3 nucleic acid sequence is set forth below (SEQ ID NO: 137; GenBank Accession No: NM_052939.3, Version 3, incorporated herein by reference):

| | |
|---|---|
| 1 | agtgaagggg tttcccatat gaaaaataca gaaagaatta |
| | tttgaatact agcaaataca |
| 61 | caacttgata tttctagaga acccaggcac agtcttggag |
| | acattactcc tgagagactg |
| 121 | cagctgatgg aagatgagcc ccaacttcta aaaatgtatc |
| | actaccggga ttgagataca |
| 181 | aacagcattt aggaaggtct catctgagta gcagcttcct |
| | gccctccttc ttggagataa |
| 241 | gtcgggcttt tggtgagaca gactttccca accctctgcc |
| | cggccggtgc ccatgcttct |
| 301 | gtggctgctg ctgctgatcc tgactcctgg aagagaacaa |
| | tcaggggtgg ccccaaaagc |
| 361 | tgtacttctc ctcaatcctc catggtccac agccttcaaa |
| | ggagaaaaag tggctctcat |
| 421 | atgcagcagc atatcacatt ccctagccca gggagacaca |
| | tattggtatc acgatgagaa |
| 481 | gttgttgaaa ataaaacatg acaagatcca aattacagag |
| | cctggaaatt accaatgtaa |
| 541 | gacccgagga tcctccctca gtgatgccgt gcatgtggaa |
| | ttttcacctg actggctgat |
| 601 | cctgcaggct ttacatcctg tctttgaagg agacaatgtc |
| | attctgagat gtcaggggaa |
| 661 | agacaacaaa aacactcatc aaaaggttta ctacaaggat |
| | ggaaaacagc ttcctaatag |
| 721 | ttataattta gagaagatca cagtgaattc agtctccagg |
| | gataatagca aatatcattg |
| 781 | tactgcttat aggaagtttt acatacttga cattgaagta |
| | acttcaaaac ccctaaatat |
| 841 | ccaagttcaa gagctgtttc tacatcctgt gctgagagcc |
| | agctcttcca cgcccataga |
| 901 | ggggagtccc atgaccctga cctgtgagac ccagctctct |
| | ccacagaggc cagatgtcca |
| 961 | gctgcaattc tccctcttca gagatagcca gaccctcgga |
| | ttgggctgga gcaggtcccc |
| 1021 | cagactccag atccctgcca tgtggactga agactcaggg |
| | tcttactggt gtgaggtgga |
| 1081 | gacagtgact cacagcatca aaaaaaggag cctgagatct |
| | cagatacgtg tacagagagt |
| 1141 | ccctgtgtct aatgtgaatc tagagatccg gcccaccgga |
| | gggcagctga ttgaaggaga |
| 1201 | aaatatggtc cttatttgct cagtagccca gggttcaggg |
| | actgtcacat tctcctggca |
| 1261 | caaagaagga agagtaagaa gcctgggtag aaagacccag |
| | cgttccctgt tggcagagct |
| 1321 | gcatgttctc accgtgaagg agagtgatgc agggagatac |
| | tactgtgcag ctgataacgt |
| 1381 | tcacagcccc atcctcagca cgtggattcg agtcaccgtg |
| | agaattccgg tatctcaccc |
| 1441 | tgtcctcacc ttcagggctc ccagggccca cactgtggtg |
| | ggggacctgc tggagcttca |
| 1501 | ctgtgagtcc ctgagaggct ctcccccgat cctgtaccga |
| | ttttatcatg aggatgtcac |
| 1561 | cctggggaac agctcagccc cctctggagg aggagcctcc |
| | ttcaacctct ctctgactgc |
| 1621 | agaacattct ggaaactact cctgtgatgc agacaatggc |
| | ctgggggccc agcacagtca |
| 1681 | tggagtgagt ctcagggtca cagttccggt gtctcgcccc |
| | gtcctcaccc tcagggctcc |
| 1741 | cggggcccag gctgtggtgg gggacctgct ggagcttcac |
| | tgtgagtccc tgagaggctc |
| 1801 | cttcccgatc ctgtactggt tttatcacga ggatgacacc |
| | ttggggaaca tctcggccca |
| 1861 | ctctggagga ggggcatcct tcaacctctc tctgactaca |
| | gaacattctg gaaactactc |
| 1921 | atgtgaggct gacaatggcc tgggggccca gcacagtaaa |
| | gtggtgacac tcaatgttac |
| 1981 | aggaacttcc aggaacagaa caggccttac cgctgcggga |
| | atcacggggc tggtgctcag |
| 2041 | catcctcgtc cttgctgctg ctgctgctct gctgcattac |
| | gccagggccc gaaggaaacc |
| 2101 | aggaggactt tctgccactg gaacatctag tcacagtcct |
| | agtgagtgtc aggagccttc |
| 2161 | ctcgtccagg ccttccagga tagaccctca agagcccact |
| | cactctaaac cactagcccc |
| 2221 | aatggagctg gagccaatgt acagcaatgt aaatcctgga |
| | gatagcaacc cgatttattc |
| 2281 | ccagatctgg agcatccagc atacaaaaga aaactcagct |
| | aattgtccaa tgatgcatca |
| 2341 | agagcatgag gaacttacag tcctctattc agaactgaag |
| | aagacacacc cagacgactc |
| 2401 | tgcaggggag gctagcagca gaggcagggc ccatgaagaa |
| | gatgatgaag aaaactatga |
| 2461 | gaatgtacca cgtgtattac tggcctcaga ccactagccc |
| | cttacccaga gtggcccaca |
| 2521 | ggaaacagcc tgcaccattt ttttttctgt tctctccaac |
| | cacacatcat ccatctctcc |
| 2581 | agactctgcc tcctacgagg ctgggctgca gggtatgtga |
| | ggctgagcaa aaggtctgca |
| 2641 | aatctcccct gtgcctgatc tgtgtgttcc ccaggaagag |
| | agcaggcagc ctctgagcaa |
| 2701 | gcactgtgtt attttcacag tggagacacg tggcaaggca |
| | ggagggccct cagctcctag |
| 2761 | ggctgtcgaa tagaggagga gagagaaatg gtctagccag |
| | ggttacaagg gcacaatcat |
| 2821 | gaccatttga tccaagtgtg atcgaaagct gttaatgtgc |
| | tctctgtata aacaatttgc |
| 2881 | tccaaatatt ttgtttccct tttttgtgtg gctggtagtg |
| | gcattgctga tgttttggtg |
| 2941 | tatatgctgt atccttgcta ccatattggg aacagccaaa |
| | agaagttata gaacaagaat |
| 3001 | ttaaggtgac tctatctga |

An exemplary human SIGLEC8 amino acid sequence is set forth below (SEQ ID NO: 138; GenBank Accession No: Q9NYZ4.2, Version 2, incorporated herein by reference):

| | |
|---|---|
| 1 | mlllllllpl lwgtkgmegd rqygdgyllq vqelvtvqeg |
| | lcvhvpcsfs ypqdgwtdsd |
| 61 | pvhgywfrag drpyqdapva tnnpdrevqa etqgrfqllg |
| | diwsndcsls irdarkrdkg |
| 121 | syffrlergs mkwsyksqln yktkqlsvfv talthrpdil |
| | ilgtlesghs rnltcsvpwa |
| 181 | ckqgtppmis wigasvsspg pttarssvlt ltpkpqdhgt |
| | sltcqvtlpg tgvtttstvr |
| 241 | ldvsyppwnl tmtvfqgdat astalgngss lsvlegqslr |
| | lvcavnsnpp arlswtrgsl |
| 301 | tlcpsrssnp gllelprvhv rdegeftcra qnaqgsqhis |
| | lslslqnegt gtsrpvsqvt |
| 361 | laavggagat alaflsfcii fiivrscrkk sarpaagvgd |
| | tgmedakair gsasqgplte |
| 421 | swkdgnplkk pppavapssg eegelhyatl sfhkvkpqdp |
| | qgqeatdsey seikihkret |
| 481 | aetqaclrnh npsskevrg |

An exemplary human SIGLEC8 nucleic acid sequence is set forth below (SEQ ID NO: 139; GenBank Accession No: NM_014442.2, Version 2, incorporated herein by reference):

| | |
|---|---|
| 1 | agtttctgag agaagaaccc tgaggaacag acgttccctg |
| | gcggccctgg cgccttcaaa |
| 61 | cccagacatg ctgctgctgc tgctgctgct gcccctgctc |
| | tgggggacaa aggggatgga |
| 121 | gggagacaga caatatgggg atggttactt gctgcaagtg |
| | caggagctgg tgacggtgca |
| 181 | ggagggcctg tgtgtccatg tgccctgctc cttctcctac |
| | ccccaggatg gctggactga |
| 241 | ctctgaccca gttcatggct actggttccg ggcaggagac |
| | agaccatacc aagacgctcc |
| 301 | agtggccaca aacaacccag acagagaagt gcaggcagag |
| | acccagggcc gattccaact |
| 361 | ccttggggac atttggagca acgactgctc cctgagcatc |
| | agagacgcca ggaagaggga |
| 421 | taaggggtca tatttctttc ggctagagag aggaagcatg |
| | aaatggagtt acaaatcaca |
| 481 | gttgaattac aaaactaagc agctgtctgt gtttgtgaca |
| | gccctgaccc ataggcctga |
| 541 | catcctcatc ctagggaccc tagagtctgg ccactccagg |
| | aacctgacct gctctgtgcc |
| 601 | ctgggcctgt aagcagggga caccccccat gatctcctgg |
| | attggggcct ccgtgtcctc |
| 661 | cccgggcccc actactgccc gctcctcagt gctcaccctt |
| | accccaaagc cccaggacca |
| 721 | cggcaccagc ctcacctgtc aggtgacctt gcctgggaca |
| | ggtgtgacca cgaccagtac |
| 781 | cgtccgcctc gatgtgtcct accctccttg gaacttgacc |
| | atgactgtct tccaaggaga |
| 841 | tgccacagca tccacagccc tgggaaatgg ctcatctctt |
| | tcagtccttg agggccagtc |
| 901 | tctgcgcctg gtctgtgctg tcaacagcaa tccccctgcc |
| | aggctgagct ggacccgggg |
| 961 | gagcctgacc ctgtgcccct cacggtcctc aaaccctggg |
| | ctgctggagc tgcctcgagt |
| 1021 | gcacgtgagg gatgaagggg aattcacctg ccgagctcag |
| | aacgctcagg gctcccagca |
| 1081 | catttccctg agcctctccc tgcagaatga gggcacaggc |
| | acctcaagac ctgtatcaca |
| 1141 | agtgacactg gcagcagtcg ggggagctgg agccacagcc |
| | ctggccttcc tgtccttctg |
| 1201 | catcatcttc atcatagtga ggtcctgcag gaagaaatcg |
| | gcaaggccag cagcgggcgt |
| 1261 | gggggataca ggcatggaag atgcaaaggc catcaggggc |
| | tcggcctctc agggacccct |
| 1321 | gactgaatcc tggaaagatg gcaaccccct gaagaagcct |
| | cccccagctg ttgccccctc |
| 1381 | gtcaggggag gaaggagagc tccattatgc aaccctcagc |
| | ttccataaag tgaagcctca |
| 1441 | ggacccgcag ggacaggagg ccactgacag tgaatactcg |
| | gagatcaaga tccacaagcg |
| 1501 | agaaactgca gagactcagg cctgtttgag gaatcacaac |
| | ccctccagca aagaagtcag |
| 1561 | aggctgattc tcatagaaca agaaccctct agagccccat |
| | gctatgcagt aggtcaccag |
| 1621 | ggctccctcc tcctgtctaa ccaaaacttg gaccaatgtc |
| | tcccctttcc ccggctacca |
| 1681 | gggacccatc cctgcctcta gcttctacta cccaccattc |
| | tcctctcgac ctctctgagg |
| 1741 | ttgactattt tagattccac atagagatga ggtcatgtgg |
| | tacttgcctc tctgtgtgtg |
| 1801 | gctcatttta cacaaaaaaa tatcccctag gttcatccat |
| | gttctctcaa atgacagaat |
| 1861 | caagcactga atattttttt ttctttgaga gatggagttt |
| | cgctctgttg cccaggctgg |
| 1921 | agtgcagtgg ttcaatctct gctcactgca acctccacct |
| | cctgggttca aacgattctc |
| 1981 | ctgcctcagc ttcccaagta gctggtacta caggcgtgtg |
| | tcaccacgcc cagctaattt |
| 2041 | ttgtattttt tagtagagac ggggtttcac tataagtggg |
| | ccaggctagt ctcaaactcc |
| 2101 | tgacctcaag tgatctgcct gccttggcct cccaaagtgc |
| | tgggatttca ggcatgagcc |
| 2161 | accgcaccca gcttgcattg aatattttca aggagctaaa |
| | agaagatttt aaatggtctc |
| 2221 | acaaaaacag ataaatattt gcacagatgg gtgtgctaat |
| | cattgtgcct tgatggttcc |
| 2281 | acgatgtatc cgggtgtgga aatctcactg ggtctctctc |
| | aaggccactc ggctactcag |
| 2341 | gacagggctg gaatttaaag cctgtccgat tctgaggtct |
| | cttctctcat ctagcactga |
| 2401 | gtcaagcaat cagcaggctg ggcacccctt agccataagt |
| | tttcaggaaa taaattcctt |
| 2461 | gagggcattg acttttacaa aagagggagc agcaatggcc |
| | tagagtctca ggaacaagac |
| 2521 | aggtgcactg aggagatgaa ggccgggacc ccctgcccaa |
| | cctgtatggc gggtctgtac |
| 2581 | ttattttgtt tacccccaat ttaaaacgtt tttttttatt |
| | gcaggttgtt tgtttgatat |
| 2641 | ggtttggctg tgtccccacc caaatcttat ctagaattgt |
| | aatcagaatt ataatcccca |
| 2701 | tgtgttgggg gagggacctg gtgggaggtg ataggatcat |
| | gggggtggtt cccccatgct |
| 2761 | gttctgatag tgagtgagtt atcacgagat ctgatggttt |
| | tgtaagtggt ggtttcccct |
| 2821 | gctcttctct cttgcctgcc accatgtaag atgtgcctgg |
| | ttccccttcc gccatgattg |
| 2881 | taagtttcct gaggcctccc ccgccatgtg gaactgtgag |
| | tcaattacac ctctttcatt |
| 2941 | tataaattaa aaaaaaaaaa aaaaaaa |

An exemplary human FAIM3/TOSO amino acid sequence is set forth below (SEQ ID NO: 140; GenBank Accession No: 060667.1, Version 1, incorporated herein by reference):

| | |
|---|---|
| 1 | mdfwlwplyf lpvsgalril pevkvegelg gsvtikcplp |
| | emhvriylcr emagsgtcgt |
| 61 | vvsttnfika eykgrvtlkq yprknlflve vtqltesdsg |
| | vyacgagmnt drgktqkvtl |
| 121 | nvhseyepsw eeqpmpetpk wfhlpylfqm payassskfv |
| | trvttpaqrg kvppvhhssp |
| 181 | ttqithrprv srassvagdk prtflpstta skisalegll |
| | kpqtpsynhh trlhrqrald |
| 241 | ygsqsgregq gfhiliptil glfllallgl vvkraverrk |
| | alsrrarrla vrmralessq |
| 301 | rprgsprprs qnniysacpr rargadaagt geapvpgpga |
| | plppaplqvs espwlhapsl |
| 361 | ktsceyvsly hqpaammeds dsddyinvpa |

An exemplary human FAIM3/TOSO nucleic acid sequence is set forth below (SEQ ID NO: 141; GenBank Accession No: BC006401.2, Version 2, incorporated herein by reference):

| | |
|---|---|
| 1 | atttcctcat cgtcaagctt tgttcctcgt gggggctaga |
| | aatctctttc cagttccaga |
| 61 | ttgtgaaggg ttcctgagta agcagcgtgt ctccatcccc |
| | ctctctaggg gctcttggat |
| 121 | ggaccttgca ctctagaagg gacaatggac ttctggcttt |
| | ggccacttta cttcctgcca |
| 181 | gtatcggggg ccctgaggat cctcccagaa gtaaaggtag |
| | agggggagct gggcggatca |
| 241 | gttaccatca agtgcccact tcctgaaatg catgtgagga |
| | tatatctgtg ccgggagatg |
| 301 | gctggatctg gaacatgtgg taccgtggta tccaccacca |
| | acttcatcaa ggcagaatac |
| 361 | aagggccgag ttactctgaa gcaataccca cgcaagaatc |
| | tgttcctagt ggaggtaaca |
| 421 | cagctgacag aaagtgacag cggagtctat gcctgcggag |
| | cgggcatgaa cacagaccgg |
| 481 | ggaaagaccc agaaagtcac cctgaatgtc cacagtgaat |
| | acgagccatc atgggaagag |
| 541 | cagccaatgc ctgagactcc aaaatggttt catctgccct |
| | atttgttcca gatgcctgca |
| 601 | tatgccagtt cttccaaatt cgtaaccaga gttaccacac |
| | cagctcaaag gggcaaggtc |
| 661 | cctccagttc accactcctc ccccaccacc caaatcaccc |
| | accgccctcg agtgtccaga |
| 721 | gcatcttcag tagcaggtga caagccccga accttcctgc |
| | catccactac agcctcaaaa |
| 781 | atctcagctc tggaggggct gctcaagccc cagacgccca |
| | gctacaacca ccacaccagg |
| 841 | ctgcacaggc agagagcact ggactatggc tcacagtctg |
| | ggagggaagg ccaaggattt |
| 901 | cacatcctga tcccgaccat cctgggcctt ttcctgctgg |
| | cacttctggg gctggtggtg |
| 961 | aaaagggccg ttgaaaggag gaaagccctc tccaggcggg |
| | cccgccgact ggccgtgagg |
| 1021 | atgcgcgccc tggagagctc ccagaggccc cgcgggtcgc |
| | cgcgaccgcg ctcccaaaac |
| 1081 | aacatctaca gcgcctgccc gcggcgcgct cgtggagcgg |
| | acgctgcagg cacaggggag |
| 1141 | gcccccgttc ccggccccgg agcgccgttg ccccccgccc |
| | cgctgcaggt gtctgaatct |
| 1201 | ccctggctcc atgccccatc tctgaagacc agctgtgaat |
| | acgtgagcct ctaccaccag |
| 1261 | cctgccgcca tgatggagga cagtgattca gatgactaca |
| | tcaatgttcc tgcctgacaa |
| 1321 | ctccccagct atcccccaac cccaggctcg gactgtggtg |
| | ccaaggagtc tcatctatct |
| 1381 | gctgatgtcc aatacctgct tcatgtgttc tcagagccct |
| | catcacttcc catgccccat |
| 1441 | ctcgactccc atccccatct atctgtgccc tgagcatggc |
| | tctgccccca ggtcgtcttg |
| 1501 | cacaccttgg cagccccctg tagttgacag gtaagctgta |
| | ggcatgtaga gcaattgtcc |
| 1561 | caatgccact tgcttccttt ccaagccgtc gaacagactg |
| | tgggatttgc agagtgtttc |
| 1621 | ttccatgtct ttgaccacag ggttgttgct gcccaggctc |
| | tagatcacat ggcatcaggc |
| 1681 | tggggcagag gcatagctat tgtctcgggc atccttccca |
| | gggttgggtc ttacacaaat |
| 1741 | agaaggctct tgctctgagt tatgtgacat gcctcagccc |
| | catggactaa gcaggggtct |
| 1801 | ggtataaaaa cactcctgga aacgcctttg ccctgatcca |
| | aatgttagca cttgctagtg |
| 1861 | aacgtctact tatctcaagt tctatgctaa aggcaattta |
| | tcttgatgtg atgataaacc |
| 1921 | aaacttatta gcaagatatg catatatatc aaaaaaaaaa |
| | aaaaaaaa |

An exemplary human MAGEA2B amino acid sequence is set forth below (SEQ ID NO: 142; GenBank Accession No: AAI12161.1, Version 1, incorporated herein by reference):

| | |
|---|---|
| 1 | mdfwlwplyf lpvsgalril pevkvegelg gsvtikcplp |
| | emhvriylcr emagsgtcgt |
| 61 | vvsttnfika eykgrvtlkq yprknlflve vtqltesdsg |
| | vyacgagmnt drgktqkvtl |
| 121 | nvhseyepsw eeqpmpetpk wfhlpylfqm payassskfv |
| | trvttpaqrg kvppvhhssp |
| 181 | ttqithrprv srassvagdk prtflpstta skisalegll |
| | kpqtpsynhh trlhrqrald |
| 241 | ygsqsgregq gfhiliptil glfllallgl vvkraverrk |
| | alsrrarrla vrmralessq |
| 301 | rprgsprprs qnniysacpr rargadaagt geapvpgpga |
| | plppaplqvs espwlhapsl |
| 361 | ktsceyvsly hqpaammeds dsddyinvpa |

An exemplary human MAGEA2B nucleic acid sequence is set forth below (SEQ ID NO: 143; GenBank Accession No: NM_001321400.1, Version 1, incorporated herein by reference):

| | |
|---|---|
| 1 | ttgcgcattg gaggtcagag gacagcgaga ttctcgccct |
| | gagcaacggc ctgacgtcgg |
| 61 | cggagggaag caggcgcagg ctccgtgagg aggcaagaat |
| | ccgggctttg cccctgcaat |
| 121 | caacccacgg aagctccggg aatggcggcc aagcacgcgg |
| | atcctgacgt tcacatatct |
| 181 | cagggagttg atgaccttgt tttcagaagg tgactcaggt |
| | caacacaggg gcccccatct |
| 241 | ggtcgacaga tgcagtggtt ctaggatctg ccaagcatcc |
| | aggtggagag cctgaggaat |
| 301 | caggagctcc aggaaccagg cagtgaggcc ttggtctgag |
| | tcagtgtcct caggtcacag |
| 361 | agcagagggg acgcagacag tgccaacact gaaggttctg |
| | agggggacag gctgacaagt |
| 421 | aggacccgag gcactggagg agcattgaag gagaagatct |
| | gcctgtgggt cttcattgcc |
| 481 | cagctcctgc ccgcactcct gcctgctgcc ctgaccagag |
| | tcatcatgcc tcttgagcag |
| 541 | aggagtcagc actgcaagcc tgaagaaggc cttgaggccc |
| | gaggagaggc cctgggcctg |
| 601 | gtgggtgcgc aggctcctgc tactgaggag cagcagaccg |
| | cttcttcctc ttctactcta |
| 661 | gtggaagtta ccctggggga ggtgcctgct gccgactcac |
| | cgagtcctcc ccacagtcct |
| 721 | cagggagcct ccagcttctc gactaccatc aactacactc |
| | tttggagaca atccgatgag |
| 781 | ggctccagca accaagaaga ggaggggcca agaatgtttc |
| | ccgacctgga gtccgagttc |
| 841 | caagcagcaa tcagtaggaa gatggttgag ttggttcatt |
| | ttctgctcct caagtatcga |
| 901 | gccagggagc cggtcacaaa ggcagaaatg ctggagagtg |
| | tcctcagaaa ttgccaggac |
| 961 | ttctttcccg tgatcttcag caaagcctcc gagtacttgc |
| | agctggtctt tggcatcgag |
| 1021 | gtggtggaag tggtccccat cagccacttg tacatccttg |
| | tcacctgcct gggcctctcc |
| 1081 | tacgatggcc tgctgggcga caatcaggtc atgcccaaga |
| | caggcctcct gataatcgtc |
| 1141 | ctggccataa tcgcaataga gggcgactgt gcccctgagg |
| | agaaaatctg ggaggagctg |
| 1201 | agtatgttgg aggtgtttga ggggagggag gacagtgtct |
| | tcgcacatcc caggaagctg |
| 1261 | ctcatgcaag atctggtgca ggaaaactac ctggagtacc |
| | ggcaggtgcc cggcagtgat |
| 1321 | cctgcatgct acgagttcct gtggggtcca agggccctca |
| | ttgaaaccag ctatgtgaaa |
| 1381 | gtcctgcacc atacactaaa gatcggtgga gaacctcaca |
| | tttcctaccc acccctgcat |
| 1441 | gaacgggctt tgagagaggg agaagagtga gtctcagcac |
| | atgttgcagc cagggccagt |
| 1501 | gggagggggt ctgggccagt gcaccttcca gggccccatc |
| | cattagcttc cactgcctcg |
| 1561 | tgtgatatga ggcccattcc tgcctctttg aagagagcag |
| | tcagcattct tagcagtgag |
| 1621 | tttctgttct gttggatgac tttgagattt atctttgttt |
| | cctgttggaa ttgttcaaat |
| 1681 | gttcctttta acaaatggtt ggatgaactt cagcatccaa |
| | gtttatgaat gacagtagtc |
| 1741 | acacatagtg ctgtttatat agtttagggg taagagtcct |
| | gttttttatt cagattggga |
| 1801 | aatccattcc attttgtgag ttgtcacata ataacagcag |
| | tggaatatgt atttgcctat |
| 1861 | attgtgaacg aattagcagt aaaatacatg atacaaggaa |
| | ctcaaaagat agttaattct |
| 1921 | tgccttatac ctcagtctat tatgtaaaat taaaaatatg |
| | tgtatgtttt tgcttctttg |
| 1981 | agaatgcaaa agaaattaaa tctgaataaa taattcttcc |
| | tgttca |

An exemplary human MKRN9P amino acid sequence is set for the below (SEQ ID NO: 144; GenBank Accession No: Q6NVV0.1, Version 1, incorporated herein by reference):

| | |
|---|---|
| 1 | mllaavgdde ltdsedesdl fheeledfyd ldl |

An exemplary human MKRN9P nucleic acid sequence is set forth below (SEQ ID NO: 145; GenBank Accession No: NR_033410.1, Version 1, incorporated herein by reference):

| | |
|---|---|
| 1 | gacgtggagc aggcagggac tgagcgggtg cctcagtgtc |
| | ctacccctcc cttcgccggg |
| 61 | cctctcagtc ctctcccagc agctggaccg gaactatgtg |
| | atcccggaag ttccggggcc |
| 121 | ttttctgtgt gggataaaca gtaatggcgg aggctgcagc |
| | tctcagacca acagccacaa |
| 181 | catcaggggc ggcagtggcg gcagcggcgg cggcggcagc |
| | agcggcctcc cctaccagga |
| 241 | tccccacagt caccattctg tccccagggg gcaggcgagg |
| | gggccgcggc agcgatggca |
| 301 | gcggcggcag ctggactaaa caggtcacct gcaggtattt |
| | tatgcatggg gtttataagg |
| 361 | aaggagataa ctgttgcaag ttgcatgacc tctctgacag |
| | tccgtataat gtagtgtaca |
| 421 | agagtttgca gccagggtac tgtatttatg gagactgctg |
| | cagatatgaa catagcaagc |
| 481 | cattgaaaca gaaagaagca actgctacag agctaactac |
| | aaagtcatcc cttgctgctt |
| 541 | cctcaagtct ctcatggata gtcggaccac ttgttgaaat |
| | gaatacgggt gaagctgagt |
| 601 | caagaaattc aaactttgta actgtaggag caggttcaga |
| | gggctgggtg aatgctgttg |
| 661 | agtttgttcc tgggcaaccc tactgtggcc gcactgcgcc |
| | ttcctgcact gaagcacccc |
| 721 | tgcagggctc agtgaccaag gaagaatcag agaaagagca |
| | aactgccgtg gaaacaaaga |
| 781 | agcagctgtg cccctatgct gcagtgggag agtgccgata |
| | tcgggagaac ttgtgtgtat |
| 841 | atggagattc gtgtgacatg tgtggctgca ggtcctgcat |
| | ctgatggatg ctgcccagag |
| 901 | atcacagcat ataaaatcat gctttgaggc ccatgagaaa |
| | gacagggagc tctcatttgc |
| 961 | tgtgcagcgc agcgaggaaa tggtgtgtga gagctgcatg |
| | gagatgatct atgagaaagc |
| 1021 | caaccccaga cagcaccgct tcgggatcct ctccaactgc |
| | aaccacacct actgtcttaa |
| 1081 | gtgcattcgc aagtggagga gtgctaagca gtttgagagc |
| | aagatcataa agtcctgccc |
| 1141 | agaatgtcgg atcacatcta actttgtcgt tccaagtgag |
| | tactgtgtgg aggagaaaga |
| 1201 | agagaagcag aaactcattc tgaaatacaa ggaggcaatg |
| | agcaacaagg cgtgcaggta |
| 1261 | ttttgatgaa ggacatggga gctgcccgtt tggagggaac |
| | ggtttttata agcatgtgta |
| 1321 | ccctgatggc cgtagagagg agccacagag acagaaggtg |
| | ggaacatcaa gcagataccc |
| 1381 | ggcccaatga aggaaccact tctgggaact cattgaggaa |
| | agagagaaca gcagcccctt |
| 1441 | tgacaacgat gaagaagagg ttgtcacctt tgagctgggt |
| | gagttgttgc ttatgctttt |
| 1501 | ggctgcagtt ggggacgatg aactaacaga ctctgaagat |
| | gagagcgact tgtttcatga |
| 1561 | ggagctggaa gatttttatg acttggatct atagcaacct |
| | tgtgtggcgt gtgaactggt |
| 1621 | ctgctgaccc cagacagcag ctgttccctg tggtggtgtg |
| | gcagtgcctg tgttctctcc |
| 1681 | taggcaggcc tgtcaactcc aggtgctggc ataagaattt |
| | ttacccaggg cctgtctttt |
| 1741 | caacccctca cctttcccca aggagtgtgt tgttttccct |
| | cttgaaaaaa agttagaaaa |
| 1801 | ataaatctta aagttagttt tctgaaa |

An exemplary human MAGEC1 amino acid sequence is set forth below (SEQ ID NO: 146; GenBank Accession No: 060732.3, Version 3, incorporated herein by reference):

| | |
|---|---|
| 1 | mgdkdmptag mpsllqssse spqscpeged sqsplqipqs |
| | spesddtlyp lqspqsrseg |
| 61 | edssdplqrp pegkdsqspl qipqsspegd dtqsplqnsq |
| | sspegkdsls pleisqsppe |
| 121 | gedvqsplqn passffssal lsifqsspes tqspfegfpq |
| | svlqipvsaa ssstlvsifq |
| 181 | sspestqspf egfpqsplqi pvsrsfsstl lsifqssper |
| | tqstfegfaq splqipvsps |
| 241 | ssstllslfq sfsertqstf egfaqsslqi pvspsfsstl |
| | vslfqssper tqstfegfpq |
| 301 | splqipvsss ssstllslfq ssperthstf egfpqsllqi |
| | pmtssfsstl lsifqsspes |
| 361 | aqstfegfpq splqipgsps fsstllslfq ssperthstf |
| | egfpqsplqi pmtssfsstl |
| 421 | lsilqsspes aqsafegfpq splqipvsss fsytllslfq |
| | ssperthstf egfpqsplqi |
| 481 | pvssssssst llslfqsspe ctqstfegfp qsplqipqsp |
| | pegenthspl qivpslpewe |
| 541 | dslsphyfpq sppqgedsls phyfpqsppq gedslsphyf |
| | pqspqgedsl sphyfpqspp |
| 601 | qgedsmsply fpqsplqgee fqsslqspvs icssstpssl |
| | pqsfpessqs ppegpvqspl |
| 661 | hspqsppegm hsqsplqspe sapegedsls plqipqsple |
| | gedslsslhf pqsppeweds |
| 721 | lsplhfpqfp pqgedfqssl qspvsicsss tslslpqsfp |
| | espqsppegp aqsplqrpvs |
| 781 | sffsytlasl lqsshespqs ppegpaqspl qspvssfpss |
| | tssslsqssp vssfpsstss |
| 841 | slsksspesp lqspvisfss stslspfsee ssspvdeyts |
| | ssdtllesds ltdseslies |
| 901 | eplftytlde kvdelarfll lkyqvkqpit kaemltnvis |
| | rytgyfpvif rkarefieil |
| 961 | fgislrevdp ddsyvfvntl dltsegclsd eqgmsqnrll |
| | ililsiifik gtyaseeviw |
| 1021 | dvlsgigvra grehfafgep relltkvwvq ehyleyrevp |
| | nssppryefl wgprahsevi |
| 1081 | krkvveflam lkntvpitfp ssykdalkdv eeraqaiidt |
| | tddstatesa sssvmspsfs |
| 1141 | se |

An exemplary human MAGEC1 nucleic acid sequence is set forth below (SEQ ID NO: 147; GenBank Accession No: NM_005462.4, Version 4, incorporated herein by reference):

| | |
|---|---|
| 1 | gctttgccgg atgtgctttc ccggcggcca tcttgggagt |
| | ctgaaggacc tgaggcattt |
| 61 | tgtgacgagg atcgtctcag gtcagcggag ggaggagact |
| | tatagaccta tccagtcttc |
| 121 | aaggtgctcc agaaagcagg agttgaagac ctgggtgtga |
| | gggacacata catcctaaaa |
| 181 | gcaccacagc agaggaggcc caggcagtgc caggagtcaa |
| | ggttcccaga agacaaaccc |
| 241 | cctaggaaga caggcgacct gtgaggccct agagcaccac |
| | cttaagagaa gaagagctgt |
| 301 | aagccggcct ttgtcagagc catcatgggg gacaaggata |
| | tgcctactgc tgggatgccg |
| 361 | agtcttctcc agagttcctc tgagagtcct cagagttgtc |
| | ctgaggggga ggactcccag |
| 421 | tctcctctcc agattcccca gagttctcct gagagcgacg |
| | acaccctgta tcctctccag |
| 481 | agtcctcaga gtcgttctga gggggaggac tcctcggatc |
| | ctctccagag acctcctgag |
| 541 | gggaaggact cccagtctcc tctccagatt ccccagagtt |
| | ctcctgaggg cgacgacacc |
| 601 | cagtctcctc tccagaattc tcagagttct cctgagggga |
| | aggactccct gtctcctcta |
| 661 | gagatttctc agagccctcc tgagggtgag gatgtccagt |
| | ctcctctgca gaatcctgcg |
| 721 | agttccttct tctcctctgc tttattgagt attttccaga |
| | gttcccctga gagtactcaa |
| 781 | agtccttttg agggttttcc ccagtctgtt ctccagattc |
| | ctgtgagcgc cgcctcctcc |
| 841 | tccactttag tgagtatttt ccagagttcc cctgagagta |
| | ctcaaagtcc ttttgagggt |
| 901 | tttccccagt ctccactcca gattcctgtg agccgctcct |
| | tctcctccac tttattgagt |
| 961 | attttccaga gttcccctga gagaactcag agtacttttg |
| | agggttttgc ccagtctcct |
| 1021 | ctccagattc ctgtgagccc ctcctcctcc tccactttac |
| | tgagtctttt ccagagtttc |
| 1081 | tctgagagaa ctcagagtac ttttgagggt tttgcccagt |
| | cttctctcca gattcctgtg |
| 1141 | agcccctcct tctcctccac tttagtgagt cttttccaga |
| | gttcccctga gagaactcag |
| 1201 | agtacttttg agggttttcc ccagtctcct ctccagattc |
| | ctgtgagctc ctcctcctcc |
| 1261 | tccactttat tgagtctttt ccagagttcc cctgagagaa |
| | ctcacagtac ttttgagggt |
| 1321 | tttccccagt ctcttctcca gattcctatg acctcctcct |
| | tctcctctac tttattgagt |
| 1381 | attttccaga gttctcctga gagtgctcaa agtacttttg |
| | agggttttcc ccagtctcct |
| 1441 | ctccagattc ctgggagccc ctccttctcc tccactttac |
| | tgagtctttt ccagagttcc |
| 1501 | cctgagagaa ctcacagtac ttttgagggt tttccccagt |
| | ctcctctcca gattcctatg |
| 1561 | acctcctcct tctcctctac tttattgagt attttacaga |
| | gttctcctga gagtgctcaa |
| 1621 | agtgcttttg agggttttcc ccagtctcct ctccagattc |
| | ctgtgagctc ctctttctcc |
| 1681 | tacactttat tgagtctttt ccagagttcc cctgagagaa |
| | ctcacagtac ttttgagggt |
| 1741 | tttccccagt ctcctctcca gattcctgtg agctcctcct |
| | cctcctcctc cactttattg |
| 1801 | agtcttttcc agagttcccc tgagtgtact caaagtactt |
| | ttgagggttt tccccagtct |
| 1861 | cctctccaga ttcctcagag tcctcctgaa ggggagaata |
| | cccattctcc tctccagatt |
| 1921 | gttccaagtc ttcctgagtg ggaggactcc ctgtctcctc |
| | actactttcc tcagagccct |
| 1981 | cctcaggggg aggactccct atctcctcac tactttcctc |
| | agagccctcc tcagggggag |
| 2041 | gactccctgt ctcctcacta ctttcctcag agccctcagg |
| | gggaggactc cctgtctcct |
| 2101 | cactactttc ctcagagccc tcctcagggg gaggactcca |
| | tgtctcctct ctactttcct |
| 2161 | cagagtcctc ttcaggggga ggaattccag tcttctctcc |
| | agagccctgt gagcatctgc |
| 2221 | tcctcctcca ctccatccag tcttccccag agtttccctg |
| | agagttctca gagtcctcct |
| 2281 | gaggggcctg tccagtctcc tctccatagt cctcagagcc |
| | ctcctgaggg gatgcactcc |
| 2341 | caatctcctc tccagagtcc tgagagtgct cctgaggggg |
| | aggattccct gtctcctctc |
| 2401 | caaattcctc agagtcctct tgagggagag gactccctgt |
| | cttctctcca ttttcctcag |
| 2461 | agtcctcctg agtgggagga ctccctctct cctctccact |
| | ttcctcagtt tcctcctcag |
| 2521 | ggggaggact tccagtcttc tctccagagt cctgtgagta |
| | tctgctcctc ctccacttct |
| 2581 | ttgagtcttc cccagagttt ccctgagagt cctcagagtc |
| | ctcctgaggg gcctgctcag |
| 2641 | tctcctctcc agagacctgt cagctccttc ttctcctaca |
| | ctttagcgag tcttctccaa |
| 2701 | agttcccatg agagtcctca gagtcctcct gaggggcctg |
| | cccagtctcc tctccagagt |
| 2761 | cctgtgagct ccttcccctc ctccacttca tcgagtcttt |
| | cccagagttc tcctgtgagc |
| 2821 | tccttcccct cctccacttc atcgagtctt tccaagagtt |
| | cccctgagag tcctctccag |
| 2881 | agtcctgtga tctccttctc ctcctccact tcattgagcc |
| | cattcagtga agagtccagc |
| 2941 | agcccagtag atgaatatac aagttcctca gacaccttgc |
| | tagagagtga ttccttgaca |
| 3001 | gacagcgagt ccttgataga gagcgagccc ttgttcactt |
| | atacactgga tgaaaaggtg |
| 3061 | gacgagttgg cgcggtttct tctcctcaaa tatcaagtga |
| | agcagcctat cacaaaggca |
| 3121 | gagatgctga cgaatgtcat cagcaggtac acgggctact |
| | ttcctgtgat cttcaggaaa |
| 3181 | gcccgtgagt tcatagagat actttttggc atttccctga |
| | gagaagtgga ccctgatgac |
| 3241 | tcctatgtct ttgtaaacac attagacctc acctctgagg |
| | ggtgtctgag tgatgagcag |
| 3301 | ggcatgtccc agaaccgcct cctgattctt attctgagta |
| | tcatcttcat aaagggcacc |
| 3361 | tatgcctctg aggaggtcat ctgggatgtg ctgagtggaa |
| | taggggtgcg tgctgggagg |
| 3421 | gagcactttg cctttgggga gcccagggag ctcctcacta |
| | aagtttgggt gcaggaacat |
| 3481 | tacctagagt accgggaggt gcccaactct tctcctcctc |
| | gttacgaatt cctgtggggt |
| 3541 | ccaagagctc attcagaagt cattaagagg aaagtagtag |
| | agtttttggc catgctaaag |
| 3601 | aataccgtcc ctattacctt tccatcctct tacaaggatg |
| | ctttgaaaga tgtggaagag |
| 3661 | agagcccagg ccataattga caccacagat gattcgactg |
| | ccacagaaag tgcaagctcc |
| 3721 | agtgtcatgt cccccagctt ctcttctgag tgaagtctag |
| | ggcagattct tccctctgag |
| 3781 | tttgaagggg gcagtcgagt ttctacgtgg tggagggcct |
| | ggttgaggct ggagagaaca |
| 3841 | cagtgctatt tgcatttctg ttccatatgg gtagttatgg |
| | ggtttacctg ttttactttt |
| 3901 | gggtattttt caaatgcttt tcctattaat aacaggttta |
| | aatagcttca gaatcctagt |
| 3961 | ttatgcacat gagtcgcaca tgtattgctg tttttctggt |
| | ttaagagtaa cagtttgata |
| 4021 | ttttgtaaaa acaaaaacac acccaaacac accacattgg |
| | gaaaaccttc tgcctcattt |
| 4081 | tgtgatgtgt cacaggttaa tgtggtgtta ctgtaggaat |
| | tttcttgaaa ctgtgaagga |
| 4141 | actctgcagt taaatagtgg aataaagtaa aggattgtta |
| | atgtttgcat ttcctcaggt |
| 4201 | cctttagtct gttgttcttg aaaactaaag atacatacct |
| | ggtttgcttg gcttacgtaa |
| 4261 | gaaagtagaa gaaagtaaac tgtaataaat aaaagtgtca |
| | gtgactcatt tatttgatga |
| 4321 | aaaaaaaaaa aaaaaaa |

An exemplary human PSG11 amino acid sequence is set forth below (SEQ ID NO: 148; GenBank Accession No: AAA60203.1, Version 1, incorporated herein by reference):

| | |
|---|---|
| 1 | mgpfpapsct qritwkglll tasllnfwnp pttaevtiea |
| | qppkvsegkd vlllvhnlpq |
| 61 | nlpgyfwykg emtdlyhyii syivdgkiii ygpaysgret |
| | vysnaslliq nvtrkdagty |
| 121 | tlhiikrgde treeirhftf tlyletpkpy isssnlnpre |
| | ameavrlicd petldasylw |
| 181 | wmngqslpvt hrlqlsktnr tlylfgvtky iagpyeceir |
| | npvsairsdp vtlnllpklp |
| 241 | ipyitinnln prenkdvlaf tcepksenyt yiwwlngqsl |
| | pvspgvkrpi enrililpsv |
| 301 | trnetgpyqc eirdrygglr snpvilnvly gpdlpriyps |
| | ftyyrsgenl dlscftesnp |
| 361 | paeyfwting kfqqsgqklf ipqitrnhsg lyacsvhnsa |
| | tgkeisksmt vkvsgpchgd |
| 421 | ltesqs |

An exemplary human PSG11 nucleic acid sequence is set forth below (SEQ ID NO: 149; GenBank Accession No: M58591.1, Version 1, incorporated herein by reference):

| | |
|---|---|
| 1 | cagccgtgct cagacagctt ctggatccta ggctcatctc |
| | cacagaggag aacacgcagg |
| 61 | cagcagagac catggggccc ttcccagccc cttcctgcac |
| | acagcgcatc acctggaagg |
| 121 | ggctcctgct cacagcatca cttttaaact tctggaaccc |
| | gcccaccact gccgaagtca |
| 181 | cgattgaagc ccagccaccc aaagtttctg aggggaagga |
| | tgttcttcta cttgtccaca |
| 241 | atttgcccca gaatcttcct ggctacttct ggtacaaagg |
| | ggaaatgacg gacctctacc |
| 301 | attacattat atcgtatata gttgatggta aaataattat |
| | atatgggcct gcatacagtg |
| 361 | gaagagaaac agtatattcc aacgcatccc tgctgatcca |
| | gaatgtcacc cggaaggatg |
| 421 | caggaaccta caccttacac atcataaagc gaggtgatga |
| | gactagagaa gaaattcgac |
| 481 | atttcacctt caccttatac ttggagactc ccaagcccta |
| | catctccagc agcaacttaa |
| 541 | accccaggga ggccatggag gctgtgcgct taatctgtga |
| | tcctgagact ctggacgcaa |
| 601 | gctacctatg gtggatgaat ggtcagagcc tccctgtgac |
| | tcacaggttg cagctgtcca |
| 661 | aaaccaacag gaccctctat ctatttggtg tcacaaagta |
| | tattgcagga ccctatgaat |
| 721 | gtgaaatacg gaacccagtg agtgccattc gcagtgaccc |
| | agtcaccctg aatctcctcc |
| 781 | cgaagctgcc catcccctac atcaccatca acaacttaaa |
| | ccccagggag aataaggatg |
| 841 | tcttagcctt cacctgtgaa cctaagagtg agaactacac |
| | ctacatttgg tggctaaacg |
| 901 | gtcagagcct ccccgtcagt cccggggtaa agcgacccat |
| | tgaaaacagg atactcattc |
| 961 | tacccagtgt cacgagaaat gaaacaggac cctatcaatg |
| | tgaaatacgg gaccgatatg |
| 1021 | gtggcctccg cagtaaccca gtcatcctaa atgtcctcta |
| | tggtccagac ctccccagaa |
| 1081 | tttacccttc attcacctat taccgttcag gagaaaacct |
| | cgacttgtcc tgcttcacgg |
| 1141 | aatctaaccc accggcagag tatttttgga caattaatgg |
| | gaagtttcag caatcaggac |
| 1201 | aaaagctctt tatcccccaa attactagaa atcatagcgg |
| | gctctatgct tgctctgttc |
| 1261 | ataactcagc cactggcaag gaaatctcca aatccatgac |
| | agtcaaagtc tctggtccct |
| 1321 | gccatggaga cctgacagag tctcagtcat gactgcaaca |
| | actgagacac tgagaaaaag |
| 1381 | aacaggctga taccttcatg aaattcaaga caaagaagaa |
| | aaaaactcaa tgttattgga |
| 1441 | ctaaataatc aaaaggataa tgttttcata attttttatt |
| | ggaaaatgtg ctgattcttt |
| 1501 | gaatgtttta ttctccagat ttatgaactt tttttcttca |
| | gcaattggta aagtatactt |
| 1561 | ttgtaaacaa aaattgaaat atttgctttt gctgtctatc |
| | tgaatgcccc agaattgtga |
| 1621 | aactattcat gagtattcat aggtttatgg taataaagtt |
| | atttgcacat gttccgta |

An exemplary human TAC3 amino acid sequence is set forth below (SEQ ID NO: 150; GenBank Accession No: AAQ89042.1, Version 1, incorporated herein by reference):

| | |
|---|---|
| 1 | mrimllftai lafslaqsfg avckepqeev vpgggrskrd |
| | pdlyqllqrl fkshsslegl |
| 61 | lkalsqastd pkestspekr dmhdffvglm gkrsvqpegk |
| | tgpflpsvrv prplhpnqlg |
| 121 | stgksslgte eqrpl |

An exemplary human TAC3 nucleic acid sequence is set forth below (SEQ ID NO: 151; GenBank Accession No: AY358679.1, Version 1, incorporated herein by reference):

| | |
|---|---|
| 1 | agtgactgca gccttcctag atcccctcca ctcggtttct |
| | ctctttgcag gagcaccggc |
| 61 | agcaccagtg tgtgagggga gcaggcagcg gtcctagcca |
| | gttccttgat cctgccagac |
| 121 | cacccagccc ccggcacaga gctgctccac aggcaccatg |
| | aggatcatgc tgctattcac |
| 181 | agccatcctg gccttcagcc tagctcagag ctttggggct |
| | gtctgtaagg agccacagga |
| 241 | ggaggtggtt cctggcgggg gccgcagcaa gagggatcca |
| | gatctctacc agctgctcca |
| 301 | gagactcttc aaaagccact catctctgga gggattgctc |
| | aaagccctga gccaggctag |
| 361 | cacagatcct aaggaatcaa catctcccga gaaacgtgac |
| | atgcatgact tctttgtggg |
| 421 | acttatgggc aagaggagcg tccagccaga gggaaagaca |
| | ggacctttct taccttcagt |
| 481 | gagggttcct cggccccttc atcccaatca gcttggatcc |
| | acaggaaagt cttccctggg |
| 541 | aacagaggag cagagacctt tataagactc tcctacggat |
| | gtgaatcaag agaacgtccc |
| 601 | cagctttggc atcctcaagt atcccccgag agcagaatag |
| | gtactccact tccggactcc |
| 661 | tggactgcat taggaagacc tctttccctg tcccaatccc |
| | caggtgcgca cgctcctgtt |
| 721 | accctttctc ttccctgttc ttgtaacatt cttgtgcttt |
| | gactccttct ccatcttttc |
| 781 | tacctgaccc tggtgtggaa actgcatagt gaatatcccc |
| | aaccccaatg ggcattgact |
| 841 | gtagaatacc ctagagttcc tgtagtgtcc tacattaaaa |
| | atataatgtc tctctctatt |
| 901 | cctcaacaat aaaggatttt tgcatatgaa aaaaaaaaaa |
| | aaaaaaaaaa aaaaaaaaaa |
| 961 | aaaaaaaaaa aa |

An exemplary human PSG8 amino acid sequence is set forth below (SEQ ID NO: 152; GenBank Accession No: Q9UQ74.2, Version 2, incorporated herein by reference):

| | |
|---|---|
| 1 | mgllsappct qritwkglll tasllnfwnp pttaqvtiea |
| | qptkvsegkd vlllvhnlpq |
| 61 | nitgyiwykg qirdlyhyit syvvdgqiii ygpaysgret |
| | iysnaslliq nvtqedagsy |
| 121 | tlhiimggde nrgvtghftf tlyletpkps isssklnpre |
| | ameaysltcd petpdasylw |
| 181 | wmngqslpms hrlqlsetnr tlfllgvtky tagpyeceir |
| | npvsasrsdp ftlnllpklp |
| 241 | kpyitinnlk prenkdvlnf tcepksenyt yiwwlngqsl |
| | pvsprvkrpi enrililpsv |
| 301 | trnetgpyqc eirdqyggir sypvtlnvly gpdlpriyps |
| | ftyyrsgevl ylscsadsnp |
| 361 | paqyswting kfqlsgqklf ipqittkhsg lyacsvrnsa |
| | tgkessksmt vkvsgkripv |
| 421 | slaigi |

An exemplary human PSG8 nucleic acid sequence is set forth below (SEQ ID NO: 153; GenBank Accession No: AH007519.2, Version 2, incorporated herein by reference):

| | |
|---|---|
| 1 | aggactctgc ttgcccaacc tgcagggtat gtttctgact gtgtaggtct gtgtgtgtcc |
| 61 | ttctgtgtgt gtgtgtgtgt gtgtgtgtgt gtgtgtgtgt gtgtgtgtgt gtgtgtgtgt |
| 121 | ctgcacaaag tgtgtgttga ggtttggtga aagaatcact gctgaaaaaa ggcagaggcc |
| 181 | tccacaattc ccagggacct gaaacacaga caaaaggaaa aacaggaggg acaaggaggc |
| 241 | aggactgaga gaggagggga cagagaggtg tcctgggcct gaccccgccc atgaacctga |
| 301 | gaggtgctcc tgccccggga agaagctcag cgcagaagga ggcaggacag cactgctgag |
| 361 | agctgtgctc aggaagcttc tggatcctag gctcatctcc acagaggaga acacacagac |
| 421 | agcagagacc atggggctcc tctcagcccc tccctgcaca cagcgcatca cctggaaggg |
| 481 | gctcctgctc acaggtgagg agagaacctc ctgggagagg acaggaggag gaagcagagt |
| 541 | gactggatgg ggtctccttg agagtatggg gtactaaaaa atgaaagaag ccagcacttt |
| 601 | gggaggctga ggcaggtgga tcatgagatc aggagttcaa gatcagtctg gccaacacag |
| 661 | tgaagccctg tctctactaa aaatacaaaa atttaaccag atattgtggt gtgctcctct |
| 721 | aatcctagct actcgggagg ctgaggcaga agaatcacgt gaacccagga ggcagaagtt |
| 781 | gcagctagct gagatagtgc catcgcacgc cagcctggga gacagtatga gactccatct |
| 841 | caaaaaaaaa aaaagagaaa aaagagaaaa aggaaagaag gctctattgg agcctggata |
| 901 | gggggaaata caccagagag ggacaggggt caaaacagga aagtcacagg aaccagaatt |
| 961 | ggtaagaggt aggaaaatct tgtgttctgt tttcctgatt aatcatcagg ggccaccaca |
| 1021 | ttttgaatat gataataata actgtatcag atgacacttc acataaaann nnnnnnnnnn |
| 1081 | nnnnnnnnnn nnnnnnnnnn nnnnnnnnnn nnnnnnnnnn nnnnnnnnnn nnnnnnnnnn |
| 1141 | nnnnnnnnnn nnnnnnnnnn nnnnnnnnaa aaatgccaag gtcagaagtg ttgaaggaat |
| 1201 | gggggtcatg gggctgacct tgacctagta ggatagtagg acacacacac atacacacac |
| 1261 | acagacacac atgccgcttt tgtgtgtgtg tgtgcgtgtg tttgtatgtg tgtgtgtctg |
| 1321 | tgtcttcaag gctgaggact gaagagaact tctcaggacc cagggcccca tgttttcaca |
| 1381 | ccaatacata gctctcaata ttgactgatg ctctctccac ctcctagcat cacttttaaa |
| 1441 | cttctggaac ccacccacga ctgcccaagt cacgattgaa gcccagccaa ccaaagtttc |
| 1501 | tgaggggaag gatgttcttc tacttgtcca caatttgccc cagaatctta ctggctacat |
| 1561 | ctggtacaaa gggcaaatca gggacctcta ccattacatt acatcatatg tagtagacgg |
| 1621 | tcaaataatt atatatgggc ctgcatacag tggacgagaa acaatatatt ccaatgcatc |
| 1681 | cctgctgatc cagaatgtca cccaggaaga cgcaggatcc tacaccttac acatcataat |
| 1741 | gggaggtgat gagaatagag gagtaactgg acatttcacc ttcaccttat atcgtgagtg |
| 1801 | attccacatg atccctgggt gttgggggac aggggtcact tctacttcac acacacagga |
| 1861 | ttgtcaggcc tggacattgc ctgtgtccct ctctgcatta tgtcccatgc tggggtttgg |
| 1921 | gcatttagtg caggacacac acagaggaga caaatttcaa cagatcagaa ttc |

An exemplary human HSPB3 amino acid sequence is set forth below (SEQ ID NO: 154; GenBank Accession No: Q12988.2, Version 2, incorporated herein by reference):

| | |
|---|---|
| 1 | makiilrhli eipvryqeef eargledcrl dhalyalpgp tivdlrktra aqsppvdsaa |
| 61 | etppregksh fqilldvvqf lpediiiqtf egwllikaqh gtrmdehgfi srsftrqykl |
| 121 | pdgveikdls avlchdgilv vevkdpvgtk |

An exemplary human HSPB3 nucleic acid sequence is set forth below (SEQ ID NO: 155; GenBank Accession No: CR450314.1, Version 1, incorporated herein by reference):

| | |
|---|---|
| 1 | atggcaaaaa tcattttgag gcacctcata gagattccag tgcgttacca ggaagagttt |
| 61 | gaagctcgag gtctagaaga ctgcaggctg gatcatgctt tatatgcact gcctgggcca |
| 121 | accatcgtgg acctgaggaa aaccagggca gcgcagtctc ctccagtgga ctcagcggca |
| 181 | gagacgccac cccgagaagg caaatcccac tttcagatcc tgctggacgt ggtccagttc |
| 241 | ctccctgaag acatcatcat tcagaccttc gaaggctggc tgctgataaa agcacaacac |
| 301 | ggaaccagaa tggatgagca cggttttatc tcaagaagct tcacccgaca gtacaaacta |
| 361 | ccagatggtg tggaaatcaa agatttgtct gcagtcctct gtcatgatgg aattttggtg |
| 421 | gtggaagtaa aggatccagt tgggactaag |

An exemplary human GJB6 amino acid sequence is set forth below (SEQ ID NO: 156; GenBank Accession No: 095452.2, Version 2, incorporated herein by reference):

| | |
|---|---|
| 1 | mdwgtlhtfi ggvnkhstsi gkvwitvifi frvmilvvaa qevwgdeqed fvcntlqpgc |
| 61 | knvcydhffp vshirlwalq lifvstpall vamhvayyrh ettrkfrrge krndfkdied |
| 121 | ikkqkvrieg slwwtytssi ffriifeaaf myvfyflyng yhlpwvlkcg idpcpnlvdc |
| 181 | fisrptektv ftifmisasv icmllnvael cylllkvcfr rskraqtqkn hpnhalkesk |
| 241 | qnemnelisd sgqnaitgfp s |

An exemplary human GJB6 nucleic acid sequence is set forth below (SEQ ID NO: 157; GenBank Accession No: AY297110.1, Version 1, incorporated herein by reference):

| | |
|---|---|
| 1 | atggattggg ggacgctgca cactttcatc gggggtgtca acaaacactc caccagcatc |
| 61 | gggaaggtgt ggatcacagt catctttatt ttccgagtca tgatcctcgt ggtggctgcc |
| 121 | caggaagtgt ggggtgacga gcaagaggac ttcgtctgca acacactgca accgggatgc |
| 181 | aaaaatgtgt gctatgacca ctttttcccg gtgtcccaca tccggctgtg ggccctccag |
| 241 | ctgatcttcg tctccacccc agcgctgctg gtggccatgc atgtggccta ctacaggcac |
| 301 | gaaaccactc gcaagttcag gcgaggagag aagaggaatg atttcaaaga catagaggac |
| 361 | attaaaaagc agaaggttcg gatagagggg tcgctgtggt ggacgtacac cagcagcatc |
| 421 | tttttccgaa tcatctttga agcagccttt atgtatgtgt tttacttcct ttacaatggg |
| 481 | taccacctgc cctgggtgtt gaaatgtggg attgacccct gccccaacct tgttgactgc |
| 541 | tttatttcta ggccaacaga gaagaccgtg tttaccattt ttatgatttc tgcgtctgtg |
| 601 | atttgcatgc tgcttaacgt ggcagagttg tgctacctgc tgctgaaagt gtgttttagg |
| 661 | agatcaaaga gagcacagac gcaaaaaaat caccccaatc atgccctaaa ggagagtaag |
| 721 | cagaatgaaa tgaatgagct gatttcagat agtggtcaaa atgcaatcac aggtttccca |
| 781 | agctaa |

An exemplary human MAGEA1 amino acid sequence is set forth below (SEQ ID NO: 158; GenBank Accession No: P43355.1, Version 1, incorporated herein by reference):

| | |
|---|---|
| 1 | msleqrslhc kpeealeaqq ealglvcvqa atssssplvl gtleevptag stdppqspqg |
| 61 | asafpttinf trqrqpsegs ssreeegpst scileslfra vitkkvadlv gflllkyrar |
| 121 | epvtkaemle sviknykhcf peifgkases lqlvfgidvk eadptghsyv lvtclglsyd |
| 181 | gllgdnqimp ktgfliivlv miamegghap eeeiweelsv mevydgrehs aygeprkllt |
| 241 | qdlvgekyle yrqvpdsdpa ryeflwgpra laetsyvkvl eyvikvsarv rfffpslrea |
| 301 | alreeeegv |

An exemplary human MAGEA1 nucleic acid sequence is set forth below (SEQ ID NO: 159; GenBank Accession No: NM_004988.4, Version 4, incorporated herein by reference):

| | |
|---|---|
| 1 | agagagaagc gaggtttcca ttctgaggga cggcgtagag ttcggccgaa ggaacctgac |
| 61 | ccaggctctg tgaggaggca aggttttcag gggacaggcc aacccagagg acaggattcc |
| 121 | ctggaggcca cagaggagca ccaaggagaa gatctgcctg tgggtcttca ttgcccagct |
| 181 | cctgcccaca ctcctgcctg ctgccctgac gagagtcatc atgtctcttg agcagaggag |
| 241 | tctgcactgc aagcctgagg aagcccttga ggcccaacaa gaggccctgg gcctggtgtg |
| 301 | tgtgcaggct gccacctcct cctcctctcc tctggtcctg ggcaccctgg aggaggtgcc |
| 361 | cactgctggg tcaacagatc ctccccagag tcctcaggga gcctccgcct ttcccactac |
| 421 | catcaacttc actcgacaga ggcaacccag tgagggttcc agcagccgtg aagaggaggg |
| 481 | gccaagcacc tcttgtatcc tggagtcctt gttccgagca gtaatcacta agaaggtggc |
| 541 | tgatttggtt ggttttctgc tcctcaaata tcgagccagg gagccagtca caaaggcaga |
| 601 | aatgctggag agtgtcatca aaaattacaa gcactgtttt cctgagatct tcggcaaagc |
| 661 | ctctgagtcc ttgcagctgg tctttggcat tgacgtgaag gaagcagacc ccaccggcca |
| 721 | ctcctatgtc cttgtcacct gcctaggtct ctcctatgat ggcctgctgg gtgataatca |
| 781 | gatcatgccc aagacaggct tcctgataat tgtcctggtc atgattgcaa tggagggcgg |
| 841 | ccatgctcct gaggaggaaa tctgggagga gctgagtgtg atggaggtgt atgatgggag |
| 901 | ggagcacagt gcctatgggg agcccaggaa gctgctcacc caagatttgg tgcaggaaaa |
| 961 | gtacctggag taccggcagg tgccggacag tgatcccgca cgctatgagt tcctgtgggg |
| 1021 | tccaagggcc ctcgctgaaa ccagctatgt gaaagtcctt gagtatgtga tcaaggtcag |
| 1081 | tgcaagagtt cgctttttct tcccatccct gcgtgaagca gctttgagag aggaggaaga |
| 1141 | gggagtctga gcatgagttg cagccaaggc cagtgggagg gggactgggc cagtgcacct |
| 1201 | tccagggccg cgtccagcag cttcccctgc ctcgtgtgac atgaggccca ttcttcactc |
| 1261 | tgaagagagc ggtcagtgtt ctcagtagta ggtttctgtt ctattgggtg acttggagat |
| 1321 | ttatctttgt tctcttttgg aattgttcaa atgttttttt ttaagggatg gttgaatgaa |
| 1381 | cttcagcatc caagtttatg aatgacagca gtcacacagt tctgtgtata tagtttaagg |
| 1441 | gtaagagtct tgtgttttat tcagattggg aaatccattc tattttgtga attgggataa |
| 1501 | taacagcagt ggaataagta cttagaaatg tgaaaaatga gcagtaaaat agatgagata |
| 1561 | aagaactaaa gaaattaaga gatagtcaat tcttgcctta tacctcagtc tattctgtaa |
| 1621 | aatttttaaa gatatatgca tacctggatt tccttggctt ctttgagaat gtaagagaaa |
| 1681 | ttaaatctga ataaagaatt cttcctgtta aaaaaaaaaa aaaaaaaaaa aaaaaaaaaa |
| 1741 | aaaaaaaaaa aaaaa |

An exemplary human MAGEA11 amino acid sequence is set forth below (SEQ ID NO: 160; GenBank Accession No: P43364.2, Version 2, incorporated herein by reference):

| | |
|---|---|
| 1 | metqfrrggl gcspasikrk kkredsgdfg lqvstmfsed dfqsterapy gpqlqwsqdl |
| 61 | prvqvfreqa nledrsprrt qritggeqvl wgpitqifpt vrpadltrvi mpleqrsqhc |
| 121 | kpeeglqaqe edlglvgaqa lqaeeqeaaf fsstlnvgtl eelpaaesps ppqspqeesf |
| 181 | sptamdaifg slsdegsgsq ekegpstspd lidpesfsqd ilhdkiidlv hlllrkyrvk |
| 241 | glitkaemlg sviknyedyf peifreasvc mqllfgidvk evdptshsyv lvtslnlsyd |
| 301 | giqcneqsmp ksglliivlg vifmegncip eevmwevlsi mgvyagrehf lfgepkrllt |
| 361 | qnwvqekylv yrqvpgtdpa cyeflwgpra haetskmkvl eyianangrd ptsypslyed |
| 421 | alreegegv |

An exemplary human MAGEA11 nucleic acid sequence is set forth below (SEQ ID NO: 161; GenBank Accession No: AY747607.1, Version 1, incorporated herein by reference):

| | |
|---|---|
| 1 | gagtggcaga gggcagcggg tccaggctcc atgaggaggc aagccttggg aatctgaggg |
| 61 | atggagactc agttccgcag agggggtctg gggtgcagcc ctgccagcat caagaggaag |
| 121 | aagaagaggg aggactcagg agactttgga ctccaggtga gcactatgtt ctcagaggac |
| 181 | gacttccagt caacagaaag agccccatat ggtccacaac tacagtggtc ccaggatctg |
| 241 | ccaagagtcc aggtttttag agaacaggcc aacctggagg acaggagtcc caggagaacc |
| 301 | cagaggatca ctggaggaga acaagtgctg tggggcccca tcacccagat atttcccaca |
| 361 | gttcggcctg ctgacctaac cagagtcatc atgcctcttg agcaaagaag tcagcactgc |
| 421 | aagcctgagg aaggccttca ggcccaagaa gaagacctgg gcctggtggg tgcacaggct |
| 481 | ctccaagctg aggagcagga ggctgccttc ttctcctcta ctctgaatgt gggcactcta |
| 541 | gaggagttgc ctgctgctga gtcaccaagt cctccccaga gtcctcagga agagtccttc |
| 601 | tctcccactg ccatggatgc catctttggg agcctatctg atgagggctc tggcagccaa |
| 661 | gaaaaggagg ggccaagtac ctcgcctgac ctgatagacc ctgagtcctt ttcccaagat |
| 721 | atactacatg acaagataat tgatttggtt catttattgc tccgcaagta tcgagtcaag |
| 781 | gggctgatca caaaggcaga aatgctgggg agtgtcatca aaaattatga ggactacttt |
| 841 | cctgagatat ttagggaagc ctctgtatgc atgcaactgc tctttggcat tgatgtgaag |
| 901 | gaagtggacc ccactagcca ctcctatgtc cttgtcacct ccctcaacct ctcttatgat |
| 961 | ggcatacagt gtaatgagca gagcatgccc aagtctggcc tcctgataat agtcctgggt |
| 1021 | gtaatcttca tggaggggaa ctgcatccct gaagaggtta tgtgggaagt cctgagcatt |
| 1081 | atgggggtgt atgctggaag ggagcacttc ctctttgggg agcccaagag gctccttacc |
| 1141 | caaaattggg tgcaggaaaa gtacctggtg taccggcagg tgcccggcac tgatcctgca |
| 1201 | tgctatgagt tcctgtgggg tccaagggcc cacgctgaga ccagcaagat gaaagttctt |
| 1261 | gagtacatag ccaatgccaa tgggagggat cccacttctt acccatccct gtatgaagat |
| 1321 | gctttgagag aggagggaga gggagtctga gcatgagatg caaccagggc cagcgggcag |
| 1381 | ggaaatgggc caatgcatgc ttcagggcca cacccagcag tttccctgtc ctgtgtgaaa |
| 1441 | tcaggcccat tcttccctct gtgtttgatg agagaagtca gtgttctcag tagtagaagg |
| 1501 | cacagtgaat ggaagggaac acattgtata ctgcctttag gtttctcttc catcgggtga |
| 1561 | cttggagatt tctttttgtt tccctttggt aattttcaaa tattgttcct gtaataaaag |
| 1621 | ttttagttag cttcaacatc taagtgtatg gatgatactg accacacatg ttgttttgct |
| 1681 | tatccatttc aagtgcaagt gtttgccatt ttgtaaaaca ttttgggaaa tcttccatct |
| 1741 | tgctgtgatt tgcaataggt attttcttgg agaatgtaag aacttaacaa taaagctgaa |
| 1801 | ctggtgttgt gaaacagaga aaaaaaaaaa aaaaaaa |

An exemplary human MAGEA9B amino acid sequence is set forth below (SEQ ID NO: 162; GenBank Accession No: NP_001074259.1, Version 1, incorporated herein by reference):

| | |
|---|---|
| 1 | msleqrsphc kpdedleaqg edlglmgaqe ptgeeeetts ssdskeeevs aagsssppqs |
| 61 | pqggasssis vyytlwsqfd egsssqeeee psssvdpaql efmfqealkl kvaelvhfll |
| 121 | hkyrvkepvt kaemlesvik nykryfpvif gkasefmqvi fgtdvkevdp aghsyilvta |
| 181 | lglscdsmlg dghsmpkaal liivlgvilt kdncapeevi wealsvmgvy vgkehmfyge |
| 241 | prklltqdwv genyleyrqv pgsdpahyef lwgskahaet syekvinylv mlnarepicy |
| 301 | pslyeevlge eqegv |

An exemplary human MAGEA9B nucleic acid sequence is set forth below (SEQ ID NO: 163; GenBank Accession No: NM_001080790.1, Version 1, incorporated herein by reference):

| | |
|---|---|
| 1 | gtgcgcactg ggggtcagag agaagggaga ggcctccttc tgaggggcgg cttgataccg |
| 61 | gtggaggagc tccaggaagc aggcaggcct tggtctgaga cagtgtcctc aggtcgcaga |
| 121 | gcagaggaga cccaggcagt gtcagcagtg aaggttctcg ggacaggcta accaggagga |
| 181 | caggagcccc aagaggcccc agagcagcac tgacgaagac ctgcctgtgg gtctccatcg |
| 241 | cccagctcct gcccacgctc ctgactgctg ccctgaccag agtcatcatg tctctcgagc |
| 301 | agaggagtcc gcactgcaag cctgatgaag accttgaagc ccaaggagag gacttgggcc |
| 361 | tgatgggtgc acaggaaccc acaggcgagg aggaggagac tacctcctcc tctgacagca |
| 421 | aggaggagga ggtgtctgct gctgggtcat caagtcctcc ccagagtcct cagggaggcg |
| 481 | cttcctcctc catttccgtc tactacactt tatggagcca attcgatgag ggctccagca |
| 541 | gtcaagaaga ggaagagcca agctcctcgg tcgacccagc tcagctggag ttcatgttcc |
| 601 | aagaagcact gaaattgaag gtggctgagt tggttcattt cctgctccac aaatatcgag |
| 661 | tcaaggagcc ggtcacaaag gcagaaatgc tggagagcgt catcaaaaat tacaagcgct |
| 721 | actttcctgt gatcttcggc aaagcctccg agttcatgca ggtgatcttt ggcactgatg |
| 781 | tgaaggaggt ggaccccgcc ggccactcct acatccttgt cactgctctt ggcctctcgt |
| 841 | gcgatagcat gctgggtgat ggtcatagca tgcccaaggc cgccctcctg atcattgtcc |
| 901 | tgggtgtgat cctaaccaaa gacaactgcg cccctgaaga ggttatctgg gaagcgttga |
| 961 | gtgtgatggg ggtgtatgtt gggaaggagc acatgttcta cggggagccc aggaagctgc |
| 1021 | tcacccaaga ttgggtgcag gaaaactacc tggagtaccg gcaggtgccc ggcagtgatc |
| 1081 | ctgcgcacta cgagttcctg tggggttcca aggcccacgc tgaaaccagc tatgagaagg |
| 1141 | tcataaatta tttggtcatg ctcaatgcaa gagagcccat ctgctaccca tccctttatg |
| 1201 | aagaggtttt gggagaggag caagagggag tctgagcacc agccgcagcc ggggccaaag |
| 1261 | tttgtggggt cagggcccca tccagcagct gccctgcccc atgtgacatg aggcccattc |
| 1321 | ttggctctgt gtttgaagag agcaatcagt gttctcagtg gcagtgggtg gaagtgagca |
| 1381 | cactgtatgt catctctggg ttccttgtct attgggtgat ttggagattt atccttgctc |
| 1441 | ccttttggaa ttgttcaaat gttcttttaa tggtcagttt aatgaacttc accatcgaag |
| 1501 | ttaatgaatg acagtagtca cacatattgc tgtttatgtt atttaggagt aagattcttg |
| 1561 | cttttgagtc acatggggaa atccctgtta ttttgtgaat tgggacaaga taacatagca |
| 1621 | gaggaattaa taattttttt gaaacttgaa cttagcagca aaatagagct cataaagaaa |
| 1681 | tagtgaaatg aaaatgtagt taattcttgc cttatacctc tttctctctc ctgtaaaatt |
| 1741 | aaaatatata catgtatacc tggatttgct tggcttcttt gagcatgtaa gagaaataaa |
| 1801 | aattgaaaga ataa |

An exemplary human TRPM1 amino acid sequence is set forth below (SEQ ID NO: 164; GenBank Accession No: AAH58286.1, Version 1, incorporated herein by reference):

| | |
|---|---|
| 1 | mkdsnrcccg qftnqhippl psatpsknee enkqvetqpe kwsvakhtqs yptdsygvle |
| 61 | fqgggysnka mvrkafrhga tritafiggq spspklqipg llhgcgsifl dislknqeiy |
| 121 | lctwllamrl gnwtpl |

An exemplary human TRPM1 nucleic acid sequence is set forth below (SEQ ID NO: 165; GenBank Accession No: BC058286.1, Version 1, incorporated herein by reference):

| | |
|---|---|
| 1 | ggggtccctg ccctggccaa ggaggaggct gaaagagcct gagctgtgcc ctctccattc |
| 61 | cactgctgtg gcagggtcag aaatcttgga tagagaaaac cttttgcaaa cgggaatgta |
| 121 | tctttgtaat tcctagcatg aaagactcta acaggtgttg ctgtggccag ttcaccaacc |
| 181 | agcatatccc ccctctgcca agtgcaacac ccagcaaaaa tgaagaggaa aacaaacagg |
| 241 | tggagactca gcctgagaaa tggtctgttg ccaagcacac ccagagctac ccaacagatt |
| 301 | cctatggagt tcttgaattc cagggtggcg gatattccaa taaagccatg gtgagaaagg |
| 361 | cattcagaca tggtgccact aggatcacag ctttcattgg cggccagtct cccagcccca |
| 421 | aactgcagat acctggtctt cttcatggct gtggctcaat cttcctagat atttcattga |
| 481 | aaaaccaaga gatatatctg tgcacatggc ttttagccat gaggcttgga aactggacac |
| 541 | cactgtaaag aacatctagt gtcccgtaaa tccataccaa agctctgaat ccacaaacca |
| 601 | ggctctggcc caaccctgca aacacactcc attgctccat cttcagtaaa ggaagacaaa |
| 661 | ttcatttttc taataactgt ggacctgcag cccccttaga tgtgttgaga gtctttggaa |
| 721 | atattttcct ctgaggtctg tccacagctt ccctgggcct gcgctcagct ggcccgagaa |
| 781 | ggaccaaggt ccctcacatt tgcatgtaaa cagggagtgc cctctgccct tccagtgagc |
| 841 | cctgccagcg tgggggaggc ttcagctctg tgatccgttc cagctcactc tgaattacac |
| 901 | tcctacatgc ccagtcacag actttttgca atttcatttt atttcactgg cccaacatca |
| 961 | ttgttaaaat aaaatttagt tgtgttccaa atgctcaaaa aaaaaaaaaa aaaaaaaaaa |
| 1021 | aaaaa |

An exemplary human CSAG4 nucleic acid sequence is set forth below (SEQ ID NO: 166; GenBank Accession No: NR_073432.1, Version 1, incorporated herein by reference):

| | |
|---|---|
| 1 | gggaagcagg cgcaggctcc gtgaggaggc aagaggaaag gccaagaact gacaaattaa |
| 61 | taaagggcca tctacaagcc aggaataagg ccatcaccag aagacaaccc cgccagtcct |
| 121 | tgatctggga cttccacatt tcaaaactga aaaaagataa atttctgttg tttcagctac |
| 181 | tcagtcaatg gcattttgtt atggcagcct aagctaagac ataacctcag cctgctggcc |
| 241 | tgccttcact gtcctggggg aggcatggag agaccaggtg gactggagta gactgttgag |
| 301 | agacactggt ctggtgaaga tgtccaggaa accacgagcc tccagcccat tgtccaacaa |
| 361 | ccacccacca acaccaaaga ggttgccaag acaacccgga agggaacagg gacccgtcaa |
| 421 | ggaagttcca ggaacaaaag gctctcccta aaagaccgcc gcttcaaaaa aacctgagga |
| 481 | atggagtggg ccaacactat ccagccactc cgaccagcca aaagaactca atcaaaatga |
| 541 | gacacagtag gaccacaagg gcaaggagac caccaccttc tccagtctct ctttgggcag |
| 601 | ccagtaattc ccgggcaagg ccagaaacct caaggctacc tgaaaagtct ccagaggtct |
| 661 | aaccccagaa aaatagccaa cagggtgtag agtacatttt ataccccaaa gggtataccc |
| 721 | catggtgacg aaaataaaat gaacatgttg taaaaaaaaa aaaaaaaaaa aaaaaa |

An exemplary human AC093787 (RP11-215P9) nucleic acid sequence is set forth below (SEQ ID NO: 167; GenBank Accession No: AC093787.1, Version 1, incorporated herein by reference):

| | |
|---|---|
| 1 | tagttgcttc actttccctt gtctagaaaa gattgctgta gtaattatta ttaacactct |
| 61 | taataataat ggtaaccagt aataataact gagccttttt acatcccagg gttagtatta |
| 121 | taattggtgc cttatgtatt aattcttctg atcctcacca gaggaagctc acaaaggtaa |
| 181 | ctaactggct aagcctctca taggcagtag gtgatacagc ctgaagaacc cagatggttc |
| 241 | agctcctgaa tccttcttct taccattatg ttctgccacg tctcacatac cagtgggaat |
| 301 | ccacacagca gatgaccctt aactttgttc tgtaaaaatt ccatagtagg taaaataagc |
| 361 | tatcacccaa ggggtttctc tactctccaa tacagaaaat ggacattagc ctagtgatac |
| 421 | caaaatgcta ctgctcttcg tgtgaaatcc tagccagttg aaaccattaa aggggacaaa |
| 481 | tgtcactgag catctactgg atgccagaca ttatttcatg catttcaatg tgatttatct |
| 541 | cacttagtca aaaaaaaata tatatggtaa atttgaagaa tggttaaaga agtgggtgaa |
| 601 | cagccttggc attttctcaa cactataatt tttttggttg tttactttgt tttttcttgt |
| 661 | aattaagtaa taataaaaag ttcaataaca acagctcact tttttgaaga atggttaaag |
| 721 | aagtgggtga gcagccttgg cattttctca acactataat ttttttggtt gtttactttg |
| 781 | ttttttttct tgtaattaag taataataaa aagttcaata acaacagctc actttttttt |
| 841 | tttttttttg agacagagtc tcgctctgtc ccccaggctg gagtgcactg gcgagatctc |
| 901 | agctcactgc aagctccacc ttccaggttc acaccattct cctgcctcag cctcctgagt |
| 961 | agctgggact acaggcatcc gccaccatgc ccggctaatt ttttgtattt tttttagtcg |
| 1021 | agacggggtt tcatcatgtc agccaggatg gtctcgatct cctgacctcg tgatctgccc |
| 1081 | gcctcggcct cccaaagtgc tgggattata ggtgtgagcc accgtgccca gccaacaaca |
| 1141 | gctcactttc aaaatgtatg ttactcttta gaatgtattc aatcactaat ttttgctatg |
| 1201 | tgcgagttgt tcttatagtg gccagagtcc ctcttcaaat aggtgataaa atatcttaaa |
| 1261 | gtatgtgaat attcatatgt ctcaagttca gttaacaaac aaatataatt agattaagcc |
| 1321 | ttgatatttc taatagactt ctctaataaa tgtattttct taatttctac tattattaat |
| 1381 | gctttttatt taaaattgag gatgtgatac ataagaaata aatataacta gagccttcat |
| 1441 | atttctaagt tttctctaat aaatatattt ccttaatttc taccactatt aatgcttatt |
| 1501 | taaaattaag catgtgataa ctatttgttg ccacctcata caaaataaaa taatgtaata |
| 1561 | ttaattatgc ttattgtgca tgtgctttgt gtcgacttac ggtgtaagtt gtctagtagc |
| 1621 | tcagttagcc cccacttgcc aaactatgta gtcataccaa tttttttttc agatggataa |
| 1681 | agggaggcag gagggttttg taaccacaga gttcatcatc agaggtgaac aggaccaaga |
| 1741 | tttgaacctg acagtttttc ttcagagttg gaaatcttaa tctccataca tacttgaggt |
| 1801 | tttcggatcc tcaccaaatt ttcagttatc taacttaaat acggacaatg tgaaggtcac |
| 1861 | aaaattactt tcagtcatct ctaagataaa tctcagagag ataagcaatt tttcagcaga |
| 1921 | tgaaatataa ttaaaacaag agattacgta ccttctggtc ctgttgccaa gagatcactg |
| 1981 | gtttccccca aggcttgatc tattcatgag ggcagacgtt ggaatacttc gaatttctcc |
| 2041 | tcatctaggt aagcttcggg atgcattatg gcaaccagaa taattttatt caagaatagg |
| 2101 | aaaggccagc tagtttagca cacttagaca tgaaatttac tattatcata tttaattcaa |
| 2161 | tcttatttct tttctatttc aatttacaga tagaaattat agagtagttt tataggctga |
| 2221 | cctgtgtaaa ctccttctca taatttttta atacagtcct gaggattcta tacaataatg |
| 2281 | taattgtgtt gcctgccatt caatgagtaa ttggccttca ggtttctctt aatgatggta |
| 2341 | aataagatgt tacgatttcc tttgttttct gtaagatatt taataatgaa tgtctcaatc |
| 2401 | taccacaatg tttaactttg gggaacaaga tctgaaaaaa atatactctt aaaaacttaa |
| 2461 | atgttgaatt attccttcac atgtaatgtc ctgacactga tgtttctacc agcatacagg |
| 2521 | gagtaaatat tcaaaatgat tgcataaatg tatacaaact agaggctagg aactggtaag |
| 2581 | ccttgtccag atgacaagac actctttgct aaaattaaca ttctctctca cctcagaggt |
| 2641 | ttcatcattg ctattgttgt caatattgct gtttattaaa actgtattct gtaaattacc |
| 2701 | agtgagagtg agtggtttta atattacttt agtgtccaca tagaaattct tcagtgatga |
| 2761 | gaagacccaa aagattgctc actgtacagg gactcttggc tagctaaaaa aaaaaaaaaa |
| 2821 | aaaaaaaaaa aaaaaagcta aatatttcca ttgctgagat gtaaacgaag aattcccaaa |
| 2881 | catttgaggt atctgtttaa tgtctaaaat aagaagttga aacctttcac taacaacttg |
| 2941 | tgctgtgaaa cacataagtt taaatgagta attcaacagt tactgtttgg atttacatag |
| 3001 | aaagactttt gccaaagcct gtgctgaaca catatcaaaa caattatgcc tagcatgtac |
| 3061 | acagtgataa gaatgttttc ttttggcatc cactagagga gaaacttaga gcagcaccac |
| 3121 | tttctgccgc tacccttggg tccatgctgc ggtagttact tacctgaatt gtcctcggag |
| 3181 | taactgcatt tccttgtata tgaaatggac ctaaagtaat ggatttgccc attagtgaat |
| 3241 | aatattagaa acaatttcaa ttgtaatgca attgctgttc agttccatgc aatttctgca |
| 3301 | tagaaacaca actgaaagaa aaatgacaac caatcaacca gtaaaacggc aataaatact |
| 3361 | ttttaggtga gaaaacgtca aaagagtgtg gatttggaag tccttccatt gtgctaatta |
| 3421 | tcgatctttt ttctctctct gaacacttgt caacagtcat taggtttata caattttctc |
| 3481 | tttttaatgc atcaaaaaat caccaaatag gaagaagtaa atctgtataa cgggacatct |
| 3541 | aacaattggt tttaaaatac ataacctaca gtctaaaata aagaagaaat atatacatgt |
| 3601 | gaagcttttt tttgactgac tatattataa aatatgctaa aattgtactg caaatgaact |
| 3661 | ctcctcacct cccaaataat aacaaaacat acagtgaaac aaaattgtac aagtttatat |
| 3721 | taaggctctc aaatttaaac taagcataaa aactgagcaa tttatcttgg gattctaaaa |
| 3781 | aataaaaatg aatctaacaa aattcaaatt gatgataccc gacatgttgt cataatataa |
| 3841 | caaagtttac tctctgaaga aatatcaagt tttaaaaata aataggaaca aaaccaagca |
| 3901 | tcatctaaag gccagtttct agttttctca gcaccatggg taacttttgt aagaaatgag |
| 3961 | ataatcctgt agtcccagct acttgggggg ctgaggcagg agaatggcca gaacccggga |
| 4021 | ggcggagctt gcagtgagcg gagatcgcac cactgcattc cggcctgggt gaaagagcga |
| 4081 | gactccgtct caaaaaaaaa agaaatgaga taattagatt taccgcaaat gaaattttga |
| 4141 | tgaagtgcta ttgaagtata atattctatg aaatcccttg ttaccttgaa aggagctaaa |
| 4201 | tgacaactgc ttgattatca tgggacaaag ataactttag gtttttttct tctaaattgt |
| 4261 | ggatagtgac tatttcggta ttttctgtaa tggtattttc tgtaagtgat attacccagc |
| 4321 | ttttaggtcc acatcttagc caaatcactt cctgaaacct gacttacaac aaaaaccttc |
| 4381 | tttgttcttc cttagcctgt gaatagtggc agcatttaaa attaatacca tgcaattacc |
| 4441 | ttctgttcta tgataatctt tcatattttc tcttgttttc tcaaactctt cataactctt |
| 4501 | aggtcaaatt tcatgcacta tatagttccc ttccttttgt atcaccttta tgtagctaag |
| 4561 | attatagtaa gttgttaaca agaatttata gatttatcgg tcaaattatt aaccgttcaa |
| 4621 | caagaagacc aaagccaatg taatattttt aaaagtaggg attaagaaaa ttacctttgg |
| 4681 | tattatcctt aaagagagtt ttagtcctta ttttccaata catacactca actatacctc |
| 4741 | ccgttttgta agaaaaccac ctgcattagg gaacatttct taaattgttg aagtcttaac |
| 4801 | attgatacat ttaactattt acttacaatc tttgggaata ttatagagga tattcactaa |
| 4861 | agataattaa agagattgta ctccacatgt cagatgaatg gctttaaaac cttagtagaa |
| 4921 | ttcaccagga tttttgctta aagtgcaaat ttctcagtta gttccaagtc cagagattct |
| 4981 | gatttaacag gcatagaaca ggacctggga atctgcattt taaataaact caaggtccat |
| 5041 | cttaaatgca ctcagaaata agtgatttta ctacttgaga aatgtaactt gaggagttta |
| 5101 | aaatctgtga aatctgtgat aaaaattaat tatgtggctg ggcatggtgg ctcatgcctg |
| 5161 | taatcccaac actttgggag gccaaggcag gcagatcaca aggtcaggag ttcaagacca |
| 5221 | gcatggccaa tatggtaaaa ccccgtcttt actaaaaata caaaaaaatg taaccaggtg |
| 5281 | tggtggctgg tgccagctac ttgggaggct gaggcaggag aattgcttga accggggagg |
| 5341 | tggaggttgc agtgagccta gattgcacca ctgcactgca gcctgggtga cagagtgaga |
| 5401 | ctccagttca aaaaaaaaaa aaaaattaaa tatgtgcttt ggcactttaa agagaagtaa |
| 5461 | ataaatgatt cgtgtttggc ttctattctg tccatctctg ctaattttct ttctttcttt |
| 5521 | attcaccttg gctggtctgt gtatttacat gtaactttca tgaaaactcc atccatcttc |
| 5581 | atcaatacac ataatacata ccttgcaagg ccctttttag aagtcctatt caaatatttc |
| 5641 | tgagccagat gaatcagtct gtgttagtct gttttcatgc aaataaagac atacttgacg |
| 5701 | ggtcatttat aaagaacaag tgatttagtg gactcacagt ttcacatggc tggggaggcc |
| 5761 | tcacagtttc acatggctgg gaaggcctca caatcatggc agaaagcaaa ggaggagcaa |
| 5821 | aggcatgtct tagatggcag caggcaaaag agcttgtgca ggtgaactcc cattatgaaa |
| 5881 | ccaacaggga cttattcatt accatgagaa cagtatgggg gagaccgccc ccatgattca |
| 5941 | attatctcca cctggccctg cccttgacat attgagatta ttacaattca aggtgagatt |
| 6001 | tgggtgggga cagagccaga ccatatcaca gtcccataat ctcttttgta gtgataaaaa |
| 6061 | tcacaagtct gaaccattca tgatattttc ttatactata atttatgaaa gacttacgga |
| 6121 | agattttttt gtatctcctc caccttcact attgcaatgg attaaatggt tgtttttctc |
| 6181 | cccccaccca aattcatatg tcaaaatcct aacccacaat gtatggtact aggaggtggg |
| 6241 | gtctttggga gcaaattaca tcatgagact ggagcccatg tgaatgagat tagcaccctt |
| 6301 | ataaaaaaga ccccagagaa ctctcttgct ctttttctat tgtgtgagga tataaagaaa |
| 6361 | agacggcagt ctacaagtca gaagagggtc ctcacaagaa ttaatccatg ctggcactct |
| 6421 | aatctcatat ttctgaactc aagatctgtg agaaataaat ttctgttgtt tataaaccca |
| 6481 | tccagtttac aaagtgccta gtctatggta ctttgctctg gcagcacaaa ctaaaacaac |
| 6541 | tatattttta agaaaaaaaa aatgcatgaa catacatcta taaaagtctt caacctgata |
| 6601 | actctgaaat aacttttact ttaatggtat tcatataaga aagaaatgta tgaagccaaa |
| 6661 | ataaatctca aaattaaggg ggaatattac ttaccaaaat gcatgtttgc tttgccaaag |
| 6721 | cctgaaactt ttttcttttt aagcaaattg aaaacctcaa agatttcaat attttttatt |
| 6781 | acttatccca gcaaaatcat tcagaccact tatttttaaa attatatagt cactgattta |
| 6841 | tttcactttt atgcagttag gataggaagt ggaggtcaat tggaaaatat aattatagga |
| 6901 | ataggaccat catgaaatga ttttttaatt tattaccatt cagagcatct atacttgttt |
| 6961 | gtcacacatt cagaagaatg gaagaaagga gggtagagaa tgagtcatat gcattgaaaa |
| 7021 | ttattattca gtttcaaaat gagtgcccaa aactatagac actgtaagac actgaatttg |
| 7081 | aatattttac atttcattct tctattaaaa ttcatttcat tgagaacaaa aagaaaacaa |
| 7141 | gaaaaaataa gaagataagc atcttcagag aaacaactac agcccgaaac catgaactga |
| 7201 | gaagcaaacc ttccaaatac tgttatattt caactaaaac aaggagtgat cagagtgaat |
| 7261 | gctttcacat gtataatcag tacatgttca ggatggtcat tttttccagc tatcattgcc |
| 7321 | actactctgg gagatgtaac tcaaaaacta tttttcttaa aattgttaga ttttatggaa |
| 7381 | taatgtgacc atgatattaa taaaagtagg aaagtcacta tagagtaact cagtgttgtc |
| 7441 | taaccataaa atggaaagaa tttgaagttg taagagaatt caggctgatg tactctattc |
| 7501 | atttcatata ctctgagttc caaacagaat gaagctcaca gaagtggaga ggagcaaaag |
| 7561 | agaaaagcat taaccagcat gtgataatat agagattaga aatgtagatt tagaaaccta |
| 7621 | ggaatctaaa tatgagccac attaccttta gaaaagtgaa tgctgatagg aggagaaaca |
| 7681 | agtgtccagt acttagggag taaggaaatt agtgtgacag tattccatta caaaaacatg |
| 7741 | gatttaaaaa aaggtacaaa ctgcaatgcc acaccgctcc tgccccagag gatcccttag |
| 7801 | ctaatacctg gatctgttct atgaggaatg cagagagaaa aaaatcagta aaatatatat |
| 7861 | gcctggtgtt ctattttcta aatatgaaaa tgaacaaaaa gcataaacaa gatgcagttg |
| 7921 | ctaaaactaa atcacaagta gaattaccaa agtaatcaga gaacaaattg aagttaactt |
| 7981 | aatctcaatt tagaaattga agtgaacaac taaggaatta tataaaaaag aaatacatag |
| 8041 | gcactataca tcagcaatta catccagaaa aatagtgaat atctagaaga aaccacacaa |
| 8101 | aagtaaatta tatgaaaaga caaaggcata ataaaaatcc ttaatatggc attggtaaat |
| 8161 | atagatgcat gacaatggag actcaacctc tgcctacttg atgttttgaa gaataagaaa |
| 8221 | gaacattaat actggaaaca ttacttgttg aaaacttcaa agagataaaa tgacacttga |
| 8281 | atctatagat taaaatagca ttctgttttt tagacaaaaa tatagagata ctgatcatca |
| 8341 | ctaagactga ctttgattca cttactagat ttgaagaata aagcaatagg ataattgtca |
| 8401 | tctgggcagt taaacaagtc tctgagaatt caactgttgc cgataaagaa ttaatgatta |
| 8461 | ccagaattat gcaacttttg taaacgtcta gaaatccaga acaagtatat aaactgtttt |
| 8521 | tattgcatga aaaaaatagg cagcccaaaa ctgtcatttc tgagagaaag aaaacaatat |
| 8581 | atacccttta ttgtgccagc atacttactg aaagtgattt ccagcctcca gcatggtatg |
| 8641 | agacaatccg agagagaata tcatgatttg ctgaatttaa gagcaagata tcaaagttta |
| 8701 | tgtaggccaa ggtgactaga atcatcaagg cagactgaca gagagaagag ggctgcacag |
| 8761 | agagagaact gtggagattt acatagaggt tctctggagt ccttgagtga gaactgaact |
| 8821 | gtgcctacat aaagtaaaac tccatgagat agaaaaatga gtcttcatta gcaatgggcc |
| 8881 | agagaaattc ctggaggtca tatgggcctt agaagaaccc gtatgtgctc cagccagagt |
| 8941 | gagaaactta taatagcaga tagagttctc agaataatca caccttagta gcggggtgaa |
| 9001 | tcagttctag agtcaaggct ctgaagccat tataatcaca tttaaaagca aacaatatca |
| 9061 | ccattgatct cagataactt aactgcattt gaaaacaaag ttcatctctc tttaaagaaa |
| 9121 | taaaacaaaa tccagatcct aacaatgtag aattcagaat ttcccttaga cattaaaaaa |
| 9181 | ataacagaaa tgcatataaa caaaaaaatt gatccaaaac aagacaaaaa ttaatcaaaa |
| 9241 | cagaccaaga aaagatagaa attatgggat tagaaaatat taagcagctt aacaacacac |
| 9301 | ataattgaaa tcccaggaaa tgggggcagg gcaaaaacta cttgtaaagg tagtggttga |
| 9361 | gatattttta aaacttggta aaactataaa ccaacaggta aaagaagtgc agtggactca |
| 9421 | aagcaaaatg aataaaagga ttaagttgaa gaaagttgtt tttaaaaaga aaacttaaaa |
| 9481 | acagttggag aaaaagagat gtattactga caatggaacc aatgtaataa ctacttttta |
| 9541 | tgataagcat gtatatcaga agacagtagc gcaacattct attacttaaa gcaagagaaa |
| 9601 | caagtcaacc tggaaattca ttcaagtgaa aattctaaaa aaatcaagaa atttacaaaa |
| 9661 | atgaaagcaa ataaagctat tttgacaata agtagctgat agattttatc aacaacatac |
| 9721 | tgggactgaa agaaatggta gacaaaaaga aagtaacagg atgataatgt acttttttta |
| 9781 | ttattatact ttaagtttta gggtacatgt gcacaacgtg caagtttgtt acatatgtat |
| 9841 | atatgtgcca tgttggtgtg ctgcacccat taactcgtca tttaacatta ggtatatctc |
| 9901 | ctaatgctat ccctcccccc tccccccacc ccacaacagg ccttggtgtg tgatgttccc |
| 9961 | cttcctgtgt tctcattgtt caattcccac ctatgagtga gaacatgcgg tgtttgtttt |
| 10021 | tttgtccctg cgatagtttg ctgagaatga tggtttccag cttcatccat gtccctataa |
| 10081 | aggacatgaa ctcatcgttt tttatggctg catagtattc catggtgtct atgtgccaca |
| 10141 | ttttcttaat ccagtctatc attgttggat atggggtaaa ggattcctat ttaataaatg |
| 10201 | gtgctgggat aatttacatt tttaaaaaga aatgattcat gttagaaata atacataggg |
| 10261 | cagtgcatgg tggctcacgc ctgtaatttc agcactttgg gaggttgagg tgggcggatc |
| 10321 | acaaggtcag gagttggaga ccagcctgac caacatggtc aaaccctgtc tctactaaaa |
| 10381 | atacaaaaat tagctgggtg tggtggcacg tgcctctaat cccacctact caggaggctg |
| 10441 | aggcaggaga gtctctcgaa cccacgaagt ggaggttaca gtgagccatg attgcaccac |
| 10501 | tgcactccag cccgggcaac agagcaagac tccatctcaa aaaaaaaaaa aaaaaaaaaa |
| 10561 | aggaaaaaga aacaatacat acaaaagaca ttttttcatg ttttaagtaa tgttaaaggt |
| 10621 | aatggattat ttaaaaccaa aataataatt atttctgtgg tattcaaaat atttataaag |
| 10681 | aaaggaggaa aaccctatga cagtagggca aagaactgta gagagaaaat ggatagacag |
| 10741 | ttataatatt tgttttttta tttgaacatt tattttaaat ttatggaaca catgtacagg |
| 10801 | tttgttacat ggatatattg cgtgatgctg aggttggtgg tacggttaag cccgtcgccc |
| 10861 | aggtactgag catagtacct aacagatagt ttttcaaccc cggtctccct ccctcccttc |
| 10921 | ctctccctct agcagtgtcc agtttctatt gttgccgttt tcatatccat gagtaccctg |
| 10981 | tgtttaactc ccatttacac gtgagaacac gtggtatttg attttctgtt tctgcattaa |
| 11041 | ttcacttaag ataatggctg caagttgcat ccatgttgct gcaaatagat ggttttgttc |
| 11101 | tttattatgg ctgtgtagta ttccatagtg tatatgtacc atattctctt tatccaatcc |
| 11161 | actgctgatg ggcacttagg ttgattccat atctttgcta ttgggaatag tgctacaatg |
| 11221 | aacatacaag tgcatgtgtc tttttggtat attttcttat ggatatataa ccagaaatgg |
| 11281 | gattattaag tcaaatggta gttctgtttt aagctctttg agaaatctcc aaactacttt |
| 11341 | ccccatgggc tgaactaatt tacattccca ccaacagtgt ataaacattc cccttcttct |
| 11401 | atagccttgg caggaacttt tcaataaaat atattattaa acatttttgg agcttttatt |
| 11461 | aatagccatt ctggctagtg tgagatgata tctcattttg gttttgattt acatttctct |
| 11521 | gagattagtg attacgagca tttttttcat gtttgttagc cacttgtgtg ccttcttttg |
| 11581 | agaagtatct gttcatggtt ttgctcattt ttaaaattat ttgttttttg cttgcttaat |
| 11641 | tatttaagtt ccttatagat tccagataag agacttttat tgaacacgtt gtttgtgaat |
| 11701 | attttcgccc atgctgtggg gtgtttgctt atgctgttga tagtttcttt tactgtgcag |
| 11761 | aagctcttta gttgaattag gtcccacttg tcaatttttg ttttgttgaa attgctttcg |
| 11821 | aggacttagt cataaattct ttcccaaggc tgattttcag aatggtgttt cctagcatta |
| 11881 | cttctaggat tcttatagtt tgaggtctta cattgcagga ttataggttg ctgggtttta |
| 11941 | gtatcatggt aatgctggct tcagaattgc aatattttaa aactatatag caggtattat |
| 12001 | aaatattatt tgaaggttga gtgttttaag ttaaaaaaaa tgtacagatg gtactcttta |
| 12061 | tgcacatcag tgcggggcct tagtaaccac tgtgcaagtt gaaactgggc aaagttatct |
| 12121 | taataattac tggaaaaaca taaacttgtt cctgcaatca ttaaaaacac ttgtcagtca |
| 12181 | ggtgcagtgg ctcgcactgg taattccagc actttgggag gccaaggtgg gcagatcact |
| 12241 | tgagcccagg agtttgagac cagtttgggc aacatgacag aaccctgtct ctacaaaaac |
| 12301 | tacaacaaat tagccaggag tggtgatacg tacctatagt cccagctatc caggaagctg |
| 12361 | aggtgcgagg atcacctcag cctggtggtc gaggctacag tgagcctttt tgataccact |
| 12421 | gcactccagc ctggacaaca gagtgaggtc ttgtctctaa ataaaagaat aaataaataa |
| 12481 | acacatttgt caaaatatta aaaatgtctt actgttggtt atatatgcat agaaaagtaa |
| 12541 | aaaccaaaaa acaaccctaa aactagtatt tacatagtac actataattt aaaacattga |
| 12601 | taattaaaat acatgtttta tatatttctt tgaaatgtat tcagattagt ttgtacagta |
| 12661 | tttgtacaac ttctcatcat gtgactctca atatttttta tgctttggtg aattgccata |
| 12721 | cttctttata aatttgatta tttgtccaac attttattgt ttgtgcgttc attgtcttta |
| 12781 | aacatcttgg agagtttctt caatgtaatt ttttttctca gcatcacatc ttctgggaaa |
| 12841 | actttcgtct tttccctgca atcagcttct tcatttaagt tgtaaatatg tattccataa |
| 12901 | gttcccctga ctgcacatat gaaatctctt ggatggtagt tgtgtcagca ttctcgcaat |
| 12961 | taactatttc ttctataaat ccatcaagat ccgatctgaa ttcactttcg gtgttgacac |
| 13021 | tttcatttat ttgccacact ttcatttttg ttggctgatt ttctgtcgtt tatcaatttt |
| 13081 | tgtgtaatgt cacgtcttta tcactgaaag acaaagaagc aacactactg tatgctttcc |
| 13141 | tatctctgta tgaatacaaa acagatacat agtaattgat tattgataga tattgagaaa |
| 13201 | agtgacatga ctaatcatgg atcttgatgc aaatctgaca tttacctaat agttttatgg |
| 13261 | actgaagagt taacagaaaa aaattgtact tcaagccatt agtcccaatt aatacactgc |
| 13321 | tgtacctaca ctgttttgtt ttggggcttg tgctatgtaa ctaaagcatg ataactaaag |
| 13381 | cctttgcata tcaaaactaa ggaaaacaag aactgtctat tttatgagcc ctatatcaat |
| 13441 | cattaaaatt aaaacaatgg aatggttaaa agctaatagt ggagataaaa gcggatctta |
| 13501 | aaaactaaat tttcaattca aaagaaggca gaaaaagggg gaaaatgaac aaaaatgatg |
| 13561 | agacatttag aaaacatata gcaagagagt agttatacac ccaatcatat tgataattgc |
| 13621 | attaaatata aattgacaaa atattcttat taaaaggcag agatcataag acaggattaa |
| 13681 | aaaccctgac tcaactatat gctctttata agaaacttac ctgaaccaaa aaaaagaaaa |
| 13741 | acaaacaaac caaaacccac taaaactgtg aagagcaaag taggcatcag aacaggaaat |
| 13801 | atcactgggg atgaagaata acatttcata atgacaaagg ggaaaataca ccaagaagac |
| 13861 | atgtaaataa taaatatgta tgcacacaat agcattactt caaaatacat aatataaaac |
| 13921 | ctattaaaac tgaaaggtaa aatagtaaaa ccacagtcat ccatggggat ttcaacagtc |
| 13981 | tcctgccaga aatttttaaa atttgttaaa caaaaaattg gtaagggtag agaggatctt |
| 14041 | aaaaatacaa ttagccaact tgatctaact gaatctttta gaataatcta aggatgaggg |
| 14101 | atgaggtagc agagaaagaa aaggcagaca tcaatgtgac attactgttt caaggctatg |
| 14161 | agaatacacc aataattgtg tgtgtgtgtg tgtgtgtgtg tgtgtgtgtg tgcagatggt |
| 14221 | aagctcaatc ttaaaaatgt tgagttttaa ctgaaaattc attattagga aagataagag |
| 14281 | gagatgatat ctagtgagag gctgtatgac tgaactctaa gagaaaggtc acagcagaaa |
| 14341 | ttgtgtacct gacagctcta gaaggaggtc agtcaaaagt aagtcagtga tgaattctct |
| 14401 | ggtgtaaaag cagaggaatg aggattagat ttaaaacaca tggaagcaga gtgacttatg |
| 14461 | ataaaaacat gagcttgaaa atcctgcaga gagggcttta aatcctgggt atgatatcct |
| 14521 | gcttgtgtag gcaatagtga taaaaacaca acaacaaaga gaggtaaaga gcactttcct |
| 14581 | ttgatataag taaagggcac gacttattgc acatatatat ataggtattc aactgagatt |
| 14641 | caacatgttt ctctcattga aacagcaagc tctccaggcc ttcatgttcc cagtgaggta |
| 14701 | ggtacccttc tgatgattat actcaccctc cctcattgca aagctcccgt tgttattgtc |
| 14761 | ttggctctgg attccctcaa aaatagacta tgaaacaaat atctggggtc agatacttta |
| 14821 | atcagaaatt gagtgagaaa gcacagaagt ggagaaaatg aaacagaaca cgaagccagt |
| 14881 | gtgaatgagt agttactgct atgtgctcag taatgacgga ggtatggaga ttgtctcaaa |
| 14941 | ataactttac aaagagatgg ggatgctgga atccccatct cttattgctt aaggattacc |
| 15001 | ttagaatcat taactctcca cccctaactc cttctttgtt cctatgtgtg gttgagaagc |
| 15061 | actggttagc ctcaagaagc ttgcaggcag gtccacaaat cagaaagaca ggcatgatgt |
| 15121 | ggggagctct cagttagctg gaaacaggtg aatttcaggt gaacacattg agtccaggac |
| 15181 | atagaagaca agtcatcaac aatatctgct atagccagtt ttctttttct ataacacata |
| 15241 | tactttttat tgggggtccc caagtccccc tttggtttaa tgattcacat aactcaagaa |
| 15301 | agctgatttt ttttgtggtt atagtttcta acagtgaaag aaaccagatt aaaataatca |
| 15361 | gaagcataaa agcacataaa gttgagtcca ggacaaacca gatatgagct tacaggtgtc |
| 15421 | ctttcatagt ggggacttca cactgactaa ttttctttac aatcatgtga gacaacatgt |
| 15481 | gtgaacttgt tgccaactag ggaagctcag tcagtcttga gtccagggtt tttattagga |
| 15541 | ttccaccaca tatgcatcga gcatcctgtg actgaactta gctacttagt tcccaacctc |
| 15601 | cctatgccct gagaggtcat attaatatgg cattacacaa agtcataggc atacagaaac |
| 15661 | aggtgctcac aagaaataac gttgttagca tcagctattt ggtatgacct acgttttcaa |
| 15721 | gtatacaaag actctcatca ggcagcatat accaagggct cataggttgt catctcccag |
| 15781 | gaggttgtca agggccagtg ctgaagacct ttggaatgcg caaggttttg gaaagccatg |
| 15841 | tctgcagaat taaccattac acaccttcca agaatttttt ttctctttaa aaatgttttt |
| 15901 | tgatctttga caatgtacca accaagactg agtaattagt aacagcagtg tactcctgag |
| 15961 | tacttgcacc tgcaaggaga aaaaggacag atgcacttac ataggacaga tgcaaataga |
| 16021 | caccactatg acaagtaaag ctggaataat caataaattc ctaaagacaa agtggggctg |
| 16081 | gtcagattgg gagaccgctg acagctgcag aagttgggaa agatccatca tcttgaaaac |
| 16141 | tttttcccca caaacccact gcgatctctc aagcaattgg taaggaatcc aagagagtct |
| 16201 | gtatatgaca cagatcaggg agagcagaac acttgggagg tgaccaggtc ttgggggctg |
| 16261 | agcccttatg aatgggatta gtgcctttat aaaagaagct caatggagtt cttgtgtgcc |
| 16321 | ttccactatg tgaggacata gaaagaaggc accatctatg aaccaagaaa tgggctctca |
| 16381 | tcaacactga atttgtgagc atctttacct gagatctttc agcctcaaga agtgtgaaaa |
| 16441 | aagaaatatc tgttgctttt tagtcacccg gtttatgtta ttttgttata agagtccaaa |
| 16501 | tagaccaaga tattccattc cacttaatat gtaggggaag acaacaaaaa ctgccacact |
| 16561 | tagaatactc ctgatactgg aagtatgaaa acaggaaaaa caaaacaaaa ctgctcttga |
| 16621 | aggtgaagga ggaatatcac tgagctcacc aacacagcca ggaaaagaac agaagtgtga |
| 16681 | gaaggctaca ttcctgagac cctgagaaaa agtacctgca taagactgag atgaaattac |
| 16741 | ctaccctagt tataattgaa atcccaaaaa gaggaaaaaa taatggagca aaagaaatat |
| 16801 | ttttcaaaat aactgccaaa aatattctaa aagaagtgac agaaaatcaa acttcagata |
| 16861 | taggaaactc agagaatgtc aaatagaaca aaaataaata agaaatacat cttgaaaaat |
| 16921 | ctttaagaaa tcaactctaa attttatatc ttgctccaaa tatatagaga tataaatagg |
| 16981 | ttagcatgaa gatatggaga aagccatatc atggaaacac taaaataaag ctgtggaagg |
| 17041 | acattgatat tagacacaac aaagttcaga acaagaaata gtatcagaaa tgagagataa |
| 17101 | tagataatat aataattctc aagatgtgaa catcctacta attagggtat gcagctaaca |
| 17161 | acagaacctc caaatacatg aggtaaaaca ggaaagaaat caaaggtgaa ctagaaaaat |
| 17221 | ccaaaattat atttgcagac ttcaacactt ttgtcttagt aatggacaga ctaggcacaa |
| 17281 | actcagtaat cctatggaag ataagaacaa caatatcacc aacaagacac gcaatcttca |
| 17341 | atggcagaca cgctttcaag tgaaaaaaaa aacagtatgg catattttct aacaaaccca |
| 17401 | gaatttctaa tatttgcggt cttccttcca tcttcctttc tcttctcttc ttcttttcct |
| 17461 | ttcttttcct tttttctcct tccttcccct tattcttcct tccctcctcc ctcccttttt |
| 17521 | ttccttcttt tcttattctt tctttccttt tttcctccct tcctccctcc ttttcttcct |
| 17581 | ccctcccttc ctttttcctt ccttcctccc tattttgttt gccttcctcc cttttaccat |
| 17641 | tctctcttcc tttccttcct ccttccttcc tcactttttc tctttctttc ttgagttctt |
| 17701 | gctttctttt ttctcccttc ctccctccct tctctcattt cctccttttc tttcttcttt |
| 17761 | ctttccttct tttttctttc tctttacaac tcatattatt taaaaaaaat taagacaggc |
| 17821 | agaaaaataa agaacacttt aatctgcagg taaatagatt atgtctgctg tagacaaaat |
| 17881 | aatggcctcc caaaaatgtt catgtcctaa ttcccggagt ctaacataca aatatgttat |
| 17941 | gttgcatggc agtgggaaat tagatttcaa gtgaaattaa ggttccaaag gcagcggggg |
| 18001 | caaaaagcct aggcggaggg ggcaaaaagc cgcggcggcg ggggcaaaaa ggagcggggg |
| 18061 | caaaaacccc acaaaaaccc gcggcgacgg ggggaaaaaa ccgcggcggc aaaaagcctc |
| 18121 | agcggcagga gcaaaaagcc gccgcagtgg ggataaaaag ctgcggtgga aaaaacccgt |
| 18181 | ggcggcaggg gcaaaaagcc gtggcttcgg gggcaaaaag ccacagcagc aaaaaccctc |
| 18241 | agcggcgggg gcaaaaagct gtaaaaagcc gcggcggcgg gggcaaaaaa ccgcggcgcc |
| 18301 | aagggcaaaa agccgcggtg acggggcaaa atgccgcggt ggcgggggca aaaagccgcg |
| 18361 | gcgggaaaaa accgcagcgg cgggggcaaa aagctgccgc agcggtgacg aaaagccaca |
| 18421 | ggggcggggg caaaaagccg caaaaagccc cgacggcggg tcaaagagcc gcaaaaagcc |
| 18481 | ccagcgtcgg gggcaaaaag ccgcggctgc cggggcaaaa agcagcggag gcaaaaaccc |
| 18541 | gcggcggtgg ctgcaaaaag cagcggcggc aaaaagccgg ggcggcgggg gcaaaatagt |
| 18601 | ggaaatgggg tagaaggcca gcacagcttg gcattcctgg actgtgatgt ggaaggaaaa |
| 18661 | gtgcagcgca agacaaagat gtaagtaggc ttgactcagt gcagctaaga actcagatgt |
| 18721 | tatcttgatg ttaactatca gctgattttt tgtattttag tagagaaggg gttttaccac |
| 18781 | attggccagg attgtctgga tctcctgagc tcatgatcca cacacctcag cctcccaaag |
| 18841 | tgatgggatt agaggcatga gccacaaagt gctcaaaaaa tctattaatt aaaaaagtgt |
| 18901 | atgtagccgt cttcaatcta ccatgtccat tagcagataa atactacaag caaaataaca |
| 18961 | acaatgaaat aaacatagac ttagagtaga tactctgatt taataaaaat ttgaaagtag |
| 19021 | accaaattac gattaaaaaa actctgttac tattgaggat gagggttagt gtttggaaag |
| 19081 | gggcaggaga agtatcacta tttttagtaa tgttctattt tcatacatgg ctataagcaa |
| 19141 | atatatgtgt ttcattaatc aaggtatcca tatttaatca ttgtactttt ctgcatgtat |
| 19201 | gatatatgtc aataaaatgt cttaaattat atacagcaaa aatagacaaa accacaagaa |
| 19261 | gacatacatg aatgttaaac ctagagagaa atttgaatat aagtaagtct ctgaatgact |
| 19321 | gctagaacaa accaaaaaat aatcaggatg gagaggtttg gaacagcatg attagcaaaa |
| 19381 | ttgacatatc tgtcttttaa tataggcaga aacatagatt aaaaaaggac ttgtctcaga |
| 19441 | gtatgatttc tgaaaatagt ggaatcaagt ttgaatctag taagtacata taaataaatg |
| 19501 | tcttaaaact cttatgttag ctaattaaga aattattgta atagatatta gaaaatattt |
| 19561 | taatacattg agtggatttc acacgctaag gaaatgatct tacttgcatt tgatagttca |
| 19621 | attacataca tatataccta taggtagttt aaaatatttc taataacctt atatactttt |
| 19681 | aaaaagcatt gatatctgtt tgcactatct ggtctataga atacacatac caaacatgat |
| 19741 | tatagctctt ctgctataaa cttcaaatgt ctaattaata caaaaatcta gaatgagaag |
| 19801 | agttctttgc aatttttttt tttttttttt ttttaccaaa tagaatatag gaaggatagc |
| 19861 | tgcaaatata cctgacacac ttatctgtga gtatggtggt agccttttta ttttattttt |
| 19921 | gagagagggt ctcaatttgt cacccaagat ggagtgcagt catgtgatta gagctcactg |
| 19981 | aagcctttac atactgtgct caagcgattc tcccacctca gcctcctgag tagcagggac |
| 20041 | tgcaggtgca tgacaccata ctagctaatt tttgtaaaga tggggtttca ccatgttgcc |
| 20101 | ctggctgatc tccaactcct ggactcaaga gatctggcca ccttggcctc ccaaagtgct |
| 20161 | gggattatag ttttgaggca ccgcgatcag cccagcctta aaaaaggctg actagagatc |
| 20221 | tttatctatg tatatctata tctatctata aaataaacgt gtttattata taaaaatata |
| 20281 | tattattaat attatataaa aatttttttt caaggtagaa atatataaag agggtgcatg |
| 20341 | tagagcctgg gtcattgtgt agtgaagctc aaggcctctg aagaaatgcc ccttgcctct |
| 20401 | tttgtctggg ctagaatcca agaagggaaa gcagcagatg cactggttcc caggttcttg |
| 20461 | gcatcctaca gagagaaaca tgtttgagct aggatagcgt taaacaccct tcttctcact |
| 20521 | ctcctgtttt atgtagtgag cagagactag cttcatgaga acagactgtg acagtcaagg |
| 20581 | ctgtctgtta ttttgtgcag cattaattga gaaattctag cacctgaaga cctctgggcc |
| 20641 | atttgagggt aggtgcaggg aaggaaaggg aagtttgcat ccctcctgct gtggagagaa |
| 20701 | cccgtgggaa gcacagacct tgtcctaact aaaggcagac ccccttgcta accagcttct |
| 20761 | catcagccaa ccctgggtga gtttccatgt ctatttacta aataatcctt attgcttttc |
| 20821 | ttcatagggg caaagtatgg tttacaggga atattgttcc tttgaacacc cattgtgcaa |
| 20881 | actccttcct gttgtgggaa aacaggcttc catatgtgtc tttttgggaa acacattggc |
| 20941 | aatttctatg tttttactgc atctatttct tggatatggg aactgaatag tgcccatcaa |
| 21001 | aggctcacct gatgttggat attgatctga gagcacggaa ggacagaatt ctttctttgt |
| 21061 | tcctgggcag ccgtggttga gggatcattt tgtggcagct acagtggcaa tgatggaggc |
| 21121 | agaacggagg gctcagtacc aagacaagga gagacttggc ctcacaatgg cagcattgca |
| 21181 | ggggtgcgtt ctacagagca tttgctcaca tggttttggg cattgtctct aactacattg |
| 21241 | cttccccaat aggttgaccc attctaaata actccttttc tctttaaaac agaaaacttc |
| 21301 | atttgtatga cttgcaattg taaatgacac gaattggcca gttatcattc aaattccctg |
| 21361 | ttacttaatc ctgccttttt ctaacgtatg caactttccc ctaaaaaact ggacacttcg |
| 21421 | ttgcttattc attgtcttta cacattttaa aatgttgctt tatgccccca atccctaact |
| 21481 | acattttcga tgttttgcaa gtggagtcca tgtgttcttg atttacatga agctcaaaat |
| 21541 | aatggttata gtaactagta cttcataatt aagcaaaaag ctcttattga aaaatgacag |
| 21601 | aactatacat agagatgaca acatggagag atatttcctg agatcacaaa gttatggtat |
| 21661 | ggcagaacta gaaagttgag tagagactct gttcccaatc attatttcta ccaccagctt |
| 21721 | tctattttga tgttaatagt gttcttatgt gggaaaccct acatatttgc cagtgtttag |
| 21781 | ttcattgaca aatataaaga acttcaagaa cactctaatc ttttaaaaat aaaatatcta |
| 21841 | taattggcca tatgaaaaaa attggtactt gacatatact gagatcgttt tattttgtgc |
| 21901 | tagacaaatg aagtcataga acagaatgtg ctttaaatat tatgaatagt gcttgcgtgt |
| 21961 | gtgtgtgtgt gtgtgtgtgt gtgtgtgttt atagatgcat attaggctgc tgaaaagttt |
| 22021 | gactattctt tccaggagag agactgccaa cttttgaacc taattagtac aagtatattg |
| 22081 | cttcttcata tttttattaa ggcaaagaga gtctagttaa aaataattca acttattgtg |
| 22141 | gaaatgctat aaattgctgt gaagtgagtt gctggctatg gcttgtcaga gcgaatatat |
| 22201 | tgtacaaatc ttaggggaga attagtgctt atgcattaaa atcaaatcat cttgcagcac |
| 22261 | actgagaaaa aggttagatt tttaaaataa tttcagagtc atgaaaagag caaatatgct |
| 22321 | caacaaagag cctagcaacc ctcaatgacc aattcccctt ttatatagtt tggtatctga |
| 22381 | attagaatcc cagaatctac aaattcctct gggtgtgggt gctgcatttt gaggatttta |
| 22441 | taacactgcc atcaccaagc tctcttttga tattcaattt aaggagataa tttacgggca |
| 22501 | accagagagc atacaccaaa gtaggtatct atctagatag acagatacat ctccatatca |
| 22561 | ttgacaggat actttctggc cgagcgtgag tacaacctat gggtgtggtt ggagagaaca |
| 22621 | tgtgttccac ctgaatggca gatcaagatt attccttctc atctgctgca atggctcaat |
| 22681 | gtgttaagga gaggagtgag acagcaagaa ctgcattcat tcagtcatac agaccaaaag |
| 22741 | gaggaatgtc gcccagccct ctaaactgac ccagaaccca gctcatgtct caactgctac |
| 22801 | ctctactact tagaaagaag taactccgct aaagcagggt tctggacaaa tatattttta |
| 22861 | ttgatcatat acaaatagat gaagatggac ttggatgtta agaaaaataa tactatacaa |
| 22921 | aatcaagagt agacagtcac ccctagactt aaattaagag tgtgtacatt agataattta |
| 22981 | atccaatgta tcaggtaaaa acttgaacaa acgttttggc ctcttccata aaattcaggg |
| 23041 | aagcatgtcc tccacaaaac agaatcaaaa tataaatgaa agactggctt aagatgaaag |
| 23101 | gaaaccttat aaatgaaaag aagccagatg agaggcactt aactgagaat gaaaaaaaac |
| 23161 | tgagtggaca aaataattat gagaagatga atcttcaaat cagaaagagg gaaaaaagct |
| 23221 | catttgatac tatgggaact caaaagagag tgaacacaaa tgtgaaaatt ccaagagtac |
| 23281 | agaaaagtag cataactaaa ttaagagcat gagaaaatgt atacaattct gagtaataag |
| 23341 | aacagaaatc aaaagttagt attgtatgtt ttagtagagc aacactgaag acgaatgaaa |
| 23401 | acaagaaata atattaaata tgaacatatg gagaacagaa caatattttt aaaattttta |
| 23461 | gtttctaagt ttacctgaaa ttttaatttt ggtttcttat gtaataccag agttattagg |
| 23521 | aagatattag ctaataacac tattttcagt ggtattttaa gtatttgtcc tagaaaaatt |
| 23581 | tctatttttt aaaaatgtat atttaaaaat acattaaatg tgtatataca tcaatcatat |
| 23641 | gtatcgattt ctgttttttt tgaattgcaa atgaaatttg tatttttgtg ttcctggaat |
| 23701 | aaaataaact tgaatggatt gtgatatatt attcatgctg taattcaatg tatttgaatt |
| 23761 | ctttaagaat gttacattta tagttaacag atattgacct ataaattttc tttcatataa |
| 23821 | taatgctgtg agacaatata agaagaatta aaatttaaat tcatgtattc ctactttttc |
| 23881 | ctctgttctc taactgtaat atattttaat tacagatgga ggaacagata gatgttagat |
| 23941 | aaataggtat ataatagatc atccaaaatt cttattgttt tatgtagtca gtatttacct |
| 24001 | ctgtttttct acatgtttat ccttccaatt tagttcatta ctttctgcac ctttgatgtc |
| 24061 | atatacataa acaggaaata acacatggtg gccgggatgt agagagagcc acaggacttg |
| 24121 | tgaatacaat ccacaggcaa ggatgtggcg attccttttg cagtattgga gggaatgcca |
| 24181 | aacgctatgt ttgctgtgga aaagagtatg gtagttcctc aaaacatcaa aatggtattg |
| 24241 | ccatatgatt cagcagcgcc acatctcagg atagcaaaag aattgaaagc agagtcttga |
| 24301 | aaaaatattt gcacatccat gcttgcagca gcgttattgg caatagctaa aacgtagaag |
| 24361 | aaattgaagt gtccaacaac agatgaatgg ataagcacaa tatgatatat acacacaatg |
| 24421 | gaatattatt cagccttaaa catgagggaa atattctgac atatgttgca acttggatga |
| 24481 | aacttgagga tattatgcca agtgaaataa gttagtcagt gaaggacaaa tacagtataa |
| 24541 | ttccatttgt attaaagtgg acagaatcat agagatagta caatgatggt tgccagaagc |
| 24601 | tgtggggagg aagaaatggg gaagtattgt ttaatgggta tagagtttca gttctacaag |
| 24661 | atgaaatgag ttatggagat ggatggtagg gatggctgca caatgttatg actatattta |
| 24721 | gtaccactga actgtacact taaaatcgtt aacagagtac attttatttt atgtgtattt |
| 24781 | taccacaata aaaaaataaa ataccttagg aacattttcc tgaaagagtc cacataaaat |
| 24841 | tcattttaat gcatgtgttt atgcatagct ttctattttt ctcttttcta tttatattcc |
| 24901 | aaattagaat ataatgctaa tcaagcatag tggctgtgtt tcttgcttcg tctagtctgc |
| 24961 | aggtagcata caaatgtaat aaactactta ttcatgtcac atctatttat tttctgcctt |
| 25021 | ataccaagct tgtgggattc tcttaaatac aacattttta tacttacacc taggcaatac |
| 25081 | ccattagcat cgccttccta aatcagggga aattgagcct ctgtaaggtg gagtaactta |
| 25141 | ctaagataca aaactcagca ttaaagtctg tatacttcaa tatcctgccc tcttctcatt |
| 25201 | tgtctttact gccttttatg tatgtgttag atgttcaata aattctcttt tttaaactga |
| 25261 | atttaagccg tggagcagtg ttttgttgaa caataaatat gatataggac actcttcctc |
| 25321 | cctttcattt atgatccagt tcatgaaaaa gagaaattct ttcattgtgc tagaagctta |
| 25381 | aaataatgaa aatgccactt tctacattaa acagaaactg aagggaatca aggtgaattg |
| 25441 | cacgagacat agaaaacaag tgggaaagaa atctagtata atttgccctt tttgtacctt |
| 25501 | tattatttgg cgtttgagta aatgtttccc ccaaatatct tcccatctta attcatgtct |
| 25561 | atgaagtaga catttatgtc tcaccttgtc aagaagggca aactctaaca taaacatttc |
| 25621 | ccaaaaatgc tttctgctaa aacgtaagct cagtctggct agaaatgaag ctcacttcat |
| 25681 | aaagattaat tggtagctaa tctttgcatg ctgttctctg aacctgagtg aaagctgtcc |
| 25741 | atcaggcata cagggaatga cggaaaaggt gacaacagaa gatgaatgct atgtcactaa |
| 25801 | ccttcaaaga tgacctgcct tttctttcaa attcttgata tcttaagact tcattatttc |
| 25861 | atctctcttt gcccttggtt caacattgtg ctataccaaa actcatgtaa aacaatgatc |
| 25921 | taatgtaata aaaatggcat ttttctttca tgtagatgca agctaactgg catttttaca |
| 25981 | atcaacgtat ttcctttgtc aatttttcat tctgtattgg aagtaattga taggtatttc |
| 26041 | tgaagggatg aaggtgtttc tgtgttcatt gtgatccaaa ctatttttag acctaggggc |
| 26101 | atttgtaaaa caatttgtgc cagctgacca aggaccactg tggcagaaag cagcaaactt |
| 26161 | gcataagatg tcactgcctc ataagttggc tttgaaaact aggggcttac tctatagtct |
| 26221 | tatgaatcaa agacattgat agatgtagta taagattaca atcatatttt ccttttgaca |
| 26281 | gtcacattat aaagcatgat gtattgcaat taatctcaat tagctgatca caattaaaat |
| 26341 | taatgtttat tattgctgat aaacaatcat gaatctcctg ttctcaaatg tgcaagtaat |
| 26401 | tcttgtaatt ttaatacaaa tttgcatatt attactaatt gatttaatct cattggattt |
| 26461 | ggttcatgga tccaatttat taaaatattg gtaatgggat aatgatttgt ctctccattt |
| 26521 | catgtacgct aaaagccaca attcttacaa tggtctgcaa gcccatcatg atccgctgca |
| 26581 | ggttaaccac gaaaattctt ttatttcttc acccttgatc ttaccagtgg tcctggccac |
| 26641 | ctcactgtcc tctggacatg catgtttgct gctgtcttat gaccaagact ctagttaatt |
| 26701 | tcttggcttg gaaagatagc cctccatata tccattgatc agctcactca acttcctcaa |
| 26761 | gtctttactg aaacctcaca ttctcgatga gacctattcg gtatttcaaa ctgcctccca |
| 26821 | gctgcaacat tcccaaaccc cgtactcttc tgtgtatttt tgaaaggatt tattgagata |
| 26881 | taatttacat agtgtagagt gcacacatta atgtctacaa gtcagtggct tttagtatat |
| 26941 | gcacaggtaa gtggagccat catcacaatg aattttagag cattttcttc acttcaaaaa |
| 27001 | gaaaccccac cttctctagc tgttaacctc ctatgcaccc atcccctact caatcctaag |
| 27061 | caaccacaaa tctgttttgt ctctatagat tttcctattc tattttcatc taaatagaat |
| 27121 | catacaatag gtggcctttt gtgcctggct tctttcagtt ggcataatgc tatcaaggtt |
| 27181 | catatatgta ttggtacttt atttcttttt ataactgtag aacattcaat ttcatggata |
| 27241 | taaaattttg tttatccaat aatattttta ttgacatttg agttgtgttc agcctttggc |
| 27301 | tattttaaat actgctgcta aaaatacttg tgtacaattt gtgtttgaac acctctttcc |
| 27361 | aataatctgg gtgtatacct aggaatacat ttctgggtca tatgacaatt ctatgtttaa |
| 27421 | tatatttaga agccatcaaa ctgttttcca aagtggccag ttctagccat agagtatcta |
| 27481 | actgtggttt tgatttgtag ttgcctgatg agtgatgcta ttgagtatat ttttatggga |
| 27541 | ttattggccg ttcgtgtaac ttcttgggaa acacatctat tcctatcatt tatcagtttt |
| 27601 | gagttgggat atttgttact gagttaaaac aatttttcta tattcaagat acatatatat |
| 27661 | atatacagac atatagacac gtgtttttca aatatcttct cacaattttt gagctgcctt |
| 27721 | ttgacttgct tggttgtcct ttgaaacacc aatgtcttta atttttaaga aattttaaat |
| 27781 | atctaatttt tattttgttg ctcatgtttt tggtgttaca gctatttctt tgctagatcc |
| 27841 | aaaatcctga agattttccc atatgcttta ttctagctct tgcatgtatg tctttaattc |
| 27901 | atttgagtta atatttttgt atgctttggg gtaagggttc gaatttatta ttttgcaagt |
| 27961 | ggtgatccat gtgtacgttg ttgacccagc ttgttcaaga ctgtctcttc ctcattgaat |
| 28021 | tgcacatggc accactgtaa gaatccattg actatagata catagtttta tatatggact |
| 28081 | ctccattctc ttccatcaat ctatttttcc ttcatcagta ttgtgttgtc ttgattactg |
| 28141 | atgctttgca gtaaggtgtg cagcacgggg gtgtgaatta tcctattatg ttttcttttt |
| 28201 | tcaaggttat tttggctatt ttgagtccct tgcgattcca tgtgtatttt agaatcagct |
| 28261 | tgtcagtttc tagacagaag tctgttggga tacttttagg gatttcatca aatctgtagt |
| 28321 | tcaaattgta aagtactaca atattaaatc ttccaattca tggctataag acatttgcta |
| 28381 | attatttaga tcttctttaa acaataattt ttaattttca gagtaaaata ttgtatcaca |
| 28441 | ttttccaaat taattattat ttcttttttg acgctattgt aaattgaaat gttttcttag |
| 28501 | tttcattttg gggttttcat tgtagatgtg tgcaattgat ttttctatat ttatcttgta |
| 28561 | tgctgtaata ttgctgaaat aatttactag ttctgtcgtt cagtggattc cttaaaattt |
| 28621 | tctatataca agaatgttat ttgcaaataa agttttattt ctttctattc aatatgggtg |
| 28681 | actcatttct ttagttgccg atttgccatg cataaaatct ttagtataat gttgactaga |
| 28741 | agaggtcaaa gtatatatcc tattcttatc tctgaccata gcgggaaagt atcctttctt |
| 28801 | ttaccattaa gttgcatgtt tgctgttggc ttttcacagg tgccatgtat ctggcgtaga |
| 28861 | aagttctcta ttcctggttc attgagtttt tatttttatt tttaatcatt aaagaatttg |
| 28921 | gattttgtta aatgtctttt ctgaatctat cgagatgatc atgcaattct cgtttcttat |
| 28981 | tctatggata agatatatta ccttaatgga ttttgggctg tttaaccaac ctgggattac |
| 29041 | ttgtataaat ttcactttgt catagtttat aattctttta tatgttgcta gatatgattt |
| 29101 | gttagtattt tttaaggaat tttgcattta tacttatagt agttttattt ttctatgcta |
| 29161 | tttggactaa tttttgtatc aaggtaacac tggccccaca gaataaattg ggaagtgaat |
| 29221 | atttctcttt tttaaaaaag ctagtcaaga attaatatca attattcaat actaacaaat |
| 29281 | attgttataa attattaatt tctctaattt ttattttctt ccttctgctt gctttagttt |
| 29341 | tgctattttt tccagtgcct taatgtggaa ggtcatctta tctcatcctt tcatttgtct |
| 29401 | tttcattttc taaatagtgt ctttttagca tcaggtgagc tccccaggtt ggtagtactc |
| 29461 | catgtttatt gctgtacaac aatgacaggt aatatgtcct gaagacaatg gaaacttaac |
| 29521 | attcaaaatc ctcctagatt ccaccttata tgatatgtct cttcctttaa ttggtcctaa |
| 29581 | tttctaccct ttctctatta taaaccatga gtacaatggc attcaatgag ttctgtgagt |
| 29641 | ctttctagta aattcttgaa tctgagggtg ttcaggggaa acccctgaaa tggcagttgg |
| 29701 | tgtcagaagt gagaatcgtc ttatatggcc tcttcctttg aactttgcag ctggacccaa |
| 29761 | actctgcata atttgggcca gaagtctcgt gttgactttg cagcctaaag taccttgtag |
| 29821 | tttgtctaac cctcaataaa tttgctttca tcaaatattg tatttgttac cgcaaaatta |
| 29881 | ccatcatgtt ttttttctcc aaataactaa cattgggaga aatagccagc tgaatctgta |
| 29941 | actcaacaga aacaagtgat ccatatacca tataccatgt aagtggccat tttgcctcct |
| 30001 | tccaccacat cttagcaacc tcaaccattg ccatgagcca ctgtaggcct accggctaca |
| 30061 | aacaaacaag tatcttttaa aaacacttca tactcccatt tgataaattt cccagcaaag |
| 30121 | agatgtctac tttaactcta tgcaagtggc tcatattcac gaagtctgga gatattattc |
| 30181 | atgtagtgtg agaaaatcat cccagcgatg ccagcacatt ctccttccca taatctgctt |
| 30241 | agtttgcaaa catattcagg ccataggtga gagatttgta tttcacagta caacaatttt |
| 30301 | atggagggca ttgaaactta gattgagcat tttagtacag tcacacatca ctgaatgata |
| 30361 | gggatacgtt ctaacagatg catccatagg caatttcatc attttgcaaa cgtcagagag |
| 30421 | aatattacaa acacctagat tgtacagcct accacgtctg ggttatacgg tatagcctct |
| 30481 | ctctcctagg ctacaaacct gtgtactaca ttactgtact gaatactgca ggcaataaga |
| 30541 | acacagcggt aagagtttat gtatctaaac atacttaaaa catagaaaag catgtaaaaa |
| 30601 | tatgtattat aatctcatag gcccactttt gtatatgtaa tccatctttg actgaaatgt |
| 30661 | tattatgcat gacatgactc tatgacaaaa ataaaataac acactgtaaa aaattacata |
| 30721 | tgtatcaaac atattaatat aaaaataaaa atattcagtg taagaatttg taatgatcac |
| 30781 | aaaatgttca cagcttatat tttagtacag tttcaaatgc ctagtgcaat tactatttat |
| 30841 | ttgtgtattt taaacatgta tataataaat atttttcagg ttcaacaatg tatatcaatc |
| 30901 | caactggctt ttataaatat tagttaaaat caattagtaa attcatatat atatacacac |
| 30961 | acgtgtatca gtgtgtgtgc atgtatgtgt gtgtaaatgt aattgtatgt gtgtgtaaat |
| 31021 | gtaactggat gcatcctaat atttagcctt acctacaaga tttccaagat tcatttatta |
| 31081 | tctttagatg atgtgcactt aaagatttgc caaataaaac tgtaaccgtg gaaaatatca |
| 31141 | agatgttatt aaattcatct tgtgcacgta attgtttctc tacatttatg tttcttgcac |
| 31201 | aacttgcagt aatgctcatg cacaaaataa ttttctaaat aaaaaataaa aacattttct |
| 31261 | cagtcattaa ttcttaataa ttatttctct ctgataatta ttgtgaatta attcttaatt |
| 31321 | cttaattata gaataatgtt gcctttcaga gttctgaatc ttttgcatgt tgtatacatt |
| 31381 | tcactaacta gaacaacttc tggaatattg gcattaatta atgtcactca gcaattattg |
| 31441 | atttcaaaga aattaaatac cattcatatt ctgaatcaca agggtgcttt ggcatctaat |
| 31501 | ttaatcaagc tctttgtatc atcatctaca ctttaattac ttaacaaaca tttctctgtg |
| 31561 | tgagaaagat tgagcaggtt attgtgcttt gttaagatgc aacttttgct taatctagag |
| 31621 | ataggcaatg ctctctataa gggacaaaga gaaaaatgaa agagcaatag agacgtgaca |
| 31681 | ggcatggaaa aagacaatac atttataaaa caaataggac cacagatgac aataatgggg |
| 31741 | atcaaatctt gagatactga ctcagtttat aaccgcactg tataatagag caaatcactt |
| 31801 | gttaattttt ttaaaaatgg aatttaattt aagatgaata cagtgtttta aacaagacag |
| 31861 | gtcatcttaa aataaaatag tggaataaag tgataaaacc aatgtaaaaa tcataaacaa |
| 31921 | tttataaaga atttttgtca tgtaatttaa tatttttatt taaaaatcac caaaatcaga |
| 31981 | ataattttat cttaattaac ataatcatca gaagttaact aatttttact ttataatact |
| 32041 | acgtttaaaa atctttatat ttttaatcat atatgcttat atataaaata gacataggat |
| 32101 | atatatttaa tgttcccaat attatattgc aattgttcct atggatgtgg tttttcaata |
| 32161 | gaattaagta cttttaaaaa gtttcaatct caatgatata tatgtttgat ttttctttga |
| 32221 | caaagcatac atatattgat aggtaataat atgaaaatct tctaaagaca ttacaggaac |
| 32281 | atgaaaatgt aattaaatac tcactaattt gtaatgtttt atgtaagcgg aacacattta |
| 32341 | actgaaaatt gcttttatat aataaacgag actagaaaca ttttaactaa cggagtaagt |
| 32401 | cttcaaattg ataatctgaa ctatacaaga ggagaaactt caggcactca aatatttgaa |
| 32461 | atgatacaaa atatttatat aaactattat ttaacaattt ctgtttgtag agtgctataa |
| 32521 | tcaatataaa tgacatctca agtctttcta tcgctttgac cacatttacc tcctaatttt |
| 32581 | aattactaat atgttggagc aatgcataca actagattct gatcttcctt tttaatgagt |
| 32641 | aaaaaatatg tcctttgaga cagcattaaa gaaagagcac cttgtataaa ttcaatgcca |
| 32701 | agagacaaga tattcttgat tctgaagtct tgttctttta tacaacaatg taattaataa |
| 32761 | taagaagaaa agcaggacat agatgtggag tctattttaa tcaaaaattg tctatagatt |
| 32821 | ttgatgataa aatttaaaaa tctactatat ttagtcagtt acaaaaaact aggttgtggg |
| 32881 | aacatatttg gtcaataaaa cacccctacc aaatgctaac aagaaaaagt tatgtaccac |
| 32941 | ctttcttctc tgcagatggc ctgagatggg ttaatttgaa agaatgtttc caaacctgag |
| 33001 | gtgacccctg agaacagcat aatccactgc tgtctcccac attcagtttc tcagtttgtg |
| 33061 | ctcttttaat cttgggggga gggaagccag tcctttaaag caatcttcag catgatggca |
| 33121 | gagccaagga gtgtggacag gtggcacggt gtctgactta gttccagcag ccacttgggt |
| 33181 | ttctctgggt tttctctgcc ctagggatag caccactatt gaaaacatgt ctttgtgaca |
| 33241 | ttctctatgc caggaactcc caacacattt tccttgaaac tgatgaaatg aataaaaata |
| 33301 | aaccaagagg tgtgctgttt gtttctgctt cctcctttct gcagcccttc ttgatcatct |
| 33361 | aatattttta aatacattgt cgatcaccaa aaggagcata aggggtatat tgatttgtag |
| 33421 | ctgatgtatt aatagccgag cccctattcc ttacctgtag ctgctgggaa gaaaaccatt |
| 33481 | cttaacactc tacaaggtct catctccaga atttgcacct gtttctagct gaggactttc |
| 33541 | tctagcagca cgggagcttg atactgggca tgaagtggga agaaaaggtg agggttacta |
| 33601 | agaagaatct ccctggattc agtgatgtaa ttctgaggca tgttccacat agcttcccat |
| 33661 | agaattaagc ccagatatct aacacaggaa cttgcctgtt aacacgtgtg gtactggctt |
| 33721 | ttctatcttt cctgttttat tttattctct cttccttgtc tcactttcgc tgtgtcctca |
| 33781 | ctcctgcttt aagaataccc aaacaaatac attcatttat ttttttagac tctcagaaca |
| 33841 | cagttgatag ttgaacttgt aacctatgat aatcagcttg gatgctatac tgaaaggaag |
| 33901 | atggtgaact caccatgtct aattaagata aaatttaaaa aatatattga ttcatgtcca |
| 33961 | aagatttaaa aaacctaagc ggcagtgtca caatttcttc tttttagttt acatggtttc |
| 34021 | ttaaatgcct acaattattt taaaggaagc cttgggttta gagaaaattg agacatacgg |
| 34081 | aataaattac taacccattt ctccttgaaa tccattagat gctgatgatt tttcacatat |
| 34141 | acttctgaat tgaaaagcta gttgcgaatt atttttataa gcatatcctt atgtaatatt |
| 34201 | ttgtttttaa cagtaagttg aagatttaaa gattaaatta ttctatccag agaataattt |
| 34261 | aatctttaaa tcttcaattt actgttaaaa acaaaatatt acataagatt ttaaaacagg |
| 34321 | tcccatattc ttttgagtca attagaatat gtttgtatca gtctgtctac agttttacac |
| 34381 | ctgtcaaaat gtacttgaac tacaagtacc ttgaacaatt ttcacattta ttattcctct |
| 34441 | gaaaatgatt aaaagaatta gagtgaaatt ctgattggca taatttggga gagaaattat |
| 34501 | tccttggaga tcaacctctg ccaagatagt ttataatgac attgagactt tttgatttac |
| 34561 | aaagtttgtt atataaaaaa tactaagacg atgacagata atacacacac tttaattaaa |
| 34621 | attgtactaa aattaaatgt ctaaataaat tagaagggta cgtggtacat ctaattgtat |
| 34681 | gtttatatat ttcatttgtg tattttattc ctagggttgc ttttgcttta gtttgtaaca |
| 34741 | cgttcttatt tttatgataa tgtagtatat gctaaataaa gaaaaatcag gaaatagaaa |
| 34801 | atgaagaaga aaacattagc tattgtcaac caaataaaaa ttgtgcaatc tctaagcaca |
| 34861 | tgaactatgt attatttgta cagcatgtac aatgtttatg cttcacaggg tgaggtagag |
| 34921 | actgcaaaac attgaacctg ggacaaataa gaaagtaagg aaattttcac aatatattaa |
| 34981 | tattatagaa aatgttgaac ttaacagtta agatacaagt agtgaaaaat gatagtattt |
| 35041 | aaggagatct agaaaattta atctatatct gtaatgtgtg agaagtatta gaataatgct |
| 35101 | tgtatttctg gattggcatc gatttctatt gagactggaa acataataga aatgagggaa |
| 35161 | aaagaactta aattgtggat acttgagttt tatacctagg agtttgagaa atacattttg |
| 35221 | ttactatcaa agcagttggc acaagagtgt acaaaattcc ctaattgtgt caacgtggag |
| 35281 | aagacataga caaacagaga atagcaaaat agaaatagca aaaaagcaca aataaatttt |
| 35341 | acctgtattt ttacataaaa gccaattaga gtaggaaaac atgaaatttg tgtttaatca |
| 35401 | aaatttttct ctttcttata gtctagtgga ttatattact ggaaaaaaat tgaagcattg |
| 35461 | gtatgttcac aaaaaaaagt aaaatataag gtcaaaacca tgggaatgca gggagcagac |
| 35521 | aaaatatacc taaacaccga aactgatttt gccctacgga catgtaccaa aatgaatgag |
| 35581 | tgcagattcc tactgtcata catcacatag gacagtaaag aaatacatag tttttcctaa |
| 35641 | gatagggcat cacacaggag ctcctcccta aagctaggac caaagtttct atcctcagta |
| 35701 | taaagaagaa tcagaggtaa attagtccca tttcacattc cctggaaatg gcaaataaaa |
| 35761 | atgattgaga ttggacagat ttaaagaaat tcaatcatta atgatttaca gcaattaatt |
| 35821 | taaaaattgt ttaaatgtgc agtccaaaca tatgtccaaa cacctttagg ccaagaatta |
| 35881 | atataatgtg gtcccagaat ggtggtgcgt ttagtagact cacaaaaaaa ttcaaattct |
| 35941 | ctttggcaaa ttttcttctt actaatccgc aaaagtgcac aaaaataatt ttcagagaaa |
| 36001 | aataaatatt tgtcattcag aggcatctaa gtacacaagg aaatggtata ccaccgtttg |
| 36061 | aaaggaaagc agaaaaagag tacaaacaga tccacaaagg ttcattagta gaaatatcac |
| 36121 | tgttagatta taaagcacat ttgctttaaa aaattttttt taaaaaagaa tatattttag |
| 36181 | gagactaaaa aattgatgta gaaaatttgt aaagtagctt gtataaaaat atagtatttt |
| 36241 | aaattaaaaa ctcaaaaatg aactcatcag attagacatg gccatggtga gagttaataa |
| 36301 | atatttcaca atgcatcaca gaaaatttaa aaaaatgcaa aatgtggaca gaatcatgaa |
| 36361 | gagacaagga agataccgtg agaaagtgta gcatgtgttt agtgagtgtt ctcatagaag |
| 36421 | aagggaactg ggaagggaca atatgtgatg gtattttggc tgaaagttct ctagactttt |
| 36481 | gtaagacact aatccgcata ttgaaaaatt ccatgcatgc taagcaagct acaatggaga |
| 36541 | taaacctaca cctacgtatc tcctagagaa atagcaaaga atcaggaagg gaaaaatatt |
| 36601 | tcaattagca ctagaaaaat caaattacct ttaatcatat tgaaatcgga aagaatgaaa |
| 36661 | ggtaaaataa aaaatattat ttgttaataa taataatgcc attctgaaat tctcaaccaa |
| 36721 | gaaaaatatt catcaaccta tggctaaata acatatttag agagaaaaaa caaagcacca |
| 36781 | ccagcagaat tccactaaag aaactaaaaa gaaactctga aaacatgctt cagaaaggtt |
| 36841 | gaagttctga aatcaaagaa tgaatacaga gcaaaataaa catacaggta gatctaaata |
| 36901 | aaaaattagg tgttgaaaca aaaagatatt taaaattaga taagcactgc aatatgtatg |
| 36961 | ttaggaagca aattattagg gctgaagtat tcaatgaccc cttaattgtc tgacaagagc |
| 37021 | agaaaggtga gtatgacttt gcaacttttt tttttttttt tttttttttg aggaggagtc |
| 37081 | tcactctttc acccaggctg gaatgcggtg gtgccatctc cgctcactgc aacctctgcc |
| 37141 | tcccaggttc cagcaattct cctgcctcgg cctcctgagt agcggggatt acagccgcgt |
| 37201 | gccaccatgc ctggctaatt ttcgtatttt tagtagagac ggggtttcac catgttggtc |
| 37261 | aggctagttt ccaactcctg acctcgtgat ccacacgcct cggcctgccg aagtattgag |
| 37321 | attacaggcg tgagccactg cgcgcgaccg attttggaac tttaataaat tcagtggaca |
| 37381 | ttatgcattt ctctgttgta tctatgaaaa caataaaaat aaaagtcata atttaaaaac |
| 37441 | aagaagacag aaactgatag gagaaaatga cacattatat acatatatat atacaacaaa |
| 37501 | ttaataaaac aaattacgta taaatgatca aagattaact taaacctaag tagacaatgt |
| 37561 | ttttgttaaa atacaaagat tggcaaaatt aaaaaaatcc gtctctatca tagttacaag |
| 37621 | acaggcaact aatatataaa tttacagaaa ctttgaagtt caaacaatac agatactgtg |
| 37681 | tatatatgat atacatacaa acatactaca tgaatataat tttttaaaaa gttgctatgt |
| 37741 | agacaaaata gaatgtaagt tagaaacatt tattaaaata agttagtcta accagtgtga |
| 37801 | taaaagtttt aagttattaa gaagatgtga tgacttaaat gtgcattagc ctgatacata |
| 37861 | tatacatata tacacacaca ccacacacac acacacacac acacacgtat ttagatagtc |
| 37921 | aaattatata aagcaaaaat atcagaaagt aagtagaaat ggataagccc ccaaatcatt |
| 37981 | agacatttca aacacacatc tttcagtaat agataaaaga aaaaattaaa agagtaagtt |
| 38041 | ttaaaagaag ctagtggatt ttaaaaaggg caaatgttat ataaggaaca tgaatattat |
| 38101 | aattcatgtt attttcatgt tcatacagaa tacttacaaa aattaacatt ttctagacca |
| 38161 | taccacaaat tcaaacaatt ttcacggaaa taacgtgaca cagaatatat ttcctaaaca |
| 38221 | aacagcaatg aaggtagata tcaatacaaa aaagaaagct agaaacataa gtctaataat |
| 38281 | attggttgga agctgtttta attaatattg aaatatttta aaggtgaata gtcaacacaa |
| 38341 | atgaaccaaa cacttttgta aggccactaa gatgcatgtg taatgtgtaa tgcctccttt |
| 38401 | tataaggagt aaatctgtaa catcacctgg gctatttgac aactgcaaag tgaatgtgag |
| 38461 | aaggagagaa acagtgagag agagagagag ataaaaccag taaaataaac ataaagaatg |
| 38521 | aaggagatag ccgggcgcca tggctcatgc ctgtaatccc agcactttgg gaggccaagg |
| 38581 | caggtggatc acctgaggtc aggagttcga gaccagcctg gtccaacatg gtgaaaccca |
| 38641 | ctctctacta aatatacaaa aattagcctg gcatggtggc atgcacctgt aatcccagct |
| 38701 | actcagaggc tgaggtggga gaattgtttg aacgtggggg gtggaggttg cagtgagtag |
| 38761 | agatcacgcg actgcactcc agcttgggcg acagagcaag agtccgtgtc aaaaaaaaaa |
| 38821 | aaaaaaaaaa aaggaggaaa tagtacatga aaaagcagaa ttaaagcaac tgagtatata |
| 38881 | tttaaaaatg caacagctca ctttttcaga aaaatattaa aatattaaat ctaacaaata |
| 38941 | tctaggtaga ctgatggaga aaaatacaga aaatgcacaa aaaaccaatt acctggaatt |
| 39001 | caaaggttac aaaacgtaag cagttgtagg ttttaaataa gcaatgactt tgagttcaac |
| 39061 | catgatgggg tatattgaaa agaatctctc agaaaaaaag aaaaactgtt ataaagctat |
| 39121 | gtagaaaatg ttaagcacta ttaaagtctt ccaattctac cagttatgga gttattggtc |
| 39181 | ttggactaac tctcctgaaa agaaaaaaaa caaaacaaaa cctaaaaacc tggataaaat |
| 39241 | ggcctaccgt gggcactggc aatgcaacca agcaggtagg acatgggtgc tactttctct |
| 39301 | ttgtcagaac acaaagcatt catacactct tctcaccctc actctcacct tttaatctta |
| 39361 | gatctactat taaatgtatt caactactat caatcctttg gtcaaaattt cttttctcac |
| 39421 | attttgcttg atgcacttgg atagactgtt caagaaagtg tgagtactga attcctcaaa |
| 39481 | ctcttgcata tttaaaatta catttttgaa ccttgatgct tgaagtgtag cttgggtaac |
| 39541 | aggtgggctt taagccgatt ttggcatgca aggggttgag tttattaggc atcagcacct |
| 39601 | ctgaaaatcg tggggatgca ggcttaattt caacactatt ctaaatactt gaaagatata |
| 39661 | taattcctta ataaactcct ttgtctacaa atggttcaca ttaactcaat atccatgatt |
| 39721 | aaacatctat aaaatcaagg cactgttatt tagtggagac ttgctggcta ttctatgaga |
| 39781 | ggaggtattg ttattttaat ctcatcctct cataaaagtg tatcatatta ctcataacca |
| 39841 | gcccttcata ttctattcct attttggtat tttaaaataa gatatctttg aaactcttga |
| 39901 | attcaaatag agaatctgaa tagtttttaa aatgtcaatg aaatgccatt tcttcatgct |
| 39961 | tcaacaacta aaatttgact aaagtgcttc tcttcaaact ttctgtaaca ttttttaatc |
| 40021 | taaattctaa gaacgatcac aataggtttc aaccacaaat gtgagaatat tgtaaatgtt |
| 40081 | cgggtggaaa aatttttaaa ataattttgt agtaattttt gaatcatagt gacagtgcgc |
| 40141 | taaatttttt taagtcaaat attactgtag acatttaagt caagattcta agaaactgtt |
| 40201 | ctaaagtcca aaatttagtt tcatatacaa tgatattata tatatatttg catataaaat |
| 40261 | taatatatgt gagccatgtt tcaaatagtt gagagattat atcaaagatt cttgattata |
| 40321 | taaaatgcca attacttata ggcacacgtg ctttaaataa ttacaaaggc agctgtggtt |
| 40381 | gattctactc ttgctactgg catttatatg gacatactat tatggtctga agaatattta |
| 40441 | ggcaaattta tccctcatac gatcagaaga acaatgcacg atagtttata tctgaaagga |
| 40501 | aaaaaaatct ttatatggtt ctgaaagcct aaatcattaa caacttggat aataattagc |
| 40561 | ataaaaatac acaaacatgc cctcttccta gcggtaagta cagagtgaca acagaatcaa |
| 40621 | agcatgtggc tatgtgcatg tttatatttc aagacgcaga gcactctatt cctcttctct |
| 40681 | gccctttcta gatggcacaa ttcctcatga atctaagtgc tgtcataggg tggattaggg |
| 40741 | tgacctgcca tttgtatgca accgatctct attttggaag taattaatgt aaaaatatat |
| 40801 | ttttaaaaga taatttcaaa tttcagggca aactagcatg gtttcacccc tttcctttgt |
| 40861 | aacatttttc ctaaggttgg aaaagtaagg taggctttag tacgattttt aataacaagt |
| 40921 | tttcaaagtg agacgcaaaa tggtggcgcc aacacatttc aagtctgcta cattttgaat |
| 40981 | acacttattg gagaaaagac cttctcatca tttttctctt acaggaaagg aaataacatg |
| 41041 | tacagttgac ccttaagcaa caccgaggtt ggggtgctgg cccccctgca cagtagaaaa |
| 41101 | tccactaaaa ctttgactcc tccaaaactt aactactaat agcctactgt aagccttaca |
| 41161 | aataacacaa gcagtcaatt aacacatatt taatgttata tgtcttatat actgtattct |
| 41221 | taacaaacac gccagagaaa agaaaaagaa aatcataagg aaaatatatt tactagttat |
| 41281 | taaatggaag tagatgatca aacaggtctt catcctcatc cttttcatgg gcagggtgtg |
| 41341 | gaaaaggatg tagaattttt ggttttgcta agtggacccg cacaattcaa acccctgtgg |
| 41401 | tgcaaaggcc aactgtatag ccactgaata gcaatttatt tctagaaatt aacctcacta |
| 41461 | aaatattctt agaaagatgc caagaaaaaa atgaataagt atttttggta catctatttc |
| 41521 | atcatttcat ttcctcattt catcatttca tttcatcatt tcatttcatc atttcatcat |
| 41581 | ttcatcattt catttcatca tttcatctca acatttcatt tcatcatttc acttcatctc |
| 41641 | atcatttcat catttcattt catgatttca tctcatttca tcacttcatt tcttcatttc |
| 41701 | atttcatctg aacatttcat ttcatcattt cacttcatct catcttttca tcatttcatc |
| 41761 | tcatttcatc tcatcatttc atctcatctc atttcatctt ttcatctcgt catttcattt |
| 41821 | cgtcatttca tcttttcatc ccatttcatc atttcatcaa ttcaacattt caattatttc |
| 41881 | atcatttcat catttgactt catcatttca taacatttca tttcataatt tcatcttttc |
| 41941 | ttttcatcat ttcactttgt catttcattt tgtcatttca tttcctcatt tatcatttaa |
| 42001 | tttcatcctt tcatcatttt atctcatttc atcctttcat ttcattattt catcatttca |
| 42061 | tctcatcatt gcattttgtc atttcatttc atttcaacat ttcacttcat cttatcattt |
| 42121 | catctcatga tttcatttca tctcatcatt tcatttcgtc ttttcatctc atttcatcat |
| 42181 | ttcatctttt catctcattt catttgatca tttcatcaat tcatcatttc atcgtttcat |
| 42241 | tatttcatca tttaatcatt tcacttcatt tcatcatttc atcatttcat atcatttctt |
| 42301 | catttcatca tttgatcttt tcatttcatt tcaccatttc atgatttcat ttcatttcac |
| 42361 | ttcatcattt catttcattt caccatttca tcattccatc atttcatttc accatttcat |
| 42421 | ttcatcattt catttcatcc tttcattatt tcattgcatc atttcatctc ataatttcat |
| 42481 | tatttcactt catctcatca tttcatcatt tcatctcacg atttcatttc atctcatcat |
| 42541 | ttcatcacat ttcatctcat caattcatct tttcatctca tttcattatt tcatcctttc |
| 42601 | atctcatcat tcatcatttc attttatcaa ttcatcattt catttcatat ttcatcattt |
| 42661 | catttcattt catcatttca tcatttcact tcatcatttc attccatcat ttcacatttc |
| 42721 | ttcatttcat atcatttcat catttcactt catcatttca tttcctcatt tcatttcacc |
| 42781 | atttcatcaa ttcatttaat catttcatct catttcattt catcatttca cttcatctct |
| 42841 | tcatttcatc tcatgatttc atctcatcat ttcatctttt catctcattt catttaatca |
| 42901 | tttcgtttca tttcaccatt tcatctcgtc atttcatttc atttcatcaa ttcatcattt |
| 42961 | catttcatta tttcatcatt acacttcatc atttcatttc atcattgcat atcatttctt |
| 43021 | catttcatca tttcatcttt tcatttcacc atttcacttc atcatttcat ttcctcattt |
| 43081 | catttcacca tttcatttca ccatttcagt tcatcatttc atttcatcac tccatttcat |
| 43141 | catttcatca ttttctttca tctcatttca tcatttcttc atttcatcat tttatttcat |
| 43201 | catttcattt aatttcagtg atacatgtat tcaagtgcta atgtgatgcc caggagacac |
| 43261 | cctatttccc tttgtaaaac acctccttca acaaaaggca acctctcatg gctggctaag |
| 43321 | tctacaggga taccagcctc tcttcaacca cccaatttga tttagaacct caaacagcac |
| 43381 | ctcagtttca taaaaaccta aaacataaaa cacaacactt ggttgtaagt gagccaacag |
| 43441 | tttgtctctt tctctgccca aggcttaagg ccgtgtttcc ccaactacgt tcagtggaag |
| 43501 | aaaagatccc ctggacaaat aagtttgaga actgttgttg caggacttct cagaaccttt |
| 43561 | aaaacacaaa tcctcatccg gagggatctt caggagggag atgtctgatg cagcacaact |
| 43621 | ttctttcaca ggagcatcct gcagaataca gtatgagatg cagaaaggct gcaatgagtc |
| 43681 | tttttaatgg cccgggcctt ggtgggggtg gggtaggagc tctccagata gcatctaatg |
| 43741 | agtaggaaca ttcaggttgc tttttttttt ccttattggc aaacctgtgt gcacaccatg |
| 43801 | aatgaagctg atctccctta tccatatcaa aactaaaccc aaattaattg gctaaattgg |
| 43861 | gaatcaacac ctccaggagc tacgcagaag aaagccccac cacactttaa agtagcttac |
| 43921 | ctcatatttg acgaaagcaa aacacttatg accagtgtgc tgctaataca agtctacaga |
| 43981 | taacgctgca tgaaaaatta gttttcccaa tcgtagctgg catagtccac attttgcatt |
| 44041 | acactttccc cccttgtttt aaattttaaa cacaggtctt tttctcttct ttttttaaat |
| 44101 | tttaatttaa ttatacaaga cagagtctca gtatgttgcc caggctggtc ttgcactcct |
| 44161 | gagctcaagc gatacatccg tctccgcctc ccaaagtgct gagattacag gcctaagaca |
| 44221 | ctgtgcccgg ccttaaacac aaatcttaat tcattcttac agttatcctg aggttagaaa |
| 44281 | aatggaaggg gaagaaaaat ggcaagcagg taggttgact tcggcttcat tatttgaaag |
| 44341 | gacagtttgc tcagttaaaa cacactactg cccacaaagg ccaagacaac agaaaaatac |
| 44401 | agacatataa atagatttta tatgtgacag cagtttcaat ggagactttt tcaatgcaaa |
| 44461 | tgacaaacag ctgtgcttgg gaataaatga caaggaattt tttttatctc aacagctgtc |
| 44521 | ctgagagcac atctctacat ctctacctat attctggaat cagggagaaa gccaaaacgg |
| 44581 | atgacaagat actagatcag ccgtgtccaa cccttttagt acaaggactt ttccgcctat |
| 44641 | ctgtggtggt gggtatcatg aaaattatgc acaaaccttt tttttttttt tttaagctca |
| 44701 | tcagctgtcg ttagcattag tgtattttat gtgtggccca ggagcattct tcttccaata |
| 44761 | tgaccccgag aagccaaaag actggacacc tgtgcactag atcaaaaggc tactccttct |
| 44821 | ggaagcaatt ataaagaatt tctgacatta tcttgacatg aaaaccaatg gatagtggga |
| 44881 | cagaatgcaa aatcttcaag aatttttctt gttggttttt tttttttttt ttttttgagt |
| 44941 | caaggtcttt ctctgtggcc caggctggag tacactggtg agatcacagc tcagtgcaga |
| 45001 | ctcaagtgct cctcccacct cagccacagt agtagctggg actacagatg cgcacaacca |
| 45061 | cccctggcta atattttatt ttttgtagag atggggtctc actatatagt ccaggttggt |
| 45121 | ctcaaactcc ttgactcaag ggatccagga caggataaca ggtgtgagga gccaccacac |
| 45181 | ctggccatgt gcatgaactt ttaagacaaa cacaaggccc cacaaaagtt aaggttttcc |
| 45241 | cacctaattt ccaggggatc ttttggtgca aagctgagaa gcccttaaaa gtacacagac |
| 45301 | aactccaaag attcaagaga gttcattcgg ggtgagccag cccactgggc agactgacct |
| 45361 | tcaaaaaagg cccacccatg acacacacca gatggctctc caagaatctc ttcagtcctc |
| 45421 | agggtcccta aggcactgga cagagctagg aaagcaaacc catttgcttc tccctgcagg |
| 45481 | aaaccccttg aggtcaagac cccacaatca gacaaggatg gagtggctca ccttcagtca |
| 45541 | acaggccaga ctcaaggtgg tataatgtct taaccaaggg tgcagtcctc caggtctgac |
| 45601 | tcccaactca gttctccttt aataaccaca ctttgttaat tctccttaac aggagttcct |
| 45661 | gacaagtcag ttctccctca gaccttcagt ggcctcacct agaagatgag agggctggat |
| 45721 | cagatggaaa ttcggggagt aagggaatgt cggcacgcag cccacctccc ccaagggacc |
| 45781 | ctggagcctc catcccagtt cccaccatgc acccgcccca caaatcctgc ccaaggtgag |
| 45841 | ggctggtccc gggtcctccg ggtgccgcaa cagcgaggga aggagggagg ggaagcctcc |
| 45901 | aagggcgcga cgcaggctca aggatgcaac tcggccagga gtgaactggg gacccgaggg |
| 45961 | aggtatccgg gctgctcctc gagcccagcc cgggtccccg aaccccttac ctccagggtc |
| 46021 | cgtatctcct gctgggtgag gccgttggac acagcgcact tggtgcgcag cccgtgcagg |
| 46081 | ctgccgatgg agatgccgat gagcttctgt agctgcccgc actgctgcag cgtccggctg |
| 46141 | gccgcggccc ctgcgccacc ccatcacccc cgcccccgcc ctccttcttc tctcccatag |
| 46201 | cctccccgcg cagcgccgct ctatgcaggc cacagtggcc aaggcgggga gcccggggcg |
| 46261 | cgggcgccta ggcaaggaac cccggagacg ggagagctgg accaggagcg cccctcggcg |
| 46321 | gtgcccttac caggacgcca gtagagctgg cagccgagtc tgccgctccc gccctcagaa |
| 46381 | ccgcggcggc ggggacaaaa agccgcggcg gcgggggcaa aaaggcacgg tggctggggc |
| 46441 | aaaaagctgc aaaaagcacg gctgcggggg caaaaagcag caaaaagccg cggcggcggg |
| 46501 | gggaaaaagc cgcgggggca aaaagcagcg ggagcggggg caaaaaacca cagaaagccg |
| 46561 | aggcggcgag gggaaagagc cgcggcggca gggggcaaaa agctgcaaaa agcagcggcg |
| 46621 | gcaaaaagcc gcggcggcgg gggcaaaaaa accgcggcgg cgggaggcaa aaagccgcgg |
| 46681 | cggcggcggg ggcaaaaagc tgcaaaaagc cgcggcggcg gggacaaaaa gctagggcgg |
| 46741 | caaaaagccg cgctaacggg ggtaaaaagc cgcggcgaca aaaagccgct gagagggggc |
| 46801 | aaaaagcagc gggagcgggg gcaaaacaca aaaagccacg gcggcggggg caaaaagctg |
| 46861 | caaaaagccg cggcggcggg ggcaaagagc ctcggcggca aaaaccagcg gcggcggcgg |
| 46921 | gcgcgaaaag gcgcaaaaag ccgcggcggc gggggcgaaa agccgcaaaa agcagcggcg |
| 46981 | gcggaggcaa aaagccgcgg cggccggggc ggaaagccgc aaaaagccgc ggcggggcag |
| 47041 | gggcaaaata ggagaaatga ggtaggaggc cagcacaact tggcattgct ggagtgtgat |
| 47101 | gtgataggaa aagtgcagcc aaagacaaag aaagatgtaa gttggcttga ctcagtgcag |
| 47161 | ctaagaaccc agatgttatc ttgagggtat taactaataa gcagtttaaa tcagaatggc |
| 47221 | acattctgat ttgtttttta tattcacatt tggcaggcat agatactgtt tgaagagagt |
| 47281 | aaagtcagta gatagaggta acaaacttaa atatgtgcca agtctagaaa caagagacta |
| 47341 | gggggataag gacctttcaa aagaaaatgc aagatttgaa aactgattgg ctgggggatg |
| 47401 | aggaaaaggc acgtctttaa ggtcaatccc tgttttgctt taagttgtta gggggtggtt |
| 47461 | ttatcacata ttgtagaata tgtcatttca gttttgaaca tcttgagtta aattgtccta |
| 47521 | acatatttta tgaatttgat tttcttccct gggaagctag tatttcaaaa acttaaagag |
| 47581 | tatagatttc caacttgtat ccaatttata aaactatctc taggctgctg gtttcaggag |
| 47641 | gaggctcatg aatattctat ttgcagagaa tatatcagga gttaacaaca gcgtcagtat |
| 47701 | ttgtggacga ccagttaact aagccacctc ttagtgtatt tagataggaa atcttagctg |
| 47761 | aagatattca ataatgaacc aacagtgact aaaaaattca atatttaagt atatttcatt |
| 47821 | gcaattaatt tgaatttaag tagccatata cagctagtat ttactacatt gaaaaatgca |
| 47881 | aacaagagga aaaaattaat aaccatccct aataccacat gccaaaatcc tcatcaattt |
| 47941 | attctagcta aaggagttga tcagaagcac caatttaaag caccaatcac tgtcgttctc |
| 48001 | tcagaaccat ctcttctctg aacaaaacaa gtagaagagt taatagtgaa tctgcatttt |
| 48061 | ccttgcctat tttaaggttt tgatgttgac actaatttgt gaaatccctc ctgtggtgtg |
| 48121 | atatttcgtt ttccttgctt tttgttagga caagaatgct tcagctctta atttaaaatt |
| 48181 | atgtttctcc ctcccaggtg gagtgaactt agcatgcatt ctctgacata tccaagtttt |
| 48241 | tgttaatatg aatttggggg gaaaagcata cttaattagc taagacttct tattctaggc |
| 48301 | ttgaccctgt gttcgacatc tttttaattt gtagttgcat aggctgctct ctgacactgg |
| 48361 | ttagtgatct ggaagctata ttaacgttag gagaggtggt gtatgagcac tagaggtatc |
| 48421 | cttgcaaggg aagacttgtc ttatgtcaat acgtcttttt tttgcacaca agaaagtcaa |
| 48481 | tgtttgagtc ttctaaaatc ttcctatttc caagttgcag agtacaattg attcctaaac |
| 48541 | aacgatctaa tttttgactc agagacgtgg caaggtagtg aatcaccatt ataatttaac |
| 48601 | aatcttcaag ataaaattat ttctctgata tttagatttt gcccaattat taagatattt |
| 48661 | gggtgtttcg ttaagaatgg aagactctag tctcttgagc agagactata aaggcctcag |
| 48721 | atgatcattt ataattttat gctcttttct ttaacacctt caacacagtt ggaagcagcc |
| 48781 | aatattcccc agtgttgttg tgttttttaa accaaatgca tggttcagtg gtagaaaact |
| 48841 | gggctgatcc aagctgtttt cagtaaacac ttcatttcag gtgacctatt tcatattaaa |
| 48901 | taatctctag atcctgtctt caaaactaac tagatcagat aacctaccct agattttccc |
| 48961 | ctttagggtc tgttagctgc agtcactttt gtgaaaatga ttgcgatgaa aagatagagt |
| 49021 | tgtagatggg gaaaatgttt tgactaattt aagcatagtg gtatttaata tgagaattta |
| 49081 | agttacacac atttgaaaat tataatggag tctcttggct gagctttaaa aaaaatagcg |
| 49141 | tttaggctaa aaagggaact gctacctctc ctaaaatcag aaagatgtta cagtaattct |
| 49201 | ccattctcta gaattatcag gaagcacctt tgtgatgatt tacttttgct cttgggagtg |
| 49261 | tgagcctgtg tagtcgtgga accgtcaatt agagtgatgg ctttctgatc ccaaagtcat |
| 49321 | tcgttctgaa aacaatattt ttcataaatt tgaatgtgag aagttttgat cttgccattc |
| 49381 | ccaagtaact ctcttaataa gaggcatcag catgcttcag tgacagctgt caccttccag |
| 49441 | tgctgagagt catctttgag ttctccattt cactccctac actccaattt agctgcagtt |
| 49501 | ctcttggcca gtcctatgaa atacatccat ggcctaacga cttctcacca ctaataccac |
| 49561 | tcatacttac agcattctca cctaagtcac tacctttttt ctctggatta caatagcctc |
| 49621 | ccaatttatt tgctcacata acctatttat tctacacagt gcaccagata cacccctttg |
| 49681 | aaatgcaaac ggaatcatat tattctctgg tgaaattatc tcatatattc ctatcgcatt |
| 49741 | taaaattaat tcagaataat cccatgatta tcaaaaccct atgtgctctt ccacaacatg |
| 49801 | atttacttcc aagatatctc ttcttcaact tttttttcac tgtactgaat tggtgactaa |
| 49861 | aagtcatatt tttgtttttg cttaaaaagt cttgacttgt aaatttttca gtttctcctt |
| 49921 | tatccacagg taactctttc ctcataaggc gaattgcttg cttccttgag ttctgctctc |
| 49981 | aaagataccc ttcatgttct acctaatatt aataacttta atcattcatt attccattac |
| 50041 | tatgctctat agtgtataca atttctgttc tttgtcctgt tattaactaa attatttatt |
| 50101 | tgttccagta acgtattcca taaatattgt acacataaaa attatgttat ttttattgct |
| 50161 | gtatgctcag ctgcccaata acagtttgag gattaacata tttgttaaat gcacaaatac |
| 50221 | attctttcac aaatatttaa taattttata ttaaactccc tatatactta cagtatgaat |
| 50281 | tagataattc agaataaaca ttccattgga aaaaactaaa caatttgtta taaaacatcc |
| 50341 | ttaaaagcat cagaaggtta atacagacat gaagaattac aggaccaaat taagaacggt |
| 50401 | atggaagcct gtttgtgagg cttatgtttg ggttatctct ttacttagag tgactataaa |
| 50461 | tctcaaaaga gaactaaagg gagaaataac catatctact aacatggtaa gggtatttaa |
| 50521 | acatctctta gtaattgaga aaattgaaag aaaagaaaaa agaaagggag aaagagaaac |
| 50581 | agagcaaaag ggataatgaa ggagagaaag aagaagagaa aggaagagga agaaaagtaa |
| 50641 | aaaggaggag gagggggagg gaggaagaaa gaaaggtgaa aggaaagaaa ggtaaagttt |
| 50701 | ttaacagcat aatttatcct tgtagaatat gaatgttggt ctatttgatg atgtcccaca |
| 50761 | gattccttag tctctgctca ttttttatct gtttctcaga atcaatattt tccattttct |
| 50821 | tatcttcaag ctcatgactt tttctgtgtg tgcaaatata ctcttaaatc cctctggtga |
| 50881 | tttttaaatt tttatcattg tagttttcca ctccagaatt tctgttatct ctttgttgat |
| 50941 | attcctactt tttaatattt ttttctgatt ccttcatttc tttgtttatg ttttcctttt |
| 51001 | gacatttgac atttgagtat aattaagaga gttgttttaa agtctttgtc tagtaagttt |
| 51061 | gatgtctggg tttccttaga gatattttct gtcaatttat tttgtccctt tgaatgaccc |
| 51121 | atactttccc gttctttgta tgccttgtaa ctttttttga aaactggaca ttataataat |
| 51181 | tataattact atgtggttac tctgtaaatc agaccccccc ctacaaacat actaacgttc |
| 51241 | tgtggtttta aattttattt acttattata ttgttaagga ttttttttta gtgaaatttt |
| 51301 | ccaaagtgat taacaaaact gtttgcttta taaggtgtgg tcacccaagt ctttttgttt |
| 51361 | ccttaacaaa tgttaagata atgttttgac tgattttctt gtatgtcagg aactaagcaa |
| 51421 | acaggcaaat acaacaaaaa caaaaagaaa aacaagtaat cattatccag caaaataggt |
| 51481 | ctctaggcca tgcagactgg ctttgtgctg ggttctttaa agccgggaca aagtgtgtgt |
| 51541 | tcactcttgc actgagtgaa gttcaagttc actcttgcac agagcctgca ctgaggggag |
| 51601 | ggatcagcga aggtaaaagt gtagggtctt cttatgacat ttgtcagcat gtggcttaac |
| 51661 | gtatgcatac atgtgacttt ctagactctc ccatgtacgt gaatgatttt gaatgtctta |
| 51721 | gttttccata tactctactc caacttttct tcctgtgctg aaggtgatct actatatgtg |
| 51781 | taaactctaa tttttgccct aagcatctgt ggcttgttag gtctccttgt agagtttctt |
| 51841 | aataatgtcc attccttatc tgttctgtat cctagcaaca cacacaaaaa agcctttcat |
| 51901 | tagtccttta ggtatccccc agaccagtca gaacagacac ataataattt gagggtaaga |
| 51961 | tcttctcttg ttcctttgga cgatggacca ggtttcctca ctgggaacgt gggcttctga |
| 52021 | cacttcaaaa cagccaattt gctggggcaa aggcaagtta aaaacgtcat aaagttttca |
| 52081 | agttgtcttg ttcttgagtc tgctttcact tggttgttgt aatcttttga ccattttcca |
| 52141 | gagttttggc aaagtttatt cggacagttt ctcttagttg tgtgatgttt ctgtggggaa |
| 52201 | gtgaaagatt gcagttgtct ccactgccat tttgctgatg ctcctctttt gtcaattttt |
| 52261 | gcttcatgtt cttatgcttt gttattagtt catgtattag ttttctaggg ctgccataac |
| 52321 | caagtaacac aaactgggtg ccttgaacaa cacaaattta tagtcttata gtcctggaag |
| 52381 | ctaaaagtct gagactgagg tgtcagcagg gatggtccct tcaagggcta tgagagaaag |
| 52441 | tctgttctgt gccttgtttc tagcttctgg tggtttagtg gcagtctttg gcatttcttg |
| 52501 | gctaatctct gccctcataa tcacatggta ctctccctgt atgtatgtct ccctctactc |
| 52561 | aattttcttc tttttataag gacatcagtc atattgaatt caggctcatc tgattttatc |
| 52621 | ttaacttaat cacctgcaaa gaacctattt cctaatgagg tcatattcag tggttaggat |
| 52681 | ttcagcgtct atatagatga aacaatttag ctcatatttg tgcatacatg attgtaatag |
| 52741 | ctatgtcttc caaaagtgct gaccccctta ttactacaat ataaattttt aaaatcctat |
| 52801 | tcacattttt aatagtctat atcgtgtgtt atgagtataa tgagttcagt gttcttatga |
| 52861 | ttgctctttg catgatattt tttgtcatct ttttactttc aatccattag tatccttgcg |
| 52921 | tctcagcgta tattgggatc acttgtttta atccagtctg agaatctctg cctcttgaat |
| 52981 | ggattttaat ctgctcacat ttaatattat aattggtata attctattta tgtctgccat |
| 53041 | tttaccgttt gttttgtgta tttctcaaat atttttcttt attgctttat tttgcaatga |
| 53101 | atgaatattt tctaaaatat ggaactttag attactaatg aattatttta gtatatattt |
| 53161 | ttgggaattt ttgttgttgt tgtaagttta ccatataggt atatggaaaa ttaattattc |
| 53221 | aaatcatctt ccaatttata ctagtaaact tttagtaata catagaaaca tcattcttat |
| 53281 | acaaatctct tatatttcct ccattttaaa gtattatcac tttacacatt atatctatta |
| 53341 | aagttacaaa gccaacaata cattttagta attactactt taccatctag agtgattacc |
| 53401 | ttatcacaat acatttttct tccaactacc tcctctttga tgttactgga aaatatgtta |
| 53461 | tagatatatt acatttctac atgtcaaata ctcagcaata cattatgagc atattattat |
| 53521 | tattattatt atcattaaga cggagtctcc ctctgtcacc caggctggag tgcagtggca |
| 53581 | caatctctgc tcactgcaag ctccatctcc tggcttcatg acattcttct gctttagcct |
| 53641 | ccggagtagc tgggactaca ggcacctgcc atcacgtcca gctaattttt tgtattttta |
| 53701 | gtagagacag ggtttcactg tgtcagccag gatggtctct atctcctggc ctcgtaatac |
| 53761 | gcccacgtcg gcctcccaaa gtgctgagat tacaggtgtg agccatcctg cctggccatt |
| 53821 | atacgcatat tattttataa acaatttatg ataaagagaa aacatggatt tctactgtct |
| 53881 | tttataatgt taatattacc tataccagtg cttttttaaa aatgtggatt caaacgactg |
| 53941 | gcttgtgtaa cttgctttta gccttaggaa tttattttag tgtttttttt tttttttttt |
| 54001 | tttttttttt ttttttttgt atggtaggtc tgccagcaac accttcagtt aatatttctg |
| 54061 | tttatctggg taagactttg tgttatcttc atttttgaaa aataattgct ggataaggaa |
| 54121 | ttggtggctg acagtttttt ttcctttgca tcttttgaat atattattct actgcctctt |
| 54181 | gccttccatt gtttctgtta agtcagctgt taatcttaca aaacctaggt gttcaaaaaa |
| 54241 | taaacatgtg catgaatatt tatagcagta atattcatac cgtcaaaaag tggaaacaat |
| 54301 | ccatatgctt gtttactcat aaatagacac ccaattttca gctataacaa agaatgaagt |
| 54361 | acttatacat ggtataatat gggcgaaatt tgaaagcatt atgttaagtg cacaagagga |
| 54421 | caaatattac ttgattttat tcacatgaaa caccaggaat tggcaaatta attgggatat |
| 54481 | aaatcagatt agtggtcatt agggctcagg gaagcagaat agggtgtaac aactttatgc |
| 54541 | ataatgggtt tttagaaggg acatgacgaa attttcctgg aacattgtga atatactaaa |
| 54601 | agcaagtgca ttgtgcattg tatgctttaa aatggttgtt attaatttta tattatgtga |
| 54661 | ttttttacct taaaaaacaa aaaagagaaa atagccttac tctatataca ataaattcaa |
| 54721 | gatgtgttac aaatttatat gtgaaatcca aaatagtata atatttaagg aatagctgag |
| 54781 | tagaataaca ctaaaattta acataatgaa atatttcctt aaaaaagaaa aaagcacagt |
| 54841 | aattaaaaag ggaaatatag ttaatatttt ttctctccat taagcatgcc attaactgag |
| 54901 | taaaatatca agctgcaata tgtaaactgc attttctaaa accataaaga aaataagaaa |
| 54961 | tgaaaaggga ttggggaaaa aaatccaaag gtacagtcaa ctacacaaaa aaaccttagt |
| 55021 | ctcattaatc agtatgaaaa tgcaaattgt aactgaaata agataaaact acaattcaaa |
| 55081 | gagaaagcct aaaatttcaa ccccccaaaa attctgggtt ttggagagct gggatggaat |
| 55141 | agggctgcta accttacaac aatgaaagaa ccaaactaac ttcaaagtca tgactttatt |
| 55201 | tttatagtaa ccaggttgcg aagaactgag taaaaatgtg agggaaaaca agcaactgca |
| 55261 | aggagaaaga ggacagatgc acttacatag gacagatgca aatagacacc actatgacaa |
| 55321 | gtaaagctgg aataatcaat aaattcctaa agacaaagtg gggctggtca gattgggaga |
| 55381 | ccgctgacag ctgcagaagt tgggaaagat ccatcatctt gaaaactttt tccccacaaa |
| 55441 | cccactgtga tctctcaagc aattggtaag gaatccaaga cagtctgtat atgatacaga |
| 55501 | tcagggagag cagaacactt gggaggtgac caggtcttgg gggccgagcc cttatgaatg |
| 55561 | ggattagtgc ctttataaaa gaagctcaat ggagttcttg tgtgccttcc actatgtgag |
| 55621 | gacatagaaa gaaggcacca tctatgaacc atgaaatggg ctctcatcaa cactgaattt |
| 55681 | gtgagcatct tgacctgaga acttacagcc tcaagaagtg cgaaaaaaga aatatctgtt |
| 55741 | gctttttagt cacctggttt atgttatttt gttataagag tccaaataga ccaagatatt |
| 55801 | ccattccact taatatgtag gggaagacaa caaaaactgc cacacttaga atactcctga |
| 55861 | tgctgggagt atgaaaacag gaaaaacaaa acaaaactgc tcttgaaggt gaaggaggaa |
| 55921 | tatcactgag ctcaccaaca cagcaggaaa agaacagtga gaaggctaca ttcatgagac |
| 55981 | cctgagaaaa agtacctgca taagactgag atgaaattac ctaccctagt tataattgaa |
| 56041 | atcccaaaaa gaaaagagga aaaaataatg gagcaaaaga aatatttttc aaaataactg |
| 56101 | ccaaaaatat tctaaaagaa gtgacagaaa atcaaacttc agatatagga aactcagaga |
| 56161 | atgtcaaata gaacaaaaat aagaattaca tcttgaaaaa tctttaaaaa atcaactcta |
| 56221 | aattttatat cttgctccaa atatatagag atataaatag gttatcatcg agatatggag |
| 56281 | aaagccatgt catggaaaca ataaaataaa gctgtggaag ggctacattg atattagaca |
| 56341 | caacagagtt cagaacaaga aatagtatca gagttgagaa gtaataagta atataataat |
| 56401 | caattcttaa gaagatgtga acatcctact aattagggta tgcagctaac aacagaacct |
| 56461 | ccaaatacat gaggtaaaac aggaaagaaa tcaaaggtga actagaaaaa tccaaaatta |
| 56521 | tatttgcaga cttcaacact tttgtcttag taatggacag actaggcaaa aactcagtaa |
| 56581 | tcatatggaa gataagaaca acaatatcac caacaagaca tccaatcttc aatggcagat |
| 56641 | actctttcct ttcaagtgaa aaaaaaaaaa aaaacaacaa cagtatggca tattctctaa |
| 56701 | caaacccaga atttctaata tttgcggtct tccttccttc tttccatctt cctttctctt |
| 56761 | ctcttccctt cccttgcctt cttccttcct ttcttctttt cctctttctt ttctctctgg |
| 56821 | ttttctttct tttctttctt ttttctcctt ccttccttct ctccttcttt ctttcctcat |
| 56881 | tctttcttcc ctccatcctc ccttcctttc tccctccctt cttttcttcc ttttctctta |
| 56941 | ttctttcttt ctcactttct tgctttcttt ccttttttct cccttccttc atcccacctt |
| 57001 | ttcttccttc atccctccct ttctttcctc ctttttcctt ccttacttcc ttctttactt |
| 57061 | ccttccttct cctctttatt ttctttgttt cttgcctttt tctcttttaa cattctctct |
| 57121 | tcctcctttc cttcctccct tcctccttcc tttctttatt ctttctttct tgtttctttc |
| 57181 | tttctttctt tctttctttc tttctttctt tctttctttc tttctttctt tctttctctg |
| 57241 | tctctctttc cttcttgtgt tcttgctttc ttttttctcc cttcctgcct ttctcccttc |
| 57301 | ctccctccct cccttccttc tctgatttcc tccttctttt ctttcttctt tctttctttc |
| 57361 | cttcctttgt tctttccttt cttctttttt ctttcttctt ttcttttctt tctttctctt |
| 57421 | tactacaatt catattattt aaaaaattta agagagggag gcagaaaaat aaagaacact |
| 57481 | ttaatctgca ggtaaataga ttatgtctgc tgtatacaaa agaatggcct cccaaaaatg |
| 57541 | ttcatgtcct aattcccaaa gtctaacata caaatatgtt aggttgcatg gcagtgggaa |
| 57601 | attagatttc aagtgaaatt aaggttgcaa taaaatgatg gagagattat cttaaatggg |
| 57661 | tgggatcaat gaaatcacaa gcttccttat aagtgaaaga agaaggcaga agaaaggcaa |
| 57721 | ccatggaggt ggtggcatga gaaattactc aacatcactg acttttaaga tacaagaatg |
| 57781 | aggacccagc gcggtggctc acgcctaatc ccagcacttt gggaggccgg cgtgcgtgga |
| 57841 | tcacgaggtc aggagatcga gaccatgctg gctaacatgg tgaaacccca tccctactaa |
| 57901 | aagtccaaaa aattaactgg gcatggtggc acgtgcctgt agtccaagct actcaggaag |
| 57961 | ctgaggcagg agaatcgctt gaacccagga ggcagaggtt gcagtgagct gagatcgtgc |
| 58021 | cactgcactc cagcctgggc gacagaaggt gactccatct caaaaaaaaa aaaaagatat |
| 58081 | aagaatgagg tcatgttcca aggaataaag gtggcttctg catgctgaaa aaaatcaagt |
| 58141 | acatagattc tgccacagag ccctcagaaa gactgcagcc ctgcccaaaa cttgatgtta |
| 58201 | gccctgtgag tctcatttaa gccttctgag ctccagtact gtaagattac cagtcacttt |
| 58261 | actgtaagat atgaagtttg tggtaattgc ttacaacagc aagaggaagt ttatatagta |
| 58321 | attgtatcac gaaaatgaga accataattt acaactgctt ttaatactgc acttggatgt |
| 58381 | ttgaaatcac gtacatggaa atgatcagta tgtgtatgag ggaagatagc aaattgatgc |
| 58441 | caaaataacg caaatgcaaa tcttacactc atttctatgt aggtttcatt taatctttga |
| 58501 | aattaaaatg aaattaaagg attatgatat tttgatgaaa ttagactaaa atgaacaata |
| 58561 | acaaaataag aacttacata tattccttat atggtcaata atgaagtgat agtggaaaaa |
| 58621 | aacaaggtca aatgaaggtg atgatttagg aagttggaaa gatagcttaa aatacaaaat |
| 58681 | ggtgtataac tagtgaacac ttagacacac tgattgatga acttcagctt ttggcttgat |
| 58741 | gagagcataa aatgagagca gctgaggttt gcaaatttgt aatctgcttg tggaaaaaca |
| 58801 | ggggaaaaca catctcagcc taataagatt tatctactaa agagtcaaga attgatccat |
| 58861 | ttgtccttgt aattcaaaag ctaattcaaa tactgatttg atgtattgtg tgaacaatca |
| 58921 | ttgctgatta tcatcgcata cctggcattc tcttttatct gatatctaaa atatttggta |
| 58981 | attcctggac tttctctttt caaacccagt atggattaat ttcaatctta gaacagttgt |
| 59041 | ctttgagaaa ttcttccctc tactgcatct gtgaatgggc atagcatggt tacctacata |
| 59101 | ctgtcgcccc agagaacatt tgttgaatta aagccaaagt ttaaagcaac agctttaact |
| 59161 | cactggtttt actaatgttt tcctccccat tagccacaac aatattgata ccctcacacc |
| 59221 | ctttaacata aagcttggtg ttgtctattt ttcacgtgct gtcatctata tgatctcagt |
| 59281 | attttaaaaa tcagcttcca gcccatatgg tggctcatgc ttgtaatacc agcagttgaa |
| 59341 | gaggctgaaa tgagaggatt ctttgagccc aggagttcaa gagcaacctg ggcaacataa |
| 59401 | caagacccag tcttcatcaa aagttaaaaa aaaaaaaggt ggccatggtg aggtgcacct |
| 59461 | gttgtcgtag ctatttggga ggccaaggtg gaaggattgc ttgagcttgg gaggttgagg |
| 59521 | ctgcagtgag cagtgattgc accactgcac tgcagcctgg gcaacaaagc aagaccctat |
| 59581 | ctcaaaaaat atatataata aaaataaaaa tcagctctca ttgatttcta cataaatgtg |
| 59641 | aacaggtgat gtccatatag acataaataa taatatatct gacaatggtc catatgatct |
| 59701 | tcaaaatgta aaatgcctat ctgtgtaatt gactggttag tctcattaat gaatatagat |
| 59761 | tcaattctac tttcttgttc tagataaatt atataatcta gcttttcctt tcactcattt |
| 59821 | actgataaca acaggaagaa tgacaagata tctattttgg aaaattactc tggtaggagt |
| 59881 | aaagatgaaa caatgataga attgcatgga aaactagaaa aaagtatggt cttctgatat |
| 59941 | tatatcacat catataataa agccctcata caactcagat attttatcta aaaatgttat |
| 60001 | tttcatctta ggaatgatca cagcatgaga ctagaattgt attacaatga ctctcacaag |
| 60061 | cacatgtgct aaaaaggaga ggaaaacatc attactgata ttttaaacat atgttttact |
| 60121 | ttccatcaac gtgaacctca acttgatatg atgcagattg aaggaaatca cccataattc |
| 60181 | catatgaaga aggcctgtga tattttatgg gaaaataaat agagaaaatg ctaacagaaa |
| 60241 | ccctattaag cattaagctt tatggagcaa agacaaatcc agtggtgaaa gatacacact |
| 60301 | cgagttctgt ttgttgtctt ggaacaatac ggtttagagg tgactggtgg gtgaggagaa |
| 60361 | catatgcgag ttcaccaaac agaaaagctg aatgaggcaa tgcctctttc tgaccatatc |
| 60421 | tcttactcag ataactatat aatttattgt ccagtaaagg gtatatttaa aaatcatatt |
| 60481 | aaaagtcatg caatgaagtt gtccagggaa atcaagactt aagagtctca ctctgacaat |
| 60541 | aatgaatggg gggggttccc tcaagaaaga ctaggacatg actccacact ggcaggtagt |
| 60601 | agtaccagaa aagaacccat ggaaaatctt taccttatgc ttgaggtagg gaccaggcta |
| 60661 | aagtgaaagc cagacataaa attcaatcta aaatatatcc aaaattgaag aaaatatgtg |
| 60721 | ttgtacaggc atagaatgtc tttcctggat cattgaaata gtaagataaa ttcaattttt |
| 60781 | tacattgttt tattttcctc cagttagggc ttgaggtttg tctctggaga gagactgtca |
| 60841 | attggagccc tgcctttctg gggttctggg cagggggttg tggatgctta acatgtgcct |
| 60901 | ttcacaggac acttccttac cccagcagtg gccaggtgtg catcccacga gcaggcctcc |
| 60961 | ctctcacaga aaatctgttg agactaggag atgcctggtg gctgttgcgt gacctgtgtc |
| 61021 | ctgtgtattt ctgacaagag ccactctcag agaccctggc caggaggaga gttaggttcc |
| 61081 | agtgtaggtc agctcagaca catggaggcc acaggaccaa acatgggaaa tcacagaagt |
| 61141 | agttttatta ctcacagatc cagagagaag agggtagctg agaagagggt ttagctgtgt |
| 61201 | ccccagccaa atctcatctt gaattcccac atgttgtggg agggaacagg tgggaggaaa |
| 61261 | ttgaatcatg gggacaggtc tttcccatgc tgttcttctg atagtgaata agtctcacaa |
| 61321 | gatctgatgg ttttataaag gggagtttcc ctgcacaagc tctcttgtct tgtctgctgc |
| 61381 | cacgtgagac ctgcctttca ccttccacca tgattgtgag gcctacccag ccatgtggaa |
| 61441 | ctgtgcacct attaaacctc tttcttctgg aaatcaccca gtcttgggca tgtatttacc |
| 61501 | ggcagtgtga aaatggacta atacagtagc acacctcata gggctgaaca aaatgaggaa |
| 61561 | gataagtggg gagcaagaga gaaaaaagtg gtctgtggga ctccagcctt tattgagccc |
| 61621 | agaacattac ccaaataagt tttccacggg gctctggtca gtggggtgag agccagtagg |
| 61681 | cacatttctt ggctgcagct gcaactgagc aggtcactct ggcgtgtggg ggctgtccat |
| 61741 | gtgggctgtg aggtctgtgg ggtgagtcag gtaggttgta tccaacagtt ccacaacggc |
| 61801 | tagtcaccag gaggaggcag ctgtgtaggg tcaatatctg ggccagccac actgaggaac |
| 61861 | tgtgagggtt agaactggaa atcatcaagg gaatctgaac ccagctacca tatgagagag |
| 61921 | ttcaacttac gttcaatgtg aatgccatgg caatattaaa aggtaagaat tcgctccata |
| 61981 | cgtgtttgag gtaaatagga gaaacctaga atttatgtaa acagtgagaa gattggatgc |
| 62041 | gttttccgtc acatatttta atactagcag catattatat atgtcaatcc atcaggcatt |
| 62101 | cagaagtact tgcttatgaa aactttttgc accatcagac aaaagacaag ggtagaagac |
| 62161 | atttttaacc ctacaaacac tagtaaatta aaaacagaag gacctttatg tcctaatatg |
| 62221 | tctatgttgt gaaaggctgc cctgtgaaat acaggatttc ttaaacatat tttaaaaatc |
| 62281 | atagctgtca atatttttta gaaatccatt taaattttct cttgcttttt tacaattcct |
| 62341 | atttatttat ttagtggttc tgttgatttt gatgtatagc ctaaacttta tatttcttta |
| 62401 | aagtatgttt tatacaactt tatgtaaaat gtttgagtat cttcacgttc tcaccctgtc |
| 62461 | cttttgtttt gctcttatat ggtggccttg agtcttttct ctggcttttc aaacctagta |
| 62521 | agactaagac accaaagtaa ctttgcccgt ggtttcgtaa tgccttctaa agcacatcct |
| 62581 | aagctctcgt gcatacaggg gtctcctttg agctctatgc ttttgagatc ccatatacct |
| 62641 | aaattccagt actccaaatc agtactgctc agttttagtt actaagttta aaaatgtatt |
| 62701 | ttaataacaa gttagtttag ttcactcttg cttctttctt tactgctggt atacatgtat |
| 62761 | attcctttaa ataaatcttg gaatttattt aaaaatttta aattatacta atgaaactgt |
| 62821 | atattgttgt ggattcatag gtgaatttgg aaagaatttg tctttgtgat actaaatcct |
| 62881 | ttttatccaa gaatcatatg tgcctttata tttattccag tctatattta tatcactgag |
| 62941 | taaatatata gaaatgtaga tacatacagc tgtagctata gatagataca aatatagata |
| 63001 | taacatgtta aatctatatc tatcccatat aacatatatg catgttatat gtgtgtgtat |
| 63061 | atatatatat gtttgtgtta ttaaagagct cccttaaaat ttttctttta tttcctatat |
| 63121 | aattttaggt tgagcttgaa ttttctttgt acaaacaagc aaatatttat actagtttta |
| 63181 | atactgaggt ttagacattc tatcttatat tagcattgaa tattttcaca attataaata |
| 63241 | ttatctaata ttaataatgt aacttaaaaa tatttaaaat tttacctttg aattatttta |
| 63301 | ttgttgaatt taaattcctt taagtatgac agtaaatttc tattttatgc tttctctatg |
| 63361 | catatgaaat taatctattc acttctctac ctttatgtag taacatatga aaatcaggcc |
| 63421 | tctgttcttc taatggacat acacatgttt gcatatagaa tatcagactc tttatagtat |
| 63481 | ttaaaatctt taaagacgtg aatatgacct tttaacaaat atattttagc gtgtactgag |
| 63541 | aatcccctat ttatttttta tttgggctaa tcaatatgat tattaatatt attggattac |
| 63601 | caaatttgga atcacacttt catccccaag gtggatattt gttttatttt tttgccaatt |
| 63661 | tcttgtctta ctgtttcaaa tactgttgga tattattttt attttatttg gcattttagt |
| 63721 | atcaatattt gtaagtgatg tactctacat attttttctt caatatctgg tgggttttat |
| 63781 | aattactgct atattggatt tgtagtagac attgagaaaa attattcctg catgttttat |
| 63841 | agctgtatga aggaaactaa tatattttac cccaaaatat atttccttga tatatttcaa |
| 63901 | aatggctatt gagaagggct ggaaatgcaa acatagctgc aaagctgtct tggggagatt |
| 63961 | tgcatcagta gagaatctgc cttgatgcag ccaggctttc tctgagatct gttcccttgt |
| 64021 | ctggatctag gaaagattaa ctgagggtct gaggtctgca aaggtctgaa agaaacattt |
| 64081 | tctgtctatt atctctgagg actgctccca gtgaggtttc acctacgtaa taagtccact |
| 64141 | gttgctagcc agggtccttt tctcacataa cttttttttt tttcctctgt gatccaagac |
| 64201 | cccattcttt ctgtaaactt catgtggtag ttaaacttct gcacccatcg tgtgtctggg |
| 64261 | tcttcattct aagggctcca gtgtacacac attgcagaaa cctgtatgcc ttttctacta |
| 64321 | tttatctgcc tcctgttagt gattttcagg gaaacttcag aaggcaaaag ggacattctc |
| 64381 | ctttagccca tactcagaca aaatccccca acatttaact gattcctaat agcttaaaat |
| 64441 | cactttgaaa actccacata tttataactt ttcttccctc tatgatttct gttcagcttg |
| 64501 | ggttttgttt tttattccat ttacttcatc ctcgaaagac ctattttatg tctatttatt |
| 64561 | ctcatttatt gacattgaga aaagaaaata actttcatgt gagaaacgca agtcctttta |
| 64621 | aataatcagg cccagagaga tattcagatg agacagcagt tctgttctgc tcctctttga |
| 64681 | gctgtgtgtt catctaggct gccgctgttg ccacagtagc tataaattaa ccaataacgc |
| 64741 | cacaccagac actataatcc acacccaata atagtgtaac agtgtatagc cagtcactaa |
| 64801 | taaatgttat ttccataagc caatgagaat ttgtgacaaa cctctttgca tcatcccact |
| 64861 | tcttgtccct tttttgcctt taagaaactg cttgttgcaa agctccaaag ggagttcata |
| 64921 | tccaaggata cttgggtctg ttccttccag gcagctgtcc tcattgtggc tcaagtaaac |
| 64981 | tctttgaatt acgttttgtg cttcagcccc ttccacttag attaacaaca tggatttgtg |
| 65041 | tcaccatgta cagcaattaa aatgtttaca cttttcccct cgagggcact gatgtgtttt |
| 65101 | cctgagcact tggaatagct acttagtgtt tactttctag aatatggttt ctcaaccttg |
| 65161 | gtgctactta cctttaggac cagaggattc tttgttgtgg gaggctgccc tagcaatgct |
| 65221 | aggtgtttcg tttgacctct aaatttcaca cctccaccag tcttgacatc cccacaataa |
| 65281 | ccctagacat tgacaaatgt ctcctgggga aaactccacc agttgacagc caaagttctt |
| 65341 | gaaatattgg aatcgtcaat tgagttttta tgttatccaa aacaaatatt tttctttgtt |
| 65401 | tttaaacatc tacttccatc tacttatcta cttattttta cttttattta taacttaatt |
| 65461 | ccatcaagga gagagagtgc attttgtgtt atgctaaatt tttgaagaat gtattgattt |
| 65521 | tttatggcct gatatatgga tgatatgtag atattacatg tttgtattat caaatttcag |
| 65581 | ggtgataata aaataaatac ttataatatt tatattgtca ctgtatatta gttatttctt |
| 65641 | tcttcactac aggagttttt caacctatag gctatttttc aattctaggt tatccagtag |
| 65701 | attttgaaat gttatgatta aatatctact tctcaagcat tcatctttgc aaaggaatca |
| 65761 | atcccaagct cttataatgc acatcatata aagggcagat tagtcaatat atggttcaga |
| 65821 | aataattatg taatatttat aagaaaatta aaaatttaga tccttaactc agataacaat |
| 65881 | aatccaaatt aaaatttgat ttaattacat aatttaaaat gacaccagaa tactagtaaa |
| 65941 | aatgtagata agtttatata atccttttta gctgtagaac tttattagca taaattcaaa |
| 66001 | tacaggaacc aaagtaagat tgagacctat agtcaaaggt taaaatgtac acattatagg |
| 66061 | ggcatgatta aactaattta aagcatagta acatggagaa atattgcaaa acatacattt |
| 66121 | tactgaatta attgttaata tctaatcatt acatgagaac aaaggtaaag agtagctaca |
| 66181 | cacacacaca cacacacaca caagtgcaat attttcagta aacgtgatgt tcagctacac |
| 66241 | tacaaatcac acctatgttt tctccacaga aaagtaaaga ttaaaaatca caataatatt |
| 66301 | tattgtacat atggaggtaa agatactcaa aatattaccc taaaatactt ttttttttga |
| 66361 | gatggagtct tgcttttatt gcccaggctg gagtgcaatg gcacaatctt ggctcactgc |
| 66421 | aacctcagcc tcccagggtc aagttattct cctagctcag cctcccaagt agctgagatt |
| 66481 | acaagcatgc accaccacac tcggctaatt ttttatattt agtagagacg gggtttcacc |
| 66541 | atgttggtca ggctggtctc gaactcctga cttcaggtga tctacccact tcagcctccc |
| 66601 | aaagtgctgg gattacaggc atgcgcccgg ccaacttttt gacatatttc aagatggcta |
| 66661 | ctcggaagac tggaaatagc ttcttctaca agaatagctg aaaagctgtg tttgttgggg |
| 66721 | agatttgcat ttgtagagaa aatctgcatt gatatagaca ggctttccct gagatactcc |
| 66781 | cttgtctggg tttaggaaag attaactgag tctggcacgt ttacatttct aaaaaccatt |
| 66841 | tcctatcttt acttcccaag aggagggctg ctccctgtga ggtttcatcc atgtaacaag |
| 66901 | accacctctg ctgccaggct cctctttctt ccttgtcgtc acctgccttc tgcaaagcct |
| 66961 | gatttagcaa agtacagctc tgtgttttct gtaacctcaa gacagcatag gcgtgttgac |
| 67021 | taccttgcct ttcctggagt ttttatacat atacagtata tatttgtata tcaatttata |
| 67081 | atatacaaat atttgtatat acatatttat gtatattatg taaactccaa gtgcatactt |
| 67141 | gtgcacgtaa ttatatctgt aaaccttttt tcctgttaat ttgtacatta tcagtttgtt |
| 67201 | tgatagactc aaataattaa agcttcaagg gaaaaattta aactttccta tagagaaaag |
| 67261 | acatatatgt gacaaataat atttagagtg taagacgctt tttaaaggta tatttgcaat |
| 67321 | ttgtgtcaaa acatttaaat atacatttgt tactttataa aatttcaaat aatttaagct |
| 67381 | aaatacatag tatatgcaga aaatttagca atatttgtat gtagcacctt actgtgcatt |
| 67441 | actgtaacca gctgtctaat ataaagaact aattaaggta gtacctactt ttcaaatatc |
| 67501 | gcattttttc acagacctat taaataagac aaataacatt taaactttat ttttaaattt |
| 67561 | gcagaatagt atttttcagc agatggttta ttttagcaaa ttccatcttc acattgtgct |
| 67621 | atgcttttat gagttccagc tgttaacgga taatatttta ctgctgaatc tatcatgtgt |
| 67681 | gatataattg ctcattatgt gccttaaaac acaagcgata tggttatttt caacttggag |
| 67741 | caaattaaaa tcttatcagc aatttaaaaa gtctagagtc gtcttcttct ggttaatttt |
| 67801 | tttaaacttg tatttttctc tttatgtttt tagtgagttg tcttatcaag gagaagaact |
| 67861 | caagctgatt attctttttt tttctcttcc atccacctcg ctggtgtgtg aataatttca |
| 67921 | tttctcagaa aatgttcttt catatccatc ttacaagatg agagaccttt taacatcttc |
| 67981 | cattcggatg tgataccagt aatggaaaat attccagctt catgaatatg gtgatacaaa |
| 68041 | tagttatccg tctaacctct gtcagtgcca aatgtttact ttactcagtg aattactcag |
| 68101 | ctgactggta atttcttctg aaatcactaa tgagaggatc agaggtctgg ctgttgtctg |
| 68161 | tacctcgtat gactcccagt gcagacaatt gtttctatgg agcacagaca gttgaatgga |
| 68221 | ttgacttcct gcctagaata gtttctgctg tgcttcttat ccttcttatg gagatttcag |
| 68281 | attatctgaa ttgcttttct atcttaagaa aaaactcaac aattctccca cctgagagga |
| 68341 | aagtaaactt tagtaagtta gcagaagcaa tccgtaaagt ttttacattg tttgttgtaa |
| 68401 | aatgcagcgt tggtgtctcc atcactaacc ttttctatcc ctcattgctg tttctttgac |
| 68461 | tgcaatagga tacctctagg caaatctgta ttcccgagac agagtgccct ttccgttagc |
| 68521 | tataagtaca ctcaatggta ggctgaaatg atagttttta tctatggtga aatggaatca |
| 68581 | tatcagtgat ttttttaaag gaattttaac tcttgctatg gtttgaatgc ttgccccttc |
| 68641 | caaatctcct gttaaaattt gatccccaat gttgcaggtg gggcttactg ggaggtgttt |
| 68701 | ggtcatgggg atggaacttc atgaatggat aataccctcc cttaggaatc tcaagctatc |
| 68761 | ctccctcctc ggtgccctca ggaatgaatg taccattctt tattcaacta taattccccc |
| 68821 | acccatccct tttgagatat taattaaatg tatgttacac tgctgcatat tgtctcacgt |
| 68881 | atcagtgagt ttctggcttt cttattttag tttacccttt gtcctttagt ttgtaaagct |
| 68941 | tctaattttt ctatatattt tctgatgtta gggtaaaata catgacttat tctatctcat |
| 69001 | ggaattttta tttaaaatat ttatttttca tctatacgtg tcacattttt cattttataa |
| 69061 | cttccatttt tctcctatgt tcaattttca tttaagtact ttgacatata tatgtattta |
| 69121 | tctatatgta tttataaaat atatttactt taaggacctt gaaaattcct tcttctgtca |
| 69181 | tttataaatg gcttattttt atcctgttaa tatatatctt aataatatat atcttctggc |
| 69241 | ttctttgcat gtcagagttt tttttggggg ggtattttga tgttatgcta ttgaatatct |
| 69301 | agattttatt ggctaccttt gaacaatgtt gtggcaggca gttcagtaac ttcaggatga |
| 69361 | gtatttttct gttgttgttt taaatcttct ctttaaactt tgttgagtta gtctagagcc |
| 69421 | atctgtaatt tggagctaaa tgagcactgt cactagggca taacctccag tggtctttac |
| 69481 | tgaatatcct ggaggtacag aggggattcc cttctctggc tggtcagagc taacatgtct |
| 69541 | tcctgtcatg tgacgccagg gaagtgttct tccaactccc tggtagagtc ctttgctgag |
| 69601 | ctccttagaa tttcatccta tgtacatttg gcttagggac ttgggagaaa ccttaggctg |
| 69661 | attattggtt cctttttctg taaacgttct cttctactac acattccagc tgcttaacat |
| 69721 | tttggttttt atctggttcc tcagtgcaat gacaatgtct gctctctctg ggattcctct |
| 69781 | ctactgctgt cacggagaac ctgggaataa agcaggactc attctggctc cttcttttct |
| 69841 | cttgcggagc acagtcctgt gctgcctgat gttcagtatt tcaaaaaaag tttcatatat |
| 69901 | tttgtccagt ttactattct ttaactctaa aagagtaact ccagtcccag ttacagcatc |
| 69961 | atgttctgta actctactcc ttgttgcttc attctgccat tgtctggtat gattgcccct |
| 70021 | ttcccttctg taatcaggcc aagagcataa cataatacta gttataactg cacagcttgc |
| 70081 | ctccgttgtg taaaaaaatc actgagactt aactgtgtcc aactttttaa atgtgaatat |
| 70141 | aagtacaact aaagctatat tttgtttaat atttgcattg catgcttttc cattatttag |
| 70201 | tttcaaaata tgtgaaatat aaatataaat tataaaaact ttaagagagt acatttaaaa |
| 70261 | aatctggtct gattatgttt taactggttt attacaacat gcattcttga attcagggtc |
| 70321 | taatataatt ggtacatttg tctatttgca aaaaaaactt gacaatattt taaaattaat |
| 70381 | ttatccaact cacaacttat atgcttctgc tgttgtatgg aagatgcatt ttaaacttta |
| 70441 | tgagctagca ttctgttata cagtcgatgt ccaattaaat ttctctctat gtttatttct |
| 70501 | ttcattaaaa aaagcgttct tctaactgca aactttcatc agggatcata gctcttctac |
| 70561 | ctgaagaata atctttagta tttcttatcc tgtgggtctg cttgggataa attctttatt |
| 70621 | gtatctttgc ttttgatgga tatgtccacc aagtagacag ttctaggtcg gcacttattt |
| 70681 | tatttcagga cttgaaaggt atcaatacct cacttgttgg ctttcattgt tcatttgaga |
| 70741 | aggttgttat cagtcaactc tttctcttta tagttagccc aattttttta tcaagtgctt |
| 70801 | ttgacatttt tcttttactt ttcagaaatt gtcccatcat gtttctaggt gtgtcctctc |
| 70861 | tgtgtgtttt catttggttt gcaaagcctc ctgaacctgt ggattaatat tattggtcaa |
| 70921 | ttttgataaa acctctaaca ttgccactta aaatgctgtt cagaccagct gttttgtcct |
| 70981 | tcttagattt caacgtgtta gattattact gtatgcttta tattttttaa ataacctttc |
| 71041 | tctacttttt ttttagttgg ttaatctgta ttagtgtatc ttttgctttt ttattttatt |
| 71101 | ttattttatt attatacttt aagttttagg atacatgtgc acaatgtgca ggattgcaac |
| 71161 | ataagtattc atgtgccata ttggtgtgct gcacccatta actcgtcatt tagcattagg |
| 71221 | aatatctcct aatgctatcc ctcccccctc cccccaccac caacagtccc cgaagtgtga |
| 71281 | tgttcccctt ccggtgtccg tgtgttctca ttgttcaatt cccacctatg aatgagaaca |
| 71341 | tgctgtgttt gcttttttgt ccttgcaata gtttactgag aatgatgatt tccagtttca |
| 71401 | tccatgtccc tacaaagcac atgaactcat ccttttttat ggctgcatag tattccatgg |
| 71461 | tgtatatgtg ccacattttc ttaatccagt ctatcgttgt tggacatttg ggttggctcc |
| 71521 | aagtctttgc tattgcgaat aatgccacaa taaacatacg tgtgcatgtg tctttatagc |
| 71581 | agcatgattt atagtccttt gggtatatac ccagtaatgg gatggctggg tcaaatggta |
| 71641 | tttctagttc tagatcccta ggaatcgcca cactgacttc cacaatggtt gaactagttt |
| 71701 | acagtcccac caacagtgta aaagtgttcc tatttctcca catcctctcc agcacctgtt |
| 71761 | gtttcctgac tttttaatga ttgccattct aactggtgtg agatggtatc tcattgtggt |
| 71821 | tttgatttgc atttctctga tggccagtga tgatgaacat tttttcatat tttttggctg |
| 71881 | cataaatgtc ttctttcgag aagtgtctgt tcgtgtcctt cacccacttt ttgatgggat |
| 71941 | tgtttgtttt tttcttgtaa atttgagttc attgtagttt ctgaatatcg gccctttgtc |
| 72001 | agaagagaag gttgcgaaaa ttttctccca ttttgtaggt tgcctgttca ctctggtggt |
| 72061 | agtttctttt gctgtgcaga agctcttttg tttaattaga tcccatttgt caattttggc |
| 72121 | ttttgttgcc attgcttttg gtgttttaga cctgaagtcc tttcccatgc ctatgtcctg |
| 72181 | aatggtattg cctaggtttt cttctagggt ttttatagtt ttaggtctaa catgtaagtc |
| 72241 | tttaatccat cttgaattaa tttttgtata aggtgtaagg aagggatcca gtttcagctt |
| 72301 | tctacataag gctagccagt tttcctgaca ccatttatta aatagggaat ctttccccat |
| 72361 | tgcttgtttt tgtcaggttt gtaaaagatc agatagttgt agttatgtgg cattatttct |
| 72421 | gagggctctg ttctgttcca ttgatctatg tctctgtttg gtaccagtac catgttgttt |
| 72481 | tggttactgt agtcttgtac tatagtttga agtcaggtag cgtgatgcct ccagctttgt |
| 72541 | tcttttggct taggattgac ttggcgatgc aggctctttt ttggttccat atgaacttta |
| 72601 | aagtagcttt ttccaattct gtgaacaaag tcattggtag cttgatgggg atggcactga |
| 72661 | atctataaat taccttgggc agtatggcca ttttcatgat attgattctt ccaacccatg |
| 72721 | agcatggaat gttcttctat ttgtttgtat cctcttttat ttcattgagc agtgttttgt |
| 72781 | agttctccat gaagaggtcc ttcacgtccc ttgtaaggtg gattcctagg tattttattc |
| 72841 | tctttgaagc aattgtgaat gggagttctc tcatgatttg gcactctgtt tgtctattat |
| 72901 | tgttgttcaa gaatgcttgt gatttttgta aattgatttt gtatcctgag acttttctga |
| 72961 | atttgcttat cagcttaagg agattttggg ctgagaaaat gggtttttct agatatacaa |
| 73021 | tcatgtcatc tgcaaacagg gacaatttga cttcctcttt tcataattga atagcctttg |
| 73081 | tttccttctc ctgcctgatt gccctggcca gaacttccaa cagtatgtgg aataggagtg |
| 73141 | gtgagagagg gcaaccctgt cttgtgccag ttttcaaagt gaatgcttcc agtttttgcc |
| 73201 | cattcagttt tctattgact gtgggtttgt tttagatagc tcttattatt ttgagatacg |
| 73261 | tcccatcaat acctaattta ttgagagttt ttagcatgaa gaattgttga attttgtcaa |
| 73321 | aggccttttc tgcatctatt gagataatca tctcgttttt gtctttggtt aggttgatat |
| 73381 | gctagattac atttattgat ttgcatatgt tgaaccagcc ttgcatgaca gggatgaagc |
| 73441 | ccacttgatc atggtggata agctttttga tgtgctgctg gatttggttt gccagtattt |
| 73501 | tattgaggat ttttgcatca atgttcatca aggatattgg tctaaaattc tctttttttg |
| 73561 | gttgtgtctc tgccaggctt tggtatcagg atgatgctgg cctcataaaa tgagttaggg |
| 73621 | aggattccct ctttttctat tgattggaat agtttcagaa ggaatggtac cagttcctcc |
| 73681 | ttgtacctct ggtagaattc ggctgtgaat ccatctggtc ctggactctt tttggttggt |
| 73741 | aagctattga ttattgccac aatttcagag cctgttattg gtctattcca agattcaact |
| 73801 | tcttcctggt ttagtcttgg gagagtgtac gtcttgagga atttatccat ttcttctaga |
| 73861 | ttttctagtt tatttgtgta gaggtgtttg taatattctc tgatggctga ttgtatttct |
| 73921 | gtgggattgg tggtgataac ccctttatca ttttttattg catgtatttg attcttctct |
| 73981 | cttatcttct ttattagtct tgctagtggt ctatcaattt tgttgatctt ttcaaaaaat |
| 74041 | cagctcctgg attcattaat tttttgaagg gttttttgtg tctctatttc cttcagttct |
| 74101 | gctctgattt tagttatttc ttgccttctg ctagcttttg aatgtgtttg ctcttgcttt |
| 74161 | tctagttctt ttaattgtga tgttagggtg tcaattttag atctttcctg ctttctcttg |
| 74221 | tggtcattta gtgctataaa tttccctcta cacactgctt tgagtgtgtc ccagagattc |
| 74281 | aggtatgttg tgtctttttt ttcattggtt tcaaagaaca tctttatttc tgccttcatt |
| 74341 | tcattatgta cccagtagtt attccagaac aggttgttca gtttccatgt agttgagcag |
| 74401 | tcttgagtga gtttcttaat cctgagttct agtttgattg cactgtggtc tgagagacag |
| 74461 | tttgttatga tttcttttct tttacatttg ctgaggagag ctttacttcc aagtatgtgg |
| 74521 | tcaattttgg aataggtgtg gtgtggtgct gagaagaatg tatattctgt tgatttgggg |
| 74581 | tggagagttc tgtagatgtc tattagatct gcttggtgca gagctgagtt caattcctgt |
| 74641 | gtacccttgt taactttctg tttcattgat ctgtctaatg ttgacagtgg gttgtgaaaa |
| 74701 | tctctcatta ttattgtgtg ggagtctaag tctctttgta ggtcactcag gacttgcttt |
| 74761 | atgaatctgg gtgctcctgt attgggtgca tatatattta ggatagttag ctcttctcat |
| 74821 | tgaattgatc cctttaccat tatgtagtgg ccttctttgt ctgttttgat ctttgttggt |
| 74881 | ttaaggtgtg ttttatcaga gactaggatt gcaacccctg cctttttttg ttttccattt |
| 74941 | gcttggtaga tcttcctcca tccttttatt ttgagcatat gtgtgtctct acacgtgaga |
| 75001 | tgggttcctg aacacagcac attgatgggt cttgactctt tatccaattt gccagtctgt |
| 75061 | gtcttttaat tggagcattt agtccattta catttaaagt taatattgtt atgtgtgaat |
| 75121 | ttgatcctgt cattatgatg ctagctggtt attttgctcg ttagttgatg cagtttcttc |
| 75181 | cttgtctcaa tgatctttac attttggcat gattttgcag tggctggtac tggttgttcc |
| 75241 | tttccatgtt tagcgcttcc ttcaggagct cttttaggtc aggcctggtg gtgacaaaat |
| 75301 | ctctcagcat ttgcttgtct gtaaagtatt ttatttctcc ttcacttatg aagcttagtt |
| 75361 | tggctggata tgaaattctg ggttgaaaat tcttttcttt aagaatgttg aatatcggcc |
| 75421 | ccctctctct tctggcttct agagtttctg ccaagagatc cgctgttagt ctgatgggct |
| 75481 | tccctttgtg ggtaacccga cctttctctc tggctgccct taacattttt tccttccttt |
| 75541 | cacctatggt gaatctgaca attatgtgtc ttggagttgc tcttctcgag gagtatcttt |
| 75601 | gtggcattct ctgtatttcc tgaatctgaa tgttggcctg ccttgctaga ttggggaagt |
| 75661 | tctcctggat aatatcctgc agagtgtttt ccaacttggt tccattctcc ccgtcagttt |
| 75721 | caggtacacc tatcagacgt acatttggtc ttttcacgta gtcccatatt tcttggaggt |
| 75781 | ttgttcgttt ctttttattc ttttttatct aaacttccct tcttgcttca tttcattcat |
| 75841 | ttcttcttcc atcacttata ccctttcttc cagttgatca catcggctcc tgaagcttct |
| 75901 | gcattcttta cgtagatctc aagccttggc tttcagctcc atcagctcct ttaagcactt |
| 75961 | ctctgcattg gttattctag atatacattc gtctaaattt ttttcaaagt ttttaacttc |
| 76021 | tttgcccttg gtttgaattt cctcctgtag ctcagagtag tttgatcgtc tgaagccttc |
| 76081 | ttctctcaac tcatcaaagt cattctccac ccagcttttt tccattgctg gtgaggaact |
| 76141 | gcgttccttt ggaggaggag aggtgctctg ctttttagaa tttccaattt ttctgctgtg |
| 76201 | ttttttcccc atctttgtgg ttgtatctac ttttggtctt tgatgatggt gatgtacaga |
| 76261 | tgggtttttg gtgtttatgt cctttctgtt tgtaagtttt ccttctaaca gacaggaccc |
| 76321 | tcagctgcag gtcttttgga gttttctaga ggtccactcc agaccctgtt tacctgggta |
| 76381 | ttagcagcgg tggctgcaga acagcagatt ttcatgaacc gcgaatgctg ctgtctgatc |
| 76441 | gttcctctgg aagttttgtc tcagaggaga acccggccat gtgaggtgtc agtctgcccg |
| 76501 | tactgggggg tgcctcccag ttaggctgct tgggggtcag gggtcaggga cccacttgag |
| 76561 | aaggcagtct gcctgttctc agatctccag ctgtgtgctg ggagaaccac tgctctcttc |
| 76621 | aaagctgtca gacagggaca tttaaatctg cagaggttac tgctgtcttt ttgtttgtct |
| 76681 | ctgccctgcc cccagaggtg gagcctaaag aggcaggcag gcctccttga gctgtggtgg |
| 76741 | gctccaccca gttcgagttc ctggctgctt tgtttaccta cgcaagcctg ggaaatggcg |
| 76801 | agcgaccctc cccaagcctt gctgccgcct tgcagtttga tctcagactg ctgtgatagc |
| 76861 | aatcagcgag actccgtggg cgtgtcaccc tctgagccag gtgcaggata taatctcctg |
| 76921 | gtgcgccgtt tgttaagacc atcggaaaag cacagtatta gggtgggagt tacccgattt |
| 76981 | tccaggtgcc gtctgtcacc cctttctttg actaggaaag ggaactccct gaccccttgc |
| 77041 | acttgccgag tgaggcagtg cctcaccctg cttcggctca tgaactgtgc actgcaccca |
| 77101 | ctgtcctgca tctgctctct ggcactccct agtgagatga acctggtacc tgagatggaa |
| 77161 | atgcagaaat cacccatctt ctgcattgct gacactggga gctgtagacc ggagctgttc |
| 77221 | ctattcggcc atcttggcta cacttggtag tttcttttcc ctctgcttct gggttctgag |
| 77281 | agtttgtcac taacatagga ttccagaaca ctgctgcagg gttctgagtg attgttgctc |
| 77341 | acatgggatt caaaaacact cctgctgggt tcagagtgtt atccctcaca tacgattcca |
| 77401 | gaacactgct atgaggttct gaatgtttgt ccttcacaaa ggattccaga acactgctgc |
| 77461 | tgggttctga gtgtttgtcc ctcatatagg attccagaac actgctgctg ggttctgagt |
| 77521 | gtttgtccct cacataagat tccagaactc tgctatgaag ttctgaatgt ttgtggctca |
| 77581 | cataggattc cagaacactg ctgctgggtt ctgagtgttt gtccctcaca taggattccg |
| 77641 | gaacaatgct gctgggttct gagtgcttgt cccttatatt ggattccaga acaatgttat |
| 77701 | gagggtctga atgtttttcc ctcatgtagg attcaagaac actgctaaga gggtctcaat |
| 77761 | gtttttccct cacaaaggat tgcagaactc tgctgctggg ttctgagtgt ttgtccctga |
| 77821 | tataggattc cagaacactg ctatgagggt ctgaatgttt ttccctcaca aaggattcca |
| 77881 | gaacgctgct gggttctgtt tgtttgtccc ccacaaagga ttccagagca ctgctgctgg |
| 77941 | tttcttagtg tttgttccac acatgattcc agaacacttc tgcgatggtt tgaatatttc |
| 78001 | tccctcagat aggattccag tacacagtgg ctgggttctg agtgttggtc cctcacatag |
| 78061 | gattccagaa cactgcttct gggttctgag tgtttgtcac tcacatagga ttccagaaca |
| 78121 | ctgcttcgag ggtctgagtg tttgtccctc acaaaggatg ctggaacact gctgctggtt |
| 78181 | tctgagtgtt tgtcactcac agaggattcc agaacactgc tgcttccaga acactgctgc |
| 78241 | tgggttctga gtgtttgtcc ctcacatagg attccagaaa actgctatga gtgtctgaag |
| 78301 | gtttgtccat aacaaaggat tccagaacaa tgctgctgga tctgaatgtt tgtccctcac |
| 78361 | acaggattcc aggacactgc ttcgagggtc tgagtgtttg tccctcacac aggattccag |
| 78421 | accactgctt ctgggttctg agtgtttgtt ccacatatag gattccagaa cacagctaca |
| 78481 | aggctatgaa agtttgtctc tcacaaagga tttcagaaaa atgctgctgg gttctgagtg |
| 78541 | tttgttcctc acataggttt ccagaaaact gctgctggga tctgagggat tgtccctgtc |
| 78601 | attgagtacg agaacactgc tgctgggttc tgaaggtttg tccttcactt agaattgcag |
| 78661 | aatactgctg ctaggttatg agggtttgtc cctcatgtag aattctagaa ctctgctgct |
| 78721 | gggttctgag catttctcta tcacatgaga ttctggaaca ctgctaaaat gtaggaatgt |
| 78781 | ttgtccctca caaagagtcc agagcaccgc ttgtgggttc tgagtgtttg tccctcacat |
| 78841 | aggattgcag aacacagctg ctaggttctg agtgtttctc ccaaacatag gattccaaaa |
| 78901 | caccgctcag agggtctgaa agtttcttcc tcaccaagga ttccagaaca ctgctgctgg |
| 78961 | gttctgaaag tttgtccctc acatacgatt ccagaacact gctatgaggc tctgaatgtt |
| 79021 | tgtccctcaa agggattcca gaacactgct tttgagttct gagtgtttgt ccgacacaaa |
| 79081 | ggattccaga acactgctgc tggcttctga gtgtttgtcc ctcacatagg atttcagaac |
| 79141 | actgctatga gggtctgcat gtttttccct cagaaagcat tctgtatcac tgctacgagt |
| 79201 | gtctgaatgc ttgtccctca cataggattc cagaacactg ctactgggtt ctgactgttg |
| 79261 | gtccttcaca taggattcca gaacactgct ccgagggtct gaatgtttgt ccctcacata |
| 79321 | ggattccaga acatttgctg ctggtttgtg agtgtttgtc cctcatatgg gattccagaa |
| 79381 | cacttctgct gggttctgtt tgtccctcat ataggatacc agaacactgc tattggcttc |
| 79441 | agagtgtttg tccctcacat aggattccag aaaacttcta agaggttccg aatgttttcc |
| 79501 | ttcagatagg attccagaac actgctgctg ggttctgaga gtttgtccct cacataggat |
| 79561 | tccagaacac tgctaagagg gtctgaatgt ttgttcctca gataggattc caggacactg |
| 79621 | ctgctgggtt gtgtgtgttt atcactcaca tagaattcca gaacactgct acaagtgtct |
| 79681 | caatgtttgt ccctcacata gaattccaga atattcctgc tgtggtctga atgtttgtcc |
| 79741 | ctcacatatg attccaggac gctgctgctg tgttctgcgt gtttgtcccc cacataggat |
| 79801 | tcaagaacac tcctgctgtc ttctgaacgt gtgtccttca cagaggattc cagaacactg |
| 79861 | ctactagcct ctgaattgtt gtcccccaca gaggattcca gaacactggt actagggtct |
| 79921 | gaatgtgagt ctctcacata ggattccaga acactaatgc cggggtctga atgtttgtcc |
| 79981 | ctcacctagg attgcagaac actgctacga ggttctgaat ctttgtccat cacgtagcat |
| 80041 | tatagaacac tgctacgaag ttctgaatgt ttttccctca gagaggatca aagaacactg |
| 80101 | ctgctagtgt ctgaaatttt gatgatcacg taggattcca gaactctcct gctgtggtct |
| 80161 | gtatgtttgt ctctcacata ggattccaga acactgctac gagggtctga atgtttgtcc |
| 80221 | ctcacaaagg attctagaac actgctgctg ggttctgagt gattgtccct cacataggat |
| 80281 | tccagaacac tgctgctggg ttctgagtgt ttgtccctca cataggattc cagaacactg |
| 80341 | ctacaagggt ctgaatgttt atccctcaca aagggttcca gaacactgtt actggattct |
| 80401 | gagtgtttgt ccctcatatt ggattccaga acaatgctac gagggtctga atatttttcc |
| 80461 | ctcacatagg attcgaaaac actcttacga gggtgtgaat gtttttccct cccaaaggaa |
| 80521 | tccagaacac tgctgctggg ctctgaatgt ttgtccctca tataggattc cagaacactg |
| 80581 | ctacgaggat ctgaatgttt tcccttacaa aggattgtag aacactgcta ctgggttctg |
| 80641 | tttatttgcc cctcacaaag gattccagag cactgctgca tatttcttag tgtttgcccc |
| 80701 | tcacatagga ttccagaaca cttctccaag gttctgaatg tttgccctca gataggattc |
| 80761 | cagtacactg gctggattct gagtgtttgt ccctcacata ggattccaga tcactgctac |
| 80821 | aggttctgaa tgtttgtccc tcacaaagga ttctagaaca ctgctactgg tttctgagtg |
| 80881 | tttgtccctc acaaaggatt ctagaacatg gcttctgggt tctgtgtgtt tgtccctcac |
| 80941 | ataggatgcc aaaacactga tgctgggttc tgagtgtttg tccctcacat agcattccag |
| 81001 | aacactgctg ctggcttctg agtgtttgtc cctcacatac aattccagaa cactgctacg |
| 81061 | aagttctgaa ggtttgtcgc tgacatagga ttccagaaca ctgctgctgg gttctgagtg |
| 81121 | tttgtccctc acataggatt ccagaacact gctacaaggg tctgaatgtt tatccctccc |
| 81181 | aaaggattcc agaacactgc tgctgggttc tgagtgtttg cccctcaaat aagattccag |
| 81241 | aacactgcta tgagggtctg aatgtttttc cctcacatag gattcaaaag actgttacga |
| 81301 | gggtctgaat gtttttccct aacaaaggat tgtagaacac tgtcactggg ttctgtttgt |
| 81361 | aggtccctcc cataggattc cagaacacta atacgagggt ctgaatgttt gtccctcaca |
| 81421 | taggattcca gaacagagct gctgtgtcct gattgcttgt ctctcacaaa ggattccaga |
| 81481 | acactgatgc tgggttctga gtgttcgtct atcacatagg attccagaac aaagctgctg |
| 81541 | ggtcctgatt gtttgcccct cacaaaagat tacaaaacac tgctacgagg gtctgaatgt |
| 81601 | ttgtccctca tataggattc cagatcactg ttgctgggtt ctgagtgttt gtccctcaca |
| 81661 | taggattcca gaacactgct gctgggttct tcgtgtttgt acctcatata ggattccaga |
| 81721 | gcactgctac aagactctga atgcttgtcc cttacatagg attccagaac actgttttga |
| 81781 | gagtctcaat atttgtccct cacaaaagat tgcagacaac tgctgctgag ttctgagagt |
| 81841 | ttgtccttca cttaggaatc cactgattct gggttctgaa tatttgtcct tcacatagga |
| 81901 | ttccagaaca gtgctgctgg cttctgagtg attgtcccgc acgtaggatt ccagaacact |
| 81961 | gttacgaggg tctgaatgtt cttccctcac aaaggcttcc agaacactgc tgctggtttc |
| 82021 | tgtttgtttg tccctcacaa aggattccag agcactgctg ctggttattt agtgtttgtt |
| 82081 | gcccacatag gattccagat cacttctgcg aaggtctgaa tgtttagccc tgagatagga |
| 82141 | ttccagtaca cactggctcg gttctgagtg tttgtaccac acactggatt ccagaacact |
| 82201 | gctgctgggt tctgagtgtt tgcccctcac ataggattcc agaaaactgc tatgaggacc |
| 82261 | ttaatgtttg tccatcacaa aagattctag aacaatgctg ctggatctga gtgtccctca |
| 82321 | cataggataa cagaacactg cttcgagagt ctaaatgttt gtccctcaca aaggattcta |
| 82381 | gaacattgct gctggtttct gagtgtttgt cactcacata ggattccaga acactactgc |
| 82441 | tgagttctga ggctttgtat ctcacatagt atttcagaac actgctatga ggttctgaat |
| 82501 | gtttgaccct cacagagcat tgcagaacag ggctatgggg atctgaatgc ttgtccctca |
| 82561 | catatgaatc cagaacactg ctgcagggtt cagagtgttt atcccacaca taggattaca |
| 82621 | gagcactgtt ctgagcatct gagtgtttga ccctcacaaa ggattgcaga acactgctgc |
| 82681 | tgggctctga gtgtttgtcc cttacatagg attctagaac actgctgctg ggttctgagt |
| 82741 | gtttgtctct cacataggat tccagaacac ttctctgagg atctgaatgt ttgtccctca |
| 82801 | caaaacattc cagaacactg ctgctgggtt ctgagtgttt gtccatcaaa taggattcca |
| 82861 | gaacacggct gatgggctct gtttgtttgt ccctcacaaa ggattccaga gcactgctgc |
| 82921 | tggtttctga atgtttgtcc ctcacatagg attccagaac acttctacga ggctccgaat |
| 82981 | gtttgtcctt cagataggat tccagaacac agtggatgtg tgatgagtgt ttgtccctca |
| 83041 | aataggattc cacaacactg ctttgagggt ctgaatgttt gtatctcaca aaccagtcta |
| 83101 | aaacactact tctgggttct gagtgtttgt ccctcacata gaattctaga acactgcagc |
| 83161 | tcgtttctga gtgtttctcc gtcactttgg attccagaac actgctaaga ttgtctgaat |
| 83221 | gttcgtccct taccaaatat tccagaacag tgctgctggg ttctgagtgt ttggccctca |
| 83281 | cattggtttc cagaacactg ctgtgattgt ctgaatgttt gtccctcaca aattattcca |
| 83341 | gagtactgct gctgggttct gagggtttgt ctctcacata gaattaaaga atactgctgc |
| 83401 | tgagttctga gattttgtac ctcacatatg attccagaac actgctatga gggtctgaat |
| 83461 | atttgttcct cacagagcat tccagaatgg tgctatgagg gtctgaatgc ttgtccctca |
| 83521 | cataggcagg gttctgagtg tttgtccctc acacaggact ccagaacact gctctgagag |
| 83581 | tctgagtgtt tgaccctcac aaaggattcc agaacactgc tgctgggttc tgagtgtttg |
| 83641 | tcccacacat aggattccag aacactgctg ctgctttctt agtgtttgtc tctcacatag |
| 83701 | gattccagaa cactgctgcg aggatctgaa tgttgtccct cacaaaggat tagagaacac |
| 83761 | tgctgctgag ttctgagtgt ttgtccctct cattggattc cagaacactg ctgctaggtt |
| 83821 | ctgagggttt gtccctcaca taggattcca gaacactgct gctggtttct tgggtttgtc |
| 83881 | tctcacatag gattgcagaa cactgctacg attatctgaa tgttgtccct cacaaagtat |
| 83941 | tacagagcac tgctgctggg ttctctctgt ttgcccctca cattggatta cagaacactg |
| 84001 | ctgctaggtt tgagggtttg tccctcacat aggattccaa aacactgttg ctgggttctg |
| 84061 | agtgtttgtc cctaacatac gattccagaa cactgctatg attgtctgaa tgtttgtccc |
| 84121 | tcacaaagta ttccagagca ctcctcctgg gttaagagtg tttgcccttc acataggatt |
| 84181 | ccagaacagt gctgctgggt tctaagtgtt tgtccctcac ataggattcc agaacactgc |
| 84241 | tacgaggatc tgaatgtttg tccctcacaa attattccag agtactgctg ctgggttctg |
| 84301 | agtgtttgtc cctcacatag gattccagaa cactgatacg aaggtctgaa tgtttgtccc |
| 84361 | tcagatagga ttacagaaca cagctacgag gttctgaatg attgtccctc acataggatt |
| 84421 | ccagaacaca gtggctgggt tctgagtgtt tgtccctcat ataggatttc agaacactgc |
| 84481 | tatgaatttc tgaatgtttg tcgctcacag aggattctag aactctgcgg ctgggttgtg |
| 84541 | ttttcccccc acataggatt ccagaatact gctgctgggt tctgagtgtt tgtccctcac |
| 84601 | ataggattcc agaactctcc tgctgggttc tgtttgttta cccctcacaa aggaatccac |
| 84661 | agcactgttg ctggtttctg agtgtttgtc cctcactcag gataccagaa cactgctacg |
| 84721 | atggtctgaa tgtttgtccg tcacaaagga ttcataacac tgctatgggt tctgactgtt |
| 84781 | tgtccctcac aatggattcc agaaaactgc tttgagagtc tcaatgtttg tccctcacaa |
| 84841 | agtattccag agcactgctg cggggttctg tgtgtttgtc cctcacatgg gattcaaaaa |
| 84901 | cactcctgct gggttcagag tgttttccct cacataaaat tccagaacac tactacaagg |
| 84961 | ttctgaatgt ttgtccctca cataggattc cagaacactg ctgctgggtt tcgagtgttt |
| 85021 | ctccctcaca taggattcca gaacactgct gctcggttct gagtgtttgt ccctcacata |
| 85081 | gcattccaga acacaggtac gaagttctga atgtttgtag ctcaagtagg attccagaac |
| 85141 | aatgctgctg ggttctgaat gtttgacact cacacaggat tccagaacac tgctgctggg |
| 85201 | ttctgagtgt ttgtccctca cataggattc cagaacactg cttcgagggt ctgaatgttt |
| 85261 | ttccctcaca aaggactcca gaacactgtt gctgggttct gagtgtttgc ccctcatata |
| 85321 | ggattccaga acaatgctac gagtatctga atgtttttcc ctcacatagg attccagaac |
| 85381 | attgctacga gggtctgaat gtttttccct cacagagtat tccagaacac tgctgctggg |
| 85441 | ttctgaatgt ttgtccctca cataggattc cagaacactg cagctgggtt ctgagtgttt |
| 85501 | gtccctcact ttggattcca gaaaacgtct acgacggtct gaatttttgt ccatcacaaa |
| 85561 | ggattttaga acactgctgc tggatctgag tgtttgtccc tcacacagga ttccagaaca |
| 85621 | ctgcttcgat ggtctgaatg tttgtccctc acaaaggatt ctagaacact gctgctggtt |
| 85681 | tcttagtgtt tgtcactcac ataggattcc agaacactgc tgctgggttc tgagtgtttg |
| 85741 | tcctcacata cgattccaga acactgctat gaggttctga atgtttgtcc ctcacaaagg |
| 85801 | attccagaac actgcttctg agttctgagt gtttgtctgt caaataggat tccagaacac |
| 85861 | tgctgctggg ctctgtttgt ttgtccatca caaaggattc cagaacactg ctataggttt |
| 85921 | ctgagtgttt gtccctcaca taggattcca gaacaattct acgaggctcc gaatgtttgt |
| 85981 | ccttcagata ggatttcaga acacagtggc tgggttctga gtgtttgtcc ctcacatagg |
| 86041 | gtatcagaac actgctgctg ggatctgagc gtttgtcttt cacagaggat tccagaacac |
| 86101 | tgctgctttg ttctgagtgt ttgtccctca cataggattc cagaacactg ccgctcggta |
| 86161 | ctgagggttt gtcccttaca tagaattcta gaacactgca gctcatttct gagtgtttgt |
| 86221 | ccctcactta ggattccaga acaatgctac gattgtctga atgtttgttc cttaccaagt |
| 86281 | attccagaac actgctgctg ggttctgagt gtttggcact cacattggtt tccagaacac |
| 86341 | tgctacgagg gtctgaaaat ctcgcacata ggattccaga acactgctaa gatggtctga |
| 86401 | atgtttttcc ctcagaaagg attctggaac cctgctactg gattctgttt gtttgtctct |
| 86461 | tacaaaggat ttcagagcac tgctcctggt tgctgagtgt ttgtccctca caaaggattt |
| 86521 | cagagcactg ctcctggttg ctgagtgttt gtccctcaca taggattcca gaacacttct |
| 86581 | acgagtgtct gaatgtttgt ccctcagata ggattccaga agacagtgga tgggttctgg |
| 86641 | gtgtttgtcc atcacatagg attccagaac actgctgctg ggttctgagt gtttgtccct |
| 86701 | cacataaggt tccagaacac tgcttctgag tgtttgtccc tcacatagga tcccagaaca |
| 86761 | ctgttgctgg gttctgagta tttgtccctc tcatgggatt ccggaaacct gctgctgagt |
| 86821 | tcagagtgtt tttccctcac ataggattcc agaacactgc tgctgggttc tgagtgtttg |
| 86881 | tccctcacat tggattccag aacactgctg ctgggttttg agtgtttgtc cctcacatag |
| 86941 | aattc |

An exemplary human KRT8P8 nucleic acid sequence is set forth below (SEQ ID NO: 168; GenBank Accession No: NG_009749.1, Version 1, incorporated herein by reference):

```
      1 acccacagaa tcaaaaaact cagtgaatca taaacaggat aaataaaaag aaaaccacac
    61 ctaggcacat catagtcaag ttgttaaaaa aaacaaagag accaagaagc agcttctctg
  121 ctccttctgg aatctctgcc tggttcagcc cacctgcctc cactcctgcc tccaccatgt
  181 ccatcagggt gacccagaag tcgtacaagg tgtccacctc tggcacacgg gccttcagca
  241 gccgcttcta cacgagtggg cccggtgccc gcatcagctt ctccagcttc tcccgagtgg
  301 gcagcagcag cttctggggt ggcctgggcg gaggctatgg tggggccagc ggcatcggag
  361 gcatcaccgc cgtcatggtc aaccagagtc tgctgagccg ccttaacctg gaggtggacc
  421 ccaacatcca ggccgtgcgc acccaggaga aggagcatat caagacccta ggcaaatttg
  481 tctccttcat cgacaaggta ccgttcctgg agcagcagaa caagatgctg gagaccaagt
  541 ggagcctcct gcagcagcag gagatggctc agagcaacat ggacaacatg ttcgagagct
  601 acatcaacaa ccttaggcgg cagctggaga ctctgggcca ggagaagctg aagctggaga
  661 cggagcttgg caacattcag gggctggtgg aggacttcaa ggacgagaat gaggatgaga
  721 tcaatatgcg taaagagatg gcaaatgaat ttgtcctcct caagaaggat gtggatgaag
  781 ctgacatgaa caaggtagag ctggagtctc gcctggacgg gctgactgac gagatcaact
  841 tcctcaggca gctgtatgaa gaggagatcc gggagctgca ttcccagatc tcggatacgt
  901 ctgtggtgct gtccgtggac aacagccctc cctggacatg gaaagcatca tcgctgaggt
  961 caaggcgtag tacgaggtga tcgccaaccg cagcctggct gaggctgaga gcatggacca
1021 ggtcaagtat gaggagctgc aggtgctggc tgggaagcag ggggatgacc tgcggcatac
1081 agactgagat ctccgagatg aaccggaaca tcagctggct ccaggctgag actgagggcc
1141 tcaaaggccg gatggcttcc ctgtggaggc cgccatcgca gatgccgagc agcttgggga
1201 gctggccgtt aaggatgcta acgtcaagct gtccgagctg gaggccaccc tgcagcgggc
1261 caagcaggac atggcgaggc ggctgcgtga gtaccaggag ctgatgaacg tcaagctagc
1321 cctggacatc gagatcgcca cctacgggaa gctgctggag ggcgaagaga gccagctaga
1381 gtctgggatg cagaacatga gtattcatat gaagaccacc agcggttttg caggtggtct
1441 gagctcggcc tatgggggcc tcacaagccc cggcctcagc tacggcctgg gctccagctc
1501 tggctctggc gcgggcttca gctccttcag ccgcaccagc tccaccaggg ccccggttgt
1561 gaagaagatc gagacccgcg atgggaagct ggtgtccgcg tcctctgacg tcccgcccaa
1621 gtgaacagct gcggcagccc ctcccaccct gcccctcctg cgacttgccc agagcccggg
1681 agggaggccg ctgtgcaggg gagcacaggg aacagaagac acacctgagg ctcggctcta
1741 gccctcagcc caccctcggc ggaattcact gcctgaggac cacccttgcc catgcctcca
1801 actacaaaac aattcaattg cttt
```

An exemplary human miR218-1 nucleic acid sequence is set forth below (SEQ ID NO: 169; GenBank Accession No: NR_029631.1, Version 1, incorporated herein by reference):

1 gtgataatgt agcgagattt tctgttgtgc ttgatctaac catgtggttg cgaggtatga
61 gtaaaacatg gttccgtcaa gcaccatgga acgtcacgca gctttctaca

An exemplary human XIST nucleic acid sequence is set forth below (SEQ ID NO: 170; GenBank Accession No: U50908.1, Version 1, incorporated herein by reference):

```
      1 aagcttggct cccttgaggt taggaattcg ataccagcct ggcaagcatg gtgaaccccg
    61 tctctgctaa aaatacaaaa attagccagg catggtggca cacgcctgta atcccagcta
  121 ctcgggaggc tgaggcagga gaatcacttg aacccgggag gcagtggttg tagtgagccg
  181 agattgtgcc actgcactct ggcctgggct acagagtgag actctgcctc aaacaaaaaa
  241 caatcaaaca aaaaccaaat agcaaacaga tatatgaaaa agtgctcaat atcaataatc
  301 atcacaaaaa tgcacatcag gctgggtgtg gtggctcaca cctataatcc cagcactttg
  361 ggaggccaaa gcgggtggat cacttgaggt caggagttca agaccagcct gcccaatata
  421 gtgaaacatc ttctctgcta aaaatacaaa aattagctgg gcatggtggt gggtgcctgt
  481 aatcccaccc gctcaggagg ctgaggcagg aggatcactt gaacccggga ggcagaggtt
  541 gcaatgagcc gagatggcac cactgcacgg gctgtaacct gggcaacaga atgaggctgt
  601 gtctgaaaac agaaataaat aaggccaggc gcagtggttc acatctgtaa tcccagtgtg
  661 attacagatc acactgtggg aggccaaggc aggcagatca cctgagctca ggagtttgag
  721 accagcctgg tcaacatggt gaacccccgt ctctattaaa aatatacaaa ttagctgggc
  781 atggtggcag atgcctataa tccaagctac ttgggaggct gaggcatgag aattgcttga
  841 acccaagaag tggaggttgc agtgaaccgt gatcacacca ctgcactcca gccttggcga
  901 cagagagaga ttctgtctca aaaaaataaa aattaaaaaa aaaaaagaaa gaaagaaaaa
  961 aaaaagaaaa atgcaaatca aaaccacaat gcaatattgc ctcacacctg ttagaatggc
1021 tattttcaag aagacaaaag ttaaggattg gtgagggtgt ggagaaaagg gaacatttat
1081 acagtgttgg tggaaatata aattagttca accatcatgg aggcttctga aaacactaaa
1141 aatacaacta ccttatgatc caataatccc acttgtgggt gttgttcaaa gaaattgaaa
1201 tcagtgtgtt gaagagacat ttgtcctcct atgtttatta tagcattatt cacaatagtc
1261 aagatatgaa atcaaggtaa gtccccatca acagatgaat gtataaagaa aatgtggtat
1321 acatacacaa tggaacacta gtcagcctta aaaagaagga aaatctgtca tttgcaacaa
1381 catatatgaa cctggaggac attatgctaa gccaggcaca gaaagatgaa tactatatta
1441 tctcacttgt atgtgtaatt ttcaaaactt gaacttgtag ggagtagact ggtggttaat
1501 aatctgaagg gagggtaaaa atgtgttgat caaaggataa aaattttagt tagagagaag
1561 gaataagttt ttgagatata ttgcagagca tgatgacaat agtgataagg tattgtacat
1621 ttcaaaaact gctaagtaaa ttttaagtat tctcatcaca aaataagtat gtgagctgag
1681 atatatagta attagcttga tttaattatt ctacaatgca tgcatatatc aaaataccac
1741 attttactcc atgaatatat acaattattt gtgaactaaa aatacatttt gaaaaagaac
1801 tactagaaaa actaaaaatg agaaaataaa taagcacatt aattaattaa ttaaaatcct
1861 tgtctagtaa gtttaatgtt ggtactttct cagggaccat tttttgttaa ttaccttttt
1921 ttctatatat tagctataat ttcctgtttt ttgtatgcct tgtaattttt aataaaaatg
1981 aacagtttta atattataat gtgatacctt gggaaataaa attcctccct ctccctagtg
2041 tttgctggtg attttttttt ttttttttga gatggagtct tgctctgtca ccaggcttgg
2101 cgcggtctca gctcactgca acctccgcct tccgggttca agcgattctc ctgcctcagc
2161 ctcccgagta gctgggatta caggcgtgtg ccaccatgtg cagcttattt ttgtatttat
2221 agtagagatg gggtttcacc atattgacca ggatggtctt gatttcctga cctcatgatc
2281 tgtctgcctc gcctccaagt gctggattac agcatgagcc accgcacccg ccactggtga
2341 tgttttaata gattgtaatt tttttgaggt agcgtttgct ttctcaccca gtctggagtt
2401 cagtgggcca atcacaagcc cactgtatcc ttgaactact gggctcaatc aatcctcctg
2461 cttcatcctc ctgagaagct agaactacgg gtatgcacca ccacgcctgg ctaattttta
2521 tattttttat ttttgtagag acagaggtct ctctattttg cccaggctga tctcgaactc
2581 ctggcctcaa aagatcctcc tgccttggcc tcctaaagtg ctgagattaa aggtgtgagc
2641 caacgtaccc agcccattta cagtattttt aatgttgacg tgagctatgt atacaattgt
2701 taagcatata ctcactgagc tttaatatgt ataaccccgt gttctctttt gataaactag
2761 aaaacctttc ccaacatcct aatacacttt tgaaggatat cataaatagt gtgtttgagg
2821 gctagaggaa tggcccaagt actgtgatga ggttcatagg atttggggct atttggaaga
2881 cccttacaag gcatatagca caacttttct atctgttgat tgtctctgta gcatcctcac
2941 aaaatccact attggcttgc aggtcttcag agactggcat tcaccagttc tggaggtgtt
3001 aactgttcca tctttggaca gctcagagta ttcagacgtc tttatttatt agaactacac
3061 acaacaggac tattcatcaa cttcccccat tgcagacagc cctttgagtt gtaaagagcc
3121 cttccccagg tcttttattt tcttttccat aatatgtcct ggtcccttta actgcttttc
3181 actggtgatg atttggaggg cccccaccag cctgctaatt agatcctgaa ggctttatca
3241 aagtcctctt taaatggcat agccctgaag ccaaggacag actcctgttt agtgggggga
3301 ggggctcttc aaattagagg gggttgagca cttttctttg accctttctt ctgatactga
3361 ccttgtatga atcagacttt ccccccttta tatcagattc tggtcctgcc ctgtatacaa
3421 ctttggctcc tttcctgcag acaggctcag tgcttctttg gtgattttat gctgccatct
3481 ggtggctaag tcgacagtgc cggctttttt tgggcctaat ttagcagagg agaggatgta
3541 cagatgaaca tagtgacaat cctagaactg gaataatctg aatctttaaa aagaggacct
3601 gatttcttcc tattgtgtgc catgtgattg ggagcccaaa gaagaggatg ctaacacagc
3661 tatggcatgg ccacagaaaa tatgcctgaa atatccccat cagagcccaa ctcttactag
3721 ctaattgact ttgagcaagt cacttttcct tctagtgtct cagtttccat acttctgatc
3781 ctaacaggtt tataaaatgg agccaagcag tagtgaaggt gaggccaggt cgggaagagg
3841 aggaagtaaa gtgaattgaa agctagtgag ggcaggggag taagtaaaac ctgaaggtga
3901 taaatggcag aagagggcag gactgctcta gggcaaaggc ctaggcaaat atttttcaga
3961 agttcctttt aatgtaaaca gtttgattaa aaatatatta caaatacatg ggcccatgtg
4021 aggtgtagtg atttatcaat gaaaatttag aataccaggt ggagaagaga tgcttacgta
4081 tttgtttatt caacagttca catatcaatt tttataatga ccttgcacca tatcttcctt
4141 tttaggtcca gaaatcatct ctttgtgttt attgtcaaat gcaccattcc tggctaattt
4201 cacatctccc tctatgagaa aaacatagca acactgtact acttccattt ttgcaatggc
4261 actctggaac ctctttgtat tgaaattata aatttcttgc ttttgcagtt tctttagcct
4321 aagggtcaac agtggattta ggtaggccac agctctgaac atgtcctgtg tccaatgtga
4381 gtgtcacccc ataacggcac atcagcacca ttttgcatgc ctagccttaa gagttaccat
4441 ttaaaaaact ccaatgacaa ccaatgagag tgatctgcta gcgtggctgc ccacctggaa
4501 ccagggcctg gtcacagaaa cccaggacaa ccagtttata aattgtcttt ttggcttttt
4561 atttgaaaag gtatttttgt gaaacaaaag gttttaattt cagagtcaaa tttatcaatc
4621 tttcatattt gatgctactt gagtctttag aaaatttttc cccaactaaa tttctttttt
4681 ctttttttga gatggagtct tggtctgtcg cccgggctgg agcgcagtgg caccatctcg
4741 gttcactgca acctccgcct cctggattca agtgattctc ctgcctcagc ctcccaagta
4801 gctgggatta taggtgcctg ccaccatgcc cggctaattt ttgtatttat agtagagacg
4861 gggtttcgct atgttggcca ggctggtctc aaactcctca cctcaggtga tccagccacc
4921 tcggcctccc aaagtgctgg gattacaagc atgagccacc gcgcccggcc aaccaaattt
4981 cttaaaagta tagaacattt ccacacttgt agaaacttct agtagataaa gttgtattag
5041 aggtttggta atccagtctt tctgatagct gcttcatagg tgtcactgta ggtagtttct
5101 ctctttgtta agcattgttt tcctcaaagg taccattgta ttgcaccact ttacaataca
5161 ataaccctca tttggaatga caagtcagat tacttttttt gtttgatcca aggacactta
5221 gaagatggaa ggtatttctt aaggaagaaa atttgcatac tttattattt ttctggaaac
5281 tgaagagcaa gcttacctga tagatttggg tcgatagtta cactttccag ttcagccagt
5341 gggagggtaa tgtatccttt gactttctag aagttgctct ataatactta agttgccatt
5401 gtctcccttc tggttctctg ttgtttctcg aatgacatag aagagtttat gagattaatt
5461 gcttcctact gaaatagcca ggtttctgag ggcagctttg gcttttatat gagctttttc
5521 ttcctaccac ttatcagtta ctgacagagg gtcaacttca ctactgtgtc atatggttgg
5581 tccaatgttg ggttcctcag attcatggag gaaataggaa aatagtaaag attaaggagt
5641 catagttaaa aaattacaaa caggtcacaa accagtactc tttcttgatt atttaggaac
5701 caaatagcca ttctatgaaa tgtcttcctt tcctttttct ctcttgctca ccaattgact
5761 cgtaagcact tccgttctct tatggttggg agccatacaa ggtagagtgt tggagtaaaa
5821 actacattga ctctgaatca tggtgtgtga cctcgggcac atgatgtaac ctctatagga
5881 ctctatttta atgtataaaa caggaataat cctttattat tatctatgca atacatattc
5941 cattatctat tacatgggat aatggaatgg gaagtccctt gaagatggta ctaacctcaa
6001 tgtattactc ctttctagct tctttgggtc aaaagtttgg tggaggagtt acaaattctg
6061 gtttgaatga tatatttgga tactttatca acacatcaaa gctctaccta tcccttcccc
6121 cattctcaaa accaagctga attaacatct ttacatttat tatgcagttt atggaggatt
6181 ttagcattaa ttattgcttg atttactcaa tgtccccatg ttatagatga gaactggaaa
6241 acccattgaa gttgtgactc ctggtctaga aatgaagtct acttccagtt aatgttcttt
6301 ctggtatgtc tttgctttct tgaaatttcc cttttttgtc cttactgggt aaattttgaa
6361 ccaaccaaat cacaaagatg tccggctttc aatcttctag gccacgcctc ttatgctctc
6421 tccgccctca gccccccctt cagttcttaa agcgctgcaa ttcgctgctg cagccatatt
6481 tcttactctc tcggggctgg aagctt
```

An exemplary human PSG10P nucleic acid sequence is set forth below (SEQ ID NO: 171; GenBank Accession No: NR_026824.1, Version 1, incorporated herein by reference):

```
      1 gcagaaggag gaaggacagc acagcctaca gccgtgctca ggaagtttct ggaacctagg
    61 ctcatctcca cagaggagaa cacacaagca gcagagacca tggggcccct ctcagcccct
  121 ccctgcacac agcacatcaa atggaagggg gtcctgctca cagcatcact tttaaacttc
  181 tggaacccac ctaccattgc ccaagtcacg actgaagccc agccacccaa agtttccgag
  241 gggaaggatg ttcttctact tgtccacaat ttgccccaga atcttactgg ttacatgtgg
  301 tagaaagggc aaataaggga cctctaccat tacattacat catatgtagt agacggtcaa
  361 agaattacat atgggcctgc atacagtgga cgagaaacag tatattccaa tgcatccctg
  421 ctgatccaga atgtcacccg ggaggacgga gtatcctaca ccttacacat catacagcga
  481 ggtgatggga ctagaggagt aactggaaat ttcaccttca ccttataccc gaagctgccc
  541 aagccctaca tcaccatcaa caactcaaaa cccagggaga ataaggatgt cttacccttc
  601 acctgtgacc ctaaaagtga gaactacacc tacacgtggt ggctaaatgg tcagagcctc
  661 ccagtcagtc ccagggtaaa gtgacccatt gaaaacagga tcctcattct acccagtgtc
  721 acgagaaatg aaacaggacc ctatcaatgt gaaatacggg accgatatgg tggcatccgc
  781 agtaacccag tcaccctgaa tgtcctctat ggtccagacc tccccagaat ttacccttca
  841 ttcacctatt accgttcagg acaaaacctt tacttgtcct gcttcgcgga atgtaaccca
  901 ccggcatagt actcttggac aattaatggg aagtttcagc aatcagaaca aaagctcttt
  961 atcccccaaa ttactacaaa gcatagaggg ctctatgctt gctctgttcg taactcagcc
1021 actggcaagg aaagctccaa atccatgaca gtcgaagtct ctggtgcctg ccatggagac
1081 ctggcagggt ctcattcgtg actgccataa cagagacact gagaaaaaga tgcaaccatg
1141 gaaaggtgca aaggtggcaa gttctaatga catagaaaat agcaatcagc ctttctcaca
1201 tctgaaagcc ttccaaaata tctgagtgca gtagagaatt gacagaggac tgatcaccaa
1261 cctagaagta tgctcctcca ggaataggac gtcttccttt ctttactcca atagagcagc
1321 ggtgatgtca tttctgtatt tcaggaagac tggcaggaga tttatggaaa agactctgac
1381 aaggactctg gaatacaagc tcctgataac ttcaagatca taccactgga ctaagaactt
1441 tcaaaatttt aatgaacagg ctgatacctt catgaaattc aagacaaaga agaaaaaaaa
1501 ctcaatttta ttggaataaa taatcaaaag gataatgttt tcataatttt ctatttgaaa
1561 atgtgctgag tctttgaatg ttttattctc cagatttatg aacttttttc cttgaacaat
1621 tggtaaagta tacttttgta aacaaaaatt gaaacatttg cttttgctga gtgccccaga
1681 attgggaaac tattcatgag tattcatatg tttatggtaa taaagttatt tgcacaactt
1741 ca
```

An exemplary human miR1262 nucleic acid sequence is set forth below (SEQ ID NO: 172; GenBank Accession No: NR_031664.1, Version 1, incorporated herein by reference):

1 atctacaatg gtgatgggtg aatttgtaga aggatgaaag tcaaagaatc cttctgggaa
61 ctaatttttg gccttcaaca agaattgtga tat

An exemplary human RP11-360D2.1 nucleic acid sequence is set forth below (SEQ ID NO: 173; GenBank Accession No: HG492934.1, Version 1, incorporated herein by reference):

```
    1 agctgtcttc ttcgtcttat ttgttttgtc tgtgcattac ctgtggaaga aatggaagaa
  61 acaccaaaaa aagctgaaaa agcaagcctc cttagaaaaa cctggtaatg atctagaaag
121 cccattgatc aacaacattg accaaacact ccacagagtg gcaaccacag catcagtgat
181 atacaagatc tgggagcaca ggtctcacca tccttcctct aagaaaatta agcactgcaa
241 attaaagaag aagagtaaag aagaaggagc cagaagatac taaataaatg catatgcaaa
301 tgtagcttag tcaattatag atatcacaaa agaaatctat catctaagga ttaaaaattg
361 ttctttggaa acctttataa a
```

An exemplary human RP11 amino acid sequence is set forth below (SEQ ID NO: 174; GenBank Accession No: NP_056444.3, Version 3, incorporated herein by reference):

```
    1 msladellad leeaaeeeeg gsygeeeeep aiedvqeetq ldlsgdsvkt iaklwdskmf
  61 aeimmkieey iskqakasev mgpveaapey rvivdannit veienelnii hkfirdkysk
121 rfpeleslvp naldyirtvk elgnsldkck nnenlqqilt natimvvsvt asttqgqqls
181 eeelerleea cdmalelnas khriyeyves rmsfiapnls iiigastaak imgvaggltn
241 lskmpacnim llgagrktls gfsstsvlph tgyiyhsdiv qslppdlrrk aarlvaakct
301 laarvdsfhe stegkvgyel kdeierkfdk wgepppvkqv kplpapldgq rkkrggrryr
361 kmkerlglte irkganrmsf geieedayqe dlgfslghlg ksgsgrvrqt qvneatkari
421 sktlqrtlqk qsvvyggkst irdrssgtas svaftplqgl eivnpqaaek kvaeanqkyf
481 ssmaeflkvk geksglmst
```

An exemplary human RP11 nucleic acid sequence is set forth below (SEQ ID NO: 175; GenBank Accession No: NM_015629.3, Version 3, incorporated herein by reference):

```
      1 tagtttcctg tttccggctt cgcttcggcc cacccccacg tccaccccga atccctgctt
    61 aaaggccttg ctttcttgtc taacgccgca accagtcctc tgagttgcca acgtctttct
  121 tcttgtctcg acgccccgtc gtccggccac agcgattctc tgcttagcag gatcggtcca
  181 cagcgggacg tgagtccctt tcctcctcgc ggcttaccgc ctctctccgc ctagtgccag
  241 gtgctaataa agttgttgtt tcaaatgcgg ccaggaacat cgcgagcggg gaccaatcag
  301 agagtagctt tgcctctata acggcgcgag agtgagacgt catcggtgag cgactaacgc
  361 tagaaacagt ggtgcgcgga gaggagaggc ctcgggatgt ctctggcaga tgagctctta
  421 gctgatctcg aagaggcagc agaagaggag gaaggaggaa gctatgggga ggaagaagag
  481 gagccagcga tcgaggatgt gcaggaggag acacagctgg atctttccgg ggattcagtc
  541 aagaccatcg ccaagctatg ggatagtaag atgtttgctg agattatgat gaagattgag
  601 gagtatatca gcaagcaagc caaagcttca gaagtgatgg gaccagtgga ggccgcgcct
  661 gaataccgcg tcatcgtgga tgccaacaac ctgaccgtgg agatcgaaaa cgagctgaac
  721 atcatccata agttcatccg ggataagtac tcaaagagat tccctgaact ggagtccttg
  781 gtccccaatg cactggatta catccgcacg gtcaaggagc tgggcaacag cctggacaag
  841 tgcaagaaca atgagaacct gcagcagatc ctcaccaatg ccaccatcat ggtcgtcagc
  901 gtcaccgcct ccaccaccca ggggcagcag ctgtcggagg aggagctgga gcggctggag
  961 gaggcctgcg acatggcgct ggagctgaac gcctccaagc accgcatcta cgagtatgtg
1021 gagtcccgga tgtccttcat cgcacccaac ctgtccatca ttatcggggc atccacggcc
1081 gccaagatca tgggtgtggc cggcggcctg accaacctct ccaagatgcc cgcctgcaac
1141 atcatgctgc tcggggccca gcgcaagacg ctgtcgggct tctcgtctac ctcagtgctg
1201 ccccacaccg gctacatcta ccacagtgac atcgtgcagt ccctgccacc ggatctgcgg
1261 cggaaagcgg cccggctggt ggccgccaag tgcacactgg cagcccgtgt ggacagtttc
1321 cacgagagca cagaagggaa ggtgggctac gaactgaagg atgagatcga gcgcaaattc
1381 gacaagtggc aggagccgcc gcctgtgaag caggtgaagc cgctgcctgc gcccctggat
1441 ggacagcgga agaagcgagg cggccgcagg taccgcaaga tgaaggagcg gctggggctg
1501 acggagatcc ggaagcaggc caaccgtatg agcttcggag agatcgagga ggacgcctac
1561 caggaggacc tgggattcag cctgggccac ctgggcaagt cgggcagtgg gcgtgtgcgg
1621 cagacacagg taaacgaggc caccaaggcc aggatctcca agacgctgca gcggaccctg
1681 cagaagcaga gcgtcgtata tggcgggaag tccaccatcc gcgaccgctc ctcgggcacg
1741 gcctccagcg tggccttcac cccactccag ggcctggaga ttgtgaaccc acaggcggca
1801 gagaagaagg tggctgaggc caaccagaag tatttctcca gcatggctga gttcctcaag
1861 gtcaagggcg agaagagtgg ccttatgtcc acctgaatga ctgcgtgtgt ccaaggtggc
1921 ttcccactga agggacacag aggtccagtc cttctgaagg gctaggatcg ggttctggca
1981 gggagaacct gccctgccac tggccccatt gctgggactg cccagggagg aggccttgga
2041 agagtccggc ctggcctccc ccaggaccga gatcaccgcc cagtatgggc tagagcaggt
2101 cttcatcatg ccttgtcttt tttaactgag aaaggagatt ttttgaaaag agtacaatta
2161 aaaggacatt gtcaagatct gtcaaaaaaa aaaaaaaaaa a
```

An exemplary human RP1 amino acid sequence is set forth below (SEQ ID NO: 176; GenBank Accession No: AAA20120.1, Version 1, incorporated herein by reference):

```
    1 mqkwfsafdd aiiqrqwran psrggggvsf tkevdtnvat gapprrqrvp gracpwrepi
  61 rgrrgarpgg gdaggtpget vrhcsapedp ifrfsslhsy pfpgtiksrd mswkrhhlip
121 etfgvkrrrk rgpvesdplr gepgsaraav selmqlfprg lfedalppiv lrsqvyslvp
181 drtvadrqlk elgeggeiri vglgfdldah giiftedyrt rvlkacdgrp yagavqkfla
241 svlpacgdls fqqdqmtqtf gfrdseithl vnagvltvrd agswwlavpg agrfikyfvk
301 grqavlsmvr kakyrellls ellgrrapvv vrlgltyhvh dligaqlvdc isttsgtllr
361 lpet
```

An exemplary human RP1 nucleic acid sequence is set forth below (SEQ ID NO: 177; GenBank Accession No: NM_006269.1, Version 1, incorporated herein by reference):

```
      1 gacatactga gaataaatcc aaagacatta gtttctttgc acgaaatgag gttacatatc
    61 cagtgacatt tatttgagct atttaaacaa cttaaacatc tttttctttt cttaataagg
  121 gacgtttcaa gttgtggtct cagccaaaat gagtgatacc ccttctactg gtttttccat
  181 cattcatcct acgtcttctg aaggtcaagt tccaccccct cgccatttga gcctcactca
  241 tcctgttgtg gccaagcgaa tcagtttcta caagagcgga gacccccaat tcggcggggt
  301 cagggtggtg gtcaaccctc gctcctttaa gtcctttgat gctctgctgg ataacttgtc
  361 caggaaggtg cccctccctt ttggagtgag gaacatcagc acccctcggg gcaggcacag
  421 catcacgcgc ctggaggagc tggaggacgg cgagtcctac ctatgttccc acggcaggaa
  481 ggtgcagcct gtagacctgg acaaagcccg tcggcgcccg cggccctggc tcagcagccg
  541 ggccattagc gcgcactcac cgccccaccc cgtagccgtc gctgctcccg gcatgccccg
  601 ccccccacgg agcctagtgg tcttcaggaa tggcgacccg aagacgaggc gtgcggttct
  661 tctgagcagg agggtcaccc agagcttcga ggcatttcta cagcacctga cagaggtcat
  721 gcagcgccct gtggtcaagc tgtacgctac ggacggaagg agggttccca gcctccaggc
  781 agtgatcctg agctctggag ctgtggtggc ggcaggaagg gagccattta aaccaggaaa
  841 ttatgacatc caaaaatact tgcttcctgc tagattacca gggatctctc agcgtgtgta
  901 ccccaaggga aatgcaaagt cagaaagcag aaagataagc acacatatgt cttcaagctc
  961 aaggtcccag atttattctg tttcttctga gaaaacacat aataatgatt gctacttaga
1021 ctattctttt gttcctgaaa agtacttggc cttagaaaag aatgattctc agaatttacc
1081 aatatatcct tctgaagatg atattgagaa atcaattatt tttaatcaag acggcactat
1141 gacagttgag atgaaagttc gattcagaat aaaagaggaa gaaaccataa aatggacaac
1201 tactgtcagt aaaactggtc cttctaataa tgatgaaaag agtgagatga gttttccagg
1261 aagaacagaa agtcgatcat ctggtttaaa gcttgcagca tgttcattct ctgcagatgt
1321 gtcacctatg gagcgaagca gtaatcaaga gggcagtttg gcagaggaga taaacattca
1381 aatgacagat caagtggctg aaacttgcag ttctgctagt tgggagaatg ctactgtgga
1441 cacagatatc atccagggaa ctcaagacca agcaaagcat cgtttttata ggccccctac
1501 acctggacta agaagagtga gacaaaagaa atctgtgatt ggcagtgtga ccttagtatc
1561 tgaaactgag gttcaagaga aaatgattgg acagttttca tatagtgaag aaagggaaag
1621 tggggaaaac aagtctgagt atcacatgtt tacacattct tgcagtaaaa tgtcatcagt
1681 atctaacaaa ccagtacttg ttcagatcaa taacaatgat caaatggagg agtcatcatt
1741 agaaagaaaa aaggaaaaca gtctgcttaa gtcaagtgca ataagtgctg gtgttataga
1801 aattacaagt cagaagatgt tagagatgtc acataataat ggtttgccat caactatatc
1861 aaataactca attgtggagg aagatgtagt tgattgtgtg gtattggaca acaaaactgg
1921 tatcaagaac ttcaaaactt atggtaacac caatgatagg ttcagtccta tttcagcaga
1981 tgcaacccat ttttcaagta ataactctgg aactgacaaa aatatttctg aggctccagc
2041 ttcagaagca tcctctactg tcactgcaag aattgacaga ctaattaatg aatttgctca
2101 gtgtggttta acaaaacttc caaaaaatga aaagaagatt ttgtcatctg ttgccagcaa
2161 aaagaagaaa aaatctcgac agcaagcaat aaattccagg tatcaagatg gacagcttgc
2221 aaccaaagga attcttaata agaatgagag aataaacaca aaaggtagaa ttacaaagga
2281 aatgatagtg caagattcag atagtcccct taaaggaggg atactttgtg aggaagacct
2341 ccagaaaagt gatactgtaa ttgaatcaaa tactttttgt tccaaaagta atctcaattc
2401 cacgatttcc aagaatttcc atagaaataa attaaatact actcaaaatt ccaaggttca
2461 aggactttta accaaaagaa aatctagatc actaaataaa ataagcttag gagcacctaa
2521 aaaaagagaa atcggtcaaa gagataaagt gtttcctcac aatgaatcta aatattgcaa
2581 aagtactttt gaaaacaaaa gtttatttca tgtatttaac atccttgagc aaaaacccaa
2641 agatttttat gcaccgcaat ctcaagcaga agtggcatct gggtatttga gaggaatggc
2701 aaagaagagt ttagtttcaa aagttactga ttcacacata actttaaaaa gccagaaaaa
2761 acgtaaaggg gataaagtga aagcaagtgc tattttaagt aaacaacatg ctacaaccag
2821 ggcaaattct ttagcttctt tgaaaaaacc tgattttcct gaggctattg ctcatcattc
2881 aattcaaaat tatatacaga gttggttgca gaacataaat ccatatccaa ctttaaagcc
2941 tataaaatca gctccagtat gtagaaatga aacgagtgtg gtaaattgta gcaataatag
3001 tttttcaggg aatgatcccc atacaaattc tggaaaaata agtaattttg ttatggaaag
3061 taataagcac ataactaaaa ttgccggttt gacaggagat aatctatgta aagagggaga
3121 taagtctttt attgccaatg acactggtga agaagatctc catgagacac aggttggatc
3181 tctgaatgat gcttatttgg ttcccctgca tgaacactgt actttgtcac agtcagctat
3241 taatgatcat aatactaaaa gtcatatagc tgctgaaaaa tcaggaccag agaaaaaact
3301 tgtttaccag gaaataaacc tagctagaaa aaggcaaagt gtagaggctg ccattcaagt
3361 agatcctata gaagaggaaa ctccaaaaga cctcttacca gtcctgatgc ttcaccaatt
3421 gcaagcttca gttcctggta ttcacaagac tcagaatgga gttgttcaaa tgccaggttc
3481 acttgcaggt gttccctttc attctgcaat atgtaattca tccactaatc tccttctagc
3541 ttggctcttg gtgctaaacc taaagggaag tatgaatagc ttctgtcaag ttgatgctca
3601 caaggctacc aacaaatctt cagaaacact tgcattgttg gagattctaa agcacatagc
3661 tatcacagag gaagctgatg acttgaaagc tgctgttgcc aatttagtgg agtcaactac
3721 aagccacttt ggactcagtg agaaagaaca agacatggtt ccaatagatc tttctgcaaa
3781 ttgttccacg gtcaacattc agagtgttcc taagtgcagt gaaaatgaaa gaacacaagg
3841 aatctcctct ttggatggag gttgctctgc cagtgaggca tgtgcccctg aagtctgtgt
3901 tttggaagtg acttgctctc catgtgagat gtgcactgta aataaggctt attctccaaa
3961 agagacatgt aaccccagtg acactttttt tcctagtgat ggttatggtg tggatcagac
4021 ttctatgaat aaggcttgtt tcctaggaga ggtctgttca cttactgata ctgtgttttc
4081 tgataaggct tgtgctcaaa aggagaacca tacctatgag ggagcttgcc caattgatga
4141 gacctacgtt cctgtcaatg tctgcaatac cattgacttt ttaaactcca aagaaaacac
4201 atatactgat aacttggatt caactgaaga gttagaaaga ggtgatgaca ttcagaaaga
4261 tctaaatatt ttgacagacc ctgaatataa aaatggattt aatacattgg tgtcacatca
4321 aaatgtcagt aatttaagct cctgtggcct ttgcctaagt gaaaaagaag cagaacttga
4381 taagaaacat agttctctag atgattttga aaattgttca ctaaggaagt ttcaggatga
4441 aaatgcatat acttcctttg atatggaaga accacggact tctgaagaac caggctcaat
4501 aaccaacagc atgacatcaa gtgaaagaaa catttcagaa ttggaatctt ttgaagaatt
4561 agaaaaccat gacactgata tctttaatac agtggtaaat ggaggagagc aagccactga
4621 agaattaatc caagaagagg tagaggctag taaaacttta gaattgatag acatctctag
4681 taagaatatt atggaagaaa aaagaatgaa cggtataatt tatgaaataa tcagtaagag
4741 gctggcaaca ccaccatctt tagatttttg ctatgattct aagcaaaata gtgaaaagga
4801 gaccaatgaa ggagaaacta agatggtaaa aatgatggtg aaaactatgg aaactggaag
4861 ttattcagag tcctctcctg atttaaaaaa atgcatcaaa agtccagtga cttctgattg
4921 gtcagactat cggcctgaca gtgacagtga gcagccatat aaaacatcca gtgatgatcc
4981 caatgacagt ggcgaactta cccaagagaa agaatataac ataggatttg ttaaaagggc
5041 aatagaaaaa ctgtacggta aagcagatat tatcaaacca tctttttttc ctgggtctac
5101 ccgcaaatct caggtttgtc cttataattc tgtggaattt cagtgttcca ggaaagcaag
5161 tctttatgat tctgaagggc agtcatttgg ctcttctgaa caggtatcta gtagttcatc
5221 tatgttgcag gaattccagg aggaaagaca agataagtgt gatgttagtg ctgtgaggga
5281 caattattgt aggggtgaca ttgtagaacc tggtacaaaa caaaatgatg atagcagaat
5341 cctcacagac atagaggaag gagtactgat tgacaaaggc aaatggcttc tgaaagaaaa
5401 tcatttgcta aggatgtcat ctgaaaatcc tggcatgtgt ggcaatgcag acaccacatc
5461 agtggacacc ctacttgata ataacagcag tgaggtacca tattcacatt ttggtaattt
5521 ggccccaggc ccaacgatgg atgaactctc ctcttcagaa ctcgaggaac tgactcaacc
5581 ccttgaacta aaatgcaatt actttaacat gcctcatggt agtgactcag aaccttttca
5641 tgaggacttg ctggatgttc gcaatgaaac ctgtgccaag gaaagaatag caaatcatca
5701 tacagaggag aagggtagtc atcagtcaga aagagtatgc acatctgtca ctcattcctt
5761 tatttctgct ggtaacaaag tctaccctgt ctctgatgat gctattaaaa accaaccatt
5821 gcctggcagt aatatgattc atggtacact tcaggaagct gactctttgg ataaactgta
5881 tgctctttgt ggtcaacatt gcccaatact aactgttatt atccaaccca tgaatgagga
5941 agaccgagga tttgcatatc gcaaagaatc tgatattgaa aatttcttgg gtttttattt
6001 atggatgaaa atacacccat atttacttca gacagacaaa aatgtgttca gggaagagaa
6061 caataaagca agtatgagac aaaatcttat tgataatgcc attggtgata tatttgatca
6121 gttttatttc agtaacacat ttgacttgat gggtaaaaga agaaaacaaa aaagaattaa
6181 cttcttgggg ttagaggaag aaggtaattt aaagaaattt caaccagatt tgaaggaaag
6241 gttttgtatg aatttcttgc acacatcatt gttagttgtg ggtaatgtgg attcaaatac
6301 acaagacctc agcggtcaga caaatgaaat ctttaaagca gtcgatgaga ataacaactt
6361 attaaataac agattccagg gctcaagaac aaatctcaac caagtagtaa gagaaaatat
6421 caactgtcat tacttctttg aaatgcttgg tcaagcttgc ctcttagata tttgccaagt
6481 tgagacctcc ttaaatatta gcaacagaaa tattttagaa ctttgtatgt ttgagggtga
6541 aaatcttttc atttgggaag aggaagacat attaaattta actgatcttg aaagcagtag
6601 agaacaagaa gatttataat ttcaatatca gcacactcat tctttgtcaa ttcatttttt
6661 cccatgagat gaagcacatg tgacgaatac ggactagata acctctaaga attttccact
6721 tcttcaaaat gaacttactc tagaaagctt acccttggat aaccagtttg actttcataa
6781 tgtctctgtt ttttgttttt ccaacaatta cagactcagg ttctcttatt ttggaagttt
6841 ctatctggtt ttgttctgaa cttacatttt tttttttttt ggtatctatg attttttttg
6901 ctcagggcat caaaatgtgc taaggacaag aattatatcc tttttaaaaa atgttgttag
6961 cttggtgtaa aatgtatatt gactgtattg gtgaataaat tgaatagaca taacctcaaa
7021 gtacttcact tattcttttt aactactgat ttgataaaaa gtatgattat aagatatcca
7081 cgacaatctc atagtttctt
```

An exemplary human CD28 amino acid sequence is set forth below (SEQ ID NO: 178; GenBank Accession No: AAI12086.1, Version 1, incorporated herein by reference):

| | |
|---|---|
| 1 | mlrlllalnl fpsiqvtgnk ilvkqspmlv aydnavnlsc |
| | kysynlfsre fraslhkgld |
| 61 | savevcvvyg nysqqlqvys ktgfncdgkl gnesvtfylq |
| | nlyvnqtdiy fckievmypp |
| 121 | pyldneksng tiihvkgkhl cpsplfpgps kpfwvlvvvg |
| | gvlacysllv tvafiifwvr |
| 181 | skrsrllhsd ymnmtprrpg ptrkhyqpya pprdfaayrs |

An exemplary human CD28 nucleic acid sequence is set forth below (SEQ ID NO: 179; GenBank Accession No: AJ295273.1, Version 1, incorporated herein by reference):

| | |
|---|---|
| 1 | atgctcaggc tgctcttggc tctcaactta ttcccttcaa |
| | ttcaagtaac aggaaacaag |
| 61 | attttggtga agcagtcgcc catgcttgta gcgtacgaca |
| | atgcggtcaa ccttagctac |
| 121 | aatgagaaga gcaatggaac cattatccat gtgaaaggtg |
| | aggagtaaga ggagcaggct |
| 181 | cctgcacagt gactacatga acatgactcc ccgccgcccc |
| | gggcccaccc gcaagcatta |
| 241 | ccagccctat gccccaccac gcgacttcgc agcctatcgc |
| | tcctga |

An exemplary human ICOS amino acid sequence is set forth below (SEQ ID NO: 180; GenBank Accession No: AAH28006.1, Version 1, incorporated herein by reference):

| | |
|---|---|
| 1 | mksglwyffl fclrikvltg eingsanyem fifhnggvqi |
| | lckypdivqq fkmqllkggq |
| 61 | ilcdltktkg sgntvsiksl kfchsqlsnn sysfflynld |
| | hshanyyfcn lsifdpppfk |
| 121 | vtltggylhi yesqlccqlk fwlpigcaaf vvvcilgcil |
| | icwltkkm |

An exemplary human ICOS nucleic acid sequence is set forth below (SEQ ID NO: 181; GenBank Accession No: NM_012092.3, Version 3, incorporated herein by reference):

| | |
|---|---|
| 1 | cgagagcctg aattcactgt cagctttgaa cactgaacgc |
| | gaggactgtt aactgtttct |
| 61 | ggcaaacatg aagtcaggcc tctggtattt ctttctcttc |
| | tgcttgcgca ttaaagtttt |
| 121 | aacaggagaa atcaatggtt ctgccaatta tgagatgttt |
| | atatttcaca acggaggtgt |
| 181 | acaaatttta tgcaaatatc ctgacattgt ccagcaattt |
| | aaaatgcagt tgctgaaagg |
| 241 | ggggcaaata ctctgcgatc tcactaagac aaaaggaagt |
| | ggaaacacag tgtccattaa |
| 301 | gagtctgaaa ttctgccatt ctcagttatc caacaacagt |
| | gtctcttttt ttctatacaa |
| 361 | cttggaccat tctcatgcca actattactt ctgcaaccta |
| | tcaatttttg atcctcctcc |
| 421 | ttttaaagta actcttacag gaggatattt gcatatttat |
| | gaatcacaac tttgttgcca |
| 481 | gctgaagttc tggttaccca taggatgtgc agcctttgtt |
| | gtagtctgca ttttgggatg |
| 541 | catacttatt tgttggctta caaaaaagaa gtattcatcc |
| | agtgtgcacg accctaacgg |
| 601 | tgaatacatg ttcatgagag cagtgaacac agccaaaaaa |
| | tctagactca cagatgtgac |
| 661 | cctataatat ggaactctgg cacccaggca tgaagcacgt |
| | tggccagttt tcctcaactt |
| 721 | gaagtgcaag attctcttat ttccgggacc acggagagtc |
| | tgacttaact acatacatct |
| 781 | tctgctggtg ttttgttcaa tctggaagaa tgactgtatc |
| | agtcaatggg gattttaaca |
| 841 | gactgccttg gtactgccga gtcctctcaa aacaaacacc |
| | ctcttgcaac cagctttgga |
| 901 | gaaagcccag ctcctgtgtg ctcactggga gtggaatccc |
| | tgtctccaca tctgctccta |
| 961 | gcagtgcatc agccagtaaa acaaacacat ttacaagaaa |
| | aatgttttaa agatgccagg |
| 1021 | ggtactgaat ctgcaaagca aatgagcagc caaggaccag |
| | catctgtccg catttcacta |
| 1081 | tcatactacc tcttctttct gtagggatga gaattcctct |
| | tttaatcagt caagggagat |
| 1141 | gcttcaaagc tggagctatt ttatttctga gatgttgatg |
| | tgaactgtac attagtacat |
| 1201 | actcagtact ctccttcaat tgctgaaccc cagttgacca |
| | ttttaccaag actttagatg |
| 1261 | ctttcttgtg ccctcaattt tctttttaaa aatacttcta |
| | catgactgct tgacagccca |
| 1321 | acagccactc tcaatagaga gctatgtctt acattctttc |
| | ctctgctgct caatagtttt |
| 1381 | atatatctat gcatacatat atacacacat atgtatataa |
| | aattcataat gaatatattt |
| 1441 | gcctatattc tccctacaag aatatttttg ctccagaaag |
| | acatgttctt ttctcaaatt |
| 1501 | cagttaaaat ggtttacttt gttcaagtta gtggtaggaa |
| | acattgcccg gaattgaaag |
| 1561 | caaatttatt ttattatcct attttctacc attatctatg |
| | ttttcatggt gctattaatt |
| 1621 | acaagtttag ttctttttgt agatcatatt aaaattgcaa |
| | acaaaatcat ctttaatggg |
| 1681 | ccagcattct catggggtag agcagaatat tcatttagcc |
| | tgaaagctgc agttactata |
| 1741 | ggttgctgtc agactatacc catggtgcct ctgggcttga |
| | caggtcaaaa tggtccccat |
| 1801 | cagcctggag cagccctcca gacctgggtg gaattccagg |
| | gttgagagac tcccctgagc |
| 1861 | cagaggccac taggtattct tgctcccaga ggctgaagtc |
| | accctgggaa tcacagtggt |
| 1921 | ctacctgcat tcataattcc aggatctgtg aagagcacat |
| | atgtgtcagg gcacaattcc |
| 1981 | ctctcataaa aaccacacag cctggaaatt ggccctggcc |
| | cttcaagata gccttcttta |
| 2041 | gaatatgatt tggctagaaa gattcttaaa tatgtggaat |
| | atgattattc ttagctggaa |
| 2101 | tattttctct acttcctgtc tgcatgccca aggcttctga |
| | agcagccaat gtcgatgcaa |
| 2161 | caacatttgt aactttaggt aaactgggat tatgttgtag |
| | tttaacattt tgtaactgtg |
| 2221 | tgcttatagt ttacaagtga gacccgatat gtcattatgc |
| | atacttatat tatcttaagc |
| 2281 | atgtgtaatg ctggatgtgt acagtacagt actgaacttg |
| | taatttgaat ctagtatggt |
| 2341 | gttctgtttt cagctgactt ggacaacctg actggctttg |
| | cacaggtgtt ccctgagttg |
| 2401 | tttgcaggtt tctgtgtgtg gggtggggta tggggaggag |
| | aaccttcatg gtggcccacc |
| 2461 | tggcctggtt gtccaagctg tgcctcgaca catcctcatc |
| | cccagcatgg gacacctcaa |
| 2521 | gatgaataat aattcacaaa atttctgtga aatcaaatcc |
| | agttttaaga ggagccactt |
| 2581 | atcaaagaga ttttaacagt agtaagaagg caaagaataa |
| | acatttgata ttcagcaact |
| 2641 | gaaaaaaaaa aa |

An exemplary human EOMES amino acid sequence is set forth below (SEQ ID NO: 182; GenBank Accession No: NP_001265111.1, Version 1, incorporated herein by reference):

| | |
|---|---|
| 1 | mqlgeqllvs svnlpgahfy plesarggsg gsaghlpsaa |
| | pspqkldldk askkfsgsls |
| 61 | ceavsgepaa asagapaaml sdtdagdafa saaavakpgp |
| | pdgrkgspcg eeelpsaaaa |
| 121 | aaaaaaaaaa tarysmdsls seryylqspg pqgselaapc |
| | slfpyqaaag aphgpvypap |
| 181 | ngarypygsm lppggfpaav cppgraqfgp gagagsgagg |
| | ssgggggpgt yqysqgaply |
| 241 | gpypgaaaag scgglgglgv pgsgfrahvy lcnrplwlkf |
| | hrhqtemiit kqgrrmfpfl |
| 301 | sfninglnpt ahynvfvevv ladpnhwrfq ggkwvtcgka |
| | dnnmqgnkmy vhpespntgs |
| 361 | hwmrqeisfg klkltnnkga nnnntqmivl qslhkyqprl |
| | hivevtedgv edlnepsktq |
| 421 | tftfsetqfi avtayqntdi tqlkidhnpf akgfrdnyds |
| | mytasendrl tpsptdsprs |
| 481 | hqivpggryg vqsffpepfv ntlpqaryyn gertvpqtng |
| | llspqqseev anppqrwlvt |
| 541 | pvqqpgtnkl dissyeseyt sstllpygik slplqtshal |
| | gyypdptfpa magwggrgsy |
| 601 | qrkmaaglpw tsrtsptvfs edqlskekvk eeigsswiet |
| | ppsiksldsn dsgvytsack |
| 661 | rrrlspsnss nenspsikce dinaeeyskd tskgmggyya |
| | fyttp |

An exemplary human EOMES nucleic acid sequence is set forth below (SEQ ID NO: 183; GenBank Accession No: NM_001278182.1, Version 1, incorporated herein by reference):

| | |
|---|---|
| 1 | aagtttccaa gtggtcaact tgaccgatgc tttggcaatt |
| | gaaaaagggc agaaaggcgc |
| 61 | gggctagtgg gtggatgggg acaaagatct aagtcacctt |
| | cttccagcgt gtgagcctgg |
| 121 | gaggagggtg ggggtcctga ggagcaagag gtacgaggaa |
| | ggaaaaggag agggcttctg |
| 181 | ggttagtttc cacctcctgc tttccaactc acggcgcttt |
| | ccttccggaa aggacgctgg |
| 241 | attcagggcg cgccagtacg cgcagtagcg gcccgcgagt |
| | cggcaggtgg gtagccccgg |
| 301 | cgcgggagga aggggaagtt accttcccct cggaagaggg |
| | cgctggctcc cccatcctgc |
| 361 | ctttataata aggccaccgg aggagaggaa gcagccagct |
| | gccgtctgcg ctttgcaaag |
| 421 | catgcagtta ggggagcagc tcttggtgag ctcagtgaac |
| | ctgcctggcg cgcacttcta |
| 481 | cccgctggag agtgcgcgag gcggcagcgg cgggagcgct |
| | ggccacctcc ccagcgcggc |
| 541 | cccctctcct cagaagttgg acttagacaa agcgtccaag |
| | aagttttccg gcagtctctc |
| 601 | ctgcgaggcg gtgagcgggg agcccgcagc cgccagcgca |
| | ggggcccccg cggccatgct |
| 661 | tagtgacacc gacgccgggg acgcatttgc cagcgctgcg |
| | gcagtggcca agccggggcc |
| 721 | cccggacggc cgcaagggct ccccctgcgg ggaggaggag |
| | ctgccctccg ccgctgcagc |
| 781 | cgccgccgcc gccgccgccg cggctgcggc cactgcgcgc |
| | tactccatgg acagcctgag |
| 841 | ctccgagcgg tactacctcc agtcccccgg tcctcagggg |
| | tcggagctgg ctgcgccctg |
| 901 | ctcactcttc ccgtaccagg cggcggctgg ggcgccccac |
| | ggacctgtgt acccggctcc |
| 961 | taacggggcg cgctacccct acggctccat gctgcccccc |
| | ggcggcttcc ccgcggctgt |
| 1021 | gtgcccaccc gggagggcgc agttcggccc aggagccggt |
| | gcgggcagtg gcgcgggcgg |
| 1081 | tagcagcggc gggggcggcg gcccgggcac ctatcagtac |
| | agccaggggg ctccgctcta |
| 1141 | cgggccgtac cctggagccg cagcggcggg atcttgcgga |
| | ggactggggg gcctgggggt |
| 1201 | tccaggttct ggcttccgtg cccacgtcta cctgtgcaac |
| | cggcctctgt ggctcaaatt |
| 1261 | ccaccgccac caaactgaga tgatcattac gaaacagggc |
| | aggcgcatgt ttcctttctt |
| 1321 | gagcttcaac ataaacggac tcaatcccac tgcccactac |
| | aatgtgttcg tagaggtggt |
| 1381 | gctggcggac cccaaccact ggcgcttcca ggggggcaaa |
| | tgggtgacct gtggcaaagc |
| 1441 | cgacaataac atgcagggca acaaaatgta tgttcaccca |
| | gagtctccta atactggttc |
| 1501 | ccactggatg agacaggaga tttcattcgg gaaattaaaa |
| | ctcaccaata acaaaggcgc |
| 1561 | aaataacaac aacacccaga tgatagtctt acaatcctta |
| | cacaaatacc aaccccgact |
| 1621 | gcatattgtt gaagttacag aggatggcgt ggaggacttg |
| | aatgagccct caaagaccca |
| 1681 | gacttttacc ttctcagaaa cgcaattcat tgcagtgact |
| | gcctaccaaa acaccgatat |
| 1741 | tactcaacta aagattgatc ataacccctt tgcaaaaggc |
| | ttcagagaca actatgattc |
| 1801 | catgtacacc gcttcagaaa atgacaggtt aactccatct |
| | cccacggatt ctcctagatc |
| 1861 | ccatcagatt gtccctggag gtcggtacgg cgttcaatcc |
| | ttcttcccgg agccctttgt |
| 1921 | caacacttta cctcaagccc gctattataa tggcgagaga |
| | accgtgccac agaccaacgg |
| 1981 | cctcctttca ccccaacaga gcgaagaggt ggccaaccct |
| | ccccagcggt ggcttgtcac |
| 2041 | gcctgtccag caacctggga ccaacaaact agacatcagt |
| | tcctatgaat ctgaatatac |
| 2101 | ttctagcaca ttgctcccat atggcattaa atccttgccc |
| | cttcagacat cccatgccct |
| 2161 | ggggtattac ccagacccaa cctttcctgc aatggcaggg |
| | tggggaggtc gaggttctta |
| 2221 | ccagaggaag atggcagctg gactaccatg gacctccaga |
| | acaagcccca ctgtgttctc |
| 2281 | tgaagatcag ctctccaagg agaaagtgaa agaggaaatt |
| | ggctcttctt ggatagagac |
| 2341 | acccccttcc atcaaatctc tagattccaa tgattcagga |
| | gtatacacca gtgcttgtaa |
| 2401 | gcgaaggcgg ctgtctccta gcaactccag taatgaaaat |
| | tcaccctcca taaagtgtga |
| 2461 | ggacattaat gctgaagagt atagtaaaga cacctcaaaa |
| | ggcatgggag ggtattatgc |
| 2521 | tttttacaca actccctaaa gagttatttt aacctcaaaa |
| | attagctaac tttttgcaga |
| 2581 | tggacttggt ggtgtttttt gttgtcttct ttgcctaggt |
| | tgccaaaaag atgtttgcct |
| 2641 | tccaccttga tgcatcctgt tttgtgcaat tctctaaaag |
| | aaggtgccaa agctttttga |
| 2701 | ttgctgcagg taactgaaac aaacctagca tttttaaaaa |
| | ataagattaa tggaagactt |
| 2761 | taaggtattt taaaattcga agggtatcca aggttctgta |
| | tttatttatt ggggagacac |
| 2821 | taacccttca aagaagcagg ctgtgaacat tgggtgccca |
| | gtgctatcag atgagttaaa |
| 2881 | acctttgatt ctcatttcta tttgtaaatt cttaagcaaa |
| | tagaagccga gtgttaaggt |
| 2941 | gttttgcttc tgaaagaggg ctgtgccttc cgtttcagaa |
| | ggagacattt tgctgttaca |
| 3001 | ttctgccagg ggcaaaagat actaggccca ggagtcaaga |
| | aaagcttttg tgaaagtgat |
| 3061 | agtttcacct gactttgatt ccttaacccc cggcttttgg |
| | aacaagccat gtttgcccta |
| 3121 | gtccaggatt gcctcacttg agacttgcta ggcctctgct |
| | gtgtgctggg gtggccagtg |
| 3181 | ggactcagga gagagcaagc taaggagtca ccaaaaaaaa |
| | aaaaaaaaaa aagggagaat |
| 3241 | ttaaaagtgt acagttgtgt gtttagatac actatagaat |
| | aatgtggtat atattgtaca |
| 3301 | aatagtctac ataggtgtct gggataatgt aaaactggtg |
| | ctttggcttt gtaaagaatt |
| 3361 | tgcaaatcac ttaacagctg caggggcaag gggagagttt |
| | catcatcccc atgatatttg |
| 3421 | ggaatattct gtttacttct tagatagtta agaatgtatt |
| | cagctactat gtactaactt |
| 3481 | gaaccgtgtt taaggaaaac tcctatttca tcctcttctt |
| | gcgccatccc ctctccctaa |
| 3541 | cttggtaatg tgaagaaact aaaacctgat accacagctc |
| | ctataggcat tttagagatc |
| 3601 | ttggattttt atgtacagtc ttagtcattt ttaataaatg |
| | tggttcagta agggaacgga |
| 3661 | aaaaaaaaaa aaaa |

An exemplary human IL2RB amino acid sequence is set forth below (SEQ ID NO: 184; GenBank Accession No: CAG30392.1, Version 1, incorporated herein by reference):

| | |
|---|---|
| 1 | maapalswrl pllilllpla tswasaavng tsqftcfyns |
| | raniscvwsq dgalqdtscq |
| 61 | vhawpdrrrw nqtcellpvs qaswacnlil gapdsqkltt |
| | vdivtlrvlc regvrwrvma |
| 121 | iqdfkpfenl rlmapislqv vhvethrcni sweisqashy |
| | ferhlefear tlspghtwee |
| 181 | aplltlkqkq ewicletltp dtqyefqvrv kplqgefttw |
| | spwsqplafr tkpaalgkdt |
| 241 | ipwlghllvg lsgafgfiil vyllincrnt gpwlkkvlkc |
| | ntpdpskffs qlssehggdv |
| 301 | qkwlsspfps ssfspgglap eisplevler dkvtqlllqq |
| | dkvpepasls snhsltscft |
| 361 | nqgyfffhlp daleieacqv yftydpysee dpdegvagap |
| | tgsspqplqp lsgeddayct |
| 421 | fpsrddlllf spsllggpsp pstapggsga geermppslq |
| | ervprdwdpq plgpptpgvp |
| 481 | dlvdfqpppe lvlreageev pdagpregvs fpwsrppgqg |
| | efralnarlp lntdaylslq |
| 541 | elqgqdpthl v |

An exemplary human IL2RB nucleic acid sequence is set forth below (SEQ ID NO: 185; GenBank Accession No: NM_000878.3, Version 3, incorporated herein by reference):

| | |
|---|---|
| 1 | gcagccagag ctcagcaggg ccctggagag atggccacgg |
| | tcccagcacc ggggaggact |
| 61 | ggagagcgcg cgctgccacc gccccatgtc tcagccaggg |
| | cttccttcct cggctccacc |
| 121 | ctgtggatgt aatggcggcc cctgctctgt cctggcgtct |
| | gcccctcctc atcctcctcc |
| 181 | tgcccctggc tacctcttgg gcatctgcag cggtgaatgg |
| | cacttcccag ttcacatgct |
| 241 | tctacaactc gagagccaac atctcctgtg tctggagcca |
| | agatggggct ctgcaggaca |
| 301 | cttcctgcca agtccatgcc tggccggaca gacggcggtg |
| | gaaccaaacc tgtgagctgc |
| 361 | tccccgtgag tcaagcatcc tgggcctgca acctgatcct |
| | cggagcccca gattctcaga |
| 421 | aactgaccac agttgacatc gtcaccctga gggtgctgtg |
| | ccgtgagggg gtgcgatgga |
| 481 | gggtgatggc catccaggac ttcaagccct ttgagaacct |
| | tcgcctgatg gcccccatct |
| 541 | ccctccaagt tgtccacgtg gagacccaca gatgcaacat |
| | aagctgggaa atctcccaag |
| 601 | cctcccacta ctttgaaaga cacctggagt tcgaggcccg |
| | gacgctgtcc ccaggccaca |
| 661 | cctgggagga ggcccccctg ctgactctca agcagaagca |
| | ggaatggatc tgcctggaga |
| 721 | cgctcacccc agacacccag tatgagtttc aggtgcgggt |
| | caagcctctg caaggcgagt |
| 781 | tcacgacctg gagcccctgg agccagcccc tggccttcag |
| | gacaaagcct gcagcccttg |
| 841 | ggaaggacac cattccgtgg ctcggccacc tcctcgtggg |
| | cctcagcggg gcttttggct |
| 901 | tcatcatctt agtgtacttg ctgatcaact gcaggaacac |
| | cgggccatgg ctgaagaagg |
| 961 | tcctgaagtg taacacccca gacccctcga agttcttttc |
| | ccagctgagc tcagagcatg |
| 1021 | gaggagacgt ccagaagtgg ctctcttcgc ccttcccctc |
| | atcgtccttc agccctggcg |
| 1081 | gcctggcacc tgagatctcg ccactagaag tgctggagag |
| | ggacaaggtg acgcagctgc |
| 1141 | tcctgcagca ggacaaggtg cctgagcccg catccttaag |
| | cagcaaccac tcgctgacca |
| 1201 | gctgcttcac caaccagggt tacttcttct tccacctccc |
| | ggatgccttg gagatagagg |
| 1261 | cctgccaggt gtactttact tacgacccct actcagagga |
| | agaccctgat gagggtgtgg |
| 1321 | ccggggcacc cacagggtct tccccccaac ccctgcagcc |
| | tctgtcaggg gaggacgacg |
| 1381 | cctactgcac cttcccctcc agggatgacc tgctgctctt |
| | ctcccccagt ctcctcggtg |
| 1441 | gccccagccc cccaagcact gcccctgggg gcagtggggc |
| | cggtgaagag aggatgcccc |
| 1501 | cttctttgca agaaagagtc cccagagact gggaccccca |
| | gcccctgggg cctcccaccc |
| 1561 | caggagtccc agacctggtg gattttcagc caccccctga |
| | gctggtgctg cgagaggctg |
| 1621 | gggaggaggt ccctgacgct ggccccaggg agggagtcag |
| | tttcccctgg tccaggcctc |
| 1681 | ctgggcaggg ggagttcagg gcccttaatg ctcgcctgcc |
| | cctgaacact gatgcctact |
| 1741 | tgtccctcca agaactccag ggtcaggacc caactcactt |
| | ggtgtagaca gatggccagg |
| 1801 | gtgggaggca ggcagctgcc tgctctgcgc cgagcctcag |
| | aaggaccctg ttgagggtcc |
| 1861 | tcagtccact gctgaggaca ctcagtgtcc agttgcagct |
| | ggacttctcc acccggatgg |
| 1921 | cccccaccca gtcctgcaca cttggtccat ccatttccaa |
| | acctccactg ctgctcccgg |
| 1981 | gtcctgctgc ccgagccagg aactgtgtgt gttgcagggg |
| | ggcagtaact ccccaactcc |
| 2041 | ctcgttaatc acaggatccc acgaatttag gctcagaagc |
| | atcgctcctc tccagccctg |
| 2101 | cagctattca ccaatatcag tcctcgcggc tctccagggc |
| | tccctgccct gacctcttcc |
| 2161 | ctgggttttc tgccccagcc tcctccttcc ctcccctccc |
| | cgtccacagg gcagcctgag |
| 2221 | cgtgctttcc aaaacccaaa tatggccacg ctccccctcg |
| | gttcaaaacc ttgcacaggt |
| 2281 | cccactgccc tcagccccac ttctcagcct ggtacttgta |
| | cctccggtgt cgtgtgggga |
| 2341 | catccccttc tgcaatcctc cctaccgtcc tcctgagcca |
| | ctcagagctc cctcacaccc |
| 2401 | cctctgttgc acatgctatt ccctggggct gctgtgcgct |
| | ccccctcatc taggtgacaa |
| 2461 | acttccctga ctcttcaagt gccggttttg cttctcctgg |
| | agggaagcac tgcctccctt |
| 2521 | aatctgccag aaacttctag cgtcagtgct ggagggagaa |
| | gctgtcaggg acccagggcg |
| 2581 | cctggagaaa gaggccctgt tactattcct ttgggatctc |
| | tgaggcctca gagtgcttgg |
| 2641 | ctgctgtatc tttaatgctg gggcccaagt aagggcacag |
| | atccccccac aaagtggatg |
| 2701 | cctgctgcat cttcccacag tggcttcaca gacccacaag |
| | agaagctgat ggggagtaaa |
| 2761 | ccctggagtc cgaggcccag gcagcagccc cgcctagtgg |
| | tgggccctga tgctgccagg |
| 2821 | cctgggacct cccactgccc cctccactgg aggggtctcc |
| | tctgcagctc agggactggc |
| 2881 | acactggcct ccagaagggc agctccacag ggcagggcct |
| | cattattttt cactgcccca |
| 2941 | gacacagtgc ccaacacccc gtcgtatacc ctggatgaac |
| | gaattaatta cctggcacca |
| 3001 | cctcgtctgg gctccctgcg cctgacattc acacagagag |
| | gcagagtccc gtgcccatta |
| 3061 | ggtctggcat gccccctcct gcaaggggct caacccccta |
| | ccccgacccc tccacgtatc |
| 3121 | tttcctaggc agatcacgtt gcaatggctc aaacaacatt |
| | ccaccccagc aggacagtga |
| 3181 | ccccagtccc agctaactct gacctgggag ccctcaggca |
| | cctgcactta caggccttgc |
| 3241 | tcacagctga ttgggcacct gaccacacgc ccccacaggc |
| | tctgaccagc agcctatgag |
| 3301 | ggggtttggc accaagctct gtccaatcag gtaggctggg |
| | cctgaactag ccaatcagat |
| 3361 | caactctgtc ttgggcgttt gaactcaggg agggaggccc |
| | ttgggagcag gtgcttgtgg |
| 3421 | acaaggctcc acaagcgttg agccttggaa aggtagacaa |
| | gcgttgagcc actaagcaga |
| 3481 | ggaccttggg ttcccaatac aaaaatacct actgctgaga |
| | gggctgctga ccatttggtc |
| 3541 | aggattcctg ttgcctttat atccaaaata aactcccctt |
| | tcttgaggtt gtctgagtct |
| 3601 | tgggtctatg ccttgaaaaa agctgaatta ttggacagtc |
| | tcacctcctg ccatagggtc |
| 3661 | ctgaatgttt cagaccacaa ggggctccac acctttgctg |
| | tgtgttctgg ggcaacctac |
| 3721 | taatcctctc tgcaagtcgg tctccttatc cccccaaatg |
| | gaaattgtat ttgccttctc |
| 3781 | cactttggga ggctcccact tcttgggagg gttacatttt |
| | ttaagtctta atcatttgtg |
| 3841 | acatatgtat ctatacatcc gtatctttta atgatccgtg |
| | tgtaccatct ttgtgattat |
| 3901 | ttccttaata ttttttcttt aagtcagttc attttcgttg |
| | aaatacattt atttaaagaa |
| 3961 | aaatctttgt tactctgtaa atgaaaaaac ccattttcgc |
| | tataaataaa aggtaactgt |
| 4021 | acaaaataag tacaatgcaa caaaaaaaaa |

An exemplary human FASLG amino acid sequence is set forth below (SEQ ID NO: 186; GenBank Accession No: AAH17502.1, Version 1, incorporated herein by reference):

| | |
|---|---|
| 1 | mqqpfnypyp qiywvdssas spwappgtvl pcptsvprrp |
| | gqrrpppppp ppplpppppp |
| 61 | pplpplplpp lkkrgnhstg lcllvmffmv lvalvglglg |
| | mfqlfhlqke laelrestsq |
| 121 | mhtasslekq ighpspppek kelrkvahlt gksnsrsmpl |
| | ewedtygivl lsgvkykkgg |
| 181 | lvinetglyf vyskvyfrgq scnnlplshk vymrnskypq |
| | dlvmmegkmm sycttgqmwa |
| 241 | rssylgavfn ltsadhlyvn vselslvnfe esqtffglyk l |

An exemplary human FASLG nucleic acid sequence is set forth below (SEQ ID NO: 187; GenBank Accession No: NM_000639.2, Version 2, incorporated herein by reference):

| | |
|---|---|
| 1 | agaatcagag agagagagat agagaaagag aaagacagag |
| | gtgtttccct tagctatgga |
| 61 | aactctataa gagagatcca gcttgcctcc tcttgagcag |
| | tcagcaacag ggtcccgtcc |
| 121 | ttgacacctc agcctctaca ggactgagaa gaagtaaaac |
| | cgtttgctgg ggctggcctg |
| 181 | actcaccagc tgccatgcag cagcccttca attacccata |
| | tccccagatc tactgggtgg |
| 241 | acagcagtgc cagctctccc tgggcccctc caggcacagt |
| | tcttccctgt ccaacctctg |
| 301 | tgcccagaag gcctggtcaa aggaggccac caccaccacc |
| | gccaccgcca ccactaccac |
| 361 | ctccgccgcc gccgccacca ctgcctccac taccgctgcc |
| | acccctgaag aagagaggga |
| 421 | accacagcac aggcctgtgt ctccttgtga tgtttttcat |
| | ggttctggtt gccttggtag |
| 481 | gattgggcct ggggatgttt cagctcttcc acctacagaa |
| | ggagctggca gaactccgag |
| 541 | agtctaccag ccagatgcac acagcatcat ctttggagaa |
| | gcaaataggc caccccagtc |
| 601 | caccccctga aaaaaaggag ctgaggaaag tggcccattt |
| | aacaggcaag tccaactcaa |
| 661 | ggtccatgcc tctggaatgg gaagacacct atggaattgt |
| | cctgctttct ggagtgaagt |
| 721 | ataagaaggg tggccttgtg atcaatgaaa ctgggctgta |
| | ctttgtatat tccaaagtat |
| 781 | acttccgggg tcaatcttgc aacaacctgc ccctgagcca |
| | caaggtctac atgaggaact |
| 841 | ctaagtatcc ccaggatctg gtgatgatgg aggggaagat |
| | gatgagctac tgcactactg |
| 901 | ggcagatgtg ggcccgcagc agctacctgg gggcagtgtt |
| | caatcttacc agtgctgatc |
| 961 | atttatatgt caacgtatct gagctctctc tggtcaattt |
| | tgaggaatct cagacgtttt |
| 1021 | tcggcttata taagctctaa gagaagcact ttgggattct |
| | ttccattatg attctttgtt |
| 1081 | acaggcaccg agaatgttgt attcagtgag ggtcttctta |
| | catgcatttg aggtcaagta |
| 1141 | agaagacatg aaccaagtgg accttgagac cacagggttc |
| | aaaatgtctg tagctcctca |
| 1201 | actcacctaa tgtttatgag ccagacaaat ggaggaatat |
| | gacggaagaa catagaactc |
| 1261 | tgggctgcca tgtgaagagg gagaagcatg aaaaagcagc |
| | taccaggtgt tctacactca |
| 1321 | tcttagtgcc tgagagtatt taggcagatt gaaaaggaca |
| | ccttttaact cacctctcaa |
| 1381 | ggtgggcctt gctacctcaa gggggactgt ctttcagata |
| | catggttgtg acctgaggat |
| 1441 | ttaagggatg gaaaaggaag actagaggct tgcataataa |
| | gctaaagagg ctgaaagagg |
| 1501 | ccaatgcccc actggcagca tcttcacttc taaatgcata |
| | tcctgagcca tcggtgaaac |
| 1561 | taacagataa gcaagagaga tgttttgggg actcatttca |
| | ttcctaacac agcatgtgta |
| 1621 | tttccagtgc aattgtaggg gtgtgtgtgt gtgtgtgtgt |
| | gtgtgtgtgt gtatgactaa |
| 1681 | agagagaatg tagatattgt gaagtacata ttaggaaaat |
| | atgggttgca tttggtcaag |
| 1741 | attttgaatg cttcctgaca atcaactcta atagtgctta |
| | aaaatcattg attgtcagct |
| 1801 | actaatgatg ttttcctata atataataaa tatttatgta |
| | gatgtgcatt tttgtgaaat |
| 1861 | gaaaacatgt aataaaaagt atatgttagg atacaaaaaa |
| | aaaaaaa |

An exemplary human SLAMF6 amino acid sequence is set forth below (SEQ ID NO: 188; GenBank Accession No: AAI14496.1, Version 1, incorporated herein by reference):

| | |
|---|---|
| 1 | mlwlfqsllf vfcfgpgnvv sqssltplmv ngilgesvtl |
| | plefpagekv nfitwlfnet |
| 61 | slafivphet kspeihvtnp kqgkrinftq syslqlsnlk |
| | medtgsyraq istktsakls |
| 121 | sytlrilrql rniqvtnhsq lfqnmtcelh ltcsvedadd |
| | nvsfrwealg ntlssqpnlt |
| 181 | vswdprisse qdytciaena vsnlsfsysa qklcedvkiq |
| | ytdtkmilfm vsgicivfgf |
| 241 | iillllvlrk rrdslslstq rtqgpaesar nleyvsyspt |
| | nntvyasvth snreteiwtp |
| 301 | rendtitiys tinhskeskp tfsrataldn vv |

An exemplary human SLAMF6 nucleic acid sequence is set forth below (SEQ ID NO: 189; GenBank Accession No: NM_001184714.1, Version 1, incorporated herein by reference):

| | |
|---|---|
| 1 | agtttatgac agaagggcaa aaacattgac tgcctcaagg |
| | tctcaagcac cagtcttcac |
| 61 | cgcggaaagc atgttgtggc tgttccaatc gctcctgttt |
| | gtcttctgct ttggcccagg |
| 121 | gaatgtagtt tcacaaagca gcttaacccc attgatggtg |
| | aacgggattc tgggggagtc |
| 181 | agtaactctt cccctggagt ttcctgcagg agagaaggtc |
| | aacttcatca cttggctttt |
| 241 | caatgaaaca tctcttgcct tcatagtacc ccatgaaacc |
| | aaaagtccag aaatccacgt |
| 301 | gactaatccg aaacagggaa agcgactgaa cttcacccag |
| | tcctactccc tgcaactcag |
| 361 | caacctgaag atggaagaca caggctctta cagagcccag |
| | atatccacaa agacctctgc |
| 421 | aaagctgtcc agttacactc tgaggatatt aagacaactg |
| | aggaacatac aagttaccaa |
| 481 | tcacagtcag ctatttcaga atatgacctg tgagctccat |
| | ctgacttgct ctgtggagga |
| 541 | tgcagatgac aatgtctcat tcagatggga ggccttggga |
| | aacacacttt caagtcagcc |
| 601 | aaacctcact gtctcctggg accccaggat ttccagtgaa |
| | caggactaca cctgcatagc |
| 661 | agagaatgct gtcagtaatt tatccttctc tgtctctgcc |
| | cagaagcttt gcgaagatgt |
| 721 | taaaattcaa tatacagata ccaaaatgat tctgtttatg |
| | gtttctggga tatgcatagt |
| 781 | cttcggtttc atcatactgc tgttacttgt tttgaggaaa |
| | agaagagatt ccctatcttt |
| 841 | gtctactcag cgaacacagg gccccgcaga gtccgcaagg |
| | aacctagagt atgtttcagt |
| 901 | gtctccaacg aacaacactg tgtatgcttc agtcactcat |
| | tcaaacaggg aaacagaaat |
| 961 | ctggacacct agagaaaatg atactatcac aatttactcc |
| | acaattaatc attccaaaga |
| 1021 | gagtaaaccc actttttcca gggcaactgc ccttgacaat |
| | gtcgtgtaag ttgctgaaag |
| 1081 | gcctcagagg aattcgggaa tgacacgtct tctgatccca |
| | tgagacagaa caaagaacag |
| 1141 | gaagcttggt tcctgttgtt cctggcaaca gaatttgaat |
| | atctaggata ggatgatcac |
| 1201 | ctccagtcct tcggacttaa acctgcctac ctgagtcaaa |
| | cacctaagga taacatcatt |
| 1261 | tccagcatgt ggttcaaata atattttcca atccacttca |
| | ggccaaaaca tgctaaagat |
| 1321 | aacacaccag cacattgact ctctctttga taactaagca |
| | aatggaatta tggttgacag |
| 1381 | agagtttatg atccagaaga caaccacttc tctcctttta |
| | gaaagcagca ggattgactt |
| 1441 | attgagaaat aatgcagtgt gttggttaca tgtgtagtct |
| | ctggagttgg atgggcccat |
| 1501 | cctgatacaa gttgagcatc ccttgtctga aatgcttggg |
| | attagaaatg tttcagattt |
| 1561 | caattttttt tcagattttg gaatatttgc attatattta |
| | gcggttgagt atccaaatcc |
| 1621 | aaaaatccaa aattcaaaat gctccaataa gcatttccct |
| | tgagtttcat tgatgtcgat |
| 1681 | gcagtgctca aaatctcaga ttttggagca ttttggatat |
| | tggatttttg gatttgggat |
| 1741 | gctcaacttg tacaatgttt attagacaca tctcctggga |
| | catactgcct aaccttttgg |
| 1801 | agccttagtc tcccagactg aaaaaggaag aggatggtat |
| | tacatcagct ccattgtttg |
| 1861 | agccaagaat ctaagtcatc cctgactcca gtgtctttgt |
| | caccaggccc tttggactct |
| 1921 | acctcagaaa tatttcttgg accttccact tctcctccaa |
| | ctccttgacc accatcctgt |
| 1981 | atccaaccat caccacctct aacctgaatc ctaccttaag |
| | atcagaacag ttgtcctcac |
| 2041 | ttttgttctt gtccctctcc aacccactct ccacaagatg |
| | gccagagtaa tgtttttaat |
| 2101 | ataaattgga tccttcagtt tcctgcttaa aaccctgcag |
| | gtttcccaat gcactcagaa |
| 2161 | agaaatccag tttccatggc cctggatggt ctggcccacc |
| | tccagcctca gctagcatta |
| 2221 | cccttctgac actctctatg tagcctccct gatcttcttt |
| | cagctcctct attaaaggaa |
| 2281 | aagttcttta tgttaattat ttacatcttc ctgcaggccc |
| | ttcctctgcc tgctggggtc |
| 2341 | ctcctattct ttaggtttaa ttttaaatat gtcacctcct |
| | aagagaaacc ttcccagacc |
| 2401 | actctttcta aaatgaatct tctaggctgg gcatggtggc |
| | tcacacctgt aatccctgta |
| 2461 | ctttgggagg ccaagggggg agatcacttg aggtcaggag |
| | ttcaagacca gcctggccaa |
| 2521 | cttggtgaaa ccccgtcttt actaaaaata caaaaaaatt |
| | agccaggcgt ggtggtgcac |
| 2581 | ccctaaaatc ccagctactt gagagactga ggcaggagaa |
| | tcgcttgaac ccaggaggtg |
| 2641 | gaggttccag tgagccaaaa tcatgccaat gtattccagt |
| | ctgggtgaca gagtgagact |
| 2701 | ctgtctcaaa aaataaataa ataaaataaa atgaaataga |
| | tcttataaaa aaaa |

An exemplary human GAPDH amino acid sequence is set forth below (SEQ ID NO: 190; GenBank Accession No: NP_001276675.1, Version 1, incorporated herein by reference):

| | |
|---|---|
| 1 | mgkvkvgvng fgrigrlvtr aafnsgkvdi vaindpfidl |
| | nymvymfqyd sthgkfhgtv |
| 61 | kaengklvin gnpitifqer dpskikwgda gaeyvvestg |
| | vfttmekaga hlqggakrvi |
| 121 | isapsadapm fvmgvnheky dnslkiisna scttnclapl |
| | akvihdnfgi veglmttvha |
| 181 | itatqktvdg psgklwrdgr galqniipas tgaakavgkv |
| | ipelngkltg mafrvptanv |
| 241 | svvdltcrle kpakyddikk vvkqasegpl kgilgytehq |
| | vvssdfnsdt hsstfdagag |
| 301 | ialndhfvkl iswydnefgy snrvvdlmah maske |

An exemplary human GAPDH nucleic acid sequence is set forth below (SEQ ID NO: 191; GenBank Accession No: NM_002046.5, Version 5, incorporated herein by reference):

| | |
|---|---|
| 1 | gcctcaagac cttgggctgg gactggctga gcctggcggg |
| | aggcggggtc cgagtcaccg |
| 61 | cctgccgccg cgcccccggt ttctataaat tgagcccgca |
| | gcctcccgct tcgctctctg |
| 121 | ctcctcctgt tcgacagtca gccgcatctt cttttgcgtc |
| | gccagccgag ccacatcgct |
| 181 | cagacaccat ggggaaggtg aaggtcggag tcaacggatt |
| | tggtcgtatt gggcgcctgg |
| 241 | tcaccagggc tgcttttaac tctggtaaag tggatattgt |
| | tgccatcaat gaccccttca |
| 301 | ttgacctcaa ctacatggtt tacatgttcc aatatgattc |
| | cacccatggc aaattccatg |
| 361 | gcaccgtcaa ggctgagaac gggaagcttg tcatcaatgg |
| | aaatcccatc accatcttcc |
| 421 | aggagcgaga tccctccaaa atcaagtggg gcgatgctgg |
| | cgctgagtac gtcgtggagt |
| 481 | ccactggcgt cttcaccacc atggagaagg ctggggctca |
| | tttgcagggg ggagccaaaa |
| 541 | gggtcatcat ctctgccccc tctgctgatg cccccatgtt |
| | cgtcatgggt gtgaaccatg |
| 601 | agaagtatga caacagcctc aagatcatca gcaatgcctc |
| | ctgcaccacc aactgcttag |
| 661 | cacccctggc caaggtcatc catgacaact ttggtatcgt |
| | ggaaggactc atgaccacag |
| 721 | tccatgccat cactgccacc cagaagactg tggatggccc |
| | ctccgggaaa ctgtggcgtg |
| 781 | atggccgcgg ggctctccag aacatcatcc ctgcctctac |
| | tggcgctgcc aaggctgtgg |
| 841 | gcaaggtcat ccctgagctg aacgggaagc tcactggcat |
| | ggccttccgt gtccccactg |
| 901 | ccaacgtgtc agtggtggac ctgacctgcc gtctagaaaa |
| | acctgccaaa tatgatgaca |
| 961 | tcaagaaggt ggtgaagcag gcgtcggagg gccccctcaa |
| | gggcatcctg ggctacactg |
| 1021 | agcaccaggt ggtctcctct gacttcaaca gcgacaccca |
| | ctcctccacc tttgacgctg |
| 1081 | gggctggcat tgccctcaac gaccactttg tcaagctcat |
| | ttcctggtat gacaacgaat |
| 1141 | ttggctacag caacagggtg gtggacctca tggcccacat |
| | ggcctccaag gagtaagacc |
| 1201 | cctggaccac cagccccagc aagagcacaa gaggaagaga |
| | gagaccctca ctgctgggga |
| 1261 | gtccctgcca cactcagtcc cccaccacac tgaatctccc |
| | ctcctcacag ttgccatgta |
| 1321 | gaccccttga agaggggagg ggcctaggga gccgcacctt |
| | gtcatgtacc atcaataaag |
| 1381 | taccctgtgc tcaaccagtt aaaaaaaaaa aaaaaaaaaa |
| | a |

An exemplary human HPRT1 amino acid sequence is set forth below (SEQ ID NO: 192; GenBank Accession No: AAH00578.1, Version 1, incorporated herein by reference):

| | |
|---|---|
| 1 | matrspgvvi sddepgydld lfcipnhyae dlervfiphg |
| | limdrterla rdvmkemggh |
| 61 | hivalcvlkg gykffadlld yikalnrnsd rsipmtvdfi |
| | rlksycndqs tgdikviggd |
| 121 | dlstltgknv livediidtg ktmqtllslv rqynpkmvkv |
| | asllvkrtpr svgykpdfvg |
| 181 | feipdkfvvg yaldyneyfr dlnhvcvise tgkakyka |

An exemplary human HPRT1 nucleic acid sequence is set forth below (SEQ ID NO: 193; GenBank Accession No: NM_000194.2, Version 2, incorporated herein by reference):

| | |
|---|---|
| 1 | ggcggggcct gcttctcctc agcttcaggc ggctgcgacg |
| | agccctcagg cgaacctctc |
| 61 | ggctttcccg cgcggcgccg cctcttgctg cgcctccgcc |
| | tcctcctctg ctccgccacc |
| 121 | ggcttcctcc tcctgagcag tcagcccgcg cgccggccgg |
| | ctccgttatg gcgacccgca |
| 181 | gccctggcgt cgtgattagt gatgatgaac caggttatga |
| | ccttgattta ttttgcatac |
| 241 | ctaatcatta tgctgaggat ttggaaaggg tgtttattcc |
| | tcatggacta attatggaca |
| 301 | ggactgaacg tcttgctcga gatgtgatga aggagatggg |
| | aggccatcac attgtagccc |
| 361 | tctgtgtgct caaggggggc tataaattct ttgctgacct |
| | gctggattac atcaaagcac |
| 421 | tgaatagaaa tagtgataga tccattccta tgactgtaga |
| | ttttatcaga ctgaagagct |
| 481 | attgtaatga ccagtcaaca ggggacataa aagtaattgg |
| | tggagatgat ctctcaactt |
| 541 | taactggaaa gaatgtcttg attgtggaag atataattga |
| | cactggcaaa acaatgcaga |
| 601 | ctttgctttc cttggtcagg cagtataatc caaagatggt |
| | caaggtcgca agcttgctgg |
| 661 | tgaaaaggac cccacgaagt gttggatata agccagactt |
| | tgttggattt gaaattccag |
| 721 | acaagtttgt tgtaggatat gcccttgact ataatgaata |
| | cttcagggat ttgaatcatg |
| 781 | tttgtgtcat tagtgaaact ggaaaagcaa aatacaaagc |
| | ctaagatgag agttcaagtt |
| 841 | gagtttggaa acatctggag tcctattgac atcgccagta |
| | aaattatcaa tgttctagtt |
| 901 | ctgtggccat ctgcttagta gagctttttg catgtatctt |
| | ctaagaattt tatctgtttt |
| 961 | gtactttaga aatgtcagtt gctgcattcc taaactgttt |
| | atttgcacta tgagcctata |
| 1021 | gactatcagt tccctttggg cggattgttg tttaacttgt |
| | aaatgaaaaa attctcttaa |
| 1081 | accacagcac tattgagtga aacattgaac tcatatctgt |
| | aagaaataaa gagaagatat |
| 1141 | attagttttt taattggtat tttaattttt atatatgcag |
| | gaaagaatag aagtgattga |
| 1201 | atattgttaa ttataccacc gtgtgttaga aaagtaagaa |
| | gcagtcaatt ttcacatcaa |
| 1261 | agacagcatc taagaagttt tgttctgtcc tggaattatt |
| | ttagtagtgt ttcagtaatg |
| 1321 | ttgactgtat tttccaactt gttcaaatta ttaccagtga |
| | atctttgtca gcagttccct |
| 1381 | tttaaatgca aatcaataaa ttcccaaaaa tttaaaaaaa |
| | aaaaaaaaaa aaaaa |

An exemplary human PSK1 amino acid sequence is set forth below (SEQ ID NO: 194; GenBank Accession No: NP_079418.1, Version 1, incorporated herein by reference):

| | |
|---|---|
| 1 | mpstnragsl kdpeiaelff kedpeklftd lreighgsfg |
| | avyfardvrt nevvaikkms |
| 61 | ysgkqstekw qdiikevkfl qrikhpnsie ykgcylreht |
| | awlvmeyclg sasdllevhk |
| 121 | kplqeveiaa ithgalqgla ylhshtmihr dikagnillt |
| | epgqvkladf gsasmaspan |
| 181 | sfvgtpywma pevilamdeg qydgkvdvws lgitcielae |
| | rkpplfnmna msalyhiaqn |
| 241 | esptlqsnew sdyfrnfvds clqkipqdrp tseellkhif |
| | vlrerpetvl idliqrtkda |
| 301 | vreldnlqyr kmkkllfqea hngpaveaqe eeeeqdhgvg |
| | rtgtvnsvgs nqsipsmsis |
| 361 | assqsssvns lpdvsddkse ldmmegdhtv msnssvihlk |
| | peeenyreeg dprtrasdpq |
| 421 | sppqvsrhks hyrnrehfat irtaslvtrq mqeheqdsel |
| | reqmsgykrm rrqhqkqlmt |
| 481 | lenklkaemd ehrlrldkdl etqrnnfaae meklikkhqa |
| | amekeakvms neekkfqqhi |
| 541 | qaqqkkelns flesqkreyk lrkeqlkesk elqikkqfqd |
| | tckiqtrqyk alrnhllett |
| 601 | pksehkavlk rlkeeqtrkl ailaeqydhs inemlstqal |
| | rldeaqeaec qvlkmqlqqe |
| 661 | lellnayqsk ikmqaeaqhd relreleqrv slrralleqk |
| | ieeemlalqn erterirsll |
| 721 | erqareieaf dsesmrlgfs nmvlsnlspe afshsypgas |
| | gwshnptggp gphwghpmgg |
| 781 | ppqawghpmq ggpqpwghps gpmqgvprgs smgvrnspqa |
| | lrrtasggrt eqgmsrstsv |
| 841 | tsqisngshm syt |

An exemplary human PSK1 nucleic acid sequence is set forth below (SEQ ID NO: 195; GenBank Accession No: NM_025142.1, Version 1, incorporated herein by reference):

| | |
|---|---|
| 1 | cccctcctcc tcctcactcc tcaccctcca gggtagcggc |
| | taccggagcg ctgcaggggg |
| 61 | ctgcgcctgc ctgctccgcc ccagacctgt cggcgaaagg |
| | tagtttatgc caacgtgact |
| 121 | tcattcatac agatgaacca aggatcggga tagcagtata |
| | aaattagaat caagacagct |
| 181 | gactgctcag caggatgcca tcaactaaca gagcaggcag |
| | cctgaaggac cctgaaattg |
| 241 | cagagctctt cttcaaagaa gatccagaga agctcttcac |
| | agatctcaga gaaattggcc |
| 301 | atggaagctt tggagcagtg tattttgcac gagatgtgcg |
| | taccaatgaa gtggtggcca |
| 361 | tcaagaaaat gtcttatagt ggaaagcagt ctactgagaa |
| | atggcaggat attattaagg |
| 421 | aagtcaagtt tctacaaaga ataaaacatc ccaacagtat |
| | agaatacaaa ggctgttatt |
| 481 | tacgtgaaca cacagcatgg cttgtaatgg aatattgttt |
| | aggatctgct tcggatttac |
| 541 | tagaagttca caaaaagcca ttacaagaag tggaaatagc |
| | agcaattaca catggtgctc |
| 601 | ttcagggatt agcctactta cattctcata ctatgattca |
| | tagagatatc aaagcaggaa |
| 661 | atatccttct gacagaacca ggccaggtga aacttgctga |
| | ctttggctct gcttccatgg |
| 721 | catcacctgc caattccttt gtgggaacgc cgtattggat |
| | ggccccagaa gtaattttag |
| 781 | ccatggatga aggacaatat gatggcaaag tagatgtgtg |
| | gtctcttgga ataacatgta |
| 841 | ttgaactagc ggaaaggaag cctcctttat ttaatatgaa |
| | tgcaatgagt gccttatatc |
| 901 | acatagccca aaatgaatcc cctacactac agtctaatga |
| | atggtctgat tattttcgca |
| 961 | actttgtaga ttcttgcctc cagaaaatcc ctcaagatcg |
| | acctacatca gaggaacttt |
| 1021 | taaagcacat atttgttctt cgggagcgcc ctgaaaccgt |
| | gttaatagat ctcattcaga |
| 1081 | ggacaaagga tgcagtaaga gagctggaca atctgcagta |
| | tcgaaagatg aagaaactcc |
| 1141 | ttttccagga ggcacataat ggaccagcag tagaagcaca |
| | ggaagaagaa gaggaacaag |
| 1201 | atcatggtgt tggccggaca ggaacagtta atagtgttgg |
| | aagtaatcaa tccattccca |
| 1261 | gcatgtccat cagtgccagc agccaaagca gtagtgttaa |
| | cagtcttcca gatgtctcag |
| 1321 | atgacaagag tgagctagac atgatggagg gagaccacac |
| | agtgatgtct aacagttctg |
| 1381 | ttatccattt aaaaccagag gaagaaaatt acagagaaga |
| | gggagatcct agaacaagag |
| 1441 | catcagatcc acaatctcca ccccaagtat ctcgtcacaa |
| | atcacactat cgtaatcgag |
| 1501 | aacactttgc tactatacgg acagcatcac tggttacgag |
| | gcaaatgcaa gaacatgagc |
| 1561 | aggactctga gcttagagaa caaatgtctg gctataagcg |
| | aatgaggcga caacatcaaa |
| 1621 | agcaactgat gactctggaa aacaagctaa aggctgagat |
| | ggatgaacat cgcctcagat |
| 1681 | tagacaaaga tcttgaaact cagcgtaaca attttgctgc |
| | agaaatggag aaacttatca |
| 1741 | agaaacacca ggctgccatg gagaaagagg ctaaagtgat |
| | gtccaatgaa gagaaaaaat |
| 1801 | ttcagcaaca tattcaggcc caacagaaga aagaactgaa |
| | tagttttctc gagtcccaga |
| 1861 | aaagagagta taaacttcga aaagagcagc ttaaagagtc |
| | taaagaactc caaataaaaa |
| 1921 | agcagtttca ggatacctgc aaaatccaaa ccagacagta |
| | caaagcatta agaaatcacc |
| 1981 | tgctggagac tacaccaaag agtgagcaca aagctgttct |
| | gaaacggctc aaggaggaac |
| 2041 | agacccggaa attagctatc ttggctgagc agtatgatca |
| | cagcattaat gaaatgctct |
| 2101 | ccacacaagc cctgcgtttg gatgaagcac aggaagcaga |
| | gtgccaggtt ttgaagatgc |
| 2161 | agctgcagca ggaactggag ctgttgaatg cgtatcagag |
| | caaaatcaag atgcaagctg |
| 2221 | aggcacaaca tgatcgagag cttcgcgagc ttgaacagag |
| | ggtctccctc cggagggcac |
| 2281 | tcttagaaca aaagattgaa gaagagatgt tggctttgca |
| | gaatgagcgc acagaacgaa |
| 2341 | tacgaagcct gttggaacgt caagccagag agattgaagc |
| | ttttgactct gaaagcatga |
| 2401 | gactaggttt tagtaatatg gtcctttcta atctctcccc |
| | tgaggcattc agccacagct |
| 2461 | acccgggagc ttctggttgg tcacacaacc ctactggggg |
| | tccaggacct cactggggtc |
| 2521 | atcccatggg tggcccacca caagcttggg gccatccaat |
| | gcaaggtgga ccccagccat |
| 2581 | ggggtcaccc ttcagggcca atgcaagggg tacctcgagg |
| | tagcagtatg ggagtccgca |
| 2641 | atagccccca ggctctgagg cggacagctt ctgggggacg |
| | gacggagcag ggcatgagca |
| 2701 | gaagcacgag tgtcacttca caaatatcca atgggtcaca |
| | catgtcttat acataactta |
| 2761 | ataattgaga gtggcaattc cgctggagct gtctgccaaa |
| | agaaactgcc tacagacatc |
| 2821 | atcacagcag cctcctcact tgggtactac agtgtggaag |
| | ctgagtgcat atggtatatt |
| 2881 | ttattcattt ttgtaaagcg ttctgttttg tgtttactaa |
| | ttgggatgtc atagtacttg |
| 2941 | gctgccgggt ttgtttgttt ttggggaaat tttgaaaagt |
| | ggagttgata ttaaaaataa |
| 3001 | atgtgtatgt gtgtacatat atatacacac acatacacat |
| | atattatgca tgtggtgaaa |
| 3061 | agaattggct agatagggga tttttctgaa cactgcaaaa |
| | atagaacgta gcaaaatggc |
| 3121 | ttcagttatc acttttgggt gtctgtatcc taagaagttt |
| | ctgaaaagat ctaaagcctt |
| 3181 | tttatcccat atcccaaatt cttatgagcc actcacagca |
| | ggcagcatat gttgaaataa |
| 3241 | gttattactg gtacacacct gcattgcctc accagtgtat |
| | ttatttgtta ttaaattgat |
| 3301 | ctgacttctc agcctcattt ggactaaaaa aagaaagcag |
| | aaatccatga acacattgct |
| 3361 | tctcggcctt ttggctaaga tcaagtgtag aaatccatga |
| | acactaaagg acttcattga |
| 3421 | ttttttcaga gagtagaaaa caacttagtt tttctttttt |
| | cctgaatgcg tcataggctt |
| 3481 | gtgagtgatt tttgtccatt caattgtgcc ttctttgtat |
| | tatgataaga tgggggtact |
| 3541 | taaggagatc acaagttgtg tgaggattgc attaacaaac |
| | ctatgagcct tcaatgggga |
| 3601 | agaccagaag ggtgagaggg gccctgaaag ttcatatggt |
| | gggtatgtcc cgcagcagag |
| 3661 | tgaggagatg aagcttacgt gtcctgacgt tttgttgctt |
| | atactgtgat atctcatcct |
| 3721 | agctaagctc tataatgccc aagaccccaa acagtacttt |
| | tactttgttt gtacaaaaac |
| 3781 | aaagacatat agccaataca aatcaaatgc cggaggtgtt |
| | tgatgccata tttgcaaatt |
| 3841 | gccatctatt gaaattctcg tcacactaca tagacataat |
| | tgttatctcc ttttggctta |
| 3901 | tgtgattttc tgtttacaag tagaatagcc aattatttaa |
| | atgtttagtt gccacagtga |
| 3961 | accaggagtc actgagccaa tgactttacc agctgctgac |
| | taatcttcat caccactgta |
| 4021 | gattttgctg catgtgcagg tcctctattt ttaattgctg |
| | ttttcgttgc tgcagtactt |
| 4081 | tacaaacttc tagttcgttg agacttagtg accatttggc |
| | atcaagttaa catcacacaa |
| 4141 | taggaaacac cacttccaca agtctcaagc ctcagtgcta |
| | aagtactact gaaaaggaac |
| 4201 | taggaagttt ggccaattaa aaaaaaaaag taaaataagt |
| | tatagtggtc agggaatctc |
| 4261 | ttgacgaaag ctaactctta tttttcaggg ggcacatgtt |
| | ttgttttgtt atgttttgtt |
| 4321 | gtacaatgaa ggatgaaccc aatgctaaaa agtcagttat |
| | tttcttggtt ttccatttct |
| 4381 | tgtatataaa ttgtgttgga ggtgggtagg aagctgtgag |
| | tatgacttga agaaaaatat |
| 4441 | ccttttcagt gacaaatcag agtttcttac aagttattgt |
| | cctgctccct tccaagttgt |
| 4501 | cttgaagaat ccttgctgct aactctggat cctgcttttc |
| | ctatagtcag agggctccaa |
| 4561 | ggtagaactc tctaagtccc tctcaatggc actctttgcc |
| | tagaccaaac taatgaccaa |
| 4621 | acagtaatca atctatcact gttgaagtcc tggttttctc |
| | aaccaaattt taagtctttc |
| 4681 | ccaggtatgt cagtcgagtt gccatgaatc ctcacctgta |
| | ggagtttctg ggattcattt |
| 4741 | taaggaaact ggaagaaaat taatgtttta atgtaaatgt |
| | ttttaaaacc aagatcacca |
| 4801 | tagagttcac acaaaatttt aggcagtgtt tcaagaagca |
| | ctcttatgtg ttgttctttc |
| 4861 | agcccatgct ccaggtcacc ggtttttagg taaacatgac |
| | atgacatgta cttgtaatat |
| 4921 | cagtgaaagg ggcttttagt cgtctcagta tcttttttgc |
| | attcaggtat tatagcgttt |
| 4981 | ccaacaaatt gagaaattac ttatttggtg taaaatgacg |
| | agagagcttt agaatttaat |
| 5041 | aaaatgtcaa aaatacaatt tgataccctt aaaaattatt |
| | catagatctg gctgctctgt |
| 5101 | actactggat gggtggaata gggaagcaga tcacttttgc |
| | atacaaaaac tctttcaaac |
| 5161 | ccaaaatatt attggtggta cattttaaag tccaattgtg |
| | gacttaaatt agtcaaattg |
| 5221 | agagttactg agtccattca gatctccagt agggttttgt |
| | atctaatgta tttggccacc |
| 5281 | agttttacat gtaggtcatg actatacctg gattttatca |
| | ttaagttaac tttaaagtat |
| 5341 | atacagtttg caaaaaatga ttcaaattaa ttggtttgta |
| | tatgtttgtg agatctagct |
| 5401 | tctgaggaat ctcatacata gtaggaacca tcatagagta |
| | aagcttgtag cagaaaaatt |
| 5461 | ggaagggttt tgagatatcc tagagaaaga gcaagcactt |
| | tctgaatcag tggggaaata |
| 5521 | tcaatatttg atcaaattaa cactacctcc ttcccagtgt |
| | tggtgttcac tcactatatg |
| 5581 | tctttaagaa aattaaaact atggaaaatt tgtctagcat |
| | atcagaagtt gtaaatgcta |
| 5641 | tatctggtat ccagaggctg gctgtaaaaa gttccttggg |
| | gtcactctat ctcacatttt |
| 5701 | ttttggttag cattttaaaa atgcaaagcc acatactttg |
| | aaatatatta ttccaaattg |
| 5761 | agctcccttc cctttgcaca tattttttcc tccccttatt |
| | gaagtcagct ctaaccccaa |
| 5821 | attctagtat ccaaaagtat ttttatttgt ataatgctat |
| | ctgaaaaatg tgtttatatt |
| 5881 | atattttcag agctgcaatt cttattcgcc atttcaatac |
| | ctagagatag agagtcttgt |
| 5941 | atttgaagcc acacacactg gtgtaatatg cttagtactc |
| | taggtcaagg atttgttggt |
| 6001 | aaatggaaca ttttagcata gtcatgattt ttggttgcct |
| | agacatcagg taaacattca |
| 6061 | gtacactaaa gaaactatcc tggacactcc ctcctgcttc |
| | caccaatttt tttctcaccc |
| 6121 | ccttttcaaa aattgaaaac tctatgagtg tctttttgag |
| | accataaagc agactttagt |
| 6181 | aactttctat ttctgtaagt actaaatgtc tggcatttta |
| | aacttttgta gaatacatta |
| 6241 | tgttggacac tggaataata ctatttattt tcacctgtga |
| | aaaatgactt cattgtactt |
| 6301 | gaaacacctc ctttgcattt ctccatttgt gccattcact |
| | agtggaaata aattgtatta |
| 6361 | taccatgatc tactggcttt ttaaaactgt attaaatatg |
| | cacatttttg gtatagctat |
| 6421 | tatcatttgt atgtatatat tgtatataca tatgagtgtc |
| | tgtatgtgtg tatagatgga |
| 6481 | tggatgtaac tcatactgta catttccatc agggcactta |
| | aaagttctgt tatttttgtt |
| 6541 | tggttttgtt atttcagtcc tcagttaagg caagaatgca |
| | tgtgtttctt aagaatgagt |
| 6601 | actctgcgtt gatgtttatg agaaggtggt cattagatgc |
| | agtcttttcc tttttaatcc |
| 6661 | cctcttagca cttctgtgag tggagaggac attaagtaaa |
| | atttggaatc ataagttgca |
| 6721 | atgcagtaaa atggtgctgg ggaaggagcc agttagtgtt |
| | tctgtgagtt tgtgttgtga |
| 6781 | tgcaataaga gataagtaat gcagagagaa atgaaccatg |
| | gaaagtaaga acactgatgg |
| 6841 | tgattcctct gcaaagatga taagaaaaag aaccaataaa |
| | tcacacaatc tttatgtgct |
| 6901 | ttctatatgt atttcttagt agtgatacca ttgatcctct |
| | tacttttttt actccattaa |
| 6961 | tactaataat tatatacttt gctgaggatc aaaacagcca |
| | agaaaggaat tactgctaaa |
| 7021 | gcatctaaga ttctcctgaa ctgtaaaatc aacaggaaat |
| | ggccactggg agagaaggat |
| 7081 | ttggtattgg gtgaggggct ttctcccttt acctgcctct |
| | tcttgcttgc taatagtaag |
| 7141 | ttctttgtgc accttccacc acttctgagc cactactatt |
| | caagtagaga tttgccccaa |
| 7201 | cacattaact ttttccttgg agatttatat ggtcctgcat |
| | tttgtcctgt gctcacaatg |
| 7261 | tgaagtgtct tctgtattca aatcaaaaaa taatatattt |
| | aaggtatata agtgtgaatc |
| 7321 | tcctataatg atggaagaag aggttctctt gtcttagata |
| | gaaaagagcc ttctccaaga |
| 7381 | gcaatgtcaa aacttgggct gtcatctttg agctgtttac |
| | caaaatacag accattattg |
| 7441 | aagaaaaaca aattatctat tttgttttcc cccatctaac |
| | atgatagtgc ccccaaccag |
| 7501 | gttgtagcat tgccttttaa aagagactca ctcactctta |
| | gtttttaaga actggaaatt |
| 7561 | tcccatcctc agatccctta aaggatgaag agttggctgt |
| | acacttagcg gacttgcctc |
| 7621 | ttgtatgcaa ggactactga ttgaagtctg ttttgctgtg |
| | tctggttatg ttgtctgcac |
| 7681 | ttttatgaaa tcactacaat aggtctgcat tggaaatgac |
| | tattaatttg taaagaagta |
| 7741 | agttttatta aacactgtct agaaaaagaa agtgaagctg |
| | agaactcttc ctttattgtg |
| 7801 | catttatatt ttctgctgaa ttccggtagt tccctttaaa |
| | gtcatgttga ctaatgtttt |
| 7861 | cctccttgtt tgtattcaga tttccaaaat ttcactcata |
| | caagggaaga gactccattt |
| 7921 | agcttaacgg tagtctttag atcataagaa atatataaat |
| | tagtatgcac cttatctgcc |
| 7981 | tgttgtgggt ttcttaaact tgcacttcct acccacccaa |
| | agatagatat cctttaaaga |
| 8041 | aaataaaggc agagaattaa aactggggag ccatttacta |
| | tgtcaccatc actgttaact |
| 8101 | gtttcccagc aatctaaact ttttgaagtt tcagaggtgt |
| | attttttttt tgtatatatg |
| 8161 | tctgtgtgat tgtattgttt tgtttctaaa tatacaagga |
| | attctttaaa tagagaaaaa |
| 8221 | ggttaatcct cactgaaaca ccaggatgcc cactggatat |
| | actaatctga acatctgtag |
| 8281 | gtagtttgtc atgaaaaagt ggagagaaga tgagactttt |
| | gaatgaatga aaaagggtat |
| 8341 | cttgataccc agaattcccc ccaaagtacg ggtaattcaa |
| | cctgcacagt tttctttcac |
| 8401 | tcaaagtgtt cagcacttgt gagtgaaaaa tcatgtaatt |
| | atctgtaaat atgtagctaa |
| 8461 | caaattgacc tagtttctgt atttttttgt ttttgtacta |
| | aagtttatag gtctgtgcca |
| 8521 | gctagagaga agttgctgtc attaccagtt gtggtcctag |
| | catctaaccc tgaaaccatc |
| 8581 | ctaggtgaca tttttagaat taatacttaa atgttaaaca |
| | gggggaaatg aagcttaatc |
| 8641 | atggtcaggt ttgagatctt ttgcagtgaa ataattttat |
| | ttaatataaa tgatcacatg |
| 8701 | tcctcaatca tgaatgaggt agggagcctc tctcccccag |
| | tggccatgtt tacaaaagtg |
| 8761 | tgttttgtct ataaagtgca agtgttttaa tgtttatgta |
| | aattatgcag gtgataacat |
| 8821 | ggtttggaac tgtttattgg gctctttaac tgaattttca |
| | aatgaaatga actatgctta |
| 8881 | ttgctggcac attgatccca tttctggaac atttttccta |
| | tttccagagt tacatatgtt |
| 8941 | cttttgtcat tacccaattt aacctccctt tctctgatat |
| | gccttgtagc caaagtatta |
| 9001 | aaggctgatg aacatagaca agggaaatgc atttcttaga |
| | aatccgtgaa ccctcagttg |
| 9061 | tatgctttca gtactcgtgt taatatgttt ctatggcaac |
| | tctgaggtca gtggtttaga |
| 9121 | aatgagatac cagtgttaat gaaaagtgtg tgctctttgc |
| | ttttgcatgg cttggcttag |
| 9181 | tatccaaggt atattagggc cacttgaaag catgaagacc |
| | agttatatag ggaacaggtt |
| 9241 | tctctcagtg gcacattttg ctttttctga gccccaaata |
| | cattgcctgg gcatgaacat |
| 9301 | tgttaccgta aattgcacat ggtcatggac tgaattatgt |
| | gactttaaag gatgtaactg |
| 9361 | cccaacattt gcagattctg ggtggtctat gtgaccattt |
| | gtctcgtatc caaaaacccc |
| 9421 | ggggctattg gaacccttcc aacacttttt cctttgtcat |
| | agacaagttt atatataact |
| 9481 | taccaagatg ttggctgtcc tggtgtattg ccagacagct |
| | ctcttttggt tcccattcca |
| 9541 | aatgtgctgc tgtccttctt tgcatttcac aatatcaaag |
| | aaaccaccac ccttcttcct |
| 9601 | aacagcattt tatgcctttt attccacatt aaatgggaat |
| | tgtgcctact taggagtgcc |
| 9661 | cctccaatta attacatgtg tccaagaata atccaagcta |
| | gagacacaag gtgggaaaac |
| 9721 | atttcaaaaa aaaaaagtcc tcttaaggcc agtaatttat |
| | ctgaaaaggt attttatcac |
| 9781 | accttgacac cttatatatg agcctattag gagctgcagg |
| | tggtttcata gggtaaaatc |
| 9841 | caagaaaaga gaaggatgtg tggggtttct attagaagat |
| | aattttgttc tcattttacc |
| 9901 | ttttctttta tgatccttct ctgctagaac aggttaattc |
| | tccaaatttg ttttgttttg |
| 9961 | ttttgttatt ttttagggaa ctcttttgca aaagcaatgg |
| | tcggatgtaa ataacattta |
| 10021 | aagtatagtg cacataactt ccccggactg ttccaatctg |
| | ataatttgta aatgctttag |
| 10081 | agttttttta attaacactt gtgttgctaa attctattta |
| | tgtaagtctg ctaaagtttt |
| 10141 | ttagcccact taaaacttaa gacaaccatt taaaataatg |
| | gatgggttac tatgagcaat |
| 10201 | ttcgctttca gaaccccctt gttttagtat atgaaaaagc |
| | ctaatgcgca ttaatgaggt |
| 10261 | tgaagagact atgagaaata tgtatagtgt atattttaaa |
| | acagctttgc ttgtattgtg |
| 10321 | aagatttaaa aacaaacttg agatttttaa cgtaactatt |
| | aacacagttt taacataagt |
| 10381 | tatcccactg ggtttaagag catcttgaat gtataatcct |
| | ttttgtaacc caggttggtt |
| 10441 | tctactttta ccagtcaccc aaacatattt atgtttttag |
| | ttttatgtac tcatttccct |
| 10501 | ttgttttcct caaacagcat gatttttttg cacatgtaga |
| | aattttttaa aagaaagaaa |
| 10561 | ttagtacatc attttctctg gattttcttc acttccctct |
| | tcctttctac taactccttc |
| 10621 | cttaaaggcc atatcactcc atttgcatta tttgtgcaaa |
| | tgccagggtt ggtttttatt |
| 10681 | tttatttttg ctatttacct aaaaaaagaa aatgcttcag |
| | tcaattgctt ttttatttaa |
| 10741 | aaaaaaaaag aaaaaaagct gtaaccttat catttctgag |
| | tagaccattg agcgatgaat |
| 10801 | gcacacctgt agtagcccag gaccagctgt ggtggctaaa |
| | gggaatatgt taattaagca |
| 10861 | agaggttctt ttctaaaagt ggtatctgtt atccacaatg |
| | tattttagtt attcccacaa |
| 10921 | gtcaggggtc cagataaaat gagggttatc agctaactga |
| | tatgctatca ttgaggttca |
| 10981 | tcaatgaatt tgtacatttc tagttccctt tggtgaaggg |
| | aaaaatgatg attttgcaag |
| 11041 | acctagattt tggcttggtt tcttgcctcc ttttttggca |
| | gccttcatct tctcatctcc |
| 11101 | caaaccccct gagcccgtag ggttttcata gtggacaaag |
| | aacttgtggt cttttaaaac |
| 11161 | tgggactgat acttttttga gagagtatcg tgtcgaaagt |
| | gtgatgttct accactttac |
| 11221 | caataactaa ttttaaatac acattgtcct ctcgattttt |
| | ggaccaaaca gacgctcaca |
| 11281 | gtggaggctt atcaagggtt gcattgggga agaagcctct |
| | ccctctctgt cagcaccagc |
| 11341 | tggtaaaggt gactgtacag atgtgcattt tccttttggt |
| | ataaatggtc cacagcacta |
| 11401 | actggtaagg cttattgtac agtatattgt cagtattctt |
| | ctggttcagc ataccttata |
| 11461 | gttcatatat aacctgtatt aattgtatag attgtgcatt |
| | aaaagctgtt accaagttgt |
| 11521 | cagaacataa gagcgaaaac aaggtcatat gtaatatttt |
| | gtttgtaagt atcctttgta |
| 11581 | tcatagcaaa ggaaatgttt aaaaaaatca actgtaataa |
| | agtaatttta gtacacaaaa |
| 11641 | aaaaaaaaaa aa |

An exemplary human PSG7 amino acid sequence is set forth below (SEQ ID NO: 196; GenBank Accession No: NP_002774.2, Version 2, incorporated herein by reference):

| | |
|---|---|
| 1 | mgplsappct ghitwkglll tasllnfwnp pttaqvtiea |
| | qppkvsegkd vlllvhnlpq |
| 61 | nltgyiwykg qirdlyhyvt syivdgqiik ygpaysgret |
| | vysnaslliq nvtqedtgsy |
| 121 | tlhiikrgdg tggvtgrftf tlyletpkps isssnfnpre |
| | ateaviltcd petpdasylw |
| 181 | wmngqslpmt hslqlsetnr tlylfgvtny tagpyeceir |
| | npvsasrsdp vtlnllpklp |
| 241 | kpyitinnln prenkdvstf tcepksenyt yiwwlngqsl |
| | pvsprvkrri enrililpsv |
| 301 | trnetgpyqc eirdryggir sdpvtlnvly gpdlpriyps |
| | ftyyhsgqnl ylscfadsnp |
| 361 | paqyswting kfqlsgqkls ipqittkhsg lyacsvrnsa |
| | tgkessksvt vrvsdwtlp |

An exemplary human PSG7 nucleic acid sequence is set forth below (SEQ ID NO: 197; GenBank Accession No: U18467.1, Version 1, incorporated herein by reference):

| | |
|---|---|
| 1 | attcgggcct aggctcatct ccacagagga gaacacgcag |
| | ggagcagaga ccatggggcc |
| 61 | cctctcagcc cctccctgca cacagcatat aacctggaaa |
| | gggctcctgc tcacagcatc |
| 121 | acttttaaac ttctggaacc cgcccaccac agcccaagtc |
| | acgattgaag cccagccacc |
| 181 | aaaagtttct gaggggaagg atgttcttct acttgtccac |
| | aatttgcccc agaatcttac |
| 241 | tggctacatc tggtacaaag gacaaatcag ggacctctac |
| | cattatgtta catcatatgt |
| 301 | agtagacggt caaataatta aatatgggcc tgcatacagt |
| | ggacgagaaa cagtatattc |
| 361 | caatgcatcc ctgctgatcc agaatgtcac ccaggaagac |
| | acaggatcct acactttaca |
| 421 | catcataaag cgaggtgatg ggactggagg agtaactgga |
| | cgtttcacct tcaccttata |
| 481 | cctggagact cccaaaccct ccatctccag cagcaatttc |
| | aaccccaggg aggccacgga |
| 541 | ggctgtgatc ttaacctgtg atcctgagac tccagatgca |
| | agctacctgt ggtggatgaa |
| 601 | tggtcagagc ctccctatga ctcacagctt gcagctgtct |
| | gaaaccaaca ggaccctcta |
| 661 | cctatttggt gtcacaaact atactgcagg accctatgaa |
| | tgtgaaatac ggaacccagt |
| 721 | gagtgccagc cgcagtgacc cagtcaccct gaatctcctc |
| | ccgaagctgc ccaagcccta |
| 781 | catcaccatc aataacttaa accccaggga gaataaggat |
| | gtctcaacct tcacctgtga |
| 841 | acctaagagt gagaactaca cctacatttg gtggctaaat |
| | ggtcagagcc tcccggtcag |
| 901 | tcccagggta aagcgacgca ttgaaaacag gatcctcatt |
| | ctacccagtg tcacgagaaa |
| 961 | tgaaacagga ccctatcaat gtgaaatacg ggaccgatat |
| | ggtggcatcc gcagtgaccc |
| 1021 | agtcaccctg aatgtcctct atggtccaga cctccccaga |
| | atttaccctt cgttcaccta |
| 1081 | ttaccattca ggacaaaacc tctacttgtc ctgctttgcg |
| | gactctaacc caccggcaca |
| 1141 | gtattcttgg acaattaatg ggaagtttca gctatcagga |
| | caaaagcttt ctatccccca |
| 1201 | gattactaca aagcatagcg ggctctatgc ttgctctgtt |
| | cgtaactcag ccactggcaa |
| 1261 | ggaaagctcc aaatccgtga cagtcagagt ctctgactgg |
| | acattaccct gaattctact |
| 1321 | agttcctcca attccatctt ctcccatgga acctcaaaga |
| | gcaagaccca ctctgttcca |
| 1381 | gaagccctat aagtcagagt tggacaactc aatgtaaatt |
| | tcatgggaaa atccttgtac |
| 1441 | ctgatgtctg agccactcag aactcaccaa aatgttcaac |
| | accataacaa cagctgctca |
| 1501 | aactgtaaac aaggaaaaca agttgatgac ttcacactgt |
| | ggacagtttt tcccaagatg |
| 1561 | tcagaataag actccccatc atgatgaggc tctcacccct |
| | cttagctgtc cttgcttgtg |
| 1621 | cctgcctctt tcacttggca ggataatgca gtcattagaa |
| | tttcacatgt agtataggag |
| 1681 | cttctgaggg taacaacaga gtgtcagata tgtcatctca |
| | acctcaaact tttacataac |
| 1741 | atctcaggag gaaatgtggc tctctccatc ttgcatacag |
| | ggctcccaat agaaatgaac |
| 1801 | acagagatat tgcctgtgtg tttgcagaga agatggtttc |
| | tataaagagt aggaaagctg |
| 1861 | aaattatagt agagtcccct ttaaatgcac attgtgtgga |
| | tggctctcac catttcctaa |
| 1921 | gagatacatt gtaaaacgtg acagtaagac tgattctagc |
| | agaataaaac atgtactaca |
| 1981 | tttgctaaa |

An exemplary human PSG8 amino acid sequence is set forth below (SEQ ID NO: 198; GenBank Accession No: AAI37501.1, Version 1, incorporated herein by reference):

| | |
|---|---|
| 1 | mgllsappct qritwkglll tasllnfwnp pttaqvtiea |
| | qptkvsegkd vlllvhnlpq |
| 61 | nltgyiwykg qirdlyhyit syvvdgqiii ygpaysgret |
| | iysnaslliq nvtqedagsy |
| 121 | tlhiimggde nrgvtghftf tlyletpkps isssklnpre |
| | ameavsltcd petpdasylw |
| 181 | wmngqslpms hrlqlsetnr tlfllgvtky tagpyeceir |
| | npvsasrsdp ftlnllpklp |
| 241 | kpyitinnlk prenkdvlnf tcepksenyt yiwwlngqsl |
| | pvsprvkrpi enrililpsv |
| 301 | trnetgpyqc eirdqyggir sypvtlnvly gpdlpriyps |
| | ftyyrsgevl ylscsadsnp |
| 361 | paqyswting kfqlsgqklf ipqittkhsg lyacsvrnsa |
| | tgkessksmt vkvsgkripv |
| 421 | slaigi |

An exemplary human PSG8 nucleic acid sequence is set forth below (SEQ ID NO: 199; GenBank Accession No: BC142628.1, Version 1, incorporated herein by reference):

| | |
|---|---|
| 1 | ggacagcact gctgagagct gtgctcagga agcttctgga |
| | tcctaggctc atctccacag |
| 61 | aggagaacac acagacagca gagaccatgg ggctcctctc |
| | agcccctccc tgcacacagc |
| 121 | gcatcacctg gaaggggctc ctgctcacag catcactttt |
| | aaacttctgg aacccaccca |
| 181 | cgactgccca agtcacgatt gaagcccagc caaccaaagt |
| | ttctgagggg aaggatgttc |
| 241 | ttctacttgt ccacaatttg ccccagaatc ttactggcta |
| | catctggtac aaagggcaaa |
| 301 | tcagggacct ctaccattac attacatcat atgtagtaga |
| | cggtcaaata attatatatg |
| 361 | ggcctgcata cagtggacga gaaacaatat attccaatgc |
| | atccctgctg atccagaatg |
| 421 | tcacccagga agacgcagga tcctacacct tacacatcat |
| | aatgggaggt gatgagaata |
| 481 | gaggagtaac tggacatttc accttcacct tatatctgga |
| | gactcccaag ccctccatct |
| 541 | ccagcagcaa attaaacccc agggaggcca tggaggctgt |
| | gagcttaacc tgtgatcctg |
| 601 | agactccgga cgcaagctac ctgtggtgga tgaatggtca |
| | gagcctccct atgtctcaca |
| 661 | ggttgcagtt gtctgaaacc aacaggaccc tctttctatt |
| | gggtgtcaca aagtacactg |
| 721 | caggacccta tgaatgtgaa atacggaacc cagtgagtgc |
| | cagccgcagt gacccattca |
| 781 | ccctgaatct cctcccgaag ctgcccaagc cctacatcac |
| | catcaacaac ttaaaaccca |
| 841 | gggagaataa ggatgtctta aacttcacct gtgaacctaa |
| | gagtgagaac tacacctaca |
| 901 | tttggtggct aaatggtcag agcctcccgg tcagtcccag |
| | ggtaaagcga cccattgaaa |
| 961 | acaggatcct cattctaccc agtgtcacga gaaatgaaac |
| | aggaccctat caatgtgaaa |
| 1021 | taagggacca atatggtggc atccgcagtt acccagtcac |
| | cctgaatgtc ctctatggtc |
| 1081 | cagacctccc cagaatttac ccttcattca cctattaccg |
| | ttcaggagaa gtcctctact |
| 1141 | tgtcctgttc tgcggactct aacccaccgg cacagtattc |
| | ttggacaatt aatgggaagt |
| 1201 | ttcagctatc aggacaaaag ctctttatcc cccaaattac |
| | tacaaagcat agcgggctct |
| 1261 | atgcttgctc tgttcgtaac tcagccactg gcaaggaaag |
| | ctccaaatcc atgacagtaa |
| 1321 | aagtctctga ctggacatta ccctgaattc tactagttcc |
| | tccaattcca ttttcttcca |
| 1381 | tggaatcgct aagaaaaaga cccactctgt tccagaagcc |
| | ctataagctg gaggtggaca |
| 1441 | actcaatgta aatttcatgg gaaaaccctt gtacctgaag |
| | cgtgagccac tcagaactca |
| 1501 | ctaaaatgtt cgacaccata acaacagatg ctcaaactgt |
| | aaaccaggac aacaagtgga |
| 1561 | tgacttcaca ctgtggacag tttttcccaa gatgtcagaa |
| | caagactccc catcatgatg |
| 1621 | aggctctcac ccctcttaac tgtccttgct catgcctgcc |
| | tctttcactt ggcaggataa |
| 1681 | tgcagtcatt agaatttcac atgtagtagc ttctgagggt |
| | aacaatagag tgtcagatat |
| 1741 | gtcatctcaa cccaaacttt tacataacat ctcaggggga |
| | aatgtggctc tctccacctt |
| 1801 | gcatacagga ctcccaatag aaatgaacac agagatattg |
| | cccgtgtgtt tgcagataag |
| 1861 | atggtttcta tgaagaggta ggaaagctga aattataata |
| | gagtcccctt taaatgcaca |
| 1921 | ttctgtggat ggctctcgcc atttcctaag agatacattg |
| | taaaatgtga cagtaatact |
| 1981 | gattctagca gaataaaaca tgtaccacat ttgctaatac |
| | aaaaaaaaaa aaaaaaaaaa |
| 2041 | aaaaaaaaaa aaaaaaaaaa aaaaaaaaaa aaaaaaaaaa |
| | aaaaaaaaaa aaaaaaaaaa |
| 2101 | aaaaaaaaaa aaaaaaaaaa aaaaaaaaaa aaaaaaaaaa |
| | aaaa |

An exemplary human PSG9 amino acid sequence is set forth below (SEQ ID NO: 200; GenBank Accession No: AAH20759.1, Version 1, incorporated herein by reference):

| | |
|---|---|
| 1 | mgplpapsct qritwkglll tasllnfwnp pttaevtiea |
| | qppkvsegkd vlllvhnlpq |
| 61 | nlpgyfwykg emtdlyhyii syivdgkiii ygpaysgret |
| | vysnaslliq nvtrkdagty |
| 121 | tlhiikrgde treeirhftf tlyletpkpy isssnlnpre |
| | ameavrlicd petldasylw |
| 181 | wmngqslpvt hrlqlsktnr tlylfgvtky iagpyeceir |
| | npvsasrsdp vtlnllpklp |
| 241 | ipyitinnln prenkdvlaf tcepksenyt yiwwlngqsl |
| | pvspgvkrpi enrililpsv |
| 301 | trnetgpyqc eiqdrygglr snpvilnvly gpdlpriyps |
| | ftyyrsgenl dlscftesnp |
| 361 | paeyfwting kfqqsgqklf ipqitrnhsg lyacsvhnsa |
| | tgkeisksmt vkvsgpchgd |
| 421 | ltesqs |

An exemplary human PSG9 nucleic acid sequence is set forth below (SEQ ID NO: 201; GenBank Accession No: BC020759.1, Version 1, incorporated herein by reference):

| | |
|---|---|
| 1 | agaaggagga aggacagcac agctgacagc cgtgctcaga |
| | cagcttctgg atcccaggct |
| 61 | catctccaca gaggagaaca cacaggcagc agagaccatg |
| | gggcccctcc cagccccttc |
| 121 | ctgcacacag cgcatcacct ggaaggggct cctgctcaca |
| | gcatcacttt taaacttctg |
| 181 | gaacccgccc accactgccg aagtcacgat tgaagcccag |
| | ccacccaaag tttctgaggg |
| 241 | gaaggatgtt cttctacttg tccacaattt gccccagaat |
| | cttcctggct acttctggta |
| 301 | caaaggggaa atgacggacc tctaccatta cattatatcg |
| | tatatagttg atggtaaaat |
| 361 | aattatatat gggcctgcat acagtggaag agaaacagta |
| | tattccaacg catccctgct |
| 421 | gatccagaat gtcacccgga aggatgcagg aacctacacc |
| | ttacacatca taaagcgagg |
| 481 | tgatgagact agagaagaaa ttcgacattt caccttcacc |
| | ttatacttgg agactcccaa |
| 541 | gccctacatc tccagcagca acttaaaccc cagggaggcc |
| | atggaggctg tgcgcttaat |
| 601 | ctgtgatcct gagactctgg acgcaagcta cctatggtgg |
| | atgaatggtc agagcctccc |
| 661 | tgtgactcac aggttgcagc tgtccaaaac caacaggacc |
| | ctctatctat ttggtgtcac |
| 721 | aaagtatatt gcaggaccct atgaatgtga aatacggaac |
| | ccagtgagtg ccagtcgcag |
| 781 | tgacccagtc accctgaatc tcctcccgaa gctgcccatc |
| | ccctacatca ccatcaacaa |
| 841 | cttaaacccc agggagaata aggatgtctt agccttcacc |
| | tgtgaaccta agagtgagaa |
| 901 | ctacacctac atttggtggc taaacggtca gagcctcccc |
| | gtcagtcccg gggtaaagcg |
| 961 | acccattgaa aacaggatac tcattctacc cagtgtcacg |
| | agaaatgaaa caggacccta |
| 1021 | tcaatgtgaa atacaggacc gatatggtgg cctccgcagt |
| | aacccagtca tcctaaatgt |
| 1081 | cctctatggt ccagacctcc ccagaattta cccttcattc |
| | acctattacc gttcaggaga |
| 1141 | aaacctcgac ttgtcctgct tcacggaatc taacccaccg |
| | gcagagtatt tttggacaat |
| 1201 | taatgggaag tttcagcaat caggacaaaa gctctttatc |
| | ccccaaatta ctagaaatca |
| 1261 | tagcgggctc tatgcttgct ctgttcataa ctcagccact |
| | ggcaaggaaa tctccaaatc |
| 1321 | catgacagtc aaagtctctg gtccctgcca tggagacctg |
| | acagagtctc agtcatgact |
| 1381 | gcaacaactg agacactgag aaaaagaaca ggctgatacc |
| | ttcatgaaat tcaagacaaa |
| 1441 | gaagaaaaaa actcaatgtt attggactaa ataatcaaaa |
| | ggataatgtt ttcataattt |
| 1501 | tttattggaa aatgtgctga ttctttgaat gttttattct |
| | ccagatttat gaactttttt |
| 1561 | tcttcagcaa ttggtaaagt atacttttat aaacaaaaat |
| | tgaaatattt gcttttgctg |
| 1621 | tctatctgaa tgccccagaa ttgtgaaact attcatgagt |
| | attcataggt ttatggtaat |
| 1681 | aaagttattt gcacatgttc caaaaaaaaa aaaaaaaaaa |
| | aaaaaaaaaa a |

An exemplary human PSG11 amino acid sequence is set forth below (SEQ ID NO: 202 GenBank Accession No: AAA60203.1, Version 1, incorporated herein by reference):

| | |
|---|---|
| 1 | mgpfpapsct qritwkglll tasllnfwnp pttaevtiea |
| | qppkvsegkd vlllvhnlpq |
| 61 | nlpgyfwykg emtdlyhyii syivdgkiii ygpaysgret |
| | vysnaslliq nvtrkdagty |
| 121 | tlhiikrgde treeirhftf tlyletpkpy isssnlnpre |
| | ameavrlicd petldasylw |
| 181 | wmngqslpvt hrlqlsktnr tlylfgvtky iagpyeceir |
| | npvsairsdp vtlnllpklp |
| 241 | ipyitinnln prenkdvlaf tcepksenyt yiwwlngqsl |
| | pvspgvkrpi enrililpsv |
| 301 | trnetgpyqc eirdrygglr snpvilnvly gpdlpriyps |
| | ftyyrsgenl dlscftesnp |
| 361 | paeyfwting kfqqsgqklf ipqitrnhsg lyacsvhnsa |
| | tgkeisksmt vkvsgpchgd |
| 421 | ltesqs |

An exemplary human PSG11 nucleic acid sequence is set forth below (SEQ ID NO: 203; GenBank Accession No: M58591.1, Version 1, incorporated herein by reference):

| | |
|---|---|
| 1 | cagccgtgct cagacagctt ctggatccta ggctcatctc |
| | cacagaggag aacacgcagg |
| 61 | cagcagagac catggggccc ttcccagccc cttcctgcac |
| | acagcgcatc acctggaagg |
| 121 | ggctcctgct cacagcatca cttttaaact tctggaaccc |
| | gcccaccact gccgaagtca |
| 181 | cgattgaagc ccagccaccc aaagtttctg aggggaagga |
| | tgttcttcta cttgtccaca |
| 241 | atttgcccca gaatcttcct ggctacttct ggtacaaagg |
| | ggaaatgacg gacctctacc |
| 301 | attacattat atcgtatata gttgatggta aaataattat |
| | atatgggcct gcatacagtg |
| 361 | gaagagaaac agtatattcc aacgcatccc tgctgatcca |
| | gaatgtcacc cggaaggatg |
| 421 | caggaaccta caccttacac atcataaagc gaggtgatga |
| | gactagagaa gaaattcgac |
| 481 | atttcacctt caccttatac ttggagactc ccaagcccta |
| | catctccagc agcaacttaa |
| 541 | accccaggga ggccatggag gctgtgcgct taatctgtga |
| | tcctgagact ctggacgcaa |
| 601 | gctacctatg gtggatgaat ggtcagagcc tccctgtgac |
| | tcacaggttg cagctgtcca |
| 661 | aaaccaacag gaccctctat ctatttggtg tcacaaagta |
| | tattgcagga ccctatgaat |
| 721 | gtgaaatacg gaacccagtg agtgccattc gcagtgaccc |
| | agtcaccctg aatctcctcc |
| 781 | cgaagctgcc catcccctac atcaccatca acaacttaaa |
| | ccccagggag aataaggatg |
| 841 | tcttagcctt cacctgtgaa cctaagagtg agaactacac |
| | ctacatttgg tggctaaacg |
| 901 | gtcagagcct ccccgtcagt cccggggtaa agcgacccat |
| | tgaaaacagg atactcattc |
| 961 | tacccagtgt cacgagaaat gaaacaggac cctatcaatg |
| | tgaaatacgg gaccgatatg |
| 1021 | gtggcctccg cagtaaccca gtcatcctaa atgtcctcta |
| | tggtccagac ctccccagaa |
| 1081 | tttacccttc attcacctat taccgttcag gagaaaacct |
| | cgacttgtcc tgcttcacgg |
| 1141 | aatctaaccc accggcagag tatttttgga caattaatgg |
| | gaagtttcag caatcaggac |
| 1201 | aaaagctctt tatcccccaa attactagaa atcatagcgg |
| | gctctatgct tgctctgttc |
| 1261 | ataactcagc cactggcaag gaaatctcca aatccatgac |
| | agtcaaagtc tctggtccct |
| 1321 | gccatggaga cctgacagag tctcagtcat gactgcaaca |
| | actgagacac tgagaaaaag |
| 1381 | aacaggctga taccttcatg aaattcaaga caaagaagaa |
| | aaaaactcaa tgttattgga |
| 1441 | ctaaataatc aaaaggataa tgttttcata attttttatt |
| | ggaaaatgtg ctgattcttt |
| 1501 | gaatgtttta ttctccagat ttatgaactt tttttcttca |
| | gcaattggta aagtatactt |
| 1561 | ttgtaaacaa aaattgaaat atttgctttt gctgtctatc |
| | tgaatgcccc agaattgtga |
| 1621 | aactattcat gagtattcat aggtttatgg taataaagtt |
| | atttgcacat gttccgta |

An exemplary human miR-185 nucleic acid sequence is set forth below (SEQ ID NO: 204; GenBank Accession No: NR_029706.1, Version 1, incorporated herein by reference):

| | |
|---|---|
| 1 | agggggcgag ggattggaga gaaaggcagt tcctgatggt |
| | cccctcccca ggggctggct |
| 61 | ttcctctggt ccttccctcc ca |

An exemplary human miR-513a2 nucleic acid sequence is set forth below (SEQ ID NO: 205; GenBank Accession No: LM609506.1, Version 1, incorporated herein by reference):

| | |
|---|---|
| 1 | ggatgccaca ttcagccatt cagtgtgcag tgcctttcac |
| | agggaggtgt catttatgtg |
| 61 | aactaaaata taaatttcac ctttctgaga agggtaatgt |
| | acagcatgca ctgcatatgt |
| 121 | ggtgtcc |

An exemplary human HMGB1 amino acid sequence is set forth below (SEQ ID NO: 210 GenBank Accession No: CAG33144.1, Version 1, incorporated herein by reference):

| | |
|---|---|
| 1 | mgkgdpkkpr gkmssyaffv qtcreehkkk hpdasvnfse |
| | fskkcserwk tmsakekgkf |
| 61 | edmakadkar yeremktyip pkgetkkkfk dpnapkrpps |
| | afflfcseyr pkikgehpgl |
| 121 | sigdvakklg emwnntaadd kqpyekkaak lkekyekdia |
| | ayrakgkpda akkgvvkaek |
| 181 | skkkkeeeed eedeedeeee edeededeee ddddd |

An exemplary human HMGB1 nucleic acid sequence is set forth below (SEQ ID NO: 211; GenBank Accession No: NM_001313893.1, Version 1, incorporated herein by reference):

| | |
|---|---|
| 1 | tttctgcgga gggattacgc tgacgaaaga gacctgcttg |
| | cgcgtcgctg ttccgtggtc |
| 61 | cgcgcgagcg tggtcgggag ccgctggttc ctggggtgac |
| | ccgcggaggt gggagaggga |
| 121 | agggcttccg aagccggcgg gggtgccatg gaccctctcc |
| | gccggcgcgg ccttcacagc |
| 181 | tgggccgcgc cgggcatccg tagtccgctc tcccaaagcc |
| | tcggtggagc tgaagctgcc |
| 241 | acagagtgca tgttcacaaa gggtcatcac acacggagct |
| | gcccctccct gtctccctag |
| 301 | agcccatctt cgaggccagg ggcttttcta ccaggattct |
| | ggggtgtttc tcctcctttc |
| 361 | ctccctccca gatcttctca cggtaagggg agcagcgaaa |
| | gcgcagggac tttgcattcc |
| 421 | acgacccgtt ctgactagtc aacagccgat ctgtccctgc |
| | tgctctaatt ccagctgccc |
| 481 | tgccttgttt taacttcaga gaaaggggga gttctcattt |
| | gataagttta agcctttgct |
| 541 | ttcgtaggaa ggtcatgtgg cttaagggac atcgtgaccg |
| | cgtatgctat ttctgcctgt |
| 601 | gcattctaaa tcttgggggc agcatattcc agaagtcctt |
| | ttggtcgatt ggttctgtgt |
| 661 | cctaggataa caatttgtag tttctgacca tttctttaca |
| | gaaaaaccac aattggtatt |
| 721 | ttggactgcg gggtttttta gtggtctcaa actaaatgat |
| | tatattctgg aataatgctg |
| 781 | acaatttgga tagggtggtg tggaggaaac aagtctcgtg |
| | tagaagaaat tatttagtaa |
| 841 | aaaggatttt agtttttggt acttctgaat gcaaatggcc |
| | aaggaatcca gcagtttgtt |
| 901 | ggggtttctc cagacaaaaa taggctgaaa aataactaaa |
| | catgggcaaa ggagatccta |
| 961 | agaagccgag aggcaaaatg tcatcatatg cattttttgt |
| | gcaaacttgt cgggaggagc |
| 1021 | ataagaagaa gcacccagat gcttcagtca acttctcaga |
| | gttttctaag aagtgctcag |
| 1081 | agaggtggaa gaccatgtct gctaaagaga aaggaaaatt |
| | tgaagatatg gcaaaagcgg |
| 1141 | acaaggcccg ttatgaaaga gaaatgaaaa cctatatccc |
| | tcccaaaggg gagacaaaaa |
| 1201 | agaagttcaa ggatcccaat gcacccaaga ggcctccttc |
| | ggccttcttc ctcttctgct |
| 1261 | ctgagtatcg cccaaaaatc aaaggagaac atcctggcct |
| | gtccattggt gatgttgcga |
| 1321 | agaaactggg agagatgtgg aataacactg ctgcagatga |
| | caagcagcct tatgaaaaga |
| 1381 | aggctgcgaa gctgaaggaa aaatacgaaa aggatattgc |
| | tgcatatcga gctaaaggaa |
| 1441 | agcctgatgc agcaaaaaag ggagttgtca aggctgaaaa |
| | aagcaagaaa aagaaggaag |
| 1501 | aggaggaaga tgaggaagat gaagaggatg aggaggagga |
| | ggaagatgaa gaagatgaag |
| 1561 | atgaagaaga agatgatgat gatgaataag ttggttctag |
| | cgcagttttt tttttcttgt |
| 1621 | ctataaagca tttaaccccc ctgtacacaa ctcactcctt |
| | ttaaagaaaa aaattgaaat |
| 1681 | gtaaggctgt gtaagatttg tttttaaact gtacagtgtc |
| | tttttttgta tagttaacac |
| 1741 | actaccgaat gtgtctttag atagccctgt cctggtggta |
| | ttttcaatag ccactaacct |
| 1801 | tgcctggtac agtatggggg ttgtaaattg gcatggaaat |
| | ttaaagcagg ttcttgttgg |
| 1861 | tgcacagcac aaattagtta tatatgggga tggtagtttt |
| | ttcatcttca gttgtctctg |
| 1921 | atgcagctta tacgaaataa ttgttgttct gttaactgaa |
| | taccactctg taattgcaaa |
| 1981 | aaaaaaaaaa aagttgcagc tgttttgttg acattctgaa |
| | tgcttctaag taaatacaat |
| 2041 | tttttttatt agtattgttg tccttttcat aggtctgaaa |
| | tttttcttct tgaggggaag |
| 2101 | ctagtctttt gcttttgccc attttgaatc acatgaatta |
| | ttacagtgtt tatcctttca |
| 2161 | tatagttagc taataaaaag cttttgtcta cacaccctgc |
| | atatcataat gggggtaaag |
| 2221 | ttaagttgag atagttttca tccataactg aacatccaaa |
| | atcttgatca gttaagaaat |
| 2281 | ttcacatagc ccacttacat ttacaaactg aagagtaatc |
| | aatctactca aagcatggga |
| 2341 | ttattagaat caaacatttt gaaagtctgt ccttgaagga |
| | ctaatagaaa agtatgttct |
| 2401 | aacctttaca tgaggactct attctttaac tcccattacc |
| | atgtaatggc agttatattt |
| 2461 | tgcagttccc acattaaaga agacctgaga atgtatcccc |
| | aaaagcgtga gcttaaaata |
| 2521 | caagactgcc atattaaatt ttttgttgac attagtctca |
| | gtgaagacta tgaaaatgct |
| 2581 | ggctatagat gtcttttccc atttatctaa atatggactg |
| | ctcaggaaac gagactttcc |
| 2641 | attacaagta tttttaatta attgggccag cttttcaaac |
| | aaagatgcca cattcaaaat |
| 2701 | agggtatatt ttcctatatt acggtttgcc cctttataaa |
| | tccaagtaga taggaagaaa |
| 2761 | gaagacaaac tttgcatctc agtatgaatt attcaattta |
| | tttgaatgat ttttctttac |
| 2821 | aaaacaaact cattcattag tcatgtttat ctgcttagga |
| | gtttagggaa caatttggca |
| 2881 | attttgtggt tttcgagatt atcgttttct taaagtgcca |
| | gtattttaaa atagcgttct |
| 2941 | tgtaatttta cacgcttttg tgatggagtg ctgttttgtt |
| | atataattta gacttggatt |
| 3001 | ctttccattt gcatttgttt atgtaatttc aggaggaata |
| | ctgaacatct gagtcctgga |
| 3061 | tgatactaat aaactaataa ttgcagaggt tttaaatact |
| | agttaaatgg ctttcactta |
| 3121 | agaacttaag attttgttac atatttttaa atcttgtttc |
| | taataatacc tcttagcagt |
| 3181 | accttttaaa taagtataag ggatggcaaa gtttttccct |
| | ttaaaaatac tcactttatg |
| 3241 | cttataaata ggttaatggg ctgataaaag gttttgtcaa |
| | acattgcaag tattcggtgc |
| 3301 | tatatataaa ggaggaaaaa ctagttttac tttcagaatg |
| | atttaaacaa gatttttaaa |
| 3361 | aacaagatac atgcaagcga acagcagggt tagtgatagg |
| | ctgcaattgt gtcgaacatc |
| 3421 | agattttttg ttaagaggag caaatgactc aatctgattt |
| | agatggaagt ttctactgta |
| 3481 | tagaaatcac cattaatcac caacattaat aattctgatc |
| | catttaaaat gaattctggc |
| 3541 | tcaaggagaa tttgtaactt tagtaggtac gtcatgacaa |
| | ctaccatttt tttaagatgt |
| 3601 | tgagaatggg aacagttttt ttagggttta ttcttgacca |
| | cagatcttaa gaaaatggac |
| 3661 | aaaacccctc ttcaatctga agattagtat ggtttggtgt |
| | tctaacagta tcccctagaa |
| 3721 | gttggatgtc taaaactcaa gtaaatggaa gtgggaggca |
| | atttagataa gtgtaaagcc |
| 3781 | ttgtaactga agatgatttt ttttagaaag tgtatagaaa |
| | ctattttaat gccaagatag |
| 3841 | ttacagtgct gtggggttta aagactttgt tgacatcaag |
| | aaaagactaa atctataatt |
| 3901 | aattgggcca acttttaaaa tgaagatgct ttttaaaact |
| | aatgaactaa gatgtataaa |
| 3961 | tcttagtttt tttgtatttt aaagataggc atatggcata |
| | ttgattaacg agtcaaattt |
| 4021 | cctaactttg ctgtgcaaag gttgagagct attgctgatt |
| | agttaccaca gttctgatga |
| 4081 | tcgtcccatc acagtgttgt taatgtttgc tgtatttatt |
| | aattttctta aagtgaaatc |
| 4141 | tgaaaaatga aatttgtgtg tcctgtgtac ccgaggggta |
| | atgattaaat gataaagata |
| 4201 | agaaaagcgc ccatgtaaca caaactgcca ttcaacaggt |
| | atttccctta ctacctaagg |
| 4261 | aattgtaacc attgctcaga cattgtagga tttaactatg |
| | ttgaaaacta caggagaggc |
| 4321 | cgggcgcagt ggctcacgcc tgtaatccca gcactttggg |
| | aggccaaggc gggcagatca |
| 4381 | cgaggtcagg agattgagac catcctggct aacgtggtga |
| | aaccccgcct ctactaaaaa |
| 4441 | tacaaaaaat tagccaagcg tggtgctggg cgcctgtagt |
| | cccagtaact caggaggctg |
| 4501 | aggcaggaga atggcgtgaa cccgggaggc ggaggttgca |
| | gtgagccgag attgtgccac |
| 4561 | tgcactccag cctgggtgac agagcaagac tccatctcaa |
| | aaaaaaaaaa aaaacacagg |
| 4621 | agagacaact ggtttttgaa tgaaatacat gggtactgcc |
| | ttgcttgaca tcacatagtc |
| 4681 | cttgatgaaa gttcacattt aggtctgctt ggtacaatac |
| | gcctcctaaa aaggtccttg |
| 4741 | atgaaagttc acatttaggt ctgcttggta caacacgcct |
| | cctgaaaggg tctgatagct |
| 4801 | ttcagtagca gtaagacact tgcatgtgat ggtaaggtat |
| | ctgcaaattt gcacacaccg |
| 4861 | tacacagctt aagtcttaga attaacttgc taaaatgtga |
| | gcctttggta attaggctgt |
| 4921 | tttattaggg agtgtgataa tatttgaatt tcttttcata |
| | tttgtgcttt gtgtcatttt |
| 4981 | caaatgaccc ttgaaatgta ttttaaaagt agataaaagc |
| | cagaaagtga |

### Pharmaceutical Therapeutics

For therapeutic uses, the compositions or agents described herein may be administered systemically, for example, formulated in a pharmaceutically-acceptable buffer such as physiological saline. Preferable routes of administration include, for example, subcutaneous, intravenous, interperitoneally, intramuscular, or intradermal injections that provide continuous, sustained levels of the drug in the patient. Treatment of human patients or other animals will be carried out using a therapeutically effective amount of a therapeutic identified herein in a physiologically-acceptable carrier. Suitable carriers and their formulation are described, for example, in Remington's Pharmaceutical Sciences by E. W. Martin. The amount of the therapeutic agent to be administered varies depending upon the manner of administration, the age and body weight of the patient, and with the clinical symptoms of the neoplasia, i.e., the melanoma. Generally, amounts will be in the range of those used for other agents used in the treatment of other diseases associated with neoplasia, although in certain instances lower amounts will be needed because of the increased specificity of the compound. For example, a therapeutic compound is administered at a dosage that is cytotoxic to a neoplastic cell.

### Formulation of Pharmaceutical Compositions

The administration of a compound or a combination of compounds for the treatment of a neoplasia, e.g., a melanoma, may be by any suitable means that results in a concentration of the therapeutic that, combined with other components, is effective in ameliorating, reducing, or stabilizing a neoplasia. The compound may be contained in any appropriate amount in any suitable carrier substance, and is generally present in an amount of 1-95% by weight of the total weight of the composition. The composition may be provided in a dosage form that is suitable for parenteral (e.g., subcutaneously, intravenously, intramuscularly, or intraperitoneally) administration route. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Human dosage amounts can initially be determined by extrapolating from the amount of compound used in mice, as a skilled artisan recognizes it is routine in the art to modify the dosage for humans compared to animal models. In certain embodiments it is envisioned that the dosage may vary from between about 1 μg compound/Kg body weight to about 5000 mg compound/Kg body weight; or from about 5 mg/Kg body weight to about 4000 mg/Kg body weight or from about 10 mg/Kg body weight to about 3000 mg/Kg body weight; or from about 50 mg/Kg body weight to about 2000 mg/Kg body weight; or from about 100 mg/Kg body weight to about 1000 mg/Kg body weight; or from about 150 mg/Kg body weight to about 500 mg/Kg body weight. In other cases, this dose may be about 1, 5, 10, 25, 50, 75, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1600, 1700, 1800, 1900, 2000, 2500, 3000, 3500, 4000, 4500, or 5000 mg/Kg body weight. In other aspects, it is envisaged that doses may be in the range of about 5 mg compound/Kg body to about 20 mg compound/Kg body. In other embodiments, the doses may be about 8, 10, 12, 14, 16 or 18 mg/Kg body weight. Of course, this dosage amount may be adjusted upward or downward, as is routinely done in such treatment protocols, depending on the results of the initial clinical trials and the needs of a particular patient.

Pharmaceutical compositions according to the invention may be formulated to release the active compound substantially immediately upon administration or at any predetermined time or time period after administration. The latter types of compositions are generally known as controlled release formulations, which include (i) formulations that create a substantially constant concentration of the drug within the body over an extended period of time; (ii) formulations that after a predetermined lag time create a substantially constant concentration of the drug within the body over an extended period of time; (iii) formulations that sustain action during a predetermined time period by maintaining a relatively, constant, effective level in the body with concomitant minimization of undesirable side effects associated with fluctuations in the plasma level of the active substance (sawtooth kinetic pattern); (iv) formulations that localize action by, e.g., spatial placement of a controlled release composition adjacent to or in contact with the thymus; (v) formulations that allow for convenient dosing, such that doses are administered, for example, once every one or two weeks; and (vi) formulations that target a neoplasia by using carriers or chemical derivatives to deliver the therapeutic agent to a particular cell type (e.g., neoplastic cell). For some applications, controlled release formulations obviate the need for frequent dosing during the day to sustain the plasma level at a therapeutic level.

Any of a number of strategies can be pursued in order to obtain controlled release in which the rate of release outweighs the rate of metabolism of the compound in question. In one example, controlled release is obtained by appropriate selection of various formulation parameters and ingredients, including, e.g., various types of controlled release compositions and coatings. Thus, the therapeutic is formulated with appropriate excipients into a pharmaceutical composition that, upon administration, releases the therapeutic in a controlled manner. Examples include single or multiple unit tablet or capsule compositions, oil solutions, suspensions, emulsions, microcapsules, microspheres, molecular complexes, nanoparticles, patches, and liposomes.

### Parenteral Compositions

The pharmaceutical composition may be administered parenterally by injection, infusion or implantation (subcutaneous, intravenous, intramuscular, intraperitoneal, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation. Formulations can be found in Remington: The Science and Practice of Pharmacy, supra.

Compositions for parenteral use may be provided in unit dosage forms (e.g., in single-dose ampoules), or in vials containing several doses and in which a suitable preservative may be added (see below). The composition may be in the form of a solution, a suspension, an emulsion, an infusion device, or a delivery device for implantation, or it may be presented as a dry powder to be reconstituted with water or another suitable vehicle before use. Apart from the active agent that reduces or ameliorates a neoplasia, the composition may include suitable parenterally acceptable carriers and/or excipients. The active therapeutic agent(s) may be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing, agents.

As indicated above, the pharmaceutical compositions according to the invention may be in the form suitable for sterile injection. To prepare such a composition, the suitable active antineoplastic therapeutic(s) are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution and dextrose solution. The aqueous formulation may also contain one or more preservatives (e.g., methyl, ethyl or n-propyl p-hydroxybenzoate). In cases where one of the compounds is only sparingly or slightly soluble in water, a dissolution enhancing or solubilizing agent can be added, or the solvent may include 10-60% w/w of propylene glycol.

### Controlled Release Parenteral Compositions

Controlled release parenteral compositions may be in form of aqueous suspensions, microspheres, microcapsules, magnetic microspheres, oil solutions, oil suspensions, or emulsions. Alternatively, the active drug may be incorporated in biocompatible carriers, liposomes, nanoparticles, implants, or infusion devices.

Materials for use in the preparation of microspheres and/or microcapsules are, e.g., biodegradable/bioerodible polymers such as polygalactin, poly-(isobutyl cyanoacrylate), poly(2-hydroxyethyl-L-glutam-nine) and, poly(lactic acid). Biocompatible carriers that may be used when formulating a controlled release parenteral formulation are carbohydrates (e.g., dextrans), proteins (e.g., albumin), lipoproteins, or antibodies. Materials for use in implants can be non-biodegradable (e.g., polydimethyl siloxane) or biodegradable (e.g., poly(caprolactone), poly(lactic acid), poly(glycolic acid) or poly(ortho esters) or combinations thereof).

### Kits or Pharmaceutical Systems

The present compositions may be assembled into kits or pharmaceutical systems for use in ameliorating a neoplasia (e.g., melanoma). Kits or pharmaceutical systems according to this aspect of the invention comprise a carrier means, such as a box, carton, tube or the like, having in close confinement therein one or more container means, such as vials, tubes, ampoules, or bottles. The kits or pharmaceutical systems of the invention may also comprise associated instructions for using the agents of the invention.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### Examples

### Example 1: Molecular Signatures of Resistance (No Benefit)

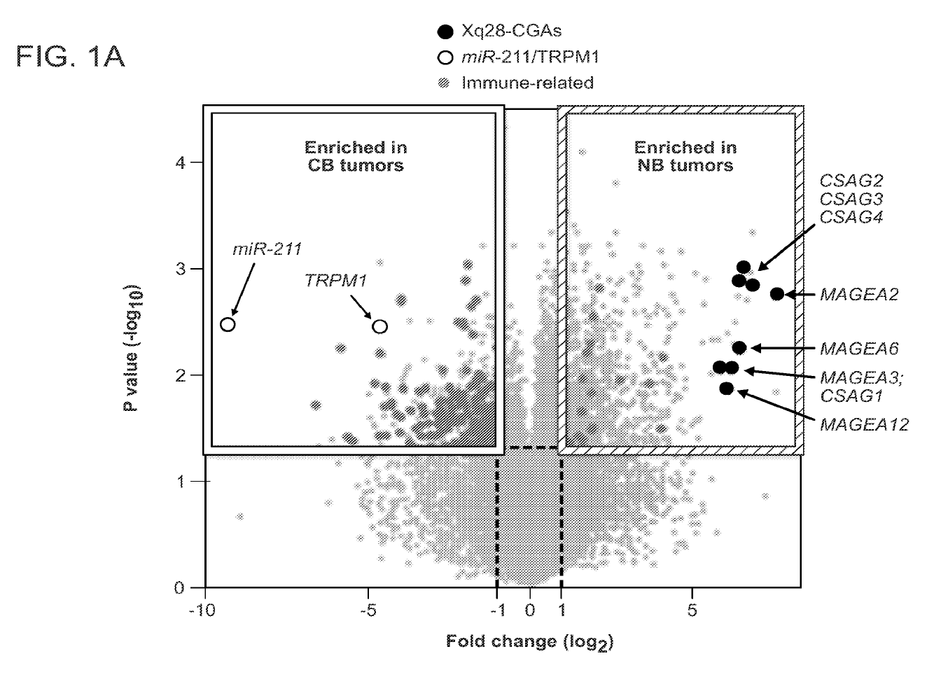

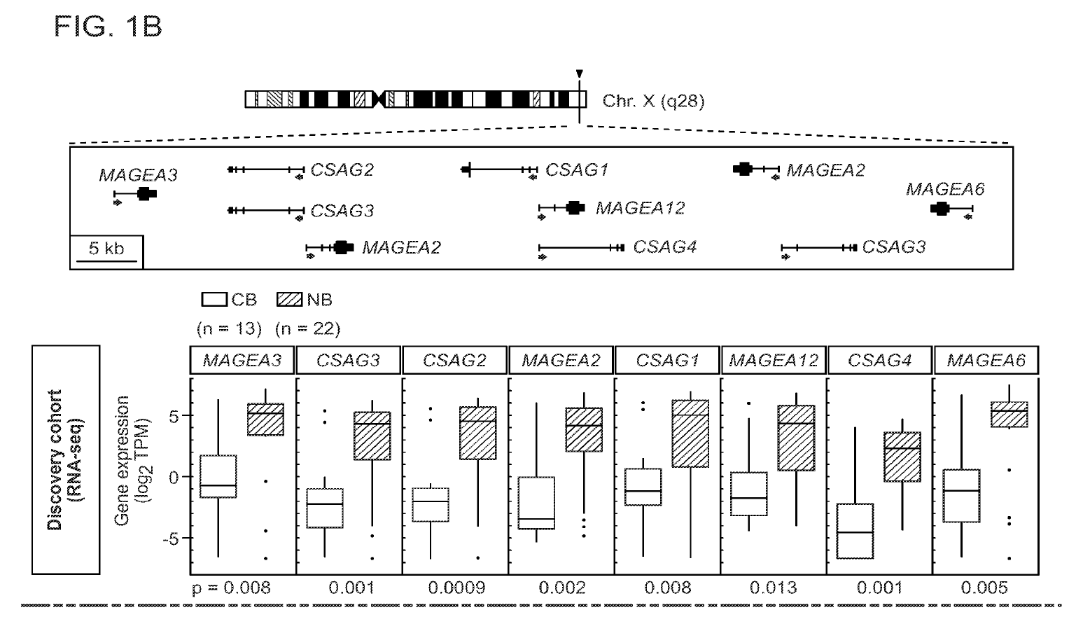

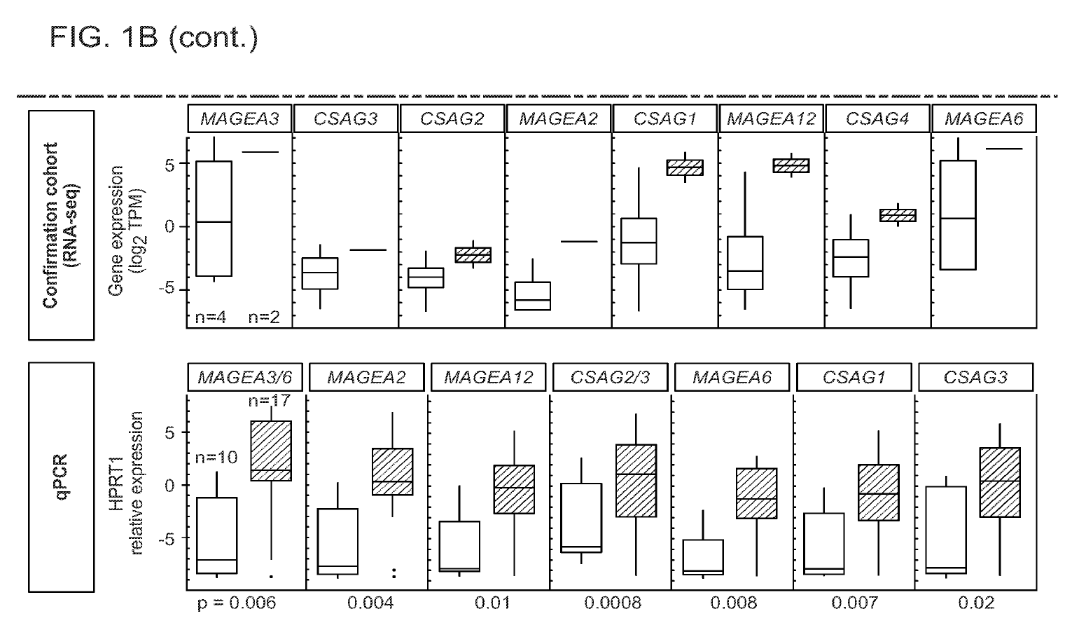

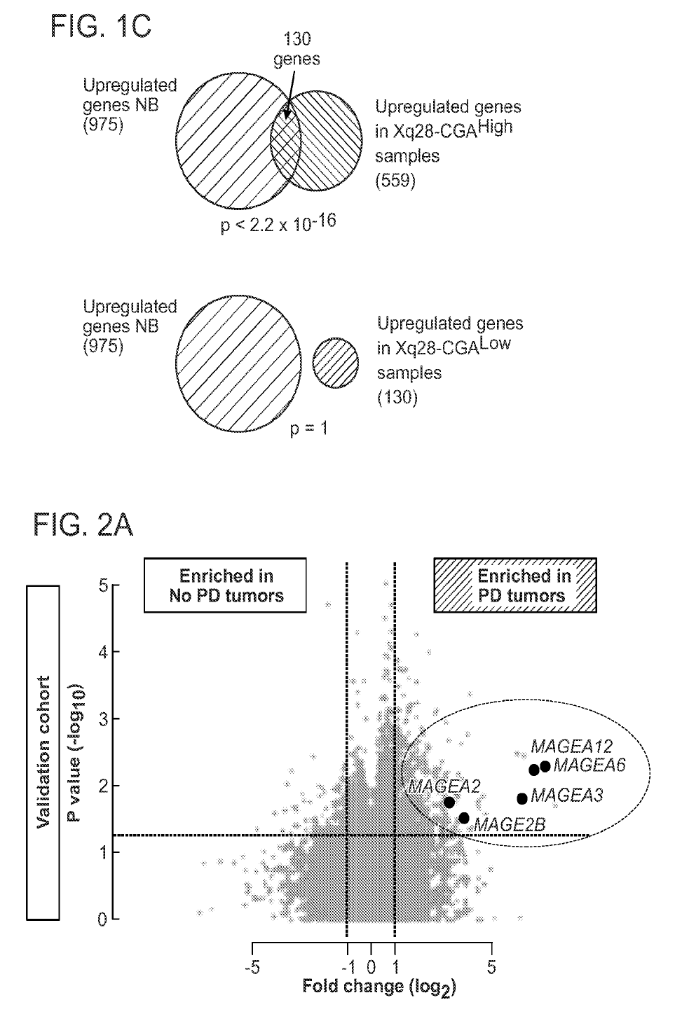

First, expression data from a previously published cohort of pre-ipilimumab treated melanoma patients (Cohort 1) was analyzed (Van Allen et al., 2015 Science, 350(6257):207-11, incorporated herein by reference). The patients were classified into three groups based on their post-treatment outcome: (i) patients who achieved complete or partial response by Response Evaluation Criteria in Solid Tumors (RECIST) criteria or stable disease by RECIST criteria with overall survival greater than one year (“clinical benefit/CB;” n=13); (ii) patients who had progressive disease by RECIST criteria or stable disease with overall survival less than 1 year (“no benefit/NB;” n=22); and (iii) patients showing early progression on ipilimumab (progression-free survival (PFS)<6 months) with overall survival >2 years (“long-term survival with no clinical benefit/LTS;” n=5). The LTS group was removed from the differential expression analysis for three reasons: 1) their unique clinical course suggests a potentially different clinicobiology; 2) the small number of samples precludes any meaningful comparisons with the other two groups; and 3) the focus was purely on expression patterns distinguishing clear benefit from clear resistance. Therefore, an unbiased differential gene expression analysis was performed between the CB and NB groups. Because of the limited number of patients, the statistical stringency was relaxed in the unbiased differential expression analysis by forgoing multiple hypothesis correction. Using a cutoff of 2-fold difference between the absolute medians of the two groups (unadjusted Mann-Whitney p<0.05), 975 genes co-enriched in the ‘no benefit’ tumors (FIG. 1A) were identified.

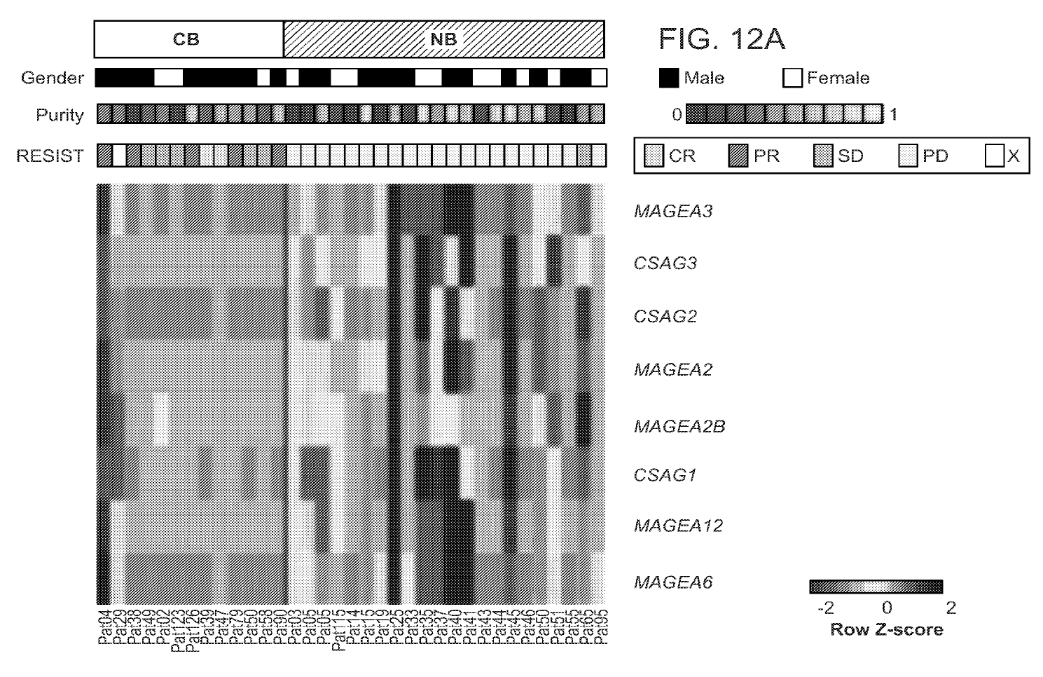

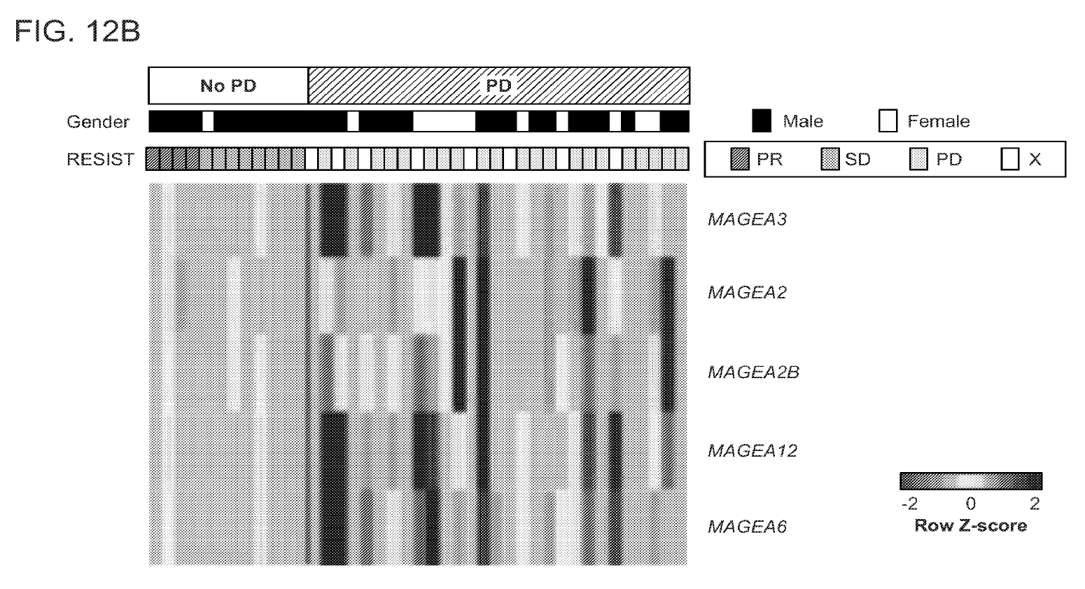

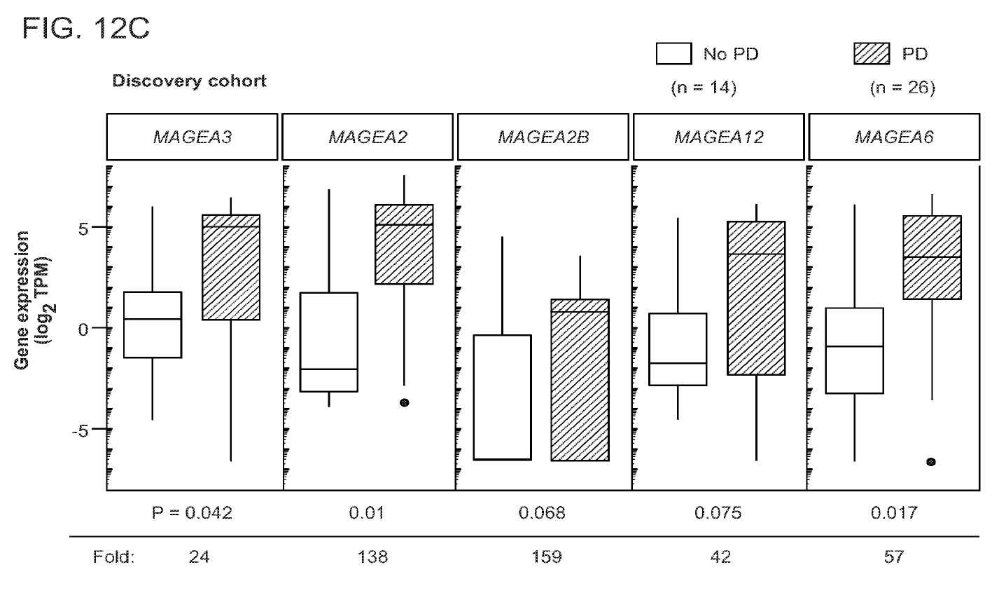

Strikingly, 8 of the top 10 genes enriching 60-180 fold in ‘NB’ tumors clustered within a narrow 75 Kb region of chromosome Xq28 (FIG. 1A, FIG. 1B, FIG. 12A), representing 0.0023% of the human genome. All 8 genes (MAGEA3, MAGEA6, CSAG1, MAGEA12, MAGEA2, CSAG2, and CSAG3 and CSAG4) encoded cancer-germline antigens (CGAs), a large family of 140 members notable for their restricted expression in testis and placenta but re-expression across many tumor types. While many subclusters of CGA's are located on Xq28, this particular subcluster has demonstrated an expression pattern independent of surrounding CGA clusters; in fact, the synchronous expression of these 8 clustered genes may be driven by their unique inverted repeat DNA structure. 14/22 NB tumors showed upregulation of at least one of these 8 CGA's versus 2/13 CB tumors (p=0.0125).

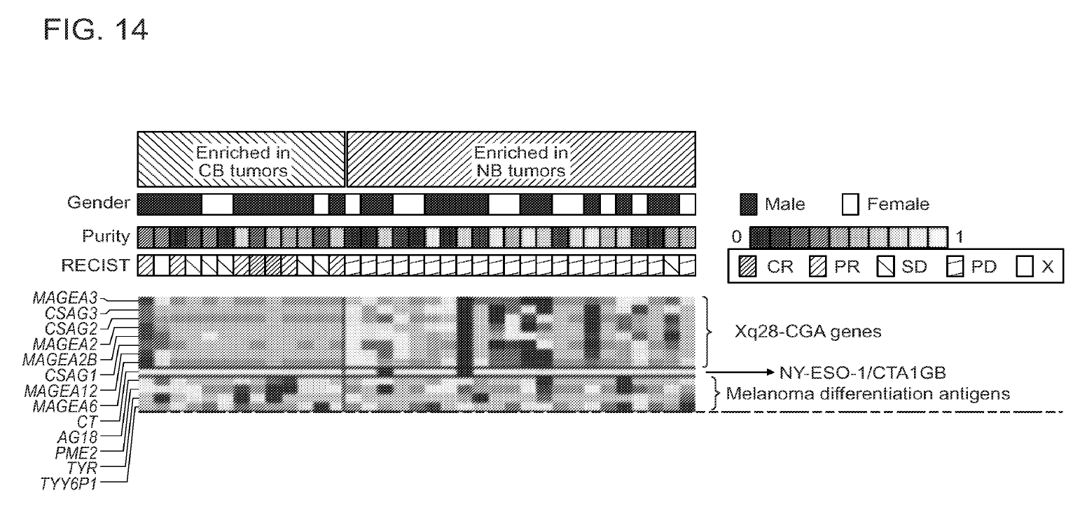

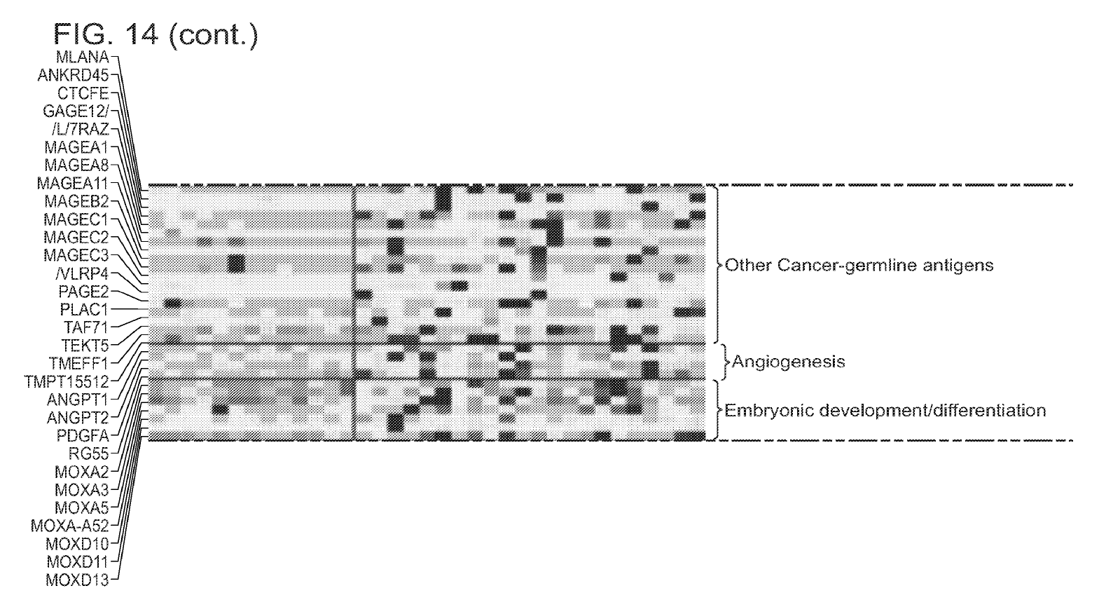

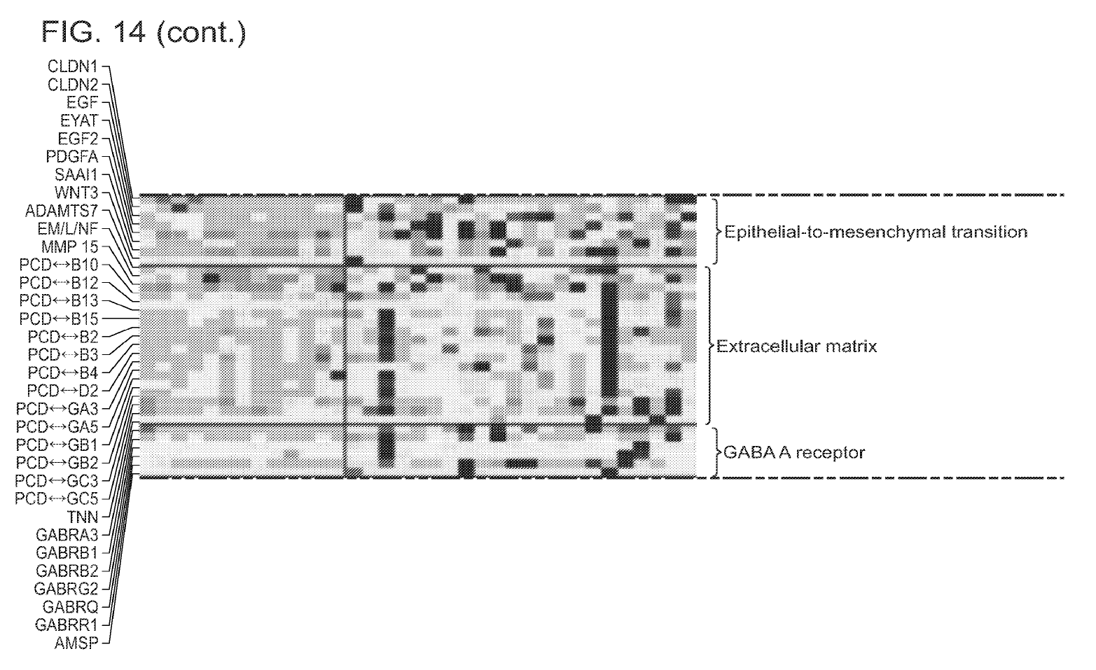

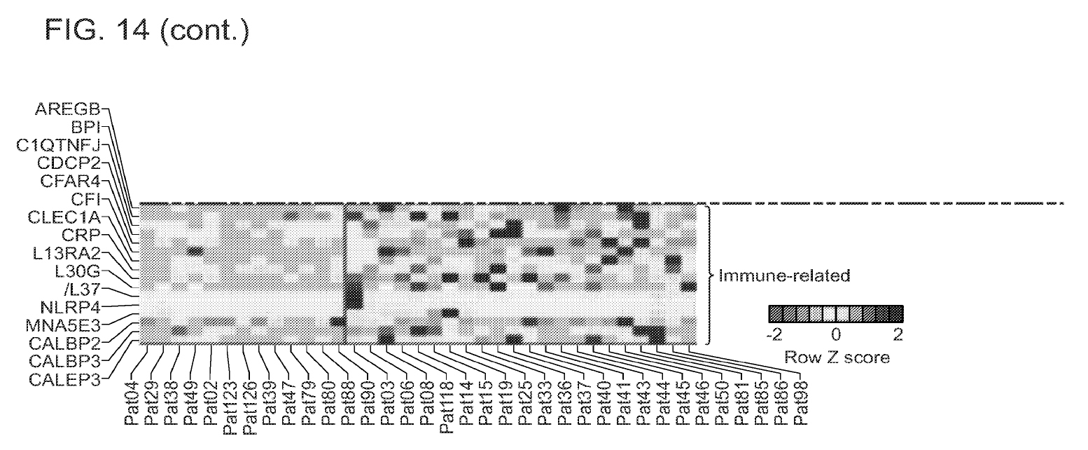

In addition to the CGA cluster on Xq28, increased expression of additional CGAs was identified in NB samples, though none were as highly expressed as those at the CRMA locus (Table 2, FIG. 14). Moreover, previously described melanoma antigens, such as NY-ESO-1, whose humoral and cellular responses have been linked to clinical outcome (Yuan et al., 2011), or differentiation antigens (e.g. TYR, TYRP1, PMEL, and MLANA), were not differentially expressed (FIG. 14). Multiple genes involved in immune suppression enriched in ‘no benefit’ tumors, in particular another family of embryonically-restricted genes known as the pregnancy-specific glycoproteins (PSG1, PSG2, PSG4, PSG5, PSG6, PSG7, PSG8, PSG9, and PSG11; 7-48 fold). Secreted by the syncytiotrophoblasts of the placenta during pregnancy, the PSGs impair T cell proliferation and secrete the immunosuppressive cytokines IL-10 and TGF-B1, presumably to drive a locally immunosuppressive microenvironment required for fetal tolerance. Moreover, multiple subunits of the GABA A receptor, which has been implicated in mediating suppression of inflammatory macrophages and anti-tumor T cells, were also enriched in nonresponding tumors (GABRA3, GABRB1, GABRB2, GABRG2, GABRQ, and GABRR1; 2-108 fold). In contrast, no components of the GABA B receptor were similarly enriched, consistent with its lack of demonstrative immunosuppressive activity.

Other differentially expressed genes corresponding to anti-CTLA4 resistance included epithelial-to-mesenchymal transition (CLDN1, CLDN2, EYA1, SNAI1, TGFB2, WNT3), embryonic development/differentiation (HOXD13, HOXD11, HOXA2, HOXA5, HOXD10), angiogenesis (ANGPT1, ANG2, PDGFA), and extracellular matrix (PCDHB2, PCDHB3, PCDHB6, PCDHB10, PCDHGA3, PCDHGB1, PCDHGB2, EMILIN1, TNN). Genes listed in Table 2.

Next, another previously published clinical cohort consisting of chemotherapy-naïve responding (n=4) and resistant (n=2) pre-anti-CTLA4 melanomas (confirmation cohort) was queried (Snyder et al., 2014 The New England Journal of Medicine, 371(23):2189-99, incorporated herein by reference). Here too, CGA's from the Xq28 locus were upregulated in resistant tumors (FIG. 1B), confirming previous findings.

To further validate these results in a larger cohort, the transcriptomes of 473 metastatic melanomas from The Cancer Genome Atlas (TCGA) were analyzed, dividing them into “CGA-Xq28 high” and “CGA-Xq28 low” groups based on their expression of these 8 CGA's (Cancer Genome Atlas N. Genomic Classification of Cutaneous Melanoma. 2015 Cell, 161(7):1681-96, incorporated herein by reference). A statistically significant overlap was identified between genes co-enriched with the CTA-Xq28 locus in TCGA and genes enriched with nonresponding pre-anti-CTLA4 tumors (p<10⁻¹⁶) (TABLE 3, 4). As expected, no overlap was seen between “CGA-Xq28 high”-associated genes in TCGA and genes associated with responding tumors (p=1) (FIG. 1C).

Similarly, significant overlaps were identified between “CGA-Xq28 low”-associated genes in TCGA and genes associated with clinical benefit (p=1.6×10⁻⁵) but no overlaps with genes associated with “no benefit” tumors (p=1). Thus the enrichment of these 8 CTA's from the Xq28 locus in primary resistant melanomas to anti-CTLA4 therapy is consistently observed among three different cohorts.

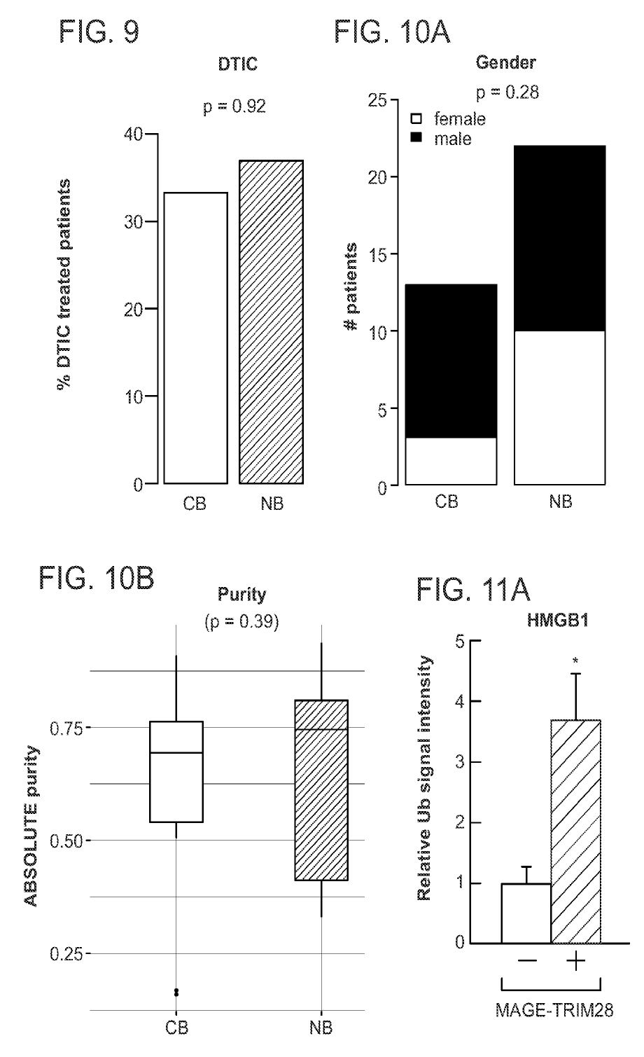

The cohort was heavily pretreated with dacarbazine (DTIC) or temozolamide, two epigenetic modifiers. However, no effect of history of DTIC/temozolamide treatment on clinical outcome was identified (FIG. 9).

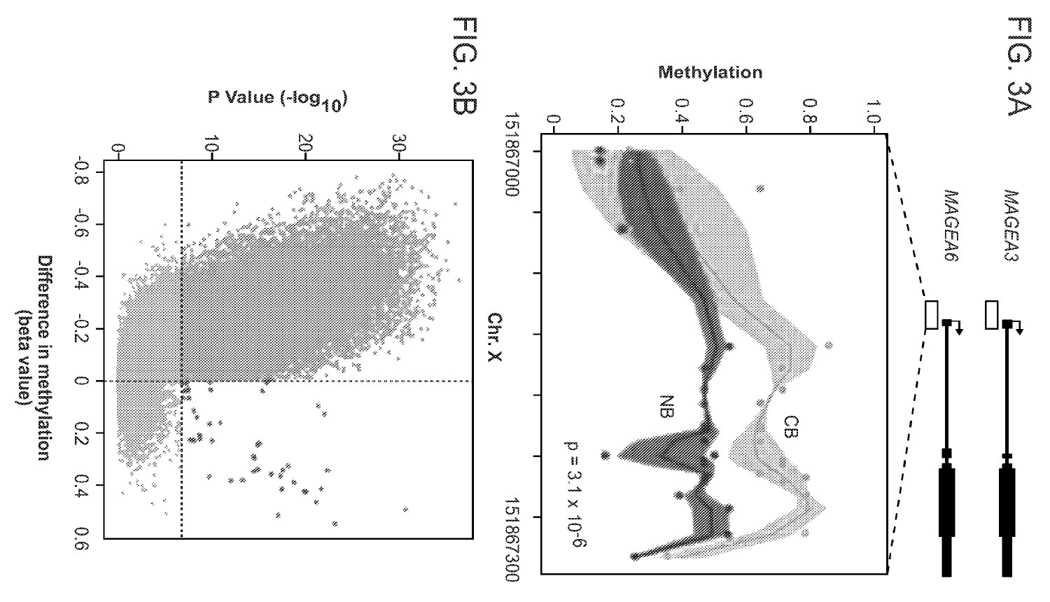

Because CGA expression is known to be regulated by DNA methylation (Sigalotti et al., 2002 Journal of immunotherapy, 25:16-26; Fratta et al., 2011 Molecular oncology, 5:164-82), locus specific methylation analysis was performed for the promoters of MAGEA3 and MAGEA6 as well as unique methylation sites within the gene bodies of MAGEA3, MAGEA6, and MAGE12. Within NB samples, significantly decreased DNA methylation was observed throughout the promoters for the MAGEA3 and MAGEA6 genes (FIG. 3A; n=3 CB vs n=3 NB, p=3×10⁻⁶) suggesting that methylation status of the Xq28-CGA locus is associated with clinical outcome to ipilimumab.

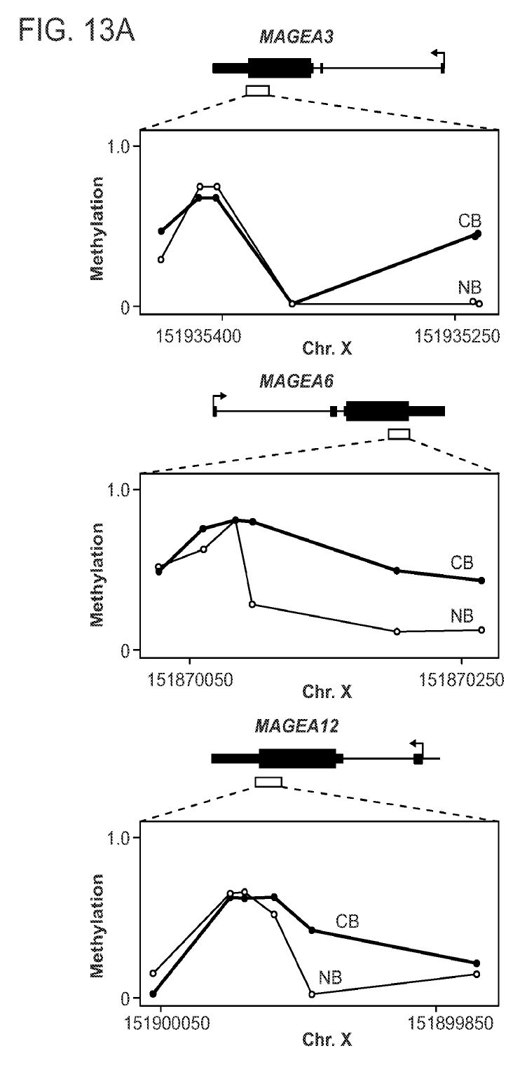

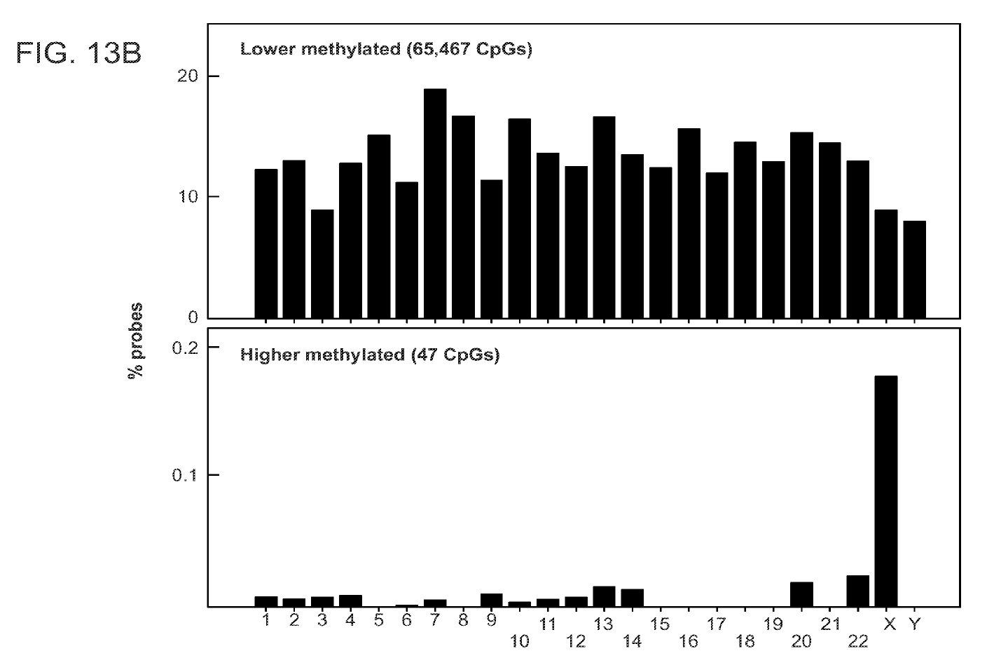

To further investigate DNA methylation patterns associated with CRMA expression, methylation data from TCGA melanoma samples was queried. Differential methylation analysis of 485,577 probes between samples with high and low expression of the CRMA locus (see STAR Methods for details) revealed 47 probes relatively hypermethylated in the “CRMA-high” group compared to 65,467 in the “CRMA-low” group (FIG. 3B). These 65,467 probes mapped across the genome, suggesting global hypomethylation in melanoma samples with high CRMA expression (FIG. 13B). Furthermore, methylation-specific PCR of MAGE-A2, MAGE-A3, and MAGE-A12 show decreased methylation in non-responding patients (FIG. 13A). Methylation status of the CRMA locus may therefore also be associated with clinical outcome to ipilimumab.

### Example 2: Molecular Signatures of Response (Clinical Benefit)

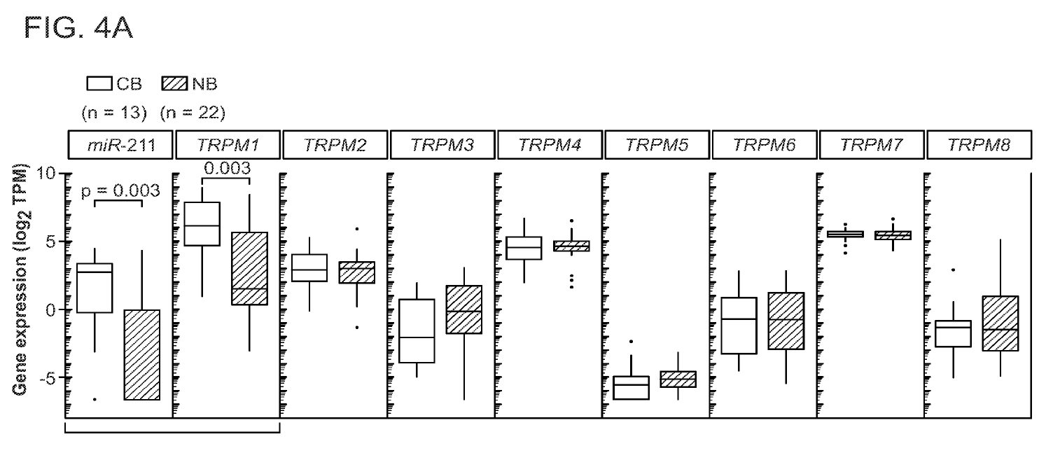

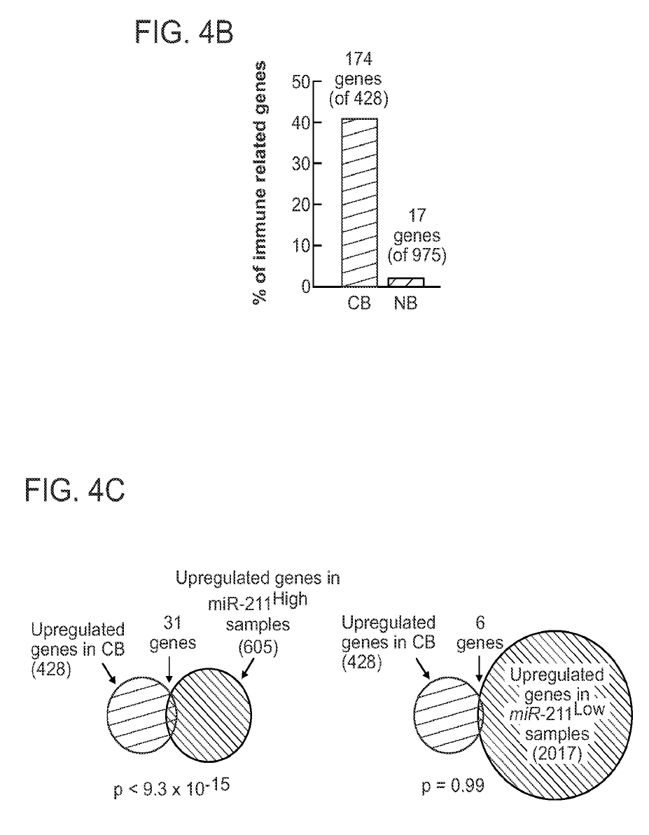

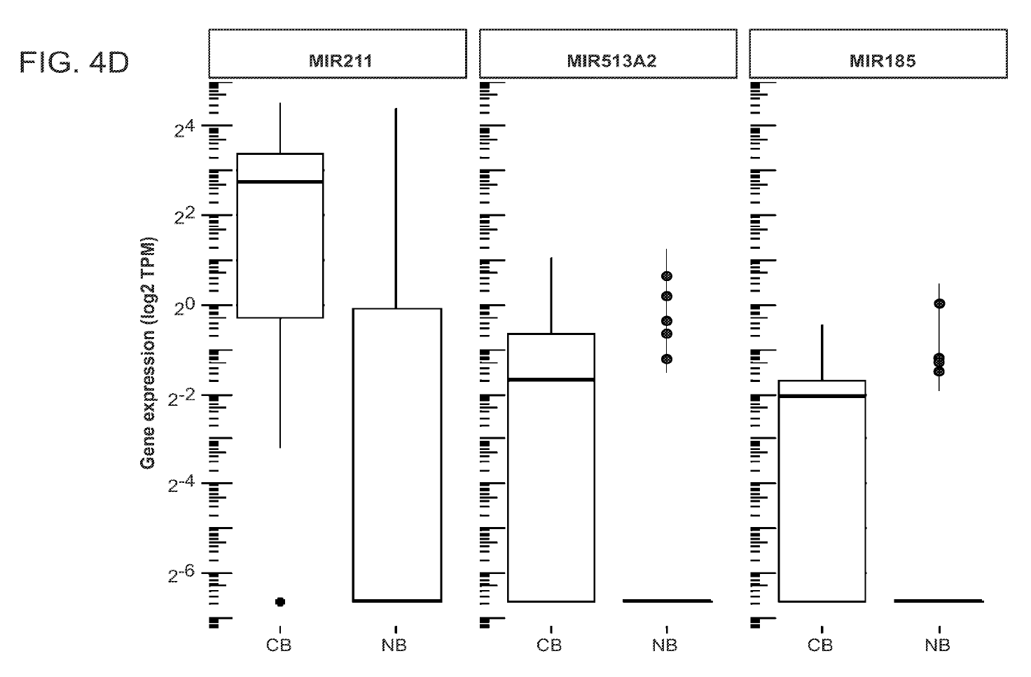

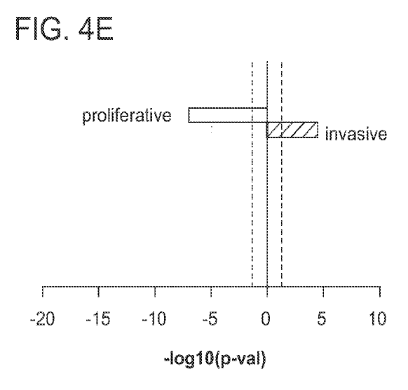

Using a cutoff of 2-fold difference between the absolute medians of the two groups (nominal Mann-Whitney p<0.05), 175 protein-coding genes and 8 RNA genes co-enriched in the ‘clinical benefit’ tumors were identified. As the most upregulated gene, microRNA-211 (miR-211) was detected over 700-fold compared to ‘no benefit’ tumors (FIG. 1A, 4A). Housed within the melastatin gene (TRPM1, which was also upregulated over 30-fold), miR-211 has been recently discovered to account for the well-described tumor suppressive activity of melastatin by reducing melanoma invasive activity through effects on multiple processes including the TGF-beta signaling pathway. Indeed, among all 8 melastatin family members, only TRPM1 (FIG. 4A) was significantly enriched in ‘clinical benefit’ tumors. To evaluate whether miR-211 served a prognostic and/or a predictive role in the cohort, miR-211 levels were compared only between patients with a complete or partial response versus progressive disease. All patients with stable disease were removed from the analysis. Notably, it was observed that miR-211 levels remained significantly upregulated in the complete response/partial response group versus the progressive disease group, suggesting its ability to predict ipilimumab response in addition to prognosticating a more indolent clinical course. We also found miR-185 and miR-513a2 to be upregulated 24 and 31-fold respectively.

In contrast to the immunosuppressive nature of genes associated with primary resistance, an inflammatory, activated immunologic response was identified in the tumor microenvironment of ‘clinical benefit’ tumors, consistent with previous findings (FIG. 1B, 4B). Of 428 genes enriched in responding tumors, 174 (60%) were identified as immune-related. In contrast, in nonresponding tumors, only 17 of 975 (3%) protein-coding and RNA-associated genes were immune-related (FIG. 4B, 11C).

These immune-related genes were classified as involved in T cell infiltration (CD2, CD6, CXCL13), receptor signaling (CD3D, CD3E, CD3G, LCK, and T cell receptor alpha and beta genes [n=19]), activation (CD28, ICOS, EOMES, IL2RB, FASLG, SLAMF6), and cytotoxicity (GNLY, GZMA, GZMB, GZMH, GZMK, PRF1). Interestingly, an enrichment of immature T cells was noted from increased expression of the pre-T cell receptor alpha chain (PTCRA). Additionally, a striking number of immunoglobulin heavy and light chain genes (n=33) were upregulated in ‘clinical benefit’ tumors, thereby implicating humoral immunity (CD19, CD72, FCRL1/3, MS4A1). Dysfunction of a diverse immune infiltrate was suggested by enrichment of immune inhibitory receptors specific to or preferentially expressed by T cells (CTLA4, LAG3), B cells (CTLA4, FCRL1, FCRL3), macrophages (CD5L) and eosinophils/mast cells (SIGLEC8), depicting a paralyzed anti-tumor immune infiltrate. Also, upregulation of FAIM3/TOSO, the Fc receptor for IgM that is expressed on B and T cells, was observed. Recently, single cell transcriptomic studies implicated both FAIM3 and CD5L as key regulators of Th17 pathogenicity.

To validate the association of miR-211 with clinical benefit, TCGA melanoma transcriptomes were queried. It was identified that genes co-enriched with miR-211 significantly overlapped with genes enriched in clinically benefiting tumors (p=2.5×10⁻¹³) whereas no significant overlap was seen between genes co-enriched with miR-211 and genes enriched in resistant tumors (p=0.99) (FIG. 4C). Investigating the 22 genes that co-enriched with both response in the clinical cohort and miR-211 expression in TCGA, CD5L, IL12RB2, FAIM3, and PTCRA emerged, confirming the association of anti-CTLA4 response, miR-211 expression and diverse immune subpopulations. As described herein, miR-185 and miR-513A2 were significantly upregulated in clinically benefiting tumors (FIG. 4D). Furthermore, because all three miRs induce a proliferative melanoma phenotype while suppressing the invasive phenotype, the enrichment of proliferative and invasive gene signatures with clinical outcomes were investigated. It was identified that proliferative signatures significantly enrich in clinically benefiting tumors, while invasive signatures significantly enrich in no benefit tumors (FIG. 4E).

### Example 3: Molecular Signatures of Clinical Outcome to CTLA4 Blockade are Unique and can Discriminate Response and Resistance

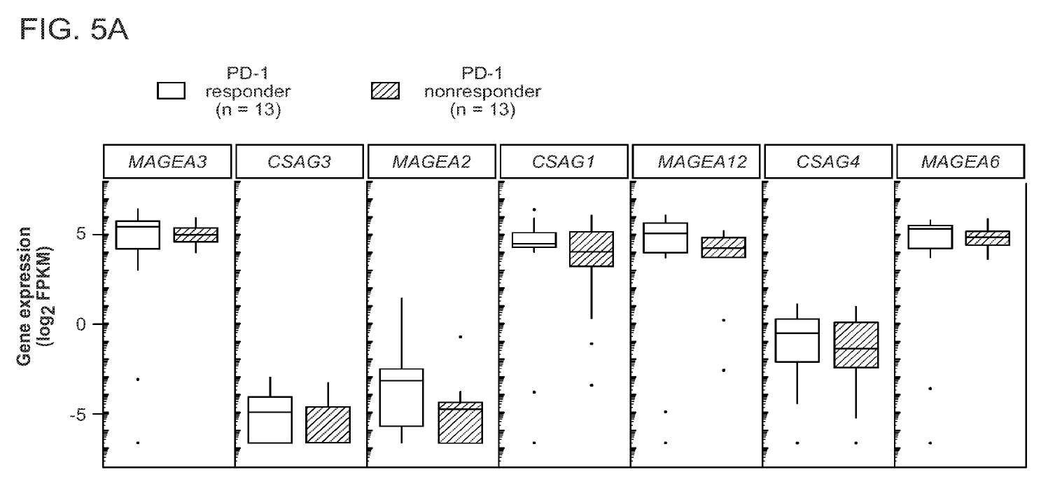

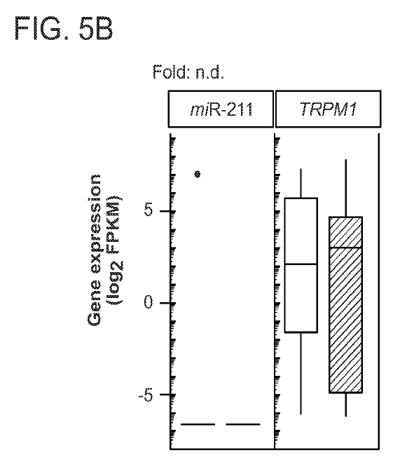

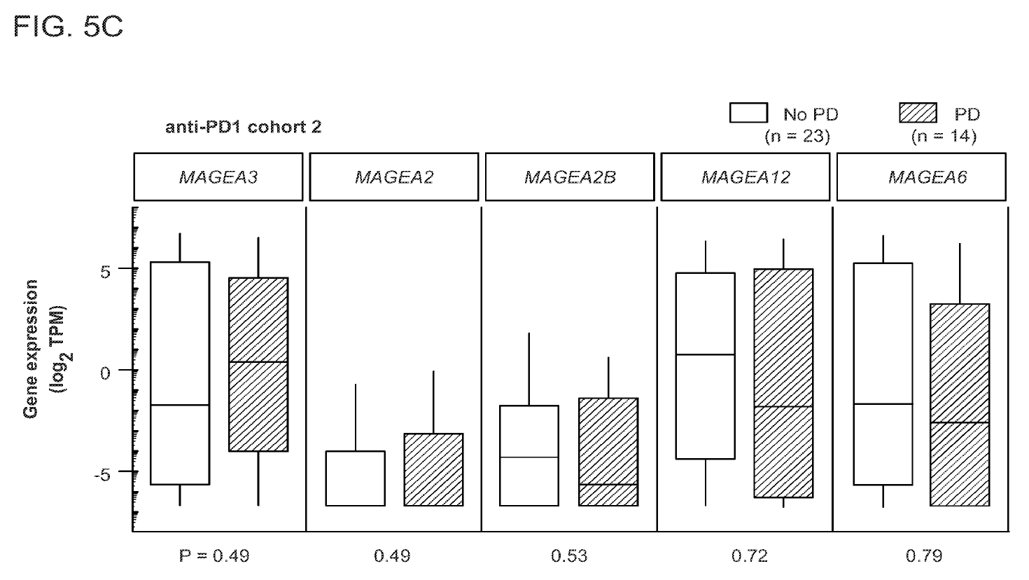

Although studies have suggested common genomic signatures of response (i.e. neoantigen load and clonality) for both CTLA4 and PD1 pathway blockade, the immunobiological processes driven by these two molecules are distinct. Thus, it was hypothesized that the transcriptional signatures of response and resistance to CTLA4 blockade would be unique and not shared with PD1 pathway antagonists. Recently, the genomic and transcriptomic features of response to anti-PD1 therapy in melanomas were reported (Hugo et al., 2016 Cell, 165(1):35-44, incorporated herein by reference). Expression of the Xq28 CTA's and miR-211 were interrogated in these cohorts; however, no correlation with clinical outcome (FIG. 5A-5C) was identified, confirming the hypothesis that the signatures of response and resistance to CTLA4 blockade are unique to anti-CTLA4 therapy and not shared with anti-PD1 therapy. Indeed, molecular signatures of innate resistance to PD1 blockade were also recently shown not to predict resistance to CTLA4 blockade. These results are consistent with the notion that the CTLA4 and PD1 pathways are biologically and clinically distinct.

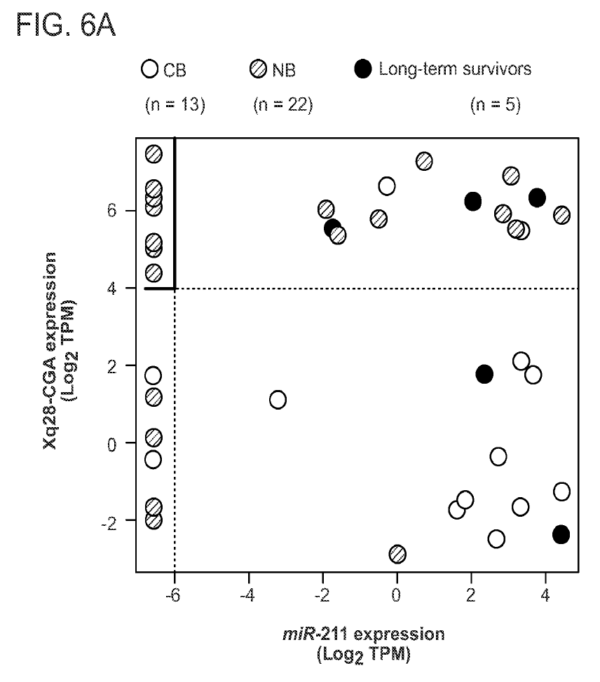

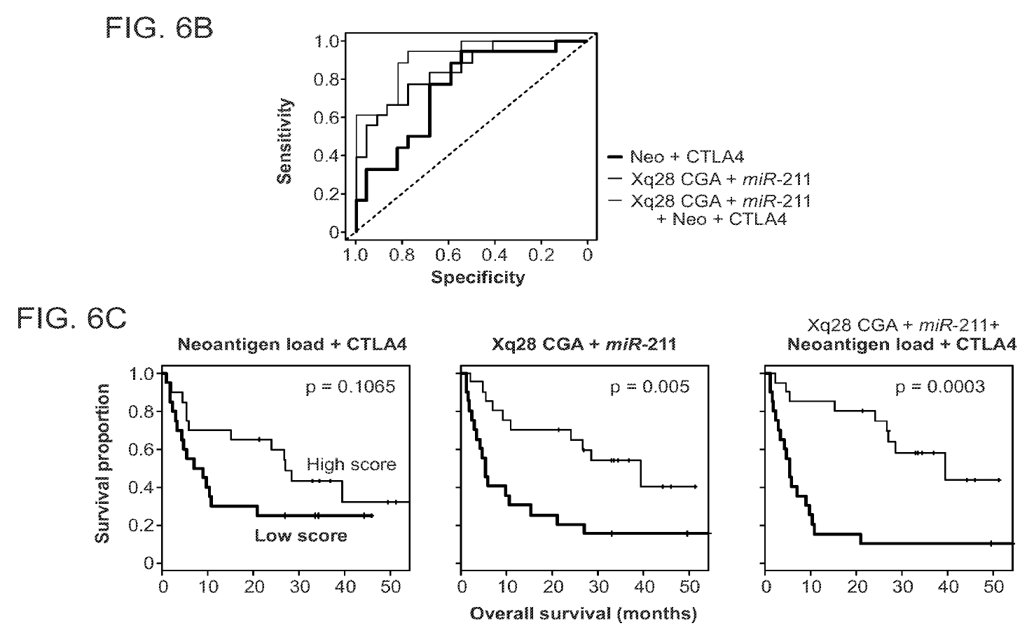

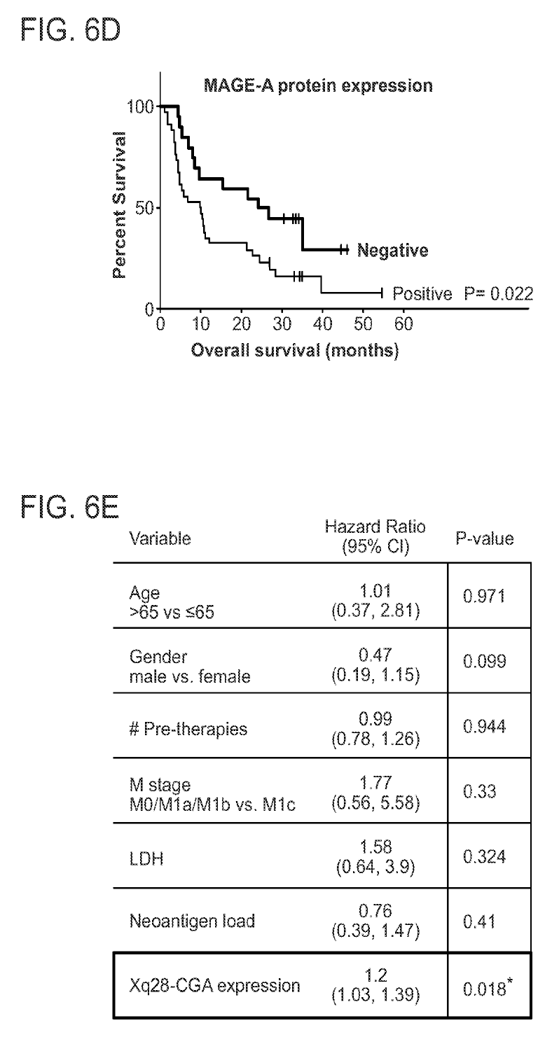

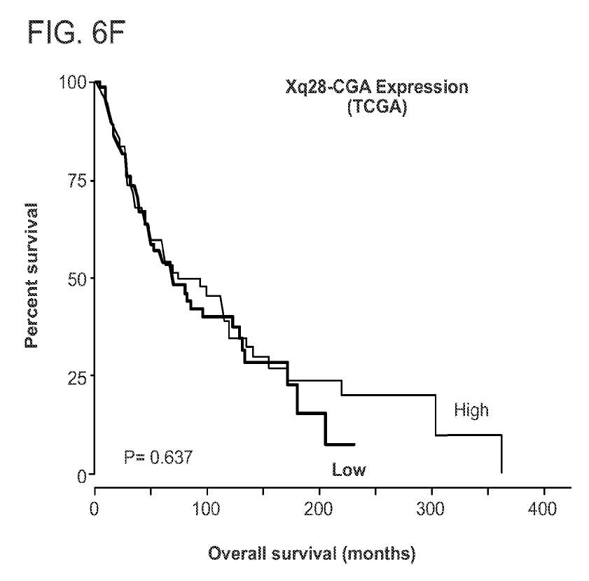

To evaluate the ability of these gene expression signatures to accurately discriminate clinical outcome to CTLA4 blockade, the correlation of the greatest expression value from the Xq28 CGA cluster (comprising genes MAGEA2, MAGEA3, MAGEA6, MAGEA12, CSAG1, GSAG2, and CAG3) was evaluated with expression of miR-211 for all 40 patients from Cohort 1, including the long-term survivors with no clinical benefit, thus examining a “real world” scenario (FIG. 6A). Also, a receiver-operator characteristic (ROC) analysis using the combined classifier of “Xq28+miR-211” was performed. The “Xq28+miR-211” classifier was created by combining expression of the maximally expressed gene in the Xq28 CGA cluster and miR-211 expression in a logistic regression model. Non-responders were characterized by high Xq28 CGA expression and low miR-211 expression. This “Xq28+miR-211” classifier more accurately discriminated patient outcomes than either neoantigen load or CTLA4 expression (FIG. 6B). In fact, AUCs of the latter two reached 0.68 while the new classifier achieved an AUC of 0.85. At 100% sensitivity, using neoantigen load or CTLA4 expression reached only 0% or 27% specificity whereas the new classifier achieved 40% specificity. A Kaplan-Meier analysis of post-ipilimumab overall survival demonstrated no significant effect of combining neoantigen load with CTLA4 gene expression (p=0.1) whereas the Xq28+miR-211 classifier significantly distinguished two clinical groups (p=0.005), especially in addition to both neoantigen load and CTLA4 expression (p=0.0003) (FIG. 6C). In summary, the expression analysis described herein uncovered transcriptomic determinants (i.e., biomarkers) of clinical outcome to CTLA4 blockade that outperform previously identified correlates and illuminate additional mechanisms of response and resistance.

### Example 4: Cancer-Germline Antigens Discriminate Clinical Outcome to CTLA4 Blockade in a Validation Cohort and on the Protein Level

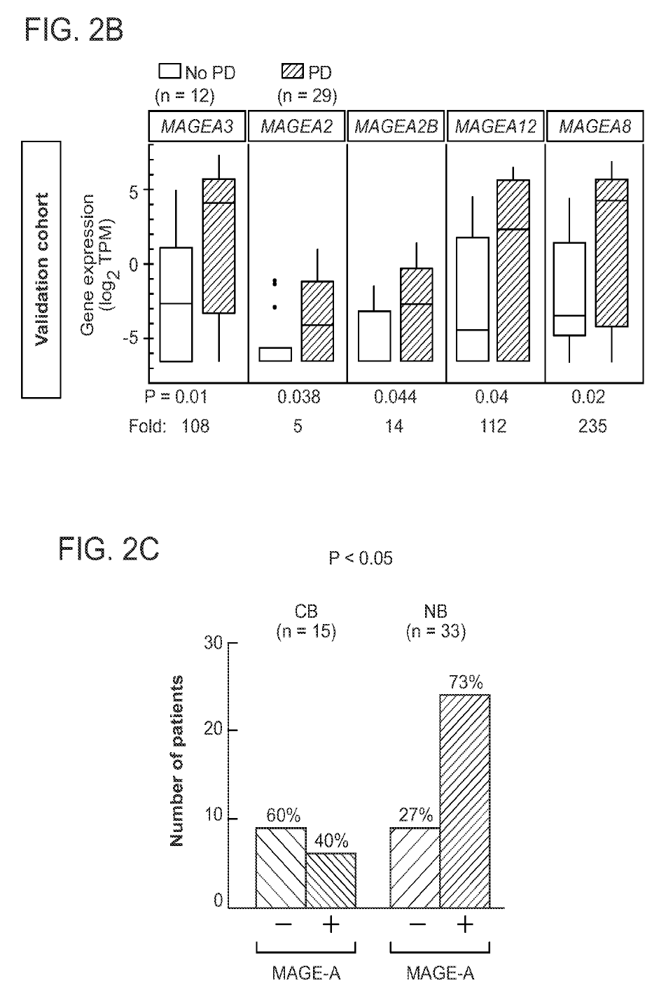

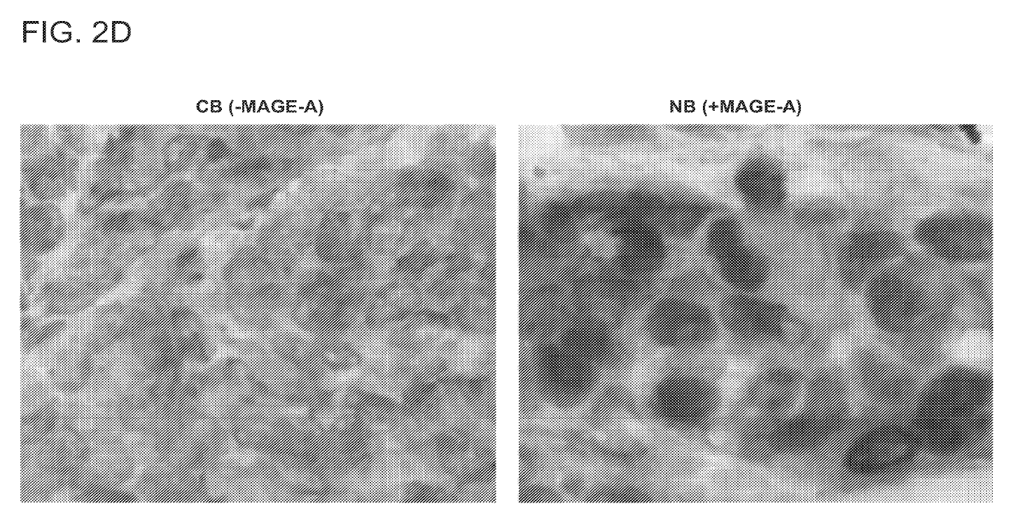

While the discovery cohort was generated from formalin-fixed samples from an observational, retrospective study, the findings were validated in an independent RNA-seq data set generated from cryopreserved tumors from a prospective, randomized trial using pre-treatment patient samples derived from the CheckMate 064 trial (Weber et al., 2016) (see STAR Methods for details). Again, the CRMA genes were amongst the most significantly upregulated genes (FIG. 2A-2B). Because overall survival data attributable to ipilimumab monotherapy was not available given the subsequent administration of nivolumab, the discovery cohort was re-classified based on response assessments used for the CheckMate 064 trial with “progressive disease (PD)” and “no PD” groups. RNA-seq expression values from the validation cohort were available for 5 of 8 genes in the CRMA locus (MAGEA3, MAGEA2, MAGEA2B, MAGEA12, and MAGEA6), and consistent and significant increases were observed in all of these genes in patients with PD in both discovery and validation cohorts (FIG. 2A-2B; 12B-C).

Because of prior reports of discordance between cancer-germline RNA and protein expression across cancers (Chen et al., 2014), immunohistochemistry (IHC) was performed on NB and CB samples using the MAGE-A antibody (clone 6C1), which is broadly reactive for gene products from the MAGE-A family recognizing MAGEA1, A2, A3, A4, A6, A10 and A12. IHC analysis demonstrated that the NB cohort comprised a significantly higher proportion of MAGE-A+ tumors compared to the CB cohort (FIG. 2C-2D, 73% vs 40%, p<0.05), consistent with the RNA-seq analysis. Thus, primary resistance to ipilimumab is strongly associated with baseline RNA and protein expression of a specific cluster of MAGE-A genes.

Detectable MAGE-A protein expression associated with inferior overall survival after ipilimumab therapy (FIG. 6D). In a multivariable analysis of these 40 patients that included evaluation of neoantigen load, CRMA expression emerged as the sole independent risk factor for poor outcome after ipilimumab therapy (Cox proportional-hazards model, p=0.018; FIG. 6E). Finally, CRMA expression did not discriminate overall survival in the untreated TCGA melanoma cohort, suggesting that the CRMA signature is potentially predictive for post-ipilimumab survival rather than prognostic for a clinically aggressive natural history (FIG. 6F). Taken together, these data suggest CRMA expression as a transcriptomic determinant of clinical outcome to CTLA4 blockade.

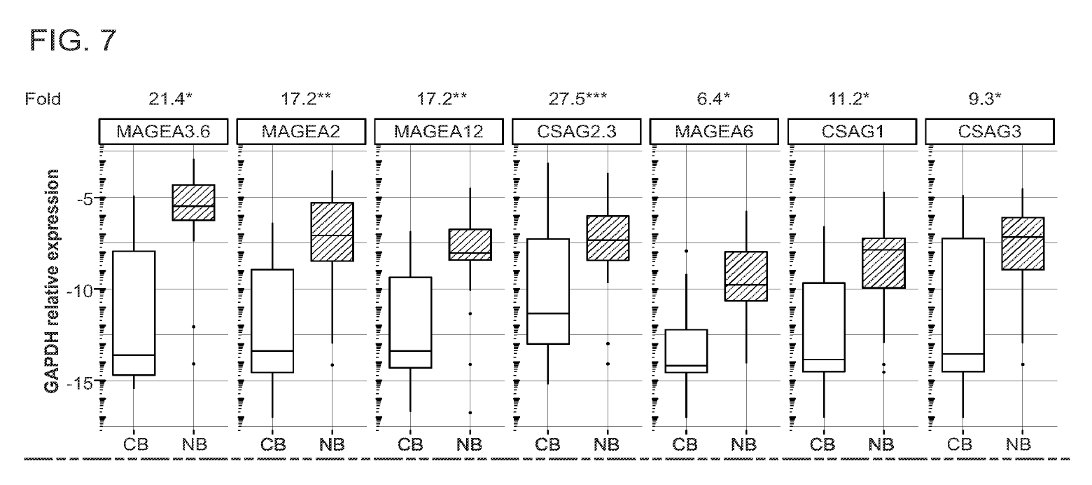

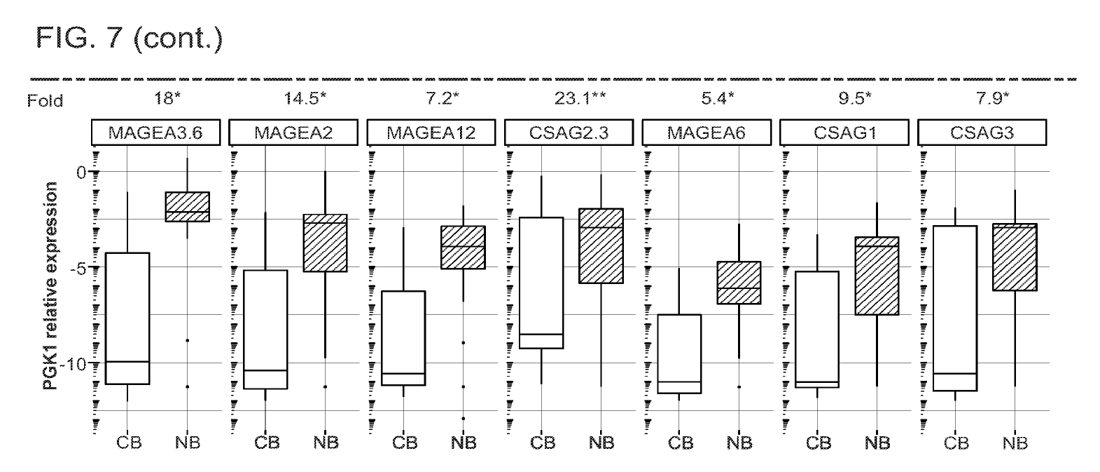

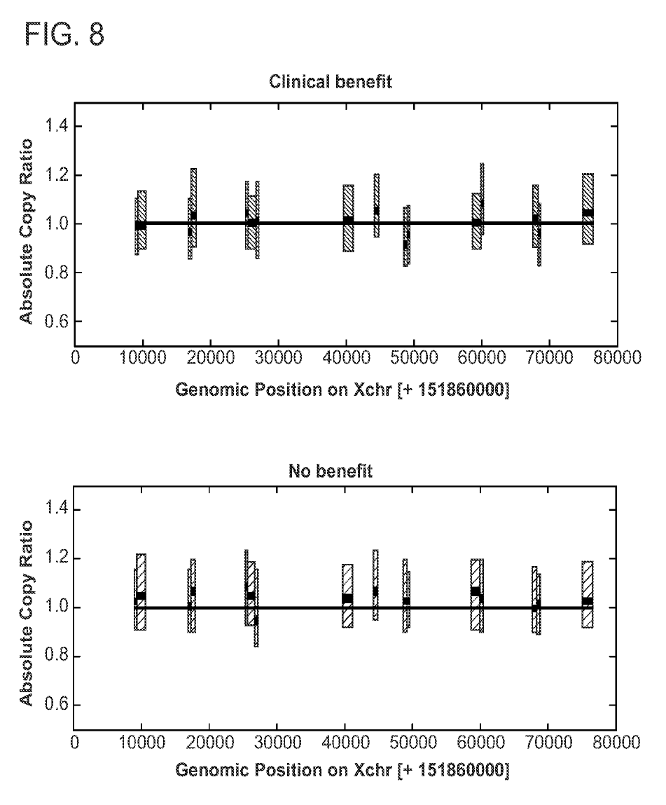

To control for potential artifacts from whole transcriptome RNA-seq (e.g. cDNA library synthesis, read alignment), results were confirmed by gene-specific RT-qPCR of the original tumor RNA in the discovery set using three different housekeeping genes (FIG. 7). To investigate potential mechanisms underlying the transcriptional enrichment of CRMA genes in resistant tumors, copy number variation and DNA methylation at this locus in the clinical trial samples was analyzed. No copy number alterations of this region based on analysis of matched whole-exome sequencing (WES) data were observed (FIG. 8). Neither gender nor prior exposure to cytotoxic therapy (i.e. dacarbazine/temozolomide) was associated with clinical outcome (FIG. 9; 10A). Using the ABSOLUTE algorithm (Carter et al., 2012), similar tumor purity estimates were identified in both patient groups, suggesting that relative enrichment of cancer cells in the NB group was unlikely to be an explanation for this finding (FIG. 10B).

### Example 5: MAGE-A Proteins May Degrade the Danger Molecule HMGB1

Although MAGE proteins have often been studied as immunotherapeutic targets bound to HLA molecules on the cell surface (Van Der Bruggen et al., 2002), recent studies have attributed them with key oncogenic capacities. Expression of MAGE-A3/A6 is necessary for cancer cell viability and can be sufficient to transform cells (Pineda et al., 2015). Critical to the oncogenic functions of MAGEs may be their defining ability to bind to and potentiate the activity of various E3 ubiquitin ligases (Lee and Potts, 2017). MAGE-A2, MAGE-A3, and MAGE-A6 all share specific binding to the TRIM28 ubiquitin ligase. Multiple groups have demonstrated MAGE-TRIM28-induced ubiquitination and proteasomal degradation of the p53 tumor suppressor protein (Doyle et al., 2010) and more recently the AMPK complex which controls cellular metabolic pathways such as autophagy (Pineda et al., 2015).

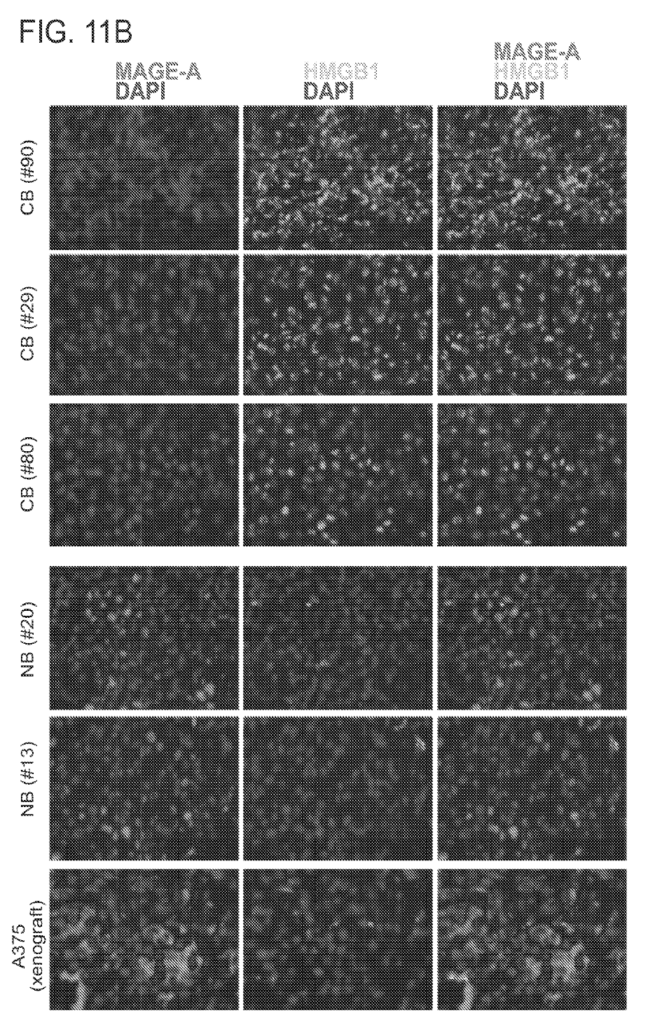

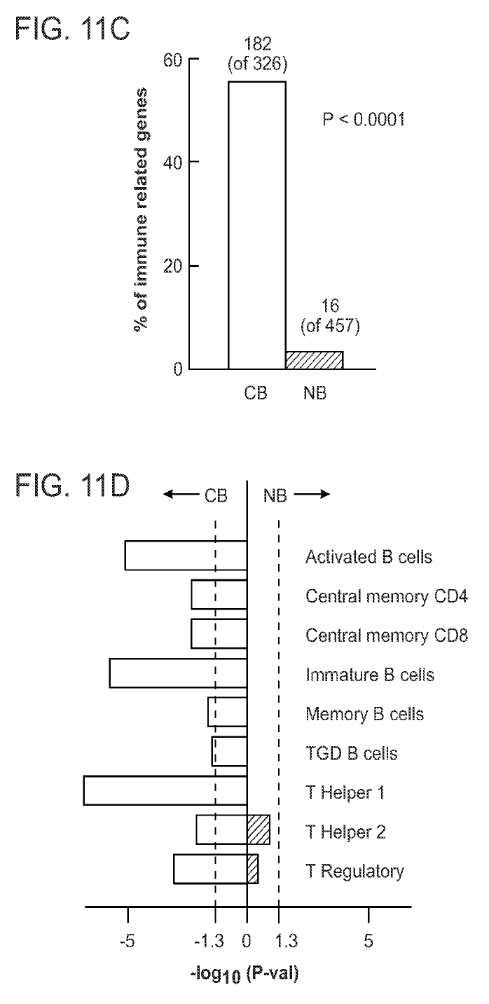

As described herein, the MAGE-A genes within the CRMA locus may target proteins that are involved in immune priming (governed partly by the CTLA4 pathway) rather than immune effector function (governed partly by the PD1 pathway). The results of a screen for direct substrates of MAGE-A through in vitro ubiquitination reactions on protein microarrays containing >9000 recombinant proteins were previously reported (Pineda et al., 2015). As a significantly ubiquitinated target of the MAGE-TRIM28 complex, high-mobility group box 1 (HMGB1) emerged as a likely candidate for its well-described roles in both autophagy and immunogenic cell death that is required for dendritic cell-mediated priming of an adaptive immune response (Apetoh et al., 2007; Tang et al., 2010) (FIG. 11A). To investigate whether HMGB1 was a potential target of the MAGE-TRIM28 complex in melanoma tumors in vivo, immunofluorescence (IF) staining using both anti-HMGB1 and anti-MAGE-A antibodies (along with DAPI) was performed on tumor sections from 5 NB and CB tumors as well as a xenograft from the A375 human melanoma cell line (FIG. 11B). IF staining revealed the mutually exclusive expression of HMGB1 and MAGE-A proteins, with ubiquitous expression of HMGB1 protein in 3 of 3 MAGE-A negative/CB tumors and absent HMGB1 expression in 2 of 2 MAGE-A positive/NB tumors and the A375 human xenograft.

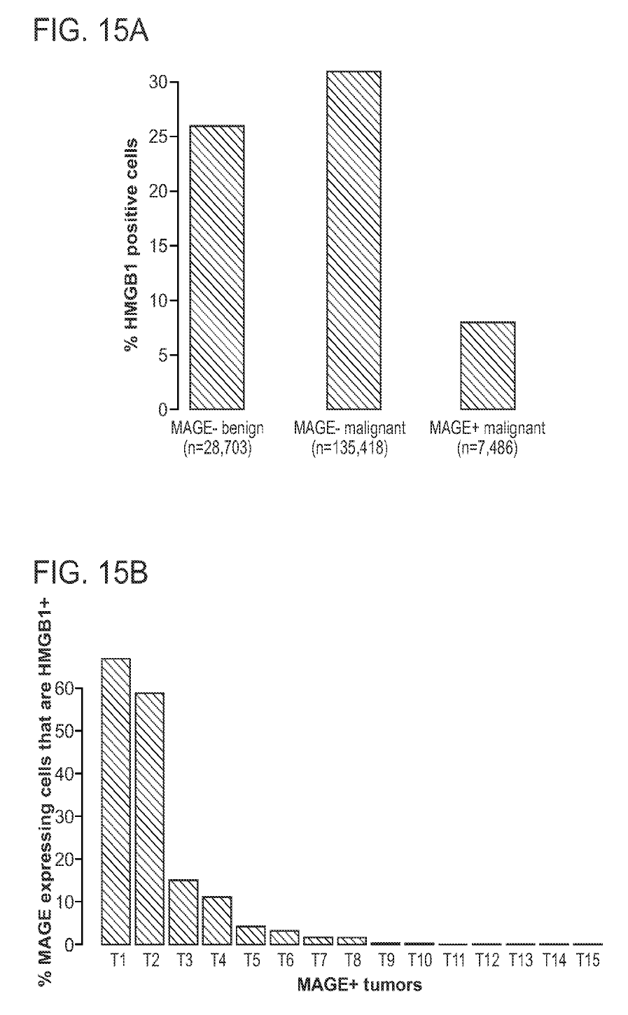

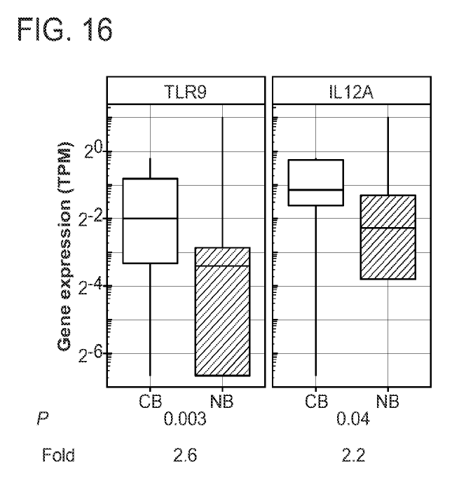

As a well-described damage-associated molecular pattern (DAMP), HMGB1 has been demonstrated to recruit a diverse inflammatory response by binding various toll-like receptors. In particular, HMGB1 can bind dsDNA to form immune complexes recognized by TLR9, resulting in secretion of immunostimulatory cytokines and proliferation of B cells (Avalos et al., 2010; Tian et al., 2007). Consistent with these studies, evidence for an inflammatory, activated immunologic response was identified in the tumor microenvironment of CB samples. Of 326 genes enriched in CB samples, 182 (55%) were identified as immune-related through manual curation, compared to only 16 of 457 (3.5%), in NB samples (Fisher's exact test, p<0.0001; FIG. 11C). In order to test the mutually exclusive expression of MAGE-A and HMGB1 proteins in melanoma, immunofluorescence staining was performed on a melanoma tissue microarray (TMA) comprising 100 samples (9 benign nevi tumors, 91 primary and metastatic melanomas) using antibodies against MAGE and HMGB1. The fraction of HMGB1 positive cells were comparable in MAGE negative cells from the benign nevi and malignant tumors, but was significantly reduced in cells from MAGE malignant samples ((26% and 31% vs 8%, Chi-square test p<2.2×10⁻¹⁶ FIG. 15A). Additionally, in 13 out of 15 melanomas that had any MAGE positive cells, at least 85% of MAGE+ cells lack HMGB1 (FIG. 15B). Supporting the finding that the MAGE-TRIM28 complex degrades HMGB1, significantly decreased expression of HMGB1 pathway genes (TLR9 and IL12A) was identified in no benefit tumors (FIG. 16).

Furthermore, to interrogate immune subpopulations, their involvement was computationally inferred using recently collated gene sets describing specific immune subsets (Angelova et al., 2015). Multiple B and T cell subpopulations were significantly enriched within CB transcriptomes comprising activated, immature and mature B cells along with central memory CD4+ T cells, effector memory CD8+ T cells, T helper 1 and 2 cells, gamma-delta T cells, and T regulatory cells (FIG. 11D). In addition, upregulation of genes related to T cell infiltration, T cell receptor signaling, humoral immunity and macrophage infiltration was also observed (Table 2). No immune subsets were significantly enriched in NB samples. Thus, the CRMA locus may contribute to ipilimumab resistance through targeted destruction of the DAMP, HMGB1, whose absence may restrain the initiation of an adaptive immune response.

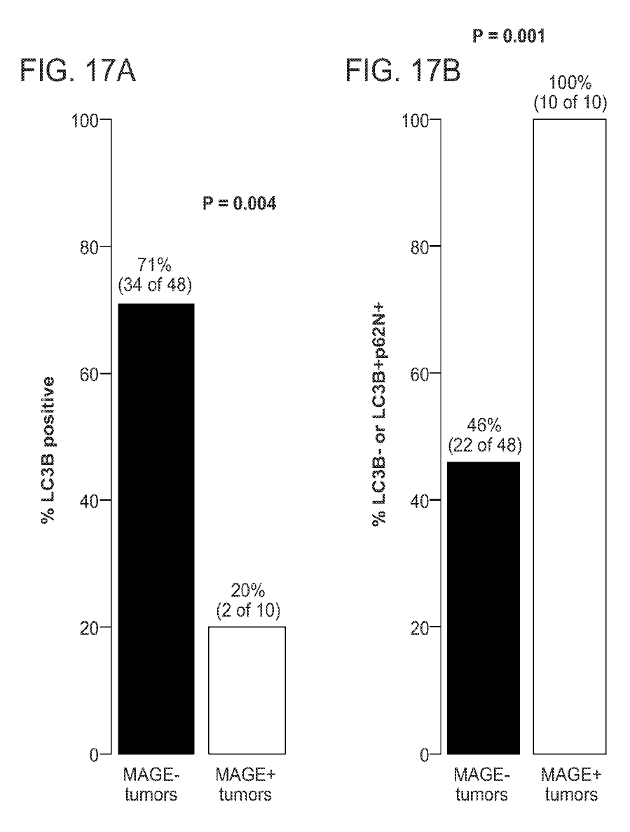

Finally, to ascertain whether the MAGE-TRIM28 complex can suppress autophagy in melanoma, LC3B and p62 staining in MAGE-A stained melanomas was examined. Significantly reduced expression of the autophagy marker LC3B was identified in MAGE-A+ melanomas (FIG. 17A). Moreover, increased evidence of absent or impaired autophagy was found in 100% of MAGE-A positive melanomas (FIG. 17B).

### Discussion

MAGE family members were first identified as targets of anti-tumor T cells in melanoma, and their restricted expression in immune-privileged gonadal tissues and various tumor types highlighted them as immunogenic targets (Coulie et al., 2014; De Plaen et al., 1994; Simpson et al., 2005; van der Bruggen et al., 1991). Therefore, the findings of a specific subcluster of MAGE-A genes overexpressed in melanomas resistant to CTLA4 blockade were unexpected. However, clinical efforts to immunotherapeutically target these proteins have yielded mixed results, suggesting their in vivo immunogenicity should not be assumed (Vansteenkiste et al., 2016). Indeed, many groups have demonstrated the association of CGAs and especially the MAGE family with poor prognostic features in melanoma such as ulceration, thickness, metastases and progression in contrast to the positive prognosis afforded by immune infiltration (Azimi et al., 2012; Barrow et al., 2006; Roeder et al., 2005).

One possible explanation for these results is that reduced Xq28-CGA expression in responding tumors is a manifestation of effective anti-MAGE-A immune activity. Responding melanoma samples are characterized by immune infiltrates that may have already selected against tumor cells expressing high levels of Xq28-CGA genes. However, it was observed that other melanoma antigens previously demonstrated to elicit cellular and humoral responses, such as NY-ESO-1 (another cancer-germline antigen) and various differentiation antigens, showed no evidence of selection in the analysis (FIG. 14). This particular MAGE-A subfamily has not been shown to provoke stronger immune responses than other cancer-germline or melanoma-associated antigens. As described herein, further investigation of in situ immune responses is pursued to rule out this possibility.

An alternative explanation is that these particular Xq28-CGA genes induce immune resistance. Recently described cell-intrinsic functions for MAGE-A3/A6 have implicated these proteins in oncogene addiction, the ubiquitination of key tumor suppressors—notably TP53 and AMPK—that contribute to oncogenesis, and the repression of autophagy (Doyle et al., 2010; Pineda et al., 2015). As described herein, degradation of a protein involved in immune priming (governed in part by the CTLA4 pathway) as opposed to immune effector function (mediated partly by the PD1 pathway) might explain the specificity of the Xq28-CGA cluster to CTLA4, but not PD1, blockade. In fact, an in vitro screen of ubiquitination targets of the MAGE-A3/6-TRIM28 E3 ubiquitin ligase revealed HMGB1, a damage-associated molecular pattern (DAMP) intimately involved in induction of cellular autophagy and immunogenic cell death (FIG. 11A-FIG. 11C) (Apetoh et al., 2007; Scaffidi et al., 2002; Tang et al., 2010; Yanai et al., 2009). Within responding tumor transcriptomes, upregulation of several B and T cell expression signatures were consistent with the immunostimulatory function of HMGB1 (Avalos et al., 2010; Ivanov et al., 2007; Li et al., 2013).

Binding of DAMPs to pattern recognition receptors (such as TLR family members) serve as ‘signal 0’ to kick-start the adaptive immune response through dendritic cell (DC) maturation and migration to the lymph nodes. There, DCs mediate antigen recognition by T cells (‘signal 1’), upregulate costimulatory receptors (‘signal 2’) and secrete polarization and differentiation cytokines (‘signal 3’) (Tang et al., 2012; Yatim et al., 2017). HMGB1 has been identified as a ‘signal 0’ that critically mediates immunogenic cell death—a process that has been proposed to rely on a combination of both antigenicity and adjuvanticity, the former conferred by neo-antigens (in tumors) and the latter provided by specific DAMPs (Galluzzi et al., 2017). Although melanomas have high neoantigen loads that correlate with response to checkpoint blockers, a defect in pathways required for cell death-associated release of DAMPs might decrease the adjuvanticity, and thus the overall immunogenicity, of a tumor. Importantly, both MAGE-A3/A6 and HMGB1 have been demonstrated to induce autophagy (Pineda et al., 2015; Tang et al., 2010), which is necessary for efficient dendritic cell cross-presentation of tumor antigens (Li et al., 2008). Indeed, short-hairpin RNA (shRNA)-mediated knockdown of HMGB1 or essential components of autophagy can abrogate immunogenic cell death (Apetoh et al., 2007; Michaud et al., 2011). Furthermore, loss-of-function polymorphisms in HMGB1-binding receptors or HMGB1 loss from malignant cells associate with poor outcome in patients treated with chemotherapeutic agents known to induce immunogenic cell death (Ladoire et al., 2015) and even in melanoma patients treated with DC-based vaccines (Tittarelli et al., 2012). Consequently, disabling the emission of danger signals such as HMGB1 may allow Xq28-CGA-expressing melanomas to inhibit the initiation of an adaptive immune response and impede the efficacy of CTLA4 blockade. Careful dissection of the role of MAGE-A-HMGB1 interactions in mediating outcome to CTLA4 blockade unveils new strategies to improve clinical responses to ipilimumab, for example through combination with HMGB1 receptor agonists.

Although the statistical stringency was relaxed because of the small discovery cohort, the finding of Xq28-CGA gene upregulation in primary resistance to CTLA4 blockade was validated through confirmation in a prospective, independent cohort and technical verification by qPCR and immunohistochemistry. Because both CTLA4 blockade and cancer vaccines impact immune priming and memory formation, the results presented herein may also explain the long history of unsuccessful cancer vaccination efforts targeting MAGEA3 and MAGEA6 (Palucka and Banchereau, 2014; Pedicord et al., 2011; Saiag et al., 2016; Vansteenkiste et al., 2016). The results presented herein also indicate that mechanisms of response and resistance to immune priming (e.g. CTLA4 blockade) may differ substantially from those relevant to clinical manipulation of effector immunity (e.g. PD1/PD-L1 blockade). As immunotherapeutic combinations are increasingly evaluated, understanding these mechanisms is important for precisely pairing patients with appropriate combinations to avoid toxicity and ensure efficacy. Nevertheless, these findings are investigated in larger, prospective cohorts to evaluate these signatures as potential biomarkers of outcome, and studied in preclinical models as potential therapeutic targets to sensitize to or combine with CTLA4 blockade.

### STAR Methods

The following materials and methods were used in this example.

### Study Design

A previously reported RNA-seq dataset of pre-therapy samples collected from a study cohort of 40 melanoma patients treated with ipilimumab (Van Allen et al., 2015) was analyzed. In this study, RNA and genomic DNA were extracted from formalin-fixed, paraffin-embedded (FFPE) tumor blocks, and Illumina's TruSeq Stranded Total RNA Sample Prep Kit was used to generate RNA-seq libraries. Patient classification was maintained from the original report (Table 1). The “clinical benefit” (CB) group (n=13) was defined as patients who achieved complete or partial response by RECIST criteria, or stable disease by RECIST criteria with overall survival greater than one year. The “no benefit” (NB) group (n=22) was defined as patients who had progressive disease by RECIST criteria or stable disease with overall survival less than 1 year. A third group of five patients was described with early progression on ipilimumab (progression-free survival <6 months) but overall survival exceeding 2 years. To identify genes associated with clinical benefit and no benefit, the differential expression analysis between the CB and NB groups was performed. The association of Xq28-CGA expression with survival outcome was evaluated in the entire cohort. Genes were identified as differentially expressed when their median expression differed by more than two-fold with a nominal one-sided p-value≤0.05 (Wilcoxon test).

An independent, validation cohort comprised 41 patients from the CheckMate 064 trial (Weber et al., 2016) treated with ipilimumab followed by nivolumab (Table 1). The trial also studied in parallel a cohort comprising patients treated with the reverse sequence of nivolumab followed by ipilimumab. Overall survival could not be assessed in this crossover design. Response assessments, collected at week 13 before the planned switch, were used to classify patients into either no progressive disease (“No PD”; comprising stable disease, complete response and partial response, n=12) or progressive disease (“PD”; n=29) from each arm. Tumor samples were cryopreserved in RNALater. RNA-seq libraries were generated using the Stranded TruSeq method, and 75 bp paired-end reads for duplexed samples were sequenced per lane (Expression Analysis, Inc; Morrisville, N.C.). RNA-Seq and associated clinical data were available for the following Xq28-CGA genes: MAGEA3, MAGEA2, MAGEA2B, MAGEA12, MAGEA6.

Xq28-CGA expression was evaluated in patients from two different anti-PD1-treated cohorts. Anti-PD1 cohort 1 comprised 28 pre-anti-PD1-treated tumors (Hugo et al., 2016); anti-PD1 cohort 2 comprised 37 pre-treatment tumors from the nivolumab followed by ipilimumab arm of the CheckMate064 trial (Hugo et al., 2016; Weber et al., 2016) (Table 1).

465 melanoma samples from TCGA (Cancer Genome Atlas, 2015) were used to further investigate identified gene expression and methylation signatures.

### Processing and Analysis of Sequencing Data

RNA sequencing data from the discovery cohort was aligned to the reference human genome with STAR (Dobin et al., 2013), followed by removal of duplicates and quantification with RSEM (Li and Dewey, 2011). RNA sequencing data from the CheckMate064 trial was first aligned using STAR (Dobin et al., 2013) followed by removal of duplicate reads. Gene level quantification of the reads was performed with the htseq-count tool (Anders et al., 2015).

Whole exome data for 110 patients from the discovery cohort (including the 40 with transcriptomic data) (Van Allen et al., 2015) and Infinium 450K methylation chip data for 476 samples from TCGA (Cancer Genome Atlas, 2015) was also obtained.

Identification of Genes Associated with Xq28-CGA Expression in TCGA

A metagene was defined as one comprising the following Xq28-CGA genes: MAGEA2, MAGEA3, MAGEA6, MAGEA12, CSAG1, CSAG2, and CSAG3 (MAGEA2B was not quantified by TCGA). The expression of this metagene was defined as the geometric mean of its components and was computed for each of 465 TCGA melanoma samples. TCGA samples with expression values in the bottom and top quartiles for this metagene were classified into “Xq28-CGA-low” (n=117) and “Xq28-CGA-high” (n=116) groups respectively. An unbiased gene expression analysis between these two groups was performed using one-sided Wilcoxon tests with a false discovery rate (FDR) threshold of 0.05 and two-fold change threshold.

### Validation of Genes by Quantitative Polymerase Chain Reaction

Expression of target genes in the discovery cohort (Van Allen et al., 2015) from RNA that was extracted for RNA-sequencing was validated. TaqMan gene expression assays (Applied Biosystems, Foster City, Calif.) was used and cDNA amplification was performed using the TaqMan Gene Expression Master Mix (Applied Biosystems) on an Applied Biosystems 7500HT Fast real-time polymerase chain reaction (PCR) System (10-minute enzyme activation and 40 cycles of 15 s at 95° C., 1 minute at 60° C.). Samples were measured in duplicate; “undetermined” values were assigned a cycle threshold (Ct) of 40. HPRT1, GAPDH, and PGK1 were used as housekeeping genes to calculate relative expression values according to the delta-Ct method.

### Identification of Differentially Methylated Probes Between Xq28-CGA-Low and -High Groups in TCGA

Level3 Infinium 450K methylation chip data was retrieved from TCGA for “Xq28-CGA-low” (n=117) and “Xq28-CGA-high” (n=116) groups. A probe-level comparison was performed between the two groups using Wilcoxon tests with an FDR of 0.05 for all 485,577 CpG probes. A probe with higher median beta values in one group was considered relatively hypermethylated in that group compared to the other.

### Copy Number Analysis

The clinical benefit (CB) and no benefit (NB) groups were tested for variations in germline and somatic CNVs in Xq28 locus using GATK 4 target coverage denoising and ACNV pipelines. Raw coverage on whole exome Agilent targets for 110 normal and tumor samples was collected and GC bias was corrected for. The 40 samples with RNA-seq data used in this study were set aside and the remaining 70 samples were used to learn the target coverage bias profile (“panel of normals”). Then, the coverage profile of samples used in this study were denoised and normalized using the obtained panel of normals. Tumor samples with anomalously low signal-to-noise ratio and normal samples with significant contamination were detected and excluded from the analysis. Empirical distributions of raw copy ratios on all 16 Agilent targets in the Xq28 locus were calculated using an agnostic prior distribution and copy ratio likelihoods for each sample. Absolute copy ratios with respect to diploid were estimated by performing allelic CNV analysis, detecting copy neutral autosomal intervals, and normalizing the raw copy ratios accordingly.

The two groups were tested for germline and somatic copy number variations in the Xq28 locus using the two-sample KS test. The test was performed separately for each target, and for the copy ratio average on all 16 targets in the Xq28 locus. The copy ratio distributions in each case were identified via empirical bootstrap.

### Amplicon Methylation Analysis

Genomic DNA samples of male patients was bisulfite-treated (EZ DNA Methylation-Gold™ Kit, Zymo Research) and individual amplicons were amplified via PCR (using TaKaRa EpiTaq™ HS, Clonetech). The following primer pairs were used: MAGEA3, MAGEA6 and MAGEA12 gene body, Forward Primer: GATTGTGTTTTTGAGGAGAAAATTT (SEQ ID NO: 206), Reverse Primer: CTCCCACTAACCCTAACTACAACTC (SEQ ID NO: 207). MAGEA3 and MAGEA6 gene promoter, Forward Primer: AATTTTAGGATTTTGAGGGATGAT (SEQ ID NO: 208), Reverse Primer: AAACCCTCTATCTAAAATAAAACCC (SEQ ID NO: 209). PCR products were subcloned (One Shot® TOP10 Chemically Competent E. coli, NEB) and individual colonies were sequenced for subsequent methylation analysis. For the local regression analysis (R package ‘msir’) the span smoothing parameter for loess was set to 0.4.

### Immunohistochemistry (IHC) and Immunofluorescence (IF)

All specimens were evaluated by conventional histopathology. Antibodies used for IHC and IF included mouse anti-MAGE antibody (6C1; Santa Cruz Biotechnology, San Diego, Calif. USA) and rabbit anti-HMGB1 antibody (ab18256; Abcam, Cambridge, Mass.). Immunohistochemistry was performed with pressure cooker heat-induced epitope retrieval on 4-mm-thick sections prepared from formalin-fixed, paraffin-embedded tissues. In addition to detection of biomarker antibodies by use of chromogen vector NovaRed peroxidase substrate (Vector laboratory, Burlingame, Calif., USA), selected samples were evaluated by a dual labeling approach by combining NovaRed with a blue chromogen vector Blue AP substrate (Vector laboratory). Positive and negative tissue controls and isotype-specific irrelevant antibody controls were used to ensure specificity. Consistent with other reports of IHC for MAGE-A protein, nuclear and/or cytoplasmic staining was interpreted as a positive staining pattern; staining in any cancer cells, irrespective of percentage of positive cells or intensity, was regarded as positive.

Dual-labeling immunoflourescence was performed to complement immunohistochemistry as a means of two-channel identification of epitopes co-expressed in similar or overlapping sub-cellular locations. Briefly, 4-mm-thick paraffin sections were incubated with 1:100 mouse anti-MAGE antibody+1:1000 rabbit anti-HMGB1 antibody at 4° C. overnight and then incubated with 1:2000 Alexa Fluor 594-conjugated anti-mouse IgG and Alexa Fluor 488-conjugated anti-rabbit IgG (Invitrogen) at room temperature for 1 hour. The sections were cover slipped with ProLong Gold anti-fade with DAPI (Invitrogen). Sections were analyzed with a BX51/BX52 microscope (Olympus America, Melville, N.Y., USA), and images were captured using the CytoVision 3.6 software (Applied Imaging, San Jose, Calif., USA). Single label immunofluorescence was also performed using isotype-specific irrelevant primary antibodies and with switching of the secondary antibodies to ensure specificity and exclude cross reactivity.

### Survival Analysis

The association of Xq28-CGA expression with overall survival was evaluated using the Kaplan-Meier method. In the discovery cohort, patients with Xq28-CGA expression values above the median were considered “high” and below the median were considered “low.” The effect of Xq28-CGA expression on overall survival adjusting for age, gender, number of pre-therapies, M-stage, LDH and neoantigen load was assessed using the Cox proportional hazards model.

### Statistical Analysis

Differential expression and methylation analyses within TCGA samples were performed using a false discovery rate (Benjamini-Hochberg) of 0.05. Hypergeometric tests were used to evaluate overlap of differentially expressed genes between the clinical and TCGA cohorts. Multivariable survival analysis was performed using the Cox proportional hazards model (R package ‘coxph’). All statistical analyses were done using R version-3.2.5. Overlaps of gene lists with pathways in the PANTHER (Protein ANalysis THrough Evolutionary Relationships) database (containing 177 pathways) were evaluated with the overrepresentation test using the Bonferroni correction (Mi et al., 2016).

TABLE 1
Clinical characteristics of discovery and validation cohorts

| | | | | | Time to | Overall | | | |
|---|---|---|---|---|---|---|---|---|---|
| | | | | | progression | survival | | | # prior |
| | ID | Gender | Age | RECIST | (wks) | (yrs) | LDH | M class | therapies |
| Discovery Cohort | Pat02 | female | 42 | SD | 77 | 4.5 | 1 | M1c | 1 |
| (Van Allen et al): | Pat04 | male | 71 | PR | 92 | 2.7 | 0 | M1b | 7 |
| Clinical benefit (CB) | Pat29 | male | 82 | X | 49 | 3.6 | 0 | M1c | 1 |
| | Pat38 | male | 45 | PR | 43 | 4.2 | 0 | M1c | 2 |
| | Pat39 | male | 67 | CR | 212 | 4.1 | 0 | M1b | 1 |
| | Pat47 | male | 78 | CR | 158 | 3 | 0 | M1c | 1 |
| | Pat49 | male | 36 | SD | 23 | 2.8 | 0 | M1c | 0 |
| | Pat79 | male | 69 | PR | 58 | 2.2 | 0 | M1b | 1 |
| | Pat80 | male | 48 | SD | 26 | 2 | 1 | M1c | 1 |
| | Pat88 | female | 60 | SD | 95 | 2.7 | NA | M1c | 0 |
| | Pat90 | male | 59 | PR | 96 | 2.8 | 0 | M1c | 1 |
| | Pat123 | female | 50 | SD | 78 | 2.3 | 1 | M1c | 5 |
| | Pat126 | male | 77 | PR | 27 | 1.8 | 1 | M1b | 1 |
| Discovery Cohort | Pat03 | female | 61 | PD | 11 | 0.3 | 1 | M1c | 3 |
| (Van Allen et al): | Pat06 | male | 33 | PD | 11 | 0.4 | 0 | M1c | 4 |
| No benefit (NB) | Pat08 | male | 73 | PD | 10 | 0.4 | 0 | M1c | 2 |
| | Pat118 | female | 48 | PD | 12 | 0.4 | 0 | M1c | 3 |
| | Pat14 | male | 32 | PD | 4 | 0.1 | 1 | M1c | 4 |
| | Pat15 | male | 59 | PD | 3 | 0.5 | 0 | M1c | 0 |
| | Pat19 | male | 78 | PD | 10 | 0.3 | 0 | M1c | 1 |
| | Pat25 | male | 69 | PD | 10 | 0.9 | 1 | M1c | 1 |
| | Pat33 | male | 65 | PD | 30 | 0.6 | 1 | M1c | 3 |
| | Pat36 | female | 52 | PD | 6 | 0.1 | 1 | M1c | 3 |
| | Pat37 | female | 47 | PD | 3 | 0.2 | 1 | M1c | 1 |
| | Pat40 | male | 74 | PD | 5 | 0.1 | 1 | M1c | 3 |
| | Pat41 | male | 64 | PD | 8 | 0.4 | 1 | M1c | 1 |
| | Pat43 | female | 75 | PD | 5 | 0.1 | 1 | M1b | 2 |
| | Pat44 | female | 57 | PD | 9 | 0.7 | 1 | M1c | 1 |
| | Pat45 | male | 68 | PD | 9 | 0.2 | 1 | M1c | 1 |
| | Pat46 | female | 36 | PD | 5 | 0.4 | 0 | M1b | 1 |
| | Pat50 | male | 77 | PD | 9 | 0.2 | 0 | M1c | 0 |
| | Pat81 | female | 54 | PD | 11 | 1.7 | 0 | M1a | 0 |
| | Pat85 | male | 83 | PD | 12 | 1.3 | 0 | M1c | 1 |
| | Pat86 | male | 55 | SD | 22 | 0.8 | 0 | M1a | 0 |
| | Pat98 | female | 57 | PD | 11 | 0.4 | 0 | M1c | 0 |
| Discovery Cohort | Pat119 | female | 68 | PD | 12 | 2.2 | 0 | M1b | 1 |
| (Van Allen et al): | pat16 | male | 61 | PD | 20 | 3.8 | 0 | M1c | 1 |
| Long-term survival | Pat27 | male | 77 | PD | 11 | 3.2 | 1 | M1c | 2 |
| with no clinical | Pat28 | male | 22 | PD | 15 | 2.8 | 0 | M1c | 0 |
| benefit | Pat83 | male | 61 | PD | 15 | 2.2 | 1 | M0 | 1 |

| | ID | Gender | Age | RECIST |
|---|---|---|---|---|
| Validation cohort | 1 | Male | 47 | SD |
| (Weber et al)Ipilimumab | 2 | Male | 82 | PR |
| --> nivolumab: No | 3 | Male | 31 | PR |
| progressive disease | 4 | Male | 70 | SD |
| | 5 | Female | 48 | SD |
| | 6 | Male | 66 | SD |
| | 7 | Male | 66 | PR |
| | 8 | Male | 51 | SD |
| | 9 | Male | 74 | SD |
| | 10 | Male | 67 | PR |
| | 11 | Male | 54 | SD |
| | 12 | Male | 63 | SD |
| Validation cohort | 13 | Male | 84 | PD |
| (Weber et al)Ipilimumab | 14 | Male | 37 | PD |
| --> nivolumab: | 15 | Male | 46 | PD |
| Progressive disease | 16 | Female | 64 | PD |
| | 17 | Male | 73 | PD |
| | 18 | Male | 74 | PD |
| | 19 | Male | 56 | PD |
| | 20 | Male | 78 | PD |
| | 21 | Female | 62 | PD |
| | 22 | Female | 55 | PD |
| | 23 | Female | 75 | PD |
| | 24 | Female | 44 | PD |
| | 25 | Female | 63 | PD |
| | 26 | Male | 38 | PD |
| | 27 | Male | 53 | PD |
| | 28 | Male | 36 | PD |
| | 29 | Female | 35 | PD |
| | 30 | Male | 55 | PD |
| | 31 | Male | 74 | PD |
| | 32 | Female | 78 | PD |
| | 33 | Male | 79 | PD |
| | 34 | Male | 84 | PD |
| | 35 | Male | 66 | PD |
| | 36 | Female | 56 | PD |
| | 37 | Male | 64 | PD |
| | 38 | Female | 63 | PD |
| | 39 | Female | 73 | PD |
| | 40 | Male | 52 | PD |
| | 41 | Male | 75 | PD |
| Validation cohort | 34 | Male | 65 | PR |
| (Weber et al) Nivolumab | 35 | Female | 27 | PR |
| --> ipilimumab: No | 36 | Female | 42 | PR |
| progressive disease | 37 | Male | 74 | PR |
| | 38 | Male | 58 | SD |
| | 39 | Male | 57 | SD |
| | 40 | Female | 64 | PR |
| | 41 | Female | 40 | SD |
| | 42 | Male | 62 | PR |
| | 43 | Male | 63 | PR |
| | 44 | Male | 36 | SD |
| | 45 | Male | 46 | SD |
| | 46 | Male | 73 | SD |
| | 47 | Male | 57 | SD |
| | 48 | Male | 60 | PR |
| | 49 | Male | 52 | SD |
| | 50 | Female | 58 | SD |
| | 51 | Female | 47 | PR |
| | 52 | Female | 59 | PR |
| | 53 | Male | 89 | SD |
| | 54 | Male | 72 | PR |
| | 55 | Male | 56 | PR |
| | 56 | Male | 81 | PR |
| Validation cohort | 57 | Male | 62 | PD |
| (Weber et al)Nivolumab | 58 | Female | 62 | PD |
| --> ipilimumab: | 59 | Female | 72 | PD |
| Progressive disease | 60 | Male | 73 | PD |
| | 61 | Female | 49 | PD |
| | 62 | Male | 40 | PD |
| | 63 | Male | 71 | PD |
| | 64 | Female | 32 | PD |
| | 65 | Male | 36 | PD |
| | 66 | Male | 60 | PD |
| | 67 | Male | 70 | PD |
| | 68 | Male | 30 | PD |
| | 69 | Male | 52 | PD |
| | 70 | Male | 77 | PD |

TABLE 2
Genes enriched in No Benefit and Clinical Benefit group

| Gene | Gene Nome | Fold Change | P-value | Group |
|---|---|---|---|---|
| MAGEA2 | melanoma antigen family A, 2 | 194.87 | 0.001681771 | NB |
| AC093787.1 | | 191.37 | 0.014074651 | NB |
| KRT8P8 | keratin 8 pseudogene 8 | 115.36 | 0.000437919 | NB |
| CSAG4 | CSAG family, member 4 (pseudogene) | 113.21 | 0.001374253 | NB |
| GABRA3 | gamma-aminobutyric add (GABA) A receptor, alpha 3 | 110.18 | 0.00105356 | NB |
| MAGEA2B | melanoma antigen family A, 2B | 102.86 | 0.001902564 | NB |
| CSAG2 | CSAG family, member 2 | 95.83 | 0.000939032 | NB |
| MKRN9P | makorin ring finger protein 9, pseudogene | 93.52 | 0.001122703 | NB |
| MAGEA6 | melanoma antigen family A, 6 | 87.63 | 0.005477973 | NB |
| CSAG3 | CSAG family, member 3 | 87.39 | 0.001254778 | NB |
| RP1-273G13.1 | | 84.57 | 0.001749305 | NB |
| EYA1 | eyes absent homolog 1 (Drosophila) | 79.9 | 0.003059735 | NB |
| MIR218-1 | microRNA 218-1 | 77.65 | 0.004961686 | NB |
| CSAG1 | chondrosarcoma associated gene 1 | 73.33 | 0.008425839 | NB |
| RP11-379D21.3 | | 67.64 | 0.003911124 | NB |
| MAGEA12 | melanoma antigen family A, 12 | 67.35 | 0.013100955 | NB |
| MAGEA3 | melanoma antigen family A, 3 | 58.31 | 0.008391308 | NB |
| RP11-360D2.1 | | 55.78 | 0.004483201 | NB |
| MIR1262 | micraRNA 1262 | 48.86 | 0.004516422 | NB |
| PSG6 | pregnancy specific beta-1-glycoprotein 6 | 47.93 | 0.009417526 | NB |
| PSG11 | pregnancy specific beta-1-glycoprotein 11 | 42.47 | 0.001216848 | NB |
| RP1-13D10.2 | | 40.78 | 0.002072617 | NB |
| OR11H12 | olfactory receptor, family 11, subfamily H, member 12 | 39.49 | 0.004790916 | NB |
| MAGEC1 | melanoma antigen family C, 1 | 37.21 | 0.024814077 | NB |
| CTD-2302A16.2 | | 36.28 | 0.038385003 | NB |
| CTD-2201G16.1 | | 36.19 | 0.025889607 | NB |
| RP11-526L8.1 | | 34.16 | 0.026158447 | NB |
| RP11-804F13.2 | | 34.02 | 0.001073914 | NB |
| PSG8 | pregnancy specific beta-1-glycoprotein 8 | 31.38 | 0.02875596 | NB |
| SOX5P | SRY (sex determining region Y)-box 5 pseudogene | 27.52 | 0.010553127 | NB |
| AC097635.5 | | 27.14 | 0.013029879 | NB |
| SLC25A15P4 | solute carrier family 25 (mitochondrial carrier; ornithin [symbol] | 26.67 | 0.033214335 | NB |
| RP11-533K9.3 | | 25.7 | 0.025889607 | NB |
| NFYAP1 | nuclear transcription factor Y, alpha pseudogene 1 | 25.61 | 0.007926133 | NB |
| GJB6 | gap junction protein, beta 6, 30 kDa | 25.6 | 0.002430921 | NB |
| GABRQ | gamma-aminobutyric acid (GABA) A receptor, theta | 25.08 | 0.000665219 | NB |
| XIST | X inactive specific transcript (non-protein coding) | 24.53 | 0.015742933 | NB |
| TNP1 | transition protein 1 (during histone to protamine repla [symbol] | 23.74 | 0.030404894 | NB |
| AC135995.2 | | 23.56 | 0.015640563 | NB |
| SNORA27 | small nucleolar RNA, H/ACA box 27 | 22.87 | 0.014772695 | NB |
| SERPINA5 | serpin peptidase inhibitor, clade A (alpha-1 antiprotein [symbol] | 22.55 | 0.001957165 | NB |
| RP11-250B2.2 | | 21.76 | 0.003178944 | NB |
| CTC-329D1.2 | | 21.66 | 0.010553127 | NB |
| MAGEA1 | melanoma antigen family A, 1 (directs expression of an [symbol] | 21.42 | 0.016995145 | NB |
| RP11-379D21.2 | | 20.89 | 0.001703467 | NB |
| OR2M3 | olfactory receptor, family 2, subfamily M, member 3 | 20.48 | 0.00982579 | NB |
| AGMO | alkylglycerol monooxygenase | 20.21 | 0.001148356 | NB |
| RP11-728C8.1 | | 20.06 | 0.008215814 | NB |
| CYP26A1 | cytochrome P450, family 26, subfamily A, polypeptide [symbol] | 19.61 | 0.008358255 | NB |
| RP11-685G9.2 | | 19.57 | 0.003476545 | NB |
| RP1-221C16.7 | | 19.51 | 0.012379028 | NB |
| ENSAP1 | endosulfine alpha pseudogene 1 | 19.32 | 0.042437855 | NB |
| PSG5 | pregnancy specific beta-1-glycoprotein 5 | 19.2 | 0.009622364 | NB |
| AC007312.3 | | 19.11 | 0.038857039 | NB |
| LA16c-4G1.5 | | 19.11 | 0.004269868 | NB |
| RP11-114H24.7 | | 19.09 | 0.039174691 | NB |
| PSG2 | pregnancy specific beta-1-glycoprotein 2 | 19.08 | 0.014252125 | NB |
| AC126339.2 | | 18.9 | 0.044112433 | NB |
| RP4-535B20.1 | | 18.85 | 0.049098599 | NB |
| RP11-290F24.3 | | 18.84 | 0.002622662 | NB |
| PPM1AP1 | protein phosphatase, Mg2+/Mn2+dependent 1A, pseu [symbol] | 18.79 | 0.020779381 | NB |
| HMGN1P17 | high mobility group nucleosome binding domain 1 pse [symbol] | 18.67 | 0.023187328 | NB |
| PSG10P | pregnancy specific beta-1-glycoprotein 10, pseudogene | 18.44 | 0.011708098 | NB |
| OR7E156P | olfactory receptor, family 7, subfamily E, member 156 [symbol] | 18.38 | 0.023557449 | NB |
| RP4-610C12.3 | | 18.33 | 0.004753871 | NB |
| FPGT-TNNI3K | FPGT-TNNI3K readthrough | 18.05 | 0.008875911 | NB |
| RP4-710M3.1 | | 17.9 | 0.030948543 | NB |
| AC010724.2 | | 17.64 | 0.038448428 | NB |
| RP11-17A1.2 | | 17.54 | 0.002310348 | NB |
| VENTXP5 | VENT homeobox pseudogene 5 | 17.49 | 0.000887029 | NB |
| CFHR4 | complement factor H-related 4 | 17.16 | 0.00659527 | NB |
| CCDC42 | coiled-coil domain containing 42 | 16.99 | 0.01123551 | NB |
| RP1-232L24.2 | | 16.31 | 0.024728355 | NB |
| IL13RA2 | interleukin 13 receptor, alpha 2 | 16.3 | 0.031216315 | NB |
| OR11H12 | olfactory receptor, family 11, subfamiy H, member 12 | 16.28 | 0.000660549 | NB |
| RP11-9H16.1 | | 16.25 | 0.021446576 | NB |
| RP11-670N15.2 | | 16.22 | 0.032630531 | NB |
| RP11-326E22.1 | | 16.08 | 0.006136197 | NB |
| ASS1P9 | argininosuccinate synthetase 1 pseudogene 9 | 15.74 | 0.009002927 | NB |
| RP11-310H4.5 | | 15.28 | 0.00216299 | NB |
| NXT1P1 | NTF2-like export factor 1 pseudogene 1 | 15.26 | 0.042179156 | NB |
| HMGN2P25 | high mobility group nucleosomal binding domain 2 pse [symbol] | 15 | 0.045821322 | NB |
| MAGEC2 | melanoma antigen family C, 2 | 14.91 | 0.024662434 | NB |
| RPS12P21 | ribosomal protein S12 pseudogene 21 | 14.9 | 0.038857039 | NB |
| HSPB3 | heat shock 27 kDa protein 3 | 14.68 | 0.011561372 | NB |
| HOXD11 | homeobox D11 | 14.62 | 0.002516383 | NB |
| ANKRD7 | ankyrin repeat domain 7 | 14.38 | 0.007839036 | NB |
| PSG1 | pregnancy specific beta-1-glycoprotein 1 | 14.26 | 0.014251282 | NB |
| GDNF | glial cell derived neurotrophic factor | 14.13 | 0.00731008 | NB |
| RP3-406A7.5 | | 14.13 | 0.047995343 | NB |
| PSG7 | pregnancy specific beta-1-glycoprotein 7 (gene/pseudo [symbol] | 14.09 | 0.025544289 | NB |
| RP3-432I18.1 | | 13.99 | 0.042179156 | NB |
| GAGE12D | G antigen 12D | 13.96 | 0.020177433 | NB |
| RPL7P56 | ribosomal protein L7 pseudogene 56 | 13.44 | 0.006122033 | NB |
| GTF2A1L | general transcription factor IIA, 1-like | 13.36 | 0.039174691 | NB |
| FBP2 | fructose-1,6-bisphosphatase 2 | 13.18 | 0.040365068 | NB |
| ACTBP8 | actin, beta pseudogene 8 | 13.05 | 0.006307706 | NB |
| MAGEA11 | melanoma antigen family A, 11 | 13.03 | 0.018744092 | NB |
| RSL24D1P2 | ribosomal L24 domain containing 1 pseudogene 2 | 13 | 0.035978626 | NB |
| OR2H5P | olfactory receptor, family 2, subfamily H, member 5 ps [symbol] | 12.88 | 0.029540253 | NB |
| AC002076.9 | | 12.77 | 0.024189646 | NB |
| C1QTNF9-AS1 | C1QTNF9 antisense RNA 1 | 12.72 | 0.011759771 | NB |
| OR52N2 | olfactory receptor, family 52, subfamily N, member 2 | 12.7 | 0.010901498 | NB |
| RP11-973N13.3 | | 12.53 | 0.002704784 | NB |
| ANKRD45 | ankyrin repeat domain 45 | 12.49 | 0.008813638 | NB |
| OR2T12 | olfactory receptor, family 2, subfamily T, member 12 | 12.45 | 0.048632504 | NB |
| PSG4 | pregnancy specific beta-1-glycoprotein 4 | 12.22 | 0.003256582 | NB |
| SPANXB2 | SPANX family, member B1 | 12.08 | 0.015009075 | NB |
| CTD-2206G10.1 | | 11.79 | 0.020435079 | NB |
| CHL1-AS2 | CHL1 antisense RNA 2 | 11.76 | 0.008620126 | NB |
| RP11-316E14.2 | | 11.63 | 0.042283368 | NB |
| PLAC1 | placenta-specific 1 | 11.62 | 0.032391329 | NB |
| MRPS17P9 | mitochondrial ribosomal protein S17 pseudogene 9 | 11.55 | 0.042179156 | NB |
| RP11-290F24.4 | | 7.84 | 0.025644286 | NB |
| AL359392.1 | | 7.83 | 0.047209651 | NB |
| BAI3 | | 7.82 | 0.008070194 | NB |
| ZEB2P1 | zinc finger E-box binding homeobox 2 pseudogene 1 | 7.78 | 0.010062094 | NB |
| CDK2AP2P1 | cycin-dependent kinase 2 associated protein 2 pseudo [symbol] | 7.74 | 0.048733769 | NB |
| AC105461.1 | | 7.72 | 0.049369302 | NB |
| RP11-138J23.1 | | 7.7 | 0.034551581 | NB |
| RP11-132G10.2 | | 7.68 | 0.034369721 | NB |
| SLC9C2 | solute carrier family 9, member C2 (putative) | 7.59 | 0.001888785 | NB |
| NELL1 | NEL-like 1 (chicken) | 7.59 | 0.028688235 | NB |
| RPS18P1 | ribosomal protein S18 pseudogene 1 | 7.59 | 0.04941826 | NB |
| RP11-1007G5.2 | | 7.49 | 0.041920281 | NB |
| OR9A3P | olfactory receptor, family 9, subfamily A, member 3 ps [symbol] | 7.42 | 0.0131522 | NB |
| SLCO1A2 | solute carrier organic anion transporter family, membe [symbol] | 7.28 | 0.038479039 | NB |
| GRIA2 | glutamate receptor, ionotropic, AMPA 2 | 7.27 | 0.029382731 | NB |
| METTL11B | methyltransferase like 11B | 7.19 | 0.042437855 | NB |
| MYLKP1 | myosin light chain kinase pseudogene 1 | 7.19 | 0.041397125 | NB |
| LINC00189 | long intergenic non-protein coding RNA 189 | 7.1 | 0.035553997 | NB |
| RP11-384F7.2 | | 7.07 | 0.017952081 | NB |
| MAGEA9B | melanoma antigen family A, 9B | 7.06 | 0.014094423 | NB |
| SRGAP3-AS1 | SRGAP3 antisense RNA 1 | 7.05 | 0.046077602 | NB |
| PSG9 | pregnancy specific beta-1-glycoprotein 9 | 7.05 | 0.030297821 | NB |
| RP11-959F10.4 | | 7.04 | 0.001238714 | NB |
| OR52E8 | olfactory receptor, family 52, subfamily E, member 8 | 6.97 | 0.042179156 | NB |
| RP11-1365D11.1 | | 6.93 | 0.041659897 | NB |
| AC018682.6 | | 6.92 | 0.018687432 | NB |
| HNRNPCL1 | heterogeneous nuclear ribonucleoprotein C-like 1 | 6.89 | 0.002516383 | NB |
| NLRP4 | NLR family, pyrin domain containing 4 | 6.84 | 0.010808151 | NB |
| LGALS12 | lectin, galactoside-binding, soluble, 12 | 6.78 | 0.037825665 | NB |
| DCAF4L2 | DDB1 and CUL4 associated factor 4-like 2 | 6.74 | 0.032737245 | NB |
| OR5K1 | olfactory receptor, family 5, subfamily K, member 1 | 6.67 | 0.007926133 | NB |
| AC079753.5 | | 6.63 | 0.048733769 | NB |
| TPTE2P6 | transmembrane phosphoinositide 3-phosphatase and t [symbol] | 6.62 | 0.003242319 | NB |
| LGI4 | leucine-rich repeat LGI family, member 4 | 6.57 | 0.005926234 | NB |
| KANK4 | KN motif and ankyrin repeat domains 4 | 6.55 | 0.041739925 | NB |
| TDPX2 | | 6.49 | 0.01692508 | NB |
| FPGT-TNNI3K | FPGT-TNNI3K readthrough | 6.48 | 0.001351513 | NB |
| RP11-206P5.2 | | 6.46 | 0.036254638 | NB |
| PI15 | peptidase inhibitor 15 | 6.46 | 0.011925792 | NB |
| LGI1 | leucine-rich, glioma inactivated 1 | 6.45 | 0.021949515 | NB |
| OPRD1 | opioid receptor, delta 1 | 6.42 | 0.044163971 | NB |
| TAAR6 | trace amine associated receptor 6 | 6.42 | 0.006632488 | NB |
| ISL2 | ISL LIM homeobox 2 | 6.42 | 0.009764943 | NB |
| OR7E91P | olfactory receptor, family 7, subfamily E, member 91 ps [symbol] | 6.41 | 0.036277389 | NB |
| ANKFN1 | ankyrin-repeat and fibronectin type III domain containi [symbol] | 6.41 | 0.006344984 | NB |
| UROC1 | urocanate hydratase 1 | 6.38 | 0.011561372 | NB |
| IGFN1 | immunoglobulin-like and fibronectin type III domain co [symbol] | 6.36 | 0.042370292 | NB |
| PRL | prolactin | 6.3 | 0.03495366 | NB |
| FRAS1 | Fraser syndrome 1 | 6.22 | 0.000149689 | NB |
| FSCN1 | fascin actin-bundling protein 1 | 6.21 | 0.000438786 | NB |
| FEM1AP4 | fem-1 homolog a (C. elegans) pseudogene 4 | 6.21 | 0.007806012 | NB |
| TERF1P1 | telomeric repeat binding factor (NIMA-interacting) 1 ps [symbol] | 6.21 | 0.043875977 | NB |
| RP11-54D18.2 | | 6.2 | 0.023106635 | NB |
| PCDHB3 | protocadherin beta 3 | 6.12 | 0.002421425 | NB |
| TOB2P1 | transducer of ERBB2, 2 pseudogene 1 | 6.06 | 0.024375591 | NB |
| OTOGL | otogelin-like | 6.06 | 0.018812935 | NB |
| C1orf185 | | 6.05 | 0.012843104 | NB |
| SYTL5 | synaptotagmin-like 5 | 11.47 | 0.014391143 | NB |
| RPS2P39 | ribosomal protein S2 pseudogene 39 | 11.44 | 0.025105736 | NB |
| CEACAMP5 | carcinoembryonic antigen-related cell adhesion molec [symbol] | 11.43 | 0.005369816 | NB |
| TFF2 | trefoil factor 2 | 11.43 | 0.008928345 | NB |
| AC016737.1 | | 11.35 | 0.007926133 | NB |
| RP11-438N16.2 | | 11.34 | 0.016120921 | NB |
| RP11-634B7.5 | | 11.31 | 0.038385003 | NB |
| RP11-108F13.2 | | 11.3 | 0.00239315 | NB |
| RP11-386I14.3 | | 11.25 | 0.015009075 | NB |
| RP11-885B4.1 | | 11.24 | 0.019586567 | NB |
| GRIN2B | glutamate receptor, ionotropic, N-methylD-aspartate [symbol] | 11.08 | 0.000583885 | NB |
| RPSAP43 | ribosomal protein SA pseudogene 43 | 11.05 | 0.04941826 | NB |
| OR2M2 | olfactory receptor, family 2, subfamily M, member 2 | 11.04 | 0.017682347 | NB |
| RP11-69I13.1 | | 10.87 | 0.011874924 | NB |
| RPS20P24 | ribosomal protein S20 pseudogene 24 | 10.66 | 0.025280278 | NB |
| CASP12 | caspase 12 (gene/pseudogene) | 10.62 | 0.00105356 | NB |
| AKAP6 | A kinase (PRKA) anchor protein 6 | 10.55 | 0.004785714 | NB |
| RP11-294J22.6 | | 10.54 | 0.049758592 | NB |
| RP1-273G13.2 | | 10.4 | 0.001483816 | NB |
| RP11-115C10.1 | | 10.28 | 0.014679168 | NB |
| OR2J3 | olfactory receptor, family 2, subfamily J, member 3 | 10.2 | 0.04941826 | NB |
| OR11H1 | olfactory receptor, family 11, subfamily H, member 1 | 10.15 | 0.014094423 | NB |
| OBP2A | odorant binding protein 2A | 10.14 | 0.038385003 | NB |
| GAGE13 | G antigen 13 | 10.07 | 0.048733769 | NB |
| SPINK13 | serine peptidase inhibitor, Kazal type 13 (putative) | 10 | 0.000900029 | NB |
| RP11-972K6.1 | | 10 | 0.046077602 | NB |
| KRT18P29 | keratin 18 pseudogene 29 | 9.86 | 0.001205418 | NB |
| RAET1L | retinoic acid early transcript 1L | 9.74 | 0.028546721 | NB |
| MYH1 | myosin, heavy chain 1, skeletal muscle, adult | 9.7 | 0.001681771 | NB |
| RP11-36B15.1 | | 9.49 | 0.047995343 | NB |
| RP11-889L3.4 | | 9.46 | 0.025105736 | NB |
| GLRA4 | glycine receptor, alpha 4 | 9.39 | 0.00250769 | NB |
| RP11-7G23.5 | | 9.37 | 0.041659897 | NB |
| AC008537.1 | | 9.36 | 0.00140516 | NB |
| AP002380.1 | | 9.3 | 0.009891102 | NB |
| RP3-461F17.2 | | 9.26 | 0.04756409 | NB |
| CR848007.6 | | 9.19 | 0.036211389 | NB |
| MYLK-AS1 | MYLK antisense RNA 1 | 9.17 | 0.029945212 | NB |
| MAGEB2 | melanoma antigen family B, 2 | 9.09 | 0.015914763 | NB |
| CFL1P2 | cofilin 1 (non-muscle) pseudogene 2 | 9.04 | 0.010626103 | NB |
| AC007557.1 | | 9.03 | 0.011581165 | NB |
| OR1D4 | olfactory receptor, family 1, subfamily D, member 4 (ge [symbol] | 8.93 | 0.017845056 | NB |
| MAGEC3 | melanoma antigen family C, 3 | 8.81 | 0.001071925 | NB |
| AC087491.2 | | 8.73 | 0.049369302 | NB |
| TTLL10-AS1 | TTLL10 antisense RNA 1 | 8.6 | 0.038385003 | NB |
| RP11-416N13.1 | | 8.46 | 0.04756409 | NB |
| AADAC | arylacetamide deacetylase | 8.36 | 0.028010999 | NB |
| RPL27AP8 | ribosomal protein L27a pseudogene 8 | 8.28 | 0.003936897 | NB |
| OR2M5 | olfactory receptor, family 2, subfamily M, member 5 | 8.21 | 0.019371037 | NB |
| FAM3D | family with sequence similarity 3, member D | 8.17 | 0.043875977 | NB |
| SLCO1B1 | solute carrier organic anion transporter family, membe [symbol] | 8.15 | 0.045588672 | NB |
| AC093110.3 | | 8.09 | 0.006633885 | NB |
| FUCA1P1 | fucosidase, alpha-L-1, tissue pseudogene 1 | 8.05 | 0.004515513 | NB |
| MAGEA8 | melanoma antigen family A, 8 | 8.05 | 0.029382731 | NB |
| RP11-557F20.2 | | 8 | 0.029762656 | NB |
| CIB4 | calcium and integrin binding family member 4 | 7.96 | 0.013029879 | NB |
| SFRP1 | secreted frizzled-related protein 1 | 7.93 | 0.018812935 | NB |
| RP11-315I20.3 | | 6.02 | 0.022360081 | NB |
| TRIM71 | tripartite motif containing 71, E3 ubiquitin protein ligas [symbol] | 5.97 | 0.045176166 | NB |
| CRB1 | crumbs family member 1, photoreceptor morphogenes [symbol] | 5.95 | 0.004290131 | NB |
| HPSE2 | heparanase 2 | 5.92 | 0.031391184 | NB |
| FSHR | follide stimulating hormone receptor | 5.86 | 0.020177433 | NB |
| RND2 | Rho family GTPase 2 | 5.86 | 0.003059735 | NB |
| VSX1 | visual system homeobox 1 | 5.86 | 0.003695654 | NB |
| GJB2 | gap junction protein, beta 2, 26 kDa | 5.85 | 0.001681771 | NB |
| LEFTY2 | left-right determination factor 2 | 5.8 | 0.030404894 | NB |
| PPP1R1C | protein phosphatase 1, regulatory (inhibitor) subunit 1 [symbol] | 5.8 | 0.000704181 | NB |
| ELOVL2 | ELOVL fatty acid elongase 2 | 5.74 | 0.02643572 | NB |
| AC107983.2 | | 5.71 | 0.008917729 | NB |
| RP13-210D15.1 | | 5.71 | 0.042179156 | NB |
| MPZ | myelin protein zero | 5.67 | 0.033656613 | NB |
| DNM3OS | DNM3 opposite strand/antisense RNA | 5.66 | 0.028426013 | NB |
| KLHL13 | kelch-like family member 13 | 5.65 | 0.031092971 | NB |
| HHATL | hedgehog acyltransferase-like | 5.64 | 0.0186415 | NB |
| RP11-351M16.1 | | 5.63 | 0.046077602 | NB |
| HCN1 | hyperpolarization activated cyclic nucleotide-gated pot [symbol] | 5.61 | 0.021040385 | NB |
| CEACAMP1 | carcinoembryonic antigen-related cell adhesion molec [symbol] | 5.61 | 0.036277389 | NB |
| RP11-536C10.24 | | 5.59 | 0.006136197 | NB |
| CITED4 | Cbp/p300-interacting transactivator, with Glu/Asp-rich | 5.58 | 0.007203049 | NB |
| AMDP1 | | 5.55 | 0.045085282 | NB |
| RP13-547K6.1 | | 5.53 | 0.010808151 | NB |
| C5orf27 | | 5.49 | 0.033515231 | NB |
| SLC22A10 | solute carrier family 22, member 10 | 5.42 | 0.008187016 | NB |
| TSPEAR | thrombospondin-type laminin G domain and EAR repea [symbol] | 5.42 | 0.007254752 | NB |
| U82695.9 | | 5.42 | 0.001227747 | NB |
| KIAA0087 | KIAA0087 | 5.4 | 0.000569695 | NB |
| MOCS1P1 | molybdenum cofactor synthesis 1 pseudogene 1 | 5.37 | 0.012166712 | NB |
| MYH2 | myosin, heavy chain 2, skeletal muscle, adult | 5.37 | 0.005926234 | NB |
| RP11-959F10.5 | | 5.36 | 0.00328693 | NB |
| SERPINA4 | serpin peptidase inhibitor, clade A (alpha-1 antiprotein [symbol] | 5.3 | 0.006480309 | NB |
| TPD52L1 | tumor protein D52-like 1 | 5.28 | 0.028688235 | NB |
| SLC30A8 | solute carrier family 30 (zinc transporter), member 8 | 5.27 | 0.034885381 | NB |
| RGS6 | regulator of G-protein signaling 6 | 5.25 | 0.016310813 | NB |
| DLX6-AS2 | DLX6 antisense RNA 2 | 5.24 | 0.045588672 | NB |
| C9orf153 | | 5.24 | 0.009764943 | NB |
| MYH13 | myosin, heavy chain 13, sleletal muscle | 5.24 | 0.002724323 | NB |
| CA10 | carbonic anhydrase X | 5.24 | 0.006122033 | NB |
| DGKB | diacylglycerol kinase, beta 90 kDa | 5.23 | 0.012651023 | NB |
| GABRB1 | gamma-aminobutyric acid (GABA) A receptor, beta 1 | 5.22 | 0.020435079 | NB |
| AC013268.3 | | 5.2 | 0.009041786 | NB |
| RP11-696L21.1 | | 5.18 | 0.026859197 | NB |
| RP11-450I19.2 | | 5.15 | 0.010626103 | NB |
| TRIM9 | tripartite motif containing 9 | 5.12 | 0.022360081 | NB |
| GFRA3 | GDNIF family receptor alpha 3 | 5.11 | 0.015027152 | NB |
| XIRP2 | xin actin-binding repeat containing 2 | 5.1 | 0.009662367 | NB |
| RHO | rhodopsin | 5.1 | 0.029762656 | NB |
| CLDN1 | claudin 1 | 5.09 | 0.022360081 | NB |
| IRGC | immunity-related GTPase family, cinema | 5.09 | 0.016120921 | NB |
| PCK1 | phosphoenolpyruvate carboxykinase 1 (soluble) | 5.04 | 0.023851247 | NB |
| MAPK10 | mitogen-activated protein kinase 10 | 5.03 | 0.024328524 | NB |
| EPHA6 | EPH receptor A6 | 5.02 | 0.010347516 | NB |
| NXF2B | nuclear RNA export factor 2B | 5.02 | 0.026302018 | NB |
| HMGA2 | high mobility group AT-hook 2 | 5 | 0.045638568 | NB |
| KLK13 | kallikrein-related peptidase 13 | 5 | 0.010808151 | NB |
| RP1-14D6.2 | | 5 | 0.014679168 | NB |
| C21orf90 | | 4.93 | 0.008608265 | NB |
| RP11-678B3.1 | | 4.91 | 0.007605535 | NB |
| CHRNA1 | cholinergic receptor, nicotinic, alpha 1 (muscle) | 4.9 | 0.02052367 | NB |
| RP11-501I19.4 | | 4.88 | 0.027988697 | NB |
| GABRR1 | gamma-aminobutyric acid (GABA) A receptor, rho 1 | 4.88 | 0.003640543 | NB |
| DKK3 | dickkopf WNT signaling pathway inhibitor 3 | 4.87 | 0.003059735 | NB |
| RP11-1236K1.8 | | 4.86 | 0.00729215 | NB |
| CHRNA9 | cholinergic receptor, nicotinic, alpha 9 (neuronal) | 4.82 | 0.045821322 | NB |
| TSPEAR-AS1 | TSPEAR antisense RNA 1 | 4.82 | 0.022558878 | NB |
| NBEAP3 | neurobeachin pseudogene 3 | 4.81 | 0.003673452 | NB |
| AC093162.3 | | 4.79 | 0.023511088 | NB |
| LRRC37A11P | leucine rich repeat containing 37, member A11, pseud [symbol] | 4.77 | 0.002176072 | NB |
| RP11-318M2.2 | | 4.76 | 0.004790916 | NB |
| RP11-760D2.10 | | 4.75 | 0.025811031 | NB |
| MGP | matrix Gla protein | 4.73 | 0.008917729 | NB |
| MYH8 | myosin, heavy chain 8, skeletal muscle, perinatal | 4.73 | 0.004290131 | NB |
| RFPL4B | ret finger protein-like 4B | 4.7 | 0.020204306 | NB |
| CTD-2314B22.2 | | 4.69 | 0.008917729 | NB |
| RP11-439H8.4 | | 4.67 | 0.010924889 | NB |
| RP11-72K17.1 | | 4.66 | 0.014141242 | NB |
| MYO5B | myosin VB | 4.66 | 0.031092971 | NB |
| CAPN9 | calpain 9 | 4.63 | 0.013235921 | NB |
| OR51B6 | olfactory receptor, family 51, subfamily B, member 6 | 4.63 | 0.007839036 | NB |
| PRIMA1 | proline rich membrane anchor 1 | 4.63 | 0.006390779 | NB |
| DNAJC12 | DnaJ (Hsp40) homolog, subfamily C, member 12 | 4.62 | 0.005329798 | NB |
| LRRC14B | leucine rich repeat containing 14B | 4.61 | 0.047474072 | NB |
| AGR2 | anterior gradient 2 | 4.59 | 0.032737245 | NB |
| EGF | epidermal growth factor | 4.58 | 0.013100955 | NB |
| KRT8P19 | keratin 8 pseudogene 19 | 4.57 | 0.015640563 | NB |
| FLRT3 | fibronectin leucine rich transmembrane protein 3 | 4.55 | 0.00657893 | NB |
| AC005009.2 | | 4.53 | 0.017521647 | NB |
| MYH7 | myosin, heavy chain 7, cardiac muscle, beta | 4.53 | 0.016408594 | NB |
| RP11-106M7.4 | | 4.5 | 0.003500427 | NB |
| NDP | Norrie disease (pseudoglioma) | 4.5 | 0.029382731 | NB |
| LINC00086 | | 4.48 | 0.016398129 | NB |
| SV2A | synaptic vesicle glycoprotein 2A | 4.47 | 0.024328524 | NB |
| TEKT4 | tektin 4 | 4.46 | 0.004092549 | NB |
| STRA8 | stimulated by retinoic acid 8 | 4.44 | 0.015103445 | NB |
| KCNIP1 | Kv channel interacting protein 1 | 4.43 | 0.018812935 | NB |
| RP11-373N22.4 | | 4.41 | 0.025869773 | NB |
| SERPINA3 | serpin peptidase inhibitor, clade A (alpha-1 anti protein [symbol] | 4.41 | 0.042370292 | NB |
| HMGB3P2 | high mobility group box 3 pseudogene 2 | 4.41 | 0.01102242 | NB |
| MEGF10 | multiple EGF-like-domains 10 | 4.4 | 0.005329798 | NB |
| RP11-342H21.2 | | 4.39 | 0.047474072 | NB |
| C1QTNF3 | C1q and tumor necrosis factor related protein 3 | 4.38 | 0.001483816 | NB |
| SYNDIG1L | synapse differentiation inducing 1-like | 4.36 | 0.020204306 | NB |
| USP17L2 | ubiquitin specific peptidase 17-like family member 2 | 4.35 | 0.039288394 | NB |
| RP11-81K13.1 | | 4.35 | 0.019520722 | NB |
| RP11-187E13.2 | | 4.35 | 0.036367629 | NB |
| C12orf56 | | 4.33 | 0.02678915 | NB |
| HNF4A | hepatocyte nuclear factor 4, alpha | 4.32 | 0.023187328 | NB |
| XXbac-B33L19.3 | | 4.32 | 0.028569961 | NB |
| IGLON5 | IgLON family member 5 | 4.3 | 0.028071732 | NB |
| HTR3B | 5-hyd roxytrypta mine (serotonin) receptor 3B, ionotrop [symbol] | 4.29 | 0.049216823 | NB |
| RP11-146E13.2 | | 4.28 | 0.002988917 | NB |
| RP11-160H12.3 | | 4.27 | 0.019371037 | NB |
| TMPRSS11F | transmembrane protease, serine 11F | 4.26 | 0.016219299 | NB |
| IGFBP2 | insulin-like growth factor binding protein 2, 36 kDa | 4.25 | 0.001148356 | NB |
| MIR584 | microRNA 584 | 4.25 | 0.033545013 | NB |
| GPR158 | G protein-coupled receptor 158 | 4.24 | 0.017221389 | NB |
| ZNF334 | zinc finger protein 334 | 4.24 | 0.049100171 | NB |
| Y_RNA | | 4.23 | 0.026859197 | NB |
| FREM1 | FRAS1 related extracellular matrix 1 | 4.22 | 0.014371388 | NB |
| CNDP1 | carnosine dipeptidase 1 (metallopeptidase M20 family) | 4.21 | 0.045638568 | NB |
| NR1H4 | nuclear receptor subfamily 1, group H, member 4 | 4.2 | 0.014367798 | NB |
| CXorf49 | | 4.2 | 0.04941826 | NB |
| MRGPRX3 | MAS-related GPR, member X3 | 4.18 | 0.007254752 | NB |
| SLC22A24 | solute carrier family 22, member 24 | 4.18 | 0.032272104 | NB |
| RBPJP5 | RBPJ pseudogene 5 | 4.15 | 0.042283368 | NB |
| SSX6 | synovial sarcoma, X breakpoint 6 (pseudogene) | 4.13 | 0.004509166 | NB |
| PNMAL1 | paraneoplastic Ma antigen famiy-like 1 | 4.12 | 0.003839527 | NB |
| CTB-35F21.4 | | 4.11 | 0.043653892 | NB |
| CNN2P2 | calponin 2 pseudogene 2 | 4.09 | 0.025644286 | NB |
| SALL1 | spat-like transcription factor 1 | 4.09 | 0.049100171 | NB |
| PCSK1 | proprotein convertase subtilisin/kexin type 1 | 4.08 | 0.003059735 | NB |
| PLA2G1B | phospholipase A2, group IB (pancreas) | 4.08 | 0.028780815 | NB |
| RP3-525N14.2 | | 4.08 | 0.012492516 | NB |
| PHBP13 | prohibitin pseudogene 13 | 4.07 | 0.049633437 | NB |
| PATE1 | prostate and testis expressed 1 | 4.04 | 0.017521647 | NB |
| SORCS2 | sortilin-related VPS10 domain containing receptor 2 | 4.03 | 0.039288394 | NB |
| EPS8L3 | EPS8-like 3 | 4.02 | 0.043875977 | NB |
| SLC22A6 | solute carrier famiy 22 (organic anion transporter), me [symbol] | 4.02 | 0.029762656 | NB |
| AC009237.9 | | 4 | 0.026579731 | NB |
| CER1 | cerberus 1, DAN family BMP antagonist | 4 | 0.033575171 | NB |
| RPL17P26 | ribosomal protein L17 pseudogene 26 | 3.99 | 0.006656541 | NB |
| HOXD13 | homeobox D13 | 3.98 | 0.00657893 | NB |
| RP5-886K2.1 | | 3.96 | 0.008079629 | NB |
| CCDC75P1 | coiled-coil domain containing 75 pseudogene 1 | 3.96 | 0.049851912 | NB |
| EMILIN1 | elastin microfibril interfacer 1 | 3.95 | 0.011925792 | NB |
| SULT1C3 | sulfotransferase family, cytosolic, 1C, member 3 | 3.95 | 0.031076174 | NB |
| PNPLA1 | patatin-like phospholipase domain containing 1 | 3.95 | 0.006706195 | NB |
| RP11-803B1.1 | | 3.94 | 0.008184312 | NB |
| OTOL1 | otolin 1 | 3.94 | 0.033214335 | NB |
| USP17L7 | ubiquitin specific peptidase 17-like family member 7 | 3.93 | 0.031092971 | NB |
| CABP4 | calcium binding protein 4 | 3.93 | 0.033656613 | NB |
| RP11-93I21.1 | | 3.92 | 0.033351877 | NB |
| RP11-732A19.6 | | 3.92 | 0.003430441 | NB |
| TNMD | tenomodulin | 3.92 | 0.02857165 | NB |
| SLC35D3 | solute carrier family 35, member D3 | 3.91 | 0.001964064 | NB |
| KB-1980E6.3 | | 3.91 | 0.006947328 | NB |
| CSMD3 | CUB and Sushi multiple domains 3 | 3.89 | 0.00175507 | NB |
| RP11-138E16.2 | | 3.89 | 0.00984494 | NB |
| RP11-281O15.4 | | 3.87 | 0.019304276 | NB |
| MMP16 | matrix metallopeptidase 16 (membrane-inserted) | 3.87 | 0.010840451 | NB |
| MYOG | myogenin (myogenic factor 4) | 3.86 | 0.014445675 | NB |
| KCNK16 | potassium channel, subfamily K, member 16 | 3.86 | 0.012777747 | NB |
| GJE1 | gap junction protein, epsilon 1, 23 kDa | 3.85 | 0.030508943 | NB |
| OR51K1P | olfactory receptor, family 51, subfamily K, member 1 p [symbol] | 3.85 | 0.004668469 | NB |
| TNN | tenascin N | 3.84 | 0.002421425 | NB |
| CFI | complement factor I | 3.84 | 0.002724323 | NB |
| GRIA4 | glutamate receptor, ionotropic, AMPA 4 | 3.83 | 0.004282816 | NB |
| CTD-3049M7.1 | | 3.83 | 0.028374775 | NB |
| RP5-1022P6.6 | | 3.83 | 0.025822313 | NB |
| UBXN10 | UBX domain protein 10 | 3.82 | 0.033880884 | NB |
| SPZ1 | spermatogenic leucine zipper 1 | 3.82 | 0.019575576 | NB |
| GJD4 | gap junction protein, delta 4, 40.1 kDa | 3.82 | 0.035979007 | NB |
| ALLC | allantoicase | 3.8 | 0.029299564 | NB |
| CRP | C-reactive protein, pentraxin-related | 3.79 | 0.030546079 | NB |
| SSPO | SCO-spondin | 3.78 | 0.049100171 | NB |
| ARTN | artemin | 3.77 | 0.024375591 | NB |
| RP11-15J10.3 | | 3.77 | 0.045296878 | NB |
| THSD4 | thrombospondin, type I, domain containing 4 | 3.77 | 0.017221389 | NB |
| TRPM3 | transient receptor potential cation channel, subfamily [symbol] | 3.76 | 0.036386177 | NB |
| RBBP4P4 | retinoblastoma binding protein 4 pseudogene 4 | 3.74 | 0.043902532 | NB |
| RP11-451K18.7 | | 3.74 | 0.049851912 | NB |
| EDDM3B | epididymal protein 3B | 3.74 | 0.027138346 | NB |
| SNORA27 | small nucleolar RNA, H/ACA box 27 | 3.73 | 0.043653892 | NB |
| BPIFB1 | BPI fold containing family B, member 1 | 3.73 | 0.031990541 | NB |
| OLFM3 | olfactomedin 3 | 3.72 | 0.008620126 | NB |
| USP17L6P | ubiquitin specific peptidase 17-like family member 6, p [symbol] | 3.72 | 0.005926234 | NB |
| GABRG2 | gamma-aminobutyric acid (GABA) A receptor, gamma [symbol] | 3.72 | 0.011960052 | NB |
| AL137067.1 | | 3.72 | 0.02857165 | NB |
| KRT8P11 | keratin 8 pseudogene 11 | 3.72 | 0.02857165 | NB |
| TEKT2 | tektin 2 (testicular) | 3.71 | 0.005760347 | NB |
| RNASE3 | ribonuclease, RNase A family, 3 | 3.71 | 0.014391143 | NB |
| GPC4 | glypican 4 | 3.71 | 0.039288394 | NB |
| KLF17 | Kruppel-like factor 17 | 3.7 | 0.009594634 | NB |
| CNGB3 | cyclic nucleotide gated channel beta 3 | 3.69 | 0.004030313 | NB |
| AF165138.7 | | 3.69 | 0.041042494 | NB |
| EFHC2 | EF-hand domain (C-terminal) containing 2 | 3.69 | 0.042306582 | NB |
| GAP43 | growth associated protein 43 | 3.68 | 0.012569126 | NB |
| RETN | resistin | 3.68 | 0.040365068 | NB |
| LIMCH1 | LIM and calponin homology domains 1 | 3.67 | 0.033656613 | NB |
| AC005754.1 | | 3.67 | 0.049100171 | NB |
| CTCFL | CCCTC-binding factor (zinc finger protein)-like | 3.67 | 0.010416999 | NB |
| ADCY5 | adenylate cyclase 5 | 3.66 | 0.036386177 | NB |
| BMPER | BMP binding endothelial regulator | 3.66 | 0.010840451 | NB |
| RP11-1081K18.1 | | 3.66 | 0.030404894 | NB |
| IGFBP3 | insulin-like growth factor binding protein 3 | 3.65 | 0.001483816 | NB |
| CYP7B1 | cytochrome P450, family 7, subfamily B, polypeptide 1 | 3.65 | 0.002148321 | NB |
| RP11-432I13.1 | | 3.63 | 0.007302655 | NB |
| CTD-2206G10.2 | | 3.62 | 0.027138346 | NB |
| CACNA1S | calcium channel, voltage-dependent, L type, alpha 1S s [symbol] | 3.61 | 0.014762458 | NB |
| PDGFA | platelet-derived growth factor alpha polypeptide | 3.61 | 0.001306624 | NB |
| BPI | bactericidal/permeability-increasing protein | 3.61 | 0.004949546 | NB |
| GPR87 | G protein-coupled receptor 87 | 3.6 | 0.034369721 | NB |
| WNK4 | WNK lysine deficient protein kinase 4 | 3.6 | 0.031092971 | NB |
| RP11-791G16.2 | | 3.59 | 0.047168612 | NB |
| CYP2C9 | cytochrome P450, family 2, subfamily C, polypeptide 9 | 3.57 | 0.033515231 | NB |
| SRP68P2 | signal recognition particle 68 kDa pseudogene 2 | 3.57 | 0.01262029 | NB |
| ASIC5 | acid-sensing (proton-gated) ion channel family membe [symbol] | 3.56 | 0.005369816 | NB |
| SLC18A1 | solute carrier family 18 (vesicular monoamine transpor [symbol] | 3.56 | 0.006479884 | NB |
| OR51M1 | olfactory receptor, family 51, subfamily M, member 1 | 3.55 | 0.00713094 | NB |
| LA16c-60H5.7 | | 3.55 | 0.005926234 | NB |
| KCNG3 | potassium voltage-gated channel, subfamily G, membe [symbol] | 3.54 | 0.036500639 | NB |
| GNGT1 | guanine nucleotide binding protein (G protein), gamma [symbol] | 3.53 | 0.042437855 | NB |
| IAPP | islet amyloid polypeptide | 3.53 | 0.016120921 | NB |
| ATP10B | ATPase, class V, type 10B | 3.52 | 0.031092971 | NB |
| ATP1B2 | ATPase, Na+/K+ transporting, beta 2 polypeptide | 3.51 | 0.028688235 | NB |
| FOXE3 | forkhead box E3 | 3.5 | 0.014251282 | NB |
| ANKRD20A4 | ankyrin repeat domain 20 family, member A4 | 3.5 | 0.015742933 | NB |
| RP11-496I2.2 | | 3.5 | 0.019899858 | NB |
| HOTAIRM1 | HOXA transcript antisense RNA, myeloid-specific 1 | 3.49 | 0.009381276 | NB |
| ZNF806 | zinc finger protein 806 | 3.47 | 0.024662434 | NB |
| HIST1H1T | histone cluster 1, H1t | 3.47 | 0.010509409 | NB |
| TSPAN5 | tetraspanin 5 | 3.46 | 0.003059735 | NB |
| RP11-586K2.1 | | 3.46 | 0.017221389 | NB |
| FAM75C2 | SPATA31 subfamily C, member 2 | 3.46 | 0.028688235 | NB |
| RP11-181D18.2 | | 3.45 | 0.024790121 | NB |
| SETP9 | SET pseudogene 9 | 3.44 | 0.031216315 | NB |
| CYP27C1 | cytochrome P450, family 27, subfamily C, polypeptide [symbol] | 3.44 | 0.011925792 | NB |
| SLC4A9 | solute carrier famiy 4, sodium bicarbonate cotransport [symbol] | 3.43 | 0.000583885 | NB |
| SMAD9 | SMAD family member 9 | 3.4 | 0.02643572 | NB |
| GAL3ST1 | galactose-3-O-sulfotransferase 1 | 3.4 | 0.03791799 | NB |
| MAGEB17 | melanoma antigen family B, 17 | 3.4 | 0.034334741 | NB |
| NKX3-2 | NK3 homeobox 2 | 3.39 | 0.016371906 | NB |
| PEX5L | peroxisomal biogenesis factor 5-like | 3.38 | 0.031092971 | NB |
| OR51J1 | olfactory receptor, family 51, subfamily J, member 1 (g [symbol] | 3.38 | 0.036189278 | NB |
| PTCHD2 | patched domain containing 2 | 3.37 | 0.00731008 | NB |
| TMPRSS12 | transmembrane (C-terminal) protease, serine 12 | 3.37 | 0.022558878 | NB |
| CORO2B | coronin, actin binding protein, 2B | 3.37 | 0.008917729 | NB |
| NINL | ninein-like | 3.37 | 0.005926234 | NB |
| PHBP5 | prohibitin pseudogene 5 | 3.36 | 0.009175608 | NB |
| SDAD1P2 | SDA1 domain containing 1 pseudogene 2 | 3.36 | 0.028426013 | NB |
| RP11-645N11.2 | | 3.35 | 0.000869748 | NB |
| SLC22A17 | solute carrier family 22, member 17 | 3.35 | 0.003839527 | NB |
| FAM106A | family with sequence similarity 106, member A | 3.35 | 0.014371388 | NB |
| SOX9 | SRY (sex determining region Y)-box 9 | 3.35 | 0.042370292 | NB |
| AKR1B1P1 | aldehyde reductase family 1, member B1 pseudogene [symbol] | 3.34 | 0.049100171 | NB |
| ABCC8 | ATP-binding cassette, sub-family C (CFTR/MRP), memb [symbol] | 3.34 | 0.023867249 | NB |
| ZNF541 | zinc finger protein 541 | 3.34 | 0.015742933 | NB |
| RP11-136I13.1 | | 3.33 | 0.017952081 | NB |
| RP11-732A19.5 | | 3.33 | 0.028688235 | NB |
| CLEC1A | C-type lectin domain family 1, member A | 3.33 | 0.005926234 | NB |
| NRXN1 | neurexin 1 | 3.32 | 0.042306582 | NB |
| MIR581 | microRNA 581 | 3.32 | 0.012651023 | NB |
| DLEU7 | deleted in lymphocytic leukemia, 7 | 3.32 | 0.019413544 | NB |
| RP11-355N15.1 | | 3.32 | 0.045518278 | NB |
| RP11-111F5.2 | | 3.31 | 0.019674294 | NB |
| KCNC2 | potassium voltage-gated channel, Shaw-related subfam [symbol] | 3.31 | 0.0064423 | NB |
| AMHR2 | anti-Mullerian hormone receptor, type II | 3.3 | 0.002397768 | NB |
| SLC2A12 | solute carrier family 2 (facilitated glucose transporter), [symbol] | 3.28 | 0.004495465 | NB |
| C9orf170 | | 3.28 | 0.01142727 | NB |
| AC012531.25 | | 3.28 | 0.01659065 | NB |
| LIPI | lipase, member I | 3.28 | 0.035096859 | NB |
| PCDHGA12 | protocadherin gamma subfamily A, 12 | 3.27 | 0.049100171 | NB |
| MIPEPP1 | mitochondrial intermediate peplidase pseudogene 1 | 3.27 | 0.02019187 | NB |
| PPP4R4 | protein phosphatase 4, regulatory subunit 4 | 3.27 | 0.046776361 | NB |
| GOLGA6L11P | golgin A6 family-like 11, pseudogene | 3.27 | 0.000599951 | NB |
| AC000110.1 | | 3.26 | 0.016753819 | NB |
| CHRM4 | cholinergic receptor, muscarinic 4 | 3.26 | 0.011041252 | NB |
| BX004987.3 | | 3.24 | 0.049638568 | NB |
| RP11-744D14.1 | | 3.24 | 0.003003823 | NB |
| CSRP3 | cysteine and glycine-rich protein 3 (cardiac LIM protein [symbol] | 3.23 | 0.009241778 | NB |
| NPAS3 | neuronal PAS domain protein 3 | 3.22 | 0.00657893 | NB |
| WNT3 | wingless type MMTV integration site family, member 3 | 3.22 | 0.013811098 | NB |
| RP11-496I2.5 | | 3.2 | 0.010840451 | NB |
| RP3-352A20.1 | | 3.19 | 0.002176072 | NB |
| SYT1 | synaptotagmin I | 3.19 | 0.036500639 | NB |
| SEC14L4 | SEC14-like 4 (S. cerevisiae) | 3.19 | 0.047474072 | NB |
| CELA2A | chymotrypsin-like elastase family, member 2A | 3.18 | 0.016408594 | NB |
| CTD-2158P22.1 | | 3.18 | 0.023867249 | NB |
| PDE6A | phosphodiesterase 6A, cGMP-specific, rod, alpha | 3.18 | 0.007320475 | NB |
| TBX3 | T-box 3 | 3.18 | 0.001007228 | NB |
| ABCA8 | ATP-binding cassette, sub-family A (ABC1), member 8 | 3.18 | 0.049100171 | NB |
| PCDHB2 | protocadherin beta 2 | 3.16 | 0.008917729 | NB |
| DRD2 | dopamine receptor D2 | 3.16 | 0.019111626 | NB |
| ALS2CR11 | amyotrophic lateral sclerosis 2 (juvenile) chromosome | 3.15 | 0.02052367 | NB |
| RP11-510H23.1 | | 3.15 | 0.017138695 | NB |
| FAM194B | glutamate-rich 6B | 3.15 | 0.045518278 | NB |
| KCNJ3 | potassium inwardly-rectifying channel, subfamily J, me [symbol] | 3.13 | 0.002347701 | NB |
| DZIP1 | DAZ interacting zinc finger protein 1 | 3.12 | 0.036386177 | NB |
| HIF3A | hypoxia inducible factor 3, alpha subunit | 3.12 | 0.024328524 | NB |
| SLC17A3 | solute carrier family 17 (organic anion transporter), me [symbol] | 3.11 | 0.038991906 | NB |
| RP11-1134I14.2 | | 3.11 | 0.019371037 | NB |
| RP11-1396O13.8 | | 3.1 | 0.048226887 | NB |
| RP11-307I2.1 | | 3.1 | 0.009325595 | NB |
| RP11-114H24.5 | | 3.1 | 0.039288394 | NB |
| AC020907.2 | | 3.1 | 0.047474072 | NB |
| AC107021.1 | | 3.09 | 0.025748667 | NB |
| GYPE | glycophorin E (MNS blood group) | 3.09 | 0.02287369 | NB |
| SALL4 | spalt-like transcription factor 4 | 3.09 | 0.033656613 | NB |
| SPAG17 | sperm associated antigen 17 | 3.08 | 0.009839457 | NB |
| AFF3 | AF4/FMR2 family, member 3 | 3.07 | 0.039288394 | NB |
| TDGF1 | teratocarcinoma-derived growth factor 1 | 3.07 | 0.049369302 | NB |
| STK19P | | 3.07 | 0.000676183 | NB |
| SFTPA1 | surfactant protein A1 | 3.06 | 0.033575171 | NB |
| SNAI1 | snail family zinc finger 1 | 3.06 | 0.00657893 | NB |
| AC092155.2 | | 3.05 | 0.047995343 | NB |
| RAPGEF4 | Rap guanine nucleotide exchange factor (GEF) 4 | 3.05 | 0.02052367 | NB |
| AKR1B15 | aldo-keto reductase family 1, member B15 | 3.05 | 0.033277256 | NB |
| ADAMTS7 | ADAM metallopeptidase with thrombospondin type 1 r [symbol] | 3.05 | 0.008917729 | NB |
| COLEC12 | collectin sub-family member 12 | 3.05 | 0.045638568 | NB |
| PTGS2 | prostaglandin-endoperoxide synthase 2 (prostaglandin [symbol] | 3.04 | 0.039288394 | NB |
| ULBP3 | UL16 binding protein 3 | 3.04 | 0.010509409 | NB |
| HMGB3P31 | high mobility group box 3 pseudogene 31 | 3.04 | 0.020318802 | NB |
| AQP7P1 | aquaporin 7 pseudogene 1 | 3.03 | 0.039288394 | NB |
| ABCC6P2 | ATP-binding cassette, sub-family C, member 6 pseudog [symbol] | 3.03 | 0.039234101 | NB |
| ZNF331 | zinc finger protein 331 | 3.03 | 7.63E−05 | NB |
| KY | kyphoscoliosis peptidase | 3.02 | 0.04213223 | NB |
| GS1-184P14.1 | | 3.01 | 0.01305666 | NB |
| DMD | dystrophin | 3 | 0.02643572 | NB |
| AC079354.1 | | 2.99 | 0.000590409 | NB |
| MS4A3 | membrane-spanning 4-domains, subfamily A, member [symbol] | 2.99 | 0.021446576 | NB |
| C14orf57 | | 2.99 | 0.039234101 | NB |
| C14orf132 | | 2.99 | 0.033656613 | NB |
| MSTN | myostatin | 2.98 | 0.021083625 | NB |
| AL772307.1 | | 2.98 | 0.016310813 | NB |
| PIEZO2 | piezo-type mechanosensitive ion channel component 2 | 2.98 | 0.013100955 | NB |
| ZNF355P | zinc finger protein 355, pseudogene | 2.98 | 0.010441837 | NB |
| KLK14 | kallikrein-related peptidase 14 | 2.97 | 0.021373132 | NB |
| MACROD2 | MACRO domain containing 2 | 2.97 | 0.024328524 | NB |
| RP11-521J5.1 | | 2.96 | 0.031092971 | NB |
| TLX2 | T-cell leukemia homeobox 2 | 2.95 | 0.04488914 | NB |
| RPL7AP26 | ribosomal protein L7a pseudogene 25 | 2.95 | 0.036500639 | NB |
| MOXD1 | monooxygenase, DBH-like 1 | 2.95 | 0.02643572 | NB |
| RP5-1198O20.4 | | 2.94 | 0.027941793 | NB |
| RP11-190P13.1 | | 2.94 | 0.025784335 | NB |
| SOSTDC1 | sclerostin domain containing 1 | 2.94 | 0.02875596 | NB |
| HCAR1 | hydroxycarboxylic acid receptor 1 | 2.94 | 0.015249492 | NB |
| TCP11 | t-complex 11, testis-specific | 2.93 | 0.012073496 | NB |
| FXYD1 | FXYD domain containing ion transport regulator 1 | 2.93 | 0.022360081 | NB |
| C7orf61 | | 2.92 | 0.012651023 | NB |
| DDX25 | DEAD (Asp-Glu-Ala-Asp) box helicase 25 | 2.92 | 0.035185369 | NB |
| OXTR | oxytocin receptor | 2.91 | 0.011547361 | NB |
| FAM75A1 | SPATA31 subfamily A, member 1 | 2.91 | 0.012492516 | NB |
| AC016251.1 | | 2.91 | 0.012118209 | NB |
| AC008079.9 | | 2.91 | 0.039259375 | NB |
| MIR548I1 | microRNA 548i-1 | 2.89 | 0.039934696 | NB |
| RP11-510H23.3 | | 2.89 | 0.019413544 | NB |
| RPS20P22 | ribosomal protein S20 pseudogene 22 | 2.89 | 0.035096859 | NB |
| C1orf173 | | 2.88 | 0.03877208 | NB |
| HIGD1AP9 | HIG1 hypoxia inducible domain family, member 1A pse [symbol] | 2.88 | 0.008789839 | NB |
| ST13P12 | suppression of tumorigenicity 13 (colon carcinoma) (Hs [symbol] | 2.87 | 0.0049729 | NB |
| RP11-15J10.8 | | 2.87 | 0.020318802 | NB |
| DCHS1 | dachsous cadherin-related 1 | 2.87 | 0.02643572 | NB |
| TAF7L | TAF7-like RNA polymerase II, TATA box binding protein | 2.87 | 0.027941793 | NB |
| RP11-1286E23.6 | | 2.86 | 0.036386177 | NB |
| RP11-798K3.4 | | 2.86 | 0.031076174 | NB |
| AC073264.10 | | 2.85 | 0.032737245 | NB |
| CMYA5 | cardiomyopathy associated 5 | 2.84 | 0.018812935 | NB |
| HOXA3 | homeobox A3 | 2.84 | 0.033656613 | NB |
| RP11-812E19.6 | | 2.83 | 0.015742933 | NB |
| UBE2U | ubiquitin-conjugating enzyme E2U (putative) | 2.82 | 0.025644286 | NB |
| RP11-277P12.20 | | 2.82 | 0.019058997 | NB |
| CBLN4 | cerebellin 4 precursor | 2.82 | 0.026146007 | NB |
| RP11-146D12.2 | | 2.81 | 0.042370292 | NB |
| PRSS55 | protease, serine, 55 | 2.8 | 0.028426013 | NB |
| RTDR1 | | 2.8 | 0.033575171 | NB |
| EPHA2 | EPH receptor 42 | 2.78 | 0.014371388 | NB |
| ULBP2 | UL16 binding protein 2 | 2.78 | 0.036386177 | NB |
| SPEM1 | spermatid maturation 1 | 2.78 | 0.023187328 | NB |
| KRT20 | keratin 20 | 2.78 | 0.012799782 | NB |
| RP11-325P15.1 | | 2.77 | 0.031092971 | NB |
| LONRF2 | LON peptidase N-terminal domain and ring finger 2 | 2.77 | 0.022360081 | NB |
| CDH10 | cadherin 10, type 2 (T2-cadherin) | 2.77 | 0.016475123 | NB |
| SORCS3 | sortilin-related VPS10 domain containing receptor 3 | 2.77 | 0.003476545 | NB |
| ARHGAP40 | Rho GTPase activating protein 40 | 2.77 | 0.00654708 | NB |
| HOXA2 | homeobox A2 | 2.76 | 0.013100955 | NB |
| C12orf50 | | 2.76 | 0.014949579 | NB |
| FAM19A1 | family with sequence similarity 19 (chemokine (C-C mo [symbol] | 2.75 | 0.019074797 | NB |
| RP11-695J4.2 | | 2.75 | 0.017818411 | NB |
| CTD-2201I18.1 | uncharacterized LOC101929215 | 2.74 | 0.02643572 | NB |
| CLDN2 | claudin 2 | 2.74 | 0.016310813 | NB |
| PRRX1 | paired related homeobox 1 | 2.73 | 0.042370292 | NB |
| FAM46A | family with sequence similarity 46, member A | 2.73 | 0.003059735 | NB |
| CACNA2D4 | calcium channel, voltage-dependent, alpha 2/delta sub [symbol] | 2.73 | 0.049100171 | NB |
| RP11-400L8.2 | | 2.72 | 0.017559073 | NB |
| ASS1P7 | argininosurxinate synthetase 1 pseudogene 7 | 2.72 | 0.024493172 | NB |
| NDST4 | N-deacetylase/N-sulfatransferase (heparan glucosamin [symbol] | 2.72 | 0.03781544 | NB |
| KB-1554H10.1 | | 2.72 | 0.019058997 | NB |
| NYNRIN | NYN domain and retroviral integrase containing | 2.72 | 0.036386177 | NB |
| CADM4 | cell adhesion molecule 4 | 2.72 | 0.008070194 | NB |
| GABRB2 | gamma-aminobutyric acid (GABA) A receptor, beta 2 | 2.71 | 0.00731008 | NB |
| HEPACAM | hepatic and glial cell adhesion molecule | 2.71 | 0.02247963 | NB |
| RP11-903H12.2 | | 2.71 | 0.027650005 | NB |
| CTA-211A9.5 | | 2.71 | 0.006592317 | NB |
| BHLHB9 | basic helix-loop-helix domain containing, class B, 9 | 2.71 | 0.005329798 | NB |
| RP11-157D18.2 | | 2.7 | 0.020318802 | NB |
| RP11-317B7.2 | | 2.7 | 0.038991906 | NB |
| FAM198B | family with sequence similarity 198, member B | 2.7 | 0.015742933 | NB |
| FAM81B | family with sequence similarity 81, member B | 2.7 | 0.042306582 | NB |
| RCAN2 | regulator of calcineurin 2 | 2.7 | 0.022360081 | NB |
| RP11-533F5.2 | | 2.69 | 0.025144552 | NB |
| RP11-597D13.9 | | 2.69 | 0.022360081 | NB |
| KLHL31 | kelch-like family member 31 | 2.69 | 0.031835936 | NB |
| EIF3EP1 | eukaryotic translation initiation factor 3, subunit E pse [symbol] | 2.69 | 0.000438786 | NB |
| MLLT11 | myeloid/lymphoid or mixed-lineage leukemia (trithora [symbol] | 2.68 | 0.003430441 | NB |
| SERPINC1 | serpin peptidase inhibitor, clade C (antithrombin), me [symbol] | 2.68 | 0.004785714 | NB |
| RP11-280F2.1 | | 2.68 | 0.00713094 | NB |
| PCDHGA3 | protocadherin gamma subfamily A, 3 | 2.68 | 0.003839527 | NB |
| SPRY4 | sprouty homolog 4 (Drosophila) | 2.68 | 0.039288394 | NB |
| BMP6 | bone morphogenetic protein 6 | 2.68 | 0.008070194 | NB |
| SSU72P8 | SSU72 pseudogene 8 | 2.68 | 0.006562428 | NB |
| PEX5L-AS1 | PEX5L antisense RNA 1 | 2.66 | 0.033545013 | NB |
| LRP4 | low density lipoprotein receptor-related protein 4 | 2.66 | 0.011925792 | NB |
| NPM1P8 | nucleophosmin 1 (nucleolar phosphoprotein B23, num [symbol] | 2.66 | 0.027941793 | NB |
| SHROOM1 | shroom family member 1 | 2.65 | 0.008070194 | NB |
| HMGN2P10 | high mobility group nucleasomal binding domain 2 pse [symbol] | 2.65 | 0.045518278 | NB |
| DGAT2L6 | diacylglycerol O-acyltransferase 2-like 6 | 2.65 | 0.018415671 | NB |
| RP11-944L7.4 | | 2.64 | 0.040821352 | NB |
| OTOP1 | otopetrin 1 | 2.64 | 0.03495366 | NB |
| PCDHGC3 | protocadherin gamma subfamily C, 3 | 2.64 | 0.02643572 | NB |
| GSG1 | germ cell associated 1 | 2.64 | 0.001853615 | NB |
| VWA3A | von Willebrand factor A domain containing 3A | 2.64 | 0.0186415 | NB |
| LMOD3 | leiomodin 3 (fetal) | 2.63 | 0.01785722 | NB |
| PIRT | phosphoinositide-interacting regulator of transient rec [symbol] | 2.63 | 0.00447648 | NB |
| RPL36P4 | ribosomal protein L36 pseudogene 4 | 2.63 | 0.007320475 | NB |
| THBS4 | thrombospondin 4 | 2.62 | 0.049100171 | NB |
| RP11-482D24.3 | | 2.62 | 0.013181478 | NB |
| SULT1A1 | sulfotransferase family, cytosolic, 1A, phenol-preferrin [symbol] | 2.62 | 0.018812935 | NB |
| CEACAM8 | carcinoembryonic antigen-related cell adhesion molec [symbol] | 2.62 | 0.02287369 | NB |
| LNX1 | ligand of numb-protein X 1, E3 ubiquitin protein ligase | 2.61 | 0.014371388 | NB |
| GRM7 | glutamate receptor, metabotropic 7 | 2.6 | 0.020255042 | NB |
| RP11-651P23.2 | | 2.6 | 0.029382731 | NB |
| AC097467.2 | | 2.6 | 0.033794271 | NB |
| DBX2 | developing brain homeobox 2 | 2.6 | 0.003036454 | NB |
| AL445989.1 | | 2.6 | 0.03995294 | NB |
| TEKT5 | tektin 5 | 2.6 | 0.016408594 | NB |
| CAGE1 | cancer antigen 1 | 2.59 | 0.012569126 | NB |
| OR52E6 | olfactory receptor, family 52, subfamily E, member 6 | 2.59 | 0.016753819 | NB |
| CTD-2314B22.3 | | 2.59 | 0.004785714 | NB |
| ZNF177 | zinc finger protein 177 | 2.59 | 0.045638568 | NB |
| AP000281.1 | | 2.59 | 0.043523654 | NB |
| CELA2B | chymotrypsin-like elastase family, member 2B | 2.58 | 0.001487356 | NB |
| LHCGR | luteinizing hormone/choriogonadotropin receptor | 2.58 | 0.039288394 | NB |
| GRID2 | glutamate receptor, ionotropic, delta 2 | 2.58 | 0.019899858 | NB |
| LRRC17 | leucine rich repeat containing 17 | 2.58 | 0.001483816 | NB |
| OR51I2 | olfactory receptor, family 51, subfamily I, member 2 | 2.58 | 0.007916312 | NB |
| RP11-166D19.1 | | 2.58 | 0.023509294 | NB |
| ZDHHC22 | zinc finger, DHHC-type containing 22 | 2.58 | 0.009476909 | NB |
| KSR1 | kinase suppressor of ras 1 | 2.58 | 0.014371388 | NB |
| EMID1 | EMI domain containing 1 | 2.58 | 0.018812935 | NB |
| OR6K3 | olfactory receptor, family 6, subfamily K, member 3 | 2.57 | 0.039717587 | NB |
| SLC38A3 | solute carrier family 38, member 3 | 2.57 | 0.04352477 | NB |
| B3GALNT1 | beta-1,3-N-acetylgalactosaminyltransferase 1 (globosid [symbol] | 2.57 | 0.033656613 | NB |
| CTD-2337A12.1 | | 2.57 | 0.019575576 | NB |
| GALNT8 | polypeptide N-acetylgalactosa minyltransferase 8 | 2.57 | 0.033656613 | NB |
| SAGE1 | sarcoma antigen 1 | 2.57 | 0.030546079 | NB |
| OR52D1 | olfactory receptor, family 52, subfamily D, member 1 | 2.56 | 0.029028339 | NB |
| ALX4 | ALX homeobox 4 | 2.56 | 0.009204866 | NB |
| KCNMB4 | potassium large conductance calcium-activated channe [symbol] | 2.56 | 0.036386177 | NB |
| AL589743.1 | | 2.56 | 0.001007228 | NB |
| AP000322.53 | | 2.56 | 0.016371906 | NB |
| HBXIPP1 | | 2.55 | 0.024542839 | NB |
| CHN1 | chimerin 1 | 2.55 | 0.028688235 | NB |
| TACR3 | tachykinin receptor 3 | 2.55 | 0.011399948 | NB |
| AC008265.2 | | 2.55 | 0.033214335 | NB |
| STRADBP1 | STE20-related kinase adaptor beta pseudogene 1 | 2.55 | 0.011576251 | NB |
| C9orf57 | | 2.55 | 0.019540488 | NB |
| DPF3 | D4, zinc and double PHD fingers, famiy 3 | 2.55 | 0.039288394 | NB |
| GOLGA6B | golgin A6 family, member B | 2.55 | 0.039316473 | NB |
| GNAS-AS1 | GNAS antisense RNA 1 | 2.55 | 0.000379081 | NB |
| RP13-140E4.1 | | 2.55 | 0.021446576 | NB |
| LL0XNC01-1161E7.1 | | 2.55 | 0.02857165 | NB |
| OR51I1 | olfactory receptor, family 51, subfamily I, member 1 | 2.54 | 0.015079112 | NB |
| UNC13C | unc-13 homolog C (C. elegans) | 2.54 | 0.04694431 | NB |
| RP11-1079K10.1 | | 2.54 | 0.038228592 | NB |
| GOLGA6L16P | golgin A6 family-like 16, pseudogene | 2.54 | 0.017521647 | NB |
| MMEL1 | membrane metallo-endopeptidase-like 1 | 2.53 | 0.027650005 | NB |
| CTD-2330J20.2 | | 2.53 | 0.020779381 | NB |
| RRM2P3 | ribonucleotide reductase M2 polypeptide pseudogene | 2.53 | 0.021040385 | NB |
| AGTR1 | angiotensin II receptor, type 1 | 2.52 | 0.039316473 | NB |
| RELN | reelin | 2.52 | 0.02052367 | NB |
| HMGN1P18 | high mobility group nudeasome binding domain 1 pse [symbol] | 2.52 | 0.035185369 | NB |
| ANGPT1 | angiopoietin 1 | 2.52 | 0.024328524 | NB |
| GRIK1 | glutamate receptor, ionotropic, kainate 1 | 2.52 | 0.000881664 | NB |
| RP11-267N12.3 | | 2.51 | 0.003839527 | NB |
| KIAA1239 | NACHT and WD repeat domain containing 2 | 2.51 | 0.00099915 | NB |
| AGBL3 | ATP/GTP binding protein-like 3 | 2.51 | 0.024328524 | NB |
| RP11-32B5.2 | | 2.51 | 0.028184742 | NB |
| ATP2B3 | ATPase, Ca++ transporting, plasma membrane 3 | 2.51 | 0.033698056 | NB |
| TAS1R1 | taste receptor, type 1, member 1 | 2.5 | 0.017138695 | NB |
| ANKRD20A14P | ankyrin repeat domain 20 family, member A14, pseudo [symbol] | 2.5 | 0.049100171 | NB |
| RGS5 | regulator of G-protein signaling 5 | 2.5 | 0.002421425 | NB |
| SOWAHA | sosondowah ankyrin repeat domain family member A | 2.5 | 0.030546079 | NB |
| OR52E4 | olfactory receptor, family 52, subfamily E, member 4 | 2.5 | 0.023557449 | NB |
| ANO3 | anoctamin 3 | 2.5 | 0.02019187 | NB |
| TMEFF1 | transmembrane protein with EGF-like and two follistati [symbol] | 2.49 | 0.005477973 | NB |
| C12orf28 | | 2.49 | 0.035978444 | NB |
| CORIN | corin, serine peptidase | 2.48 | 0.033656613 | NB |
| RP11-309L24.6 | | 2.48 | 0.014969485 | NB |
| SYNM | synemin, intermediate filament protein | 2.48 | 0.003059735 | NB |
| PDHA2 | pyruvate dehydrogenase (lipoamide) alpha 2 | 2.47 | 0.015181716 | NB |
| ANGPT2 | angiopcietin 2 | 2.47 | 0.004785714 | NB |
| S1PR3 | sphingosine-1-phosphate receptor 3 | 2.47 | 0.031092971 | NB |
| ANKRD20A8P | ankyrin repeat domain 20 family, member A8, pseudog [symbol] | 2.46 | 0.031092971 | NB |
| OBP2B | odorant binding protein 2B | 2.46 | 0.046783458 | NB |
| HHEX | hematopoietically expressed homeobox | 2.46 | 0.008070194 | NB |
| AC007731.1 | | 2.46 | 0.045238769 | NB |
| CLCN4 | chloride channel, voltage-sensitive 4 | 2.46 | 0.018812935 | NB |
| HSPA7 | heat shock 70 kDa protein 7(HSP70B) | 2.45 | 0.031092971 | NB |
| C7orf58 | | 2.45 | 0.022360081 | NB |
| ANKRD20A3 | ankyrin repeat domain 20 family, member A3 | 2.45 | 0.028688235 | NB |
| C2orf66 | | 2.44 | 0.024790121 | NB |
| OR56B4 | olfactory receptor, family 56, subfamily B, member 4 | 2.44 | 0.020968365 | NB |
| PCDHGB1 | protocadherin gamma subfamily B, 1 | 2.43 | 0.022360081 | NB |
| RP1-97D16.1 | | 2.43 | 0.02857165 | NB |
| PPP1R9A | protein phosphatase 1, regulatory subunit 9A | 2.43 | 0.036386177 | NB |
| TMC5 | transmembrane channel-like 5 | 2.43 | 0.033351877 | NB |
| TEX11 | testis expressed 11 | 2.43 | 0.01305666 | NB |
| PADI6 | peptidyl arginine deiminase, type VI | 2.42 | 0.048733769 | NB |
| RP11-481K9.4 | | 2.42 | 0.0186415 | NB |
| RP11-496I2.6 | | 2.42 | 0.009839457 | NB |
| RP11-310A13.2 | | 2.41 | 0.01373806 | NB |
| RP1-66E7.1 | | 2.41 | 0.045812142 | NB |
| RP11-463J10.2 | | 2.41 | 0.031092971 | NB |
| SULT1C4 | sulfotransferase family, cytosolic, 1C, member 4 | 2.4 | 0.003430441 | NB |
| AC073629.2 | | 2.4 | 0.025822313 | NB |
| CTD-2349P21.1 | | 2.4 | 0.002682049 | NB |
| TCN1 | transcobalamin I (vitamin B12 binding protein, R binde [symbol] | 2.39 | 0.039288394 | NB |
| SYT16 | synaptotagmin XVI | 2.39 | 0.00346671 | NB |
| RP11-454K24.1 | | 2.39 | 0.007806012 | NB |
| SLC35G2 | solute carrier farniy 35, member G2 | 2.38 | 0.028688235 | NB |
| RP11-321E8.1-001 | | 2.38 | 0.002608342 | NB |
| RP11-6O2.4 | | 2.38 | 0.003839527 | NB |
| HSPA6 | heat shock 70 kDa protein 6 (HSP70B′) | 2.37 | 0.024328524 | NB |
| RP11-136C24.1 | | 2.37 | 0.042079896 | NB |
| AC073343.1 | | 2.37 | 0.004185714 | NB |
| GAGE10 | G antigen 10 | 2.37 | 0.047897625 | NB |
| ANKRD20A2 | ankyrin repeat domain 20 family, member A2 | 2.36 | 0.022360081 | NB |
| DUSP13 | dual specificity phosphatase 13 | 2.36 | 0.016199164 | NB |
| ITM2C | integral membrane protein 2C | 2.35 | 0.001902564 | NB |
| RP11-397E7.2 | | 2.35 | 0.039148946 | NB |
| OSMR | oncostatin M receptor | 2.35 | 0.042370292 | NB |
| RP11-10F11.2 | | 2.35 | 0.013831074 | NB |
| TFPI2 | tissue factor pathway inhibitor 2 | 2.35 | 0.047168612 | NB |
| RP11-530N7.2 | | 2.35 | 0.033852011 | NB |
| POU5F1B | POU class 5 homeobox 1B | 2.34 | 0.045478051 | NB |
| TECTA | tectorin alpha | 2.34 | 0.013100955 | NB |
| RASL10B | RAS-like, family 10, member B | 2.34 | 0.09883796 | NB |
| CYP2G1P | cytochrome P450, family 2, subfamily G, polypeptide 1 | 2.34 | 0.012569126 | NB |
| RP4-610C12.4 | | 2.34 | 0.009966462 | NB |
| OR11L1 | olfactory receptor, family 11, subfamily L, member 1 | 2.33 | 0.029382731 | NB |
| CNTN6 | contactin 6 | 2.33 | 0.031092971 | NB |
| C4orf19 | | 2.33 | 0.004949546 | NB |
| YIPF7 | Yip1 domain family, member 7 | 2.33 | 0.003640543 | NB |
| PCDHGC5 | protocadherin gamma subfamily C, 5 | 2.33 | 0.005329798 | NB |
| ADGB | androglobin | 2.33 | 0.022638595 | NB |
| PSMC1P3 | proteasome (prosome, macropain) 26S subunit, ATPas [symbol] | 2.33 | 0.043902532 | NB |
| FGF2 | fibroblast growth factor 2 (basic) | 2.32 | 0.022360081 | NB |
| AC008537.2 | | 2.32 | 0.002180177 | NB |
| CTD-2224J9.4 | | 2.32 | 0.032296489 | NB |
| CSPG5 | chondroitin sulfate proteoglycan 5 (neuroglycan C) | 2.31 | 0.028688235 | NB |
| RP11-254A17.1 | | 2.31 | 0.01373806 | NB |
| NACAD | NAC alpha domain containing | 2.31 | 0.031092971 | NB |
| LMOD2 | leiomodin 2 (cardiac) | 2.31 | 0.047168612 | NB |
| BAALC | brain and acute leukemia, cytoplasmic | 2.31 | 0.045638568 | NB |
| RP11-84C10.2 | | 2.31 | 0.042339272 | NB |
| HNRNPA1P24 | heterogeneous nuclear ribonucleoprotein A1 pseudoge [symbol] | 2.3 | 0.017952081 | NB |
| KCNMB2 | potassium large conductance calcium-activated channe [symbol] | 2.3 | 0.02287369 | NB |
| NREP | neuronal regeneration related protein | 2.3 | 0.003430441 | NB |
| PCMTD1P3 | protein-L-isoaspartate (D-aspartate) O-methyltransfera [symbol] | 2.3 | 0.015181716 | NB |
| ADRB3 | adrenoceptor beta 3 | 2.3 | 0.035978444 | NB |
| PA2G4P2 | proliferation-associated 2G4 pseudogene 2 | 2.3 | 0.03877208 | NB |
| HOXD10 | homeobox D10 | 2.29 | 0.002421425 | NB |
| GNG5P3 | guanine nucleotide binding protein (G protein), gamma [symbol] | 2.29 | 0.011925792 | NB |
| KCNJ8 | potassium inwardly-rectifying channel, subfamily J, me [symbol] | 2.29 | 0.02643572 | NB |
| RP11-182J1.13 | | 2.29 | 0.049100171 | NB |
| LIMS3 | LIM and senescent cell antigen-like domains 3 | 2.28 | 0.032616447 | NB |
| HOXA-AS2 | HOXA cluster antisense RNA 2 | 2.28 | 0.028688235 | NB |
| TRPC4 | transient receptor potential cation channel, subfamily [symbol] | 2.28 | 0.02052367 | NB |
| DNER | delta/notch-like EGF repeat containing | 2.27 | 0.013528999 | NB |
| C5orf60 | | 2.27 | 0.030628105 | NB |
| NECAB1 | N-terminal EF-hand calcium binding protein 1 | 2.27 | 0.022828147 | NB |
| RP11-144A16.1 | | 2.27 | 0.014371388 | NB |
| LINC00552 | long intergenic non-protein coding RNA 552 | 2.27 | 0.04104249 | NB |
| MEG8 | maternally expressed 8 (non-protein coding) | 2.27 | 0.022042996 | NB |
| AC005562.3 | | 2.27 | 0.01377845 | NB |
| NAP1L5 | nucleosome assembly protein 1-like 5 | 2.26 | 0.008070194 | NB |
| NXPH1 | neurexophilin 1 | 2.26 | 0.046077602 | NB |
| BBOX1 | butyrobetaine (gamma), 2-oxoglutarate dioxygenase (g [symbol] | 2.26 | 0.045478051 | NB |
| SCN4B | sodium channel, voltage-gated, type IV, beta subunit | 2.26 | 0.024328524 | NB |
| ZFP82 | ZFP82 zinc finger protein | 2.26 | 0.045638568 | NB |
| RP1-83M4.2 | | 2.25 | 0.004965109 | NB |
| FABP9 | fatty acid binding protein 9, testis | 2.25 | 0.004124703 | NB |
| NEB | nebulin | 2.24 | 0.049100171 | NB |
| SPP1 | secreted phosphoprotein 1 | 2.24 | 0.049100171 | NB |
| NPY6R | neuropeptide Y receptor Y6 (pseudogene) | 2.24 | 0.049100171 | NB |
| PCDHB12 | protocadherin beta 12 | 2.24 | 0.002421425 | NB |
| RP1-95L4.4 | | 2.24 | 0.017221389 | NB |
| C15orf26 | | 2.24 | 0.02052367 | NB |
| IGSF1 | immunoglobuin superfamily, member 1 | 2.24 | 0.037056891 | NB |
| COL6A4P1 | collagen, type VI, alpha 4 pseudogene 1 | 2.23 | 0.030546079 | NB |
| PCDHB10 | protocadherin beta 10 | 2.23 | 0.005329798 | NB |
| DPYSL3 | dihydropyrimidinase-like 3 | 2.23 | 0.031092971 | NB |
| HOXA5 | homeobox A5 | 2.23 | 0.005329798 | NB |
| RP11-561C5.5 | | 2.23 | 0.009531305 | NB |
| AP000525.10 | | 2.23 | 0.036386177 | NB |
| FOXO6 | forkhead box O6 | 2.22 | 0.036386177 | NB |
| MIR571 | microRNA 571 | 2.22 | 0.041739925 | NB |
| SLC22A7 | solute carrier family 22 (organic anion transporter), me [symbol] | 2.22 | 0.038448428 | NB |
| AL589743.2 | | 2.22 | 0.035185369 | NB |
| DLK1 | delta-like 1 homolog (Drosophila) | 2.22 | 0.047935027 | NB |
| RP11-316M1.11 | | 2.21 | 0.02643572 | NB |
| C1orf189 | | 2.21 | 0.027138346 | NB |
| RP11-182J1.14 | | 2.21 | 0.013100955 | NB |
| snoU2_19 | | 2.21 | 0.031216315 | NB |
| GSTM3 | glutathione S-transferase mu 3 (brain) | 2.2 | 0.024328524 | NB |
| TMEM30C | transmembrane protein 30C | 2.2 | 0.019201979 | NB |
| CSGALNACT1 | chondroitin sulfate N-acetylgalactosaminyltransferase [symbol] | 2.2 | 0.004785714 | NB |
| NAP1L1P1 | nucleosome assembly protein 1-like 1 pseudogene 1 | 2.2 | 0.032737245 | NB |
| RP11-522B15.6 | | 2.2 | 0.04876748 | NB |
| PRAMEF15 | PRAME famity member 15 | 2.19 | 0.046783458 | NB |
| HPCA | hippocalcin | 2.19 | 0.009764943 | NB |
| ISPD | isoprenoid synthase domain containing | 2.19 | 0.001483816 | NB |
| RP11-365P13.3 | | 2.19 | 0.021985463 | NB |
| AL132709.8 | | 2.19 | 0.045588897 | NB |
| GOLGA6A | golgin A6 family, member A | 2.19 | 0.045638568 | NB |
| AC048382.4 | | 2.19 | 0.02052367 | NB |
| MYO15A | myosin XVA | 2.19 | 0.031092971 | NB |
| C1orf61 | | 2.18 | 0.042306582 | NB |
| KCNJ10 | potassium inwardly-rectifying channel, subfamily J, me [symbol] | 2.18 | 0.045638568 | NB |
| TNNI1 | troponin I type 1 (skeletal, slow) | 2.18 | 0.049758592 | NB |
| SCN2A | sodium channel, voltage-gated, type II, alpha subunit | 2.18 | 0.012651023 | NB |
| NSUN7 | NOP2/Sun domain family, member 7 | 2.18 | 0.049100171 | NB |
| PRSS48 | protease, serine, 48 | 2.18 | 0.018812935 | NB |
| HBEGF | heparin-binding EGF-like growth factor | 2.18 | 0.001902564 | NB |
| FAM75A6 | SPATA31 subfamily A, member 6 | 2.18 | 0.026579731 | NB |
| IMPDH1P11 | IMP (inosine monophosphate) dehydrogenase 1 pseud [symbol] | 2.18 | 0.037640265 | NB |
| RP11-145A3.4 | | 2.17 | 0.011547361 | NB |
| 3-Mar | membrane-associated ring finger (C3HC4) 3, E3 ubiquit [symbol] | 2.17 | 0.002724323 | NB |
| CAMK2B | calcium/calmodulin-dependent protein kinase II beta | 2.17 | 0.03791799 | NB |
| C9orf171 | | 2.17 | 0.005662083 | NB |
| RBM20 | RNA binding motif protein 20 | 2.17 | 0.028688235 | NB |
| ASCL3 | achaete-scute family bHLH transcription factor 3 | 2.17 | 0.021083625 | NB |
| PDE4B | phosphodiesterase 4B, cAMP-specific | 2.16 | 0.033656613 | NB |
| SERPINE2 | serpin peptidase inhibitor, clade E (nexin, plasminogen [symbol] | 2.16 | 0.008917729 | NB |
| PCDHGB2 | protocadherin gamma subfamily B, 2 | 2.16 | 0.000280919 | NB |
| ZNF204P | zinc finger protein 204, pseudogene | 2.16 | 0.045512017 | NB |
| 14-Sep | septin 14 | 2.16 | 0.013119454 | NB |
| RP11-661G16.2 | | 2.16 | 0.02052367 | NB |
| SRP68P3 | signal recognition particle 68 kDa pseudogene 3 | 2.16 | 0.029028339 | NB |
| NEFH | neurofilament, heavy polypeptide | 2.16 | 0.042370292 | NB |
| ADCY10 | adenylate cyclase 10 (soluble) | 2.15 | 0.002148321 | NB |
| RP11-472B18.1 | | 2.15 | 0.004785714 | NB |
| RP11-673E1.3 | | 2.15 | 0.004290131 | NB |
| AE000662.92 | | 2.14 | 0.039148946 | NB |
| KCNMB3P1 | potassium large conductance calcium-activated channe [symbol] | 2.14 | 0.008070194 | NB |
| MIR1231 | microRNA 1231 | 2.13 | 0.033553777 | NB |
| WDR64 | WD repeat domain 64 | 2.13 | 0.012073496 | NB |
| AC004980.10 | | 2.13 | 0.000671309 | NB |
| SLC26A3 | solute carrier family 26 (anion exchanger), member 3 | 2.13 | 0.039253648 | NB |
| SNORA49 | small nucleolar RNA, H/ACA box 49 | 2.13 | 0.022360081 | NB |
| ESRRB | estrogen-related receptor beta | 2.13 | 0.042306582 | NB |
| AC011551.2 | | 2.13 | 0.026864606 | NB |
| ARHGAP29 | Rho GTPase activating protein 29 | 2.12 | 0.039288394 | NB |
| AL583842.6 | | 2.12 | 0.036386177 | NB |
| LRRC9 | leucine rich repeat containing 9 | 2.12 | 0.02643572 | NB |
| NID1 | nidogen 1 | 2.11 | 0.004785714 | NB |
| RP11-13J8.1 | | 2.11 | 0.004220588 | NB |
| PCDHB18 | | 2.11 | 0.011925792 | NB |
| ZNF679 | zinc finger protein 679 | 2.11 | 0.046783458 | NB |
| RP11-959I15.1 | | 2.11 | 0.02939697 | NB |
| AP000593.6 | | 2.11 | 0.026579731 | NB |
| TSPY26P | testis specific protein, Y-linked 26, pseudogene | 2.11 | 0.042370292 | NB |
| FBLIM1 | filamin binding LIM protein 1 | 2.1 | 0.045638568 | NB |
| PAPPA2 | pappalysin 2 | 2.1 | 0.045478051 | NB |
| OFD1P17 | OFD1 pseudogene 17 | 2.1 | 0.022360081 | NB |
| RP11-43D2.6 | | 2.1 | 0.049100171 | NB |
| GRAMD3 | GRAM domain containing 3 | 2.1 | 0.036386177 | NB |
| ICK | intestinal cell (MAK-like) kinase | 2.1 | 0.015742933 | NB |
| EPB41L2 | erythrocyte membrane protein band 4.1-like 2 | 2.1 | 0.001902564 | NB |
| TUSC3 | tumor suppressor candidate 3 | 2.1 | 0.02052367 | NB |
| RP11-1102P16.1 | | 2.1 | 0.033545013 | NB |
| RPL19P14 | ribosomal protein L19 pseudogene 14 | 2.1 | 0.005477973 | NB |
| RP11-654A16.3 | | 2.1 | 0.003321047 | NB |
| TMPRSS9 | transmembrane protease, serine 9 | 2.1 | 0.018812935 | NB |
| FNBP1L | formin binding protein 1-like | 2.09 | 0.005329798 | NB |
| ASNSP5 | asparagine synthetase pseudogene 5 | 2.09 | 0.024790121 | NB |
| AC007272.3 | | 2.09 | 0.004220588 | NB |
| PLK2 | polo-like kinase 2 | 2.09 | 0.036386177 | NB |
| PLAC8L1 | PLAC8-like 1 | 2.09 | 0.021985463 | NB |
| GAPDHP73 | glyceraldehyde-3-phosphate dehydrogenase pseudoge [symbol] | 2.09 | 0.039934696 | NB |
| RP11-529J17.2 | | 2.09 | 0.005329798 | NB |
| MAP7D2 | MAP 7 domain containing 2 | 2.09 | 0.047168612 | NB |
| ASPRV1 | aspartic peptidase, retroviral-like 1 | 2.08 | 0.017221389 | NB |
| SLC10A4 | solute carrier family 10, member 4 | 2.08 | 0.015740659 | NB |
| DCHS2 | dachsous cadherin-related 2 | 2.08 | 0.036386177 | NB |
| PCDHB15 | protocadherin beta 15 | 2.08 | 0.001681771 | NB |
| RP11-94K8.1 | | 2.08 | 0.045295878 | NB |
| TMC2 | transmembrane channel-like 2 | 2.08 | 0.017138695 | NB |
| CTD-2514C3.1 | | 2.08 | 0.003430441 | NB |
| C3orf30 | | 2.07 | 0.034606296 | NB |
| KCTD16 | potassium channel tetramerization domain containing | 2.07 | 0.035336374 | NB |
| BACH1-IT1 | BACH1 intronic transcript 1 (non-protein coding) | 2.07 | 0.000671309 | NB |
| RP11-1114A5.4 | | 2.07 | 0.031092971 | NB |
| LEPR | leptin receptor | 2.06 | 0.013100955 | NB |
| PCDHB19P | protocadherin beta 19 pseudogene | 2.06 | 0.008917729 | NB |
| SPINK7 | serine peptidase inhibitor, Kazal type 7 (putative) | 2.06 | 0.021373132 | NB |
| LCTL | lactase-like | 2.06 | 0.049100171 | NB |
| AC011718.2 | | 2.06 | 0.016199164 | NB |
| ZCCHC12 | zinc finger, CCHC domain containing 12 | 2.06 | 0.004421152 | NB |
| RP11-1007J8.1 | | 2.05 | 0.030204228 | NB |
| S100G | S100 calcium binding protein G | 2.05 | 0.011590706 | NB |
| C3orf74 | | 2.04 | 0.018624954 | NB |
| NAALADL2 | N-acetylated alpha-linked acidic dipeptidase-like 2 | 2.04 | 0.042370292 | NB |
| SPRY1 | sprouty homolog 1, antagonist of FGF signaling (Droso [symbol] | 2.04 | 0.003839527 | NB |
| MIR550A2 | microRNA 550a-2 | 2.04 | 0.020508813 | NB |
| FAM55A | neurexophilin and PC-esterase domain family, member [symbol] | 2.04 | 0.04876748 | NB |
| SP6 | Sp6 transcription factor | 2.04 | 0.016408594 | NB |
| RP5-1068H6.3 | | 2.04 | 0.017824327 | NB |
| HMGN2P23 | high mobility group nucleosomal binding domain 2 pse [symbol] | 2.03 | 0.023511088 | NB |
| PLSCR4 | phospholipid scramblase 4 | 2.03 | 0.008070194 | NB |
| CTD-2292P10.4 | uncharacterized LOC100288181 | 2.03 | 0.04883796 | NB |
| AQP7P2 | aquaporin 7 pseudogene 2 | 2.03 | 0.047168612 | NB |
| IFLTD1 | | 2.03 | 0.038978796 | NB |
| RP11-423O2.1 | | 2.02 | 0.009084306 | NB |
| CCDC136 | coiled-coil domain containing 136 | 2.02 | 0.005329798 | NB |
| ATP2B1 | ATPase, Ca++ transporting, plasma membrane 1 | 2.02 | 0.003059735 | NB |
| ASIP | agouti signaling protein | 2.02 | 0.031092971 | NB |
| DPPA3P1 | developmental pluripotency associated 3 pseudogene [symbol] | 2.02 | 0.031391184 | NB |
| AC007563.1 | | 2.01 | 0.027988697 | NB |
| SEMA5A | sema domain, seven thrombospondin repeats (type 1 a [symbol] | 2.01 | 0.033656613 | NB |
| SCARNA15 | small Cajal body-specific RNA 15 | 2.01 | 0.033351877 | NB |
| TRHR | thyrotropin-releasing hormone receptor | 2.01 | 0.047168612 | NB |
| RPL7L1P12 | ribosomal protein 17-like 1 pseudogene 12 | 2.01 | 0.024790121 | NB |
| RP11-138H8.5 | | 2.01 | 0.039288394 | NB |
| DUSP15 | dual specificity phosphatase 15 | 2.01 | 0.045638568 | NB |
| ZNF75D | zinc finger protein 75D | 2.01 | 0.02643572 | NB |
| MIR211 | microRNA 211 | 654.53 | 0.003258841 | CB |
| TRGJP2 | T cell receptor gamma joining P2 | 98.51 | 0.018744092 | CB |
| IGHJ2 | immunoglobulin heavy joining 2 | 92.82 | 0.048733769 | CB |
| RP11-259G18.2 | | 69.31 | 0.039288394 | CB |
| TRBV7-7 | T cell receptor beta variable 7-7 | 57.74 | 0.00541274 | CB |
| IGKV6-21 | immunoglobulin kappa variable 6-21 (non-functional) | 49.85 | 0.037395217 | CB |
| IGHJ1 | immunoglobulin heavy joining 1 | 44.48 | 0.04073483 | CB |
| AC105247.1 | | 31.61 | 0.014702641 | CB |
| MIR513A2 | microRNA 513a-2 | 30.81 | 0.024507002 | CB |
| FAM69C | family with sequence similarity 69, member C | 28.52 | 0.028374775 | CB |
| ZBTB40-IT1 | ZBTB40 intronic transcript 1 (non-protein coding) | 28.24 | 0.029540253 | CB |
| TRBV5-7 | T cell receptor beta variable 5-7 (non-functional) | 27.78 | 0.011689051 | CB |
| TRPM1 | transient receptor potential cation channel, subfamily [symbol] | 25.45 | 0.003430441 | CB |
| TRBV6-9 | T cell receptor beta variable 6-9 | 25.33 | 0.036277389 | CB |
| IGHV3-11 | immunoglobulin heavy variable 3-11 (gene/pseudogen [symbol] | 24.99 | 0.006019354 | CB |
| RP1-128O3.5 | | 24.91 | 0.000845487 | CB |
| RP11-554D14.3 | | 24.51 | 0.043395953 | CB |
| MIR185 | microRNA 185 | 24.08 | 0.046543212 | CB |
| RP11-544M22.3 | | 23.4 | 0.006210424 | CB |
| RP11-120D12.2 | | 23.16 | 0.00346671 | CB |
| RP11-481A20.4 | | 22.91 | 0.023557449 | CB |
| IGKV3-11 | immunoglobulin kappa variable 3-11 | 22.63 | 0.019304276 | CB |
| IGHV1-2 | immunoglobulin heavy variable 1-2 | 22.09 | 0.012651023 | CB |
| TRBV11-1 | T cell receptor beta variable 11-1 | 21.8 | 0.035979007 | CB |
| RP11-118B22.1 | | 21.53 | 0.014367798 | CB |
| IGKV1-6 | immunoglobulin kappa variable 1-6 | 19.5 | 0.017656956 | CB |
| IGKV3D-11 | immunoglobulin kappa variable 3D-11 | 19.47 | 0.019058997 | CB |
| RP11-170L3.7 | | 18.78 | 0.028374775 | CB |
| IGHV3-23 | immunoglobulin heavy variable 3-23 | 17.5 | 0.02287369 | CB |
| IGKV1-12 | immunoglobulin kappa variable 1-12 | 16.37 | 0.033794271 | CB |
| IL21 | interleukin 21 | 16.05 | 0.024189646 | CB |
| IGHV1-24 | immunoglobulin heavy variable 1-24 | 15.87 | 0.001953399 | CB |
| IGHV2-70 | immunoglobulin heavy variable 2-70 | 15.8 | 0.001854517 | CB |
| RP11-118F2.3 | | 15.48 | 0.010626103 | CB |
| RPS3AP33 | ribosomal protein S3a pseudogene 33 | 15.32 | 0.012125647 | CB |
| TRBV7-3 | T cell receptor beta variable 7-3 | 15.2 | 0.013029879 | CB |
| CXorf1 | | 14.71 | 0.009648355 | CB |
| ADCY2 | adenylate cyclase 2 (brain) | 14.68 | 0.013827431 | CB |
| GPR25 | G protein-coupled receptor 25 | 14.14 | 0.035978626 | CB |
| RP4-673D20.5 | | 14.13 | 0.008612991 | CB |
| RPL36AP40 | ribosomal protein L36a pseudogene 40 | 13.94 | 0.047090742 | CB |
| RP11-215P8.1 | | 13.68 | 0.020435079 | CB |
| RP11-348B17.1 | | 13.68 | 0.008070194 | CB |
| RP4-738P11.4 | | 13.46 | 0.03745438 | CB |
| IGKV1D-12 | immunoglobulin kappa variable 1D-12 | 13.21 | 0.04213223 | CB |
| RP11-386I23.1 | | 13.2 | 0.002970807 | CB |
| IGKV4-1 | immunoglobulin kappa variable 4-1 | 12.83 | 0.021040385 | CB |
| IGLV1-40 | immunoglobulin lambda variable 1-40 | 12.37 | 0.045638568 | CB |
| GTSF1L | gametocyte specific factor 1-like | 12 | 0.017173685 | CB |
| IGKV1D-16 | immunoglobulin kappa variable 1D-16 | 11.57 | 0.031078338 | CB |
| IGHV4-28 | immunoglobulin heavy variable 4-28 | 11.31 | 0.048226887 | CB |
| GPR79 | G protein-coupled receptor 79, pseudogene | 11.3 | 0.033590863 | CB |
| IGLC6 | immunoglobulin lambda constant 6 [Kern + Oz− marker, [symbol] | 11.06 | 0.024790121 | CB |
| IGKV2-28 | immunoglobulin kappa variable 2-28 | 10.81 | 0.01377845 | CB |
| CTD-2201E18.1 | | 10.79 | 0.018497346 | CB |
| IGHV3-64 | immunoglobulin heavy variable 3-64 | 10.53 | 0.047995343 | CB |
| AC009892.10 | | 10.51 | 0.015181716 | CB |
| IGHG1 | immunoglobulin heavy constant gamma 1 (G1m marke [symbol] | 10.4 | 0.036386177 | CB |
| IGKV1-9 | immunoglobulin kappa variable 1-9 | 10.34 | 0.028975593 | CB |
| RP11-500G10.4 | | 10.13 | 0.021494609 | CB |
| IGKV2D-28 | immunoglobulin kappa variable 2D-28 | 10.05 | 0.019434049 | CB |
| RP11-281P11.1 | | 9.68 | 0.006912894 | CB |
| IGHV3-49 | immunoglobulin heavy variable 3-49 | 9.68 | 0.022638595 | CB |
| CKMT1A | creatine kinase, mitochondrial 1A | 9.65 | 0.036386177 | CB |
| RP11-166B2.5 | | 9.58 | 0.012166712 | CB |
| RP11-317B3.2 | | 9.54 | 0.046077602 | CB |
| IGHJ4 | immunoglobulin heavy joining 4 | 9.45 | 0.013184478 | CB |
| IGHGP | immunoglobulin heavy constant gamma P (non-functio [symbol] | 9.34 | 0.039288394 | CB |
| OR10G2 | olfactory receptor, family 10, subfamily 6, member 2 | 9.09 | 0.012125647 | CB |
| IGHE | immunoglobulin heavy constant epsilon | 8.83 | 0.027654311 | CB |
| BMS1P5 | BMS1 pseudogene 5 | 8.65 | 0.002970807 | CB |
| CST1 | cystatin SN | 8.61 | 0.034334741 | CB |
| IGHV1-69 | immunoglobulin heavy variable 1-69 | 8.58 | 0.036189278 | CB |
| RP11-534L6.3 | | 8.5 | 0.021641065 | CB |
| PPP1R14BP2 | protein phosphatase 1, regulatory (inhibitor) subunit 1 [symbol] | 8.44 | 0.033515231 | CB |
| IGLV1-51 | immunoglobulin lambda variable 1-51 | 8.42 | 0.046210752 | CB |
| GCNT1P3 | glucosaminyl (N-acetyl) transferase 1, core 2 pseudoge [symbol] | 8.28 | 0.049216823 | CB |
| RP11-151G12.2 | | 8.26 | 0.015181716 | CB |
| RP4-620F22.3 | | 7.94 | 0.041659897 | CB |
| CA14 | carbonic anhydrase XIV | 7.83 | 0.014371388 | CB |
| IGLV3-19 | immunoglobulin lambda variable 3-19 | 7.8 | 0.049633437 | CB |
| SHD | Src homology 2 domain containing transforming protei [symbol] | 7.73 | 0.013031872 | CB |
| RP11-2A7A.3 | | 7.58 | 0.028426013 | CB |
| IGHV4OR15-8 | immunoglobulin heavy variable 4/OR15-8 (non-functio [symbol] | 7.55 | 0.035978444 | CB |
| CKMT1B | creatine kinase, mitochondrial 1B | 7.53 | 0.045512017 | CB |
| IGKV1-5 | immunoglobulin kappa variable 1-5 | 7.45 | 0.022360081 | CB |
| RP11-115A14.1 | | 7.43 | 0.017845056 | CB |
| RP11-445O16.2 | | 7.18 | 0.020838997 | CB |
| SLITRK2 | SLIT and NTRK-like family, member 2 | 7.16 | 0.008070194 | CB |
| IGKJ5 | immunoglobulin kappa joining 5 | 7.09 | 0.02643572 | CB |
| CTD-2235H24.2 | | 7.08 | 0.030005872 | CB |
| IGKC | immunoglobulin kappa constant | 7.07 | 0.028688235 | CB |
| TTC24 | tetratricopeptide repeat domain 24 | 7 | 0.04883796 | CB |
| IGHG3 | immunoglobulin heavy constant gamma 3 (G3m marke [symbol] | 7 | 0.017221389 | CB |
| LRRC26 | leucine rich repeat containing 26 | 6.92 | 0.045085282 | CB |
| hsa-mir-220b | | 6.88 | 0.014969485 | CB |
| IGHJ3 | immunoglobulin heavy joining 3 | 6.85 | 0.020251381 | CB |
| AC007050.17 | | 6.83 | 0.040865625 | CB |
| RP11-33B1.3 | | 6.75 | 0.045085282 | CB |
| IGKV3D-20 | immunoglobulin kappa variable 3D-20 | 6.74 | 0.033794271 | CB |
| IGKJ5 | immunoglobulin kappa joining 5 | 6.69 | 0.036339918 | CB |
| CHIT1 | chitinase 1 (chitotriosidase) | 6.68 | 0.017221389 | CB |
| IGKJ1 | immunoglobulin kappa joining 1 | 6.65 | 0.02643572 | CB |
| CTD-2026G6.1 | | 6.65 | 0.013184478 | CB |
| AC005041.17 | | 6.57 | 0.039259375 | CB |
| IGLV7-43 | immunoglobulin lambda variable 7-43 | 6.54 | 0.02199666 | CB |
| ADCYAP1 | adenylate cyclase activating polypeptide 1 (pituitary) | 6.5 | 0.043523654 | CB |
| TRBV3-1 | T cell receptor beta variable 3-1 | 6.49 | 0.008690964 | CB |
| DUSP9 | dual specificity phosphatase 9 | 6.49 | 0.015687171 | CB |
| PHGR1 | proline/histidine/glycine-rich 1 | 6.48 | 0.009782763 | CB |
| TBL1Y | transducin (beta)-like 1, Y-linked | 6.42 | 0.03086908 | CB |
| IGLV3-1 | immunoglobulin lambda variable 3-1 | 6.33 | 0.024742224 | CB |
| IGKJ2 | immunoglobulin kappa joining 2 | 6.32 | 0.024814077 | CB |
| RP5-1198E17.1 | | 6.24 | 0.048733769 | CB |
| AC096579.13 | | 6.1 | 0.026864606 | CB |
| PAX5 | paired box 5 | 6.08 | 0.04488914 | CB |
| AL928742.12 | | 5.98 | 0.043395953 | CB |
| IGHM | immunoglobulin heavy constant mu | 5.97 | 0.049100171 | CB |
| DNM1P42 | DNM 1 pseudogene 38 | 5.97 | 0.01373806 | CB |
| CPNE7 | copine VII | 5.96 | 0.018812935 | CB |
| IGHG2 | immunoglobulin heavy constant gamma 2 (G2m marke [symbol] | 5.91 | 0.02643572 | CB |
| RP11-1280I22.1 | | 5.9 | 0.02678915 | CB |
| CCL18 | chemokine (C-C motif) ligand 18 (pulmonary and activa [symbol] | 5.87 | 0.02643572 | CB |
| IGKV3-20 | immunoglobulin kappa variable 3-20 | 5.84 | 0.031363566 | CB |
| RP11-1166P10.5 | | 5.83 | 0.020177433 | CB |
| RP11-17M16.1 | | 5.77 | 0.026146007 | CB |
| TRAV6 | T cell receptor alpha variable 6 | 5.73 | 0.027230317 | CB |
| IGJ | | 5.67 | 0.031092971 | CB |
| CD5L | CD5 molecule-like | 5.66 | 0.017111073 | CB |
| TRAV38-2DV8 | T cell receptor alpha variable 38-2/delta variable 8 | 5.64 | 0.017111073 | CB |
| TRAV1-1 | T cell receptor alpha variable 1-1 | 5.62 | 0.012652237 | CB |
| PAICSP3 | phosphoribosylaminoimidazole carboxylase, phosphori [symbol] | 5.61 | 0.022042996 | CB |
| RP11-367J7.3 | | 5.52 | 0.024542839 | CB |
| IGLC1 | immunoglobulin lambda constant 1 (Mcg marker) | 5.41 | 0.024328524 | CB |
| SLC12A3 | solute carrier family 12 (sodium/chloride transporter), [symbol] | 5.4 | 0.021083625 | CB |
| IGLL5 | immunoglobulin lambda-like polypeptide 5 | 5.37 | 0.022360081 | CB |
| TRBV11-2 | T cell receptor beta variable 11-2 | 5.35 | 0.019899858 | CB |
| CTB-193M12.3 | | 5.28 | 0.020059443 | CB |
| IGHV3-72 | immunoglobulin heavy variable 3-72 | 5.2 | 0.014711317 | CB |
| HS3ST2 | heparan sulfate (glucosamine) 3-O-sulfotransferase 2 | 5.19 | 0.015742933 | CB |
| FOXB1 | forkhead box B1 | 5.06 | 0.047375038 | CB |
| IGHV3-7 | immunoglobulin heavy variable 3-7 | 5.04 | 0.0422412 | CB |
| IGLJ1 | immunoglobulin lambda joining 1 | 5.02 | 0.027138346 | CB |
| SUSD5 | sushi domain containing 5 | 4.93 | 0.015742933 | CB |
| IRX2 | iroquois homeobox 2 | 4.91 | 0.045478051 | CB |
| TRAV12-1 | T cell receptor alpha variable 12-1 | 4.89 | 0.015921419 | CB |
| PTGDS | prostaglandin D2 synthase 21 kDa (brain) | 4.83 | 0.024328524 | CB |
| GOLGA7B | golgin A7 family, member B | 4.8 | 0.02052367 | CB |
| TCL1A | T-cell leukemia/lymphoma 1A | 4.8 | 0.047209651 | CB |
| RP11-731F5.2 | | 4.74 | 0.031092971 | CB |
| IGHV3-53 | immunoglobulin heavy variable 3-53 | 4.71 | 0.04213223 | CB |
| IGLV4-69 | immunoglobulin lambda variable 4-69 | 4.68 | 0.014251282 | CB |
| SIGLEC8 | sialic acid binding Ig-like lectin 8 | 4.66 | 0.003059735 | CB |
| IGHJ5 | immunoglobulin heavy joining 5 | 4.65 | 0.047474072 | CB |
| RP11-798M19.3 | | 4.63 | 0.032504427 | CB |
| RP11-615J4.4 | | 4.62 | 0.02875596 | CB |
| RP11-236F9.5 | | 4.62 | 0.012297912 | CB |
| FCRL3 | Fc receptor-like 3 | 4.59 | 0.018812935 | CB |
| TRAV8-3 | T cell receptor alpha variable 8-3 | 4.55 | 0.024375591 | CB |
| IL19 | interleukin 19 | 4.5 | 0.026302018 | CB |
| ZBP1 | Z-DNA binding protein 1 | 4.5 | 0.024814077 | CB |
| IGLV6-57 | immunoglobulin lambda variable 6-57 | 4.49 | 0.046776361 | CB |
| AL109761.5 | | 4.48 | 0.008747607 | CB |
| IGKV1-16 | immunoglobulin kappa variable 1-16 | 4.45 | 0.017656956 | CB |
| FCRL1 | Fc receptor-like 1 | 4.44 | 0.024742224 | CB |
| RP11-160A9.2 | | 4.43 | 0.006059865 | CB |
| RP11-96D1.5 | | 4.43 | 0.039680708 | CB |
| TUBB4A | tubulin, beta 4A class IVa | 4.42 | 0.02643572 | CB |
| IGHA1 | immunoglobulin heavy constant alpha 1 | 4.41 | 0.024328524 | CB |
| RP11-46D6.1 | | 4.38 | 0.024728355 | CB |
| FAM159A | family with sequence similarity 159, member A | 4.33 | 0.045512017 | CB |
| CD79A | CD79a molecule, immunoglobulin-associated alpha | 4.33 | 0.02643572 | CB |
| XXbac-B476C20.10 | | 4.33 | 0.00890364 | CB |
| AC013444.1 | | 4.32 | 0.009968933 | CB |
| CTD-3193O13.2 | | 4.29 | 0.035096859 | CB |
| IGHJ6 | immunoglobulin heavy joining 6 | 4.26 | 0.015703623 | CB |
| NSG1 | neuron specific gene family member 1 | 4.25 | 0.045638568 | CB |
| UGT2B17 | UDP glucuronosyltransferase 2 family, polypeptide B17 | 4.21 | 0.039234101 | CB |
| RUNX3 | runt-related transcription factor 3 | 4.2 | 0.003059735 | CB |
| MS4A1 | membrane-spanning 4-domains, subfamily A, member [symbol] | 4.2 | 0.042370292 | CB |
| TRBV7-8 | T cell receptor beta variable 7-8 | 4.15 | 0.003333637 | CB |
| C5orf38 | | 4.11 | 0.038479039 | CB |
| AC098850.4 | | 4.11 | 0.049100171 | CB |
| CD52 | CD52 molecule | 4.1 | 0.036386177 | CB |
| TRBV19 | T cell receptor beta variable 19 | 4.1 | 0.014762458 | CB |
| RP11-752G15.3 | | 4.09 | 0.007736506 | CB |
| ADAMDEC1 | ADAM-like, decysin 1 | 4.04 | 0.026864606 | CB |
| HM13-IT1 | HM13 intronic transcript 1 (non-protein coding) | 4.02 | 0.008747607 | CB |
| FAIM3 | Fas apoptotic inhibitory molecule 3 | 4 | 0.001257413 | CB |
| TRAV8-2 | T cell receptor alpha variable 8-2 | 3.98 | 0.018415671 | CB |
| IGHD | immunoglobulin heavy constant delta | 3.97 | 0.019304276 | CB |
| CDH3 | cadherin 3, type 1, P-cadherin (placental) | 3.96 | 0.031092971 | CB |
| IGKV1D-13 | immunoglobulin kappa variable 1D-13 | 3.93 | 0.041968771 | CB |
| TRBV6-5 | T cell receptor beta variable 6-5 | 3.92 | 0.022828147 | CB |
| GAPDHP66 | glyceraldehyde-3-phosphate dehydrogenase pseudoge [symbol] | 3.91 | 0.028010999 | CB |
| AC110615.1 | | 3.9 | 0.015372603 | CB |
| TRAV12-2 | T cell receptor alpha variable 12-2 | 3.89 | 0.008789839 | CB |
| 3-Sep | septin 3 | 3.89 | 0.028688235 | CB |
| RP11-444D3.1 | | 3.83 | 0.026538797 | CB |
| IL12A | interleukin 12A (natural killer cell stimulatory factor 1, [symbol] | 3.8 | 0.000890132 | CB |
| RP11-166B2.3 | | 3.8 | 0.016120921 | CB |
| POU2AF1 | POU class 2 associating factor 1 | 3.74 | 0.02287369 | CB |
| AL137072.1 | | 3.72 | 0.040458113 | CB |
| RP11-197M22.2 | | 3.72 | 0.003591886 | CB |
| RP11-304L20.1 | | 3.69 | 0.042281808 | CB |
| KLHDC7B | kelch domain containing 7B | 3.69 | 0.042370292 | CB |
| RP11-553K8.3 | | 3.64 | 0.007203049 | CB |
| FCRL2 | Fc receptor-like 2 | 3.62 | 0.033852011 | CB |
| IGKV3-15 | immunoglobulin kappa variable 3-15 | 3.61 | 0.042339272 | CB |
| PLA2G2D | phospholipase A2, group IID | 3.59 | 0.011547361 | CB |
| TRBV20-1 | T cell receptor beta variable 20-1 | 3.58 | 0.01262029 | CB |
| TRDV1 | T cell receptor delta variable 1 | 3.58 | 0.044163971 | CB |
| IL24 | interleukin 24 | 3.54 | 0.002180177 | CB |
| RASSF6 | Ras association (RalGDS/AF-6) domain family member [symbol] | 3.52 | 0.034369721 | CB |
| RP3-323N1.2 | | 3.5 | 0.006129994 | CB |
| TRAV2 | T cell receptor alpha variable 2 | 3.5 | 0.012569126 | CB |
| ZNF831 | zinc finger protein 831 | 3.5 | 0.029054717 | CB |
| AC004906.3 | | 3.48 | 0.033880884 | CB |
| Z97634.5 | | 3.44 | 0.020435079 | CB |
| USP43 | ubiquitin specific peptidase 43 | 3.42 | 0.036189278 | CB |
| IGHV1-3 | immunoglobulin heavy variable 1-3 | 3.41 | 0.004030313 | CB |
| KCNIP3 | Kv channel interacting protein 3, calsenilin | 3.38 | 0.003059735 | CB |
| CUX2 | cut-like homeobox 2 | 3.37 | 0.022828147 | CB |
| AC013264.2 | | 3.36 | 0.025869773 | CB |
| ICOS | inducible T-cell co-stimulator | 3.36 | 0.02683945 | CB |
| TRBV29-1 | T cell receptor beta variable 29-1 | 3.36 | 0.028975593 | CB |
| RP11-351I21.6 | | 3.36 | 0.02643572 | CB |
| WNT10A | wingless-type MMTV integration site family, member 1 [symbol] | 3.35 | 0.039148946 | CB |
| FCRLA | Fc receptor-like A | 3.34 | 0.033656613 | CB |
| TIGIT | T cell immunoreceptor with Ig and ITIM domains | 3.33 | 0.033656613 | CB |
| AC009499.1 | | 3.32 | 0.03076835 | CB |
| TRBV4-1 | T cell receptor beta variable 4-1 | 3.28 | 0.041968771 | CB |
| HLA-DQA1 | major histocompatiblity complex, class II, DQ alpha 1 | 3.26 | 0.033656613 | CB |
| DNTT | DNA nucleotidylexotransferase | 3.25 | 0.016753819 | CB |
| GNG4 | guanine nucleotide binding protein (G protein), gamma [symbol] | 3.24 | 0.029028339 | CB |
| RP11-641C17.4 | | 3.23 | 0.016475123 | CB |
| CD72 | CD72 molecule | 3.23 | 0.018812935 | CB |
| ST8SIA6 | ST8 alpha-N-acetyl-neuraminide alpha-2,8-siafyltransfe [symbol] | 3.23 | 0.042370292 | CB |
| KIAA0125 | KIAA0125 | 3.23 | 0.033698056 | CB |
| PTCRA | pre T-cell antigen receptor alpha | 3.22 | 0.026404029 | CB |
| UBASH3A | ubiquitin associated and SH3 domain containing A | 3.22 | 0.016408594 | CB |
| PYHIN1 | pyrin and HIN domain family, member 1 | 3.19 | 0.011925792 | CB |
| EOMES | eomesodermin | 3.19 | 0.001902564 | CB |
| CD6 | CD6 molecule | 3.18 | 0.011925792 | CB |
| RP11-74K11.1 | | 3.18 | 0.04213223 | CB |
| LILRA4 | leukocyte immunoglobulin-like receptor, subfamily A ( [symbol] | 3.18 | 0.015742933 | CB |
| CD3G | CD3g molecule, gamma (CD3-TCR complex) | 3.17 | 0.010595239 | CB |
| TRAV3 | T cell receptor alpha variable 3 (gene/pseudogene) | 3.17 | 0.04488914 | CB |
| OR6S1 | olfactory receptor, family 6, subfamily S, member 1 | 3.16 | 0.033574388 | CB |
| IGKV3D-15 | immunoglobulin kappa variable 3D-15 (gene/pseudoge [symbol] | 3.15 | 0.02683945 | CB |
| S1PR4 | sphingosine-1-phosphate receptor 4 | 3.14 | 0.04883796 | CB |
| MNX1 | motor neuron and pancreas homeobox 1 | 3.12 | 0.037825665 | CB |
| LY9 | lymphocyte antigen 9 | 3.1 | 0.021105252 | CB |
| RP11-481A20.10 | | 3.08 | 0.023509294 | CB |
| RP11-80A15.1 | | 3.08 | 0.01692508 | CB |
| RP1-127D3.4 | | 3.06 | 0.012666399 | CB |
| RP11-290F20.3 | | 3.06 | 0.011925792 | CB |
| AL139819.1 | | 3.05 | 0.013811098 | CB |
| ACTL8 | actin-like 8 | 3.04 | 0.0100951 | CB |
| ZBED2 | zinc finger, BED-type containing 2 | 3.03 | 0.004030313 | CB |
| ITK | IL2-inducible T-cell kinase | 3.03 | 0.049100171 | CB |
| TRAV1-2 | T cell receptor alpha variable 1-2 | 3.02 | 0.009442924 | CB |
| SLAMF6 | SLAM family member 6 | 3.01 | 0.010840451 | CB |
| RPSAP53 | ribosomal protein SA pseudogene 53 | 3.01 | 0.028688235 | CB |
| RP11-481A20.11 | | 2.99 | 0.009241778 | CB |
| IDO1 | indoleamine 2,3-dioxygenase 1 | 2.99 | 0.021105252 | CB |
| RP11-195C7.3 | | 2.98 | 0.029602879 | CB |
| HSH2D | hematopoietic SH2 domain containing | 2.98 | 0.028975593 | CB |
| AC002306.1 | | 2.97 | 0.021494609 | CB |
| RP11-379H8.1 | | 2.96 | 0.036500639 | CB |
| CRYM | crystallin, mu | 2.96 | 0.045478051 | CB |
| CXorf57 | | 2.96 | 0.017221389 | CB |
| KRT27 | keratin 27 | 2.95 | 0.011193228 | CB |
| SNRPFP1 | small nuclear ribonucleoprotein polypeptide F pseudog [symbol] | 2.95 | 0.047209651 | CB |
| IGLV2-11 | immunoglobulin lambda variable 2-11 | 2.95 | 0.028975593 | CB |
| RP11-293F5.4 | | 2.9 | 0.030369085 | CB |
| SPOCK2 | sparc/osteonectin, cwcv and kazal-like domains proteo [symbol] | 2.9 | 0.028688235 | CB |
| C11orf41 | | 2.9 | 0.02052367 | CB |
| CD7 | CD7 molecule | 2.9 | 0.036386177 | CB |
| FSD1 | fibronectin type III and SPRY domain containing 1 | 2.9 | 0.031391184 | CB |
| TMEM191A | transmembrane protein 191A (pseudogene) | 2.9 | 0.026864606 | CB |
| RP11-292F22.3 | | 2.89 | 0.032737245 | CB |
| RAB19 | RAB19, member RAS oncogene family | 2.88 | 0.033351877 | CB |
| PRF1 | perforin 1 (pore forming protein) | 2.88 | 0.015742933 | CB |
| RP11-332O19.3 | | 2.85 | 0.007014254 | CB |
| CTC-422A18.2 | | 2.84 | 0.040365068 | CB |
| NR4A3 | nuclear receptor subfamily 4, group A, member 3 | 2.84 | 0.004785714 | CB |
| CD3D | CD3d molecule, delta (CD3-TCR complex) | 2.84 | 0.014371388 | CB |
| SCML1 | sex comb on midleg-like 1 (Drosophila) | 2.84 | 0.031092971 | CB |
| THEMIS | thymocyte selection associated | 2.83 | 0.045478051 | CB |
| HSD11B1 | hydroxysteroid (11-beta) dehydrogenase 1 | 2.81 | 0.028688235 | CB |
| IL28RA | interferon, lambda receptor 1 | 2.8 | 0.010595239 | CB |
| GNLY | granulysin | 2.8 | 0.014371388 | CB |
| RP11-429J17.6 | | 2.8 | 0.014371388 | CB |
| LCK | lymphocyte-specific protein tyrosine kinase | 2.79 | 0.015079112 | CB |
| RPL5P32 | ribosomal protein L5 pseudogene 32 | 2.78 | 0.019540488 | CB |
| AC023490.1 | | 2.78 | 0.013100955 | CB |
| RP11-564A8.4 | | 2.77 | 0.000583885 | CB |
| RP11-220D10.1 | | 2.77 | 0.017559073 | CB |
| TNFRSF11B | tumor necrosis factor receptor superfamily, member 1 [symbol] | 2.75 | 0.042339272 | CB |
| ATP1A3 | ATPase, Na+/K+ transporting, alpha 3 polypeptide | 2.73 | 0.024328524 | CB |
| IKZF3 | IKAROS family zinc finger 3 (Aiolos) | 2.72 | 0.014371388 | CB |
| C16orf54 | | 2.7 | 0.029054717 | CB |
| AC008984.2 | | 2.7 | 0.004072591 | CB |
| CXCL13 | chemokine (C-X-C motif) ligand 13 | 2.69 | 0.019434049 | CB |
| RP11-599J14.2 | | 2.67 | 0.039288394 | CB |
| TRAV5 | T cell receptor alpha variable 5 | 2.67 | 0.033575171 | CB |
| RP11-566K11.2 | | 2.67 | 0.035185369 | CB |
| CD96 | CD96 molecule | 2.66 | 0.042339272 | CB |
| C8orf80 | | 2.66 | 0.036500639 | CB |
| TNFRSF13B | tumor necrosis factor receptor super-family, member 1 [symbol] | 2.65 | 0.04352477 | CB |
| TRAV13-1 | T cell receptor alpha variable 13-1 | 2.64 | 0.024662434 | CB |
| EIF4BP9 | eukaryotic translation initiation factor 48 pseudogene [symbol] | 2.64 | 0.007991506 | CB |
| PTPRCAP | protein tyrosine phosphatase, receptor type, C-associa [symbol] | 2.62 | 0.02052367 | CB |
| SLC24A4 | solute carrier family 24 (sodium/potassium/calcium ex [symbol] | 2.62 | 0.028688235 | CB |
| GZMH | granzyme H (cathepsin G-like 2, protein h-CCPX) | 2.61 | 0.045512017 | CB |
| TBX21 | T-box 21 | 2.61 | 0.021040385 | CB |
| SLC22A18AS | solute carrier family 22 (organic cation transporter), m [symbol] | 2.59 | 0.036530778 | CB |
| LINC00426 | long intergenic non-protein coding RNA 426 | 2.59 | 0.009381276 | CB |
| HSBP1L1 | heat shock factor binding protein 1-like 1 | 2.58 | 0.001681771 | CB |
| TRAC | T cell receptor alpha constant | 2.57 | 0.013100955 | CB |
| MTFP1 | mitochondrial fission process 1 | 2.57 | 0.00657893 | CB |
| 1-Sep | septin 1 | 2.55 | 0.039288394 | CB |
| IL6R | interleukin 6 receptor | 2.54 | 0.049100171 | CB |
| RP11-61I13.2 | | 2.54 | 0.017952081 | CB |
| CATSPERB | catsper channel auxiliary subunit beta | 2.53 | 0.022360081 | CB |
| LAG3 | lymphocyte-activation gene 3 | 2.52 | 0.028688235 | CB |
| LTB | lymphotoxin beta (TNF superfamily, member 3) | 2.51 | 0.036386177 | CB |
| GZMB | granzyme B (granzyme 2, cytotoxic T-lymphocyte-asso [symbol] | 2.51 | 0.026688235 | CB |
| TRBV4-2 | T cell receptor beta variable 4-2 | 2.49 | 0.048226887 | CB |
| C14orf182 | | 2.49 | 0.009675088 | CB |
| HS3ST3B1 | heparan sulfate (glucosamine) 3-O-sulfotransferase 3B [symbol] | 2.49 | 0.045512017 | CB |
| CTD-2562J17.3 | | 2.48 | 0.002755485 | CB |
| RP11-630I5.1 | | 2.46 | 0.047209651 | CB |
| BEND4 | BEN domain containing 4 | 2.46 | 0.003304376 | CB |
| IL12RB1 | interleukin 12 receptor, beta 1 | 2.45 | 0.045638568 | CB |
| KLHL6 | kelch-like family member 6 | 2.44 | 0.049100171 | CB |
| RP11-126K1.8 | | 2.43 | 0.021734161 | CB |
| PARP15 | poly (ADP-ribose) polymerase family, member 15 | 2.43 | 0.02643572 | CB |
| RP13-554M15.6 | | 2.43 | 0.020709572 | CB |
| TMEM238 | transmembrane protein 238 | 2.43 | 0.011925792 | CB |
| AC104698.1 | | 2.41 | 0.026170063 | CB |
| FASLG | Fas ligand (TNF superfamily, member 6) | 2.4 | 0.028975593 | CB |
| GYLTL1B | glycosyltransferase-like 1B | 2.39 | 0.029054717 | CB |
| IL29 | interferon, lambda 1 | 2.38 | 0.030404894 | CB |
| GZMK | granzyme K (granzyme 3; tryptase ▮) | 2.37 | 0.024742224 | CB |
| FHOD3 | formin homology 2 domain containing 3 | 2.37 | 0.042370292 | CB |
| PRUNE2 | prune homolog 2 (Drosophila) | 2.36 | 0.042370292 | CB |
| FXYD5 | FXYD domain containing ion transport regulator 5 | 2.35 | 0.002724323 | CB |
| CTLA4 | cytotoxic T-lymphocyte-associated protein 4 | 2.34 | 0.024814077 | CB |
| ATP13A4-AS1 | ATP13A4 antisense RNA 1 | 2.34 | 0.015249492 | CB |
| RP11-44K6.4 | | 2.34 | 0.036500639 | CB |
| PFKP | phosphofructokinase, platelet | 2.34 | 0.011925792 | CB |
| TRAV8-4 | T cell receptor alpha variable 8-4 | 2.34 | 0.02857165 | CB |
| RP11-1094M14.3 | | 2.34 | 0.049100171 | CB |
| ZMYND12 | zinc finger, MYND-type containing 12 | 2.33 | 0.024328524 | CB |
| TRBC2 | T cell receptor beta constant 2 | 2.33 | 0.011925792 | CB |
| RP11-1396O13.20 | | 2.32 | 0.024328524 | CB |
| TRBV5-5 | T cell receptor beta variable 5-5 | 2.32 | 0.049758592 | CB |
| TRAT1 | T cell receptor associated transmembrane adaptor 1 | 2.31 | 0.045478051 | CB |
| CHAC1 | ChaC, cation transport regulator homolog 1 (E. coli) | 2.31 | 0.011925792 | CB |
| SLC1A4 | solute carrier famiy 1 (glutamate/neutral amino acid t [symbol] | 2.3 | 0.013100955 | CB |
| RPS3AP34 | ribosomal protein S3a pseudogene 34 | 2.3 | 0.018812935 | CB |
| RP11-982M15.6 | | 2.3 | 0.036386177 | CB |
| JAKMIP1 | jan us kinase and microtubule interacting protein 1 | 2.29 | 0.033852011 | CB |
| CPEB1 | cytoplasmic polyadenylation element binding protein 1 | 2.29 | 0.039288394 | CB |
| AC104820.2 | | 2.28 | 0.002764257 | CB |
| KCNMA1 | potassium large conductance calcium-activated channe | 2.28 | 0.022360081 | CB |
| RP11-93B14.6 | | 2.28 | 0.022437406 | CB |
| NCF1B | neutrophil cytosolic factor 1B pseudogene | 2.27 | 0.049100171 | CB |
| HLF | hepatic leukemia factor | 2.27 | 0.049100171 | CB |
| RPS6KA1 | ribosomal protein S6 kinase, 90 kDa, polypeptide 1 | 2.26 | 0.013100955 | CB |
| RP13-735L24.1 | | 2.26 | 0.03877208 | CB |
| TMEM108 | transmembrane protein 108 | 2.25 | 0.045638568 | CB |
| RP11-982M15.8 | | 2.25 | 0.049369302 | CB |
| IGFLR1 | IGF-like family receptor 1 | 2.24 | 0.036386177 | CB |
| TNIP3 | TNIFAIP3 interacting protein 3 | 2.23 | 0.031391184 | CB |
| RP11-443A13.3 | | 2.23 | 0.015742933 | CB |
| TRGV7 | T cell receptor gamma variable 7 (pseudogene) | 2.22 | 0.011590706 | CB |
| OIT3 | ancoprotein induced transcript 3 | 2.22 | 0.036500639 | CB |
| SLA2 | Src-like-adaptor 2 | 2.22 | 0.018812935 | CB |
| ZNF683 | zinc finger protein 683 | 2.21 | 0.045478051 | CB |
| SAMD5 | sterile alpha motif domain containing 5 | 2.21 | 0.029054717 | CB |
| GPR174 | G protein-coupled receptor 174 | 2.21 | 0.031391184 | CB |
| SASH3 | SAM and SH3 domain containing 3 | 2.21 | 0.022360081 | CB |
| LAX1 | lymphocyte transmembrane adaptor 1 | 2.2 | 0.033656613 | CB |
| KCNAB2 | potassium voltage-gated channel, shaker related subfa [symbol] | 2.19 | 0.003430441 | CB |
| HIST1H2BG | histone cluster 1, H2bg | 2.19 | 0.039288394 | CB |
| BIK | BCL2-interacting killer (apoptosis-inducing) | 2.19 | 0.015079112 | CB |
| CD2 | CD2 molecule | 2.18 | 0.033656613 | CB |
| RP11-10G12.1 | | 2.17 | 0.008391308 | CB |
| FTH1P24 | ferritin, heavy polypeptide 1 pseudogene 24 | 2.17 | 0.041434796 | CB |
| PLP2 | proteolipid protein 2 (colonic epithelium-enriched) | 2.17 | 0.003839527 | CB |
| SLC34A1 | solute carrier family 34 (type II sodium/phosphate cont [symbol] | 2.16 | 0.041434796 | CB |
| RP11-771F20.1 | | 2.16 | 0.031092971 | CB |
| IFITM5 | interferon induced transmembrane protein 5 | 2.15 | 0.036189278 | CB |
| RP11-390B4.3 | | 2.13 | 0.004290131 | CB |
| CXCR3 | chemokine (C-X-C motif) receptor 3 | 2.13 | 0.039288394 | CB |
| TRGC1 | T cell receptor gamma constant 1 | 2.12 | 0.039316473 | CB |
| CD3E | CD3e molecule, epsilon (CD3-TCR complex) | 2.12 | 0.012666399 | CB |
| SIT1 | signaling threshold regulating transmembrane adaptor | 2.11 | 0.033852011 | CB |
| NKG7 | natural killer cell granule protein 7 | 2.11 | 0.024328524 | CB |
| C1orf51 | | 2.1 | 0.028588235 | CB |
| ABCD2 | ATP binding cassette, sub-family D (ALD), member 2 | 2.1 | 0.028688235 | CB |
| TNIK | TRAF2 and NCK interacting kinase | 2.09 | 0.045638568 | CB |
| DNAJC6 | DnaJ (Hsp40) homolog, subfamily C, member 6 | 2.08 | 0.036386177 | CB |
| SLCO4A1 | solute carrier organic anion transporter family, membe [symbol] | 2.08 | 0.031092971 | CB |
| HSD3B7 | hydroxy-delta-5-steroid dehydrogenase, 3 beta- and st [symbol] | 2.07 | 0.004290131 | CB |
| CR2 | complement component (3d/Epstein Barr virus) recept [symbol] | 2.06 | 0.042339272 | CB |
| TC2N | tandem C2 domains, nuclear | 2.06 | 0.045638568 | CB |
| IL12RB2 | interleukin 12 receptor, beta 2 | 2.05 | 0.028688235 | CB |
| RGS14 | regulator of G-protein signaling 14 | 2.05 | 0.005329798 | CB |
| CLEC19A | C-type lectin domain family 19, member A | 2.04 | 0.041659897 | CB |
| MYO1G | myosin IG | 2.03 | 0.049100171 | CB |
| RHBDL1 | rhomboid, veinlet-like 1 (Drosophia) | 2.03 | 0.024328524 | CB |
| CD19 | CD19 molecule | 2.03 | 0.028688235 | CB |
| TJP3 | tight junction protein 3 | 2.03 | 0.021083625 | CB |
| PIK3CD | phosphatidylinositol-4,5-bisphosphate 3-kinase, catalyt [symbol] | 2.02 | 0.022360081 | CB |
| DHRS9 | dehydrogenase/reductase (SDR family) member 9 | 2.02 | 0.008917729 | CB |
| RP3-453I5.2 | | 2.02 | 0.031835936 | CB |
| ITGAL | integrin, alpha L (antigen CD11A (p180), lymphocyte fu [symbol] | 2.02 | 0.024328524 | CB |
| NLRC5 | NLR family, CARD domain containing 5 | 2.02 | 0.009839457 | CB |
| LPIN3 | lipin 3 | 2.02 | 0.036386177 | CB |
| NLGN4Y | neuroligin 4, Y-linked | 2.02 | 0.036386177 | CB |
| DTHD1 | death domain containing 1 | 2.01 | 0.039347886 | CB |
| RP11-351I21.7 | | 2.01 | 0.033656613 | CB |
| FOXH1 | forkhead box H1 | 2.01 | 0.038385003 | CB |
| ZNF296 | zinc finger protein 296 | 2.01 | 0.039288394 | CB |

[symbol]

indicates data missing or illegible when filed

TABLE 3
Genes enriched in TCGA Xq28-CGA-high/low expression samples

| Gene | Gene_Name | Fold_Change | P-value | Group |
|---|---|---|---|---|
| ?\|100133144 | | 2.57 | 4.18E−06 | Xq28-CGA high |
| ?\|100134869 | | 2.59 | 7.35E−09 | Xq28-CGA high |
| ?\|340602 | | 2.1 | 2.38E−05 | Xq28-CGA high |
| ?\|391343 | | 3.38 | 2.94E−15 | Xq28-CGA high |
| AACSL\|729522 | acetoacetyl-CoA synthetase pseudogene 1 | 5.79 | 1.38E−14 | Xq28-CGA high |
| ABCA8\|10351 | ATP-binding cassette, sub-family A (ABC1), mem [symbol] | 11.7 | 2.59E−05 | Xq28-CGA high |
| ABHD1\|84696 | abhydrolase domain containing 1 | 2.2 | 9.03E−07 | Xq28-CGA high |
| ACCN4\|55515 | acid-sensing (proton-gated) ion channel family m [symbol] | 2.68 | 3.92E−05 | Xq28-CGA high |
| ACOT11\|26027 | acyl-CoA thioesterase 11 | 2.01 | 9.52E−07 | Xq28-CGA high |
| ACSM3\|6296 | acyl-CoA synthetase medium-chain family memb [symbol] | 2.11 | 1.45E−04 | Xq28-CGA high |
| ADAM21P1\|145241 | ADAM metallopeptidase domain 21 pseudogene [symbol] | 2.04 | 1.45E−06 | Xq28-CGA high |
| ADAM21\|8747 | ADAM metallopeptidase domain 21 | 2.16 | 7.13E−05 | Xq28-CGA high |
| ADAMTS20\|80070 | ADAM metallopeptidase with thrombospondin t [symbol] | 8.83 | 5.40E−18 | Xq28-CGA high |
| ADAMTS3\|9508 | ADAM metallopeptidase with thrombospondin t [symbol] | 3.28 | 8.96E−05 | Xq28-CGA high |
| AGBL4\|84871 | ATP/GTP binding protein-like 4 | 2.86 | 5.45E−07 | Xq28-CGA high |
| AKAP6\|9472 | A kinase (PRKA) anchor protein 6 | 5.2 | 1.25E−09 | Xq28-CGA high |
| ALDH1L1\|10840 | aldehyde dehydrogenase 1 family, member L1 | 4.51 | 1.49E−05 | Xq28-CGA high |
| ALDH8A1\|64577 | aldehyde dehydrogenase 8 family, member A1 | 2.29 | 8.54E−09 | Xq28-CGA high |
| ANGPT1\|284 | angiopoietin 1 | 16.65 | 7.33E−21 | Xq28-CGA high |
| ANKFN1\|162282 | ankyrin-repeat and fibronectin type III domain c [symbol] | 3.97 | 2.53E−06 | Xq28-CGA high |
| ANKRD20A3\|441425 | ankyrin repeat domain 20 family, member A3 | 2.03 | 2.97E−05 | Xq28-CGA high |
| ANKRD20A4\|728747 | ankyrin repeat domain 20 family, member A4 | 2.02 | 4.49E−05 | Xq28-CGA high |
| ANKRD20B\|729171 | ankyrin repeat domain 20 family, member A8, p [symbol] | 3.12 | 1.46E−13 | Xq28-CGA high |
| ANKRD43\|134548 | ankyrin repeat domain family member A | 2.23 | 6.52E−07 | Xq28-CGA high |
| ANKRD45\|339416 | ankyrin repeat domain 45 | 8.13 | 7.90E−12 | Xq28-CGA high |
| ANKRD7\|56311 | ankyrin repeat domain 7 | 2.62 | 2.73E−06 | Xq28-CGA high |
| ANO3\|63982 | anoctamin 3 | 10.84 | 3.30E−15 | Xq28-CGA high |
| ANO4\|121601 | anoctamin 4 | 6.62 | 7.25E−07 | Xq28-CGA high |
| ANO5\|203859 | anoctamin 5 | 6.36 | 6.54E−05 | Xq28-CGA high |
| ARHGEF35\|445328 | Rho guanine nucleotide exchange factor (GEF) 3 [symbol] | 3.74 | 5.02E−07 | Xq28-CGA high |
| ARHGEF5\|7984 | Rho guanine nucleotide exchange factor (GEF) 5 | 2.61 | 1.31E−04 | Xq28-CGA high |
| ART3\|419 | ADP-ribosyltransferase 3 | 3.07 | 1.68E−05 | Xq28-CGA high |
| ASB4\|51666 | ankyrin repeat and SOCS box containing 4 | 9.37 | 2.34E−10 | Xq28-CGA high |
| ASCL1\|429 | achaete-scute family bHLH transcription factor 1 [symbol] | 6.46 | 3.94E−10 | Xq28-CGA high |
| ATP1B1\|481 | ATPase, Na+/K+ transporting, beta 1 polypeptide [symbol] | 2.33 | 2.89E−06 | Xq28-CGA high |
| ATP1B2\|482 | ATPase, Na+/K+ transporting, beta 2 polypeptide [symbol] | 6.39 | 4.48E−07 | Xq28-CGA high |
| AURKAPS1\|6791 | aurora kinase A pseudogene 1 | 2.27 | 9.37E−18 | Xq28-CGA high |
| AZGP1\|563 | alpha-2-glycoprotein 1, zinc-binding | 3.89 | 2.93E−09 | Xq28-CGA high |
| B3GALNT1\|8706 | beta-1,3-N-acetylgalactosaminyltransferase 1 (g [symbol] | 3.07 | 9.52E−07 | Xq28-CGA high |
| BAAT\|570 | bile acid CoA: amino add N-acyltransferase (glyc [symbol] | 4.88 | 1.10E−04 | Xq28-CGA high |
| BAGE2\|85319 | B melanoma antigen family, member 2 | 6.61 | 6.56E−19 | Xq28-CGA high |
| BAGE\|574 | B melanoma antigen | 322.69 | 8.19E−30 | Xq28-CGA high |
| BDKRB1\|623 | bradykinin receptor B1 | 2.02 | 4.21E−06 | Xq28-CGA high |
| C10orf62\|414157 | | 2.32 | 1.91E−07 | Xq28-CGA high |
| C11orf9\|745 | | 3.02 | 7.92E−07 | Xq28-CGA high |
| C12orf56\|115749 | | 12.21 | 2.93E−08 | Xq28-CGA high |
| C17orf47\|284083 | | 2.01 | 5.46E−06 | Xq28-CGA high |
| C17orf57\|124989 | | 2.69 | 1.88E−09 | Xq28-CGA high |
| C18orf2\|56651 | | 135.15 | 1.52E−22 | Xq28-CGA high |
| C1QTNF3\|114899 | C1q and tumor necrosis factor related protein 3 | 3.69 | 6.53E−06 | Xq28-CGA high |
| C1orf114\|57821 | | 9.23 | 8.34E−11 | Xq28-CGA high |
| C1orf175\|374977 | | 2.41 | 4.24E−06 | Xq28-CGA high |
| C1orf88\|128344 | | 2.19 | 5.98E−05 | Xq28-CGA high |
| C21orf29\|54084 | | 2.29 | 2.18E−07 | Xq28-CGA high |
| C21orf90\|114043 | | 10.16 | 2.71E−11 | Xq28-CGA high |
| C22orf34\|348645 | | 6.07 | 2.01E−12 | Xq28-CGA high |
| C2orf61\|285051 | | 2.72 | 3.12E−05 | Xq28-CGA high |
| C2orf66\|401027 | | 3.05 | 2.73E−07 | Xq28-CGA high |
| C3orf20\|84077 | | 2.16 | 5.04E−05 | Xq28-CGA high |
| C3orf30\|152405 | | 6.01 | 1.77E−13 | Xq28-CGA high |
| C3orf50\|93556 | | 7.19 | 1.32E−04 | Xq28-CGA high |
| C3orf66\|677779 | | 10.47 | 7.19E−07 | Xq28-CGA high |
| C4orf19\|55286 | | 4.83 | 1.11E−07 | Xq28-CGA high |
| C4orf37\|285555 | | 2.87 | 1.39E−04 | Xq28-CGA high |
| C4orf6\|10141 | | 3.81 | 3.52E−05 | Xq28-CGA high |
| C5orf58\|133874 | | 3.33 | 4.42E−07 | Xq28-CGA high |
| C6orf164\|63914 | | 2.09 | 4.98E−06 | Xq28-CGA high |
| C7orf51\|222950 | | 2.06 | 9.21E−06 | Xq28-CGA high |
| C7orf71\|285941 | | 2.42 | 5.51E−05 | Xq28-CGA high |
| C8orf85\|441376 | | 4.73 | 2.02E−07 | Xq28-CGA high |
| CA6\|765 | carbonic anhydrase VI | 8.97 | 7.57E−05 | Xq28-CGA high |
| CABP4\|57010 | calcium tending protein 4 | 2.09 | 2.10E−05 | Xq28-CGA high |
| CABYR\|26256 | calcium binding tyrosine-(Y)-phosphorylation reg [symbol] | 2.12 | 9.54E−06 | Xq28-CGA high |
| CADM4\|199731 | cell adhesion molecule 4 | 2.43 | 2.24E−07 | Xq28-CGA high |
| CALCB\|797 | calcitonin-related polypeptide beta | 2.36 | 1.45E−04 | Xq28-CGA high |
| CAND2\|23066 | cullin-associated and neddylation-dissociated 2 [symbol] | 2.58 | 2.69E−05 | Xq28-CGA high |
| CASP12\|120329 | caspase 12 (gene/pseudogene) | 2.92 | 1.13E−04 | Xq28-CGA high |
| CCDC136\|64753 | coiled-coil domain containing 136 | 3.39 | 3.18E−10 | Xq28-CGA high |
| CCDC144A\|9720 | coiled-coil domain containing 144A | 2.57 | 4.08E−07 | Xq28-CGA high |
| CCDC144B\|284047 | coiled-coil domain containing 144B (pseudogene [symbol] | 2.21 | 1.80E−08 | Xq28-CGA high |
| CCDC144C\|348254 | coiled-coil domain containing 144C, pseudogene [symbol] | 2.08 | 5.83E−07 | Xq28-CGA high |
| CCDC160\|347475 | coiled-coil domain containing 160 | 5.56 | 2.95E−07 | Xq28-CGA high |
| CCDC46\|201134 | centrosomal protein 112 kDa | 2.21 | 2.31E−05 | Xq28-CGA high |
| CCR10\|2826 | chemokine (C-C motif) receptor 10 | 2.59 | 6.20E−05 | Xq28-CGA high |
| CDH12\|1010 | cadherin 12, type 2 (N-cadherin 2) | 2.06 | 1.18E−10 | Xq28-CGA high |
| CDH18\|1016 | cadherin 18, type 2 | 2.26 | 1.43E−08 | Xq28-CGA high |
| CDK14\|5218 | cyclin-dependent kinase 14 | 2.15 | 2.69E−05 | Xq28-CGA high |
| CDK6\|1021 | cyclin-dependent kinase 6 | 2.03 | 3.37E−09 | Xq28-CGA high |
| CFHR4\|10877 | complement factor H-related 4 | 3.93 | 3.25E−15 | Xq28-CGA high |
| CFI\|3426 | complement factor I | 2.32 | 6.77E−06 | Xq28-CGA high |
| CHML\|1122 | choroideremia-like (Rab escort protein 2) | 2.29 | 8.66E−10 | Xq28-CGA high |
| CHRM3\|1131 | cholinergic receptor, muscarinic 3 | 6.32 | 8.83E−09 | Xq28-CGA high |
| CLEC18B\|497190 | C-type lectin domain family 18, member B | 2.8 | 2.48E−07 | Xq28-CGA high |
| CLEC18C\|283971 | C-type lectin domain family 18, member C | 3.29 | 2.57E−06 | Xq28-CGA high |
| CLEC2L\|154790 | C-type lectin domain family 2, member L | 4.49 | 5.06E−10 | Xq28-CGA high |
| CLGN\|1047 | calmegin | 2.63 | 1.48E−04 | Xq28-CGA high |
| CMTM8\|152189 | CKLF-like MARVEL transmembrane domain cont [symbol] | 2.03 | 2.30E−06 | Xq28-CGA high |
| CNDP1\|84735 | carnosine dipeptidase 1 (metallopeptidase M20 | 10.44 | 2.04E−07 | Xq28-CGA high |
| CNR1\|1268 | cannabinoid receptor 1 (brain) | 5.5 | 1.89E−08 | Xq28-CGA high |
| COL11A2\|1302 | collagen, type XI, alpha 2 | 4.71 | 4.25E−06 | Xq28-CGA high |
| COL25A1\|84570 | collagen, type XXV, alpha 1 | 3.06 | 2.67E−05 | Xq28-CGA high |
| COL28A1\|340267 | collagen, type XXVIII, alpha 1 | 2.41 | 6.36E−07 | Xq28-CGA high |
| COX6A2\|1339 | cytochrome c oxidase subunit VIa polypeptide 2 | 24.16 | 9.07E−06 | Xq28-CGA high |
| CPS1\|1373 | carbamoyl-phosphate synthase 1, mitochondrial [symbol] | 2.14 | 5.82E−05 | Xq28-CGA high |
| CPT1C\|126129 | carnitine palmitoyltransferase 1C | 2.45 | 5.38E−08 | Xq28-CGA high |
| CSAG1\|158511 | chondrosarcoma associated gene 1 | 1195.25 | 2.66E−40 | Xq28-CGA high |
| CSAG2\|728461 | CSAG family, member 2 | 2834.71 | 1.71E−44 | Xq28-CGA high |
| CSAG3\|389903 | CSAG family, member 3 | 907.95 | 2.54E−40 | Xq28-CGA high |
| CSMD1\|64478 | CUB and Sushi multiple domains 1 | 65.06 | 9.87E−23 | Xq28-CGA high |
| CSNK1A1P\|161635 | casein kinase 1, alpha 1 pseudogene 1 | 2.79 | 1.12E−04 | Xq28-CGA high |
| CTAG1B\|1485 | cancer/testis antigen 1B | 2124.92 | 2.20E−23 | Xq28-CGA high |
| CTAG2\|30848 | cancer/testis antigen 2 | 3883.48 | 5.49E−22 | Xq28-CGA high |
| CTAGE4\|100128553 | CTAGE family, member 4 | 3.87 | 4.57E−08 | Xq28-CGA high |
| CTAGE6\|340307 | CTAGE family, member 6 | 2.55 | 1.66E−07 | Xq28-CGA high |
| CTAGE9\|643854 | CTAGE family, member 9 | 4.12 | 1.72E−08 | Xq28-CGA high |
| CUZD1\|50624 | CUB and zona pellucida-like domains 1 | 2.51 | 3.82E−07 | Xq28-CGA high |
| CXorf42\|158801 | | 2.95 | 1.11E−04 | Xq28-CGA high |
| CXorf48\|54967 | | 9.38 | 5.16E−17 | Xq28-CGA high |
| CXorf49B\|100132994 | | 2.41 | 1.66E−09 | Xq28-CGA high |
| CYP26A1\|1592 | cytochrome P450, family 26, subfamily A, polype [symbol] | 3.95 | 4.44E−08 | Xq28-CGA high |
| CYP2J2\|1573 | cytochrome P450, family 2, subfamily J, polypep [symbol] | 6.31 | 4.44E−11 | Xq28-CGA high |
| DCAF4L1\|285429 | DDB1 and CUL4 associated factor 4-like 1 | 2.41 | 2.05E−10 | Xq28-CGA high |
| DCAF4L2\|138009 | DDB1 and CUL4 associated factor 4-like 2 | 2.39 | 5.69E−07 | Xq28-CGA high |
| DCAF8L2\|347442 | DDB1 and CUL4 associated factor 8-like 2 | 3.37 | 1.15E−08 | Xq28-CGA high |
| DDIT4L\|115265 | DNA-damage-inducible transcript 4-like | 2.07 | 9.75E−05 | Xq28-CGA high |
| DDO\|8528 | D-aspartate oxidase | 6.08 | 6.90E−08 | Xq28-CGA high |
| DDX25\|29118 | DEAD (Asp-Glu-Ala-Asp) box helicase 25 | 3.43 | 3.83E−06 | Xq28-CGA high |
| DEPDC1\|55635 | DEP domain containing 1 | 2.07 | 8.23E−07 | Xq28-CGA high |
| DHDPSL\|112817 | 4-hydroxy-2-oxoglutarate aldolase 1 | 2.24 | 3.77E−06 | Xq28-CGA high |
| DHRS2\|10202 | dehydrogenase/reductase (SDR family) member | 3.49 | 1.53E−05 | Xq28-CGA high |
| DLX2\|1746 | distal-less homeobox 2 | 2.35 | 1.54E−05 | Xq28-CGA high |
| DMP1\|1758 | dentin matrix acidic phosphoprotein 1 | 2.14 | 2.47E−06 | Xq28-CGA high |
| DNAH2\|146754 | dynein, axonemal, heavy chain 2 | 9.72 | 3.37E−08 | Xq28-CGA high |
| DNAH3\|55567 | dynein, axonemal, heavy chain 3 | 2.36 | 5.81E−10 | Xq28-CGA high |
| DNAH7\|56171 | dynein, axonemal, heavy chain 7 | 2.16 | 2.09E−05 | Xq28-CGA high |
| DNAJC22\|79962 | DnaJ (Hsp40) homolog, subfamily C, member 22 | 2.03 | 3.99E−06 | Xq28-CGA high |
| DNALI1\|7802 | dynein, axonemal, light intermediate chain 1 | 8.88 | 1.18E−07 | Xq28-CGA high |
| DPY19L2P1\|554236 | DPY19L2 pseudogene 1 | 2.35 | 0.000128592 | Xq28-CGA high |
| DSCR10\|259234 | Down syndrome critical region gene 10 (non-pro [symbol] | 4.72 | 2.66E−16 | Xq28-CGA high |
| DSCR4\|10281 | Down syndrome critical region gene 4 | 109.57 | 9.21E−27 | Xq28-CGA high |
| DSCR8\|84677 | Down syndrome critical region gene 8 | 2470.84 | 5.96E−28 | Xq28-CGA high |
| E2F8\|79733 | E2F transcription factor 8 | 2.29 | 3.82E−09 | Xq28-CGA high |
| EFHC2\|80258 | EF-hand domain (C-terminal) containing 2 | 4.41 | 7.66E−06 | Xq28-CGA high |
| EGF\|1950 | epidermal growth factor | 3.74 | 1.76E−07 | Xq28-CGA high |
| ELAVL4\|1996 | ELAV like neuron-specific RNA binding protein 4 | 2.26 | 4.54E−06 | Xq28-CGA high |
| ELOVL2\|54898 | ELOVL fatty add elongase 2 | 3.7 | 3.24E−08 | Xq28-CGA high |
| ENPP4\|22875 | ectonucleotide pyrophosphatase/phosphodieste [symbol] | 3.06 | 4.19E−06 | Xq28-CGA high |
| ENPP5\|59084 | ectonucleotide pyrophosphatase/phosphodieste [symbol] | 4.98 | 1.50E−06 | Xq28-CGA high |
| EPHX4\|253152 | epoxide hydrolase 4 | 3.73 | 1.05E−04 | Xq28-CGA high |
| ERC2\|26059 | ELKS/RAB6-interacting/CAST family member 2 | 2.47 | 7.71E−07 | Xq28-CGA high |
| ERRFI1\|54206 | ERBB receptor feed back inhibitor 1 | 2.05 | 7.88E−08 | Xq28-CGA high |
| ESRRG\|2104 | estrogen-related receptor gamma | 4.89 | 1.36E−04 | Xq28-CGA high |
| EYA1\|2138 | eyes absent homolog 1 (Drosophila) | 40.69 | 6.57E−09 | Xq28-CGA high |
| F5\|2153 | coagulation factor V (proaccelerin, labile factor) | 2.39 | 3.01E−05 | Xq28-CGA high |
| FABP6\|2172 | fatty acid binding protein 6, ileal | 15.12 | 6.38E−07 | Xq28-CGA high |
| FABP7\|2173 | fatty acid binding protein 7, brain | 5.61 | 4.79E−05 | Xq28-CGA high |
| FAM106A\|80039 | family with sequence similarity 106, member A | 2.91 | 8.62E−05 | Xq28-CGA high |
| FAM106C\|100129396 | family with sequence similarity 106, member C, [symbol] | 2.62 | 4.24E−07 | Xq28-CGA high |
| FAM133A\|286499 | family with sequence similarity 133, member A | 85.36 | 1.01E−24 | Xq28-CGA high |
| FAM181B\|220382 | family with sequence similarity 181, member B | 2.58 | 5.11E−05 | Xq28-CGA high |
| FAM196B\|100131897 | family with sequence similarity 196, member B | 3.67 | 1.59E−05 | Xq28-CGA high |
| FAM46D\|169966 | family with sequence similarity 46, member D | 3.06 | 4.07E−05 | Xq28-CGA high |
| FAM81B\|153643 | family with sequence similarity 81, member B | 2.77 | 5.78E−05 | Xq28-CGA high |
| FBXL13\|222235 | F-box and leucine-rich repeat protein 13 | 2.47 | 2.05E−11 | Xq28-CGA high |
| FER1L4\|80307 | fer-1-like 4 (C. elegans), pseudogene | 2.43 | 7.52E−06 | Xq28-CGA high |
| FGF2\|2247 | fibroblast growth factor 2 (basic) | 3.96 | 1.07E−06 | Xq28-CGA high |
| FHAD1\|114827 | forkhead-associated (FHA) phosphopeptide bind [symbol] | 4.6 | 2.36E−09 | Xq28-CGA high |
| FIGF\|2277 | c-fos induced growth factor (vascular endothelia [symbol] | 2.69 | 2.20E−06 | Xq28-CGA high |
| FKBP1B\|2281 | FK506 binding protein 1B, 12.6 kDa | 2.35 | 1.77E−07 | Xq28-CGA high |
| FLI36000\|284124 | uncharacterized FLI36000 | 21.56 | 3.27E−13 | Xq28-CGA high |
| FLI44606\|401207 | | 2.53 | 1.53E−06 | Xq28-CGA high |
| FMNL2\|114793 | formin-like 2 | 2.09 | 1.37E−07 | Xq28-CGA high |
| FOXA3\|3171 | forkhead box A3 | 2.6 | 4.37E−05 | Xq28-CGA high |
| FOXR2\|139628 | forkhead box R2 | 2.42 | 1.05E−10 | Xq28-CGA high |
| FRAS1\|80144 | Fraser syndrome 1 | 3.4 | 8.26E−05 | Xq28-CGA high |
| G6PC2\|57818 | glucose-6-phosphatase, catalytic, 2 | 2.57 | 1.53E−04 | Xq28-CGA high |
| GABRA3\|2556 | gamma-aminobutyric acid (GABA) A receptor, al [symbol] | 530.61 | 4.59E−39 | Xq28-CGA high |
| GABRB1\|2560 | gamma-aminobutyric acid (GABA) A receptor, be [symbol] | 3.14 | 1.41E−07 | Xq28-CGA high |
| GABRG2\|2566 | gamma-aminobutyric acid (GABA) A receptor, ga [symbol] | 7.02 | 5.68E−19 | Xq28-CGA high |
| GABRP\|2568 | gamma-aminobutyric acid (GABA) A receptor, pi [symbol] | 3.95 | 3.91E−06 | Xq28-CGA high |
| GABRQ\|55879 | gamma-aminobutyric acid (GABA) A receptor, th [symbol] | 9.7 | 2.72E−17 | Xq28-CGA high |
| GAGE12D\|100132399 | G antigen 12D | 399.62 | 1.14E−17 | Xq28-CGA high |
| GAGE12J\|729396 | G antigen 12J | 45.61 | 1.67E−18 | Xq28-CGA high |
| GAGE1\|2543 | G antigen 1 | 45.46 | 1.61E−16 | Xq28-CGA high |
| GAGE2A\|729447 | G antigen 2A | 21.71 | 4.92E−13 | Xq28-CGA high |
| GAGE2D\|729408 | G antigen 2D | 209.41 | 7.37E−17 | Xq28-CGA high |
| GAGE4\|2576 | G antigen 4 | 300.81 | 1.27E−18 | Xq28-CGA high |
| GAGE8\|100101629 | G antigen 8 | 12.62 | 5.69E−13 | Xq28-CGA high |
| GALNT8\|26290 | polypeptide N-acetylgalactosaminyltransferase 8 [symbol] | 2.32 | 1.82E−08 | Xq28-CGA high |
| GAP43\|2596 | growth associated protein 43 | 4.67 | 1.31E−06 | Xq28-CGA high |
| GAS2\|2620 | growth arrest-specific 2 | 2.16 | 3.79E−06 | Xq28-CGA high |
| GBA3\|57733 | glucosidase, beta, acid 3 (gene/pseudogene) | 4.36 | 1.08E−06 | Xq28-CGA high |
| GDNF\|2668 | glial cell derived neurotrophic factor | 6.24 | 7.41E−07 | Xq28-CGA high |
| GJC3\|349149 | gap junction protein, gamma 3, 30.2 kDa | 2.03 | 2.74E−05 | Xq28-CGA high |
| GLB1L\|79411 | galactosidase, beta 1-like | 2.76 | 2.05E−07 | Xq28-CGA high |
| GLRB\|2743 | glycine receptor, beta | 6.29 | 4.68E−05 | Xq28-CGA high |
| GNGT1\|2792 | guanine nucleotide binding protein (G protein), g [symbol] | 22.02 | 2.12E−19 | Xq28-CGA high |
| GPR126\|57211 | | 3.45 | 5.41E−07 | Xq28-CGA high |
| GPR152\|390212 | G protein-coupled receptor 152 | 3.67 | 3.07E−05 | Xq28-CGA high |
| GPR158\|57512 | G protein-coupled receptor 158 | 7.06 | 2.44E−09 | Xq28-CGA high |
| GPR81\|27198 | hydroxycarboxylic acid receptor 1 | 4.5 | 9.46E−07 | Xq28-CGA high |
| GRB14\|2888 | growth factor receptor-bound protein 14 | 3.68 | 6.32E−05 | Xq28-CGA high |
| GRIA2\|2891 | glutamate receptor, ionotropic, AMPA 2 | 6.35 | 3.90E−10 | Xq28-CGA high |
| GSTT2\|2953 | glutathione S-transferase theta 2 | 3.36 | 3.47E−05 | Xq28-CGA high |
| GTSF1\|121355 | gametocyte specific factor 1 | 10.19 | 5.52E−13 | Xq28-CGA high |
| GUCY1B2\|2974 | guanylate cyclase 1, soluble, beta 2 (pseudogene [symbol] | 2.42 | 3.92E−06 | Xq28-CGA high |
| GULP1\|51454 | GULP, engulfment adaptor PTB domain containi [symbol] | 2.01 | 3.62E−05 | Xq28-CGA high |
| GYPE\|2996 | glycophorin E (MNS blood group) | 2.37 | 2.66E−05 | Xq28-CGA high |
| HAPLN1\|1404 | hyaluronan and proteoglycan link protein 1 | 2.44 | 9.80E−06 | Xq28-CGA high |
| HAPLN2\|60484 | hyaluronan and proteoglycan link protein 2 | 2.02 | 1.21E−04 | Xq28-CGA high |
| HBE1\|3046 | hemoglobin, epsilon 1 | 17.69 | 6.64E−11 | Xq28-CGA high |
| HBG2\|3048 | hemoglobin, gamma G | 3.68 | 1.18E−06 | Xq28-CGA high |
| HERC2P4\|440362 | hect domain and RLD 2 pseudogene 4 | 2.31 | 1.22E−09 | Xq28-CGA high |
| HFE2\|148738 | hemochromatosis type 2 (juvenile) | 2.72 | 5.88E−05 | Xq28-CGA high |
| HFM1\|164045 | HFM1, ATP-dependent DNA helicase homolog (S [symbol] | 2.53 | 3.95E−07 | Xq28-CGA high |
| HHATL\|57467 | hedgehog acyltransferase-like | 5.41 | 8.80E−06 | Xq28-CGA high |
| HHLA2\|11148 | HERV-H LTR-associating 2 | 3.23 | 3.53E−06 | Xq28-CGA high |
| HIST1H2BF\|8343 | histone cluster 1, H2bf | 2.45 | 3.66E−06 | Xq28-CGA high |
| HIST2H4A\|8370 | histone cluster 2, H4a | 2.03 | 1.36E−05 | Xq28-CGA high |
| HIVEP3\|59269 | human immunodeficiency virus type I enhancer | 2.08 | 3.98E−08 | Xq28-CGA high |
| HMGA2\|8091 | high mobility group AT-hook 2 | 5.05 | 1.37E−08 | Xq28-CGA high |
| HOOK1\|51361 | hook microtubule-tethering protein 1 | 2.89 | 5.09E−06 | Xq28-CGA high |
| HORMAD1\|84072 | HORMA domain containing 1 | 26.19 | 2.43E−06 | Xq28-CGA high |
| HOXA1\|3198 | homeobox A1 | 2.06 | 4.88E−05 | Xq28-CGA high |
| HOXA2\|3199 | homeobox A2 | 2.1 | 0.000153759 | Xq28-CGA high |
| HOXA3\|3200 | homeobox A3 | 2.87 | 7.97E−05 | Xq28-CGA high |
| HOXC10\|3226 | homeobox C10 | 2.47 | 2.92E−06 | Xq28-CGA high |
| HOXD10\|3236 | homeobox D10 | 2.1 | 4.39E−06 | Xq28-CGA high |
| HOXD11\|3237 | homeobox D11 | 2.87 | 1.30E−05 | Xq28-CGA high |
| HOXD13\|3239 | homeobox D13 | 6.95 | 1.38E−11 | Xq28-CGA high |
| HS3ST5\|222537 | heparan sulfate (glucosamine) 3-O-sulfotransfer [symbol] | 3.04 | 8.25E−06 | Xq28-CGA high |
| HSD17B3\|3293 | hydroxysteroid (17-beta) dehydrogenase 3 | 2.34 | 1.58E−05 | Xq28-CGA high |
| HSPC072\|29075 | long intergenic non-protein coding RNA 652 | 3.03 | 6.30E−09 | Xq28-CGA high |
| HTN1\|3346 | histatin 1 | 27.42 | 4.20E−05 | Xq28-CGA high |
| HTR2C\|3358 | 5-hydroxytryptamme (serotonin) receptor 2C, G | 3.58 | 9.07E−14 | Xq28-CGA high |
| HTR3E\|285242 | 5-hydroxytryptamine (serotonin) receptor 3E, io [symbol] | 2.43 | 3.95E−08 | Xq28-CGA high |
| HULC\|728655 | hepatocellular carcinoma up-regulated long non [symbol] | 28.25 | 1.12E−08 | Xq28-CGA high |
| HYDIN\|54768 | HYDIN, axonemal central pair apparatus protein | 2.82 | 3.89E−05 | Xq28-CGA high |
| ID4\|3400 | inhibitor of DNA binding 4, dominant negative h [symbol] | 2.5 | 7.97E−05 | Xq28-CGA high |
| IGF2BP3\|10643 | insulin-like growth factor 2 mRNA binding protei [symbol] | 2.58 | 4.48E−06 | Xq28-CGA high |
| IGFN1\|91156 | immunoglobulin-like and fibronectin type III don [symbol] | 2.41 | 1.67E−06 | Xq28-CGA high |
| IL13RA2\|3598 | interleukin 13 receptor, alpha 2 | 41.62 | 3.72E−14 | Xq28-CGA high |
| IL1RAPL1\|11141 | interleukin 1 receptor accessory protein-like 1 | 5.55 | 3.11E−05 | Xq28-CGA high |
| IL31RA\|133396 | interleukin 31 receptor A | 2.54 | 0.000141763 | Xq28-CGA high |
| INADL\|10207 | InaD-like (Drosophila) | 2.11 | 1.36E−04 | Xq28-CGA high |
| INTU\|27152 | inturned planar cell polarity protein | 2.06 | 1.94E−08 | Xq28-CGA high |
| IQUB\|154865 | IQ motif and ubiquitin domain containing | 2.22 | 1.03E−07 | Xq28-CGA high |
| ISL2\|64843 | ISL LIM homeobox 2 | 4.66 | 1.46E−08 | Xq28-CGA high |
| ITGB3\|3690 | integrin, beta 3 (platelet glycoprotein IIIa, antige [symbol] | 2.42 | 1.98E−05 | Xq28-CGA high |
| ITPRIPL1\|150771 | inositol 1,4,5-trisphosphate receptor interacting | 2.42 | 1.44E−05 | Xq28-CGA high |
| KC6\|641516 | keratoconus gene 6 | 2.67 | 4.58E−11 | Xq28-CGA high |
| KCNA6\|3742 | potassium voltage-gated channel, shaker-relate [symbol] | 2.36 | 6.63E−05 | Xq28-CGA high |
| KCNAB3\|9196 | potassium voltage-gated channel, shaker-relate [symbol] | 2.09 | 1.68E−05 | Xq28-CGA high |
| KCNC2\|3747 | potassium voltage-gated channel, Shaw-related [symbol] | 2.09 | 2.21E−08 | Xq28-CGA high |
| KCNH5\|27133 | potassium voltage-gated channel, subfamily H (e [symbol] | 2.6 | 5.13E−12 | Xq28-CGA high |
| KCNJ10\|3766 | potassium inwardly-rectifying channel, subfamil [symbol] | 3.49 | 6.79E−08 | Xq28-CGA high |
| KCNMB2\|10242 | potassium large conductance calcium-activated [symbol] | 2.34 | 1.16E−08 | Xq28-CGA high |
| KCNS1\|3787 | potassium voltage-gated channel, delayed-rectif [symbol] | 2.67 | 4.00E−05 | Xq28-CGA high |
| KIAA0664P3\|1001323 | clustered mitochondria (cluA/CLU1) homolog ps [symbol] | 2.76 | 6.68E−05 | Xq28-CGA high |
| KIAA1324L\|222223 | KIAA1324-like | 2.71 | 1.38E−07 | Xq28-CGA high |
| KIAA1377\|57562 | | 2.39 | 2.61E−07 | Xq28-CGA high |
| KIAA1383\|54627 | microtubule-associated protein 10 | 2.54 | 7.63E−08 | Xq28-CGA high |
| KIF17\|57576 | kinesin family member 17 | 2.73 | 1.27E−04 | Xq28-CGA high |
| KIF5A\|3798 | kinesin family member 5A | 3.68 | 1.28E−09 | Xq28-CGA high |
| KLF14\|136259 | Kruppel-like factor 14 | 4.57 | 1.86E−11 | Xq28-CGA high |
| KLF17\|128209 | Kruppel-like factor 17 | 2.17 | 2.09E−05 | Xq28-CGA high |
| KLHL13\|90293 | kelch-like family member 13 | 4.48 | 5.29E−05 | Xq28-CGA high |
| KLHL23\|151230 | kelch-fike family member 23 | 2.44 | 4.64E−07 | Xq28-CGA high |
| KLK2\|3817 | kallikrein-related peptidase 2 | 6.77 | 2.54E−08 | Xq28-CGA high |
| KRT18\|3875 | keratin 18 | 5.23 | 5.86E−08 | Xq28-CGA high |
| LCTL\|197021 | lactase-like | 2.59 | 3.49E−05 | Xq28-CGA high |
| LGALS12\|85329 | lectin, galactoside-binding, soluble, 12 | 3.39 | 3.69E−08 | Xq28-CGA high |
| LIN28A\|79727 | lin-28 homolog A (C. elegans) | 2.57 | 5.88E−07 | Xq28-CGA high |
| LIN28B\|389421 | lin-28 homolog B (C. elegans) | 5.07 | 1.09E−08 | Xq28-CGA high |
| LMOD2\|442721 | leiomodin 2 (cardiac) | 2.2 | 3.12E−07 | Xq28-CGA high |
| LOC100128675\|100128675 | | 15.63 | 6.73E−07 | Xq28-CGA high |
| LOC100130386\|100130386 | | 2.69 | 5.98E−06 | Xq28-CGA high |
| LOC100133469\|100133469 | | 1402.15 | 4.13E−30 | Xq28-CGA high |
| LOC100190938\|100190938 | | 3.14 | 3.54E−08 | Xq28-CGA high |
| LOC100240726\|100240726 | | 2.76 | 5.77E−05 | Xq28-CGA high |
| LOC100271722\|100271722 | | 2.04 | 1.35E−05 | Xq28-CGA high |
| LOC116437\|116437 | | 2.23 | 2.01E−05 | Xq28-CGA high |
| LOC134466\|134466 | | 5.45 | 2.54E−09 | Xq28-CGA high |
| LOC146481\|146481 | | 8.29 | 1.42E−04 | Xq28-CGA high |
| LOC148824\|148824 | | 2.32 | 2.45E−05 | Xq28-CGA high |
| LOC150197\|150197 | | 2.73 | 4.51E−06 | Xq28-CGA high |
| LOC153910\|153910 | | 5.04 | 1.12E−10 | Xq28-CGA high |
| LOC162632\|162632 | | 2.36 | 7.79E−05 | Xq28-CGA high |
| LOC220594\|220594 | | 3.17 | 6.92E−05 | Xq28-CGA high |
| LOC220930\|220930 | | 2.19 | 0.000106118 | Xq28-CGA high |
| LOC257358\|257358 | | 2.12 | 9.99E−07 | Xq28-CGA high |
| LOC285419\|285419 | | 2.17 | 9.57E−05 | Xq28-CGA high |
| LOC285501\|285501 | | 4.41 | 5.54E−17 | Xq28-CGA high |
| LOC285548\|285548 | | 2.74 | 9.57E−07 | Xq28-CGA high |
| LOC286002\|286002 | | 5.97 | 2.13E−07 | Xq28-CGA high |
| LOC286467\|286467 | | 3.3 | 3.82E−09 | Xq28-CGA high |
| LOC339535\|339535 | | 3.3 | 4.53E−06 | Xq28-CGA high |
| LOC374491\|374491 | | 2.68 | 6.92E−08 | Xq28-CGA high |
| LOC399815\|399815 | chromosome 10 open reading frame 88 pseudog [symbol] | 2.04 | 1.51E−05 | Xq28-CGA high |
| LOC399959\|399959 | | 4.61 | 8.73E−07 | Xq28-CGA high |
| LOC440563\|440563 | | 2.05 | 1.08E−05 | Xq28-CGA high |
| LOC440905\|440905 | | 2.84 | 5.41E−07 | Xq28-CGA high |
| LOC441046\|441046 | | 3.08 | 3.17E−08 | Xq28-CGA high |
| LOC441294\|441294 | | 4.98 | 1.05E−08 | Xq28-CGA high |
| LOC441601\|441601 | septin 7 pseudogene | 3.35 | 3.65E−12 | Xq28-CGA high |
| LOC645323\|645323 | | 2.92 | 7.58E−07 | Xq28-CGA high |
| LOC646627\|646627 | | 8.95 | 6.72E−07 | Xq28-CGA high |
| LOC648691\|648691 | | 2.64 | 4.38E−09 | Xq28-CGA high |
| LOC728819\|728819 | | 6.45 | 1.05E−13 | Xq28-CGA high |
| LOC84740\|84740 | | 2.05 | 1.49E−07 | Xq28-CGA high |
| LPAR3\|23566 | lysophosphatidic acid receptor 3 | 3.19 | 8.77E−05 | Xq28-CGA high |
| LPPR1\|54886 | lipid phosphate phosphatase-related protein typ [symbol] | 4.08 | 2.89E−08 | Xq28-CGA high |
| LPPR5\|163404 | lipid phosphate phosphatase-related protein typ [symbol] | 2.13 | 5.11E−05 | Xq28-CGA high |
| LRCH2\|57631 | leucine-rich repeats and calponin homology (CH) [symbol] | 6.46 | 9.89E−09 | Xq28-CGA high |
| LRP4\|4038 | low density lipoprotein receptor-related protein | 2.53 | 4.73E−08 | Xq28-CGA high |
| LRRC52\|440699 | leucine rich repeat containing 52 | 3.49 | 3.81E−05 | Xq28-CGA high |
| LRRC69\|100130742 | leucine rich repeat containing 69 | 3.12 | 9.90E−11 | Xq28-CGA high |
| LRRIQ1\|84125 | leucine-rich repeats and IQ motif containing 1 | 7.44 | 3.41E−07 | Xq28-CGA high |
| LYG2\|254773 | lysozyme G-like 2 | 4.85 | 3.08E−07 | Xq28-CGA high |
| MAEL\|84944 | maelstrom spermatogenic transposon silencer | 3.41 | 2.44E−05 | Xq28-CGA high |
| MAGEA10\|4109 | melanoma antigen family A, 10 | 1422.65 | 7.86E−33 | Xq28-CGA high |
| MAGEA11\|4110 | melanoma antigen family A, 11 | 93.76 | 3.72E−22 | Xq28-CGA high |
| MAGEA12\|4111 | melanoma antigen family A, 12 | 1533.86 | 3.82E−40 | Xq28-CGA high |
| MAGEA1\|4100 | melanoma antigen family A, 1 (directs expressio [symbol] | 1309.3 | 3.67E−32 | Xq28-CGA high |
| MAGEA2\|4101 | melanoma antigen family A, 2 | 2276.53 | 1.98E−40 | Xq28-CGA high |
| MAGEA3\|4102 | melanoma antigen family A, 3 | 2434.59 | 3.72E−40 | Xq28-CGA high |
| MAGEA4\|4103 | melanoma antigen family A, 4 | 365.89 | 9.45E−18 | Xq28-CGA high |
| MAGEA5\|4104 | melanoma antigen family A, 5 | 10.12 | 3.15E−25 | Xq28-CGA high |
| MAGEA6\|4105 | melanoma antigen family A, 6 | 2536.59 | 3.81E−40 | Xq28-CGA high |
| MAGEA8\|4107 | melanoma antigen family A, 8 | 2.32 | 2.02E−05 | Xq28-CGA high |
| MAGEA9B\|728269 | melanoma antigen family A, 9B | 11.92 | 2.73E−14 | Xq28-CGA high |
| MAGEB16\|139604 | melanoma antigen family B, 16 | 2.6 | 2.58E−19 | Xq28-CGA high |
| MAGEB1\|4112 | melanoma antigen family B, 1 | 4.62 | 1.91E−17 | Xq28-CGA high |
| MAGEB2\|4113 | melanoma antigen family B, 2 | 47.51 | 1.38E−19 | Xq28-CGA high |
| MAGEB6\|158809 | melanoma antigen family B, 6 | 8.62 | 4.45E−18 | Xq28-CGA high |
| MAGEC1\|9947 | melanoma antigen family C, 1 | 737.15 | 2.15E−30 | Xq28-CGA high |
| MAGEC2\|51438 | melanoma antigen family C, 2 | 2411.41 | 7.48E−26 | Xq28-CGA high |
| MAGEC3\|139081 | melanoma antigen family C, 3 | 3.64 | 2.47E−12 | Xq28-CGA high |
| MAOA\|4128 | monoamine oxidase A | 2.94 | 1.23E−05 | Xq28-CGA high |
| MAP9\|79884 | microtubule-associated protein 9 | 4.12 | 2.29E−06 | Xq28-CGA high |
| MAT1A\|4143 | methionine adenosyltransferase I, alpha | 5.07 | 2.73E−06 | Xq28-CGA high |
| MEGF10\|84466 | multiple EGF-like-domains 10 | 18.91 | 1.58E−09 | Xq28-CGA high |
| MGAT4C\|25834 | mannosyl (alpha-1,3-)-glycoprotein beta-1,4-N-a [symbol] | 7.44 | 2.17E−14 | Xq28-CGA high |
| MGC16121\|84848 | | 2.03 | 5.68E−05 | Xq28-CGA high |
| MMEL1\|79258 | membrane metallo-endopeptidase-like 1 | 3.01 | 4.77E−05 | Xq28-CGA high |
| MMP16\|4325 | matrix metallopeptidase 16 (membrane-inserte [symbol] | 4.58 | 1.21E−05 | Xq28-CGA high |
| MOG\|4340 | myelin oligodendrocyte glycoprotein | 4.42 | 1.62E−05 | Xq28-CGA high |
| MORC1\|27136 | MORC famiy CW-type zinc finger 1 | 9.55 | 1.50E−10 | Xq28-CGA high |
| MPPED2\|744 | metallophosphoesterase domain containing 2 | 2.21 | 1.16E−04 | Xq28-CGA high |
| MRGPRX3\|117195 | MAS-related GPR, member X3 | 11.36 | 1.44E−10 | Xq28-CGA high |
| MS4A15\|219995 | membrane-spanning 4-domains, subfamily A, m [symbol] | 4.4 | 1.45E−04 | Xq28-CGA high |
| MST4\|51765 | | 3.18 | 5.54E−07 | Xq28-CGA high |
| MT1G\|4495 | metallothionein 1G | 2.32 | 2.01E−05 | Xq28-CGA high |
| MT1H\|4496 | metallothionein 1H | 2.55 | 4.65E−05 | Xq28-CGA high |
| MUC15\|143662 | mucin 15, cell surface associated | 17.34 | 1.07E−11 | Xq28-CGA high |
| MYADML2\|255275 | myeloid-associated differentiation marker-like 2 [symbol] | 2.07 | 5.51E−07 | Xq28-CGA high |
| MYBPC1\|4604 | myosin binding protein C, slow type | 3.42 | 5.56E−07 | Xq28-CGA high |
| MYH13\|8735 | myosin, heavy chain 13, skeletal muscle | 4.59 | 2.26E−08 | Xq28-CGA high |
| MYH15\|22989 | myosin, heavy chain 15 | 2.13 | 1.90E−06 | Xq28-CGA high |
| MYOT\|9499 | myotillin | 2.45 | 6.28E−06 | Xq28-CGA high |
| MYOZ3\|91977 | myozenin 3 | 2.49 | 4.58E−05 | Xq28-CGA high |
| NAA11\|84779 | N(alpha)-acetyltransferase 11, NatA catalytic su [symbol] | 8.03 | 2.57E−18 | Xq28-CGA high |
| NBPF22P\|285622 | neuroblastoma breakpoint family, member 22, p [symbol] | 4.32 | 3.64E−08 | Xq28-CGA high |
| NBPF4\|148545 | neuroblastoma breakpoint family, member 4 | 10.14 | 8.18E−09 | Xq28-CGA high |
| NBPF6\|653149 | neuroblastoma breakpoint family, member 6 | 13.18 | 2.09E−07 | Xq28-CGA high |
| NCRNA00189\|193629 | long intergenic non-protein coding RNA 189 | 16.16 | 5.66E−07 | Xq28-CGA high |
| NEB\|4703 | nebulin | 2.34 | 3.07E−09 | Xq28-CGA high |
| NECAB1\|64168 | N-terminal EF-hand calcium binding protein 1 | 2.02 | 1.13E−09 | Xq28-CGA high |
| NFE2L3\|9603 | nuclear factor, erythroid 2-like 3 | 2.68 | 2.30E−09 | Xq28-CGA high |
| NFIA\|4774 | nuclear factor I/A | 2.24 | 9.57E−09 | Xq28-CGA high |
| NLGN3\|54413 | neuroligin 3 | 2.67 | 4.69E−06 | Xq28-CGA high |
| NLRP11\|204801 | NLR family, pyrin domain containing 11 | 5.18 | 5.32E−12 | Xq28-CGA high |
| NLRP4\|147945 | NLR family, pyrin domain containing 4 | 4.25 | 3.07E−09 | Xq28-CGA high |
| NOX4\|50507 | NADPH oxidase 4 | 3.66 | 2.75E−07 | Xq28-CGA high |
| NPY6R\|4888 | neuropeptide Y receptor Y6 (pseudogene) | 3.12 | 4.69E−09 | Xq28-CGA high |
| NXPH1\|30010 | neurexophilin 1 | 3 | 3.58E−06 | Xq28-CGA high |
| OCIAD2\|132299 | OCIA domain containing 2 | 2.48 | 9.31E−05 | Xq28-CGA high |
| OCLM\|10896 | oculomedin | 2.03 | 1.08E−09 | Xq28-CGA high |
| ODZ1\|10178 | teneurin transmembrane protein 1 | 2.23 | 1.64E−06 | Xq28-CGA high |
| OLFM3\|118427 | olfactomedin 3 | 2.32 | 9.03E−07 | Xq28-CGA high |
| OLIG2\|10215 | oligodendrocyte lineage transcription factor 2 | 25.54 | 3.80E−06 | Xq28-CGA high |
| OMP\|4975 | olfactory marker protein | 2.38 | 1.18E−08 | Xq28-CGA high |
| OR1J2\|26740 | olfactory receptor, family 1, subfamily J, membe [symbol] | 2.01 | 6.40E−06 | Xq28-CGA high |
| OR2A1\|346528 | olfactory receptor, family 2, subfamily A, membe [symbol] | 3.14 | 2.58E−06 | Xq28-CGA high |
| OR2A25\|392138 | olfactory receptor, family 2, subfamily A, membe [symbol] | 5.45 | 9.70E−07 | Xq28-CGA high |
| OR2A4\|79541 | olfactory receptor, family 2, subfamily A, membe [symbol] | 3.39 | 4.54E−06 | Xq28-CGA high |
| OR2A7\|401427 | olfactory receptor, family 2, subfamily A, membe [symbol] | 2.49 | 2.32E−05 | Xq28-CGA high |
| OR2A9P\|441295 | olfactory receptor, family 2, subfamily A, membe [symbol] | 3.33 | 1.98E−07 | Xq28-CGA high |
| OR2B6\|26212 | olfactory receptor, family 2, subfamily B, membe [symbol] | 2.16 | 1.03E−04 | Xq28-CGA high |
| OR2H2\|7932 | olfactory receptor, family 2, subfamily H, membe [symbol] | 6.93 | 5.96E−06 | Xq28-CGA high |
| OR51B5\|282763 | olfactory receptor, family 51, subfamily B, memb [symbol] | 4.35 | 3.04E−11 | Xq28-CGA high |
| OR56B4\|196335 | olfactory receptor, family 56, subfamily B, memb [symbol] | 4.94 | 7.66E−06 | Xq28-CGA high |
| OR7E5P\|219445 | olfactory receptor, family 7, subfamily E, membe [symbol] | 2.38 | 2.35E−12 | Xq28-CGA high |
| OR8A1\|390275 | olfactory receptor, family 8, subfamily A, membe [symbol] | 4.39 | 1.09E−20 | Xq28-CGA high |
| OXGR1\|27199 | oxoglutarate (alpha-ketoglutarate) receptor 1 | 3.49 | 0.000140551 | Xq28-CGA high |
| PAGE1\|8712 | P antigen family, member 1 (prostate associated [symbol] | 6.35 | 8.46E−11 | Xq28-CGA high |
| PAGE2B\|389860 | P antigen family, member 2B | 19.39 | 1.50E−12 | Xq28-CGA high |
| PAGE5\|90737 | P antigen family, member 5 (prostate associated [symbol] | 2367.74 | 5.52E−23 | Xq28-CGA high |
| PAH\|5053 | phenylalanine hydroxylase | 3.81 | 3.54E−06 | Xq28-CGA high |
| PALM3\|342979 | paralemmin 3 | 2.67 | 2.20E−05 | Xq28-CGA high |
| PANX3\|116337 | pannexin 3 | 3.87 | 4.49E−19 | Xq28-CGA high |
| PAR5\|8123 | Prader Willi/Angelman region RNA 5 | 2.13 | 1.30E−05 | Xq28-CGA high |
| PASD1\|139135 | PAS domain containing 1 | 2.29 | 5.56E−15 | Xq28-CGA high |
| PCDH7\|5099 | protocadherin 7 | 2.25 | 3.74E−05 | Xq28-CGA high |
| PCDHB18\|54660 | | 2.09 | 1.13E−04 | Xq28-CGA high |
| PCDHGA2\|56113 | protocadherin gamma subfamily A, 2 | 2.48 | 1.02E−05 | Xq28-CGA high |
| PCSK1\|5122 | proprotein convertase subtilisin/kexin type 1 | 3.29 | 3.12E−05 | Xq28-CGA high |
| PDC\|5132 | phosducin | 3.81 | 1.02E−06 | Xq28-CGA high |
| PDIA2\|64714 | protein disulfide isomerase family A, member 2 | 3.74 | 3.41E−07 | Xq28-CGA high |
| PDK4\|5166 | pyruvate dehydrogenase kinase, isozyme 4 | 2.6 | 2.60E−05 | Xq28-CGA high |
| PEG10\|23089 | paternally expressed 10 | 2.16 | 9.13E−06 | Xq28-CGA high |
| PEX5L\|51555 | peroxisomal biogenesis factor 5-like | 2.32 | 5.38E−07 | Xq28-CGA high |
| PGAM2\|5224 | phosphoglycerate mutase 2 (muscle) | 2.01 | 1.81E−08 | Xq28-CGA high |
| PI15\|51050 | peptidase inhibitor 15 | 5.2 | 1.20E−06 | Xq28-CGA high |
| PKIA\|5569 | protein kinase (cAMP-dependent, catalytic) inhi [symbol] | 3.07 | 0.00012646 | Xq28-CGA high |
| PLAC1\|10761 | placenta-specific 1 | 3.58 | 1.67E−09 | Xq28-CGA high |
| PLCB1\|23236 | phospholipase C, beta 1 (phosphoinositide-speci [symbol] | 8.55 | 1.87E−19 | Xq28-CGA high |
| PLCB4\|5332 | phospholipase C, beta 4 | 12.5 | 4.71E−07 | Xq28-CGA high |
| PLCE1\|51196 | phospholipase C, epsilon 1 | 2.3 | 6.81E−05 | Xq28-CGA high |
| PLEKHB1\|58473 | pleckstrin homology domain containing, family B [symbol] | 3.79 | 3.13E−07 | Xq28-CGA high |
| PLS1\|5357 | plastin 1 | 2.64 | 6.71E−06 | Xq28-CGA high |
| PMFBP1\|83449 | polyamine modulated factor 1 binding protein 1 | 2.59 | 6.38E−08 | Xq28-CGA high |
| POU5F1B\|5462 | POU class 5 homeobox 1B | 3.69 | 1.48E−09 | Xq28-CGA high |
| POU6F2\|11281 | POU class 6 homeobox 2 | 2.55 | 1.28E−12 | Xq28-CGA high |
| PPIEL\|728448 | peptidylprolyl isomerase E-like pseudogene | 2.09 | 3.83E−05 | Xq28-CGA high |
| PPP1R1B\|84152 | protein phosphatase 1, regulatory (inhibitor) sub [symbol] | 2.91 | 5.01E−05 | Xq28-CGA high |
| PPP1R1C\|151242 | protein phosphatase 1, regulatory (inhibitor) sub [symbol] | 5.08 | 1.17E−10 | Xq28-CGA high |
| PPP1R9A\|55607 | protein phosphatase 1, regulatory subunit 9A | 2.39 | 9.20E−07 | Xq28-CGA high |
| PPP2R2B\|5521 | protein phosphatase 2, regulatory subunit B, bet [symbol] | 2.35 | 9.31E−05 | Xq28-CGA high |
| PRKAA2\|5563 | protein kinase, AMP-activated, alpha 2 catalytic [symbol] | 3.72 | 1.16E−05 | Xq28-CGA high |
| PRSS21\|10942 | protease, serine, 21 (testisin) | 4.9 | 5.77E−05 | Xq28-CGA high |
| PSG9\|5678 | pregnancy specific beta-1-glycoprotein 9 | 2.87 | 3.35E−05 | Xq28-CGA high |
| PTN\|5764 | pleiotrophin | 2.17 | 2.20E−05 | Xq28-CGA high |
| PTPN20B\|26095 | | 5.03 | 1.97E−05 | Xq28-CGA high |
| PYY2\|23615 | peptide YY, 2 (pseudogene) | 2.06 | 1.36E−04 | Xq28-CGA high |
| RAB26\|25837 | RAB26, member RAS oncogene family | 2.73 | 3.05E−09 | Xq28-CGA high |
| RAB3IP\|117177 | RAB3A interacting protein | 2.68 | 5.23E−07 | Xq28-CGA high |
| RAPGEF4\|11069 | Rap guanine nucleotide exchange factor (GEF) 4 | 2.89 | 2.90E−05 | Xq28-CGA high |
| RARB\|5915 | retinoic acid receptor, beta | 4.72 | 1.52E−06 | Xq28-CGA high |
| RBM20\|282996 | RNA binding motif protein 20 | 6.07 | 4.51E−07 | Xq28-CGA high |
| RBMS3\|27303 | RNA binding motif, single stranded interacting p [symbol] | 2.59 | 2.84E−05 | Xq28-CGA high |
| RCOR2\|283248 | REST corepressor 2 | 2.96 | 5.46E−05 | Xq28-CGA high |
| RFPL4B\|442247 | ret finger protein-like 4B | 2.53 | 5.32E−06 | Xq28-CGA high |
| RFX4\|5992 | regulatory factor X, 4 (influences HLA class II exp [symbol] | 2.57 | 4.43E−10 | Xq28-CGA high |
| RGNEF\|64283 | Rho guanine nucleotide exchange factor (GEF) 2 [symbol] | 2.23 | 3.17E−06 | Xq28-CGA high |
| RLBP1\|6017 | retinaldehyde binding protein 1 | 7.09 | 8.78E−09 | Xq28-CGA high |
| RNASE10\|338879 | ribonuclease, RNase A family, 10 (non-active) | 2.01 | 0.000118574 | Xq28-CGA high |
| RND2\|8153 | Rho family GTPase 2 | 3.68 | 1.26E−08 | Xq28-CGA high |
| RNF175\|285533 | ring finger protein 175 | 10.89 | 8.92E−10 | Xq28-CGA high |
| RPS15AP10\|728963 | ribosomal protein S15a pseudogene 10 | 2.07 | 1.12E−07 | Xq28-CGA high |
| RYR2\|6262 | ryanodine receptor 2 (cardiac) | 3.28 | 2.76E−06 | Xq28-CGA high |
| SCAND3\|114821 | | 2.01 | 5.38E−05 | Xq28-CGA high |
| SCARNA16\|677781 | small Cajal body-spedfic RNA 16 | 2.43 | 7.17E−07 | Xq28-CGA high |
| SCN2A\|6326 | sodium channel, voltage-gated, type II, alpha sub [symbol] | 2.19 | 1.44E−06 | Xq28-CGA high |
| SCN9A\|6335 | sodium channel, voltage-gated, type IX, alpha su [symbol] | 4.32 | 2.23E−06 | Xq28-CGA high |
| SCRG1\|11341 | stimulator of chondrogenesis 1 | 5.17 | 1.12E−04 | Xq28-CGA high |
| SCRN1\|9805 | secemin 1 | 4.15 | 6.09E−07 | Xq28-CGA high |
| SEMA3E\|9723 | sema domain, immunoglobulin domain (Ig), sho [symbol] | 9.39 | 5.40E−09 | Xq28-CGA high |
| SERPINA5\|5104 | serpin peptidase inhibitor, clade A (alpha-1 antip [symbol] | 6.08 | 1.42E−07 | Xq28-CGA high |
| SH2D6\|284948 | SH2 domain containing 6 | 2.21 | 3.12E−06 | Xq28-CGA high |
| SHROOM3\|57619 | shroom family member 3 | 4.64 | 1.46E−08 | Xq28-CGA high |
| SKAP1\|8631 | src kinase associated phosphoprotein 1 | 3.16 | 7.20E−05 | Xq28-CGA high |
| SLC10A4\|201780 | solute carrier family 10, member 4 | 2.53 | 1.80E−09 | Xq28-CGA high |
| SLC15A2\|6565 | solute carrier family 15 (oligopeptide transporte [symbol] | 2.31 | 1.64E−08 | Xq28-CGA high |
| SLC18A1\|6570 | solute carrier family 18 (vesicular monoamine tr [symbol] | 2.12 | 7.98E−05 | Xq28-CGA high |
| SLC22A13\|9390 | solute carrier family 22 (organic anion/urate trar [symbol] | 2.42 | 7.74E−05 | Xq28-CGA high |
| SLC23A3\|151295 | solute carrier family 23, member 3 | 2.14 | 9.02E−07 | Xq28-CGA high |
| SLC26A4\|5172 | solute carrier family 26 (anion exchanger), mem [symbol] | 3.23 | 4.85E−07 | Xq28-CGA high |
| SLC26A7\|115111 | solute carrier family 26 (anion exchanger), mem [symbol] | 2.43 | 3.31E−05 | Xq28-CGA high |
| SLC30A8\|160026 | solute carrier family 30 (zinc transporter), memb [symbol] | 5.18 | 1.88E−13 | Xq28-CGA high |
| SLC44A5\|204962 | solute carrier family 44, member 5 | 4.46 | 1.06E−08 | Xq28-CGA high |
| SLC5A12\|159963 | solute carrier family 5 (sodium/monocarboxylat [symbol] | 4.14 | 3.14E−12 | Xq28-CGA high |
| SLC6A13\|6540 | solute carrier family 6 (neurotransmitter transpo [symbol] | 2.08 | 2.53E−05 | Xq28-CGA high |
| SLC9A11\|284525 | solute carrier family 9, member C2 (putative) | 3.07 | 5.28E−08 | Xq28-CGA high |
| SLCO1A2\|6579 | solute carrier organic anion transporter family, n [symbol] | 18.61 | 2.14E−18 | Xq28-CGA high |
| SMC1B\|27127 | structural maintenance of chromosomes 1B | 2.52 | 3.15E−06 | Xq28-CGA high |
| SMEK3P\|139420 | SMEK homolog 3, suppressor of mek1 (Dictyoste [symbol] | 3.05 | 3.36E−17 | Xq28-CGA high |
| SMYD1\|150572 | SET and MYND domain containing 1 | 3.11 | 7.47E−08 | Xq28-CGA high |
| SOHLH1\|402381 | spermatogenesis and oogenesis specific basic he [symbol] | 3.48 | 8.79E−07 | Xq28-CGA high |
| SORL1\|6653 | sortilin-related receptor, L(DLR class) A repeats [symbol] | 2.27 | 2.39E−05 | Xq28-CGA high |
| SOSTDC1\|25928 | sclerostin domain containing 1 | 5.8 | 4.30E−05 | Xq28-CGA high |
| SP140L\|93349 | SP140 nuclear body protein-like | 2.23 | 1.21E−10 | Xq28-CGA high |
| SPAG17\|200162 | sperm associated antigen 17 | 2.04 | 4.74E−06 | Xq28-CGA high |
| SPAG4\|6676 | sperm associated antigen 4 | 2.37 | 2.61E−07 | Xq28-CGA high |
| SPATA17\|128153 | spermatogenesis associated 17 | 2.17 | 4.63E−06 | Xq28-CGA high |
| SPERT\|220082 | spermatid associated | 3.05 | 6.01E−06 | Xq28-CGA high |
| SPP1\|6696 | secreted phosphoprotein 1 | 3.57 | 1.36E−07 | Xq28-CGA high |
| SPRY4\|81848 | sprouty homolog 4 (Drosophila) | 2.46 | 4.90E−07 | Xq28-CGA high |
| SSX1\|6756 | synovial sarcoma, X breakpoint 1 | 11.7 | 1.86E−06 | Xq28-CGA high |
| SSX2\|6757 | synovial sarcoma, X breakpoint 2 | 28.85 | 6.07E−22 | Xq28-CGA high |
| SSX5\|6758 | synovial sarcoma, X breakpoint 5 | 7.49 | 3.80E−06 | Xq28-CGA high |
| SSX6\|280657 | synovial sarcoma, X breakpoint 6 (pseudogene) | 5.71 | 1.61E−06 | Xq28-CGA high |
| ST6GALNAC5\|81849 | ST6 (alpha-N-acetyl-neuraminyl-2,3-beta-galact [symbol] | 4.3 | 9.70E−07 | Xq28-CGA high |
| STARD4\|134429 | StAR-related lipid transfer (START) domain conta [symbol] | 2.15 | 4.31E−05 | Xq28-CGA high |
| STK31\|56164 | serine/threonine kinase 31 | 7.76 | 2.18E−11 | Xq28-CGA high |
| STK33\|65975 | serine/threonine kinase 33 | 2.69 | 4.34E−05 | Xq28-CGA high |
| SUMO4\|387082 | small ubiquitin-like modifier 4 | 2.18 | 2.64E−07 | Xq28-CGA high |
| SV2A\|9900 | synaptic vesicle glycoprotein 2A | 6.7 | 2.15E−12 | Xq28-CGA high |
| SYT1\|6857 | synaptotagmin I | 3.35 | 1.97E−08 | Xq28-CGA high |
| SYTL5\|94122 | synaptotagmin-like 5 | 5.5 | 9.53E−07 | Xq28-CGA high |
| TAGLN3\|29114 | transgelin 3 | 3.39 | 2.57E−06 | Xq28-CGA high |
| TAS2R19\|259294 | taste receptor, type 2, member 19 | 2.03 | 5.58E−05 | Xq28-CGA high |
| TCL6\|27004 | T-cell leukemia/lymphoma 6 (non-protein coding [symbol] | 18.82 | 2.79E−16 | Xq28-CGA high |
| TEKT2\|27285 | tektin 2 (testicular) | 2.94 | 3.62E−08 | Xq28-CGA high |
| TFDP3\|51270 | transcription factor Dp family, member 3 | 3.27 | 2.47E−06 | Xq28-CGA high |
| TF\|7018 | transferrin | 3.99 | 1.41E−04 | Xq28-CGA high |
| TIGD4\|201798 | tigger transposable element derived 4 | 2.31 | 1.27E−08 | Xq28-CGA high |
| TKTL2\|84076 | transketolase-like 2 | 2.13 | 4.05E−11 | Xq28-CGA high |
| TMEM195\|392636 | alkylglycerol monooxygenase | 5.68 | 1.19E−05 | Xq28-CGA high |
| TMEM22\|80723 | solute carrier family 35, member G2 | 2.26 | 8.89E−05 | Xq28-CGA high |
| TMEM25\|84866 | transmembrane protein 25 | 2.95 | 2.50E−05 | Xq28-CGA high |
| TMEM84\|283673 | | 4.13 | 3.13E−07 | Xq28-CGA high |
| TMPRSS5\|80975 | transmembrane protease, serine 5 | 2.07 | 4.57E−07 | Xq28-CGA high |
| TP53TG3B\|729355 | TP53 target 3B | 2.47 | 6.05E−06 | Xq28-CGA high |
| TPD52L1\|7164 | tumor protein D52-like 1 | 3.17 | 1.31E−07 | Xq28-CGA high |
| TPTE2P1\|646405 | transmembrane phosphoinositide 3-phosphatas [symbol] | 2.68 | 1.82E−05 | Xq28-CGA high |
| TPTE\|7179 | transmembrane phosphatase with tensin homo [symbol] | 62.08 | 1.22E−24 | Xq28-CGA high |
| TRIM17\|51127 | tripartite motif containing 17 | 2.99 | 8.69E−09 | Xq28-CGA high |
| TRIM36\|55521 | tripartite motif containing 36 | 2.06 | 9.96E−06 | Xq28-CGA high |
| TRIM54\|57159 | tripartite motif containing 54 | 2.95 | 3.90E−06 | Xq28-CGA high |
| TRIM6\|117854 | tripartite motif containing 6 | 2.29 | 4.44E−06 | Xq28-CGA high |
| TRIM9\|114088 | tripartite motif containing 9 | 2.23 | 2.74E−06 | Xq28-CGA high |
| TRPM3\|80036 | transient receptor potential cation channel, sub [symbol] | 3.46 | 6.67E−06 | Xq28-CGA high |
| TSNAX-DISC1\|100303 [symbol] | TSNAX-DISC1 readthrough (NMD candidate) | 4.38 | 1.43E−04 | Xq28-CGA high |
| TSPAN5\|10098 | tetraspanin 5 | 2.06 | 2.45E−05 | Xq28-CGA high |
| TTLL7\|79739 | tubulin tyrosine ligase-like family, member 7 | 2.28 | 0.00013952 | Xq28-CGA high |
| TUBA3C\|7278 | tubulin, alpha 3c | 12.67 | 4.33E−16 | Xq28-CGA high |
| UGT2A1\|10941 | UDP glucuronosyltransferase 2 family, polypepti [symbol] | 2.06 | 4.25E−09 | Xq28-CGA high |
| UGT8\|7368 | UDP glycosyltransferase 8 | 5.41 | 2.21E−09 | Xq28-CGA high |
| UNC80\|285175 | unc-80 homolog (C. elegans) | 2.57 | 1.40E−06 | Xq28-CGA high |
| VCX3A\|51481 | variable charge, X-linked 3A | 19.16 | 1.39E−20 | Xq28-CGA high |
| VCX3B\|425054 | variable charge, X-linked 3B | 5.79 | 1.15E−13 | Xq28-CGA high |
| VCX\|26609 | variable charge, X-linked | 13.04 | 4.05E−14 | Xq28-CGA high |
| VWDE\|221806 | von Willebrand factor D and EGF domains | 2.35 | 4.27E−06 | Xq28-CGA high |
| WDR52\|55779 | | 2.03 | 1.31E−06 | Xq28-CGA high |
| WISP3\|8838 | WNT1 inducible signaling pathway protein 3 | 2.47 | 2.26E−06 | Xq28-CGA high |
| WNK4\|65266 | WNK lysine deficient protein kinase 4 | 3.12 | 2.06E−05 | Xq28-CGA high |
| XAGE1D\|9503 | X antigen family, member 1D | 19239.33 | 1.24E−29 | Xq28-CGA high |
| XIRP2\|129446 | xin actin-binding repeat containing 2 | 2.56 | 1.64E−06 | Xq28-CGA high |
| XIST\|7503 | X inactive specific transcript (non-protein coding [symbol] | 21.76 | 9.36E−06 | Xq28-CGA high |
| XK\|7504 | X-linked Kx blood group (McLeod syndrome) | 2.62 | 3.75E−05 | Xq28-CGA high |
| ZBTB8B\|728116 | zinc finger and BTB domain containing 8B | 3.07 | 3.47E−06 | Xq28-CGA high |
| ZIC1\|7545 | Zic family member 1 | 2.51 | 6.91E−05 | Xq28-CGA high |
| ZNF157\|7712 | zinc finger protein 157 | 2.84 | 4.19E−09 | Xq28-CGA high |
| ZNF214\|7761 | zinc finger protein 214 | 2.73 | 1.45E−04 | Xq28-CGA high |
| ZNF229\|7772 | zinc finger protein 229 | 3.24 | 1.34E−07 | Xq28-CGA high |
| ZNF280A\|129025 | zinc finger protein 280A | 4.77 | 1.54E−11 | Xq28-CGA high |
| ZNF300\|91975 | zinc finger protein 300 | 3.32 | 3.73E−10 | Xq28-CGA high |
| ZNF334\|55713 | zinc finger protein 334 | 5.34 | 1.99E−07 | Xq28-CGA high |
| ZNF541\|84215 | zinc finger protein 541 | 2.08 | 3.32E−06 | Xq28-CGA high |
| ZNF556\|80032 | zinc finger protein 556 | 5.97 | 6.11E−08 | Xq28-CGA high |
| ZNF560\|147741 | zinc finger protein 560 | 29.67 | 1.43E−13 | Xq28-CGA high |
| ZNF595\|152687 | zinc finger protein 595 | 9.28 | 4.47E−18 | Xq28-CGA high |
| ZNF648\|127665 | zinc finger protein 648 | 4.46 | 1.27E−07 | Xq28-CGA high |
| ZNF660\|285349 | zinc finger protein 660 | 3.17 | 7.07E−06 | Xq28-CGA high |
| ZNF695\|57116 | zinc finger protein 695 | 2.98 | 1.12E−05 | Xq28-CGA high |
| ZNF883\|169834 | zinc finger protein 883 | 5.3 | 1.04E−10 | Xq28-CGA high |
| ZSCAN12P1\|221584 | zinc finger and SCAN domain containing 12 pseu [symbol] | 3.43 | 2.36E−07 | Xq28-CGA high |
| ZSCAN23\|222696 | zinc finger and SCAN domain containing 23 | 3.3 | 3.28E−05 | Xq28-CGA high |
| ADAMTS2\|9509 | ADAM metallopeptidase with thrombospondin t [symbol] | 2.25 | 3.02E−05 | Xq28-CGA low |
| ADARB2\|105 | adenosine deaminase, RNA-specific, B2 (non-fun [symbol] | 3.51 | 1.54E−05 | Xq28-CGA low |
| ADCY2\|108 | adenylate cyclase 2 (brain) | 12.12 | 1.54E−05 | Xq28-CGA low |
| ADRA2C\|152 | adrenoceptor alpha 2C | 4 | 6.20E−05 | Xq28-CGA low |
| AP3B2\|8120 | adaptor-related protein complex 3, beta 2 subun [symbol] | 2.33 | 1.80E−05 | Xq28-CGA low |
| ARHGAP8\|23779 | Rho GTPase activating protein 8 | 3.71 | 8.66E−08 | Xq28-CGA low |
| ARHGEF4\|50649 | Rho guanine nucleotide exchange factor (GEF) 4 | 3.29 | 2.17E−06 | Xq28-CGA low |
| ARSI\|340075 | arylsulfatase family, member I | 2.33 | 9.24E−05 | Xq28-CGA low |
| ATP1A3\|478 | ATPase, Na+/K+ transporting, alpha 3 polypeptid [symbol] | 2.75 | 1.32E−04 | Xq28-CGA low |
| B3GAT1\|27087 | beta-1,3-glucuronyltransferase 1 (glucuronosyltr [symbol] | 4.79 | 2.81E−04 | Xq28-CGA low |
| BIK\|638 | BCL2-interacting killer (apoptosis-inducing) | 2.48 | 3.49E−05 | Xq28-CGA low |
| BRSK2\|9024 | BR serine/threonine kinase 2 | 6.57 | 2.24E−08 | Xq28-CGA low |
| C10orf116\|10974 | | 2.03 | 1.21E−04 | Xq28-CGA low |
| C10orf93\|255352 | | 8.71 | 1.13E−07 | Xq28-CGA low |
| C15orf59\|388135 | | 2.63 | 2.96E−07 | Xq28-CGA low |
| C21orf121\|150142 | | 2.23 | 1.38E−05 | Xq28-CGA low |
| C5orf38\|153571 | | 16.07 | 9.72E−11 | Xq28-CGA low |
| CACNA1B\|774 | calcium channel, voltage-dependent, N type, alp [symbol] | 2.79 | 7.72E−05 | Xq28-CGA low |
| CACNA1H\|8912 | calcium channel, voltage-dependent, T type, alp [symbol] | 2.99 | 1.56E−09 | Xq28-CGA low |
| CAPG\|822 | capping protein (actin filament), gelsolin-like | 2.73 | 8.59E−06 | Xq28-CGA low |
| CBFA2T3\|863 | core-binding factor, runt domain, alpha subunit [symbol] | 2.07 | 7.68E−05 | Xq28-CGA low |
| CCDC64\|92558 | coiled-coil domain containing 64 | 2.34 | 5.12E−05 | Xq28-CGA low |
| CDHR1\|92211 | cadherin-related family member 1 | 2.47 | 3.35E−06 | Xq28-CGA low |
| CEBPA\|1050 | CCAAT/enhancer binding protein (C/EBP), alpha | 2.01 | 2.45E−06 | Xq28-CGA low |
| CHRFAM7A\|89832 | CHRNA7 (cholinergic receptor, nicotinic, alpha 7 [symbol] | 2.79 | 3.51E−04 | Xq28-CGA low |
| CHRNA7\|1139 | cholinergic receptor, nicotinic, alpha 7 (neurona [symbol] | 3.79 | 1.01E−07 | Xq28-CGA low |
| CIDEA\|1149 | cell death-inducing DFFA-like effector a | 5.47 | 0.000131166 | Xq28-CGA low |
| CPNE7\|27132 | copine VII | 3.07 | 7.29E−07 | Xq28-CGA low |
| CYS1\|192668 | cystin 1 | 2.53 | 1.72E−06 | Xq28-CGA low |
| DARC\|2532 | | 2.1 | 4.54E−05 | Xq28-CGA low |
| DGCR5\|26220 | DiGeorge syndrome critical region gene 5 (non-p [symbol] | 4.08 | 3.25E−10 | Xq28-CGA low |
| DKFZp779M0652\|374387 | | 2.25 | 1.43E−08 | Xq28-CGA low |
| DMRT2\|10655 | doublesex and mab-3 related transcription facto [symbol] | 3.59 | 5.31E−05 | Xq28-CGA low |
| DOC2B\|8447 | double C2-like domains, beta | 2.14 | 2.60E−04 | Xq28-CGA low |
| DPYSL4\|10570 | dihydropyrimidinase-like 4 | 2.48 | 9.03E−05 | Xq28-CGA low |
| DUSP8\|1850 | dual specificity phosphatase 8 | 2.02 | 0.000253945 | Xq28-CGA low |
| DYNLRB2\|83657 | dynein, light chain, roadblock-type 2 | 2.15 | 6.20E−05 | Xq28-CGA low |
| EMID2\|136227 | collagen, type XXVI, alpha 1 | 2.74 | 2.90E−07 | Xq28-CGA low |
| FAM171A1\|221061 | family with sequence similarity 171, member A1 | 2.19 | 0.000114552 | Xq28-CGA low |
| FAM19A5\|25817 | family with sequence similarity 19 (chemokine ( [symbol] | 5.61 | 5.22E−06 | Xq28-CGA low |
| FIBCD1\|84929 | fibrinogen C domain containing 1 | 3.2 | 4.22E−05 | Xq28-CGA low |
| FOXF2\|2295 | forkhead box F2 | 2.38 | 4.92E−05 | Xq28-CGA low |
| FOXQ1\|94234 | forkhead box Q1 | 2.15 | 9.40E−05 | Xq28-CGA low |
| FZD8\|8325 | frizzled class receptor 8 | 2.12 | 2.84E−04 | Xq28-CGA low |
| GABBR2\|9568 | gamma-aminobutyric acid (GABA) B receptor, 2 | 2.41 | 4.17E−08 | Xq28-CGA low |
| GABRA5\|2558 | gamma-aminobutyric acid (GABA) A receptor, al [symbol] | 4.93 | 2.66E−07 | Xq28-CGA low |
| GABRG3\|2567 | gamma-aminobutyric acid (GABA) A receptor, ga [symbol] | 12.79 | 9.85E−05 | Xq28-CGA low |
| GAS6\|2621 | growth arrest-specific 6 | 2.23 | 7.12E−07 | Xq28-CGA low |
| GCNT4\|51301 | glucosaminyl (N-acetyl) transferase 4, core 2 | 2.51 | 1.70E−05 | Xq28-CGA low |
| GMPR\|2766 | guanosine monophosphate reductase | 3.05 | 1.49E−05 | Xq28-CGA low |
| GNAL\|2774 | guanine nucleotide binding protein (G protein), [symbol] | 4.64 | 1.17E−09 | Xq28-CGA low |
| GNAO1\|2775 | guanine nucleotide binding protein (G protein), [symbol] | 2.9 | 1.15E−04 | Xq28-CGA low |
| GOLGA7B\|401647 | golgin A7 family, member B | 2.13 | 0.000106118 | Xq28-CGA low |
| GRID1\|2894 | glutamate receptor, ionotropic, delta 1 | 2.51 | 9.48E−10 | Xq28-CGA low |
| GSG1L\|146395 | GSG1-like | 3.08 | 2.72E−06 | Xq28-CGA low |
| HAR1A\|768096 | highly accelerated region 1A (non-protein coding [symbol] | 3.32 | 5.76E−12 | Xq28-CGA low |
| HAR1B\|768097 | highly accelerated region 1B (non-protein coding [symbol] | 3.57 | 1.45E−10 | Xq28-CGA low |
| HCN2\|610 | hyperpolarization activated cyclic nucleotide-gat [symbol] | 2.25 | 1.88E−04 | Xq28-CGA low |
| HRH3\|11255 | histamine receptor H3 | 2.92 | 8.37E−07 | Xq28-CGA low |
| HS3ST2\|9956 | heparan sulfate (glucosamine) 3-O-sulfotransfer [symbol] | 3.09 | 1.23E−05 | Xq28-CGA low |
| HSPB8\|26353 | heat shock 22 kDa protein 8 | 3.83 | 1.67E−05 | Xq28-CGA low |
| HUNK\|30811 | hormonally up-regulated Neu-associated kinase | 2.82 | 0.000262353 | Xq28-CGA low |
| IRX1\|79192 | iroquois homeobox 1 | 6.16 | 2.43E−06 | Xq28-CGA low |
| IRX2\|153572 | iroquois homeobox 2 | 9.36 | 1.54E−10 | Xq28-CGA low |
| ITGB1BP3\|27231 | nicotinamide riboside kinase 2 | 6.72 | 2.16E−06 | Xq28-CGA low |
| KCNH4\|23415 | potassium voltage-gated channel, subfamily H (e [symbol] | 2.45 | 1.31E−06 | Xq28-CGA low |
| KIAA1543\|57662 | calmodulin regulated spectrin-associated protei [symbol] | 3.57 | 1.60E−06 | Xq28-CGA low |
| KIF1A\|547 | kinesin family member 1A | 3.31 | 2.82E−05 | Xq28-CGA low |
| KIF26A\|26153 | kinesin family member 26A | 3.24 | 9.53E−15 | Xq28-CGA low |
| KNDC1\|85442 | kinase non-catalytic C-lobe domain (KIND) conta [symbol] | 7.08 | 5.98E−16 | Xq28-CGA low |
| L1CAM\|3897 | L1 cell adhesion molecule | 3.61 | 1.67E−04 | Xq28-CGA low |
| LCE2A\|353139 | late cornified envelope 2A | 5.9 | 2.62E−04 | Xq28-CGA low |
| LIMS2\|55679 | LIM and senescent cell antgen-like domains 2 | 2.04 | 8.94E−06 | Xq28-CGA low |
| LOC100127888\|100127888 | | 2.11 | 2.25E−04 | Xq28-CGA low |
| LOC284837\|284837 | | 2.08 | 2.81E−06 | Xq28-CGA low |
| LOC338651\|338651 | | 2.69 | 8.61E−07 | Xq28-CGA low |
| LOC389458\|389458 | | 2.08 | 2.38E−04 | Xq28-CGA low |
| LOC440925\|440925 | | 2.4 | 6.65E−05 | Xq28-CGA low |
| LOC80054\|80054 | | 2.14 | 2.35E−08 | Xq28-CGA low |
| LONRF3\|79836 | LON peptidase N-terminal domain and ring finge [symbol] | 2.61 | 2.90E−05 | Xq28-CGA low |
| LRRC26\|389816 | leucine rich repeat containing 26 | 4.44 | 4.41E−05 | Xq28-CGA low |
| LTF\|4057 | lactotransferrin | 2.53 | 0.000291253 | Xq28-CGA low |
| MAGED4B\|81557 | melanoma antigen family D, 4B | 4.16 | 5.73E−05 | Xq28-CGA low |
| MAGED4\|728239 | melanoma antigen family D, 4 | 4.37 | 6.71E−05 | Xq28-CGA low |
| MATK\|4145 | megakaryocyte-associated tyrosine kinase | 2.86 | 9.34E−07 | Xq28-CGA low |
| MC1R\|4157 | melanocortin 1 receptor (alpha melanocyte stim [symbol] | 2.42 | 3.56E−05 | Xq28-CGA low |
| MEGF6\|1953 | multiple EGF-like-domains 6 | 4.12 | 9.53E−12 | Xq28-CGA low |
| MEIS3\|56917 | Meis homeobox 3 | 2.14 | 1.80E−04 | Xq28-CGA low |
| MNX1\|3110 | motor neuron and pancreas homeobox 1 | 7.94 | 5.05E−07 | Xq28-CGA low |
| NPDC1\|56654 | neural proliferation, differentiation and control, [symbol] | 2.07 | 7.67E−05 | Xq28-CGA low |
| NPTXR\|23467 | neuronal pentraxin receptor | 3.04 | 1.35E−05 | Xq28-CGA low |
| NTN1\|9423 | netrin 1 | 3.61 | 1.47E−08 | Xq28-CGA low |
| NTNG2\|84628 | netrin G2 | 2.41 | 5.55E−08 | Xq28-CGA low |
| OCA2\|4948 | oculocutaneous albinism II | 21.65 | 1.15E−05 | Xq28-CGA low |
| OLFM1\|10439 | olfactomedin 1 | 14.79 | 1.59E−15 | Xq28-CGA low |
| OTUD7A\|161725 | OTU deubiquitinase 7A | 2.61 | 1.09E−07 | Xq28-CGA low |
| PANX2\|56666 | pannexin 2 | 3.37 | 4.51E−07 | Xq28-CGA low |
| PCBP3\|54039 | poly(rC) binding protein 3 | 3.31 | 1.77E−06 | Xq28-CGA low |
| PDE9A\|5152 | phosphodiesterase 9A | 5.8 | 8.69E−09 | Xq28-CGA low |
| PGBD5\|79605 | piggyBac transposable element derived 5 | 2.11 | 1.56E−06 | Xq28-CGA low |
| PHF21B\|112885 | PHD finger protein 21B | 3.98 | 3.59E−06 | Xq28-CGA low |
| PHYHD1\|254295 | phytanoyl-CoA dioxygenase domain containing [symbol] | 2.25 | 5.66E−05 | Xq28-CGA low |
| PLAC2\|257000 | tissue differentiation-indudng non-protein codin [symbol] | 6.62 | 1.05E−07 | Xq28-CGA low |
| PMEPA1\|56937 | prostate transmembrane protein, androgen indu [symbol] | 2.1 | 5.96E−05 | Xq28-CGA low |
| PNMA6A\|84968 | paraneoplastic Ma antigen family member 6A | 3.11 | 4.76E−08 | Xq28-CGA low |
| PRODH\|5625 | proline dehydrogenase (oxidase) 1 | 3 | 5.90E−06 | Xq28-CGA low |
| PRR5-ARHGAP8\|5531 [symbol] | PRR5-ARHGAP8 readthrough | 9.56 | 2.42E−05 | Xq28-CGA low |
| PTK6\|5753 | protein tyrosine kinase 6 | 2.93 | 2.17E−06 | Xq28-CGA low |
| RADIL\|55698 | Ras association and DIL domains | 4.59 | 1.96E−08 | Xq28-CGA low |
| RPS6KA2\|6196 | ribosomal protein S6 kinase, 90 kDa, polypeptide [symbol] | 2.38 | 1.22E−05 | Xq28-CGA low |
| SDK1\|221935 | sidekick cell adhesion molecule 1 | 2.27 | 8.66E−06 | Xq28-CGA low |
| SFTPD\|6441 | surfactant protein D | 2.12 | 0.000262896 | Xq28-CGA low |
| SH3GL2\|6456 | SH3-domain GRB2-like 2 | 4.54 | 2.12E−04 | Xq28-CGA low |
| SIGLEC8\|27181 | sialic acid binding Ig-like lectin 8 | 2.58 | 0.000344343 | Xq28-CGA low |
| SLC16A6\|9120 | solute carrier family 16, member 6 | 4.38 | 2.79E−06 | Xq28-CGA low |
| SLC24A4\|123041 | solute carrier family 24 (sodium/potassium/calci [symbol] | 3.39 | 3.08E−05 | Xq28-CGA low |
| SLC39A12\|221074 | solute carrier family 39 (zinc transporter), memb [symbol] | 2.2 | 0.000277556 | Xq28-CGA low |
| SLPI\|6590 | secretory leukocyte peptidase inhibitor | 3.31 | 0.000158902 | Xq28-CGA low |
| SNCB\|6620 | synuclein, beta | 2.2 | 0.000254582 | Xq28-CGA low |
| SOX1\|6656 | SRY (sex determining region Y)-box 1 | 7.73 | 1.18E−08 | Xq28-CGA low |
| SP5\|389058 | Sp5 transcription factor | 2.72 | 7.66E−05 | Xq28-CGA low |
| SYNC\|81493 | syncoilin, intermediate filament protein | 2.17 | 1.99E−05 | Xq28-CGA low |
| TCERG1L\|256536 | transcription elongation regulator 1-like | 2.67 | 1.42E−04 | Xq28-CGA low |
| TFF3\|7033 | trefoil factor 3 (intestinal) | 4.18 | 1.26E−06 | Xq28-CGA low |
| TMPRSS13\|84000 | transmembrane protease, serine 13 | 6.69 | 6.70E−09 | Xq28-CGA low |
| TWIST2\|117581 | twist family bHLH transcription factor 2 | 3.28 | 7.49E−07 | Xq28-CGA low |
| USP43\|124739 | ubiquitin specific peptidase 43 | 6.39 | 4.55E−08 | Xq28-CGA low |
| VIPR1\|7433 | vasoactive intestinal peptide receptor 1 | 2.1 | 0.000161869 | Xq28-CGA low |
| WNT9B\|7484 | wingless-type MMTV integration site family, me [symbol] | 3.67 | 3.57E−06 | Xq28-CGA low |
| ZNF423\|23090 | zinc finger protein 423 | 2.29 | 3.19E−06 | Xq28-CGA low |

[symbol]

indicates data missing or illegible when filed

TABLE 4
Overlapping genes between no benefit/clinical benefit groups and TCGA Xq28-CGA-high/low subsets

| | TCCGA Xq28-CGA high | TCCGA Xq28-CGA low |
|---|---|---|
| | (n = 559) | (n = 130) |
| No benefit | ABCA8, AKAP6, ANGPT1, ANKFN1, ANKRD20A3, ANKRD20A4, | 0 |
| (n = 975) | ANKRD45, ANKRD7, ANO3, ATP1B2, B3GALNT1, C12orf56, C1QTNF3, | |
| | C21orf90, C2orf66, C3orf30, C4orf19, CABP4, CADM4, CASP12, CCDC136, | |
| | CFHR4, CFI, CNDP1, CSAG1, CSAG2, CSAG3, CYP26A1, DCAF4L2, DDX25, | |
| | EFHC2, EGF, ELOVL2, EYA1, FAM106A, FAM81B, FGF2, FRAS1, | |
| | GABRA3, GABRB1, GABRG2, GABRQ, GAGE12D, GALNT8, GAP43, GDNF, | |
| | GNGT1, GPR158, GRIA2, GYPE, HHATL, HMGA2, HOXA2, HOXA3, HOXD10, | |
| | HOXD11, HOXD13, IGFN1, IL13RA2, ISL2, KCNC2, KCNJ10, KCNMB2, | |
| | KLF17, KLHL13, LCTL, LGALS12, LMOD2, LRP4, MAGEA1, MAGEA11, | |
| | MAGEA12, MAGEA2, MAGEA3, MAGEA6, MAGEA8, | |
| | MAGEA9B, MAGEB2, MAGEC1, MAGEC2, MAGEC3, MEGF10, | |
| | MMEL1, MMP16, MRGPRX3, MYH13, NEB, NECAB1, NLRP4, NPY6R, NXPH1, | |
| | OLFM3, OR56B4, PCDHB18, PCSK1, PEX5L, PI15, PLAC1, POU5F1B, PPP1R1C, | |
| | PPP1R9A, PSG9, RAPGEF4, RBM20, RFPL4B, RND2, SCN2A, SERPINA5, | |
| | SLC10A4, SLC18A1, SLC30A3, SLCO1A2, SOSTDC1, SPAG17, | |
| | SPP1, SPRY4, SSX6, SV2A, SYT1, SYTL5, TEKT2, TPD52L1, | |
| | TRIM9, TRPM3, TSPAN5, WNK4, XIRP2, XIST, ZNF334, ZNF541 | |
| Clinical benefit | 0 | ADCY2, ATP1A3, BIK, C5orf38, CPNE7, |
| (n = 428) | | GOLGA7B, HS3ST2, IRX2, LRRC26, |
| | | MNX1, SIGLEC8, SLC24A4, USP43 |

The following references were cited in this specification.

### REFERENCES

Anders, S., Pyl, P. T., and Huber, W. (2015). HTSeq—a Python framework to work with highthroughput sequencing data. Bioinformatics (Oxford, England) 31, 166-169.
Angelova, M., Charoentong, P., Hackl, H., Fischer, M. L., Snajder, R., Krogsdam, A. M., Waldner, M. J., Bindea, G., Mlecnik, B., Galon, J., et al. (2015). Characterization of the immunophenotypes and antigenomes of colorectal cancers reveals distinct tumor escape mechanisms and novel targets for immunotherapy. Genome biology 16, 64.
Apetoh, L., Ghiringhelli, F., Tesniere, A., Obeid, M., Ortiz, C., Criollo, A., Mignot, G., Maiuri, M. C., Ullrich, E., Saulnier, P., et al. (2007). Toll-like receptor 4-dependent contribution of the immune system to anticancer chemotherapy and radiotherapy. Nat Med 13, 1050-1059.
Avalos, A. M., Kiefer, K., Tian, J., Christensen, S., Shlomchik, M., Coyle, A. J., and Marshak-Rothstein, A. (2010). RAGE-independent autoreactive B cell activation in response to chromatin and HMGB1/DNA immune complexes. Autoimmunity 43, 103-110.
Aimi, F., Scolyer, R. A., Rumcheva, P., Moncrieff, M., Murali, R., McCarthy, S. W., Saw, R. P., and Thompson, J. F. (2012). Tumor-infiltrating lymphocyte grade is an independent predictor of sentinel lymph node status and survival in patients with cutaneous melanoma. Journal of clinical oncology: official journal of the American Society of Clinical Oncology 30, 2678-2683.
Barrow, C., Browning, J., MacGregor, D., Davis, I. D., Sturrock, S., Jungbluth, A. A., and Cebon, J. (2006). Tumor antigen expression in melanoma varies according to antigen and stage. Clin Cancer Res 12, 764-771.
Bredenbeck, A., Hollstein, V. M., Trefzer, U., Sterry, W., Walden, P., and Losch, F. O. (2008). Coordinated expression of clustered cancer/testis genes encoded in a large inverted repeat DNA structure. Gene 415, 68-73.
Cancer Genome Atlas, N. (2015). Genomic Classification of Cutaneous Melanoma. Cell 161, 1681-1696.
Carter, S. L., Cibulskis, K., Helman, E., McKenna, A., Shen, H., Zack, T., Laird, P. W., Onofrio, R. C., Winckler, W., Weir, B. A., et al. (2012). Absolute quantification of somatic DNA alterations in human cancer. Nature biotechnology 30, 413-421.
Chen, Y. T., Panarelli, N. C., Piotti, K. C., and Yantiss, R. K. (2014). Cancer-testis antigen expression in digestive tract carcinomas: frequent expression in esophageal squamous cell carcinoma and its precursor lesions. Cancer immunology research 2, 480-486.
Coulie, P. G., Van den Eynde, B. J., van der Bruggen, P., and Boon, T. (2014). Tumour antigens recognized by T lymphocytes: at the core of cancer immunotherapy. Nature reviews Cancer 14, 135-146.
De Plaen, E., Arden, K., Traversari, C., Gaforio, J. J., Szikora, J. P., De Smet, C., Brasseur, F., van der Bruggen, P., Lethe, B., Lurquin, C., et aL (1994). Structure, chromosomal localization, and expression of 12 genes of the MAGE family. Immunogenetics 40, 360-369.
Dobin, A., Davis, C. A., Schlesinger, F., Drenkow, J., Zaleski, C., Jha, S., Batut, P., Chaisson, M., and Gingeras, T. R. (2013). STAR: ultrafast universal RNA-seq aligner. Bioinformatics (Oxford, England) 29, 15-21.
Doyle, J. M., Gao, J., Wang, J., Yang, M., and Potts, P. R (2010). MAGE-RING protein complexes comprise a family of E3 ubiquitin ligases. Molecular cell 39, 963-974.
Galluzzi, L., Buque, A., Kepp, O., Zitvogel, L., and Kroemer, G. (2017). Immunogenic cell death in cancer and infectious disease. Nature reviews Immunology 17, 97-111.
Gao, J., Shi, L. Z., Zhao, H., Chen, J., Xiong, L., He, Q., Chen, T., Roszik, J., Bernatchez, C., Woodman, S. E, et aL (2016). Loss of IFN-gamma Pathway Genes in Tumor Cells as a Mechanism of Resistance to Anti-CTLA-4 Therapy. Cell 167, 397-404 e399.
Gumireddy, K., Li, A., Kossenkov, A. V., Sakurai, M., Yan, J., Li, Y., Xu, H., Wang, J., Zhang, P. J., Zhang, L., et al. (2016). The mRNA-edited form of GABRA3 suppresses GABRA3-mediated Akt activation and breast cancer metastasis. Nature communications 7, 10715.
Hodi, F. S., O'Day, S. J., McDermott, D. F., Weber, R. W., Sosman, J. A., Haanen, J. B., Gonzalez, R., Robert, C., Schadendorf, D., Hassel, J. C., et al. (2010). Improved survival with ipilimumab in patients with metastatic melanoma. N Engl J Med 363, 711-723.
Hugo, W., Zaretsky, J. M., Sun, L., Song, C., Moreno, B. H., Hu-Lieskovan, S., Berent-Maoz, B., Pang, J., Chmielowski, B., Cherry, G., et aL (2016). Genomic and Transcriptomic Features of Response to Anti-PD-1 Therapy in Metastatic Melanoma. Cell 165, 35-44.
Ivanov, S., Dragoi, A. M., Wang, X., Dallacosta, C., Louten, J., Musco, G., Sitia, G., Yap, G. S., Wan, Y., Biron, C. A., et al. (2007). A novel role for HMGB1 in TLR9-mediated inflammatory responses to CpG-DNA. Blood 110, 1970-1981.
Kalluri, R., and Weinberg, R. A. (2009). The basics of epithelial-mesenchymal transition. J Clin Invest 119, 1420-1428.
Ladoire, S., Penault-Llorca, F., Senovilla, L., Dalban, C., Enot, D., Locher, C., Prada, N., Poirier-Colame, V., Chaba, K., Amould, L., et al. (2015). Combined evaluation of LC3B puncta and HMGB1 expression predicts residual risk of relapse after adjuvant chemotherapy in breast cancer. Autophagy 11, 1878-1890.
Lee, A. K., and Potts, P. R. (2017). A comprehensive guide to the MAGE family of ubiquitin ligases. Journal of molecular biology.
Li, B., and Dewey, C. N. (2011). RSEM: accurate transcript quantification from RNA-Seq data with or without a reference genome. BMC bioinformatics 12, 323.
Li, G., Liang, X., and Lotze, M. T. (2013). HMGB1: The Central Cytokine for All Lymphoid Cells. Frontiers in immunology 4, 68.
Li, Y., Wang, L. X., Yang, G., Hao, F., Urba, W. J., and Hu, H. M. (2008). Efficient crosspresentation depends on autophagy in tumor cells. Cancer research 68, 6889-6895.
Mi, H., Poudel, S., Muruganujan, A., Casagrande, J. T., and Thomas, P. D. (2016). PANTHER version 10: expanded protein families and functions, and analysis tools. Nucleic acids research 44, D336-342.
Michaud, M., Martins, I., Sukkurwala, A. Q., Adjemian, S., Ma, Y., Pellegatti, P., Shen, S., Kepp, O., Scoazec, M., Mignot, G., et aL (2011). Autophagy-dependent anticancer immune responses induced by chemotherapeutic agents in mice. Science 334, 1573-1577.
Palucka, K., and Banchereau, J. (2014). SnapShot: Cancer Vaccines. Cell 157, 516-516 e511. Pedicord, V. A., Montalvo, W., Leiner, I. M., and Allison, J. P. (2011). Single dose of anti-CTLA-4 enhances CD8+ T-cell memory formation, function, and maintenance. Proceedings of the National Academy of Sciences of the United States of America 108, 266-271.
Pineda, C. T., Ramanathan, S., Fon Tacer, K., Weon, J. L., Potts, M. B., Ou, Y. H., White, M. A., and Potts, P. R. (2015). Degradation of AMPK by a cancer-specific ubiquitin ligase. Cell 160, 715-728.
Pu, Y., Xu, M., Liang, Y., Yang, K., Guo, Y., Yang, X., and Fu, Y. X. (2016). Androgen receptor antagonists compromise T cell response against prostate cancer leading to early tumor relapse. Science translational medicine 8, 333ra347.
Riaz, N., Havel, J. J., Kendall, S. M., Makarov, V., Walsh, L. A., Desrichard, A., Weinhold, N., and Chan, T. A. (2016). Recurrent SERPINB3 and SERPINB4 mutations in patients who respond to anti-CTLA4 immunotherapy. Nature genetics 48, 1327-1329.
Roeder, C., Schuler-Thurner, B., Berchtold, S., Vieth, G., Driesch, P., Schuler, G., and Luftl, M. (2005). MAGE-A3 is a frequent tumor antigen of metastasized melanoma. Archives of dermatological research 296, 314-319.
Saiag, P., Gutzmer, R., Ascierto, P. A., Maio, M., Grob, J. J., Murawa, P., Dreno, B., Ross, M., Weber, J., Hauschild, A., et aL (2016). Prospective assessment of a gene signature potentially predictive of clinical benefit in metastatic melanoma patients following MAGE-A3 immunotherapeutic (PREDICT). Annals of oncology: official journal of the European Society for Medical Oncology/ESMO 27, 1947-1953.
Scaffidi, P., Misteli, T., and Bianchi, M. E. (2002). Release of chromatin protein HMGB1 by necrotic cells triggers inflammation. Nature 418, 191-195.
Simpson, A. J., Caballero, 01., Jungbluth, A., Chen, Y. T., and Old, L. J. (2005). Cancer/testis antigens, gametogenesis and cancer. Nature reviews Cancer 5, 615-625.
Snyder, A., Makarov, V., Merghoub, T., Yuan, J., Zaretsky, J. M., Desrichard, A., Walsh, L. A., Postow, M. A., Wong, P., Ho, T. S., et al. (2014). Genetic basis for clinical response to CTLA-4 blockade in melanoma. N Engl J Med 371, 2189-2199.
Tang, D., Kang, R., Coyne, C. B., Zeh, H. J., and Lotze, M. T. (2012). PAMPs and DAMPs: signal Os that spur autophagy and immunity. Immunological reviews 249, 158-175.
Tang, D., Kang, R., Livesey, K. M., Cheh, C. W., Farkas, A., Loughran, P., Hoppe, G., Bianchi, M. E., Tracey, K. J., Zeh, H. J., 3rd, et al. (2010). Endogenous HMGB1 regulates autophagy. The Journal of cell biology 190, 881-892.
Tian, J., Avalos, A. M., Mao, S. Y., Chen, B., Senthil, K., Wu, H., Parroche, P., Drabic, S., Golenbock, D., Sirois, C., et al. (2007). Toll-like receptor 9-dependent activation by DNAcontaining immune complexes is mediated by HMGB1 and RAGE. Nat Immunol 8, 487-496.
Tittarelli, A., Gonzalez, F. E., Pereda, C., Mora, G., Munoz, L., Saffie, C., Garcia, T., Diaz, D., Falcon, C., Hermoso, M., et aL (2012). Toll-like receptor 4 gene polymorphism influences dendritic cell in vitro function and clinical outcomes in vaccinated melanoma patients. Cancer immunology, immunotherapy: CII 61, 2067-2077.
Van Allen, E. M., Miao, D., Schilling, B., Shukla, S. A., Blank, C., Zimmer, L., Sucker, A., Hillen, U., Foppen, M. H., Goldinger, S. M., et aL (2015). Genomic correlates of response to CTLA-4 blockade in metastatic melanoma. Science 350, 207-211.
van der Bruggen, P., Traversari, C., Chomez, P., Lurquin, C., De Plaen, E., Van den Eynde, B., Knuth, A., and Boon, T. (1991). A gene encoding an antigen recognized by cytolytic T lymphocytes on a human melanoma. Science 254, 1643-1647.
Van Der Bruggen, P., Zhang, Y., Chaux, P., Stroobant, V., Panichelli, C., Schultz, E. S., Chapiro, J., Van Den Eynde, B. J., Brasseur, F., and Boon, T. (2002). Tumor-specific shared antigenic peptides recognized by human T cells. Immunological reviews 188, 51-64.
Vansteenkiste, J. F., Cho, B. C., Vanakesa, T., De Pas, T., Zielinski, M., Kim, M. S., Jassem, J., Yoshimura, M., Dahabreh, J., Nakayama, H., et al. (2016). Efficacy of the MAGE-A3 cancer immunotherapeutic as adjuvant therapy in patients with resected MAGE-A3-positive nonsmall-cell lung cancer (MAGRIT): a randomised, double-blind, placebo-controlled, phase 3 trial. The lancet oncology 17, 822-835.
Weber, J. S., Gibney, G., Sullivan, R. J., Sosman, J. A., Slingluff, C. L., Jr., Lawrence, D. P., Logan, T. F., Schuchter, L. M., Nair, S., Fecher, L., et aL (2016). Sequential administration of nivolumab and ipilimumab with a planned switch in patients with advanced melanoma (CheckMate 064): an open-label, randomised, phase 2 trial. The lancet oncology 17, 943-955.
Wolchok, J. D., Kluger, H., Callahan, M. K., Postow, M. A., Rizvi, N. A., Lesokhin, A. M., Segal, N. H., Ariyan, C. E., Gordon, R. A., Reed, K., et al. (2013). Nivolumab plus ipilimumab in advanced melanoma. N Engl J Med 369, 122-133.
Yanai, H., Ban, T., Wang, Z., Choi, M. K., Kawamura, T., Negishi, H., Nakasato, M., Lu, Y., Hangai, S., Koshiba, R., et aL (2009). HMGB proteins function as universal sentinels for nucleic-acid-mediated innate immune responses. Nature 462, 99-103.
Yatim, N., Cullen, S., and Albert, M. L. (2017). Dying cells actively regulate adaptive immune responses. Nature reviews Immunology 17, 262-275.
Yuan, J., Adamow, M., Ginsberg, B. A., Rasalan, T. S., Ritter, E., Gallardo, H. F., Xu, Y., Pogoriler, E., Terzulli, S. L., Kuk, D., et aL (2011). Integrated NY-ESO-1 antibody and CD8+ T-cell responses correlate with clinical benefit in advanced melanoma patients treated with ipilimumab. Proceedings of the National Academy of Sciences of the United States of America 108, 16723-16728.
Zaretsky, J. M., Garcia-Diaz, A., Shin, D. S., Escuin-Ordinas, H., Hugo, W., Hu-Lieskovan, S., Torrejon, D. Y., Abril-Rodriguez, G., Sandoval, S., Barthly, L., et al. (2016). Mutations Associated with Acquired Resistance to PD-1 Blockade in Melanoma. N Engl J Med 375, 819-829.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

1. A method of determining whether inhibition of cytotoxic T-lymphocyte-associated protein 4 (CTLA4) in a subject with melanoma will result in clinical benefit in the subject comprising:
obtaining a test sample from a subject having or at risk of developing melanoma;
determining the expression level of at least one melanoma-associated gene in the test sample;
comparing the expression level of the melanoma-associated gene in the test sample with the expression level of the melanoma-associated gene in a reference sample; and
determining whether CTLA4 blockade will inhibit melanoma in the subject if the expression level of the melanoma-associated gene in the test sample is differentially expressed as compared to the level of the melanoma-associated gene in the reference sample; or
a method of determining whether inhibition of CTLA4 in a subject with melanoma will result in clinical benefit in the subject comprising:
obtaining a test sample from a subject having or at risk of developing melanoma;
determining the expression level of at least one melanoma-associated gene in the test sample;
comparing the expression level of the melanoma-associated gene in the test sample with the expression level of a housekeeping gene in a reference sample; and
determining whether CTLA4 blockade will inhibit melanoma in the subject if the expression level of the melanoma-associated gene in the test sample is differentially expressed as compared to the level of the housekeeping gene in the reference sample; or
a method to predict a response of a subject with cancer to a CTLA4 therapy, the method comprising:
(a) assaying for
(i) aberrant expression of at least one resistant cancer-associated gene or miRNA in a biological sample from the subject, wherein the aberrant expression of the at least one resistant cancer-associated gene or miRNA is not present in the biological sample, or
(ii) aberrant expression of at least one beneficial cancer-associated gene or miRNA in a
biological sample from the subject, wherein the aberrant expression of the at least one beneficial cancer-associated gene or miRNA is present in the biological sample, and
(b) predicting a response of the subject with cancer to a CTLA4 therapy to be positive based on the assaying.

2. The method of claim 1, wherein the test sample is obtained from the melanoma tissue or from the tumor microenvironment or from tumor-infiltrating immune cells.

3. The method of claim 1, wherein clinical benefit in the subject comprises complete or partial response as defined by response evaluation criteria in solid tumors (RECIST), stable disease as defined by RECIST, or long-term survival in spite of disease progression or response as defined by irRC criteria.

4. The method of claim 1, wherein the test sample is obtained from the melanoma, and wherein the melanoma-associated gene comprises a gene on chromosome Xq28.

5. The method of claim 1, wherein the test sample is obtained from the melanoma, wherein the melanoma-associated gene comprises a cancer germline antigen (CGA) gene; and
determining that inhibition of CTLA4 in a subject with melanoma will not result in clinical benefit in the subject if the expression level of the CGA gene in the test sample is higher than the level of the CGA gene in the reference sample.

6. The method of claim 5, wherein the CGA gene comprises melanoma-associated antigen 2 (MAGEA2), MAGEA3, MAGEA6, MAGEA12, chondrosarcoma associated gene 1 (CSAG1), CSAG2, CSAG3 or CSAG4.

7. The method of claim 6, wherein the CGA gene is hypomethylated.

8.-9. (canceled)

10. The method of claim 1, wherein the test sample is obtained from the melanoma, wherein the melanoma-associated gene comprises a pregnancy-specific glycoprotein (PSG) gene, a γ-aminobutyric acid (GABA) A receptor gene, an epithelial-to-mesenchymal transition gene, an embryonic development/differentiation gene, an angiogenesis gene, or an extracellular matrix (ECM) gene; and
determining that inhibition of CTLA4 in a subject with melanoma will not result in clinical benefit in the subject if the expression level of the PSG gene, GABA A receptor gene, epithelial-to-mesenchymal transition gene, embryonic development/differentiation gene, angiogenesis gene, or extracellular matrix gene in the test sample is higher than the level of the corresponding gene in the reference sample.

11.-12. (canceled)

13. The method of claim 10, wherein the GABA A receptor gene comprises gamma-aminobutyric acid type A receptor alpha 3 subunit (GABRA3), gamma-aminobutyric acid type A receptor beta 1 subunit (GABRB1), GABRB2, gamma-aminobutyric acid type A receptor gamma 2 subunit (GABRG2), gamma-aminobutyric acid type A receptor theta subunit (GABRQ), or gamma-aminobutyric acid type A receptor rho 1 subunit (GABRR1).

14. The method of claim 10, wherein the epithelial-to-mesenchymal transition gene comprises claudin 1 (CLDN1), CLDN2, eyes absent homolog 1 (EYA1), snail family zinc finger 1 (SNAI1), transforming growth factor beta 2 (TGFB2), or wingless-type MMTV integration site family member 3 (WNT3).

15.-17. (canceled)

18. The method of claim 1, wherein the test sample is obtained from the melanoma, wherein the melanoma-associated gene comprises MAGEA2, CSAG4, MAGEA2B, RP11-215P9, MAGEA12, CSAG1, GABRA3, CSAG3, makorin ring finger protein 9 (MKRN9P), keratin 8 pseudogene 8 (KRT8P8), MAGEA6, EYA1, CSAG2, RP11-379D21.3, MAGEC1, RP1-273G13.1, MAGEA3, miR-218-1, PSG11, X-inactive specific transcript (XIST), RP11-360D2.1, pregnancy specific beta-1-glycoprotein 10 pseudogene (PSG10P), miR-1262, tachykinin 3 (TAC3), PSG8, heat shock protein family B (small) member 3 (HSPB3), gap junction protein beta-6 (GJB6), PSG6, GABRQ, MAGEA1, MAGEA11 or MAGEA9B; and
determining that inhibition of CTLA4 in a subject with melanoma will not result in clinical benefit in the subject if the expression level of the melanoma-associated gene in the test sample is higher than the level of the corresponding gene in the reference sample.

19. The method of claim 1, wherein the test sample is obtained from the melanoma, and wherein the melanoma-associated gene comprises micro ribonucleic acid-211 (miR-211), miR-513A2, miR-185, or TRPM1; and
determining that inhibition of CTLA4 in a subject with melanoma will result in clinical benefit in the subject if the expression level of miR-211, miR-513A2, miR-185, or TRPM1 in the test sample is higher than the level of miR-211, miR-513A2, miR-185, or TRPM1, respectively, in the reference sample.

20. The method of claim 1, wherein the test sample is obtained from the melanoma, and wherein the melanoma-associated gene comprises transient receptor potential cation channel subfamily M member 1 (TRPM1); and
determining that inhibition of CTLA4 in a subject with melanoma will result in clinical benefit in the subject if the expression level of TRPM1 in the test sample is higher than the level of TRPM1 in the reference sample.

21. The method of claim 1, wherein the test sample is obtained from the melanoma or the infiltrating immune cells, and wherein the melanoma-associated gene comprises miR-211, MAGEA2, MAGEA3, MAGEA6, MAGEA12, CSAG1, CSAG2, CSAG3, CSAG4; and
determining that inhibition of CTLA4 in a subject with melanoma will not result in clinical benefit in the subject if the expression level of miR-211 in the test sample is lower than the level of miR-211 in the reference sample and if the expression level of MAGEA2, MAGEA3, MAGEA6, MAGEA12, CSAG1, CSAG2, CSAG3, and CSAG4 in the test sample is higher than the level of the corresponding gene in the reference sample.

22. The method of claim 1, wherein the test sample is obtained from the melanoma, and wherein the melanoma-associated gene comprises miR-211 and one or more of CD2, CD6, CXCL13, CD3D, CD3E, CD3G, LCK, T cell receptor alpha gene, T cell receptor beta gene, CD28, ICOS, EOMES, IL2RB, FASLG, SLAMF6, GNLY, GZMA, GZMB, GZMH, GZMK, PRF1, PTCRA, CD19, CD72, FCRL1/3, MS4A1, CTLA4, LAG3, FCRL1, FCRL3, CD5L, SIGLEC8, or FAIM3/TOSO; and
determining that inhibition of CTLA4 in a subject with melanoma will result in clinical benefit in the subject if the expression level of the melanoma-associated genes in the test sample is higher than the level of the gene in the reference sample.

23. The method of claim 1, wherein the test sample is obtained from the melanoma and the melanoma-associated gene comprises CD2, CD6, CXCL13, CD3D, CD3E, CD3G, LCK, T cell receptor alpha gene, T cell receptor beta gene, CD28, ICOS, EOMES, IL2RB, FASLG, SLAMF6, GNLY, GZMA, GZMB, GZMH, GZMK, PRF1, PTCRA, CD19, CD72, FCRL1/3, MS4A1, CTLA4, LAG3, FCRL1, FCRL3, CD5L, SIGLEC8, or FAIM3/TOSO; and
determining that inhibition of CTLA4 in a subject with melanoma will result in clinical benefit in the subject if the expression level of the melanoma-associated gene in the test sample is higher than the level of the melanoma-associated gene in the reference sample.

24. The method of claim 1, wherein the test sample is obtained from a melanoma tumor microenvironment, wherein the melanoma-associated gene comprises a T cell infiltration-associated gene, a receptor signaling gene, an activation gene, a cytotoxicity gene, a humoral immunity gene, or an immune inhibitory receptor gene; and
determining that inhibition of CTLA4 in a subject with melanoma will result in clinical benefit in the subject if the expression level of the melanoma-associated gene in the test sample is higher than the level of the gene in the reference sample.

25.-52. (canceled)

53. A composition for predicting no clinical benefit in response to CTLA4 therapy comprising a melanoma-associated gene, wherein the melanoma-associated gene comprises MAGEA2, MAGEA3, MAGEA6, MAGEA12, CSAG1, CSAG2, CSAG3, or CSAG4 synthesized complementary deoxyribonucleic acid (cDNA; or
a composition for predicting clinical benefit in response to CTLA4 therapy comprising miR-211 and a melanoma-associated gene selected from the group consisting of CD5L, IL12RB2, FAIM3, PTCRA, CD2, CD6, CXCL13, CD3D, CD3E, CD3G, LCK, T cell receptor alpha gene, T cell receptor beta gene, GNLY, GZMA, GZMB, GZMH, GZMK, PRF1, CD19, CD72, FCRL1/3, MS4A1, CTLA4, LAG3, FCRL1, FCRL3, SIGLEC8, and FAIM3/TOSO synthesized cDNA, or
a kit, comprising reagents for assaying a biological sample from a subject with cancer for:
(a) aberrant expression of at least one resistant cancer-associated gene or miRNA, or
(b) aberrant expression of at least one beneficial cancer-associated gene or miRNA.

54.-58. (canceled)

59. A method of treating cancer in a subject in need thereof, comprising: administering a therapeutically effective amount of one or more CTLA4 inhibitor agents to the subject, wherein the subject is identified as
(a) not having aberrant expression of at least one resistant cancer-associated gene or miRNA, or
(b) having aberrant expression of at least one beneficial cancer-associated gene or miRNA; or
a method of treating cancer in a subject in need thereof, comprising:
(a) analyzing a biological sample from the subject for:
(i) aberrant expression of at least one resistant cancer-associated gene or miRNA, wherein the aberrant expression of the at least one resistant cancer-associated gene or miRNA is not present in the biological sample, or
(ii) aberrant expression of at least one beneficial cancer-associated gene or miRNA, wherein the aberrant expression of the at least one beneficial cancer-associated gene or miRNA is present in the biological sample;
(b) identifying the subject as a candidate for receiving one or more CTLA4 inhibitor agents; and
(c) administering a therapeutically effective amount of the one or more CTLA4 inhibitor agents to the subject;
a method of treating cancer comprising administering an effective amount of a CTLA4 inhibitor and an effective amount of a HMGB1 receptor agonist; or
a method of treating cancer comprising administering an effective amount of a CTLA4 inhibitor and an effective amount of a Xg28-CGA antagonist; or
a method of treating cancer comprising administering an effective amount of a CTLA4 inhibitor and an effective amount of an agonist or inducer of autophagy; or
a method of treating cancer comprising administering an effective amount of a CTLA4 inhibitor and an effective amount of a agonist or inducer of miR-211, miR-185 and/or miR-513A2; or
a method of identifying a subject with cancer as a candidate for receiving one or more CTLA4 inhibitor agents, comprising:
(a) analyzing a biological sample from the subject for:
(i) aberrant expression of at least one resistant cancer-associated gene or miRNA, wherein the aberrant expression of the at least one resistant cancer-associated gene or miRNA is not present in the biological sample, or
(ii) aberrant expression of at least one beneficial cancer-associated gene or miRNA, wherein the aberrant expression of the at least one beneficial cancer-associated gene or miRNA is present in the biological sample; and
(b) identifying the subject as a candidate for receiving one or more ctla4 inhibitor agents.

60.-69. (canceled)

70. A method of determining whether administration of a CTLA4 inhibitor and a HMGB1 receptor agonist to a subject with melanoma will result in clinical benefit in the subject comprising:
obtaining a test sample from a subject having or at risk of developing melanoma;
determining the expression level of at least one melanoma-associated gene in the test sample;
comparing the expression level of the melanoma-associated gene in the test sample with the expression level of the melanoma-associated gene in a reference sample; and
determining whether administration of a CTLA4 inhibitor and a HMGB1 receptor agonist will inhibit melanoma in the subject if the expression level of the melanoma-associated gene in the test sample is differentially expressed as compared to the level of the melanoma-associated gene in the reference sample; or
a method of determining whether administration of a CTLA4 inhibitor and a Xq28-CGA antagonist to a subject with melanoma will result in clinical benefit in the subject comprising:
obtaining a test sample from a subject having or at risk of developing melanoma;
determining the expression level of at least one melanoma-associated gene in the test sample;
comparing the expression level of the melanoma-associated gene in the test sample with the expression level of the melanoma-associated gene in a reference sample; and
determining whether administration of a CTLA4 inhibitor and Xg28-CGA antagonist will inhibit melanoma in the subject if the expression level of the melanoma-associated gene in the test sample is differentially expressed as compared to the level of the melanoma-associated gene in the reference sample; or
a method of determining whether administration of a CTLA4 inhibitor and an autophagy agonist to a subject with melanoma will result in clinical benefit in the subject comprising:
obtaining a test sample from a subject having or at risk of developing melanoma;
determining the expression level of at least one melanoma-associated gene in the test sample;
comparing the expression level of the melanoma-associated gene in the test sample with the expression level of the melanoma-associated gene in a reference sample; and
determining whether administration of a CTLA4 inhibitor and an autophagy agonist will inhibit melanoma in the subject if the expression level of the melanoma-associated gene in the test sample is differentially expressed as compared to the level of the melanoma-associated gene in the reference sample;
a method of determining whether administration of a CTLA4 inhibitor and a miR-211, miR-185 and/or miR-513A2 agonist to a subject with melanoma will result in clinical benefit in the subject comprising:
obtaining a test sample from a subject having or at risk of developing melanoma;
determining the expression level of at least one melanoma-associated gene in the test sample;
comparing the expression level of the melanoma-associated gene in the test sample with the expression level of the melanoma-associated gene in a reference sample; and
determining whether administration of a CTLA4 inhibitor and an miR-211, miR-185 and/or miR-513A2 agonist will inhibit melanoma in the subject if the expression level of the melanoma-associated gene in the test sample is differentially expressed as compared to the level of the melanoma-associated gene in the reference sample.

71.-92. (canceled)
